(19) United States
(12) Patent Application Publication
(10) Pub. No.: US 2011/0257034 A1
(43) Pub. Date: Oct. 20, 2011

(54) METHODS FOR IDENTIFYING GENES WHICH PREDICT DISEASE OUTCOME FOR PATIENTS WITH COLON CANCER

(75) Inventors: Francis Barany, New York, NY (US); Owen Parker, Brooklyn, NY (US); Manny D. Bacolod, Roosevelt Island, NY (US); Sarah F. Giardina, New York, NY (US); Yu-wei Cheng, Chicago, IL (US); Daniel A. Notterman, Cranbury, NJ (US); Gunter S. Schemmann, Princeton, NJ (US); Philip B. Paty, New York, NY (US); Monib Zirvi, Monmouth Junction, NJ (US)

(73) Assignees: CORNELL UNIVERSITY, Ithaca, NY (US); THE TRUSTEES OF PRINCETON UNIVERSITY, Princeton, NJ (US); SLOAN-KETTERING INSTITUTE FOR CANCER RESEARCH, New York, NY (US); UNIVERSITY OF MEDICINE AND DENTISTRY OF NEW JERSEY, New Brunswick, NJ (US)

(21) Appl. No.: 13/123,689

(22) PCT Filed: Oct. 13, 2009
(86) PCT No.: PCT/US2009/005573

§ 371 Date: Jun. 29, 2011

Related U.S. Application Data
(60) Provisional application No. 61/104,574, filed on Oct. 10, 2008.

(51) Int. Cl. C40B 30/04; C40B 40/06; G01N 33/53; C40B 40/08; C12Q 1/68; G01N 33/566

(52) U.S. Cl. 506/9; 435/6.1; 435/6.18; 435/6.17; 436/501; 435/6.12; 435/6.11; 435/6.13; 506/17; 506/16

(57) ABSTRACT

Closures for containers and methods for using same are provided. In a general embodiment/the present disclosure provides a closure having a top portion (12), a bottom portion (14) and a side portion (16), an aperture (18) extending though the closure, a projection (20) extending from the closure and at least two rib members (36) on an interior of the projection. The projection may also include a cover (22). In another embodiment, a method for using a closure includes inserting a. spike member into a projection, piercing a membrane that hermetically seals a medical container, pushing rib members within the projection to center the spike member inserted into the projection, and tearing the membrane to create a vent hole in the membrane.

This application claims the benefit of U.S. Provisional Patent Application Ser. No. 61/104,574 filed Oct. 10, 2008, which is hereby incorporated by reference in its entirety.

This invention was made with government support under grant numbers P01-CA65930 and HHSN261200700388P, both awarded by the National Cancer Institute. The government has certain rights in this invention.

### FIELD OF THE INVENTION

The present invention is directed to methods of determining the prognosis of a subject having colon cancer. Collections of genes whose expression levels are informative of colon cancer prognosis are also disclosed.

### BACKGROUND OF THE INVENTION

Oncologists are often faced with difficult treatment decisions regarding the use of chemotherapy and adjuvant radiation therapy for various tumors. Patients and oncologists are increasingly looking for prognostic indicators to help them make these difficult decisions. Since these treatments have significant toxicity and inherent dangers, it is critical to have means to help determine prognosis and minimize adverse events as a result of over-treating patients who would have fared well without aggressive treatments.

With the advent of accurate and rapid means to analyze the RNA and DNA found in tumors, diagnostic tests that predict outcome are increasingly utilized in clinical settings to help guide treatment decisions for clinicians. In particular, patients who suffer from breast cancer have recently been able to have their tumors analyzed using molecular genetic techniques to help predict their disease outcome. This initial breast cancer prognostic test consisted of a mutation analysis of a small number of genes including, BRCA1, BRCA2, and BRCA3. Analysis of ErbB2 status has also been helpful in guiding patient treatment with targeted therapies such as Herceptin.

Although these initial analyses provided some useful information for a subset of breast cancer patients, it did not provide useful prognostic information for the vast majority of patients. Therefore, more recent attempts to provide prognostic information for breast cancer tumors have been based on gene expression patterns of multiple genes.

Several recent publications report the use of microarray gene expression analysis to characterize tumors such as breast cancers (Golub et al, “Molecular Classification of Cancer: Class Discovery and Class Prediction by Gene Expression Monitoring,” Science, 286(5439):531-537 (1999); Bhattarcharjee et al, “Classification of Human Lung Carcinomas by mRNA Expression Profiling Reveals Distinct Adenocarcinoma Subclasses,” Proceed. Natl. Acad. Sci. U.S.A., 98(24):13790-13795 (2001); Ramaswamy et al, “Multiclass Cancer Diagnosis Using Tumor Gene Expression Signatures,” Proceed. Natl. Acad. Sci. U.S.A., 98(26):15149-15154 (2001); Martin et al, “Linking Gene Expression Patterns to Therapeutic Groups in Breast Cancer,” Cancer Res., 60(8):2232-2238 (2000); West et al, “Predicting the Clinical Status of Human Breast Cancer by Using Gene Expression Profiles,” Proceed. Natl. Acad. Sci. U.S.A., 98(20):11462-11467 (2001)). These studies have shown gene expression patterns specific to breast cancer tumors that may have prognostic value. (Sorlie et al, “Gene Expression Patterns of Breast Carcinomas Distinguish Tumor Subclasses with Clinical Implications,” Proceed. Natl. Acad. Sci. U.S.A., 98(19):10869-10874 (2001); Yan et al, “Dissecting Complex Epigenetic Alterations in Breast Cancer Using CpG Island Microarrays,” Cancer Res., 61(23):8375-8380 (2001); Van De Vijver et al, “A Gene-Expression Signature as a Predictor of Survival in Breast Cancer,” N. Engl. J. Med., 347(25):1999-2009 (2002)). Using similar techniques, commercial products like Oncotype Dx (Genomic Health, Redwood City, Calif.) have been developed, making breast cancer prognosis widely available.

Similar testing for other cancers, such as colon cancer, are currently not available. This year, over 153,000 new cases of colorectal cancer (CRC) will be diagnosed, and 52,180 patients will die from this disease in the United States. There is an urgent need to improve colorectal cancer prognosis by developing accurate molecular techniques that will complement the clinico-pathology, as well as to identify individuals with early disease.

The present invention is directed to overcoming these and other deficiencies in the art.

### SUMMARY OF THE INVENTION

A first aspect of the present invention relates to a method for determining the prognosis of a subject having colon cancer that involves obtaining a biological sample from the subject and detecting expression levels of at least five genes selected from a group of 176 genes informative of colon cancer prognosis. The group of 176 genes informative of colon cancer prognosis includes the following genes: ACSL4, RQCD1, AA058828*, AIP, AKR1A1, AP3D1, ARL2BP, ARL4A, ARL6IP4, OGFOD2, ASNA1, ATP5B, C12orf52, C19orf36, C1GALT1, C1orf144, C5orf23, C6orf15, C7orf10, C8orf70, CALML4, CASP1, CCNA2, CCT2, CDC42BPA, AK023058*, CDR2L, CFB, CHST12, CLN5, CMPK1, CNOT7, CNPY2, COBL, COMMD4, COX5A, CXCL11, CYB561, CYB5B, DAZAP2, DDX23, DENND2A, DENND2D, DHX15, AL359599*, DND1, DOCK9, EGFR, ELP3, ERP29, ETV1, FAM82C, FDFT1, FKBP14, FLJ10357, FRYL, GALNS, GCHFR, GHITM, GLS, GPR177, GRB10, GREM2, GRHPR, GRP, GSR, GSTA1, H2AFZ, HOXB7, IFT88, IL15RA, ISG20, ITGAE, KIAA0746, SERINC2, KIF13B, KLC1, LAMP3, LANCL1, LAP3, LEPREL1, LL22NC03-5H6.5, LOC100131861, SAMM50, LRRC41, LRRC47, MAP4, MAPKAPK5, MCM5, MCRS1, METRN, METTL3, MFHAS1, MMP3, MOSPD1, MRPL46, MTUS1, MYRIP, N4BP2L2, NAB1, NAT1, NDUFC1, NISCH, NUMB, OGT, OSBPL3, PAM, PBK, PDGFA, PEBP1, PGDS, PIGR, PIGT, PRDM2, PRELP, PSMA5, PSMD9, PSPC1, PTHLH, R3HCC1, RP3-377H14.5, RPLP0, RPLP0-like, RPS27L, RTN2, RYK, SAV1, SCAMP1, SERPINA1, SF3B1, SFPQ, SGCD, SLC25A3, SLC39A8, SMG7, SMURF2, SORD, SOX4, SPATA5L1, SQRDL, SRP72, SSNA1, STK3, SYNGR1, TAPBPL, TEGT, TES, TLN1, TMCC1, TMEM106C, TMEM16A, TMEM33, TMEM87A, TNFRSF10B, TNFSF10, TNIK, TRIM36, U2AF2, UBE2L6, UCP2, UNC84A, UQCRFS1, UQCRH, USP12, USP3, VPS41, WARS, WDR1, WDR68, XPO7, YBX1, ZC3H7B, ZMYM2, ZMYM5, ZNF117, and ZNF430. This method further involves comparing the detected expression levels of the at least five genes from the biological sample with the expression levels of the corresponding at least five genes when associated with a good disease prognosis expression profile and when associated with a bad disease prognosis expression profile. Based on that comparison, the prognosis of the subject having colon cancer is determined.

Another aspect of the present invention relates to a method for determining the prognosis of a subject having colon cancer that involves obtaining a biological sample from the subject and detecting the expression level of at least five genes selected from a group of 101 genes informative of colon cancer prognosis. The group of 101 genes informative of colon cancer prognosis includes the following genes: NARS, WDR1, WARS, CCT4, ATP5B, SORD, UBE2L6, PSME2, AIP, RRM2, LRRC41, CCT2, TAF9, HDAC5, SVIL, CCNB2, DBN1, PBX2, RFC5, IDE, MAD2L1, PSMA4, NDUFC1, IVD, PPIH, NEO1, CXCL10, FXN, GABBR1, ARHGAP8, LOC553158, HOXA4, COMMD4, DFFB, KLF12, GLMN, CASP7, PIR, ATP5G3, ACTN1, DDOST, TAPBP, RGL2, CYB561, TUSC3, C3orf63, GRB10, NR2F1, WDR68, CXCL2, CNPY2, CASP1, INDO, PFKM, CXCL11, MCAM, MAP2K5, MRPS11, NOLC1, CD59, CAMSAP1L1, SHANK2, KLC1, EMP1, C1orf95, GMDS, RPLP0, RPLP0-like, PDLIM4, PAM, TM4SF1, BEX4, ADORA1, FAM48A, ITM2B, PREB, CMPK1, LAP3, FAM82C, AACS, RP5-1077B9.4, NUP37, RHBDF1, PBK, TIPIN, TMEM204, ALG6, NPR3, SCD5, FLJ13236, GPATCH4, GREM2, RPL22, KLHL3, C15orf44, USP3, TNS1, ZBTB20, RTN2, FLJ10357, and CALML4. This method further involves comparing the detected expression levels of the at least five genes from the biological sample with the expression levels of the corresponding at least five genes when associated with a good disease prognosis expression profile and when associated with a bad disease prognosis expression profile. Based on that comparison, the prognosis of the subject having colon cancer is determined.

The present invention is also directed to a method of identifying an agent that improves the prognosis of a subject having colon cancer. This method involves administering the agent to the subject having colon cancer and obtaining a first biological sample from the subject before said administering and a second biological sample from the subject after said administering. The method further involves detecting the expression levels of at least five genes selected from the group of 176 genes informative of colon cancer prognosis disclosed supra. Determining increases or decreases in the expression levels of the at least five genes in the second sample compared to the first sample identifies an agent that improves the prognosis of a subject having colon cancer.

Another aspect of the present invention is directed to a collection of 71 genes having expression levels informative for predicting a prognosis of a patient having colon cancer. The collection of 71 genes comprises the following genes: SLC25A3, DAZAP2, TEGT, ERP29, PSMA5, DDX23, LOC100131861, SAMM50, SFPQ, NISCH, CYB5B, TMEM106C, EGFR, MCRS1, SERPINA1, CCNA2, NDUFC1, COX5A, GCHFR, ITGAE, PRDM2, PDGFA, GSR, GRP, COMMD4, XPO7, YBX1, SRP72, UCP2, SLC39A8, NAB1, WDR68, CXCL11, RECQL, CASP1, PTHLH, UNC84A, MTUS1, KIAA0746, SERINC2, DOCK9, FRYL, MAPKAPK5, LRRC47, RQCD1, TNIK, RPLP0, RPLP0-like, CLN5, NAT1, CDC42BPA, GSTA1, ZMYM5, RYK, PIGT, CMPK1, SQRDL, FAM82C, CNOT7, LL22NC03-5H6.5, PSPC1, TAPBPL, METRN, PBK, MRPL46, FKBP14, C1GALT1, GREM2, GPR177, DND1, and PRELP.

Another aspect of the present invention is related to a collection of 101 genes having expression levels informative for predicting a prognosis of a patient having colon cancer. The collection of 101 genes comprises the following genes: AACS, ACTN1, ADORA1, AIP, ALG6, ARHGAP8, L00553158, ATP5B, ATP5G3, BEX4, C15orf44, C1orf95, C3orf63, CALML4, CAMSAP1L1, CASP1, CASP7, CCNB2, CCT2, CCT4, CD59, CMPK1, CNPY2, COMMD4, CXCL10, CXCL11, CXCL2, CYB561, DBN1, DDOST, DFFB, EMP1, FAM48A, FAM82C, FLJ10357, FLJ13236, FXN, GABBR1, GLMN, GMDS, GPATCH4, GRB10, GREM2, HDAC5, HOXA4, IDE, INDO, ITM2B, IVD, KLC1, KLF12, KLHL3, LAP3, LRRC41, MAD2L1, MAP2K5, MCAM, MRPS11, NARS, NDUFC1, NEO1, NOLC1, NPR3, NR2F1, NUP37, PAM, PBK, PBX2, PDLIM4, PFKM, PIR, PPIH, PREB, PSMA4, PSME2, RFC5, RGL2, RHBDF1, RP5-1077B9.4, RPL22, RPLP0, RPLP0-like, RRM2, RTN2, SCD5, SHANK2, SORD, SVIL, TAF9, TAPBP, TIPIN, TM4SF1, TMEM204, TNS1, TUSC3, UBE2L6, USP3, WARS, WDR1, WDR68, and ZBTB20.

The current standard of care for colorectal cancer provides the average treatment for the average tumor, with less than average results. Current cancer care over-treats many patients to help an unknown few, with toxic, relatively ineffective, expensive therapeutics. There is an urgent need to develop a means to predict which patients will respond to standard therapies, which patients do not require therapy in addition to surgery, and which patients are likely not to respond to current therapeutics. For every 100 stage 11 and III colon cancer patients on adjuvant therapy, only about 12 of them will respond favorably, about 50 would survive without therapy, and about 38 will experience a recurrence even when given the current treatments. The current invention seeks to help individuals on both sides of this equation by stratifying the risk of a poor outcome. Thus, individuals with low risk tumors, in consultation with their physicians, may opt to avoid unnecessary and debilitating therapy. On the other hand, individuals with high risk tumors may seek to enroll in clinical trials testing the newest therapies to increase their chance of a better outcome.

### BRIEF DESCRIPTION OF THE DRAWINGS

FIG. 1 is a flow chart outlining methods for determining the prognosis of a subject having colon cancer in accordance with the present invention. Tumor tissue RNA is harvested and converted to cDNA using reverse transcription. The cDNA is then hybridized to an expression array to determine gene expression levels. Tumor tissue DNA is analyzed for microsatellite instability, gene promoter methylation, and mutational status. Data from one or more analyses is used to determine a subject's prognosis and develop a personalized treatment plan.

FIG. 2 is a flow chart depicting the steps used to identify the 176 and 71 gene predictor sets of the present invention that are useful for predicting disease outcome in subjects having colon cancer.

FIGS. 3A-3B illustrate how a patient's outcome is determined using the expression levels of the 71, 101, or 176 gene predictor sets of the present invention. FIG. 3A outlines the steps taken to determine, in a sample taken from a patient having colon cancer, the prognosis of that patient based on the expression levels of the genes in the 71-, 101-, or 176 genes sets and FIG. 3B applies the steps outlined in FIG. 3A to three hypothetical samples where the expression levels of six genes were determined.

FIG. 4 is a scatterplot graphing the predicted outcome for 166 stage I-IV primary colon cancer tumor samples based on gene expression levels of the 71-genes in the 71-gene predictor set. The x-axis depicts the percentage of genes for a given tumor sample that had expression values associated with a bad disease outcome. The y-axis depicts the percentage of genes for a given tumor sample that had expression values associated with a good disease outcome. Tumor samples from patients who died of disease (DOD) (n=56) are represented by (▪), while tumor sample from all other patients who survived or died of other causes (non-DOD) (n=110) are represented by (♦). Samples which binned to Group 1 had good prognosis with only 6% being categorized as DOD. Samples which binned to Group 4 had poor prognosis with 70% being categorized as DOD. Groups 2 and 3 had intermediate prognosis levels.

FIGS. 5A-5E are scatterplots graphing the predicted outcomes for the 166 stage I-IV primary colon cancer tumor samples based on gene expression levels of the 71-genes in the 71-gene predictor set stratified into high, intermediate, and low risk groups with the stage and recurrence status of the tumor identified. FIG. 5A is the same plot as shown in FIG. 4 with further stratification. The percentage of DOD patients increases steadily in each subgroup from Group 1 (0%) to Group 2A+2B (14%) to Group 3A+3B (42%) to Group 4 (69%) to Group 5+6 (83%). In FIG. 5B, stage I tumors are identified. Most stage I tumors binned to low risk groups 1 and 2A. One recurrence was identified in this group (i.e. stage I tumor) and is noted on the graph (“R68”). The recurrence was detected after 68 months, and, therefore, it is unclear if it is a recurrence or a new tumor. In FIG. 5C, stage II tumors are identified. Stage II tumor samples are spread evenly through the risk groups. Three recurrences were identified and binned to group 3B and the border of group 2A/2B. In FIG. 5D, the stage III tumors are identified. Surprisingly, a number of stage III tumor samples binned to Group 1 showing that analysis of gene expression of the 71-gene predictor set is not simply recapitulating tumor stage. Recurrences in the stage III population of samples were identified in all risk groups. FIG. 5E shows the stage IV tumor samples. These samples binned as predicted, mostly to groups 4-6 (i.e. high risk).

FIG. 6 is a scatterplot graphing the predicted outcome for the 166 stage I-IV primary colon cancer tumor samples based on gene expression levels of the 1389-genes in the 1389-gene predictor set. The x-axis depicts the percentage of genes for a given tumor sample that had expression values associated with a bad disease outcome. The y-axis depicts the percentage of genes for a given tumor sample that had expression values associated with a good disease outcome. Tumor samples from DOD patients are represented by (▪), while tumor sample from non-DOD patients (n=110) are represented by (♦). The stratification of survival outcome did not improve significantly between the 71 gene set and the 1389 gene set.

FIG. 7 is a scatterplot graphing the predicted outcome for the 166 stage I-IV primary colon cancer tumor samples based on gene expression levels of the 101 genes in the 101-gene predictor set ranked by the odds ratio analysis. The x-axis depicts the percentage of genes for a given tumor sample that had expression values associated with a bad disease outcome. The y-axis depicts the percentage of genes for a given tumor sample that had expression values associated with a good disease outcome. Tumor samples from DOD patients are represented by (▪), while tumor sample from non-DOD patients (n=110) are represented by (♦). The low risk category can be segregated from the intermediate and high risk categories by the lines indicated on the graph.

FIG. 8 is a scatterplot graphing the predicted outcome for the 166 stage I-IV primary colon cancer tumor samples based on gene expression levels of the 101 genes in the 101-gene predictor set ranked by difference scores. The x-axis depicts the percentage of genes for a given tumor sample that had expression values associated with a bad disease outcome. The y-axis depicts the percentage of genes for a given tumor sample that had expression values associated with a good disease outcome. Tumor samples from DOD patients are represented by (▪), while tumor sample from non-DOD patients (n=110) are represented by (♦). The low risk category had 2% of patients who were in the DOD category. The high risk group by contrast had 87% of patients in the DOD category. The intermediate risk had 56% of patients in the DOD category.

FIG. 9 is a scatterplot graphing the predicted outcome for the 166 stage I-IV primary colon cancer tumor samples based on gene expression levels of the 71 genes in the 71-gene predictor set as shown in FIG. 4 with LRAT methylation status of various samples identified (see arrows). Several DOD samples that had binned to group 1 based on gene expression levels had low to no LRAT methylation, which predicts poor prognosis. Removing these samples from group 1 based on LRAT methylation status improved the performance of the prognosis prediction in the low risk category. The low risk category in this analysis only had 3% of patients in the DOD category.

FIG. 10 is a scatterplot graphing the predicted outcome for the 166 stage I-IV primary colon cancer tumor samples based on gene expression levels of the 71 genes in the 71-gene predictor set stratified into high, intermediate, and low risk groups. The LRAT methylation status of various samples is also identified. As in FIG. 9, when LRAT methylation status was included in the analysis, the low risk groups had excellent prediction of good outcome. Group 1 does not contain patients with DOD status while Group 2A+2B only has 6% of patients with DOD status.

FIG. 11 is a scatterplot graphing the predicted outcome for the 166 stage I-IV primary colon cancer tumor samples based on gene expression levels of the 101 genes in the 101-gene predictor set ranked by difference score. The LRAT methylation status of various samples is also identified. The x-axis depicts the percentage of genes for a given tumor sample that had expression values associated with a bad disease outcome. The y-axis depicts the percentage of genes for a given tumor sample that had expression values associated with a good disease outcome. Tumor samples from DOD patients are represented by (▪), while tumor sample from non-DOD patients (n=110) are represented by (♦). Inclusion of LRAT methylation status was useful to reclassify some patient outcomes in this example as well.

FIG. 12 is the overall view of gene expression dysregulation in regions of chromosomal aberrations. Shown are the percentages of samples with copy number gains (top chart), copy number losses (middle chart), and copy neutral-LOH events (bottom chart) in every autosomal chromosome. Each circle represents a gene located in the region of aberration, and whose colon cancer expression is at least 3 standard deviation units above (red) or below (green) the baseline (normal mucosa samples) for at least 10% of the colon cancer samples. As evident in the population of the colored circles, there are more upregulated genes in regions of gains, and more downregulated genes in regions of losses.

FIG. 13 is a numerical representation of FIG. 12. It shows the percentages of genes that have: a) gained copy number and increased expression level (red bar), b) lost copy number and decreased expression level (green bar), c) gained copy number and decreased expression level (gray bar, pointing down), and d) lost copy number and increased expression level (gray bar, pointing up). The percentages are calculated based on the number of unique genes in every chromosome arm. As shown in this chart, chromosome arms 7p, 7q, 8q, 13q, 20p, and 20q have high proportion of upregulated genes. On the other hand, 1p, 4q, 8p, 14q, 15q, 17p, 18p, and 18q have high proportion of downregulated genes.

FIG. 14 shows genes that have dysregulated expression on chromosome 8. In general, genes which are upregulated correlate with regions of copy number gain and genes which are downregulated correlate with regions of copy number loss. The 8q arm, containing numerous regions of gain, includes the genes NCO6AIP (or TGS1), CHD7, DPY19L4, LAPTM4B, PABPC3, SLC25A32, and EIF2C2 which all have elevated expression. The 8p arm, containing numerous regions of loss, includes the highly downregulated genes MTUS1, ADAMEC1, EPHX2, TMEM64, and PPP2CB.

FIG. 15 is a graph summarizing the Kaplan-Meier (KM) survival curve analyses done for the most highly dysregulated genes in the widely recognized aneuploidy regions in colorectal cancer. Shown are the percentages (fractions indicated on each bar) of the most highly dysregulated genes in chromosomes 7, 8p, 13q, 17p, 18, 20p, and 20q where expression levels are concordant (red for the gained and green for the lost arms) or discordant (gray bars) with prognosis.

FIGS. 16A-16J are Kaplan-Meier survival curves for 10 of the 13 most dysregulated genes on chromosomal arm 8p. Included in each graph is the Affymetrix probe identifier, gene name, and chromosome location. In each case, lower expression (shown in red) correlated with worse outcome, consistent with chromosomal loss contributing to bad prognosis. Higher expression is shown in green.

FIGS. 17A-17B show the distribution of the 71 gene set among different autosomal chromosomal arms. FIG. 17A shows chromosomes 1-7, while FIG. 17B shows chromosomes 8-22 and X. In general, the expression pattern of the 71 gene set followed the pattern of chromosomal copy number dysregulation observed in the colon tumors analyzed. The number of dysregulated genes in each chromosomal arm predicting outcome based on expression is indicated. Copy loss (green), gain (red), and copy neutral LOH (yellow) are demonstrated across the chromosomal arms.

FIGS. 18A-18B show the distribution of the 176 gene set among different chromosomal arms. FIG. 18A shows chromosomes 1-7, while FIG. 18B shows chromosomes 8-22 and X. In general, the expression pattern of the 176 gene set followed the pattern of chromosomal copy number dysregulation observed in the colon tumors analyzed. The number of dysregulated genes in each chromosomal arm predicting outcome based on expression is indicated. Copy loss (green), gain (red), and copy neutral LOH (yellow) are demonstrated across the chromosomal arms.

FIG. 19 is the Kaplan-Meier survival curve for Caspase 1, one of the genes of the 71 gene predictor set. The red line indicates survival for patients having tumors where the expression of Caspase 1 is in the top third of average tumor expression. The green line indicates survival for patients having tumors where the expression of Caspase 1 is in the middle third of average tumor expression. The blue line indicates survival for patients having tumors where the expression of Caspase 1 is in the bottom third of average tumor expression. When the expression level of Caspase 1 is in the top third of average tumor expression a favorable prognosis is predicted and when the expression level is in the bottom third of average tumor expression an unfavorable prognosis is predicted.

FIG. 20 is a Kaplan-Meier survival curve for the TMEM106C gene showing a skewed distribution. When TMEM106C gene expression is in the lower third, relative to the average tumor expression level, a bad prognosis is predicted as indicated by the low percentage of survival in the KM curve (blue line). The percent survival was the same for tumors having average (middle third, green line) and above average (top third, red line) TMEM106C expression. Based on this analysis, this transmembrane protein is believed to have an important role in tumor progression.

FIG. 21 is a schematic diagram of enzymes and protein factors involved in retinol metabolism.

FIGS. 22A-22B show the LRAT methylation status for 69 samples that were classified as having microsatellite instability by either the three marker criteria (FIG. 22A) or the NCI criteria (FIG. 22B).

FIG. 23 shows the disease specific Kaplan-Meier survival analysis for LRAT methylation status. Only CRC tumor samples of all four clinical stages which were MSS (Microsatellite stable) were included in the survival analysis. The log-rank test shows a chi-square=4.73 and p-value=0.0296.

FIG. 24 shows the disease specific Kaplan-Meier survival analysis for LRAT methylation status. CRC tumor samples of all four clinical stages were included in the survival analysis. The log-rank test shows a chi-square=4.73 and p-value=0.0296.

FIG. 25 shows the disease specific Kaplan-Meier survival analysis for LRAT methylation status and retinoic acid receptor-β(RAR-β) methylation status. CRC tumor samples of all four clinical stages were included in the survival analysis.

FIG. 26 is a scatterplot graphing the predicted outcome for 22 additional primary colon tumor samples from patients that were not included in the original analysis of the 166 tumor set. There was excellent correlation between the predicted outcome and survival for samples in Group 1 as illustrated by the lack of samples from patients who DOD binning to Group 1.

FIG. 27 is a scatterplot graphing the predicted outcome for 36 liver metastases specimens generated using the 71 gene predictor set of the present invention. This analysis was performed to validate the 71 gene set on more advanced tumor samples. As shown, the vast majority of these specimens which included many that had DOD status binned to Group 4.

FIG. 28 is a scatterplot graphing the predicted outcome for 19 lung metastases specimens generated using the 71 gene predictor set of the present invention. This analysis was done to validate the 71 gene set on more advanced tumor samples. As shown, the vast majority of these specimens which included many that had DOD status binned to Group 4.

FIG. 29 is a scatterplot graphing the predicted outcome for 46 large primary adenoma specimens generated using the 71 gene predictor set of the present invention. The adenoma expression profiles in general predicted a low risk as most samples binned to Group 1. The few samples that did have DOD status also have either a synchronous primary tumor or synchronous metastases. It is important to note that the gene expression profiles of the primary colon tumors or metastatic tumors, in general predicted a poor outcome for survival as seen in the previous figures.

FIG. 30 is a scatterplot graphing the predicted outcome for 48 mucosa samples taken adjacent to a primary tumor sample. There are some mucosal samples, in which the results of this analysis may predict a poor outcome as a result of a field effect for genes that are dysregulated in the mucosa prior to the onset of a primary colon carcinoma.

FIG. 31 is a scatterplot graphing the predicted outcome for both normal mucosa and matched adjacent primary colon tumors. In this figure each matched pair is labeled with the same letter. The normal mucosa is marked in green and the tumor samples are marked in red. In general, the normal mucosa samples predict a better outcome in each case than the matched tumors. Also some tumors show greater changes in their expression profiles than others. This distribution may be a result of a combination of genes predisposing to the development of tumors, as well as, genes that contribute to poor outcome once a primary tumor has become aggressive and metastatic.

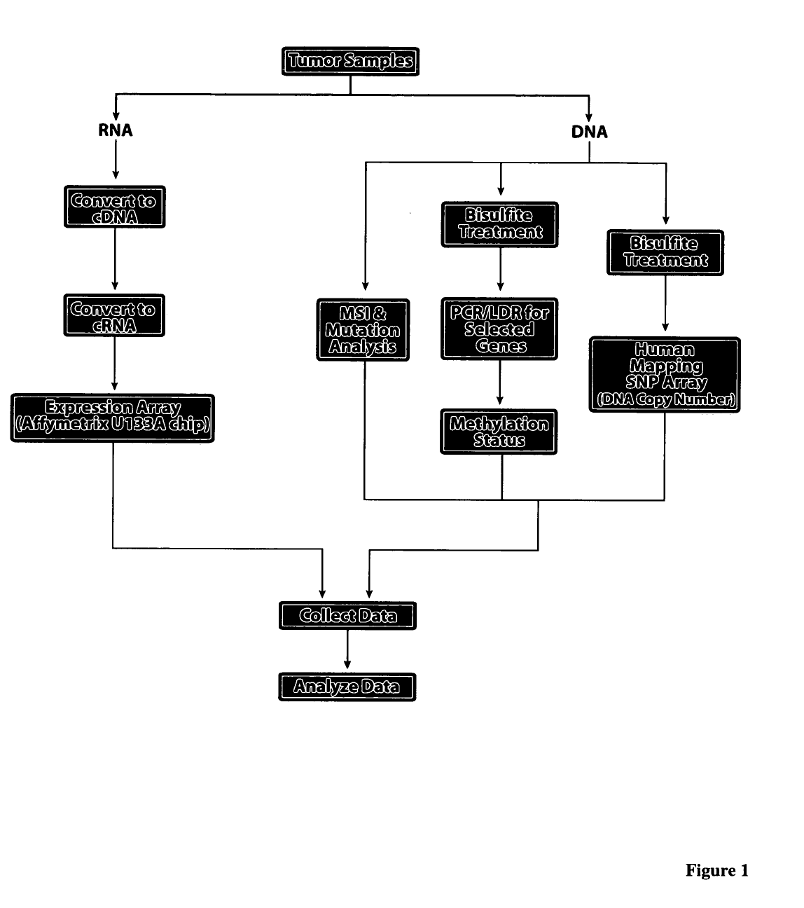

### DETAILED DESCRIPTION OF THE INVENTION

The present invention relates generally to methods of determining the prognosis of a subject having colon cancer. In a first aspect of the present invention, the method for determining the prognosis of a subject having colon cancer involves obtaining a biological sample from the subject and detecting expression levels of at least five genes selected from the group of 176 genes informative of colon cancer prognosis. The group of 176 genes informative of colon cancer prognosis includes the following genes: ACSL4, RQCD1, AA058828*, AIP, AKR1A1, AP3D1, ARL2BP, ARL4A, ARL6IP4, OGFOD2, ASNA1, ATP5B, C12orf52, C19orf36, C1GALT1, C1orf144, C5orf23, C6orf15, C7orf10, C8orf70, CALML4, CASP1, CCNA2, CCT2, CDC42BPA, AK023058*, CDR2L, CFB, CHST12, CLN5, CMPK1, CNOT7, CNPY2, COBL, COMMD4, COX5A, CXCL11, CYB561, CYB5B, DAZAP2, DDX23, DENND2A, DENND2D, DHX15, AL359599*, DND1, DOCK9, EGFR, ELP3, ERP29, ETV1, FAM82C, FDFT1, FKBP14, FLJ10357, FRYL, GALNS, GCHFR, GHITM, GLS, GPR177, GRB10, GREM2, GRHPR, GRP, GSR, GSTA1, H2AFZ, HOXB7, IFT88, IL15RA, ISG20, ITGAE, KIAA0746, SERINC2, KIF13B, KLC1, LAMP3, LANCL1, LAP3, LEPREL1, LL22NC03-5H6.5, LOC100131861, SAMM50, LRRC41, LRRC47, MAP4, MAPKAPK5, MCM5, MCRS1, METRN, METTL3, MFHAS1, MMP3, MOSPD1, MRPL46, MTUS1, MYRIP, N4BP2L2, NAB1, NAT1, NDUFC1, NISCH, NUMB, OGT, OSBPL3, PAM, PBK, PDGFA, PEBP1, PGDS, PIGR, PIGT, PRDM2, PRELP, PSMA5, PSMD9, PSPC1, PTHLH, R3HCC1, RP3-377H14.5, RPLP0, RPLP0-like, RPS27L, RTN2, RYK, SAV1, SCAMP1, SERPINA1, SF3B1, SFPQ, SGCD, SLC25A3, SLC39A8, SMG7, SMURF2, SORD, SOX4, SPATA5L1, SQRDL, SRP72, SSNA1, STK3, SYNGR1, TAPBPL, TEGT, TES, TLN1, TMCC1, TMEM106C, TMEM16A, TMEM33, TMEM87A, TNFRSF10B, TNFSF10, TNIK, TRIM36, U2AF2, UBE2L6, UCP2, UNC84A, UQCRFS1, UQCRH, USP12, USP3, VPS41, WARS, WDR1, WDR68, XPO7, YBX1, ZC3H7B, ZMYM2, ZMYM5, ZNF117, and ZNF430. This method further involves comparing the detected expression levels of the at least five genes from the biological sample with the expression levels of the corresponding at least five genes when associated with a good disease prognosis expression profile and when associated with a bad disease prognosis expression profile. Based on that comparison, the prognosis of the subject having colon cancer is determined.

In a preferred embodiment of this aspect of the present invention, the at least five genes are selected from a group of 71 genes informative of colon cancer prognosis. This group of 71 genes is a subset of the 176 genes informative of colon cancer prognosis and includes the following genes, SLC25A3, DAZAP2, TEGT, ERP29, PSMA5, DDX23, LOC100131861, SAMM50, SFPQ, NISCH, CYB5B, TMEM106C, EGFR, MCRS1, SERPINA1, CCNA2, NDUFC1, COX5A, GCHFR, ITGAE, PRDM2, PDGFA, GSR, GRP, COMMD4, XPO7, YBX1, SRP72, UCP2, SLC39A8, NAB1, WDR68, CXCL11, RECQL, CASP1, PTHLH, UNC84A, MTUS1, KIAA0746, SERINC2, DOCKS, FRYL, MAPKAPK5, LRRC47, RQCD1, TNIK, RPLP0, RPLP0-like, CLN5, NAT1, CDC42BPA, GSTA1, ZMYM5, RYK, PIGT, CMPK1, SQRDL, FAM82C, CNOT7, LL22NC03-5H6.5, PSPC1, TAPBPL, METRN, PBK, MRPL46, FKBP14, C1GALT1, GREM2, GPR177, DND1, and PRELP.

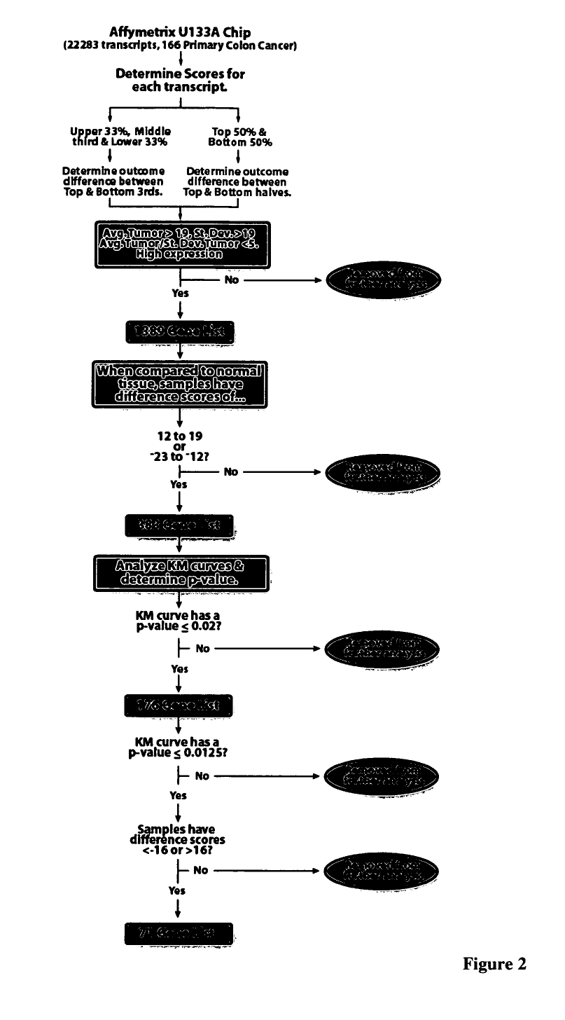

As described in greater detail in the Examples below, the 176- and 71-genes, whose expression levels are informative for predicting colon cancer outcome were derived from a larger pool of 383 genes. Kaplan-Meier (KM) survival curves were generated for the 383-genes and genes having p-values of >0.02 were removed from further analysis. The remaining group of 176 genes was further narrowed to 71 genes by removing genes having p-values associated with the KM curves of >0.0125 (See FIG. 2). Although a preferred embodiment of the invention, involves determining the prognosis of a subject having colon cancer by detecting the expression levels of at least five genes selected from the group of 176 or 71 genes, the expression levels of any five of the 383 genes also provides valuable prognostic information. The 383 genes, including the 176- and 71-genes whose expression levels are informative for the prediction of colon cancer are listed in Table 1, by gene symbol, alternative gene name(s), and Genbank Accession Number. The nucleotide sequences of the Affymetrix probes used to identify and quantify gene expression levels are also provided.

TABLE 1
383 Genes Informative of Colon Cancer Prognosis

| | Gene | | | Accession # | |
|---|---|---|---|---|---|
| Probe Set | Symbol | | | (Entrez Gene | |
| ID | (*identifier) | AKA | Gene Title | ID) | Probe Sequence(s) |
| 222033_s_at | AA058828* | | | | TAACAAAGAACGAGCACTTCCTTTC (SEQ ID NO: 1) |
| | | | | | GCACTTCCTTTCAGAGTTTCTGAGA (SEQ ID NO: 2) |
| | | | | | GGAACAGTCTGGGTGGAATGGGGCT (SEQ ID NO: 3) |
| | | | | | GGGGCTGAAACCATGTGCAAGTCTG (SEQ ID NO: 4) |
| | | | | | GAGATGTTAATTTTAGGGACCCGTG (SEQ ID NO: 5) |
| | | | | | TGTTTCCTAGCCCACAAGAATGCAA (SEQ ID NO: 6) |
| | | | | | AACAGATACTCGCTAGCCTCATTTA (SEQ ID NO: 7) |
| | | | | | AAAGGAGGAGTGCATCTTTGGCCGA (SEQ ID NO: 8) |
| | | | | | GCATCTTTGGCCGACAGTGGTGTAA (SEQ ID NO: 9) |
| | | | | | GGTTTGGTCCTATATTTCTAGTCAT (SEQ ID NO: 10) |
| | | | | | GTATTTTGTATACCATCTTCATATA (SEQ ID NO: 11) |
| 63825_at | ABHD2 | HS1-2, LABH2, | abhydrolase domain | 11057 | TGAAGTGCAACTGAAAGCTGCTAGT (SEQ ID NO: 12) |
| | | MGC111112, | containing 2 | | ACTGAAAGCTGCTAGTGATGATCTG (SEQ ID NO: 13) |
| | | MGC26249, | | | CTGAAAGCTGCTAGTGATGATCTGG (SEQ ID NO: 14) |
| | | PHPS1-2 | | | GTAATATACAATTTGTCCAGTAGCC (SEQ ID NO: 15) |
| | | | | | AATATACAATTTGTCCAGTAGCCAG (SEQ ID NO: 16) |
| | | | | | AATTTGTCCAGTAGCCAGTTTGTTT (SEQ ID NO: 17) |
| | | | | | AATGGTCACCGCAGGCCATACTACC (SEQ ID NO: 18) |
| | | | | | TCACCGCAGGCCATACTACCAATGA (SEQ ID NO: 19) |
| | | | | | CCATACTACCAATGAAATGGTAGGT (SEQ ID NO: 20) |
| | | | | | GAAATAGCACTCTGCATGCTTTGCT (SEQ ID NO: 21) |
| | | | | | AGCACTCTGCATGCTTTGCTCTACA (SEQ ID NO: 22) |
| | | | | | GCACTCTGCATGCTTTGCTCTACAA (SEQ ID NO: 23) |
| | | | | | CACTCTGCATGCTTTGCTCTACAAG (SEQ ID NO: 24) |
| | | | | | ACTCTGCATGCTTTGCTCTACAAGA (SEQ ID NO: 25) |
| | | | | | CTCTGCATGCTTTGCTCTACAAGAT (SEQ ID NO: 26) |
| | | | | | TCTGCATGCTTTGCTCTACAAGATG (SEQ ID NO: 27) |
| 202422_s_at | ACSL4 | ACS4, FACL4, | acyl-CoA synthetase | 2182 | ATTTTGATCTCACATTTTTATACCT (SEQ ID NO: 28) |
| | | LACS4, MRX63, | long-chain family | | AAATTGCTCACAGTTATTACATACA (SEQ ID NO: 29) |
| | | MRX68 | member 4 | | ATTACATACACATCAGCCATCAACT (SEQ ID NO: 30) |
| | | | | | ACACATCAGCCATCAACTAAAGTTG (SEQ ID NO: 31) |
| | | | | | ACTACAATATGTACATTTCTAAGTC (SEQ ID NO: 32) |
| | | | | | CTTTTGCTTTAATTCCATGAATGTT (SEQ ID NO: 33) |
| | | | | | ATGTTTCTTTGATCATGAATAACTG (SEQ ID NO: 34) |
| | | | | | GTAAGTCTTGCCTATATTTTCCTGA (SEQ ID NO: 35) |
| | | | | | TATTTTCCTGATAGCATATGACAAA (SEQ ID NO: 36) |
| | | | | | TATTCATATTTGTACTTTCTGTTAA (SEQ ID NO: 37) |
| | | | | | GTTTTTAAGTTTGCACTCAAATCTT (SEQ ID NO: 38) |
| 201782_s_at | AIP | ARA9, FKBP16, | aryl hydrocarbon | 9049 | GTGCCACTTATCCACCAGGAGGGCA (SEQ ID NO: 39) |
| | | FKBP37, | receptor interacting | | AGGAGGCTGCTGCCAAGTACTACGA (SEQ ID NO: 40) |
| | | SMTPHN, XAP2 | protein | | CAAGTACTACGATGCCATTGCCTGC (SEQ ID NO: 41) |
| | | | | | CATTGCCTGCCTCAAGAACCTGCAG (SEQ ID NO: 42) |
| | | | | | CCTGGGTCCCCTGAATGGATCCAGC (SEQ ID NO: 43) |
| | | | | | GAGGAGTACTACGAGGTGCTGGACC (SEQ ID NO: 44) |
| | | | | | GTCAAGGCCTACTTCAAGCGGGGCA (SEQ ID NO: 45) |
| | | | | | CGGCCGTGTGGAATGCCCAGGAGGC (SEQ ID NO: 46) |
| | | | | | GCCTGTGGTGAGCCGAGAGCTGCGG (SEQ ID NO: 47) |
| | | | | | CCTGGAGGCACGGATCCGGCAGAAG (SEQ ID NO: 48) |
| | | | | | GGGATCTTCTCCCATTGACAGGAGC [(SEQ ID NO: 49) |
| 222159_at | AK023058 | | CDNA FLJ12996 fis, | | TAGGAAATCCAGCAATCCTGGCCCC (SEQ ID NO: 50) |
| | | | clone | | CAGAAGTTCCCCCTAAGAACCACTG (SEQ ID NO: 51) |
| | | | NT2RP3000235 | | AAGGGGAGCCTCCTATGAGAATTCC (SEQ ID NO: 52) |
| | | | | | ATGAGAATTCCCCTAGTGATTCCCT (SEQ ID NO: 53) |
| | | | | | TGATTCCCTGTGGAGGTGGTTCCCA (SEQ ID NO: 54) |
| | | | | | AGGCTCATCTGCCCGAGGAGAGAAG (SEQ ID NO: 55) |
| | | | | | GAGGAGATTGGCACAGTGGGGTGGC (SEQ ID NO: 56) |
| | | | | | ATCAGCATCTAGTCATGGAGCTCCA (SEQ ID NO: 57) |
| | | | | | ATAAATCAATCTGTCTTTCTCACTG (SEQ ID NO: 58) |
| | | | | | AAGCAACATGGCATTCACGCAGGGC (SEQ ID NO: 59) |
| | | | | | GGCATTCACGCAGGGCCTGGTAATG (SEQ ID NO: 60) |
| 201900_s_at | AKR1A1 | ALDR1, ALR, | aldo-keto reductase | 10327 | GCCTGTCCAACTTCAACAGTCGGCA (SEQ ID NO: 61) |
| | | ARM, DD3, | family 1, member A1 | | TGATGACATACTCAGTGTGGCCTCC (SEQ ID NO: 62) |
| | | MGC12529, | (aldehyde reductase) | | GTAACTGCTTATAGCCCTTTGGGCT (SEQ ID NO: 63) |
| | | MGC1380 | | | CGTGCATGGCGTGATCCTGATGAGC (SEQ ID NO: 64) |
| | | | | | GAAAAGTATGGCCGATCTCCAGCTC (SEQ ID NO: 65) |
| | | | | | AGCGGAAAGTGATCTGCATCCCCAA (SEQ ID NO: 66) |
| | | | | | CTCCTTCTCGAATCCTTCAGAACAT (SEQ ID NO: 67) |
| | | | | | TTGACTTCACCTTTAGCCCAGAAGA (SEQ ID NO: 68) |
| | | | | | ATATTGTGCCTATGCTTACGGTGGA (SEQ ID NO: 69) |
| | | | | | TGTACCCCTTTAATGACCCGTACTG (SEQ ID NO: 70) |
| | | | | | CAGCTCTGCAGCTAATGAGGTCCTG (SEQ ID NO: 71) |
| 215828_at | AL359599* | | MRNA; cDNA | | CAATTAAACTTTTCTGCTTGCTCTC (SEQ ID NO: 72) |
| | | | DKFZp547C126 | | AATGATCTGGTCTGCTGGAAGGTGG (SEQ ID NO: 73) |
| | | | (from clone | | TGGGCAGTGGGTTGAGCGTGCAGCC (SEQ ID NO: 74) |
| | | | DKFZp547C126) | | GACTTGCACCTTTTCAGCAGTTATC (SEQ ID NO: 75) |
| | | | | | CAGCAGTTATCTAGACCTGGCCGCA (SEQ ID NO: 76) |
| | | | | | CCGCAGTGGCCTGAGTTGCAGTGAT (SEQ ID NO: 77) |
| | | | | | GATTCGTATTCACTCATTCTTTGAT (SEQ ID NO: 78) |
| | | | | | AGGGTTTTGTGCGTGTTGTTTCAGC (SEQ ID NO: 79) |
| | | | | | TATGACACGTGGCACCAGCTGTACA (SEQ ID NO: 80) |
| | | | | | TGCCCTTCTGGTTAGTGTTGTTCTT (SEQ ID NO: 81) |
| | | | | | GAAGCATTGATACTGTGGCCACTCA (SEQ ID NO: 82) |
| 222108_at | AMIGO2 | ALI1, DEGA | adhesion molecule | 347902 | TATTCAGCCAAAATTACCGTTTTAG (SEQ ID NO: 83) |
| | | | with Ig-like domain 2 | | AATGCCACATCCTATATGGTGTTAT (SEQ ID NO: 84) |
| | | | | | GAAAGTACATTTGTTTGCCTGTCTT (SEQ ID NO: 85) |
| | | | | | TTCATTTTGTACATTCTTCCCATTC (SEQ ID NO: 86) |
| | | | | | AAGTCATCATAATTTGTTGCCATAA (SEQ ID NO: 87) |
| | | | | | GAGTAACTTCTTAAATCCCTGTTCT (SEQ ID NO: 88) |
| | | | | | AAATCCCTGTTCTAGCAAACTAATA (SEQ ID NO: 89) |
| | | | | | ATATTGGTTCATGTGCTTGTGTATA (SEQ ID NO: 90) |
| | | | | | AATAGACCCTACTGTACTGTGCTTT (SEQ ID NO: 91) |
| | | | | | ACTGTACTGTGCTTTGGACATTTGA (SEQ ID NO: 92) |
| | | | | | TCACTGGACCGACGTTTTAACAACG (SEQ ID NO: 93) |
| 220144_s_at | ANKRD5 | RP5-839B4.5, | ankyrin repeat | 63926 | GATACCTTGGGTTGAATACACTTTT (SEQ ID NO: 94) |
| | | FLJ21669, | domain 5 | | GAATACACTTTTAACAGCCCTAGAT (SEQ ID NO: 95) |
| | | dJ839B4.6 | | | TAACAGCCCTAGATCGATAGCCCTA (SEQ ID NO: 96) |
| | | | | | GCCCTAGCCCTAGATAGATAGCCCT (SEQ ID NO: 97) |
| | | | | | AGATAGCCCTAGCCCTAGATAGATA (SEQ ID NO: 98) |
| | | | | | GTTTACATAATTTTGGGCCCCAGAT (SEQ ID NO: 99) |
| | | | | | GGCCCCAGATGGTTAAGTGCTAAAT (SEQ ID NO: 100) |
| | | | | | GGGTAGGCTATCAGGAAGTGCAGGC (SEQ ID NO: 101) |
| | | | | | GGCTGGAAACATGCAGGAGCTGTCC (SEQ ID NO: 102) |
| | | | | | GTCCACAAGTGGAATTTCTTCTTCA (SEQ ID NO: 103) |
| | | | | | GAAACCTCAGTTTTGTTCATACACC (SEQ ID NO: 104) |
| 210974_s_at | AP3D1 | ADTD, hBLVR | adaptor-related | 8943 | CGTGGCCATCCGCGTGAAGGCCATC (SEQ ID NO: 105) |
| | | | protein complex 3, | | TGTCTGCGCTGCTTGACAGTGCACA (SEQ ID NO: 106) |
| | | | delta 1 subunit | | GCAGCACCCAGCGGAACGGGATCTG (SEQ ID NO: 107) |
| | | | | | GCGGAACGGGATCTGTGAGGTGCTG (SEQ ID NO: 108) |
| | | | | | GGATCTGTGAGGTGCTGTACGCTGC (SEQ ID NO: 109) |
| | | | | | CTGCGGGGAGTTCTCAGAGCATCTG (SEQ ID NO: 110) |
| | | | | | CAGGCCGTGTATGTGCAGAACGTGG (SEQ ID NO: 111) |
| | | | | | CAGAACGTGGTCAAGCTCTACGCCT (SEQ ID NO: 112) |
| | | | | | AGTTTGTGCAGAGCGCAGACCTGGA (SEQ ID NO: 113) |
| | | | | | GAAGCTTCAGGCCAAGGACGTGCCT (SEQ ID NO: 114) |
| | | | | | GGCCAAGGACGTGCCTGTGGCAGAG (SEQ ID NO: 115) |
| 205980_s_at | ARHGAP8 | BPGAP1, | Rho GTPase | 23779 | GCCTGTGTCTTCGGGCTGAATTTGA (SEQ ID NO: 116) |
| | /// | FLJ20185, | activating protein 8 /// | 553158 | GTGTCTTCGGGCTGAATTTGATCTG (SEQ ID NO: 117) |
| | LOC553158 | PP610 /// | PRR5-ARHGAP8 | 55615 | TGAATTTGATCTGGCCATCCCAGGG (SEQ ID NO: 118) |
| | /// PRR5 | LOC553158; | fusion /// proline rich | | AGTCCCCTGATGGCAGCCAGAAGAC (SEQ ID NO: 119) |
| | | ARHGAP8 /// | 5 (renal) | | TGATGGCAGCCAGAAGACGTCTCTA (SEQ ID NO: 120) |
| | | ARHGAP8, | | | GGCAGCCAGAAGACGTCTCTAGTGT (SEQ ID NO: 121) |
| | | FLJ20185, | | | GCCAGAAGACGTCTCTAGTGTTGCG (SEQ ID NO: 122) |
| | | PP610, | | | GAAGACGTCTCTAGTGTTGCGAACA (SEQ ID NO: 123) |
| | | PROTOR1 | | | GTCTCTAGTGTTGCGAACACTCTGT (SEQ ID NO: 124) |
| | | | | | TGTGCACTTGTATGTTTTGTAAACT (SEQ ID NO: 125) |
| | | | | | TTGTAAACTTGGCATCTGTAAAAAT (SEQ ID NO: 126) |
| 202091_at | ARL2BP | BART1 | ADP-ribosylation | 23568 | TTTTCTGCTCTGTAGTCCAAAAACA (SEQ ID NO: 127) |
| | | | factor-like 2 binding | | GCATGTCGAATATGTCACCAGCCAC (SEQ ID NO: 128) |
| | | | protein | | CAGCCACTTCATCCTTATGGTGCTG (SEQ ID NO: 129) |
| | | | | | GAAGGCTGCCATGTTGAACTCAGGA (SEQ ID NO: 130) |
| | | | | | TCAAACTCCAGGTAGTACTTGTCCA (SEQ ID NO: 131) |
| | | | | | AGTACTTGTCCATGAAATTTCTCTG (SEQ ID NO: 132) |
| | | | | | TCGTCATCCATGATAATGTCCTCTA (SEQ ID NO: 133) |
| | | | | | ATATCCAACCACAGCATCAAATTCT (SEQ ID NO: 134) |
| | | | | | GAAAGACAGCGCAAAGCTCTCTCCT (SEQ ID NO: 135) |
| | | | | | TTCTAAGGCGTCCATCGCAGTCGCC (SEQ ID NO: 136) |
| | | | | | GGTTAAGGCCTCTCAGCCAAGGCCG (SEQ ID NO: 137) |
| 205020_s_at | ARL4A | ARL4 | ADP-ribosylation | 10124 | GGGAGTCCCTGTACTTATAGTTGCT (SEQ ID NO: 138) |
| | | | factor-like 4A | | GAGGAACTCATTGTCACTTTCAGAA (SEQ ID NO: 139) |
| | | | | | GGTGAACTGAGCTCATCAACTCCTT (SEQ ID NO: 140) |
| | | | | | CAACTCCTTGGCATTTGCAGCCTAC (SEQ ID NO: 141) |
| | | | | | TGCAGCCTACCTGTGCAATCATAGG (SEQ ID NO: 142) |
| | | | | | GGAGTAGGTTTTCTCTGGTCTGATT (SEQ ID NO: 143) |
| | | | | | TCTGCCCTCCTGGATGCTATTAAAG (SEQ ID NO: 144) |
| | | | | | GAACAATCAGATGCCCAACTCTGTT (SEQ ID NO: 145) |
| | | | | | CCAACTCTGTTGCCTTGTGGAAGAT (SEQ ID NO: 146) |
| | | | | | GTGCTTCTTAAAGTGGTCTCTTCTC (SEQ ID NO: 147) |
| | | | | | TTGGTACTACCATTTGGGGAAGCCA (SEQ ID NO: 148) |
| 220597_s_at | ARL6IP4 /// | MGC814, SR- | ADP-ribosylation-like | 51329 | CTTCCTCCAGTGATGGCCGGAAGAA (SEQ ID NO: 149) |
| | OGFOD2 | 25, SRp25 /// | factor 6 interacting | 79676 | GTGATGGCCGGAAGAAGCGGGGGAA (SEQ ID NO: 150) |
| | | DKFZp686H15154, | protein 4 /// 2- | | GAAGCGGGGGAAGTACAAGGACAAG (SEQ ID NO: 151) |
| | | FLJ13491, | oxoglutarate and | | GGCAAGGAGAAGGCGGAAGCACAGC (SEQ ID NO: 152) |
| | | FLJ37501, | iron-dependent | | GGGCGCACCAGGCTTATTAAGGGAG (SEQ ID NO: 153) |
| | | MGC120434, | oxygenase domain | | CGCACCAGGCTTATTAAGGGAGATG (SEQ ID NO: 154) |
| | | MGC120436 | containing 2 | | GATGGCGAGGTCCTAGAGGAAATCG (SEQ ID NO: 155) |
| | | | | | AGGGTGCCAGTGGGAAGCCTGATGG (SEQ ID NO: 156) |
| | | | | | GCCAGTGGGAAGCCTGATGGGTGCT (SEQ ID NO: 157) |
| | | | | | GTGGGAAGCCTGATGGGTGCTGGTG (SEQ ID NO: 158) |
| | | | | | GGAAGCCTGATGGGTGCTGGTGGCC (SEQ ID NO: 159) |
| 217852_s_at | ARL8B | ARL10C, | ADP-ribosylation | 55207 | TGAAGCATGTTGCATACTACCCTGG (SEQ ID NO: 160) |
| | | FLJ10702, Gie1 | factor-like 8B | | TAAACTTGCTACTGTTGTTCAACAC (SEQ ID NO: 161) |
| | | | | | GAATGTGGCAACTTGCTTGTGCCTA (SEQ ID NO: 162) |
| | | | | | GACTCTGTGGGGACTGCATAGGTTT (SEQ ID NO: 163) |
| | | | | | TGTGTCTATACATTGCTTTCCGCAT (SEQ ID NO: 164) |
| | | | | | TGCTTTCCGCATTTCAAGACATCCA (SEQ ID NO: 165) |
| | | | | | AAGACATCCAGACGCTATTACCAAC (SEQ ID NO: 166) |
| | | | | | GACGCTATTACCAACATTTTCCTGT (SEQ ID NO: 167) |
| | | | | | ACATTTTCCTGTGCATTAACCTCTG (SEQ ID NO: 168) |
| | | | | | AGGTGGATGCATTTTTTGTCTGTTT (SEQ ID NO: 169) |
| | | | | | TGTCAGAAATCAGATCAGCCCTCAA (SEQ ID NO: 170) |
| 202024_at | ASNA1 | ARSA-I, ARSA1, | arsA arsenite | 439 | CGTCATCCGCTCAGTCAGCGAACAG (SEQ ID NO: 171) |
| | | MGC3821 | transporter, ATP- | | GGACCCTGAGCAGACAACTTTCATC (SEQ ID NO: 172) |
| | | | binding, homolog 1 | | GCGTATGCATTGCTGAGTTCCTGTC (SEQ ID NO: 173) |
| | | | (bacterial) | | TATAATTGTCAACCAGCTCGTCTTC (SEQ ID NO: 174) |
| | | | | | CCCGAGAAGCCCTGCAAGATGTGTG (SEQ ID NO: 175) |
| | | | | | TGAGGCCCGTCACAAGATCCAGGCC (SEQ ID NO: 176) |
| | | | | | AGACTTCCACATCGTGAAGCTGCCG (SEQ ID NO: 177) |
| | | | | | CCGCTGTTACCCCATGAGGTGCGGG (SEQ ID NO: 178) |
| | | | | | CAGACAAGGTCAACACCTTCTCGGC (SEQ ID NO: 179) |
| | | | | | GGGCAGAGTTTGCACAAAGTCCCCC (SEQ ID NO: 180) |
| | | | | | TGGTGGGGAGCTGTAGTTGCCCCCT (SEQ ID NO: 181) |
| 218857_s_at | ASRGL1 | ALP, ALP1, | asparaginase like 1 | 80150 | GACTCACCCTGTTCCACATAGAACA (SEQ ID NO: 182) |
| | | FLJ22316 | | | AGAAGAGGCTGCGGACCTATCGTTG (SEQ ID NO: 183) |
| | | | | | GTGGCCTCATCGTGGTTAGCAAAAC (SEQ ID NO: 184) |
| | | | | | GGGTGGCAAAGTGGACCTCCACCTC (SEQ ID NO: 185) |
| | | | | | GGCAAGCTGCACTTCGGAATTGATC (SEQ ID NO: 186) |
| | | | | | TGACGATACTACTATCACCGACCTT (SEQ ID NO: 187) |
| | | | | | GACCTTCCCTAAGCCGCTGGAAGAT (SEQ ID NO: 188) |
| | | | | | GAGGTCAAGTACAGTCTCCTCATGA (SEQ ID NO: 189) |
| | | | | | GGAAAAATTGTCCCGTCTGTCACTT (SEQ ID NO: 190) |
| | | | | | TGTCACTTGTTTTGTTGCCTTAATA (SEQ ID NO: 191) |
| | | | | | GGGGCGGGTTCTGAAGCGATGAGAG (SEQ ID NO: 192) |
| 213036_x_at | ATP2A3 | SERCA3 | ATPase, Ca++ | 489 | CACGACACCCATGAGCCGAAAGGAT (SEQ ID NO: 193) |
| | | | transporting, | | TAAGGATGGCTGATTCCCCAAGGGC (SEQ ID NO: 194) |
| | | | ubiquitous | | CTCCCTCCCTGCTGGAGGAACACGT (SEQ ID NO: 195) |
| | | | | | TTTCTCTTAACTCAGCTTTTGCTAC (SEQ ID NO: 196) |
| | | | | | GAAACCTGCACCAAGGATTGTCCCT (SEQ ID NO: 197) |
| | | | | | CCCTCCTAGAGCGTGTGCAGACTGA (SEQ ID NO: 198) |
| | | | | | TAAATCAAGACTCACATCCCTTTCC (SEQ ID NO: 199) |
| | | | | | CAGCCTGCCTTGCAGACCAATGGGC (SEQ ID NO: 200) |
| | | | | | AATGGGCTCCATGTTCTGTAGCCCC (SEQ ID NO: 201) |
| | | | | | GTCTTAGCAGGAGCGGGCCCTGTAC (SEQ ID NO: 202) |
| | | | | | CCTGCTGACCTGCTGTTTGGTAGAG (SEQ ID NO: 203) |
| 201322_at | ATP5B | ATPMB, ATPSB, | ATP synthase, H+ | 506 | TTGGATGCTACCACTGTACTGTCGC (SEQ ID NO: 204) |
| | | MGC5231 | transporting, | | TCGCGTGCCATTGCTGAGCTGGGCA (SEQ ID NO: 205) |
| | | | mitochondrial F1 | | AGCTGTGGATCCTCTAGACTCCACC (SEQ ID NO: 206) |
| | | | complex, beta | | GTGAGCATTACGATGTTGCCCGTGG (SEQ ID NO: 207) |
| | | | polypeptide | | TCCTGCAGGACTACAAATCCCTCCA (SEQ ID NO: 208) |
| | | | | | GAAGACAAGTTGACCGTGTCCCGTG (SEQ ID NO: 209) |
| | | | | | TTGTCTCAGCCATTCCAGGTTGCTG (SEQ ID NO: 210) |
| | | | | | GAGGTCTTCACAGGTCATATGGGGA (SEQ ID NO: 211) |
| | | | | | GAAGCTGGTACCCCTGAAGGAGACC (SEQ ID NO: 212) |
| | | | | | ACAGGCCTTCTATATGGTGGGACCC (SEQ ID NO: 213) |
| | | | | | TCGTGAGGGGTCTTTGTCCTCTGTA (SEQ ID NO: 214) |
| 207507_s_at | ATP5G3 | MGC125738, P3 | ATP synthase, H+ | 518 | CGCGCAGTCCCGAAGAGTGTAAGAT (SEQ ID NO: 215) |
| | | | transporting, | | CCTCTCTGATCCGAGCTGGATCCAG (SEQ ID NO: 216) |
| | | | mitochondrial F0 | | GCATACAGACCAATTTCTGCATCAG (SEQ ID NO: 217) |
| | | | complex, subunit C3 | | TGCATCAGTGTTATCTCGACCAGAG (SEQ ID NO: 218) |
| | | | (subunit 9) | | ATCTCGACCAGAGGCTAGTAGGACT (SEQ ID NO: 219) |
| | | | | | TGGGGCCCAGAATGGTGTGTCTCAG (SEQ ID NO: 220) |
| | | | | | GTGTCTCAGCTAATCCAAAGGGAGT (SEQ ID NO: 221) |
| | | | | | TTGTTTGATGGTTGCTTTCTTGATT (SEQ ID NO: 222) |
| | | | | | ACTGCTTGACATGTTGGCATTCATA (SEQ ID NO: 223) |
| | | | | | TGTGTATCTTACTGTGACTCCGAAA (SEQ ID NO: 224) |
| | | | | | AAAACTGTAGTATTGGTGTCATGGG (SEQ ID NO: 225) |
| 200818_at | ATP5O | ATPO, OSCP | ATP synthase, H+ | 539 | GAAGGAACCCAAAGTGGCTGCTTCT (SEQ ID NO: 226) |
| | | | transporting, | | GAATCCCTATGTGAAGCGTTCCATT (SEQ ID NO: 227) |
| | | | mitochondrial F1 | | CCTCACTACCAACCTGATCAATTTG (SEQ ID NO: 228) |
| | | | complex, O subunit | | CAATACCCAAGGAGTCGTTTCTGCC (SEQ ID NO: 229) |
| | | | (oligomycin sensitivity | | CTACCATGATGAGTGTCCATCGCGG (SEQ ID NO: 230) |
| | | | conferring protein) | | GTGTCCATCGCGGAGAGGTACCTTG (SEQ ID NO: 231) |
| | | | | | GAGGTACCTTGCACAGTGACCTCTG (SEQ ID NO: 232) |
| | | | | | ATCTCCTTTAGAAGAAGCCACACTC (SEQ ID NO: 233) |
| | | | | | AGCCACACTCTCTGAATTAAAAACT (SEQ ID NO: 234) |
| | | | | | GGAGGCTAAGACTGATCCGTCAATC (SEQ ID NO: 235) |
| | | | | | GACTGATCCGTCAATCTTGGGTGGA (SEQ ID NO: 236) |
| 201089_at | ATP6V1B2 | ATP6B1B2, | ATPase, H+ | 526 | CTTTCCTGAGTGTTTGCTTTCGGTT (SEQ ID NO: 237) |
| | | ATP6B2, HO57, | transporting, | | GCTTTCGGTTTGTTGGTATGCCTGT (SEQ ID NO: 238) |
| | | VATB, VPP3, | lysosomal 56/58 kDa, | | TCTTTTTGCTCTTTCCTATATAATG (SEQ ID NO: 239) |
| | | Vma2 | V1 subunit B2 | | TGGGATTCCTGTGTCTTACAGCTCT (SEQ ID NO: 240) |
| | | | | | GCAACTTTTTGCACAGCTGGTATCT (SEQ ID NO: 241) |
| | | | | | TCTGGTAGCAGTGAGACCCCTTGTC (SEQ ID NO: 242) |
| | | | | | GGTGATCCTTACTGGGTTTCCAAGC (SEQ ID NO: 243) |
| | | | | | AAACCTACAGCCTTTCTACATTCTG (SEQ ID NO: 244) |
| | | | | | AGAGAGCAGCCTTTGAAAATCCCCA (SEQ ID NO: 245) |
| | | | | | GAAAATCCCCAATTTGGTTCTGCTT (SEQ ID NO: 246) |
| | | | | | CCTCTCTCTACCTTTTCAGGGTAAT (SEQ ID NO: 247) |
| 215608_at | AU144378* | | | | AAAGTGACTTGCCTGGAATGATACA (SEQ ID NO: 248) |
| | | | | | GCCTGGAATGATACAGCCACCGAGC (SEQ ID NO: 249) |
| | | | | | CCACCGAGCAGGAGGCAAGGAAAGC (SEQ ID NO: 250) |
| | | | | | GCAAGGAAAGCCAGCTCTGTCTAAC (SEQ ID NO: 251) |
| | | | | | AAGCCAGCTCTGTCTAACTGCTGCT (SEQ ID NO: 252) |
| | | | | | AGCTCTGTCTAACTGCTGCTGAGTA (SEQ ID NO: 253) |
| | | | | | GTCTAACTGCTGCTGAGTACTTTGT (SEQ ID NO: 254) |
| | | | | | ACTGCTGCTGAGTACTTTGTAAAAG (SEQ ID NO: 255) |
| | | | | | ACAAAAAATGTGATCCAGAACTGTG (SEQ ID NO: 256) |
| | | | | | TTCGAACCCAGCCTTTACAATGTAA (SEQ ID NO: 257) |
| | | | | | TTACAATGTAATGAAACCCTGCCTC (SEQ ID NO: 258) |
| 218580_x_at | AURKAIP1 | AIP, AKIP, | aurora kinase A | 54998 | GGAGCTGGAGGAGATGCTGGTCCCC (SEQ ID NO: 259) |
| | | FLJ20608 | interacting protein 1 | | CTGCCCAGACTGGATACCGGGACCG (SEQ ID NO: 260) |
| | | | | | TGTCCGCCCAGCCAGATAGGGGAAG (SEQ ID NO: 261) |
| | | | | | ATGAAGGCGTCGCGGATGCGCCTCA (SEQ ID NO: 262) |
| | | | | | AAAAACGTGCTGAAGATCCGCCGGC (SEQ ID NO: 263) |
| | | | | | TGAACCACCACAAGTACCGGAAGCT (SEQ ID NO: 264) |
| | | | | | GACGCGGTTCCTGCGGAGGAAGGTC (SEQ ID NO: 265) |
| | | | | | GGACGCCTGAGACGCAAGCAGATCA (SEQ ID NO: 266) |
| | | | | | ACCTGAGGCGCATCTGGCTGAAGGC (SEQ ID NO: 267) |
| | | | | | CCCCCAAGATCTACCTGCGGGGCAA (SEQ ID NO: 268) |
| | | | | | CGTTGCTGCTGTGATCCGTAGTAAT (SEQ ID NO: 269) |
| 219326_s_at | B3GNT2 | B3GN-T1, B3GN- | UDP-GlcNAc:betaGal | 10678 | GACGTTTATACTGGAATGTGCCTTC (SEQ ID NO: 270) |
| | | T2, B3GNT, | beta-1,3-N- | | AATGTGCCTTCAGAAACTCGGCCTC (SEQ ID NO: 271) |
| | | B3GNT-2, | acetylglucosaminyltransferase 2 | | CTCGGCCTCGTTCCAGAGAAACACA (SEQ ID NO: 272) |
| | | B3GNT1, | | | AACATCTGCTCCTATGTAGATCTGA (SEQ ID NO: 273) |
| | | BETA3GNT | | | TGGTCTCAGTTGCAGAGTGCTCATT (SEQ ID NO: 274) |
| | | | | | GAGTGATACCCATTTGAGGGCCTCT (SEQ ID NO: 275) |
| | | | | | TTGAGGGCCTCTAAACCCTTCAATT (SEQ ID NO: 276) |
| | | | | | GTTCTGATCTTACCGGCTAGTGGTC (SEQ ID NO: 277) |
| | | | | | TAAAAAACTTGTACCCTCTTATCTG (SEQ ID NO: 278) |
| | | | | | GTACCCTCTTATCTGAAATCCTGTT (SEQ ID NO: 279) |
| | | | | | CTTCTATAATATTCCTACTTCCCAT (SEQ ID NO: 280) |
| 91920_at | BCAN | RP11-66D17.1, | brevican | 63827 | AGCCAGCAGCCCAGGAGGAGTCACT (SEQ ID NO: 281) |
| | | BEHAB, CSPG7, | | | AGCCCAGGAGGAGTCACTCTCCCAG (SEQ ID NO: 282) |
| | | MGC13038 | | | CAGCAAGGGCAGTCCTGCAGCCTGG (SEQ ID NO: 283) |
| | | | | | AGTCCTGCAGCCTGGTGCATCACCA (SEQ ID NO: 284) |
| | | | | | AGCCTGGTGCATCACCACTTCCTGA (SEQ ID NO: 285) |
| | | | | | GGTGCATCACCACTTCCTGATGGAG (SEQ ID NO: 286) |
| | | | | | TTCCTGATGGAGAGTCAGAAGCTTC (SEQ ID NO: 287) |
| | | | | | GAGAGTCAGAAGCTTCCAGGCCTCC (SEQ ID NO: 288) |
| | | | | | CTTCCAGGCCTCCAAGGGTCCATGG (SEQ ID NO: 289) |
| | | | | | TCCAAGGGTCCATGGACCACCTACT (SEQ ID NO: 290) |
| | | | | | GTCCATGGACCACCTACTGAGACTC (SEQ ID NO: 291) |
| | | | | | GAGAGGAACCTAGCATCCCCATCAC (SEQ ID NO: 292) |
| | | | | | CATCACCTTCCACTCTGGTTGAGGC (SEQ ID NO: 293) |
| | | | | | CCACTCTGGTTGAGGCAAGAGAGGT (SEQ ID NO: 294) |
| | | | | | GAGGCAACTGGTGGTCCTGAGCTAT (SEQ ID NO: 295) |
| | | | | | AGCTATCTGGGGTCCCTCGAGGAGA (SEQ ID NO: 296) |
| 213156_at | BG251521* | | Homo sapiens, clone | | ACACACTTCCTATATGTATCCACAT (SEQ ID NO: 297) |
| | | | IMAGE: 4214654, | | ATCAGATGCTCATTACAAATCCAGA (SEQ ID NO: 298) |
| | | | mRNA | | TTGGACTCTTGCAACAGATCACAAA (SEQ ID NO: 299) |
| | | | | | GACTTTGGTCATTTCTGTATGTTCA (SEQ ID NO: 300) |
| | | | | | GGGCGAAACCATCTTGATCCGATGC (SEQ ID NO: 301) |
| | | | | | GATCCGATGCGAATGCAGTAATGTT (SEQ ID NO: 302) |
| | | | | | GTTCTATATACCATTTCATCAGTTA (SEQ ID NO: 303) |
| | | | | | GTTATTTCTTTTAGTCATGTTGATT (SEQ ID NO: 304) |
| | | | | | ACTACAGTGAAAGCCCTTTTCAGTG (SEQ ID NO: 305) |
| | | | | | TGGCCTGACTCAGAACTACCATGTG (SEQ ID NO: 306) |
| | | | | | GAAAATCAGTACTCTCTTAATGTTT (SEQ ID NO: 307) |
| 202592_at | BLOC1S1 | BLOS1, | biogenesis of | 2647 | TCCCGCCTCCTAAAAGAACACCAGG (SEQ ID NO: 308) |
| | | FLJ39337, | lysosome-related | | GGAGGCGAGAGGCTATCACTGCAGC (SEQ ID NO: 309) |
| | | FLJ97089, | organelles complex- | | CGACCTGCCTGACAGAAGCTTTGGT (SEQ ID NO: 310) |
| | | GCN5L1, | 1, subunit 1 | | GGTGTGGCCCAGGCCTACATGAACC (SEQ ID NO: 311) |
| | | MGC87455, | | | TGAAGACCCTACAGGTCCAGGCTGC (SEQ ID NO: 312) |
| | | MICoA, RT14 | | | CAGGCTGCCCAATTTGCCAAGCAGA (SEQ ID NO: 313) |
| | | | | | GCCAAGCAGACAGGCCAGTGGATCG (SEQ ID NO: 314) |
| | | | | | GGTGGAGAACTTCAACCAGGCACTC (SEQ ID NO: 315) |
| | | | | | TGTGGAGAACTGGGCTCGGAGCATC (SEQ ID NO: 316) |
| | | | | | CACCATTGCCACTGCACTGGAATAT (SEQ ID NO: 317) |
| | | | | | CTACCTCACCCGCAGGGGGAAGGAG (SEQ ID NO: 318) |
| 218220_at | C12orf10 | MST024, | chromosome 12 open | 60314 | CACGAGTTGCTCGACTTAATCCTAC (SEQ ID NO: 319) |
| | | MSTP024, MYG, | reading frame 10 | | TCAAGCGTGCAATGGATCTGGTTCA (SEQ ID NO: 320) |
| | | MYG1 | | | GATTAGATTTCTACCAACACAGCTG (SEQ ID NO: 321) |
| | | | | | CAGCGATTCCAGGTGGACCCAAGTG (SEQ ID NO: 322) |
| | | | | | ACTGGCGAAAGGTGCATGTCCCTGG (SEQ ID NO: 323) |
| | | | | | ATGTCCCTGGAAGGAGCATCTCTAC (SEQ ID NO: 324) |
| | | | | | CATCTCTACCACCTGGAATCTGGGC (SEQ ID NO: 325) |
| | | | | | CCATCTTCTTTGTTATCTACACTGA (SEQ ID NO: 326) |
| | | | | | GGACCAGGTCAGTGGGATCCCTGGC (SEQ ID NO: 327) |
| | | | | | GTCCATGCAAGCGGCTTCATTGGCG (SEQ ID NO: 328) |
| | | | | | CCGACCCGAGAGGGTGCCTTGAGCA (SEQ ID NO: 329) |
| 221777_at | C12orf52 | FLJ14827 | chromosome 12 open | 84934 | GTTAGCATTTCAGTGCCATCTACCC (SEQ ID NO: 330) |
| | | | reading frame 52 | | GCCATCTACCCCACGACGAGGTGGG (SEQ ID NO: 331) |
| | | | | | AGCCCCCTTGGAAATGATACTCTTT (SEQ ID NO: 332) |
| | | | | | GATACTCTTTCATCAGGGTTGCCTA (SEQ ID NO: 333) |
| | | | | | CTATGGGGCCACGGCGACAGGTATG (SEQ ID NO: 334) |
| | | | | | ATGGCCCCTTGCCAGGGTAGGAGGA (SEQ ID NO: 335) |
| | | | | | TCCCTTTATCCTGACAATCTCTAGT (SEQ ID NO: 336) |
| | | | | | AATCTCTAGTCGATTCTTGCCTTTT (SEQ ID NO: 337) |
| | | | | | CCTTTTTCTCCCGATTGCGGATTTG (SEQ ID NO: 338) |
| | | | | | ATTTGGGGGCCACCTCTAAGATGCC (SEQ ID NO: 339) |
| | | | | | AAATTAGTGCCAACCCAGGGGCCTG (SEQ ID NO: 340) |
| 214296_x_at | C19orf36 | IMAGE: 4215339 | chromosome 19 open | 113177 | ACGAGGGGTCGTGGATGTGGCCACA (SEQ ID NO: 341) |
| | | | reading frame 36 | | GTGGCCACACATAGGACCACACGTC (SEQ ID NO: 342) |
| | | | | | ACCACACGTCCCAGCTGGGAGGAGA (SEQ ID NO: 343) |
| | | | | | GTGGGGGACATGGAGAGCTGAGGCA (SEQ ID NO: 344) |
| | | | | | TCGGCGCAGTGGAAGTCAGCTGTCC (SEQ ID NO: 345) |
| | | | | | CTGGTATCGCCAGCCTTAAGGTGTC (SEQ ID NO: 346) |
| | | | | | ACTTGGCCAACCTGACCTTGGAAGA (SEQ ID NO: 347) |
| | | | | | GGAAGATGCTGCTGAGTGTCTCAAG (SEQ ID NO: 348) |
| | | | | | AGCAGCACTGACAGCAGCTGGGCCT (SEQ ID NO: 349) |
| | | | | | GGGCGGAGACTCAGCTGGACAGCCC (SEQ ID NO: 350) |
| | | | | | GAGGGAGGGAATGGGGGTGGGCTGT (SEQ ID NO: 351) |
| 219439_at | C1GALT1 | C1GALT, T- | core 1 synthase, | 56913 | TCTGACATAGAACACTGGAATCCCA (SEQ ID NO: 352) |
| | | synthase | glycoprotein-N- | | GGAATCCCAGTGAGGAATTCTAAGT (SEQ ID NO: 353) |
| | | | acetylgalactosamine | | GAACATTCCTTATAGAAACCTTTCA (SEQ ID NO: 354) |
| | | | 3-beta- | | AATGGTGGCCAGGTAGAGGAACTAG (SEQ ID NO: 355) |
| | | | galactosyltransferase, 1 | | GACCATCTGTGTTATTGTCACTGAG (SEQ ID NO: 355) |
| | | | | | ATTTACTAAAACTACACTGCACCAT (SEQ ID NO: 357) |
| | | | | | AACTACACTGCACCATGTTAGTAAT (SEQ ID NO: 358) |
| | | | | | GAAATATTGTTGCTCAGTGTTGTTA (SEQ ID NO: 359) |
| | | | | | TATGCTATAAATGATACCCCCCTAC (SEQ ID NO: 360) |
| | | | | | TAATTGGTAGTATTTCTTCCAGAAG (SEQ ID NO: 361) |
| | | | | | GACTGGCACTTACCCTGAAGTGCAT (SEQ ID NO: 362) |
| 212005_at | C1orf144 | DKFZp566C0424, | chromosome 1 open | 26099 | TTCCCATGAGCCACTACTCTGATGT (SEQ ID NO: 363) |
| | | MGC70432 | reading frame 144 | | TCCCATGAGCCACTACTCTGATGTC (SEQ ID NO: 364) |
| | | | | | CCATGAGCCACTACTCTGATGTCAG (SEQ ID NO: 365) |
| | | | | | GTCAGCCTATAACCAAAGGAGCTGG (SEQ ID NO: 366) |
| | | | | | GCCTATAACCAAAGGAGCTGGGGGG (SEQ ID NO: 367) |
| | | | | | TTTCTCAGCCCACTCAATCAGGGTG (SEQ ID NO: 368) |
| | | | | | CTCAGCCCACTCAATCAGGGTGCTC (SEQ ID NO: 369) |
| | | | | | AGCCTCCCTGGTAAGCAGAGACTCA (SEQ ID NO: 370) |
| | | | | | CCTCCCTGGTAAGCAGAGACTCAAG (SEQ ID NO: 371) |
| | | | | | GAGACTCAAGAAACCTCTGGGGTCC (SEQ ID NO: 372) |
| | | | | | GACTCAAGAAACCTCTGGGGTCCTG (SEQ ID NO: 373) |
| 210652_s_at | C1orf34 | C1orf34, DEME- | chromosome 1 open | 22996 | CACCTGTAAGTCTTTGTCTTCACCT (SEQ ID NO: 374) |
| | | 6, KIAA0452 | reading frame 34 | | TGTCTTCACCTATTATCATCTGGAG (SEQ ID NO: 375) |
| | | | | | GCTTGTGTGACGGAAGGTCCTTCAG (SEQ ID NO: 376) |
| | | | | | GAGGACCTGAGGAATGCCTGGGAGA (SEQ ID NO: 377) |
| | | | | | AAGCCTCAGGCTTCAATGCTTCTGG (SEQ ID NO: 378) |
| | | | | | GCTTCTGGGGTTGGGCATGAGGATG (SEQ ID NO: 379) |
| | | | | | TGGAAGAGCTGTGTAGCCCTTTCTA (SEQ ID NO: 380) |
| | | | | | GGCAGGAGGAGGTTCTCAGAATCCA (SEQ ID NO: 381) |
| | | | | | AGTCTGTATCTTTGCTGTATGCCAA (SEQ ID NO: 382) |
| | | | | | TAAAAATCTTCTGTACTTCACTCCA (SEQ ID NO: 383) |
| | | | | | ACTTCACTCCAAGGTACATTTGCTT (SEQ ID NO: 384) |
| 222301_at | C1orf61 | CROC4; | Chromosome 1 open | 10485 | CCTATTTAGATCCGTCAGCCCACGG (SEQ ID NO: 385) |
| | | FLJ38303; RP11- | reading frame 61 | | CAAGCAGTCATTCCCTGTTATACAA (SEQ ID NO: 386) |
| | | 139I14.3 | | | GTCATTCCCTGTTATACAAGCAAGA (SEQ ID NO: 387) |
| | | | | | AGACAGCAATGGGGCCTTTAGGCCT (SEQ ID NO: 388) |
| | | | | | CAGGCTGCGGCTGGCATATGGAGGT (SEQ ID NO: 389) |
| | | | | | ATTGGCTGCCATATTGTACCTCAGA (SEQ ID NO: 390) |
| | | | | | GTACCTCAGAGCACTAGCTCCTATC (SEQ ID NO: 391) |
| | | | | | CACTAGCTCCTATCTGGATTTTCCC (SEQ ID NO: 392) |
| | | | | | GCGGGGTTCCTAGAGGGTGGGAGAT (SEQ ID NO: 393) |
| | | | | | GAGATGGGTCTCCAGGGATTTATCC (SEQ ID NO: 394) |
| | | | | | CCTTTCCCCTGATATCTATCCAATA (SEQ ID NO: 395) |
| 219054_at | C5orf23 | CSGalNAcT-2, | chromosome 5 open | 79614 | GACAACAATGAAGTAGCCCCTGAAC (SEQ ID NO: 396) |
| | | DKFZp686H13226, | reading frame 23 | | GTAGCCCCTGAACAGCATGGAGTTG (SEQ ID NO: 397) |
| | | FLJ43310, | | | GAGTTGCTGTGAGTTTGTTCGTTGC (SEQ ID NO: 398) |
| | | GALNACT-2, | | | GTTCGTTGCAGACCTTTGTGTTGGG (SEQ ID NO: 399) |
| | | GALNACT2, | | | GGTCCTGGGAATCTGAGCTTTGTTC (SEQ ID NO: 400) |
| | | MGC40204, | | | CTTTGTTCCCTGTGCATGGTGGATA (SEQ ID NO: 401) |
| | | PRO0082 | | | GGGATAGACCTTGTGACAGACCAAT (SEQ ID NO: 402) |
| | | | | | GACAGACCAATTCTGTGACCCCTGT (SEQ ID NO: 403) |
| | | | | | TGACCCCTGTCTTCTGGGTCACATT (SEQ ID NO: 404) |
| | | | | | AAATGTGTATGTGTCCTTGTAAATG (SEQ ID NO: 405) |
| | | | | | GCAAGAATGCCACGTACTCAGAGTA (SEQ ID NO: 406) |
| 39817_s_at | C6orf108 | RCL, RP3- | chromosome 6 open | 10591 | AGTGACACAGCCATCCTTGGGTGTA (SEQ ID NO: 407) |
| | | 330M21.3, | reading frame 108 | | CACAGCCATCCTTGGGTGTAGGCTA (SEQ ID NO: 408) |
| | | dJ330M21.3 | | | AGTCTGGCCGCGTGCTTTCGGCCAT (SEQ ID NO: 409) |
| | | | | | GTCTGGCCGCGTGCTTTCGGCCATG (SEQ ID NO: 410) |
| | | | | | TGGCCGCGTGCTTTCGGCCATGATC (SEQ ID NO: 411) |
| | | | | | GGCCGCGTGCTTTCGGCCATGATCC (SEQ ID NO: 412) |
| | | | | | TGCTTTCGGCCATGATCCGGGGAGC (SEQ ID NO: 413) |
| | | | | | TTTCGGCCATGATCCGGGGAGCAGC (SEQ ID NO: 414) |
| | | | | | ATCCGGGGAGCAGCAGATGGCTCTC (SEQ ID NO: 415) |
| | | | | | GCAGCAGATGGCTCTCGGTTCCAGG (SEQ ID NO: 416) |
| | | | | | CAGCAGATGGCTCTCGGTTCCAGGT (SEQ ID NO: 417) |
| | | | | | AGCAGATGGCTCTCGGTTCCAGGTG (SEQ ID NO: 418) |
| | | | | | ATACTTCGAGGCTGATCCTCCAGGG (SEQ ID NO: 419) |
| | | | | | TTCGAGGCTGATCCTCCAGGGCAGG (SEQ ID NO: 420) |
| | | | | | TCGAGGCTGATCCTCCAGGGCAGGT (SEQ ID NO: 421) |
| | | | | | ATCCTCCAGGGCAGGTGGCTGCCTC (SEQ ID NO: 422) |
| 221100_at | C6orf15 | DADB-141O4.4, | chromosome 6 open | 29113 | CATCCACAGGGTTCTGCCTGATCAC (SEQ ID NO: 423) |
| | | STG | reading frame 15 | | TGATCACCCCTGGGGTACCCTGAAT (SEQ ID NO: 424) |
| | | | | | TACCCTGAATCCCAGTGTGTCCTGG (SEQ ID NO: 425) |
| | | | | | TGGTTGGGGAACGAGGCCCATGCCA (SEQ ID NO: 426) |
| | | | | | TCAATAATCAACCCCCAGGTACCAG (SEQ ID NO: 427) |
| | | | | | CAGGTATCAATAACCCATTTCCTCC (SEQ ID NO: 428) |
| | | | | | CTGGCTCTTCTTGGAACATCCCAGC (SEQ ID NO: 429) |
| | | | | | CTCCAAGCCCTAGGTTGCAGTGGGG (SEQ ID NO: 430) |
| | | | | | TTGGGAGTTAGAGTCCTGCTCCCGC (SEQ ID NO: 431) |
| | | | | | CTTGCTGTGTGGGCTCAATCCAGGC (SEQ ID NO: 432) |
| | | | | | TCCAGGCCCTGTTAACATGTTTCCA (SEQ ID NO: 433) |
| 219655_at | C7orf10 | DERP13, | chromosome 7 open | 79783 | GCAGAACCTCAGAACGCTGTCTCTG (SEQ ID NO: 434) |
| | | FLJ11808, | reading frame 10 | | GGGTATTACACAATGGCCTCGTTAT (SEQ ID NO: 435) |
| | | ORF19 | | | GATGGAGCATCCAACTGTGGGGAAG (SEQ ID NO: 436) |
| | | | | | CACACAACGCACATCCTGAAGGAGG (SEQ ID NO: 437) |
| | | | | | CGATGACAGGGCCATCGGGGAGCTG (SEQ ID NO: 438) |
| | | | | | TGCTCAGCGCTGGAGTGGTGGACCA (SEQ ID NO: 439) |
| | | | | | GGAAAAGGGCTCTTCCTCATAACCT (SEQ ID NO: 440) |
| | | | | | ACCTCGATCCGAATACACTGGCAAA (SEQ ID NO: 441) |
| | | | | | TCCCCAGTTCTGATACCACTAAGAA (SEQ ID NO: 442) |
| | | | | | AACTCCAGATTTCTTACATGGCATC (SEQ ID NO: 443) |
| | | | | | ACATGGCATCTCCAGAATGGCTCTG (SEQ ID NO: 444) |
| 219060_at | C8orf32 | FLJ10204 | chromosome 8 open | 55093 | AAGCTATCCTTTCATCGAGGACAGC (SEQ ID NO: 445) |
| | | | reading frame 32 | | ACATTATGGTACAGTTGGCTTGGAA (SEQ ID NO: 446) |
| | | | | | GGCTTGGAATTATGTCTTTCTCTTT (SEQ ID NO: 447) |
| | | | | | GTGCGTACTTGCTAAGATATTCCTG (SEQ ID NO: 448) |
| | | | | | GATATTCCTGTGGCTCATGCGTTAC (SEQ ID NO: 449) |
| | | | | | GCGTTACAACACGAGGACTTAAGCC (SEQ ID NO: 450) |
| | | | | | AAGCCAGTAATCGTTTTTGTTCAGA (SEQ ID NO: 451) |
| | | | | | AGAGGTGTGGAGGTAGAGCCAGCCC (SEQ ID NO: 452) |
| | | | | | TGTCTCTCCAGCTACATTGTAAGTT (SEQ ID NO: 453) |
| | | | | | AAATGCCCATAAAAGGTGCCCATAA (SEQ ID NO: 454) |
| | | | | | AGCCCTCTCAGACTTGAGCGTTAAT (SEQ ID NO: 455) |
| 220038_at | C8orf44 /// | FLJ11267 /// | serum/glucocorticoid | 56260 | TTTCCCTCTAAGTTTACACTAACAT (SEQ ID NO: 456) |
| | SGK3 | CISK, | regulated kinase | 23678 | GTTTACACTAACATCTACCCAAGAT (SEQ ID NO: 457) |
| | | DKFZp781N0293, | family, member 3 /// | | ACATCTACCCAAGATAGACTGTTTT (SEQ ID NO: 458) |
| | | SGK2, SGKL | chromosome 8 open | | ACAGTCAATTTCAGTTCAGCTAACA (SEQ ID NO: 459) |
| | | | reading frame 44 | | TTGTAACTCTTTGCTATGGCTTTTG (SEQ ID NO: 460) |
| | | | | | GCTATGGCTTTTGTTATCACACCAA (SEQ ID NO: 461) |
| | | | | | GTTATCACACCAAAACTATGCAATT (SEQ ID NO: 462) |
| | | | | | TATGCAATTGGTACATGGTTGTTTA (SEQ ID NO: 463) |
| | | | | | GAAACCGTATTTTTCCATGATAAAT (SEQ ID NO: 464) |
| | | | | | GAAATATTTGGTTCATGGTATGATC (SEQ ID NO: 465) |
| | | | | | CATAATTAACACATTGGCTGCTAGT (SEQ ID NO: 466) |
| 205308_at | C8orf70 | C8orf70, CGI-62 | chromosome 8 open | 51101 | GCAAAACTGTTGTAGGTGTTCCTTC (SEQ ID NO: 467) |
| | | | reading frame 70 | | AAGTGTCTTCAAGTAGCAGCTCTTT (SEQ ID NO: 468) |
| | | | | | AACAAACTTCAGACCTTGTCTCCCT (SEQ ID NO: 469) |
| | | | | | ATAAAGGGATAGCAGCCCCTCATGC (SEQ ID NO: 470) |
| | | | | | GCAGGAGCTAATGTCAAACCCCGAA (SEQ ID NO: 471) |
| | | | | | GAAATTCCACACCACCTAGTTTGGC (SEQ ID NO: 472) |
| | | | | | GCCCCAGGTGTGCTTACAAACAAAA (SEQ ID NO: 473) |
| | | | | | GAGAGCTACATAGCCAGGCCAGATG (SEQ ID NO: 474) |
| | | | | | AGATGGGGACTGTGCATCTTCCCTT (SEQ ID NO: 475) |
| | | | | | GGACATTCACCTGGAAACTTACCAA (SEQ ID NO: 476) |
| | | | | | GCTTGGACAGCTAGAGCACATCCTC (SEQ ID NO: 477) |
| 64408_s_at | CALML4 | MGC4809, NY- | calmodulin-like 4 | 91860 | GAAAGAAATTCTTCTAGCCATGTTG (SEQ ID NO: 478) |
| | | BR-20 | | | ATTCTTCTAGCCATGTTGATGGTGG (SEQ ID NO: 479) |
| | | | | | CTTCTAGCCATGTTGATGGTGGACA (SEQ ID NO: 480) |
| | | | | | CTAGCCATGTTGATGGTGGACAAGG (SEQ ID NO: 481) |
| | | | | | CGGTCAAAACTCACGAGTCTGGGGG (SEQ ID NO: 482) |
| | | | | | GGTCAAAACTCACGAGTCTGGGGGA (SEQ ID NO: 483) |
| | | | | | GATCACCCTTCCTGGACGGGACTAT (SEQ ID NO: 484) |
| | | | | | CACCCTTCCTGGACGGGACTATTGA (SEQ ID NO: 485) |
| | | | | | TTCCTGGACGGGACTATTGAAGGAG (SEQ ID NO: 486) |
| | | | | | CTCCCCTGGGCCTGAAAACTTGGAG (SEQ ID NO: 487) |
| | | | | | CCTGGGCCTGAAAACTTGGAGCAAT (SEQ ID NO: 488) |
| | | | | | CTGGGCCTGAAAACTTGGAGCAATT (SEQ ID NO: 489) |
| | | | | | AACACAGTGGCAAGACAACATTACC (SEQ ID NO: 490) |
| | | | | | AACATTACCCAACTATAGAAGAGAG (SEQ ID NO: 491) |
| | | | | | TACCCAACTATAGAAGAGAGGCTAA (SEQ ID NO: 492) |
| | | | | | ACCCAACTATAGAAGAGAGGCTAAC (SEQ ID NO: 493) |
| 212710_at | CAMSAP1 | RP11-100C15.2, | calmodulin regulated | 157922 | AACTCTGTTTATCAATGTCTCCTAT (SEQ ID NO: 494) |
| | | DKFZp434F195, | spectrin-associated | | TCCTATATTCAAAGCCCCTGCAGTG (SEQ ID NO: 495) |
| | | DKFZp434G2311, | protein 1 | | GCAGTGGTATTTGTGTCTCGTTGCA (SEQ ID NO: 496) |
| | | FLJ31228, | | | CAACTTTCGTTGTGTCTCAGAATTC (SEQ ID NO: 497) |
| | | MGC163452, | | | TAAGACAAGCTGCTCTACCCATTTA (SEQ ID NO: 498) |
| | | PRO2405, | | | ACTTGCGTGAGTCCCATTATTTTAC (SEQ ID NO: 499) |
| | | bA100C15.1 | | | TGTGCAGATACGTTCTACCCACACG (SEQ ID NO: 500) |
| | | | | | GCGTGCACATGGCTGTGTGCGTGCA (SEQ ID NO: 501) |
| | | | | | AGATGGACTGCTTTCCATGTGTCCT (SEQ ID NO: 502) |
| | | | | | CTCTTAGCAGACTTCAGGCCCAAAC (SEQ ID NO: 503) |
| | | | | | TCTAGGTTATACTTTTACCTCCCTG (SEQ ID NO: 504) |
| 212765_at | CAMSAP1L1 | RP11-93N17.1, | calmodulin regulated | 23271 | GAGTTCATTGCTCTCAGTATAAGAT (SEQ ID NO: 505) |
| | | KIAA1078, | spectrin-associated | | AAGTCTGAGGATTTTCGTCAACCTT (SEQ ID NO: 506) |
| | | MGC150680, | protein 1-like 1 | | CGTCAACCTTACTGAAACACACTGG (SEQ ID NO: 507) |
| | | MGC150681 | | | AACACACTGGTGCTTTCATCATCAG (SEQ ID NO: 508) |
| | | | | | TTGTCGTGGTTACCAGTGCAGCCTG (SEQ ID NO: 509) |
| | | | | | AAATTCTGCTATTTGACACAGCTTT (SEQ ID NO: 510) |
| | | | | | ATGGTTTCATGGACACTGTTGAGCA (SEQ ID NO: 511) |
| | | | | | AGTGTATGGTGTGCTTACCTGTCCA (SEQ ID NO: 512) |
| | | | | | TACCTGTCCACTCTAGAGCATTGCT (SEQ ID NO: 513) |
| | | | | | GAGCATTGCTTACAGGTTTTTTGTT (SEQ ID NO: 514) |
| | | | | | TGCTATTTCCTGGTACAGTGTAGTT (SEQ ID NO: 515) |
| 209970_x_at | CASP1 | ICE, IL1BC, P45 | caspase 1, | 834 | CGAAGGTGATCATCATCCAGGCCTG (SEQ ID NO: 516) |
| | | | apoptosis-related | | ACAGCCCTGGTGTGGTGTGGTTTAA (SEQ ID NO: 517) |
| | | | cysteine peptidase | | GGAAACCTATCTTTACCAACTACAG (SEQ ID NO: 518) |
| | | | (interleukin 1, beta, | | GAAGGATTTTATCGCTTTCTGCTCT (SEQ ID NO: 519) |
| | | | convertase) | | TTTCTGCTCTTCCACACCAGATAAT (SEQ ID NO: 520) |
| | | | | | GAGACATCCCACAATGGGCTCTGTT (SEQ ID NO: 521) |
| | | | | | GAATATGCCTGTTCCTGTGATGTGG (SEQ ID NO: 522) |
| | | | | | TTTCCGCAAGGTTCGATTTTCATTT (SEQ ID NO: 523) |
| | | | | | GATGGTAGAGCGCAGATGCCCACCA (SEQ ID NO: 524) |
| | | | | | TGACAAGATGTTTCTACCTCTTCCC (SEQ ID NO: 525) |
| | | | | | TTCTACCTCTTCCCAGGACATTAAA (SEQ ID NO: 526) |
| 211367_s_at | CASP1 | ICE, IL1BC, P45 | caspase 1, | 834 | GAGCTGAGGTTGACATCACAGGCAT (SEQ ID NO: 527) |
| | | | apoptosis-related | | ATCACAGGCATGACAATGCTGCTAC (SEQ ID NO: 528) |
| | | | cysteine peptidase | | GACAATGCTGCTACAAAATCTGGGG (SEQ ID NO: 529) |
| | | | (interleukin 1, beta, | | AAATCTGGGGTACAGCGTAGATGTG (SEQ ID NO: 530) |
| | | | convertase) | | AAAAAAATCTCACTGCTTCGGACAT (SEQ ID NO: 531) |
| | | | | | TCATGTCTCATGGTATTCGGGAAGG (SEQ ID NO: 532) |
| | | | | | GGTATTCGGGAAGGCATTTGTGGGA (SEQ ID NO: 533) |
| | | | | | GAAGAAACACTCTGAGCAAGTCCCA (SEQ ID NO: 534) |
| | | | | | ACTACAACTCAATGCAATCTTTAAC (SEQ ID NO: 535) |
| | | | | | GTTGAATACCAAGAACTGCCCAAGT (SEQ ID NO: 536) |
| | | | | | ACCGAAGGTGATCATCATCCAGGCC (SEQ ID NO: 537) |
| 211368_s_at | CASP1 | ICE, IL1BC, P45 | caspase 1, | 834 | AATGTTTCTTGGAGACATCCCACAA (SEQ ID NO: 538) |
| | | | apoptosis-related | | GGAGACATCCCACAATGGGCTCTGT (SEQ ID NO: 539) |
| | | | cysteine peptidase | | GGCTCTGTTTTTATTGGAAGACTCA (SEQ ID NO: 540) |
| | | | (interleukin 1, beta, | | GGAAGACTCATTGAACATATGCAAG (SEQ ID NO: 541) |
| | | | convertase) | | TATGCAAGAATATGCCTGTTCCTGT (SEQ ID NO: 542) |
| | | | | | GAGGAAATTTTCCGCAAGGTTCGAT (SEQ ID NO: 543) |
| | | | | | AAGGTTCGATTTTCATTTGAGCAGC (SEQ ID NO: 544) |
| | | | | | TGAGCAGCCAGATGGTAGAGCGCAG (SEQ ID NO: 545) |
| | | | | | GATGGTAGAGCGCAGATGCCCACCA (SEQ ID NO: 546) |
| | | | | | GACTTTGACAAGATGTTTCTACCTC (SEQ ID NO: 547) |
| | | | | | CTACCTCTTCCCAGGACATTAAAAT (SEQ ID NO: 548) |
| 220466_at | CCDC15 | FLJ13215 | coiled-coil domain | 80071 | GGGATTTTCTACCCAGAGACCTGCA (SEQ ID NO: 549) |
| | | | containing 15 | | ATGTTCTCTCCAACGACCAGAATAT (SEQ ID NO: 550) |
| | | | | | TGTCAGGACCAAGATTTTCTACCAA (SEQ ID NO: 551) |
| | | | | | AGAAGACTTTTCTCTGGCAGACTAT (SEQ ID NO: 552) |
| | | | | | AAATCAGCAACCTGCATCTTTTATG (SEQ ID NO: 553) |
| | | | | | GGAATTGCCTCTGGACTATCATCAA (SEQ ID NO: 554) |
| | | | | | GACCAAGACTCCCCTAGAGAACAGA (SEQ ID NO: 555) |
| | | | | | AAGCATATCAAACTACCCTCATCTT (SEQ ID NO: 556) |
| | | | | | GAGCACTGAATTCCAAGCTCCACTG (SEQ ID NO: 557) |
| | | | | | GCTCCACTGGCATTTCAGTCTGACG (SEQ ID NO: 558) |
| | | | | | GTACCTGAGACATAGACGACTTTTC (SEQ ID NO: 559) |
| 203418_at | CCNA2 | CCN1, CCNA | cyclin A2 | 890 | TACCCAATACTTTCTGCATCAGCAG (SEQ ID NO: 560) |
| | | | | | TCTGCATCAGCAGCCTGCAAACTGC (SEQ ID NO: 561) |
| | | | | | TGATAGATGCTGACCCATACCTCAA (SEQ ID NO: 562) |
| | | | | | CCATCAGTTATTGCTGGAGCTGCCT (SEQ ID NO: 563) |
| | | | | | TGGAGCTGCCTTTCATTTAGCACTC (SEQ ID NO: 564) |
| | | | | | TTTAGCACTCTACACAGTCACGGGA (SEQ ID NO: 565) |
| | | | | | GTCACGGGACAAAGCTGGCCTGAAT (SEQ ID NO: 566) |
| | | | | | GGAAAGTCTTAAGCCTTGTCTCATG (SEQ ID NO: 567) |
| | | | | | TACCTCAAAGCACCACAGCATGCAC (SEQ ID NO: 568) |
| | | | | | AAAGTATCATGGTGTTTCTCTCCTC (SEQ ID NO: 569) |
| | | | | | AAAGACTGCCTTTGTTTTCTAAGAT (SEQ ID NO: 570) |
| 201947_s_at | CCT2 | 99D8.1, CCT- | chaperonin | 10576 | GAAGCTGTTGCAATGGAGTCTTATG (SEQ ID NO: 571) |
| | | beta, CCTB, | containing TCP1, | | ATGTTGCCAACCATCATAGCTGACA (SEQ ID NO: 572) |
| | | MGC142074, | subunit 2 (beta) | | GCTATGACAGTGCAGACCTGGTGGC (SEQ ID NO: 573) |
| | | MGC142076, | | | GCTCAGGGCTGCTCACAGTGAAGGC (SEQ ID NO: 574) |
| | | PRO1633, TCP- | | | GAAGGCAATACCACTGCTGGATTGG (SEQ ID NO: 575) |
| | | 1-beta | | | AGATATGGCTATCCTGGGTATAACA (SEQ ID NO: 576) |
| | | | | | AGGTGATTCTGCGTGTGGACAACAT (SEQ ID NO: 577) |
| | | | | | GGACAACATCATCAAAGCGGCACCC (SEQ ID NO: 578) |
| | | | | | ACGTGCTGTCGATCTTTGGACCAGT (SEQ ID NO: 579) |
| | | | | | GACCAGTTTCTAGCAAAGTTGTGTT (SEQ ID NO: 580) |
| | | | | | ATTTAGCTGACCTTCGCTTTAACAT (SEQ ID NO: 581) |
| 214464_at | CDC42BPA | MRCK; MRCKA; | CDC42 binding | 8476 | ATTCGGAACTGTGAAGCACCTTATA (SEQ ID NO: 582) |
| | | PK428; | protein kinase alpha | | GTTAGTAGCCCAACAGATACATCGA (SEQ ID NO: 583) |
| | | FLJ23347; | (DMPK-like) | | GCCACCATCTGCCATTTGTTGGTTT (SEQ ID NO: 584) |
| | | KIAA0451; | | | GTAGCTGTGTACTTTCTGATCGGAG (SEQ ID NO: 585) |
| | | DKFZp686L1738; | | | GTACTTTCTGATCGGAGCTGTTTAA (SEQ ID NO: 586) |
| | | DKFZp686P1738 | | | TGTTTAAGAGTTACGGCTGGTCCCA (SEQ ID NO: 587) |
| | | | | | TCCCACCTCACTGGATCTTGATGTT (SEQ ID NO: 588) |
| | | | | | GAGGACTCTAGACAACAACTTAGCA (SEQ ID NO: 589) |
| | | | | | AACACAGACTGTCCAAGCTCTGCAG (SEQ ID NO: 590) |
| | | | | | AGCTCTGCAGTATTCAACTGTTGAT (SEQ ID NO: 591) |
| | | | | | GATGGTCCACTAACAGCAAGCAAAG (SEQ ID NO: 592) |
| 221520_s_at | CDCA8 | BOR, | cell division cycle | 55143 | TGGGACTTAGGGATCTTTCACCTGC (SEQ ID NO: 593) |
| | | BOREALIN, | associated 8 | | CCTGCTGTGCCCAGTAGTTCTGAAG (SEQ ID NO: 594) |
| | | FLJ10468, | | | GTTTATCCCTGTTTACTGAAGACCA (SEQ ID NO: 595) |
| | | FLJ12042 | | | GAGACAACTTCCATGTCTTGCTCTT (SEQ ID NO: 596) |
| | | | | | TCTTCTACCTCCCTAGTTAGTGGAA (SEQ ID NO: 597) |
| | | | | | AGGGAACTGTAGGGCCCAGATTCTG (SEQ ID NO: 598) |
| | | | | | AACTGTCTCTAAGCTATCCATGGTC (SEQ ID NO: 599) |
| | | | | | AGCAGTGAGGTCCTCATTCTCCAGC (SEQ ID NO: 600) |
| | | | | | CTGCCCTGGAGAATCATGTGCTATG (SEQ ID NO: 601) |
| | | | | | TTGAGAACTAGAGTCCTCATCCCCA (SEQ ID NO: 602) |
| | | | | | TTCTCATGTAGGGCTCTCTGTGGTA (SEQ ID NO: 603) |
| 213230_at | CDR2L | HUMPPA | cerebellar | 30850 | GCTGCGTCAGGAGTTTTGCTTCAAG (SEQ ID NO: 604) |
| | | | degeneration-related | | AGTTCAGCTAAGAGACACCCAGGTC (SEQ ID NO: 605) |
| | | | protein 2-like | | GCCCTTCCTGAGACCTTAGAAACCA (SEQ ID NO: 606) |
| | | | | | GAAACCAGAAGAGCCATACAGTCAG (SEQ ID NO: 607) |
| | | | | | TACAGTCAGTGGAAGGCGGGGGGGC (SEQ ID NO: 608) |
| | | | | | GGATCCCAGTGGGAACCTTCATGCC (SEQ ID NO: 609) |
| | | | | | ATGGGAGCTGGTGGCGACTGAGTCC (SEQ ID NO: 610) |
| | | | | | AGTCCTTCTGTACGTGCAACTGGGA (SEQ ID NO: 611) |
| | | | | | TTGTCCTTTGAGGCTAGGCAGCTCC (SEQ ID NO: 612) |
| | | | | | TCCGTGTGTGTCTGTTATCTGGGGG (SEQ ID NO: 613) |
| | | | | | CAGACCAGCGTCACCTTTGAAGTAT (SEQ ID NO: 614) |
| 207719_x_at | CEP170 | FAM68A, KAB, | centrosomal protein | 9859 | GTAACTCTATACACTTCAAGACTTT (SEQ ID NO: 615) |
| | | KIAA0470 | 170 kDa | | TAATGGATCTCCTATTTTTCTTTAA (SEQ ID NO: 616) |
| | | | | | AATGAACTATTCTCCTTGTAAAGCT (SEQ ID NO: 617) |
| | | | | | TAAAGCTAAATTCCCCATTCTGTCT (SEQ ID NO: 618) |
| | | | | | AATAATCAGCATTGCTCCCTCTTTT (SEQ ID NO: 619) |
| | | | | | ATTGCTCCCTCTTTTATTTAATATT (SEQ ID NO: 620) |
| | | | | | GGGTACTGATTATATCCACATGGAA (SEQ ID NO: 621) |
| | | | | | AATCTACTCTGCTTACATTCTTGTT (SEQ ID NO: 622) |
| | | | | | GTGTTTACACAGTTTGGTTCAATTA (SEQ ID NO: 623) |
| | | | | | CATTTGGCCTTTTACTATGTTATTT (SEQ ID NO: 624) |
| | | | | | ACATTATCCTCCTTATTTATTTTTG (SEQ ID NO: 625) |
| 207209_at | CETN1 | CEN1, CETN | centrin, EF-hand | 1068 | CTAAAGTGACTCTCTGGGTTGCCTG (SEQ ID NO: 626) |
| | | | protein, 1 | | TAGAGGACAGCGGCTGCCTGTCCCT (SEQ ID NO: 627) |
| | | | | | CTCACCCCCATAATTTGTCTAGATC (SEQ ID NO: 628) |
| | | | | | GGCCCTGAAGTTTGAGTGCGCCCTC (SEQ ID NO: 629) |
| | | | | | GGACAGCTTCTGGGACACACAAAAA (SEQ ID NO: 630) |
| | | | | | TCCCTTTGTCACTTCTTTGGTGGTC (SEQ ID NO: 631) |
| | | | | | TCTTGCTTCATATATCATTCCTTAA (SEQ ID NO: 632) |
| | | | | | AAATTCCAGTCATTGTTCCAGCATA (SEQ ID NO: 633) |
| | | | | | GCCAGTAGATTTGCCTAGCCTGTCC (SEQ ID NO: 634) |
| | | | | | TAGCCTGTCCACTTAGCTGAATACC (SEQ ID NO: 635) |
| | | | | | GTGGCCACTTACAAACTTACGGAGC (SEQ ID NO: 636) |
| 202357_s_at | CFB | DADB-122G4.5, | complement factor B | 629 | CAGACTATCAGGCCCATTTGTCTCC (SEQ ID NO: 637) |
| | | BF, BFD, CFAB, | | | TCCCCTGCACCGAGGGAACAACTCG (SEQ ID NO: 638) |
| | | FB, FBI12, GBG, | | | GAACAACTCGAGCTTTGAGGCTTCC (SEQ ID NO: 639) |
| | | H2-Bf, PBF2 | | | CAACTACCACTTGCCAGCAACAAAA (SEQ ID NO: 640) |
| | | | | | CCCTGCACAGGATATCAAAGCTCTG (SEQ ID NO: 641) |
| | | | | | GCTCTGTTTGTGTCTGAGGAGGAGA (SEQ ID NO: 642) |
| | | | | | ACTCGGAAGGAGGTCTACATCAAGA (SEQ ID NO: 643) |
| | | | | | GAAGTCGTTTCATTCAAGTTGGTGT (SEQ ID NO: 644) |
| | | | | | AAGTTGGTGTAATCAGCTGGGGAGT (SEQ ID NO: 645) |
| | | | | | GCTGGGGAGTAGTGGATGTCTGCAA (SEQ ID NO: 646) |
| | | | | | ACTTTCACATCAACCTCTTTCAAGT (SEQ ID NO: 647) |
| 218927_s_at | CHST12 | C4S-2, C4ST-2, | carbohydrate | 55501 | GACCCGCACACGGAGAAGCTGGCGC (SEQ ID NO: 648) |
| | | C4ST2 | (chondroitin 4) | | CACTGGCGGCAGGTGTACCGCCTCT (SEQ ID NO: 649) |
| | | | sulfotransferase 12 | | GCCAGATCGACTACGACTTCGTGGG (SEQ ID NO: 650) |
| | | | | | GCTGGAGACTCTGGACGAGGACGCC (SEQ ID NO: 651) |
| | | | | | GGAGGAGGACTGGTTCGCCAAGATC (SEQ ID NO: 652) |
| | | | | | TAAACTCTACGAGGCCGACTTTGTT (SEQ ID NO: 653) |
| | | | | | GAAAACCTCCTCCGAGACTGAAAGC (SEQ ID NO: 654) |
| | | | | | AAAGCTTTCGCGTTGCTTTTTCTCG (SEQ ID NO: 655) |
| | | | | | GCGTGCCTGGAACCTGACGCACGCG (SEQ ID NO: 656) |
| | | | | | TTTGCAATCTGGGCTTCTTGTTCAC (SEQ ID NO: 657) |
| | | | | | TCCACTGCCTCTATCCATTGAGTAC (SEQ ID NO: 658) |
| 209666_s_at | CHUK | IKBKA, IKK- | conserved helix-loop- | 1147 | CAATCTTTCATGTCTTGTCATACAA (SEQ ID NO: 659) |
| | | alpha, IKK1, | helix ubiquitous | | ACAAATTTATAAAGGTCTGCACTCC (SEQ ID NO: 660) |
| | | IKKA, NFKBIKA, | kinase | | TCTGCACTCCTTTATCTGTAATTGT (SEQ ID NO: 661) |
| | | TCF16 | | | AAGGTTTCCATAAGCTTGGTGACAA (SEQ ID NO: 662) |
| | | | | | ATTTGCTTGCAATCTAATCTGAACT (SEQ ID NO: 663) |
| | | | | | AATCTGAACTGACCTTGAATCTTTT (SEQ ID NO: 664) |
| | | | | | TATCCCATTTAGTGTGAATATTCCT (SEQ ID NO: 665) |
| | | | | | TGTGAATATTCCTTTATTTTGCTGC (SEQ ID NO: 666) |
| | | | | | CCACAGACTGTGGTGAGGGCTGGTT (SEQ ID NO: 667) |
| | | | | | GAGGGCTGGTTAATGTAGTATGGTA (SEQ ID NO: 668) |
| | | | | | ATATGCACAAAACTACTTTTCTAAA (SEQ ID NO: 669) |
| 214252_s_at | CLN5 | FLJ90628, NCL | ceroid-lipofuscinosis, | 1203 | TATGCATGATGCCATTGGATTCAGA (SEQ ID NO: 670) |
| | | | neuronal 5 | | GGATTCAGAAGTACATTAACTGGCA (SEQ ID NO: 671) |
| | | | | | TAACTGGCAAGAACTACACAATGGA (SEQ ID NO: 672) |
| | | | | | TGGTATGAACTTTTCCAACTTGGCA (SEQ ID NO: 673) |
| | | | | | ACTTGGCAACTGTACATTTCCCCAT (SEQ ID NO: 674) |
| | | | | | ATCTCCGACCTGAAATGGATGCCCC (SEQ ID NO: 675) |
| | | | | | ACCTGAAATGGATGCCCCTTTCTGG (SEQ ID NO: 676) |
| | | | | | ATGCCCCTTTCTGGTGTAATCAAGG (SEQ ID NO: 677) |
| | | | | | TTCTGGTGTAATCAAGGCGCTGCCT (SEQ ID NO: 678) |
| | | | | | TGCTTTTTTGAGGGAATTGATGATG (SEQ ID NO: 679) |
| | | | | | AATGGGACATTAGTTCAAGTAGCAA (SEQ ID NO: 680) |
| 204050_s_at | CLTA | CLTA2, | clathrin, light chain | 1211 | GGAACGCTTGGAAGCCCTTGATGCC (SEQ ID NO: 681) |
| | | MGC140767 | (Lca) | | ACGCTTGGAAGCCCTTGATGCCAAT (SEQ ID NO: 682) |
| | | | | | TTGGAAGCCCTTGATGCCAATTCTC (SEQ ID NO: 683) |
| | | | | | CCCTTGATGCCAATTCTCGGAAGCA (SEQ ID NO: 684) |
| | | | | | GCCAATTCTCGGAAGCAAGAAGCAG (SEQ ID NO: 685) |
| | | | | | GAATGGTATGCAAGACAGGACGAGC (SEQ ID NO: 686) |
| | | | | | GCAAGACAGGACGAGCAGCTACAGA (SEQ ID NO: 687) |
| | | | | | GACAGGACGAGCAGCTACAGAAAAC (SEQ ID NO: 688) |
| | | | | | GGAAGCTTCCCAAGAGTAGCCTCAA (SEQ ID NO: 689) |
| | | | | | CTTCCCAAGAGTAGCCTCAACCTGT (SEQ ID NO: 690) |
| | | | | | CAAGAGTAGCCTCAACCTGTGCTTC (SEQ ID NO: 691) |
| 217870_s_at | CMPK1 | CMK; UMK; | cytidine | 51727 | GGAATGAGTTCTTATCTAGTGTTGC (SEQ ID NO: 692) |
| | | CMPK; UMPK; | monophosphate | | TCTTATCTAGTGTTGCAGGCCAGCA (SEQ ID NO: 693) |
| | | UMP-CMPK1; | (UMP-CMP) kinase | | GGTTTCGAGAGCATTCCTACTCACA (SEQ ID NO: 694) |
| | | RP11-511I2.1 | 1, cytosolic | | GAGCATTCCTACTCACATAAGTGAA (SEQ ID NO: 695) |
| | | | | | TGAGACTAGGTGCTTTGCTTCCTTT (SEQ ID NO: 696) |
| | | | | | GCTTCCTTTCATCAGGTATCTTTCT (SEQ ID NO: 697) |
| | | | | | AGGTATCTTTCTGTGGCATTTGAGA (SEQ ID NO: 698) |
| | | | | | TTACTAAATTATGAGGCTTTGCTTT (SEQ ID NO: 699) |
| | | | | | TAACTAGTTTTGTCATTCCATTTGT (SEQ ID NO: 700) |
| | | | | | GATACAGTCACCAAGAATGTTTTGA (SEQ ID NO: 701) |
| | | | | | AGACCCCAATTTAAGCCTTGCTTAT (SEQ ID NO: 702) |
| 218250_s_at | CNOT7 | CAF1, hCAF-1 | CCR4-NOT | 29883 | GGTTACGACTTTGGCTACTTAATCA (SEQ ID NO: 703) |
| | | | transcription | | TCCTAACCAACTCTAACTTGCCTGA (SEQ ID NO: 704) |
| | | | complex, subunit 7 | | AGGACCACAACATCAGGCAGGATCT (SEQ ID NO: 705) |
| | | | | | GGCAGGATCTGATTCATTGCTCACA (SEQ ID NO: 706) |
| | | | | | TGCTCACAGGAATGGCCTTTTTCAA (SEQ ID NO: 707) |
| | | | | | GTGGTCATTTGTATGGCCTTGGTTC (SEQ ID NO: 708) |
| | | | | | CCTTGGTTCTGGTTCATCCTATGTA (SEQ ID NO: 709) |
| | | | | | ATTTCGAGCTACACACATGCTTGTA (SEQ ID NO: 710) |
| | | | | | GTTTTATCTCTGGTTGAATCCCTCG (SEQ ID NO: 711) |
| | | | | | GAATCCCTCGAACAATAGACAGTAC (SEQ ID NO: 712) |
| | | | | | CATGGCCCATTTTATTGTCTGCCTT (SEQ ID NO: 713) |
| 209796_s_at | CNPY2 | MGC156825, | canopy 2 homolog | 10330 | TGGAGCGACCCCATTACGCTAAAGA (SEQ ID NO: 714) |
| | | Tmem4 | (zebrafish) | | GAGCGACCCCATTACGCTAAAGATG (SEQ ID NO: 715) |
| | | | | | GACCCCATTACGCTAAAGATGAAAG (SEQ ID NO: 716) |
| | | | | | GAGCCAGGATCTCCACTGTGGAGCA (SEQ ID NO: 717) |
| | | | | | GAATGGGAAATTGCCCAGGTGGACC (SEQ ID NO: 718) |
| | | | | | ACCCCAAGAAGACCATTCAGATGGG (SEQ ID NO: 719) |
| | | | | | GACCATTCAGATGGGATCTTTCCGG (SEQ ID NO: 720) |
| | | | | | GATGGGATCTTTCCGGATCAATCCA (SEQ ID NO: 721) |
| | | | | | GGATCTTTCCGGATCAATCCAGATG (SEQ ID NO: 722) |
| | | | | | TTTCCGGATCAATCCAGATGGCAGC (SEQ ID NO: 723) |
| | | | | | TCCAGATGGCAGCCAGTCAGTGGTG (SEQ ID NO: 724) |
| 213050_at | COBL | DKFZp686G13227, | cordon-bleu homolog | 23242 | GCCATTCCCCAGGCTATGAAGTGAC (SEQ ID NO: 725) |
| | | KIAA0633, | (mouse) | | GCCTGTGTACGGAGCCTGATGACTT (SEQ ID NO: 726) |
| | | MGC131893 | | | TCACTGGGAGCCTTCTGGAATCCTG (SEQ ID NO: 727) |
| | | | | | CAGGGATGTTGGATGCCCACCTGTC (SEQ ID NO: 728) |
| | | | | | AATTTTGGTTCAGCCTTGACGCGTT (SEQ ID NO: 729) |
| | | | | | TTGACGCGTTTGTCCAACAGCTCAT (SEQ ID NO: 730) |
| | | | | | TTGGCTCCCTTTGTATGATATCGTG (SEQ ID NO: 731) |
| | | | | | AATGTTTCACAGCGATCTCAGATCT (SEQ ID NO: 732) |
| | | | | | TGGAAGCTGTTCTGCATTCACTGAT (SEQ ID NO: 733) |
| | | | | | GAGCTACCAGCCAATTGCTCTTCAT (SEQ ID NO: 734) |
| | | | | | AGCTATCTGTCTTTTAGTGCCACAA (SEQ ID NO: 735) |
| 206441_s_at | COMMD4 | FLJ20452 | COMM domain | 54939 | GGAGCTGCTGGGACAGGGGATTGAT (SEQ ID NO: 736) |
| | | | containing 4 | | AGCTCACGGCTGACGCCAAGTTTGA (SEQ ID NO: 737) |
| | | | | | TCCAGTGCGGCCAAGCACAGTGTCG (SEQ ID NO: 738) |
| | | | | | AGTGTCGATGGCGAATCCTTGTCCA (SEQ ID NO: 739) |
| | | | | | TCCTTGTCCAGTGAACTGCAGCAGC (SEQ ID NO: 740) |
| | | | | | AGCTGGGGCTGCCCAAAGAGCACGC (SEQ ID NO: 741) |
| | | | | | CCAGCCTGTGCCGCTGTTATGAGGA (SEQ ID NO: 742) |
| | | | | | CAAAGCCCCTTGCAGAAGCACTTGC (SEQ ID NO: 743) |
| | | | | | TGAACTGCTCTTCGGGAGGCAGCCC (SEQ ID NO: 744) |
| | | | | | GCCCTGGTTCTAGGATGCTGAGGCC (SEQ ID NO: 745) |
| | | | | | GCCTCACTTCTCTCTTGAGAACTTG (SEQ ID NO: 746) |
| 202078_at | COPS3 | SGN3 | COP9 constitutive | 8533 | GACCTTTTTAACTCTATCATTACAA (SEQ ID NO: 747) |
| | | | photomorphogenic | | ATGGCAAGTCGTGTGCAGTTGTCTG (SEQ ID NO: 748) |
| | | | homolog subunit 3 | | TGTGCAGTTGTCTGGACCTCAGGAG (SEQ ID NO: 749) |
| | | | (Arabidopsis) | | TGGTCAGTTTCCATGATAACCCTGA (SEQ ID NO: 750) |
| | | | | | ATAACCCAGCCATGCTTCATAACAT (SEQ ID NO: 751) |
| | | | | | GGAGATCACAGTGAACCCTCAGTTT (SEQ ID NO: 752) |
| | | | | | GAAACAAACCATCCAGTTATTCTTG (SEQ ID NO: 753) |
| | | | | | AAGAGAAACTACCATCTTGGCCAGT (SEQ ID NO: 754) |
| | | | | | GTGACAAGTGTTCGGAGGGCAGCAG (SEQ ID NO: 755) |
| | | | | | CAGAGAGGACCAAGCCTGTGTCACC (SEQ ID NO: 756) |
| | | | | | TTGACATCTTCAGTCCTGTGTGCTT (SEQ ID NO: 757) |
| 218328_at | COQ4 | OSTLU_119511 | coenzyme Q4 | 51117 | AAGTGCTGGTCTCGGAGTTGATCCC (SEQ ID NO: 758) |
| | | | homolog (S. cerevisiae) | | GTTGATCCCATGGGCCGTTCAGAAC (SEQ ID NO: 759) |
| | | | | | AACGGGCGCAGAGCCCATTGTGTCC (SEQ ID NO: 760) |
| | | | | | CAACCTGTACTATGAGCGGCGCTGG (SEQ ID NO: 761) |
| | | | | | AGGAGCTGGGCATTACAGCACCACC (SEQ ID NO: 762) |
| | | | | | TCTTCTTTGAACACTGACCCTTGGA (SEQ ID NO: 763) |
| | | | | | CTGAGTGGCCTTGAGGACGAACCCC (SEQ ID NO: 764) |
| | | | | | GCAAGCAGTACAGTGGCATTCCCAG (SEQ ID NO: 765) |
| | | | | | GAACAGTATCAGTCGTCTGGGCTCA (SEQ ID NO: 766) |
| | | | | | ATTGTGCAGTGCTCCTGTTGCAACT (SEQ ID NO: 767) |
| | | | | | GGAGTGAGGGCTACACCCAATTCCA (SEQ ID NO: 768) |
| 203663_s_at | COX5A | COX, COX-VA, | cytochrome c oxidase | 9377 | GTTTGATGCTCGCTGGGTAACATAC (SEQ ID NO: 769) |
| | | VA | subunit Va | | TGCTCGCTGGGTAACATACTTCAAC (SEQ ID NO: 770) |
| | | | | | AACACACTTGTTACCTATGATATGG (SEQ ID NO: 771) |
| | | | | | CCTATGATATGGTTCCAGAGCCCAA (SEQ ID NO: 772) |
| | | | | | CGGAGGAACTGGGCCTTGACAAAGT (SEQ ID NO: 773) |
| | | | | | GGGCCTTGACAAAGTGTAAACCGCA (SEQ ID NO: 774) |
| | | | | | GTAAACCGCATGGATGGGCTTCCCC (SEQ ID NO: 775) |
| | | | | | GGCTTCCCCAAGGATTTATTGACAT (SEQ ID NO: 776) |
| | | | | | TTGCTACTTGAGTGTGAACAGTTAC (SEQ ID NO: 777) |
| | | | | | GAACAGTTACCTGGAAATACTGATG (SEQ ID NO: 778) |
| | | | | | CCTTTATTGAGTACCAAGCCATGTA (SEQ ID NO: 779) |
| 218871_x_at | CSGALNACT2 | CSGalNAcT-2, | chondroitin sulfate N- | 55454 | AAAATCATTACTCTTTTTCTCAGTA (SEQ ID NO: 780) |
| | | DKFZp686H13226, | acetylgalactosaminyltransferase 2 | | AATATTACAATAACTCTTACCTAAT (SEQ ID NO: 781) |
| | | FLJ43310, | | | TAACTCTTACCTAATTATTCTTACA (SEQ ID NO: 782) |
| | | GALNACT-2, | | | AGATGTGTGAAATGTTCTCTGCAAA (SEQ ID NO: 783) |
| | | GALNACT2, | | | AATAATTCAGGCCACTGTCTCCTTT (SEQ ID NO: 784) |
| | | MGC40204, | | | GCCACTGTCTCCTTTTATATATTAT (SEQ ID NO: 785) |
| | | PRO0082 | | | GAAGACCAGTGAATTACGATATTTA (SEQ ID NO: 786) |
| | | | | | GGTAAGTTACAGCTTGTTTTTTGAG (SEQ ID NO: 787) |
| | | | | | ATGAGTTTACTTTTGTTCCTGTTGT (SEQ ID NO: 788) |
| | | | | | TGTTCCTGTTGTTTTTAACTAGCTT (SEQ ID NO: 789) |
| | | | | | ATGGAAATGCTATACGTTTTTGACA (SEQ ID NO: 790) |
| 222235_s_at | CSGALNACT2 | CSGalNAcT-2, | chondroitin sulfate N- | 55454 | ATATTCAGATAGGACTGCACTACAT (SEQ ID NO: 791) |
| | | DKFZp686H13226, | acetylgalactosaminyltransferase 2 | | CAGATAGGACTGCACTACATTATTT (SEQ ID NO: 792) |
| | | FLJ43310, | | | ATCTGTTACCATCAGGTCAATTCCT (SEQ ID NO: 793) |
| | | GALNACT-2, | | | GTTACCATCAGGTCAATTCCTAGTA (SEQ ID NO: 794) |
| | | GALNACT2, | | | AGGTCAATTCCTAGTATGCATAAAT (SEQ ID NO: 795) |
| | | MGC40204, | | | GAGACCTATGTTGAAAACCCCTGAA (SEQ ID NO: 796) |
| | | PRO0082 | | | TTTTTCTCAGTAAATCATATCATCT (SEQ ID NO: 797) |
| | | | | | ATTTCTAGGTGCTATATTAATTCAA (SEQ ID NO: 798) |
| | | | | | TGTGAAATGTTCTCTGCAAAATAAT (SEQ ID NO: 799) |
| | | | | | GCAAAATAATTCAGGCCACTGTCTC (SEQ ID NO: 800) |
| | | | | | TTTGCAAAGGTAAGTTACAGCTTGT (SEQ ID NO: 801) |
| 202156_s_at | CUGBP2 | BRUNOL3, ETR- | CUG triplet repeat, | 10659 | ATTTCTAAGACCATTTCATTCTGAA (SEQ ID NO: 802) |
| | | 3, NAPOR | RNA binding protein 2 | | TCTGAAACTTCTTATCAATTACCTA (SEQ ID NO: 803) |
| | | | | | TTTCTTCTTCACAAGTCACCAAGAG (SEQ ID NO: 804) |
| | | | | | TTCTCCAAAAAATAACCTTCCACAG (SEQ ID NO: 805) |
| | | | | | AGAGACAAACTGTCCTTCTATCCAC (SEQ ID NO: 806) |
| | | | | | CTTCTATCCACTTTTATCTTTTAAT (SEQ ID NO: 807) |
| | | | | | GCAAGGGTGCAAAGGGCCTGTGCCA (SEQ ID NO: 808) |
| | | | | | GATACAATGGCAGTCCTCAAAGGCG (SEQ ID NO: 809) |
| | | | | | AAGGCGTAACGAGTTCATCTTTCTT (SEQ ID NO: 810) |
| | | | | | TCATCTTTCTTTCACCATAGGGGTT (SEQ ID NO: 811) |
| | | | | | ATAGTTGGCTTGTGCTACTCTGGAA (SEQ ID NO: 812) |
| 204533_at | CXCL10 | C7, IFI10, INP10, | chemokine (C—X—C | 3627 | TAACTCTACCCTGGCACTATAATGT (SEQ ID NO: 813) |
| | | IP-10, SCYB10, | motif) ligand 10 | | GCTCTACTGAGGTGCTATGTTCTTA (SEQ ID NO: 814) |
| | | crg-2, gIP-10, | | | TCTTAGTGGATGTTCTGACCCTGCT (SEQ ID NO: 815) |
| | | mob-1 | | | TCTGACCCTGCTTCAAATATTTCCC (SEQ ID NO: 816) |
| | | | | | GGTACTAAGGAATCTTTCTGCTTTG (SEQ ID NO: 817) |
| | | | | | GCTTTGGGGTTTATCAGAATTCTCA (SEQ ID NO: 818) |
| | | | | | AATGCTCTTTACTTCATGGACTTCC (SEQ ID NO: 819) |
| | | | | | TTACTTCATGGACTTCCACTGCCAT (SEQ ID NO: 820) |
| | | | | | ATTCTTTCAGTGGCTACCTACATAC (SEQ ID NO: 821) |
| | | | | | GCTACCTACATACAATTCCAAACAC (SEQ ID NO: 822) |
| | | | | | AATGAGGTACTCTCCTGGAAATATT (SEQ ID NO: 823) |
| 210163_at | CXCL11 | H174, I-TAC, IP- | chemokine (C—X—C | 6373 | TTCTTTCCCCAAATATCATGTAGCA (SEQ ID NO: 824) |
| | | 9, IP9, | motif) ligand 11 | | GAAACATTCTTATGCATCATTTGGT (SEQ ID NO: 825) |
| | | MGC102770, | | | TACGCTATGGGATACTGGCAACAGT (SEQ ID NO: 826) |
| | | SCYB11, | | | GCAACAGTGCACATATTTCATAACC (SEQ ID NO: 827) |
| | | SCYB9B, b-R1 | | | TTCATAACCAAATTAGCAGCACCGG (SEQ ID NO: 828) |
| | | | | | GCAGCACCGGTCTTAATTTGATGTT (SEQ ID NO: 829) |
| | | | | | GGATATGTGTGTTTACTGTACTTTT (SEQ ID NO: 830) |
| | | | | | TTTGTTTTGATCCGTTTGTATAAAT (SEQ ID NO: 831) |
| | | | | | GATAGCAATATCTTGGACACATTTG (SEQ ID NO: 832) |
| | | | | | ATACCTAGCAATCACTTTTACTTTT (SEQ ID NO: 833) |
| | | | | | TTTTACTTTTTGTAATTCTGTCTCT (SEQ ID NO: 834) |
| 211122_s_at | CXCL11 | H174, I-TAC, IP- | chemokine (C—X—C | 6373 | GAGACTTTTCTATGGTTTTGTGACT (SEQ ID NO: 835) |
| | | 9, IP9, | motif) ligand 11 | | GTGACTTTCAACTTTTGTACAGTTA (SEQ ID NO: 836) |
| | | MGC102770, | | | GACAATCAGAATTCCACTGCCCAAA (SEQ ID NO: 837) |
| | | SCYB11, | | | GGCTGGTTACCATCGGAGTTTACAA (SEQ ID NO: 838) |
| | | SCYB9B, b-R1 | | | TGCTTTCACGTTCTTACTTGTTGTA (SEQ ID NO: 839) |
| | | | | | TACATTCATGCATTTCTAGGCTAGA (SEQ ID NO: 840) |
| | | | | | AGAGAACCTTCTAGATTTGATGCTT (SEQ ID NO: 841) |
| | | | | | GTCTCTAGAAGTTATCTGTCTGTAT (SEQ ID NO: 842) |
| | | | | | CTGTCTGTATTGATCTTTATGCTAT (SEQ ID NO: 843) |
| | | | | | GCTATATTACTATCTGTGGTTACAG (SEQ ID NO: 844) |
| | | | | | TACTGGAGTCAAGCCCTTATAAGTC (SEQ ID NO: 845) |
| 209774_x_at | CXCL2 | CINC-2a, GROb, | chemokine (C—X—C | 2920 | AGAGAGACACAGCTGCAGAGGCCAC (SEQ ID NO: 846) |
| | | Gro2, MIP-2, | motif) ligand 2 | | ACCTGGATTGCGCCTAATGTGTTTG (SEQ ID NO: 847) |
| | | MIP-2a, Mgsa-b, | | | TGATTGAATCTACTTGCACACTCTC (SEQ ID NO: 848) |
| | | Mip2, Scyb, | | | GCACACTCTCCCATTATATTTATTG (SEQ ID NO: 849) |
| | | Scyb2 | | | AACCCAAGTTAGTTCAATCCTGATT (SEQ ID NO: 850) |
| | | | | | GAAGGTTTGCAGATATTCTCTAGTC (SEQ ID NO: 851) |
| | | | | | GACATATCACATGTCAGCCACTGTG (SEQ ID NO: 852) |
| | | | | | ATGGCCAGTAAGATCAATGTGACGG (SEQ ID NO: 853) |
| | | | | | TGTGACGGCAGGGAAATGTATGTGT (SEQ ID NO: 854) |
| | | | | | CAGTGTGTGGTCAACATTTCTCATG (SEQ ID NO: 855) |
| | | | | | ATGTTAATTATGCAGTGTTTCCCTC (SEQ ID NO: 856) |
| 217200_x_at | CYB561 | FRRS2 | cytochrome b-561 | 1534 | CTGCTGCCTCTGTTCTGGAGGACGA (SEQ ID NO: 857) |
| | | | | | TTCTGGAGGACGAGCCTTCTCCTTA (SEQ ID NO: 858) |
| | | | | | GCTGCCCATCTTTCCAGGAAGTCAG (SEQ ID NO: 859) |
| | | | | | AGTCAGACAACTAACGATCATCCCC (SEQ ID NO: 860) |
| | | | | | ACATCACTCCAGCCCCATAAAGAGT (SEQ ID NO: 861) |
| | | | | | GTGTCATGTTAGCTGAGTCACCATT (SEQ ID NO: 862) |
| | | | | | TCACCATTTGGCTTCGGCCTGGAAA (SEQ ID NO: 863) |
| | | | | | ACACTGATCGTGTGCGAGGCCAGGA (SEQ ID NO: 864) |
| | | | | | AAGACCATCCTGACTAACAAACACA (SEQ ID NO: 865) |
| | | | | | GTCTCTACGGAATTCCCTGTATTAG (SEQ ID NO: 866) |
| | | | | | GGATGCCTTTTCTGTGATTGGGTGG (SEQ ID NO: 867) |
| 201634_s_at | CYB5B | CYB5-M, | cytochrome b5 type B | 80777 | GTGGTGCCGGACAAAAAACTACATG (SEQ ID NO: 868) |
| | | CYPB5M, | (outer mitochondrial | | CAAAAAACTACATGGCCCTTTCGTG (SEQ ID NO: 869) |
| | | DKFZp686M0619, | membrane) | | GGGAGGGATGAATTGGGGTGATAGA (SEQ ID NO: 870) |
| | | OMB5 | | | GGTGATAGAACCCTGGTGAATTCAG (SEQ ID NO: 871) |
| | | | | | GTGAATTCAGAGTAATCTTTCTTTA (SEQ ID NO: 872) |
| | | | | | GATAGGAGTTTAGAGAAGGCACCAA (SEQ ID NO: 873) |
| | | | | | GAAGGCACCAAAGCTTTCACTTTGG (SEQ ID NO: 874) |
| | | | | | CAAAGCTTTCACTTTGGTTTGGCAC (SEQ ID NO: 875) |
| | | | | | CCTAGCTATCTTTTATTGGTAAAAT (SEQ ID NO: 876) |
| | | | | | TGTATAATTATGTTTGTAGAGCTTT (SEQ ID NO: 877) |
| | | | | | GTTTGTAGAGCTTTACCAAGGAGTT (SEQ ID NO: 878) |
| 200794_x_at | DAZAP2 | KIAA0058, | DAZ associated | 9802 | ATAGGTTGTCTCTGCATACACGAAC (SEQ ID NO: 879) |
| | | MGC14319, | protein 2 | | TACACGAACCTAACCCAAATTTGCT (SEQ ID NO: 880) |
| | | MGC766 | | | GATGTCGTGCAAACTGTACTGTGAA (SEQ ID NO: 881) |
| | | | | | GAGGAGGATGCATTTCAAAAGCTTG (SEQ ID NO: 882) |
| | | | | | GGTTACCATTTTTTGTCAGAGTGTC (SEQ ID NO: 883) |
| | | | | | AGAGTGTCTGATGCGGCCACTCATT (SEQ ID NO: 884) |
| | | | | | GGCTCCCCAGAATTCCTAGACTGGG (SEQ ID NO: 885) |
| | | | | | AATGACTTGAGTCCAGTGAAATCTC (SEQ ID NO: 886) |
| | | | | | GTTTGACTGTACCATTGACTGTTAT (SEQ ID NO: 887) |
| | | | | | AAGTTCAGCGTTGTATGTCTCTCTC (SEQ ID NO: 888) |
| | | | | | CTCTCTCTACACTGTGGTGCACTTA (SEQ ID NO: 889) |
| 208675_s_at | DDOST | AGE-R1, | dolichyl- | 1650 | CATCGTGATCCAGCAGCTCTCAAAT (SEQ ID NO: 890) |
| | | KIAA0115, | diphosphooligosaccharide- | | CTCAAATGGCAAATGGGTCCCCTTT (SEQ ID NO: 891) |
| | | MGC2191, | protein | | GGTCCCCTTTGATGGCGATGACATT (SEQ ID NO: 892) |
| | | OK/SW-cl.45, | glycosyltransferase | | GGAGTTTGTCCGCATTGATCCTTTT (SEQ ID NO: 893) |
| | | OST, OST48, | | | GATCCTTTTGTGAGGACCTTCCTGA (SEQ ID NO: 894) |
| | | WBP1 | | | ACAGTGTTCAGTTCAAGTTGCCCGA (SEQ ID NO: 895) |
| | | | | | GTTGCCCGACGTGTATGGTGTATTC (SEQ ID NO: 896) |
| | | | | | GATTACAACCGGCTAGGCTACACAC (SEQ ID NO: 897) |
| | | | | | CCTGTACTCTTCCACTCAGGTATCC (SEQ ID NO: 898) |
| | | | | | CACACGCAGTATGAGCGCTTCATCC (SEQ ID NO: 899) |
| | | | | | ATGGCACAACTTTACCTCTGTGGGA (SEQ ID NO: 900) |
| 201440_at | DDX23 | MGC8416, | DEAD (Asp-Glu-Ala- | 9416 | AGGACGACGCTGTCGGAGGCAGGGA (SEQ ID NO: 901) |
| | | PRPF28, U5- | Asp) box polypeptide | | GAGAGCAAATTACCACAGCTTCTTG (SEQ ID NO: 902) |
| | | 100K, U5-100KD, | 23 | | TTTGGGATTGCACTGGGCCATCAGC (SEQ ID NO: 903) |
| | | prp28 | | | CCATCAGCTCATGCCAGGCTATGGG (SEQ ID NO: 904) |
| | | | | | TGGGGGCAGCCAGTTGGCATTGCTC (SEQ ID NO: 905) |
| | | | | | CATTGCTCCCCAGACTGAACAGAAA (SEQ ID NO: 906) |
| | | | | | ACTGCAGTAGCATCATTGCCCTAGA (SEQ ID NO: 907) |
| | | | | | GTCATCTGGACAGCTGTTTTCCTGT (SEQ ID NO: 908) |
| | | | | | AGAGTCTTTAGACCTGTGCACAGCC (SEQ ID NO: 909) |
| | | | | | CCAAGGGGTGCTGTATGCTCTAGGC (SEQ ID NO: 910) |
| | | | | | TGAACTGGCTGTGTCCATCTTTGTC (SEQ ID NO: 911) |
| 221885_at | DENND2A | FAM31D, | DENN/MADD domain | 27147 | TTGTGGGACACTACTCTTTGTTCCT (SEQ ID NO: 912) |
| | | KIAA1277 | containing 2A | | CCTGACGTCGGGCGAGCGTGAGGAG (SEQ ID NO: 913) |
| | | | | | GCGTGAGGAGAGAACCCTGCAGCGG (SEQ ID NO: 914) |
| | | | | | TGGAGACTCAGATGTTTCGGGGCTT (SEQ ID NO: 915) |
| | | | | | TCTGTTTGAGGTCCGAGCCCAAGAG (SEQ ID NO: 916) |
| | | | | | AACACTCCCCAGTGGAGAGCACAGC (SEQ ID NO: 917) |
| | | | | | AAGGGAAAACTTCCTCCTAGTGCAG (SEQ ID NO: 918) |
| | | | | | CCTTTCTGAAAGGCTGGGTCCCAGG (SEQ ID NO: 919) |
| | | | | | TCCCAGGTTGTCACGGTGCGGAACT (SEQ ID NO: 920) |
| | | | | | AGAAAGGGAGCCCGAGACCAGGCGT (SEQ ID NO: 921) |
| | | | | | GTGTCGCCGACATGCAAATGGGTTG (SEQ ID NO: 922) |
| 53991_at | DENND2A | FAM31D, | DENN/MADD domain | 27147 | TTTGTTCCTGACGTCGGGCGAGCGT (SEQ ID NO: 923) |
| | | KIAA1277 | containing 2A | | GAGGCCTTCCGCAAAGCTGTCTCCT (SEQ ID NO: 924) |
| | | | | | TGGAGACTCAGATGTTTCGGGGCTT (SEQ ID NO: 925) |
| | | | | | GGTCCGAGCCCAAGAGTATCTGGAA (SEQ ID NO: 926) |
| | | | | | GAGCACAGCGGTGTCAATAAGTTCC (SEQ ID NO: 927) |
| | | | | | GTCAATAAGTTCCTGAAGGGACTAG (SEQ ID NO: 928) |
| | | | | | TTCCTGAAGGGACTAGGCAATAAAA (SEQ ID NO: 929) |
| | | | | | CAAGAAATAACTCTCAGCCTCAAGG (SEQ ID NO: 930) |
| | | | | | TAACTCTCAGCCTCAAGGGAAAACT (SEQ ID NO: 931) |
| | | | | | GGAAAACTTCCTCCTAGTGCAGCCC (SEQ ID NO: 932) |
| | | | | | GTCCCAGGTTGTCACGGTGCGGAAC (SEQ ID NO: 933) |
| | | | | | TCCCAGGTTGTCACGGTGCGGAACT (SEQ ID NO: 934) |
| | | | | | TCTGGCCGAGGCTGGGCTCTTCCCT (SEQ ID NO: 935) |
| | | | | | GGGCTCTTCCCTGGATGAGGACCTG (SEQ ID NO: 936) |
| | | | | | CGTGTCGCCGACATGCAAATGGGTT (SEQ ID NO: 937) |
| | | | | | GCCGACATGCAAATGGGTTGTTTTG (SEQ ID NO: 938) |
| 221081_s_at | DENND2D | FLJ22457, RP5- | DENN/MADD domain | 79961 | TTCTCACTTTTCATCCAGGAAGCCG (SEQ ID NO: 939) |
| | | 1180E21.2 | containing 2D | | AAGAGCAAGAATCCTCCTGCAGGCT (SEQ ID NO: 940) |
| | | | | | GAGCTACACACCATTTCTGGACTTC (SEQ ID NO: 941) |
| | | | | | GGATCAGCAAAACTGTCAGCTCCCA (SEQ ID NO: 942) |
| | | | | | ATCCTCACTCTGAGTCTTGGTATCC (SEQ ID NO: 943) |
| | | | | | GGTGTCTGAGATTTGGATCCCTGGT (SEQ ID NO: 944) |
| | | | | | GACACCATGCTCACAGAACTGGGCT (SEQ ID NO: 945) |
| | | | | | GGCTCAGAGCTCCATTTTTTGCAGA (SEQ ID NO: 946) |
| | | | | | TGGTTAGAAGCTGCTGCAACTGCCC (SEQ ID NO: 947) |
| | | | | | TTTGTGGTCGAAAGGCTCAGCCTCT (SEQ ID NO: 948) |
| | | | | | GCTCAGCCTCTCTACATGAAGTCTG (SEQ ID NO: 949) |
| 218021_at | DHRS4 /// | NRDR; | dehydrogenase/reductase | 10901 | CAGGATGCTCTGGATGGACAAGGAA (SEQ ID NO: 950) |
| | DHRS4L2 | DHRS4L2; SDR- | (SDR family) | 317749 | GCGGATAAGAAGGTTAGGCGAGCCA (SEQ ID NO: 951) |
| | | SRL; humNRDR; | member 4 /// | | TTAGGCGAGCCAGAGGATTGTGCTG (SEQ ID NO: 952) |
| | | FLJ11008; | dehydrogenase/reductase | | GTCTTTCCTGTGCTCTGAAGATGCC (SEQ ID NO: 953) |
| | | SCAD-SRL /// | (SDR family) | | GAAGATGCCAGCTACATCACTGGGG (SEQ ID NO: 954) |
| | | MGC905 | member 4 like 2 | | GTCCCGCCTCTGAGGACCGGGAGAC (SEQ ID NO: 955) |
| | | | | | CCTTACTGTTCACCTCATCAAATCA (SEQ ID NO: 956) |
| | | | | | CCTCATCAAATCAGTTCTGCCCTGT (SEQ ID NO: 957) |
| | | | | | CTGCCGTCAAGGTGGCGTCTTACTC (SEQ ID NO: 958) |
| | | | | | TGTTGTGGCCTTGGGTAAAGGCCTC (SEQ ID NO: 959) |
| | | | | | CCTCCCCTGAGAACACAGGACAGGC (SEQ ID NO: 960) |
| 201385_at | DHX15 | DBP1, DDX15, | DEAH (Asp-Glu-Ala- | 1665 | AAGTTCGAGTTGTGCTCTTCACGTT (SEQ ID NO: 961) |
| | | HRH2, PRP43, | His) box polypeptide | | TGTGCTCTTCACGTTGGTTCGATAA (SEQ ID NO: 962) |
| | | PRPF43, | 15 | | CACGTTGGTTCGATAATGGCCTTTA (SEQ ID NO: 963) |
| | | PrPp43p | | | GTAAATATTCCATTCTGATTTCATA (SEQ ID NO: 964) |
| | | | | | ATTAAACATTTATGCCTCCCTTTTG (SEQ ID NO: 965) |
| | | | | | CCTCCCTTTTGTGTTGACACTGTAG (SEQ ID NO: 966) |
| | | | | | GTTGACACTGTAGCTCATACTGGAA (SEQ ID NO: 967) |
| | | | | | GTGATTATCGACCATGGTATGCATG (SEQ ID NO: 968) |
| | | | | | GGTATGCATGATCGTTGTAATTGTT (SEQ ID NO: 969) |
| | | | | | TTTTTTGTTTCAGTACCAGAGGCAC (SEQ ID NO: 970) |
| | | | | | GTACCAGAGGCACTGACTTCAATAA (SEQ ID NO: 971) |
| 201386_s_at | DHX15 | DBP1, DDX15, | DEAH (Asp-Glu-Ala- | 1665 | GGAGATCATCTGACACTGCTGAACG (SEQ ID NO: 972) |
| | | HRH2, PRP43, | His) box polypeptide | | TGCTGAACGTCTACCATGCTTTTAA (SEQ ID NO: 973) |
| | | PRPF43, | 15 | | CAACTTCATTAACTACAGGTCCCTG (SEQ ID NO: 974) |
| | | PrPp43p | | | GTACGCCAGCAGCTATCTCGAATTA (SEQ ID NO: 975) |
| | | | | | GATTTAATTTGCCTCGTCGAAGTAC (SEQ ID NO: 976) |
| | | | | | GCAGGTGGCACATTTAGAACGAACA (SEQ ID NO: 977) |
| | | | | | AACCAGGTGGTTCAGTTGCATCCCT (SEQ ID NO: 978) |
| | | | | | CCTCTACTGTTCTTGACCACAAACC (SEQ ID NO: 979) |
| | | | | | GGTTGGTGAAAATTGCCCCTCAATA (SEQ ID NO: 980) |
| | | | | | ATGACATGAGCAATTTCCCACAGTG (SEQ ID NO: 981) |
| | | | | | GAGACAGTTGGACCGCATCATTGCC (SEQ ID NO: 982) |
| 210469_at | DLG5 | RP13-39P12.4, | discs, large homolog | 9231 | AGAGGCTTCCTAAGGCTGTTTTCTT (SEQ ID NO: 983) |
| | | KIAA0583, LP- | 5 (Drosophila) | | TTTCTTAGCCGTGGAGAAGCCCGCA (SEQ ID NO: 984) |
| | | DLG, P-DLG5, | | | CTACATGCTCCCAAGTGCTGTCATG (SEQ ID NO: 985) |
| | | PDLG | | | CATGAGCACGTTCCTGACAAGTCAG (SEQ ID NO: 986) |
| | | | | | AGTCAGGTGTTCAGATTGCAGTCCC (SEQ ID NO: 987) |
| | | | | | GGCCAACGTCAGGATTCTTACAGGT (SEQ ID NO: 988) |
| | | | | | GAATGTTAAGCTCACCGATCTTGGC (SEQ ID NO: 989) |
| | | | | | TGCCTGCTCATTTTCACACGTGCAG (SEQ ID NO: 990) |
| | | | | | GCAGTGGTGGGTCTGTCTCATAGCA (SEQ ID NO: 991) |
| | | | | | GTGGGCAGCGGAGGCTAGGGGCCCA (SEQ ID NO: 992) |
| | | | | | GGCAGTCCCGTTTTGCAGCGGGATT (SEQ ID NO: 993) |
| 203301_s_at | DMTF1 | DMP1, DMTF, | cyclin D binding myb- | 9988 | GTAACAAAACCCACCAGAATCTAAG (SEQ ID NO: 994) |
| | | FLJ41265, | like transcription | | CAGAATCTAAGCAGTTTTCACCCCC (SEQ ID NO: 995) |
| | | hDMP1 | factor 1 | | CCCCCTCAGAAACCACTGTCATTAG (SEQ ID NO: 996) |
| | | | | | AGTAATGTTACCTATCCTTGATACC (SEQ ID NO: 997) |
| | | | | | CTATCCTTGATACCATGACCATTTA (SEQ ID NO: 998) |
| | | | | | GAATAGTTTGTCATCCACTTAGTGT (SEQ ID NO: 999) |
| | | | | | CTTAGTGTGTTAGCTGGTGGGGTAC (SEQ ID NO: 1000) |
| | | | | | GGGGTACAATATAACCTCTCATCTC (SEQ ID NO: 1001) |
| | | | | | CTAAGAATCATTCCTGTACTACAGA (SEQ ID NO: 1002) |
| | | | | | GATCTTCACAAAGTTGTCTTTTCAC (SEQ ID NO: 1003) |
| | | | | | CACTGTGTTTTGTCAACGTGAAATT (SEQ ID NO: 1004) |
| 222070_at | DND1 | RBMS4; | dead end homolog 1 | 373863 | TGCTGGGACAGGGACCTATGGCACC (SEQ ID NO: 1005) |
| | | MGC34750 | (zebrafish) | | GGCAGCTTAGGTTTGGGTTAAGTTG (SEQ ID NO: 1006) |
| | | | | | TAAGTTGTGGTGAGGGGCCTTGCCC (SEQ ID NO: 1007) |
| | | | | | GGGTCCAACCTGACCCAGTGATCTT (SEQ ID NO: 1008) |
| | | | | | TTCTTTATTGTCCTGATTTGTCCTC (SEQ ID NO: 1009) |
| | | | | | TGATTTGTCCTCTTTGTTGGTTTTT (SEQ ID NO: 1010) |
| | | | | | GAAGGGCAGCTGAGCCAAAGGGGTC (SEQ ID NO: 1011) |
| | | | | | GGGTCAGAAATTCTGCCCTTTGCCT (SEQ ID NO: 1012) |
| | | | | | CCTCCACCACATGGCATTCTGGTTT (SEQ ID NO: 1013) |
| | | | | | CATGGCATTCTGGTTTTGGTTTCTG (SEQ ID NO: 1014) |
| | | | | | GGTCTTTCTATGCTGGTTGTATTTA (SEQ ID NO: 1015) |
| 57739_at | DND1 | RBMS4; | dead end homolog 1 | 373863 | TCAGCCTCCCTGCTGGGACAGGGAC (SEQ ID NO: 1016) |
| | | MGC34750 | (zebrafish) | | CCCTGCTGGGACAGGGACCTATGGC (SEQ ID NO: 1017) |
| | | | | | AGCTTAGCATGAATCTTCTTTATTG (SEQ ID NO: 1018) |
| | | | | | GCTTAGCATGAATCTTCTTTATTGT (SEQ ID NO: 1019) |
| | | | | | AGCATGAATCTTCTTTATTGTCCTG (SEQ ID NO: 1020) |
| | | | | | GCATGAATCTTCTTTATTGTCCTGA (SEQ ID NO: 1021) |
| | | | | | GCAGCTGAGCCAAAGGGGTCAGAAA (SEQ ID NO: 1022) |
| | | | | | GGGTCAGAAATTCTGCCCTTTGCCT (SEQ ID NO: 1023) |
| | | | | | GGTCAGAAATTCTGCCCTTTGCCTC (SEQ ID NO: 1024) |
| | | | | | CCACCACATGGCATTCTGGTTTTGG (SEQ ID NO: 1025) |
| | | | | | CACCACATGGCATTCTGGTTTTGGT (SEQ ID NO: 1026) |
| | | | | | ACCACATGGCATTCTGGTTTTGGTT (SEQ ID NO: 1027) |
| | | | | | CCACATGGCATTCTGGTTTTGGTTT (SEQ ID NO: 1028) |
| | | | | | CACATGGCATTCTGGTTTTGGTTTC (SEQ ID NO: 1029) |
| | | | | | ACATGGCATTCTGGTTTTGGTTTCT (SEQ ID NO: 1030) |
| | | | | | CATGGCATTCTGGTTTTGGTTTCTG (SEQ ID NO: 1031) |
| 212538_at | DOCK9 | RP11-155N3.2, | dedicator of | 23348 | GTGAACCAGGAGATTTAGTGCTTTT (SEQ ID NO: 1032) |
| | | DKFZp686C11110, | cytokinesis 9 | | AAGCAGTTTAGAACCAAAGGCCTAT (SEQ ID NO: 1033) |
| | | DKFZp686D2047, | | | AAGGCCTATATTAATAACCGCAACT (SEQ ID NO: 1034) |
| | | DKFZp686N04132, | | | TAACCGCAACTATGCTGAAAAGTAC (SEQ ID NO: 1035) |
| | | FLJ11949, | | | ATCATTGTTAATACAGTCCTGGCAT (SEQ ID NO: 1036) |
| | | FLJ16744, | | | AGTCCTGGCATTCTGTACATATATG (SEQ ID NO: 1037) |
| | | FLJ44528, | | | AATACTCACATGGGCTTATGCATTA (SEQ ID NO: 1038) |
| | | FLJ45282, | | | TGTGCTGTTCCAGTATATGCAATAC (SEQ ID NO: 1039) |
| | | FLJ45601, | | | GGAGATGTATACAGTCTTTACTATA (SEQ ID NO: 1040) |
| | | KIAA1058, | | | AATCCTGTGGTGAATGGTGGTGTAC (SEQ ID NO: 1041) |
| | | KIAA1085, ZIZ1, | | | AGCTGTCACCATGTTATATTTTCTT (SEQ ID NO: 1042) |
| | | ZIZIMIN1 | | | |
| 205595_at | DSG3 | CDHF6, | desmoglein 3 | 1830 | CCATCCCATAGAAGTCCAGCAGACA (SEQ ID NO: 1043) |
| | | DKFZp686P23184, | (pemphigus vulgaris | | AGTCAAGGAGCTTCTGCTTTGTCCG (SEQ ID NO: 1044) |
| | | PVA | antigen) | | CTGACCCTCTGCAGCATGGTAACTA (SEQ ID NO: 1045) |
| | | | | | AGTAACGGAGACTTACTCGGCTTCT (SEQ ID NO: 1046) |
| | | | | | AGAAAGGGTGATCTGTCCCATTTCC (SEQ ID NO: 1047) |
| | | | | | CCATTTCCAGTGTTCCTGGCAACCT (SEQ ID NO: 1048) |
| | | | | | GCCCAACGCAGCTACGAGGGTCACA (SEQ ID NO: 1049) |
| | | | | | GAGGGTCACATACTATGCTCTGTAC (SEQ ID NO: 1050) |
| | | | | | CCTTGCTCCCGTCTAATATGACCAG (SEQ ID NO: 1051) |
| | | | | | TGAGCTGGAATACCACACTGACCAA (SEQ ID NO: 1052) |
| | | | | | GTTGTCACTCCTAATTCTCAAGTAC (SEQ ID NO: 1053) |
| 201323_at | EBNA1BP2 | EBP2, NOBP, | EBNA1 binding | 10969 | TGGATTTCCTTGAGGGAGATCAGAA (SEQ ID NO: 1054) |
| | | P40 | protein 2 | | AAACCTCTGGCACAGCGCAAGAAGG (SEQ ID NO: 1055) |
| | | | | | GGAGCCAAAGGCCAGCAGATGAGGA (SEQ ID NO: 1056) |
| | | | | | GGCTCAAAGTGGAACACTCGGGAGA (SEQ ID NO: 1057) |
| | | | | | CTTCCGGGCCAAGACAGCTCATGGC (SEQ ID NO: 1058) |
| | | | | | GACAGCTCATGGCAGAGGCCTCAAG (SEQ ID NO: 1059) |
| | | | | | GCCTCAAGAGGCCTGGCAAGAAAGG (SEQ ID NO: 1060) |
| | | | | | GACTCGCAATTTCACGACACACTTT (SEQ ID NO: 1061) |
| | | | | | ACACTTTGATCCCTTCTGTTGGTGT (SEQ ID NO: 1062) |
| | | | | | GAAGTTACTTTCCATTGCTATCTAT (SEQ ID NO: 1063) |
| | | | | | AGACATTGAGTTCAAATTGCCTTCA (SEQ ID NO: 1064) |
| 200789_at | ECH1 | HPXEL | enoyl Coenzyme A | 1891 | ATGTAGGAACACTGGAGCGCCTGCC (SEQ ID NO: 1065) |
| | | | hydratase 1, | | AGGTCATCGGGAACCAGAGCCTGGT (SEQ ID NO: 1066) |
| | | | peroxisomal | | CCAGAGCCTGGTCAACGAGCTGGCC (SEQ ID NO: 1067) |
| | | | | | CCTTCACCGCCCACAAGATGATGGC (SEQ ID NO: 1068) |
| | | | | | GCTGACGAGGCCCTGGACAGTGGGC (SEQ ID NO: 1069) |
| | | | | | TCAGCCGGGTGTTCCCAGACAAAGA (SEQ ID NO: 1070) |
| | | | | | GACCACCGTGTTGGTGCAGAGCACC (SEQ ID NO: 1071) |
| | | | | | GCAGAGCACCAAGGTCAACCTGCTG (SEQ ID NO: 1072) |
| | | | | | GAACATGAGCATGCTGCAGACCCAA (SEQ ID NO: 1073) |
| | | | | | GTCCAGCCCACGACTGAGAACAAGG (SEQ ID NO: 1074) |
| | | | | | GATGACAGTTGTTTCTATGCCTTCT (SEQ ID NO: 1075) |
| 218973_at | EFTUD1 | FAM42A, | elongation factor Tu | 79631 | CTGCAAGTGAAACCTCAGCGCCTGA (SEQ ID NO: 1076) |
| | | FLJ13119, | GTP binding domain | | AGCGCCTGATGGCAGCTATGTACAC (SEQ ID NO: 1077) |
| | | HsT19294, RIA1 | containing 1 | | ACTGGTGATGTTCTCGGTCGAGTCT (SEQ ID NO: 1078) |
| | | | | | GTCGAGTCTATGCTGTCTTGTCAAA (SEQ ID NO: 1079) |
| | | | | | GACATGTTCATCATCAAGGCTGTGC (SEQ ID NO: 1080) |
| | | | | | GCCAGCCCACAACTAGTATTCAGCC (SEQ ID NO: 1081) |
| | | | | | TGGGAGATCATTCCCAGTGACCCCT (SEQ ID NO: 1082) |
| | | | | | CCCTTCTGGGTGCCAACTACTGAGG (SEQ ID NO: 1083) |
| | | | | | TGACTCTGAGAACCAAGCCCGGAAG (SEQ ID NO: 1084) |
| | | | | | AGCTACCTACTACTGGTGGATTCTT (SEQ ID NO: 1085) |
| | | | | | GCCACATTATCTCTGTTTATTCACT (SEQ ID NO: 1086) |
| 201984_s_at | EGFR | Dmel_CG10079, | epidermal growth | 1956 | CTCAAAGAGTATATGTTCCCTCCAG (SEQ ID NO: 1087) |
| | | C-erb, CG10079, | factor receptor | | CCTCCTTACGCTTTGTCACACAAAA (SEQ ID NO: 1088) |
| | | D-EGFR, D-Egf, | (erythroblastic | | AAAGTGTCTCTGCCTTGAGTCATCT (SEQ ID NO: 1089) |
| | | DER, DER flb, | leukemia viral (v-erb- | | GAGTCATCTATTCAAGCACTTACAG (SEQ ID NO: 1090) |
| | | DER/EGFR, | b) oncogene | | GCCACAACAGGGCATTTTACAGGTG (SEQ ID NO: 1091) |
| | | DER/faint little | homolog, avian) | | GTGCCCTGTAACCTGACTGGTTAAC (SEQ ID NO: 1092) |
| | | ball, DER/top, | | | GGTTAACAGCAGTCCTTTGTAAACA (SEQ ID NO: 1093) |
| | | DER/torpedo, | | | TAAACTCTCCTAGTCAATATCCACC (SEQ ID NO: 1094) |
| | | Degfr, DmHD-33, | | | ATCCACCCCATCCAATTTATCAAGG (SEQ ID NO: 1095) |
| | | EFG-R, EGF-R, | | | TCAGCCTACAGTTATGTTCAGTCAC (SEQ ID NO: 1096) |
| | | EK2-6, Egf, El, | | | TGACTCCCAGATCAGTCAGAGCCCC (SEQ ID NO: 1097) |
| | | Elp, Elp-1, Elp- | | | |
| | | B1, Elp-B1RB1, | | | |
| | | HD-33, | | | |
| | | Torpedo/DER, | | | |
| | | Torpedo/Egfr, c- | | | |
| | | erbB, d-egf-r, | | | |
| | | dEGFR1, flb, | | | |
| | | I(2)05351, | | | |
| | | I(2)09261, | | | |
| | | I(2)57DEFa, | | | |
| | | I(2)57EFa, | | | |
| | | I(2)57Ea, pnt, | | | |
| | | top, top/DER, | | | |
| | | top/flb | | | |
| 200595_s_at | EIF3A | EIF-3A | eukaryotic translation | 8661 | GAAGACCCTTTAAAGCAGTGAATCT (SEQ ID NO: 1098) |
| | | | initiation factor 3, | | GAAACAATTTTCACACCCTTAAGTG (SEQ ID NO: 1099) |
| | | | subunit A | | GTGGTTGATACGTACCTATTTTAGG (SEQ ID NO: 1100) |
| | | | | | ATTACTGCGCAAGTGTGAATCCAAA (SEQ ID NO: 1101) |
| | | | | | ATTATCGCTGAGTTTTTTTCTGTTG (SEQ ID NO: 1102) |
| | | | | | ATAGCAAACTGCTTTTCCATTAATG (SEQ ID NO: 1103) |
| | | | | | AATGGAGAATTCATGCCTTTCAAGC (SEQ ID NO: 1104) |
| | | | | | GTAATGACTGTGTTGACTTCAATTT (SEQ ID NO: 1105) |
| | | | | | GACTTCAATTTTGGAGCGCAGTAGC (SEQ ID NO: 1106) |
| | | | | | TGAACTATTTGGTCTCATTGAAGCC (SEQ ID NO: 1107) |
| | | | | | GGTCTCATTGAAGCCAACACAGAAC (SEQ ID NO: 1108) |
| 221094_s_at | ELP3 | FLJ10422, KAT9 | elongation protein 3 | 55140 | GGTGGCTGGGAAACATTCTTGTCAT (SEQ ID NO: 1109) |
| | | | homolog (S. cerevisiae) | | CAAGACATTTTGATTGGCCTCCTAC (SEQ ID NO: 1110) |
| | | | | | GGCCTCCTACGATTACGCAAGTGTT (SEQ ID NO: 1111) |
| | | | | | ATGGGTCTGGGAAAATCGCTGTGAT (SEQ ID NO: 1112) |
| | | | | | GATATCAGGGGTCGGCACCAGGAAT (SEQ ID NO: 1113) |
| | | | | | TCGGCTACAGATTACAAGGCCCGTA (SEQ ID NO: 1114) |
| | | | | | AGATGCTGAAATAATGGCCACACCA (SEQ ID NO: 1115) |
| | | | | | ATCCTCCCTGGCAGAACACGGAGAA (SEQ ID NO: 1116) |
| | | | | | AGAGCAAATGGGGGGCTTCACCCTC (SEQ ID NO: 1117) |
| | | | | | GAGACTGGAAACTGCCTTCAAGGCC (SEQ ID NO: 1118) |
| | | | | | AAATCACTTGCGTTTTTGAGGCTTA (SEQ ID NO: 1119) |
| 213895_at | EMP1 | CL-20, EMP-1, | epithelial membrane | 2012 | AAGGACTGGTATCTTTCTGTGAGCA (SEQ ID NO: 1120) |
| | | TMP | protein 1 | | GATAAAGACTGCATATCCTTGTGTC (SEQ ID NO: 1121) |
| | | | | | ATTGCATTGATTCTTGATGCTTTCT (SEQ ID NO: 1122) |
| | | | | | GATGCTTTCTTAGAGGCCTACATGA (SEQ ID NO: 1123) |
| | | | | | GATTTCTTAGATTGCTCTGATAAAC (SEQ ID NO: 1124) |
| | | | | | AGAGGGAACTTTTGTCAGACTCTGC (SEQ ID NO: 1125) |
| | | | | | CAGACTCTGCAACAAACTCCTAGCT (SEQ ID NO: 1126) |
| | | | | | AACTCCTAGCTCTATCCAGAGTGTC (SEQ ID NO: 1127) |
| | | | | | TCCAGAGTGTCCTCTGCTGCTAAGA (SEQ ID NO: 1128) |
| | | | | | GTATCTTTCTCCTCAAAAGCCTGGA (SEQ ID NO: 1129) |
| | | | | | GGGGGTGCATTAGTCAGAATTCTCC (SEQ ID NO: 1130) |
| 214053_at | ERBB4 | HER4, | v-erb-a erythroblastic | 2066 | ATTGGCCTGTGATTGTTTCTTTTTT (SEQ ID NO: 1131) |
| | | MGC138404, | leukemia viral | | GTGATTCTGTGTTCAGCACTATTGT (SEQ ID NO: 1132) |
| | | p180erbB4 | oncogene homolog 4 | | TGTGATGTGTTCAACCTCTGCACTC (SEQ ID NO: 1133) |
| | | | (avian) | | TTTTAGTATACTTTCTCTTATTCTT (SEQ ID NO: 1134) |
| | | | | | TAGGCTTTTCCGTTAGTTCTCAAGG (SEQ ID NO: 1135) |
| | | | | | TCAAGGATCCTCTTTTGGCTCTTGG (SEQ ID NO: 1136) |
| | | | | | TAGAATGCGAACTGCTTTGCCTCAT (SEQ ID NO: 1137) |
| | | | | | TTGCCTCATTCCATACTGATCATCC (SEQ ID NO: 1138) |
| | | | | | TACTGATCATCCCAGCTGAACAATT (SEQ ID NO: 1139) |
| | | | | | ATTTGAAAACTGTTCTGCCTTTTTG (SEQ ID NO: 1140) |
| | | | | | TCTGCCTTTTTGTTACATGAATCTG (SEQ ID NO: 1141) |
| 201216_at | ERP29 | C12orf8, ERp28, | endoplasmic | 10961 | GACTTTGAGAACCCAGTCCCATACA (SEQ ID NO: 1142) |
| | | ERp31, PDI-DB | reticulum protein 29 | | TTAAGGTTGGAGCCATCCAGCGCTG (SEQ ID NO: 1143) |
| | | | | | TACCTAGGTATGCCTGGTTGCCTGC (SEQ ID NO: 1144) |
| | | | | | TGGCCGGGGAGTTCATCAGGGCCTC (SEQ ID NO: 1145) |
| | | | | | GTGGGCCGAGCAATACCTGAAGATC (SEQ ID NO: 1146) |
| | | | | | GACACGGATCGCCAGGCTGATTGAG (SEQ ID NO: 1147) |
| | | | | | AAGAGCTTAAACATCCTGACTGCCT (SEQ ID NO: 1148) |
| | | | | | AAGGCTGTCTGTGATTTTCCAGGGT (SEQ ID NO: 1149) |
| | | | | | GAGGGGAGAGTTAACCTGCTGGCTG (SEQ ID NO: 1150) |
| | | | | | CTGGCTGTGAGTCCCTTGTGGAATA (SEQ ID NO: 1151) |
| | | | | | TGGTACTAACCCACGATTCTGAGCC (SEQ ID NO: 1152) |
| 221911_at | ETV1 | DKFZp781L0674, | ets variant gene 1 | 2115 | TAATATTCTCTTTCATGCTGAATTT (SEQ ID NO: 1153) |
| | | ER81, | | | TGCTGAATTTACTATGACCATTTAT (SEQ ID NO: 1154) |
| | | MGC104699, | | | TAAGCAGTGCAGTTAACTACAGATA (SEQ ID NO: 1155) |
| | | MGC120533, | | | AACTACAGATAGCATTTCAGGACAA (SEQ ID NO: 1156) |
| | | MGC120534 | | | AGCTTACGCCATGCTATGCTATAAG (SEQ ID NO: 1157) |
| | | | | | GAATATGAGGAAGTTGCTGTATGAC (SEQ ID NO: 1158) |
| | | | | | GCTGTATGACATAGTGCTGGCACTG (SEQ ID NO: 1159) |
| | | | | | GTGCTGGCACTGATATTATCCATCA (SEQ ID NO: 1160) |
| | | | | | CTGATATTATCCATCATCTCTTTTT (SEQ ID NO: 1161) |
| | | | | | GGGTATGTTGTTTCATTCACCTTGA (SEQ ID NO: 1162) |
| | | | | | TAGAATCTCTGAATAGCTCGCTAAA (SEQ ID NO: 1163) |
| 215947_s_at | FAM136A | FLJ14668 | family with sequence | 84908 | AGAGAAGGGGGTTTTGATTGTGATA (SEQ ID NO: 1164) |
| | | | similarity 136, | | GAGCAGTTGGCTACTTGATTACAAC (SEQ ID NO: 1165) |
| | | | member A | | TTGGGCTAGGATGAGCTGACTCCCT (SEQ ID NO: 1166) |
| | | | | | ATGAGCTGACTCCCTTAGAGCAAAG (SEQ ID NO: 1167) |
| | | | | | GCAAAGGAGAGACAGCCCCCATTAC (SEQ ID NO: 1168) |
| | | | | | AAATACCATTTTTGCCTGGGGCTTG (SEQ ID NO: 1169) |
| | | | | | CCAGCATGGCACCTTATTGTTTTGA (SEQ ID NO: 1170) |
| | | | | | AGCAACTTCGTTGAATTTTCACCAA (SEQ ID NO: 1171) |
| | | | | | GAATTTTCACCAACTTATTACTTGA (SEQ ID NO: 1172) |
| | | | | | TATAATATAGCCTGTCCGTTTGCTG (SEQ ID NO: 1173) |
| | | | | | GCTGTGATATATTTTCCTAGTGGTT (SEQ ID NO: 1174) |
| 221774_x_at | FAM48A | RGD1307812 | family with sequence | 55578 | ATACTTCAAGTCTCAGGCCCTTAAA (SEQ ID NO: 1175) |
| | | | similarity 48, member A | | CTTAAATCTACTCCAGCTTCCAGGT (SEQ ID NO: 1176) |
| | | | | | TATTTTTAACACTCTGCAGCAGCAG (SEQ ID NO: 1177) |
| | | | | | GTAGTCCTCAACAGCCAGGGGAGCA (SEQ ID NO: 1178) |
| | | | | | CAAGAACAGGCCTTATCTGCTCAGC (SEQ ID NO: 1179) |
| | | | | | CAGTCACAGGCAGCTGCAGTTGCGA (SEQ ID NO: 1180) |
| | | | | | GCAGTTGCGATTCTTGCAGCATCAA (SEQ ID NO: 1181) |
| | | | | | GCAGCAGCAGCACAAACAGCTCAGC (SEQ ID NO: 1182) |
| | | | | | AGCTCAGCTACATCATCATCGGCAT (SEQ ID NO: 1183) |
| | | | | | TCATCGGCATACAGGCAGCCAGTCA (SEQ ID NO: 1184) |
| | | | | | GTCTTGCATTACTTTTTGTTCCTTT (SEQ ID NO: 1185) |
| 218126_at | FAM82C | RMD3; | family with sequence | 55177 | CTTTTTTCCTTAGACCTTGCTGAGA (SEQ ID NO: 1186) |
| | | FAM82A2; | similarity 82, member C | | ACCACACAAATCTGTCTCCTGGGTC (SEQ ID NO: 1187) |
| | | FAM82C; hRMD- | | | TACCACTCCCCATTAGTTAATTTAT (SEQ ID NO: 1188) |
| | | 3; ptpip51; | | | GTTGTTCTCTCCCTTGAGTAATCTC (SEQ ID NO: 1189) |
| | | FLJ10579 | | | ACCTGCCCCAGGATTACACATGGGT (SEQ ID NO: 1190) |
| | | | | | GGTAGAGCCTGCAAGACCTGAGACC (SEQ ID NO: 1191) |
| | | | | | GACCTGAGACCTTCCAATTGCTGGT (SEQ ID NO: 1192) |
| | | | | | CAGAGTGGCCCACAGACATTGCTTT (SEQ ID NO: 1193) |
| | | | | | GAATTCTACCTGTATTCCAGGGCTG (SEQ ID NO: 1194) |
| | | | | | AGGGCTGGACCACTTGATAACTTCC (SEQ ID NO: 1195) |
| | | | | | AACTTCCAGTGTCCTGGCAGCTTTT (SEQ ID NO: 1196) |
| 210950_s_at | FDFT1 | DGPT, ERG9, | farnesyl-diphosphate | 2222 | GAACTTATAACCAATGCACTGCACC (SEQ ID NO: 1197) |
| | | SQS, SS | farnesyltransferase 1 | | TGCACCACATCCCAGATGTCATCAC (SEQ ID NO: 1198) |
| | | | | | TCACCTACCTTTCGAGACTCAGAAA (SEQ ID NO: 1199) |
| | | | | | TTCCACAGGTGATGGCCATTGCCAC (SEQ ID NO: 1200) |
| | | | | | TTGCCACTTTGGCTGCCTGTTATAA (SEQ ID NO: 1201) |
| | | | | | GATGGATGCCACCAATATGCCAGCT (SEQ ID NO: 1202) |
| | | | | | TTATCATAGAATCCCCGACTCAGAC (SEQ ID NO: 1203) |
| | | | | | CTCAGACCCATCTTCTAGCAAAACA (SEQ ID NO: 1204) |
| | | | | | AACTGTCAGCTGATTTCCCGAAGCC (SEQ ID NO: 1205) |
| | | | | | ACCTGTCGTTTGTCATGCTTTTGGC (SEQ ID NO: 1206) |
| | | | | | TTGTCCATAGCTGAAGTCCACCATA (SEQ ID NO: 1207) |
| 217518_at | FER1L3 | RP11-624L12.2, | fer-1-like 3, myoferlin | 26509 | GGGTGGGTGCTAATGAGCAGGGCCC (SEQ ID NO: 1208) |
| | | FLJ36571, | (C. elegans) | | AGAGCCCTCTTGCATGAGTTTCGGC (SEQ ID NO: 1209) |
| | | FLJ90777, MYOF | | | GTTACGTGACTGCCACATTGGGGCT (SEQ ID NO: 1210) |
| | | | | | CATTGGGGCTTGGAGGCATCTGGCA (SEQ ID NO: 1211) |
| | | | | | GAATGGGCTGGCACCACACTAATTA (SEQ ID NO: 1212) |
| | | | | | AGGCCACGATGATCCAGTTTGACTC (SEQ ID NO: 1213) |
| | | | | | GACAAGCGACTGAGCTAGGCACGGC (SEQ ID NO: 1214) |
| | | | | | CTAGGCACGGCTGACTAGCTCTGAG (SEQ ID NO: 1215) |
| | | | | | AGCTCTGAGCTTTCTGTTCATGTTT (SEQ ID NO: 1216) |
| | | | | | ATTCTTCATTCAATTTTCAGCCTGT (SEQ ID NO: 1217) |
| | | | | | TTCAGCCTGTAGTGTCCTCAAGCAG (SEQ ID NO: 1218) |
| 206404_at | FGF9 | GAF, HBFG-9, | fibroblast growth | 2254 | GTGGGTTCTTATTGATTCGCTGTGT (SEQ ID NO: 1219) |
| | | MGC119914, | factor 9 (glia- | | CTCCACTGTTGCCAAACTTTGTCGC (SEQ ID NO: 1220) |
| | | MGC119915 | activating factor) | | AGGGCCACTTGCTTGATTTATCATG (SEQ ID NO: 1221) |
| | | | | | GAGACTGAGCGCTAGGAGTGTGTGT (SEQ ID NO: 1222) |
| | | | | | CTGCGGCCTGATGCATGCTGGAAAA (SEQ ID NO: 1223) |
| | | | | | GACACGCTTTTCATTTCTGATCAGT (SEQ ID NO: 1224) |
| | | | | | TTCATCCTATATCAGCACAGCTGCC (SEQ ID NO: 1225) |
| | | | | | CAGCTGCCATACTTCGACTTATCAG (SEQ ID NO: 1226) |
| | | | | | ATCAGGATTCTGGCTGGTGGCCTGC (SEQ ID NO: 1227) |
| | | | | | TGCGCGAGGGTGCAGTCTTACTTAA (SEQ ID NO: 1228) |
| | | | | | GTTAATTCTCACTGGTATCATCGCA (SEQ ID NO: 1229) |
| 219390_at | FKBP14 | FKBP22; | FK506 binding | 55033 | CATTGAGACATGCTACCTAGGACTT (SEQ ID NO: 1230) |
| | | FLJ20731 | protein 14, 22 kDa | | GCTACCTAGGACTTAAGCTGATGAA (SEQ ID NO: 1231) |
| | | | | | AGCTGATGAAGCTTGGCTCCTAGTG (SEQ ID NO: 1232) |
| | | | | | TTGGCTCCTAGTGATTGGTGGCCTA (SEQ ID NO: 1233) |
| | | | | | GTGATTGGTGGCCTATTATGATAAA (SEQ ID NO: 1234) |
| | | | | | AATATTTACTTCTTAAGGCTAGCGG (SEQ ID NO: 1235) |
| | | | | | AGGCTAGCGGAATATCCTTCCTGGT (SEQ ID NO: 1236) |
| | | | | | TATCCTTCCTGGTTCTTTAATGGGT (SEQ ID NO: 1237) |
| | | | | | GGTTCTTTAATGGGTAGTCTATAGT (SEQ ID NO: 1238) |
| | | | | | GTAAACCAGTCTACATTTTCCCATT (SEQ ID NO: 1239) |
| | | | | | CTGTCTCATCAAAAACTGAAGTTAG (SEQ ID NO: 1240) |
| 58780_s_at | FLJ10357 | SOLO | hypothetical protein | 55701 | CTGGAGTCCCTGGTGACTCCATTCT (SEQ ID NO: 1241) |
| | | | FLJ10357 | | GAGTCCCTGGTGACTCCATTCTGAG (SEQ ID NO: 1242) |
| | | | | | GTCCCTGGTGACTCCATTCTGAGGT (SEQ ID NO: 1243) |
| | | | | | TCCCTGGTGACTCCATTCTGAGGTG (SEQ ID NO: 1244) |
| | | | | | CTGGTGACTCCATTCTGAGGTGTCA (SEQ ID NO: 1245) |
| | | | | | GGTGACTCCATTCTGAGGTGTCACA (SEQ ID NO: 1246) |
| | | | | | GACTCCATTCTGAGGTGTCACAAGC (SEQ ID NO: 1247) |
| | | | | | CTCCATTCTGAGGTGTCACAAGCAA (SEQ ID NO: 1248) |
| | | | | | TCCATTCTGAGGTGTCACAAGCAAT (SEQ ID NO: 1249) |
| | | | | | ATTCTGAGGTGTCACAAGCAATGAA (SEQ ID NO: 1250) |
| | | | | | TTCTGAGGTGTCACAAGCAATGAAG (SEQ ID NO: 1251) |
| | | | | | TCTGAGGTGTCACAAGCAATGAAGC (SEQ ID NO: 1252) |
| | | | | | TGAGGTGTCACAAGCAATGAAGCTA (SEQ ID NO: 1253) |
| | | | | | AGGTGTCACAAGCAATGAAGCTATG (SEQ ID NO: 1254) |
| | | | | | GTGTGACAGGGGAACCGTAGACTTT (SEQ ID NO: 1255) |
| | | | | | TGTGACAGGGGAACCGTAGACTTTA (SEQ ID NO: 1256) |
| 220912_at | FLJ11827 | FLJ11827 | hypothetical protein | 80163 | CCACCCAAATGTCCTTCAAGGGGCG (SEQ ID NO: 1257) |
| | | | FLJ11827 | | GTGATACATCCATACCATAGACTAC (SEQ ID NO: 1258) |
| | | | | | CATAGACTACTCCCCAGCAATAAAA (SEQ ID NO: 1259) |
| | | | | | AAATCATTCGTGCAACAACTTGGAT (SEQ ID NO: 1260) |
| | | | | | TGCTGAGTGAAAAACACCAATCCTA (SEQ ID NO: 1261) |
| | | | | | GGGCAACACAAGGGTTCCTTATAGT (SEQ ID NO: 1262) |
| | | | | | GTGCCTTGACTGTGGCGATGATTAC (SEQ ID NO: 1263) |
| | | | | | GAACTACCACACAATCAAAGGACTG (SEQ ID NO: 1264) |
| | | | | | AGTGGATGACATCTATCTGAATATC (SEQ ID NO: 1265) |
| | | | | | ATATCCTGGTTGTGACATTGTGGTA (SEQ ID NO: 1266) |
| | | | | | GTAAGGGGTACACGGGATAATTCTG (SEQ ID NO: 1267) |
| 219182_at | FLJ22167 | ALYE870, | hypothetical protein | 79583 | TTACCCTCGTGGCTAAGCAAGTGTC (SEQ ID NO: 1268) |
| | | PRO1886 | FLJ22167 | | GCACACGGAAGATGGCTTGTTCAGC (SEQ ID NO: 1269) |
| | | | | | ACCTCCTGAGGATGTGGGCAGTCTC (SEQ ID NO: 1270) |
| | | | | | GCAGTCTCCTCCAAGAACACATGGA (SEQ ID NO: 1271) |
| | | | | | ATCCCAAGCAGGTCATTGCCACTGG (SEQ ID NO: 1272) |
| | | | | | TGCCACTGGAAGGACATGGCCCCGG (SEQ ID NO: 1273) |
| | | | | | GAAACACACCCCTCAGTGTGTGCCT (SEQ ID NO: 1274) |
| | | | | | GTGCCTCAGTTTACTTTGGAGATCA (SEQ ID NO: 1275) |
| | | | | | GATCAGTTGTCGTTTTTAGTGCTCC (SEQ ID NO: 1276) |
| | | | | | TTTTAGTGCTCCTTTAGGCTTACTA (SEQ ID NO: 1277) |
| | | | | | GCAGAGGAATTCCCTAACACTGACC (SEQ ID NO: 1278) |
| 212546_s_at | FRYL | FLJ16177; | FRY-like | 285527 | AGACCTGCAAAGCTGCTTGGATTTC (SEQ ID NO: 1279) |
| | | KIAA0826; | | | TGAAAACCTAACCTGCAATTGATGC (SEQ ID NO: 1280) |
| | | DKFZp686E205 | | | GATGCTTAATCATCCATCCACATGA (SEQ ID NO: 1281) |
| | | | | | CATGAACATGCAGTCAGCTATACGC (SEQ ID NO: 1282) |
| | | | | | GCTATACGCATCAAGACTCGTTTGC (SEQ ID NO: 1283) |
| | | | | | GGTGATAACGTCTCAGAGCATGCCT (SEQ ID NO: 1284) |
| | | | | | GAGCATGCCTAAAAGAGCACTGCAA (SEQ ID NO: 1285) |
| | | | | | TTGGAATGCTGCTCAGTTCTCTGAT (SEQ ID NO: 1286) |
| | | | | | TTCTCTGATCAGTGCTTATGTTATG (SEQ ID NO: 1287) |
| | | | | | TAACCAAAGTAGATGCCTGCAGAGA (SEQ ID NO: 1288) |
| | | | | | GTATGCTGCCTGTCAGCTATTCTCA (SEQ ID NO: 1289) |
| 219683_at | FZD3 | Fz-3, hFz3 | frizzled homolog 3 | 7976 | AATCCTAAATGTGTGGTGACTGCTT (SEQ ID NO: 1290) |
| | | | (Drosophila) | | GTGACTGCTTTGTAGTGAACTTTCA (SEQ ID NO: 1291) |
| | | | | | AACATTCTGGTAGCTCAGTTAATAA (SEQ ID NO: 1292) |
| | | | | | TTCTATGCAAGGTTTACTTCTCAGA (SEQ ID NO: 1293) |
| | | | | | GAACAGTAGGACTTTGTAGTTTTAT (SEQ ID NO: 1294) |
| | | | | | GAAAGGTTCTCTTAAAATTCTATCG (SEQ ID NO: 1295) |
| | | | | | GATATTCAGGGTTTGGATTAGCAGT (SEQ ID NO: 1296) |
| | | | | | AGAGATGGGCATTGTTTCCCCTATA (SEQ ID NO: 1297) |
| | | | | | GTTTCCCCTATAATTGTGCTGTTTT (SEQ ID NO: 1298) |
| | | | | | TTCTGGCTGTGTTTTTATAACTTAT (SEQ ID NO: 1299) |
| | | | | | TTAATTTGTAGCCATCTTTTCCCAT (SEQ ID NO: 1300) |
| 206335_at | GALNS | FLJ17434, | galactosamine (N- | 2588 | CAGGGGGACTGCACAATTCCACCTG (SEQ ID NO: 1301) |
| | | FLJ42844, | acetyl)-6-sulfate | | GTGAACCCCAAATATCTGAGACCAG (SEQ ID NO: 1302) |
| | | FLJ98217, | sulfatase (Morquio | | GAGACCAGTCTCAGTTTATTTTGCC (SEQ ID NO: 1303) |
| | | GALNAC6S, | syndrome, | | TAAGGATGCACCTGTGACAGCCTCA (SEQ ID NO: 1304) |
| | | GAS, MPS4A | mucopolysaccharidosis | | TACATTGGTTCAGTCAGCCTTCCAC (SEQ ID NO: 1305) |
| | | | type IVA) | | AATACACGATTGAGTCTGGCCCAGT (SEQ ID NO: 1306) |
| | | | | | GCCCAGTGAATCCGCATTTTTATGT (SEQ ID NO: 1307) |
| | | | | | CAGAGGGATGACTTCCAGTTCCGTC (SEQ ID NO: 1308) |
| | | | | | CCGTCTGTCCTTTGTCCACAAGGAA (SEQ ID NO: 1309) |
| | | | | | GAATTTCCCTGGGCGCTAATTATGA (SEQ ID NO: 1310) |
| | | | | | GAGGCGTGTAGCTTCTTATCATTGT (SEQ ID NO: 1311) |
| 214718_at | GATAD1 | tcag7.279, | GATA zinc finger | 57798 | GCTGGGCAATGGAGTCAGATTCTCT (SEQ ID NO: 1312) |
| | | FLJ22489, | domain containing 1 | | GGAGTCAGATTCTCTTTCTTAAAAA (SEQ ID NO: 1313) |
| | | FLJ40695, | | | ACTGGATTTCCAGTTCTCTAATATT (SEQ ID NO: 1314) |
| | | ODAG, | | | GTTCTCTAATATTCTTAGTACCACA (SEQ ID NO: 1315) |
| | | RG083M05.2 | | | TTAGTACCACAAGATATGTCATAGG (SEQ ID NO: 1316) |
| | | | | | GAAATTCTTAGCTGGAAAAGTGACT (SEQ ID NO: 1317) |
| | | | | | TTTTTCTCCTGCTACCTAGTAATAA (SEQ ID NO: 1318) |
| | | | | | TCTCCTGCTACCTAGTAATAAACAA (SEQ ID NO: 1319) |
| | | | | | GTTTATTACTGGTCACTTAGAAAAT (SEQ ID NO: 1320) |
| | | | | | ACCTGAGGTCGGGAAGTGGATCGCC (SEQ ID NO: 1321) |
| | | | | | AGGTCGGGAAGTGGATCGCCTGAGG (SEQ ID NO: 1322) |
| 204867_at | GCHFR | GFRP, | GTP cyclohydrolase I | 2644 | TGGGCCCCACTATGGTGGGCGATGA (SEQ ID NO: 1323) |
| | | HsT16933, | feedback regulator | | GAACAGTCGGATCCAGAGCTGATGC (SEQ ID NO: 1324) |
| | | MGC138467, | | | ATGAATACTACGTCGATGACCCTCC (SEQ ID NO: 1325) |
| | | MGC138469, | | | TCCCCGCATAGTCCTGGACAAGCTG (SEQ ID NO: 1326) |
| | | P35 | | | CAAGCTGGAACGCAGGGGCTTCCGT (SEQ ID NO: 1327) |
| | | | | | TGCTGAGCATGACGGGGGTGGGCCA (SEQ ID NO: 1328) |
| | | | | | GCCAGACGCTGGTGTGGTGTCTGCA (SEQ ID NO: 1329) |
| | | | | | GGTGTCTGCACAAGGAGTGACCTTC (SEQ ID NO: 1330) |
| | | | | | GACCTTCTCATGCTGATTTGCAGAC (SEQ ID NO: 1331) |
| | | | | | GGCAGTGAATGGCCTTCTCTGAAAC (SEQ ID NO: 1332) |
| | | | | | TTCTCTGAAACCCTGCGTCAAGCAG (SEQ ID NO: 1333) |
| 209248_at | GHITM | DERP2; MICS1; | growth hormone | 27069 | AATTTAGTAGGTTCACTGAGTAACT (SEQ ID NO: 1334) |
| | | My021; PTD010; | inducible | | GAAGGAGTCACCTGCAGTCTTTTGT (SEQ ID NO: 1335) |
| | | TMBIM5; | transmembrane | | AATACTTAGAACTTAGCACTTGTGT (SEQ ID NO: 1336) |
| | | HSPC282; | protein | | GTTATTGATTAGTGAGGAGCCAGTA (SEQ ID NO: 1337) |
| | | FLJ26584; | | | GAAACATCTGGGTATTTGGAAACAA (SEQ ID NO: 1338) |
| | | DKFZp566C0746 | | | GTGGTCATTGTTACATTCATCTGCT (SEQ ID NO: 1339) |
| | | | | | TCATCTGCTGAACTTAACAAAACTG (SEQ ID NO: 1340) |
| | | | | | ACAGGCACAGGTGATGCATTCTCCT (SEQ ID NO: 1341) |
| | | | | | ACAGGTGATGCATTCTCCTGCTGTC (SEQ ID NO: 1342) |
| | | | | | CTCAGTGCTCTCTTTCCAATATAGA (SEQ ID NO: 1343) |
| | | | | | ATGTGGTCATGTTTGACTTGTACAG (SEQ ID NO: 1344) |
| 215243_s_at | GJB3 | CX31, DFNA2, | gap junction protein, | 2707 | CACTGCTAGCAGGGCTTCAACCAGG (SEQ ID NO: 1345) |
| | | EKV, FLJ22486, | beta 3, 31 kDa | | GGAGAGGCTTCCCTGAGGACATAAT (SEQ ID NO: 1346) |
| | | MGC102938 | | | AGGTGAGAAGTGCTCCCAAGCAGAC (SEQ ID NO: 1347) |
| | | | | | CCAAGCAGACACAACAGCAGCACAG (SEQ ID NO: 1348) |
| | | | | | AGCAGCACAGAGGTCTGGAGGCCAC (SEQ ID NO: 1349) |
| | | | | | ACACAAAAAGTGATGCTCGCCCTGG (SEQ ID NO: 1350) |
| | | | | | GGCTAGCCTCAGCAGACCTAAGGCA (SEQ ID NO: 1351) |
| | | | | | AGCAGACCTAAGGCATCTCTACTCC (SEQ ID NO: 1352) |
| | | | | | TCTGGAGATACCCAGAGGTCCCCCA (SEQ ID NO: 1353) |
| | | | | | CCAGGTCATCACTTGGCTCAGTGGA (SEQ ID NO: 1354) |
| | | | | | ACTTGGCTCAGTGGAAGCCCTCTTT (SEQ ID NO: 1355) |
| 209092_s_at | GLOD4 | C17orf25, CGI- | glyoxalase domain | 51031 | AAATCCAATCATGAGTCCAGGTAGA (SEQ ID NO: 1356) |
| | | 150, HC71 | containing 4 | | AGGTAGAGAACGCCTGCTGTAATCT (SEQ ID NO: 1357) |
| | | | | | GACTGCGCATTCTGTATATAACTGT (SEQ ID NO: 1358) |
| | | | | | TCCAGACTAGGACAGCGGCATGAAC (SEQ ID NO: 1359) |
| | | | | | ATGACTTTGGTTGGGATTGCGGATA (SEQ ID NO: 1360) |
| | | | | | GATTGCGGATAGTTAGGGTTACCTC (SEQ ID NO: 1361) |
| | | | | | GGGTTACCTCTGAATCGTGTAGCTT (SEQ ID NO: 1362) |
| | | | | | GAATCCACATTAATGCCTTGTCGTG (SEQ ID NO: 1363) |
| | | | | | CCTGACGATACGCTCTTCTATTGTC (SEQ ID NO: 1364) |
| | | | | | GCTCTTCTATTGTCTTATTCTGGCA (SEQ ID NO: 1365) |
| | | | | | TGTGCGAGCTTCTTTCTGTGTATAT (SEQ ID NO: 1366) |
| 221510_s_at | GLS | AAD20, | glutaminase | 2744 | GAAGATTTAGTTTTCATATCGTATA (SEQ ID NO: 1367) |
| | | DKFZp686O15119, | | | AATTTGTGTCACTTACTATTTTTGT (SEQ ID NO: 1368) |
| | | FLJ10358, | | | GTACTGTTATCTAAGCTACTGTGTT (SEQ ID NO: 1369) |
| | | GLS1, KIAA0838 | | | AGATACTGCGAATAGGCCCTCAAAC (SEQ ID NO: 1370) |
| | | | | | TTTGCCAGTTTTAAGGACATATTTT (SEQ ID NO: 1371) |
| | | | | | CACTATTATTGGGTTTGTTTTATGC (SEQ ID NO: 1372) |
| | | | | | ATGCCATTATTGATTCTTGATATTC (SEQ ID NO: 1373) |
| | | | | | ACAATGTAGCATATTTGATTTTCTT (SEQ ID NO: 1374) |
| | | | | | TTGTGTTGTTTAGCTTTCATTTGCT (SEQ ID NO: 1375) |
| | | | | | ATGTACCTTTATTTTCCAGTATGCC (SEQ ID NO: 1376) |
| | | | | | TTTCCAGTATGCCTACATTTTGTAT (SEQ ID NO: 1377) |
| 201180_s_at | GNAI3 | RP23-89M15.4, | guanine nucleotide | 2773 | TATCCTGCTCAAAGTACCATTATGG (SEQ ID NO: 1378) |
| | | AI158965, | binding protein (G | | TAAGATTCTAGTTCTCTCTTTTCTT (SEQ ID NO: 1379) |
| | | AW537698, | protein), alpha | | CAGCTTCTTGGATACAGACACACCC (SEQ ID NO: 1380) |
| | | Galphai3, Gnai-3 | inhibiting activity | | GAACCAGTGACTTTGCTGCTACATA (SEQ ID NO: 1381) |
| | | | polypeptide 3 | | TTGACCACTTTGATTCTTGCTTGTT (SEQ ID NO: 1382) |
| | | | | | CTTGCTTGTTTGTCTCAGTTATAGG (SEQ ID NO: 1383) |
| | | | | | TTTGTACATGGTGCCTCTTGTTTTC (SEQ ID NO: 1384) |
| | | | | | AAATGTGGTGCCAAATCCCTGTTTG (SEQ ID NO: 1385) |
| | | | | | TTGCCATCGTTTTTACTGTACCGAT (SEQ ID NO: 1386) |
| | | | | | GTAATCCTTAGCCAGTATGTTCTTT (SEQ ID NO: 1387) |
| | | | | | GAAACTGTTGCATTTTGGGACTTTT (SEQ ID NO: 1388) |
| 221958_s_at | GPR177 | EVI; MRP; WLS; | G protein-coupled | 79971 | GGCTTCACCAAGGTGTGGTTTGCCA (SEQ ID NO: 1389) |
| | | C1orf139; | receptor 177 | | TAGATTTTAACATCCTTTGCGGTCC (SEQ ID NO: 1390) |
| | | FLJ23091; | | | CGGTCCCAGTCCAAGGTAGGCTGGT (SEQ ID NO: 1391) |
| | | MGC14878; | | | CAAGGTAGGCTGGTGTCATAGTCTT (SEQ ID NO: 1392) |
| | | MGC131760 | | | TAATCCATGACCACTGTTTTTTTCC (SEQ ID NO: 1393) |
| | | | | | TTCCTATTTATATCACCAGGTAGCC (SEQ ID NO: 1394) |
| | | | | | GTCAATAGATAAGTGTCCCTGTTTT (SEQ ID NO: 1395) |
| | | | | | TGAGAAGATTTATTCCACCAGGGGT (SEQ ID NO: 1396) |
| | | | | | TCCACCAGGGGTATTTCAGCTTTGA (SEQ ID NO: 1397) |
| | | | | | GAAACCAAATCTGTGTATCTAATAC (SEQ ID NO: 1398) |
| | | | | | ATGTTTGCTAAACTACCCAAGTAAG (SEQ ID NO: 1399) |
| 209409_at | GRB10 | GRB-IR, Grb-10, | growth factor | 2887 | TGTATCTCTGCCTGTGATTTTCTTT (SEQ ID NO: 1400) |
| | | IRBP, KIAA0207, | receptor-bound | | GAAACTCCATGTAGCAACCAGGACG (SEQ ID NO: 1401) |
| | | MEG1, RSS | protein 10 | | TGTGCCTCAGCCTAGATGTTTTGTC (SEQ ID NO: 1402) |
| | | | | | TTCTCTTTTCTGCACTTAATACCTG (SEQ ID NO: 1403) |
| | | | | | TGACAGTATGACCGATCTCTGCGCC (SEQ ID NO: 1404) |
| | | | | | CGCCTTTCTGGGGGCGGGCAAGCTG (SEQ ID NO: 1405) |
| | | | | | GCTGGCGGTAGATTTGTGATGTCAC (SEQ ID NO: 1406) |
| | | | | | AACCTTTATTCCATGTGCTTTGCTT (SEQ ID NO: 1407) |
| | | | | | CTTCATTCTGTACATAGCTCTTTGG (SEQ ID NO: 1408) |
| | | | | | AGCTCTTTGGCTCGTGAACCTAATT (SEQ ID NO: 1409) |
| | | | | | AGGGACTGATGTTCTGTTTCTTGTA (SEQ ID NO: 1410) |
| 220794_at | GREM2 | PRDC; DAND3; | gremlin 2, cysteine | 64388 | CGCCCCCTATTTGTGGACTAAAGAT (SEQ ID NO: 1411) |
| | | CKTSF1B2 | knot superfamily, | | GATGAACTCTGGTGTGCATGCTATT (SEQ ID NO: 1412) |
| | | | homolog (Xenopus | | AAGGTCTCTATATTAACGCTGGTTT (SEQ ID NO: 1413) |
| | | | laevis) | | TCACATGTTTTGACTCTGGCTTGCA (SEQ ID NO: 1414) |
| | | | | | TGGCTTGCAGCACCATTCGGAGTAA (SEQ ID NO: 1415) |
| | | | | | GGAGTAAGGATGACAGCAGGCCCAG (SEQ ID NO: 1416) |
| | | | | | AGCAGGCCCAGAAGGTGTTTTACTC (SEQ ID NO: 1417) |
| | | | | | CAGCAATTCACTGTCATGCACCTAT (SEQ ID NO: 1418) |
| | | | | | TTCACTGACGTTACTAAGCATTGCA (SEQ ID NO: 1419) |
| | | | | | AAACCGTATACTACTTTCACTGCCA (SEQ ID NO: 1420) |
| | | | | | CTACTTTCACTGCCATGACACTAGG (SEQ ID NO: 1421) |
| 216308_x_at | GRHPR | RP11-397D12.2, | glyoxylate | 9380 | CAATCTGATTTCATCGTCGTGGCCT (SEQ ID NO: 1422) |
| | | GLXR, PH2 | reductase/hydroxypyruvate | | TAACACCTGCAACCGAGGGACTCTG (SEQ ID NO: 1423) |
| | | | reductase | | GACTCTGCAACAAGGACTTCTTCCA (SEQ ID NO: 1424) |
| | | | | | CAACATCAGCAGGGGCGACGTCGTA (SEQ ID NO: 1425) |
| | | | | | CCTGTACCAGGCCTTGGCCAGTGGT (SEQ ID NO: 1426) |
| | | | | | GATTCTGCCCCACATTGGCAGTGCC (SEQ ID NO: 1427) |
| | | | | | CACCATGTCCTTGTTGGCAGCTAAC (SEQ ID NO: 1428) |
| | | | | | ACAACTTGCTGGCTGGCCTGAGAGG (SEQ ID NO: 1429) |
| | | | | | ACAGTACAGGTGGAGGGCCGGGAAG (SEQ ID NO: 1430) |
| | | | | | GCAAACCGTGCCCTGGTATTGTCAG (SEQ ID NO: 1431) |
| | | | | | GACACACCCAGGCTTGATTTGGATC (SEQ ID NO: 1432) |
| 206326_at | GRP | BN, GRP-10, | gastrin-releasing | 2922 | AGAGCACAGGGGAGTCTTCTTCTGT (SEQ ID NO: 1433) |
| | | preproGRP, | peptide | | CCTTGGGCAATCAGCAGCCTTCGTG (SEQ ID NO: 1434) |
| | | proGRP | | | CAAAGTTGGTAGACTCTCTGCTCCA (SEQ ID NO: 1435) |
| | | | | | CTGCTCCAGGTTCTCAACGTGAAGG (SEQ ID NO: 1436) |
| | | | | | AACCCCCAGCTGAACCAGCAATGAT (SEQ ID NO: 1437) |
| | | | | | AGCAATGATAATGATGGCCTCTCTC (SEQ ID NO: 1438) |
| | | | | | GCAAGCATCAGTTCTACGGATCATC (SEQ ID NO: 1439) |
| | | | | | TATTCTGTATCTTTCATCCTTGACT (SEQ ID NO: 1440) |
| | | | | | CGTGATTTTCAAGCAGCATCTTCTG (SEQ ID NO: 1441) |
| | | | | | GCTTTTTTATATCTAGGCTACCTGT (SEQ ID NO: 1442) |
| | | | | | GTTGGTTAGATTCAAGGCCCCGAGC (SEQ ID NO: 1443) |
| 201520_s_at | GRSF1 | FLJ13125 | G-rich RNA | 2926 | TCTTCTCTGCATTTTGTCCACATGA (SEQ ID NO: 1444) |
| | | | sequence binding | | TGAGAGGATTACCTTTCCAAGCCAA (SEQ ID NO: 1445) |
| | | | factor 1 | | TAAACTTTTTTGCTCCACTCAAGCC (SEQ ID NO: 1446) |
| | | | | | TCAAGCCTGTTAGAATCACCATGGA (SEQ ID NO: 1447) |
| | | | | | GGAATACAGCTCCAGTGGGAAGGCC (SEQ ID NO: 1448) |
| | | | | | AGCTGATGTGCACTTTGAGACCCAT (SEQ ID NO: 1449) |
| | | | | | GAGGATGCTGTTGCAGCGATGCTCA (SEQ ID NO: 1450) |
| | | | | | GCAGCGATGCTCAAGGATCGGTCCC (SEQ ID NO: 1451) |
| | | | | | GGTCCCACGTTCATCATAGGTATAT (SEQ ID NO: 1452) |
| | | | | | AAGACTCTAGGGGCTCCAGATAATA (SEQ ID NO: 1453) |
| | | | | | TTTGCACATCTTTCTTGGACTTGGG (SEQ ID NO: 1454) |
| 205770_at | GSR | MGC78522 | glutathione reductase | 2936 | TACCAACGTCAAAGGCATCTATGCA (SEQ ID NO: 1455) |
| | | | | | GCAATAGCTGCTGGCCGAAAACTTG (SEQ ID NO: 1456) |
| | | | | | AACTTGCCCATCGACTTTTTGAATA (SEQ ID NO: 1457) |
| | | | | | CAACATCCCAACTGTGGTCTTCAGC (SEQ ID NO: 1458) |
| | | | | | TATTCAACGAGCTTTACCCCGATGT (SEQ ID NO: 1459) |
| | | | | | TTACCCCGATGTATCACGCAGTTAC (SEQ ID NO: 1460) |
| | | | | | AAATGCTGCAGGGTTTTGCTGTTGC (SEQ ID NO: 1461) |
| | | | | | TTTGACAACACAGTCGCCATTCACC (SEQ ID NO: 1462) |
| | | | | | TTCACCCTACCTCTTCAGAAGAGCT (SEQ ID NO: 1463) |
| | | | | | AAGAGCTGGTCACACTTCGTTGAGA (SEQ ID NO: 1464) |
| | | | | | GAACCAGGAGACACGTGTGGCGGGC (SEQ ID NO: 1465) |
| 215766_at | GSTA1 | GST2, GSTA1-1, | Glutathione S- | 2938 | CCCAATGCCACAAAACCACATCAGA (SEQ ID NO: 1466) |
| | | GTH1, | transferase A1 | | ACTGTGCCTGGCTACATTTAGATCT (SEQ ID NO: 1467) |
| | | MGC131939 | | | GAGCATCTTTCCTCTGTTGAGTAGT (SEQ ID NO: 1468) |
| | | | | | GTGGATTCTCTATTTTATTGGTCAT (SEQ ID NO: 1469) |
| | | | | | CAAGGCTAGTGGGCTTTCACACTCC (SEQ ID NO: 1470) |
| | | | | | CACACTCCAGACTGCTGTATTCCAG (SEQ ID NO: 1471) |
| | | | | | CTGTATTCCAGCCCAAATTACTCAA (SEQ ID NO: 1472) |
| | | | | | AGCTAATGCAATCCGCTTGCCTTAC (SEQ ID NO: 1473) |
| | | | | | CTCAGGTTGCTGTTACTCTGTTTCA (SEQ ID NO: 1474) |
| | | | | | CTCTGTTTCAGATGCAACCCTCTGT (SEQ ID NO: 1475) |
| | | | | | CCCAAGTTCTCTCATTCTTAGCTAT (SEQ ID NO: 1476) |
| 205752_s_at | GSTM5 | GSTM5-5, GTM5 | glutathione S- | 2949 | CCTAGCACTGTCCTCAAAGACCATC (SEQ ID NO: 1477) |
| | | | transferase M5 | | CTCAAAGACCATCTGTATGCCCTGC (SEQ ID NO: 1478) |
| | | | | | AGCTCCGTGTGATGCCCAGTAAAGC (SEQ ID NO: 1479) |
| | | | | | TGCCCAGTAAAGCCTGAACCATGCC (SEQ ID NO: 1480) |
| | | | | | CTGAGGCTCCCTTGAACTCAGGACT (SEQ ID NO: 1481) |
| | | | | | GGGGTGGTGATCAGGCAGGTTCATA (SEQ ID NO: 1482) |
| | | | | | CTCCTGTAGGCCGTCTAGAACTGGC (SEQ ID NO: 1483) |
| | | | | | CTCCCAGGCAGACTGCCTTCTTTCA (SEQ ID NO: 1484) |
| | | | | | TCTTTCACCCTGTCCTGATAGACTT (SEQ ID NO: 1485) |
| | | | | | AGACTTCCCTGATCTAGATATCCTT (SEQ ID NO: 1486) |
| | | | | | ATCTAGATATCCTTCGTCATGACAC (SEQ ID NO: 1487) |
| 211555_s_at | GUCY1B3 | GC-S-beta-1, | guanylate cyclase 1, | 2983 | GTTTACCAGAGCCATGCATTCACCA (SEQ ID NO: 1488) |
| | | GC-SB3, | soluble, beta 3 | | TGCATTCACCATGCACGATCCATCT (SEQ ID NO: 1489) |
| | | GUC1B3, | | | GTGTCATAGGACAGCGGATGCCTCG (SEQ ID NO: 1490) |
| | | GUCB3, | | | ACAGCGGATGCCTCGATACTGTCTT (SEQ ID NO: 1491) |
| | | GUCSB3, | | | TGGGAATACTGTCAACCTCACAAGC (SEQ ID NO: 1492) |
| | | GUCY1B1 | | | ACCTCACAAGCCGAACAGAAACCAC (SEQ ID NO: 1493) |
| | | | | | AATTCAGATCCACAATTCCACTTGG (SEQ ID NO: 1494) |
| | | | | | TCCACTTGGAGCACAGAGGCCCAGT (SEQ ID NO: 1495) |
| | | | | | ATGCAAGTTTGGTTTCTATCCAGAA (SEQ ID NO: 1496) |
| | | | | | ACAGACTAGGTTCCAGTTTTCTCCT (SEQ ID NO: 1497) |
| | | | | | CCAGGAGCAGTTCTTCCCTATGGAT (SEQ ID NO: 1498) |
| 200853_at | H2AFZ | H2A.z, H2A/z, | H2A histone family, | 3015 | ACCTCACCGCAGAGGTACTTGAACT (SEQ ID NO: 1499) |
| | | H2AZ, | member Z | | GAGGTACTTGAACTGGCAGGAAATG (SEQ ID NO: 1500) |
| | | MGC117173 | | | GAACTGGCAGGAAATGCATCAAAAG (SEQ ID NO: 1501) |
| | | | | | AGAATTGGATTCTCTCATCAAGGCT (SEQ ID NO: 1502) |
| | | | | | GGATTCTCTCATCAAGGCTACAATT (SEQ ID NO: 1503) |
| | | | | | CTCTCATCAAGGCTACAATTGCTGG (SEQ ID NO: 1504) |
| | | | | | TCAAGGCTACAATTGCTGGTGGTGG (SEQ ID NO: 1505) |
| | | | | | AGTGTTGGTGATTCCAGTGGACTGT (SEQ ID NO: 1506) |
| | | | | | GTGATTCCAGTGGACTGTATCTCTG (SEQ ID NO: 1507) |
| | | | | | ACATTTGGTTTTGTTCAGACCTTAT (SEQ ID NO: 1508) |
| | | | | | GTTTTGTTCAGACCTTATTTCCACT (SEQ ID NO: 1509) |
| 201036_s_at | HADH | HAD, HADH1, | hydroxyacyl- | 3033 | CTTGCCCTACATTTTGGGCATGACA (SEQ ID NO: 1510) |
| | | HADHSC, HHF4, | Coenzyme A | | AGATGTGTCTTTATTCAGCTCGTCG (SEQ ID NO: 1511) |
| | | M/SCHAD, | dehydrogenase | | TTCAGCTCGTCGTGAAGATGCTGCT (SEQ ID NO: 1512) |
| | | MGC8392, | | | CTGAATGGGTCAGCATATCTCTGTT (SEQ ID NO: 1513) |
| | | SCHAD | | | GCATATCTCTGTTTGCATGGTTTGC (SEQ ID NO: 1514) |
| | | | | | CATGGTTTGCAGGAGGTCGGTTTTC (SEQ ID NO: 1515) |
| | | | | | GCAGGAGGTCGGTTTTCATGGTCAT (SEQ ID NO: 1516) |
| | | | | | ATGGTCATTCAGTTCCACAGATCTG (SEQ ID NO: 1517) |
| | | | | | TGAATGATTACTGTCTGTCTGTGTC (SEQ ID NO: 1518) |
| | | | | | TTTCCATGAGAAATCACTGTTGCAA (SEQ ID NO: 1519) |
| | | | | | ATTGCCTATAAATTGACTCTACTAA (SEQ ID NO: 1520) |
| 202299_s_at | HBXIP | MGC71071, XIP | hepatitis B virus x | 10542 | TCTGACCCCACTGATATTCCTGTGG (SEQ ID NO: 1521) |
| | | | interacting protein | | ACCCCACTGATATTCCTGTGGTGTG (SEQ ID NO: 1522) |
| | | | | | GATCCAGAAACACGATGGCATCACG (SEQ ID NO: 1523) |
| | | | | | TGGCATCACGGTGGCAGTGCACAAA (SEQ ID NO: 1524) |
| | | | | | TCAGCAGCCTGTCATAGGAACTGGA (SEQ ID NO: 1525) |
| | | | | | ACTGGATCCTACCTATGTTAATTAC (SEQ ID NO: 1526) |
| | | | | | TGTTAATTACCTTATAGAACTACTA (SEQ ID NO: 1527) |
| | | | | | AAGTTCCAGTAGTTAGGCCATTCAT (SEQ ID NO: 1528) |
| | | | | | TAGTTAGGCCATTCATTTAATGTGC (SEQ ID NO: 1529) |
| | | | | | TAATGTGCATTAGGCACTTTTCTGT (SEQ ID NO: 1530) |
| | | | | | ATGTTTTGAAGCAGCAGGTCCAGGT (SEQ ID NO: 1531) |
| 208826_x_at | HINT1 | FLJ30414, | histidine triad | 3094 | CCCGCGCGGGAGAGAGGCCGAGATG (SEQ ID NO: 1532) |
| | | FLJ32340, HINT, | nucleotide binding | | GAGATTGCCAAGGCTCAGGTCGCTC (SEQ ID NO: 1533) |
| | | PKCI-1, | protein 1 | | CTGGTGGCGACACGATCTTTGGGAA (SEQ ID NO: 1534) |
| | | PRKCNH1 | | | TTTTGAGGATGACCGGTGCCTTGCT (SEQ ID NO: 1535) |
| | | | | | CCTCAAGCACCAACACATTTTCTGG (SEQ ID NO: 1536) |
| | | | | | AGAAATGTGCTGCTGATCTGGGCCT (SEQ ID NO: 1537) |
| | | | | | TGGACAGTCTGTCTATCACGTTCAT (SEQ ID NO: 1538) |
| | | | | | TCACGTTCATCTCCATGTTCTTGGA (SEQ ID NO: 1539) |
| | | | | | CAAATGCATTGGCCTCCTGGTTAAG (SEQ ID NO: 1540) |
| | | | | | GGGATAATTTTCTCTTCTTTAGGCA (SEQ ID NO: 1541) |
| | | | | | AAGTAACACACTTATTTTTGCCTGT (SEQ ID NO: 1542) |
| 216526_x_at | HLA-B /// | CDABP0067, | major | 3106 | CCATCATGGGCATCGTTGCTGGCCT (SEQ ID NO: 1543) |
| | HLA-C /// | AS, HLA-B-7301, | histocompatibility | 3107 | AGCTGTGGTCACCGCTATGATGTGT (SEQ ID NO: 1544) |
| | MICA /// | HLA-B73, HLAB, | complex, class I, B /// | 4276 | GAAGAGCTCAGGTGGTGTGTGGGAC (SEQ ID NO: 1545) |
| | MICB /// | HLAC, SPDA1 /// | major | 4277 | AGATGCAGGATTTCTTCACACCTCT (SEQ ID NO: 1546) |
| | XXbac- | XXbac- | histocompatibility | 100129668 | GTGACTTCAAGAGCCTCTGGCATCT (SEQ ID NO: 1547) |
| | BPG181B23.1 | BCX101P6.2, | complex, class I, C /// | | GCATCTCTTTCTGCAAAGGCGTCTG (SEQ ID NO: 1548) |
| | | D6S204, | MHC class I | | AAGGCGTCTGAATGTGTCTGCGTTC (SEQ ID NO: 1549) |
| | | FLJ27082, HLA- | polypeptide-related | | CGATCATCTTTCCTGTTCCAGAGAG (SEQ ID NO: 1550) |
| | | Cw, HLA-Cw12, | sequence A /// MHC | | AGAGGTGGGGCTGGATGTCTCCATC (SEQ ID NO: 1551) |
| | | HLA-JY3, HLC-C, | class I polypeptide- | | TCTCCATCTCTGTCTCAAATTCATG (SEQ ID NO: 1552) |
| | | PSORS1 /// | related sequence B | | GCACTGAGCTGCAACTTCTTACTTC (SEQ ID NO: 1553) |
| | | DAMA-345G11.2, | /// MHC class I | | |
| | | MGC111087, | polypeptide-related | | |
| | | PERB11.1 /// | sequence A | | |
| | | DAAP-210H10.1, | | | |
| | | PERB11.2 | | | |
| | | /// MICA | | | |
| 213356_x_at | HNRNPA1 | HNRPA1, | heterogeneous | 3178 | AACTACTCATGTGTATGGCGCCGAT (SEQ ID NO: 1554) |
| | /// | MGC1028350 /// | nuclear | 664709 | GGCGCCGATAAATCTCGAGGACTGT (SEQ ID NO: 1555) |
| | HNRPA1L-2 | /// /// /// /// /// /// /// | ribonucleoprotein A1 | 664721 | GGTTATTGCTAGTTTCTGTTCTGTG (SEQ ID NO: 1556) |
| | /// | /// /// /// /// /// /// | /// heterogeneous | 100128836 | TTGCACCCCATGCTGTTGATTGCTA (SEQ ID NO: 1557) |
| | HNRPA1P5 | LOC144983; | nuclear | 391670 | GTAACAGTCTGATCGTGACGCTGAA (SEQ ID NO: 1558) |
| | /// | MGC102957 | ribonucleoprotein A1 | 402112 | AGTTAGTCTACTCTTAAGCCATCTT (SEQ ID NO: 1559) |
| | LOC100128 | | pseudogene /// | 440125 | GCCATCTTGGTAAATTTCCCCAACA (SEQ ID NO: 1560) |
| | 836 /// | | heterogeneous | 642817 | GAATTCCTTCAGGGTGATGCCAGGT (SEQ ID NO: 1561) |
| | LOC391670 | | nuclear | 643033 | AAGCCATTGTCTTCGGAAACCTTGG (SEQ ID NO: 1562) |
| | /// | | ribonucleoprotein A1 | 644037 | GTGATTGTTGGCACATCCTATGCAA (SEQ ID NO: 1563) |
| | LOC402112 | | pseudogene 5 /// | 645001 | GAAGCTTGTGTATCCATTATCATGT (SEQ ID NO: 1564) |
| | /// | | similar to | 728732 | |
| | LOC440125 | | heterogeneous | 729366 | |
| | /// | | nuclear | 729423 | |
| | LOC642817 | | ribonucleoprotein A1 | 730246 | |
| | /// | | /// heterogeneous | 144983 | |
| | LOC643033 | | nuclear | | |
| | /// | | ribonucleoprotein A1- | | |
| | LOC644037 | | like /// similar to | | |
| | /// | | heterogeneous | | |
| | LOC645001 | | nuclear | | |
| | /// | | ribonucleoprotein A1 | | |
| | LOC728170 | | /// similar to | | |
| | /// | | heterogeneous | | |
| | LOC728732 | | nuclear | | |
| | /// | | ribonucleoprotein A1 | | |
| | LOC729366 | | /// similar to | | |
| | /// | | heterogeneous | | |
| | LOC729423 | | nuclear | | |
| | /// | | ribonucleoprotein A1 | | |
| | LOC730246 | | /// hypothetical | | |
| | /// RP11- | | LOC642817 /// | | |
| | 78J21.1 | | similar to | | |
| | | | Heterogeneous | | |
| | | | nuclear | | |
| | | | ribonucleoprotein A1 | | |
| | | | (Helix-destabilizing | | |
| | | | protein) (Single- | | |
| | | | strand binding | | |
| | | | protein) (hnRNP core | | |
| | | | protein A1) (HDP-1) | | |
| | | | (Topoisomerase- | | |
| | | | inhibitor suppressed) | | |
| | | | /// hypothetical | | |
| | | | LOC644037 /// | | |
| | | | similar to | | |
| | | | heterogeneous | | |
| | | | nuclear | | |
| | | | ribonucleoprotein A1 | | |
| | | | /// similar to | | |
| | | | heterogeneous | | |
| | | | nuclear | | |
| | | | ribonucleoprotein A1 | | |
| | | | /// similar to | | |
| | | | heterogeneous | | |
| | | | nuclear | | |
| | | | ribonucleoprotein A1 | | |
| | | | /// similar to | | |
| | | | heterogeneous | | |
| | | | nuclear | | |
| | | | ribonucleoprotein A1 | | |
| | | | /// similar to | | |
| | | | Heterogeneous | | |
| | | | nuclear | | |
| | | | ribonucleoprotein A1 | | |
| | | | (Helix-destabilizing | | |
| | | | protein) (Single- | | |
| | | | strand RNA-binding | | |
| | | | protein) (hnRNP core | | |
| | | | protein A1) (HDP) /// | | |
| | | | hypothetical | | |
| | | | LOC730246 /// | | |
| | | | heterogeneous | | |
| | | | nuclear | | |
| | | | ribonucleoprotein A1- | | |
| | | | like | | |
| 206289_at | HOXA4 | HOXA-4 | homeobox A4 | 3201 | CCATCTGGACCATAATAGGGACACC (SEQ ID NO: 1565) |
| | | | | | CGGTTAAGAGTCTGTGCTGGTTGCC (SEQ ID NO: 1566) |
| | | | | | GAGAACAGTTGTCCAGGATGCTGGC (SEQ ID NO: 1567) |
| | | | | | ACAACCTGCTGGCCCGAAACAAGGC (SEQ ID NO: 1568) |
| | | | | | AACAAGGCTGCCAGGTGTGGAACAG (SEQ ID NO: 1569) |
| | | | | | AACAGCCCATGCTGACTGGGGACAT (SEQ ID NO: 1570) |
| | | | | | GACTGGGGACATACACTTGCATCTT (SEQ ID NO: 1571) |
| | | | | | CCCCATTAAGGCAGCTCATCCAAGC (SEQ ID NO: 1572) |
| | | | | | GACATGTTAGTTTTTCTTACTTGCA (SEQ ID NO: 1573) |
| | | | | | CAGAGATACTGTTGTCACCTTCTGG (SEQ ID NO: 1574) |
| | | | | | GGGGAGTGCATTTAGACTTATGTGG (SEQ ID NO: 1575) |
| 204778_x_at | HOXB7 | HHO.C1, HOX2, | homeobox B7 | 3217 | CCGGCTATGGGCTCGAGCCGAGTTC (SEQ ID NO: 1576) |
| | | HOX2C, Hox-2.3 | | | GGGCGCCAAGGAGCAGAGGGACTCG (SEQ ID NO: 1577) |
| | | | | | GGACTCGGACTTGGCGGCCGAGAGT (SEQ ID NO: 1578) |
| | | | | | GGCCGAGAGTAACTTCCGGATCTAC (SEQ ID NO: 1579) |
| | | | | | TCTACCCCTGGATGCGAAGCTCAGG (SEQ ID NO: 1580) |
| | | | | | CAGGAACTGACCGCAAACGAGGCCG (SEQ ID NO: 1581) |
| | | | | | CGCTACCAGACCCTGGAGCTGGAGA (SEQ ID NO: 1582) |
| | | | | | AGAATTTCACTACAATCGCTACCTG (SEQ ID NO: 1583) |
| | | | | | ACGCTCTGCCTCACGGAAAGACAGA (SEQ ID NO: 1584) |
| | | | | | ACCACCGGCCAAGACAGGGCTGAAG (SEQ ID NO: 1585) |
| | | | | | GCCCAGCTCTGGGAACTGAATCAGG (SEQ ID NO: 1586) |
| 205580_s_at | HRH1 | H1-R, hisH1 | histamine receptor | 3269 | TTAGGTGATAGCCAATCCTTCTCTC (SEQ ID NO: 1587) |
| | | H1 | | | TCGAACGGACTCAGATACCACCACA (SEQ ID NO: 1588) |
| | | | | | GAGGAGTGGGTCTAACACAGGCCTG (SEQ ID NO: 1589) |
| | | | | | TTTACTTGGAAGAGGCTCCGCTCGC (SEQ ID NO: 1590) |
| | | | | | CTCCGCTCGCATTCAAGACAGTATG (SEQ ID NO: 1591) |
| | | | | | GGTTTTATCATGGCAGCCTTCATCC (SEQ ID NO: 1592) |
| | | | | | GGTCATTGCCTTCTGCAAGAACTGT (SEQ ID NO: 1593) |
| | | | | | CATCTACCCCTTGTGCAATGAGAAC (SEQ ID NO: 1594) |
| | | | | | GAGAATTCTGCATATTCGCTCCTAA (SEQ ID NO: 1595) |
| | | | | | CCAGGCAGGCACCTGGGCTTTCTGG (SEQ ID NO: 1596) |
| | | | | | GAATCCAAACCACAGTCTTAGGGGC (SEQ ID NO: 1597) |
| 208744_x_at | HSPH1 | RP11-173P16.1, | heat shock | 10808 | GAAGGGGGCCTTTTGAGCTAGAAGC (SEQ ID NO: 1598) |
| | | DKFZp686M05240, | 105 kDa/110 kDa | | TAGAAGCTTTCTATTCTGATCCCCA (SEQ ID NO: 1599) |
| | | HSP105, | protein 1 | | GATCCCCAAGGAGTTCCATATCCAG (SEQ ID NO: 1600) |
| | | HSP105A, | | | AATAGGCCGCTTTGTAGTTCAGAAT (SEQ ID NO: 1601) |
| | | HSP105B, | | | GTCAACACCCATGGCATTTTCACCA (SEQ ID NO: 1602) |
| | | KIAA0201, NY- | | | GTGAAGCTGGAACACAGCCCCAGGT (SEQ ID NO: 1603) |
| | | CO-25 | | | AGCCCCAGGTACAAACTGATGCTCA (SEQ ID NO: 1604) |
| | | | | | TGATGCTCAACAAACCTCACAGTCT (SEQ ID NO: 1605) |
| | | | | | CCCTTCACCTGAACTTACCTCAGAA (SEQ ID NO: 1606) |
| | | | | | AAAGTTGACCAGCCTCCAGAAGCTA (SEQ ID NO: 1607) |
| | | | | | GAGCTGCCTATTGAAGCCAACTTGG (SEQ ID NO: 1608) |
| 216615_s_at | HTR3A | 5-HT-3, 5-HT3A, | 5-hydroxytryptamine | 3359 | CCCCTCAGGACCCAGGGAATGCCAG (SEQ ID NO: 1609) |
| | | 5-HT3R, 5HT3R, | (serotonin) receptor | | GCCAGGGACATTTTCAAGACACAGA (SEQ ID NO: 1610) |
| | | HTR3 | 3A | | CCCTGTTTCCAATGCCAATTCATCT (SEQ ID NO: 1611) |
| | | | | | ATGCCAATTCATCTCAGCAATCACA (SEQ ID NO: 1612) |
| | | | | | CACAAGCCAAGGTCTGAACCCTTCC (SEQ ID NO: 1613) |
| | | | | | TGGGTGTTCAAGGCCCTTACACCCT (SEQ ID NO: 1614) |
| | | | | | CCAGCAGCTCACCATGGCTTTAAAA (SEQ ID NO: 1615) |
| | | | | | GGCTTTAAAACATGCTCTCTTAGAT (SEQ ID NO: 1616) |
| | | | | | TCAGGAGAAACTCGGGCACTCCCTA (SEQ ID NO: 1617) |
| | | | | | ACTCCCTAAGTCCACTCTAGTTGTG (SEQ ID NO: 1618) |
| | | | | | TAGTTGTGGACTTTTCCCCATTGAC (SEQ ID NO: 1619) |
| 217756_x_at | HYPK /// | /// 4F5REL, | Huntingtin interacting | 25764 | GCAACGTCCGACAGAACGAGGGGAC (SEQ ID NO: 1620) |
| | SERF2 | FAM2C, | protein K /// small | 10169 | GAGGGGACGTAACGGAGGCAGGTTG (SEQ ID NO: 1621) |
| | | FLJ20431, | EDRK-rich factor 2 | | CGGAGGCAGGTTGGAGCCGCTGCCG (SEQ ID NO: 1622) |
| | | FLJ37527, | | | GCTCGCCCGCCAGAAGAATATGAAA (SEQ ID NO: 1623) |
| | | FLJ38557, | | | AAGCGCCGAGATGACGGGCTTTCTG (SEQ ID NO: 1624) |
| | | H4F5rel, | | | CCGCCCGCAAGCAGAGGGACTCGGA (SEQ ID NO: 1625) |
| | | HsT17089, | | | GGGACTCGGAGATCATGCAGCAGAA (SEQ ID NO: 1626) |
| | | MGC48826 | | | GGAACCCAAGTAGCTTTGTGGCTTC (SEQ ID NO: 1627) |
| | | | | | TCCTCCTGTAGTGCTCACAGGTCCC (SEQ ID NO: 1628) |
| | | | | | CTTCCCAGTGTTTTTTATTCCTGTG (SEQ ID NO: 1629) |
| | | | | | TTATTCCTGTGGGGCTCACCCCAAA (SEQ ID NO: 1630) |
| 215485_s_at | ICAM1 | BB2, CD54, | intercellular adhesion | 3383 | ACAGTGAAGTGTGAGGCCCACCCTA (SEQ ID NO: 1631) |
| | | P3.58 | molecule 1 (CD54), | | GCCCACCCTAGAGCCAAGGTGACGC (SEQ ID NO: 1632) |
| | | | human rhinovirus | | GTGACGCTGAATGGGGTTCCAGCCC (SEQ ID NO: 1633) |
| | | | receptor | | TATACACAAGAACCAGACCCGGGAG (SEQ ID NO: 1634) |
| | | | | | GTCCGGGAAACTGGACGTGGCCAGA (SEQ ID NO: 1635) |
| | | | | | CATTGCCCGAGCTCAAGTGTCTAAA (SEQ ID NO: 1636) |
| | | | | | TAAAGGATGGCACTTTCCCACTGCC (SEQ ID NO: 1637) |
| | | | | | AGTGACTGTCACTCGAGATCTTGAG (SEQ ID NO: 1638) |
| | | | | | CGAGATCTTGAGGGCACCTACCTCT (SEQ ID NO: 1639) |
| | | | | | GAGCACTCAAGGGGAGGTCACCCGC (SEQ ID NO: 1640) |
| | | | | | CCCCCCGGTATGAGATTGTCATCAT (SEQ ID NO: 1641) |
| 203327_at | IDE | RP11-366I13.1, | insulin-degrading | 3416 | TCATCACTTCCTTCTGGTTTTATGT (SEQ ID NO: 1642) |
| | | FLJ35968, | enzyme | | TTCCGATCCACCACTGTACTAGTAA (SEQ ID NO: 1643) |
| | | INSULYSIN | | | GTCTTCCATGAACCTGCAATTGTTT (SEQ ID NO: 1644) |
| | | | | | TATGCGTCAGCATTTTCCAATTTCC (SEQ ID NO: 1645) |
| | | | | | AGGTTGGATCTAGAGCTGCTGTTGA (SEQ ID NO: 1646) |
| | | | | | GCTGCTGTTGATCACTCAGGCATAC (SEQ ID NO: 1647) |
| | | | | | GTCCAAGCTGCAGTCCATGAGCAAT (SEQ ID NO: 1648) |
| | | | | | CATGAGCAATAACAGACTACCCCAG (SEQ ID NO: 1649) |
| | | | | | GACTACCCCAGATACTGCAGTTTAC (SEQ ID NO: 1650) |
| | | | | | GAAAGTCTTCTTTTTTGACCAGTTG (SEQ ID NO: 1651) |
| | | | | | GAGGGAGCATGTGACACAGCCAGTA (SEQ ID NO: 1652) |
| 204703_at | IFT88 | AW552028, | intraflagellar transport | 8100 | AATGTCTGCGTTTCTTAGTTCGTCT (SEQ ID NO: 1653) |
| | | Tg737, | 88 homolog | | AGTTCGTCTCTGCACAGATCTTGGA (SEQ ID NO: 1654) |
| | | Tg737Rpw, | (Chlamydomonas) | | GGGAACAGCGCATAAAGTCAGGCAG (SEQ ID NO: 1655) |
| | | TgN737Rpw, | | | GGGCTCCCGTGGCAAAAGAGAAGGA (SEQ ID NO: 1656) |
| | | Ttc10, flexo, fxo, | | | GTGCTAGCGGTGATAGTGGCCAGAA (SEQ ID NO: 1657) |
| | | orpk, polaris | | | GGTGAACGACTAAGTGCCAGACTCA (SEQ ID NO: 1658) |
| | | | | | AAGTGCCAGACTCAGAGCTTTACCT (SEQ ID NO: 1659) |
| | | | | | AAATAGATGCCTCCTATGTGGACCC (SEQ ID NO: 1660) |
| | | | | | CCCACTTGGCCCTCAAATAGAACGA (SEQ ID NO: 1661) |
| | | | | | GAAATTGCCTTATGAGATCATCCTC (SEQ ID NO: 1662) |
| | | | | | AGATCATCCTCATGTTAAACCTTGG (SEQ ID NO: 1663) |
| 207375_s_at | IL15RA | RP11-536K7.2, | interleukin 15 | 3601 | TGCGCGAGGGAGCAGGTTCTCCGCA (SEQ ID NO: 1664) |
| | | MGC104179 | receptor, alpha | | CCCAGCTTCCCAGGAGAGACCAAAG (SEQ ID NO: 1665) |
| | | | | | GAGACCAAAGGCTTCTGAGCAGGAT (SEQ ID NO: 1666) |
| | | | | | GTGAGCTGCCTGGAATACATGTGGT (SEQ ID NO: 1667) |
| | | | | | CATACCCAAAGCTCTCTGTCAATTA (SEQ ID NO: 1668) |
| | | | | | GCTTCTCCTGGCGTGGGAGACGTCT (SEQ ID NO: 1669) |
| | | | | | AGACACCAGCGTTTGGGCTTCTAAC (SEQ ID NO: 1670) |
| | | | | | GCTGCTCTGCACACATGGACAGGGA (SEQ ID NO: 1671) |
| | | | | | GAGATGCTGAGCCCAGCGAATCCTC (SEQ ID NO: 1672) |
| | | | | | CCAGCGAATCCTCTCCATTGAAGGA (SEQ ID NO: 1673) |
| | | | | | ACTCAACTCAGTGCCATTTTACGAA (SEQ ID NO: 1674) |
| 207526_s_at | IL1RL1 | DER4, FIT-1, | interleukin 1 receptor- | 9173 | GTGTTTCTGAGACTTTGATCACCTG (SEQ ID NO: 1675) |
| | | MGC32623, ST2, | like 1 | | TGATCACCTGAACTTTCTCTAGCAA (SEQ ID NO: 1676) |
| | | ST2L, ST2V, T1 | | | GGAGTGTGGTTCCAAGAGATCCATC (SEQ ID NO: 1677) |
| | | | | | GCCTGTGCCATAAAATGTGCTTCTC (SEQ ID NO: 1678) |
| | | | | | TCTTCGGGATGTTGTTTGCTGTCTG (SEQ ID NO: 1679) |
| | | | | | GATCTTTGTAGACTGTTCCTGTTTG (SEQ ID NO: 1680) |
| | | | | | AGACTGTTCCTGTTTGCTGGGAGCT (SEQ ID NO: 1681) |
| | | | | | CTCTGCTGCTTAAATTGTTCGTCCT (SEQ ID NO: 1682) |
| | | | | | CCTCCTATCGTTGGTTTGTCTAGAA (SEQ ID NO: 1683) |
| | | | | | GTCTAGAACACTCAGCTGCTTCTTT (SEQ ID NO: 1684) |
| | | | | | CTAACTTTATGAACTCCCTCTGTGT (SEQ ID NO: 1685) |
| 200955_at | IMMT | DKFZp779P1653, | inner membrane | 10989 | AGAGCTCTGCCCTGAGGATATAAAC (SEQ ID NO: 1686) |
| | | HMP, | protein, mitochondrial | | ATTACTGTCATATGCTTCCTATTGC (SEQ ID NO: 1687) |
| | | MGC111146, | (mitofilin) | | GGGGGAATCCAGACGAGTGGCACAG (SEQ ID NO: 1688) |
| | | P87, P87/89, | | | GAAGCCCGAATGACCCTAGAAACGA (SEQ ID NO: 1689) |
| | | P89, PIG4, | | | AATCCTGACAGCATATGCCAGCGCC (SEQ ID NO: 1690) |
| | | PIG52 | | | GCCAGCGCCGTAGGAATAGGAACCA (SEQ ID NO: 1691) |
| | | | | | ATAGGAACCACTCAGGTGCAGCCAG (SEQ ID NO: 1692) |
| | | | | | GCAGCGAGAGTCCCGGACTTGTCTA (SEQ ID NO: 1693) |
| | | | | | TATTCTTTCCATTTGCTATCATGTC (SEQ ID NO: 1694) |
| | | | | | GAACGCCAGGAGTGCTTTCTTTGCA (SEQ ID NO: 1695) |
| | | | | | GATCTCTACTACTGTTGATTTTGCC (SEQ ID NO: 1696) |
| 33304_at | ISG20 | CD25, HEM45 | interferon stimulated | 3669 | GTTCATCCGGCCTGAGGGAGAGATC (SEQ ID NO: 1697) |
| | | | exonuclease gene | | TCATCCGGCCTGAGGGAGAGATCAC (SEQ ID NO: 1698) |
| | | | 20 kDa | | CCGGCCTGAGGGAGAGATCACCGAT (SEQ ID NO: 1699) |
| | | | | | CCAGGCTAGAGATCCTGCAGCTCCT (SEQ ID NO: 1700) |
| | | | | | TAGAGATCCTGCAGCTCCTGAAAGG (SEQ ID NO: 1701) |
| | | | | | TGGTGGGTCATGACCTGAAGCACGA (SEQ ID NO: 1702) |
| | | | | | ACATGAGCGGCTACACAATCTACGA (SEQ ID NO: 1703) |
| | | | | | CATGAGCGGCTACACAATCTACGAC (SEQ ID NO: 1704) |
| | | | | | AAGCTGGACCACTGCAGGCGTGTCT (SEQ ID NO: 1705) |
| | | | | | AGAGCATCCAGAACAGCCTGCTTGG (SEQ ID NO: 1706) |
| | | | | | CCCGCCTGGCTGTGTCAGACTGAAG (SEQ ID NO: 1707) |
| | | | | | CCGCCTGGCTGTGTCAGACTGAAGC (SEQ ID NO: 1708) |
| | | | | | GGCTGTGTCAGACTGAAGCCCCATC (SEQ ID NO: 1709) |
| | | | | | GAGGCTTTCGGCTTTTTGGGACAGC (SEQ ID NO: 1710) |
| | | | | | GGCTTTTTGGGACAGCAACTACCTT (SEQ ID NO: 1711) |
| | | | | | GGACAGCAACTACCTTGCTTTTGGA (SEQ ID NO: 1712) |
| 205055_at | ITGAE | CD103, | integrin, alpha E | 3682 | AGAGATCTCCTGGGATCACTCTGAG (SEQ ID NO: 1713) |
| | | HUMINAE, | (antigen CD103, | | TCACTGTCGTCTTCCTGAAAGATGA (SEQ ID NO: 1714) |
| | | MGC141996 | human mucosal | | TACCATTCTTTGCCTATCATCATTA (SEQ ID NO: 1715) |
| | | | lymphocyte antigen | | AAGGCAGCGTTGGTGGACTTCTGGT (SEQ ID NO: 1716) |
| | | | 1; alpha polypeptide) | | GGACTTCTGGTGTTGATCGTGATTC (SEQ ID NO: 1717) |
| | | | | | TGATCGTGATTCTGGTCATCCTGTT (SEQ ID NO: 1718) |
| | | | | | GTCATCCTGTTCAAGTGTGGCTTTT (SEQ ID NO: 1719) |
| | | | | | CTGGGAGAGGCTATCAGCCAGTCCT (SEQ ID NO: 1720) |
| | | | | | CTTTGCATTACTTTTTCCTTCAGGA (SEQ ID NO: 1721) |
| | | | | | TAGTTTTTAACCATACATTGTCCCA (SEQ ID NO: 1722) |
| | | | | | CATTGTCCCAAAAGTGTCTGTGCAT (SEQ ID NO: 1723) |
| 204487_s_at | KCNQ1 | GAIP, RGSGAIP | potassium voltage- | 3784 | GCCCAGGAGCCCATTTGGAGGGCCT (SEQ ID NO: 1724) |
| | | | gated channel, KQT- | | AGACTGTGGAGACTGCTCCTGAGCC (SEQ ID NO: 1725) |
| | | | like subfamily, | | CATTTCCCCAGGGCACGTGGTTGAG (SEQ ID NO: 1726) |
| | | | member 1 | | GCACGTGGTTGAGTGGGGGGAACGC (SEQ ID NO: 1727) |
| | | | | | ACTTCCCTGGGTTAGACTGCCAGCT (SEQ ID NO: 1728) |
| | | | | | GCCAGCTGCTGAGCCGCAGAGAAGT (SEQ ID NO: 1729) |
| | | | | | GCCGCAGAGAAGTGACGGTTCCTAC (SEQ ID NO: 1730) |
| | | | | | GACGGTTCCTACACAGGACAGGGGT (SEQ ID NO: 1731) |
| | | | | | TTCCTTCTGGGCATTACATCGCATA (SEQ ID NO: 1732) |
| | | | | | GGGCATTACATCGCATAGAAATCAA (SEQ ID NO: 1733) |
| | | | | | TAATTTGTGGTGATTTGGATCTGTG (SEQ ID NO: 1734) |
| 212357_at | KIAA0280 | KIAA0280, | KIAA0280 | 23201 | GATAGCAGGGGGATTTTCCTTCCTA (SEQ ID NO: 1735) |
| | | TCRP1 | | | GCAAAGTACCGGCCCATAGGAATCA (SEQ ID NO: 1736) |
| | | | | | CCTTTCTCCTCAGGCCTAAGAGAGT (SEQ ID NO: 1737) |
| | | | | | AGAGAGGAGACTGTCCCAGAGACCT (SEQ ID NO: 1738) |
| | | | | | TCCTCGGGTCAGCCAGATAGTCTGG (SEQ ID NO: 1739) |
| | | | | | GGGGTAAGGCCAGGCCTCTAGCTAC (SEQ ID NO: 1740) |
| | | | | | GGCCTCTAGCTACTTGGAGTTGTCT (SEQ ID NO: 1741) |
| | | | | | TCCCTGTTTCATATCACATGACAGA (SEQ ID NO: 1742) |
| | | | | | GACAGAGAAACCTGTTCTCATGGCA (SEQ ID NO: 1743) |
| | | | | | TCTCATGGCATGTAACATCCCTGTG (SEQ ID NO: 1744) |
| | | | | | CATCCCTGTGAAGAGAGCGTTGTAT (SEQ ID NO: 1745) |
| 212311_at | KIAA0746 /// | DKFZp781J1697, | KIAA0746 protein /// | 23231 | GGAAGGTACGATAATCCCACACCAT (SEQ ID NO: 1746) |
| | SERINC2 | FLJ21629, | serine incorporator 2 | 347735 | TCCCACACCATATCTTGGATTTCTT (SEQ ID NO: 1747) |
| | | FLJ41299 /// | | | TTGACTCAACTCTCCATTCTAATAA (SEQ ID NO: 1748) |
| | | TDE2; TDE2L; | | | GAGCCACAGTAACGAGGAGTCCTTC (SEQ ID NO: 1749) |
| | | FKSG84; | | | CTCAGCCCTGATCTACTTTCTGGGA (SEQ ID NO: 1750) |
| | | PRO0899; | | | TTTCTGGGAACCTTTCTGCTATCCA (SEQ ID NO: 1751) |
| | | MGC90340 | | | GCAGTATTTCCAGTCTGTCTCAGCA (SEQ ID NO: 1752) |
| | | | | | TGCAGATGCCTCTGACCAAGACCAG (SEQ ID NO: 1753) |
| | | | | | AAGACCAGCCCACAGTAACTAATAA (SEQ ID NO: 1754) |
| | | | | | GTAACTAATAACCCGGAGCCACGTG (SEQ ID NO: 1755) |
| | | | | | CACGTGGGTGAACTGTGCACTCCAG (SEQ ID NO: 1756) |
| 212314_at | KIAA0746 /// | DKFZp781J1697, | KIAA0746 protein /// | 23231 | TATTTTGGTACCTGTGCTTGCCACA (SEQ ID NO: 1757) |
| | SERINC2 | FLJ21629, | serine incorporator 2 | 347735 | TTGATAGATTTCTCTTTGACTTCCA (SEQ ID NO: 1758) |
| | | FLJ41299 /// | | | TTGACTTCCAAGACCTAGCAGTTAT (SEQ ID NO: 1759) |
| | | TDE2; TDE2L; | | | GTCCTAGTGCTTCCGAATCATTTAA (SEQ ID NO: 1760) |
| | | FKSG84; | | | AATGGCATTGTCGGATATCTTTTAC (SEQ ID NO: 1761) |
| | | PRO0899; | | | ATCTTTTACATTTCAATTGCAATCC (SEQ ID NO: 1762) |
| | | MGC90340 | | | AGTACTTAACTGTAGTCTTCTCCAT (SEQ ID NO: 1763) |
| | | | | | GTAGTCTTCTCCATGAATTACACGT (SEQ ID NO: 1764) |
| | | | | | GCCTCTAGCTTATAGTTTCATCCCT (SEQ ID NO: 1765) |
| | | | | | GCCTGCGTGAGTCTGTACAGGGATA (SEQ ID NO: 1766) |
| | | | | | GGTCCAAACTACTCTTTGCACTACT (SEQ ID NO: 1767) |
| 202962_at | KIF13B | GAKIN, | kinesin family | 23303 | ATGTTTCTGGGCCTACCAAGGTTCA (SEQ ID NO: 1768) |
| | | KIAA0639 | member 13B | | CTGGGAATGTGACTGCTGGAGCCTG (SEQ ID NO: 1769) |
| | | | | | TGAGAGTGTGTGTTGGTGGCTGTGC (SEQ ID NO: 1770) |
| | | | | | ACAAGACGGGAGTCACCCTGTGCTT (SEQ ID NO: 1771) |
| | | | | | CCAAGATACTGACCCATTGAACCCC (SEQ ID NO: 1772) |
| | | | | | TTCTCTCCACAAAGTCCGTGGTGCC (SEQ ID NO: 1773) |
| | | | | | GGTGGGCTGCAGACACTAATGGTGT (SEQ ID NO: 1774) |
| | | | | | GGTCTTGGAACAGCTTCTCTATGTG (SEQ ID NO: 1775) |
| | | | | | GCTTCTCTATGTGTGGATTCGTGTA (SEQ ID NO: 1776) |
| | | | | | AACCACCTGTAATTTTGTACCATAT (SEQ ID NO: 1777) |
| | | | | | GTACCATATGTGTCTTTCCATCAAA (SEQ ID NO: 1778) |
| 213656_s_at | KLC1 | KLC, KNS2, | kinesin light chain 1 | 3831 | CAGCAGCAGTGGCCTGGAAGACGCC (SEQ ID NO: 1779) |
| | | KNS2A, | | | GCAGTGGCCTGGAAGACGCCACCGC (SEQ ID NO: 1780) |
| | | MGC15245 | | | TTCTCCTGCGTCTGTGTGCATAGGA (SEQ ID NO: 1781) |
| | | | | | CCTGCGTCTGTGTGCATAGGACATG (SEQ ID NO: 1782) |
| | | | | | GTCTGTGTGCATAGGACATGATACT (SEQ ID NO: 1783) |
| | | | | | GACATGATACTAATAACCACACGGC (SEQ ID NO: 1784) |
| | | | | | AATAACCACACGGCTGGCGTGACCT (SEQ ID NO: 1785) |
| | | | | | CCATGTGTAACTTCCTCACGTTGTG (SEQ ID NO: 1786) |
| | | | | | TGTAACTTCCTCACGTTGTGTGCGA (SEQ ID NO: 1787) |
| | | | | | ACTTCCTCACGTTGTGTGCGATAAC (SEQ ID NO: 1788) |
| | | | | | GTGTGCGATAACGTATTTTATTGTA (SEQ ID NO: 1789) |
| 206966_s_at | KLF12 | AP-2rep, | Kruppel-like factor 12 | 11278 | CCGCCTTCAAGTCCCATGAATTTAC (SEQ ID NO: 1790) |
| | | AP2REP, | | | CAAACTGAGTCATGTTCACCGCATC (SEQ ID NO: 1791) |
| | | HSPC122 | | | CATCCCCGTGGTGGTACAGTCGGTG (SEQ ID NO: 1792) |
| | | | | | TGGTACAGTCGGTGCCTGTTGTCTA (SEQ ID NO: 1793) |
| | | | | | GTCTACACAGCTGTAAGGTCACCTG (SEQ ID NO: 1794) |
| | | | | | GAACAACACTATTGTCGTGCCGCTT (SEQ ID NO: 1795) |
| | | | | | GTCGTGCCGCTTTTGGAGGATGGGA (SEQ ID NO: 1796) |
| | | | | | TGGCAAAGCACAAATGGACCCCCGA (SEQ ID NO: 1797) |
| | | | | | AGGCCTATCTCCCAGACAAAGTAAA (SEQ ID NO: 1798) |
| | | | | | AAATGTGACCTTAGATAGCGTTAAT (SEQ ID NO: 1799) |
| | | | | | CCCTTTCCATAGCCAGAGCAGTACA (SEQ ID NO: 1800) |
| 205569_at | LAMP3 | CD208, DC- | lysosomal-associated | 27074 | AGCCTATCCTTAATAAATCCTCCAC (SEQ ID NO: 1801) |
| | | LAMP, DCLAMP, | membrane protein 3 | | AATCCTCCACTCTCTGGAAGGAGAC (SEQ ID NO: 1802) |
| | | LAMP, TSC403 | | | GAAACTTCGCTAACCAACTGTTCTT (SEQ ID NO: 1803) |
| | | | | | GTTCTTTCTTGAGTGTATAGCCCCA (SEQ ID NO: 1804) |
| | | | | | ATAGCCCCATCTTGTGGTAACTTGC (SEQ ID NO: 1805) |
| | | | | | GCTTCTGCACTTCATATCCATATTT (SEQ ID NO: 1806) |
| | | | | | ATCCATATTTCCTATTGTTCACTTT (SEQ ID NO: 1807) |
| | | | | | TTTATTCTGTAGAGCAGCCTGCCAA (SEQ ID NO: 1808) |
| | | | | | AAAGTCCACATAACCCTAGAATTCT (SEQ ID NO: 1809) |
| | | | | | GCCTAGAGACCATGTTGACTTTCCT (SEQ ID NO: 1810) |
| | | | | | GGCAAGGTCCTGACTTTAGTCTTAA (SEQ ID NO: 1811) |
| 202019_s_at | LANCL1 | GPR69A, p40 | LanC lantibiotic | 10314 | TCTACAACCTCACACAGGACATGAA (SEQ ID NO: 1812) |
| | | | synthetase | | GAAGTACCTGTATAGGGCCTGTAAG (SEQ ID NO: 1813) |
| | | | component C-like 1 | | GGGCCTGTAAGTTTGCTGAATGGTG (SEQ ID NO: 1814) |
| | | | (bacterial) | | TTCTCTCTCTTTGAAGGAATGGCTG (SEQ ID NO: 1815) |
| | | | | | CAATATATTTCCTGGCTGACCTGCT (SEQ ID NO: 1816) |
| | | | | | CCTGCTAGTCCCCACAAAAGCCAGG (SEQ ID NO: 1817) |
| | | | | | AGCCAGGTTCCCTGCATTTGAACTC (SEQ ID NO: 1818) |
| | | | | | AAAGGATAGCATGCCACCTGCAACT (SEQ ID NO: 1819) |
| | | | | | CTGCATGACCCTTTCTGTATATTCA (SEQ ID NO: 1820) |
| | | | | | AACCCAAGCTAAGTGCTTCCGTTGC (SEQ ID NO: 1821) |
| | | | | | GTGCTTCCGTTGCTTTCCAAGGAAA (SEQ ID NO: 1822) |
| 217933_s_at | LAP3 | LAP, LAPEP, | leucine | 51056 | ATGTACAGCTGCAGCATTCCTGAAA (SEQ ID NO: 1823) |
| | | PEPS | aminopeptidase 3 | | GTAACTCATCCTAAGTGGGCACATT (SEQ ID NO: 1824) |
| | | | | | TGGGCACATTTAGACATAGCAGGCG (SEQ ID NO: 1825) |
| | | | | | GCAGGCGTGATGACCAACAAAGATG (SEQ ID NO: 1826) |
| | | | | | GAAGTTCCCTATCTACGGAAAGGCA (SEQ ID NO: 1827) |
| | | | | | GAAAGGCATGACTGGGAGGCCCACA (SEQ ID NO: 1828) |
| | | | | | GGCCCACAAGGACTCTCATTGAGTT (SEQ ID NO: 1829) |
| | | | | | GAGTTCTTACTTCGTTTCAGTCAAG (SEQ ID NO: 1830) |
| | | | | | GACAATGCTTAGTTCAGATACTCAA (SEQ ID NO: 1831) |
| | | | | | AGAACTTCCTAATCACTTTTCAGAG (SEQ ID NO: 1832) |
| | | | | | GAAAATTACTATGCACTTGTCAGAA (SEQ ID NO: 1833) |
| 218717_s_at | LEPREL1 | FLJ10718, | leprecan-like 1 | 55214 | GAAACTACAACTCTCACGTGTGCTC (SEQ ID NO: 1834) |
| | | MLAT4, P3H2 | | | TAGGTGCTAACGGCCATGAGCTCCG (SEQ ID NO: 1835) |
| | | | | | GCTCCGACTCTCCAAAGTGAGCTCC (SEQ ID NO: 1836) |
| | | | | | CCACGCTGCCTCAGGTATCATGGGC (SEQ ID NO: 1837) |
| | | | | | TGGGCGTAATGATCACCCAGGCTCC (SEQ ID NO: 1838) |
| | | | | | CAGGCTCCGGGAGATCTCATGGATG (SEQ ID NO: 1839) |
| | | | | | GAATTTTTGGCTCTGGTGTTTTCGC (SEQ ID NO: 1840) |
| | | | | | ACTGGAAGCTTTGATTCACCCTCCA (SEQ ID NO: 1841) |
| | | | | | TAGTAGAATGGCTTTTCACCCTGGT (SEQ ID NO: 1842) |
| | | | | | GAAACTCTTGAATACTGCCTGCATC (SEQ ID NO: 1843) |
| | | | | | CACCATGGCCAGGTTGCCTAGGAGT (SEQ ID NO: 1844) |
| 203236_s_at | LGALS9 | HUAT, | lectin, galactoside- | 3965 | ATCCAGCTGACCCATGTGCAGACAT (SEQ ID NO: 1845) |
| | | LGALS9A, | binding, soluble, 9 | | ATGTGCAGACATAGGCGGCTTCCTG (SEQ ID NO: 1846) |
| | | MGC117375, | (galectin 9) | | GGATCTGGGCTTTAATGCAGAGGCC (SEQ ID NO: 1847) |
| | | MGC125973, | | | TTTAATGCAGAGGCCATGTCCTTGT (SEQ ID NO: 1848) |
| | | MGC125974 | | | GCCACCCTGGAACGGAGAAGGCAGC (SEQ ID NO: 1849) |
| | | | | | AGGCAGCTGACGGGGATTGCCTTCC (SEQ ID NO: 1850) |
| | | | | | CCCAGGAGGCAGGCACAGCCAGGGA (SEQ ID NO: 1851) |
| | | | | | GGAGAGGGGAGGAGTGGGCAGTGAA (SEQ ID NO: 1852) |
| | | | | | CCCCTCCTGGAAAGCAGGCCTGATG (SEQ ID NO: 1853) |
| | | | | | TGGAAAGCAGGCCTGATGGCTTCCC (SEQ ID NO: 1854) |
| | | | | | CCACCTGACCAGAGTGTTCTCTTCA (SEQ ID NO: 1855) |
| 218272_at | LL22NC03- | LL22NC03- | hypothetical protein | 55020 | TACAGATCAGCGCGCACATGGGGTC (SEQ ID NO: 1856) |
| | 5H6.5 | 5H6.5, FLJ20699 | FLJ20699 | | TGTCTCTGGGGCGAGTCTTGGAGCC (SEQ ID NO: 1857) |
| | | | | | CTGTTGGTGCTGATTTGGACCCGTT (SEQ ID NO: 1858) |
| | | | | | GAGCTGCTCTGTTGTGGCAAAACCC (SEQ ID NO: 1859) |
| | | | | | AAGTGCTGTGATGTGTGGCTGTGAC (SEQ ID NO: 1860) |
| | | | | | GGCTGTGACAAGCCTGGAGCGGGTC (SEQ ID NO: 1861) |
| | | | | | CACTGAACGCCTGCTGAATGTGCCG (SEQ ID NO: 1862) |
| | | | | | CTGAATGTGCCGCTGACTCAGCTGT (SEQ ID NO: 1863) |
| | | | | | GAAGCAGGCAGGCCTCTCAGAAGGG (SEQ ID NO: 1864) |
| | | | | | GAGGCCTCCAAATCTATTGAGTCCC (SEQ ID NO: 1865) |
| | | | | | TTGAGTCCCCACAGTTTGCTCAAGC (SEQ ID NO: 1866) |
| 219095_at | LOC100131005 | /// JMJD7, | similar to PLA2G4B | 100131005 | TGGGGAGGTGAACCTGTCTTCATCG (SEQ ID NO: 1867) |
| | /// | FLJ20543, | protein /// jumonji | 100137047 | TCTCCCTACCACTACACGAAGGTGA (SEQ ID NO: 1868) |
| | LOC100137047 | FLJ20656, | domain containing 7 | 8681 | GTGACCTACAGCCAGGAGGACGTGG (SEQ ID NO: 1869) |
| | /// | FLJ20807, | /// JMJD7-PLA2G4B | 100137049 | CTGCTGCACCTGACACATTACAATG (SEQ ID NO: 1870) |
| | LOC100137047- | FLJ77014, | readthrough | | GCGCCAGGCAGTGCAGCGGAGGCGG (SEQ ID NO: 1871) |
| | PLA2G4B /// | FLJ78330 /// | transcript /// | | TCCCTGGCTGCTGAGTTGCAGGTGG (SEQ ID NO: 1872) |
| | PLA2G4B | JMJD7- | phospholipase A2, | | GGAACTGTCATCACGCAGTGCTTCA (SEQ ID NO: 1873) |
| | | PLA2G4B, | group IVB (cytosolic) | | GCTTCAGAGCCTCGGGCTCAGGTGG (SEQ ID NO: 1874) |
| | | HsT16992, | | | TCCCAGGGTCCAGGCTGAGGGCTGG (SEQ ID NO: 1875) |
| | | PLA2G4B, | | | GGAGGCCAAGCCCATTTGTGTAATC (SEQ ID NO: 1876) |
| | | cPLA2-beta /// | | | CCTTCTGCGCTACCTTGAGTAGTTG (SEQ ID NO: 1877) |
| | | DKFZp686N12131, | | | |
| | | FLJ17693, | | | |
| | | FLJ42498, | | | |
| | | HsT16992, | | | |
| | | cPLA2-beta | | | |
| 201569_s_at | LOC100131861 | OMP85; SAM50; | sorting and assembly | 100131861 | GGATATGTGATGGCGTCCAGTTTGG (SEQ ID NO: 1878) |
| | /// | TOB55; TRG-3; | machinery | 25813 | GATGGCGTCCAGTTTGGAGCTGGGA (SEQ ID NO: 1879) |
| | SAMM50 | CGI-51; | component 50 | | GGCGTCCAGTTTGGAGCTGGGATAA (SEQ ID NO: 1880) |
| | | YNL026W | homolog (S. cerevisiae) | | GAGCTGGGATAAGGTTCCTGTAGCC (SEQ ID NO: 1881) |
| | | | /// | | ACACCGTCTCTCGAGGAAACGCGGT (SEQ ID NO: 1882) |
| | | | hypothetical protein | | GTCTCTCGAGGAAACGCGGTTCAGC (SEQ ID NO: 1883) |
| | | | LOC100131861 | | GAGGAAACGCGGTTCAGCGATTCTT (SEQ ID NO: 1884) |
| | | | | | GAAACGCGGTTCAGCGATTCTTTGA (SEQ ID NO: 1885) |
| | | | | | CGCGGTTCAGCGATTCTTTGACTGC (SEQ ID NO: 1886) |
| | | | | | GGTTCAGCGATTCTTTGACTGCGGA (SEQ ID NO: 1887) |
| | | | | | TTCAGCGATTCTTTGACTGCGGACC (SEQ ID NO: 1888) |
| 214133_at | LOC100133432 | /// mucin 6, | hypothetical protein | 100131861 | GACAGACAGGAAAACACCCACCAGC (SEQ ID NO: 1889) |
| | /// | gastric | LOC100131861 /// | 4588 | CTCCCTGGGGCACCGGTTGGAGAGG (SEQ ID NO: 1890) |
| | MUC6 | | mucin 6, oligomeric | | GAGGCGGACAGTGGTCACGGACCTC (SEQ ID NO: 1891) |
| | | | mucus/gel-forming | | GGCCCAGAACCCTGCAGATGAAGCC (SEQ ID NO: 1892) |
| | | | | | GACCCATCAGGGGCGAGGAGGGCAC (SEQ ID NO: 1893) |
| | | | | | GGAGGGCACGGAAACCTGTGCCGAG (SEQ ID NO: 1894) |
| | | | | | GTGCCGAGATGGGGGCAAGAGGCCC (SEQ ID NO: 1895) |
| | | | | | GAGGAGATCCCCAGAGTCAGGGAGG (SEQ ID NO: 1896) |
| | | | | | GCCTGTCCTTGGGAGAGGGCGTCAC (SEQ ID NO: 1897) |
| | | | | | TCACCCGGTCAGAGACTCCAAATAA (SEQ ID NO: 1898) |
| | | | | | GAGACTCCAAATAAACCGGTTCTTG (SEQ ID NO: 1899) |
| 220009_at | LONRF3 | RP23-145O4.1, | LON peptidase N- | 79836 | GGTTTCATTCGCTCAAATTATCCCT (SEQ ID NO: 1900) |
| | | 4932412G04Rik, | terminal domain and | | GATACTCAATCACTTTGGTCCCATG (SEQ ID NO: 1901) |
| | | 5730439E01Rik, | ring finger 3 | | AGAAAGACGCCGATCCTCAGATGAA (SEQ ID NO: 1902) |
| | | A830039N02Rik, | | | CTCAGATGAACCCGAATGGCCCAGC (SEQ ID NO: 1903) |
| | | AU023707, | | | GGTGGATGTTAGCAGTTCTTCCCTT (SEQ ID NO: 1904) |
| | | Rnf127 | | | GTTCTTCCCTTGGAAAGCCGAGCTC (SEQ ID NO: 1905) |
| | | | | | TCCCCTTCCTAGCAATGAGGTCCTT (SEQ ID NO: 1906) |
| | | | | | AATGGTATTCGACGAGTCCTGGCCT (SEQ ID NO: 1907) |
| | | | | | TGGCCTTCATATCCCGAAACCAAAA (SEQ ID NO: 1908) |
| | | | | | GTGGATTGCCGAAGAGGAGCTCCCA (SEQ ID NO: 1909) |
| | | | | | GAGGCATCAGCCTGCTGTTGATCAC (SEQ ID NO: 1910) |
| 220816_at | LPAR3 | EDG7, Edg-7, | lysophosphatidic acid | 23566 | TGGCCCCCATTTACAGCAGGAGTTA (SEQ ID NO: 1911) |
| | | FLJ98231, | receptor 3 | | GGAGTTACCTTGTTTTCTGGACAGT (SEQ ID NO: 1912) |
| | | GPCR, | | | CCTTCCTCATCATGGTTGTGGTGTA (SEQ ID NO: 1913) |
| | | HOFNH30, LP- | | | GTACCTGCGGATCTACGTGTACGTC (SEQ ID NO: 1914) |
| | | A3, LPA3, RP4- | | | GAGGAAAACCAACGTCTTGTCTCCG (SEQ ID NO: 1915) |
| | | 678I3 | | | TCTCCGCATACAAGTGGGTCCATCA (SEQ ID NO: 1916) |
| | | | | | GCCGGAGGACACCCATGAAGCTAAT (SEQ ID NO: 1917) |
| | | | | | GGGCGTTTGTGGTATGCTGGACCCC (SEQ ID NO: 1918) |
| | | | | | GAGAACCCAGAGAGGCGTCCCTCTC (SEQ ID NO: 1919) |
| | | | | | CAGGAGTGACACAGGCAGCCAGTAC (SEQ ID NO: 1920) |
| | | | | | GCACTTCCTAAACTCTGGATGCCTC (SEQ ID NO: 1921) |
| 201932_at | LRRC41 | MGC126571, | leucine rich repeat | 10489 | CTGGCTGGACCAGGATGCAGTCACA (SEQ ID NO: 1922) |
| | | MGC126573, | containing 41 | | GGCTACCTGCCATGTGGTTAGCGAC (SEQ ID NO: 1923) |
| | | MUF1, PP7759, | | | TGGTTAGCGACTCATGGACTCATCC (SEQ ID NO: 1924) |
| | | RP4-636H5.2 | | | TCCCAGGCCTTCGCAGATTATGTTA (SEQ ID NO: 1925) |
| | | | | | CGGTACCATCAGCTTGCAGGGGCTG (SEQ ID NO: 1926) |
| | | | | | TGCAGGGGCTGAAGCATGGGCTGCC (SEQ ID NO: 1927) |
| | | | | | GAGGCCCAGCAAACAGGCATTCTCA (SEQ ID NO: 1928) |
| | | | | | GGCATTCTCACAGCTGGGTTTATAG (SEQ ID NO: 1929) |
| | | | | | CTCAGTATCCTGGGAACCCTGGGCC (SEQ ID NO: 1930) |
| | | | | | ATAATTGCTGAAGAGATCCCCTCCC (SEQ ID NO: 1931) |
| | | | | | TCAAACCCTTTCATACCTTTATTCT (SEQ ID NO: 1932) |
| 212904_at | LRRC47 | KIAA1185; RP1- | leucine rich repeat | 57470 | TATGTATTCGTTGTTCTGTTTTTCC (SEQ ID NO: 1933) |
| | | 286D6.3 | containing 47 | | GATGATTCTGTCGTAAAGTGATGCT (SEQ ID NO: 1934) |
| | | | | | AAGTGATGCTATGTTGTCGTTACAA (SEQ ID NO: 1935) |
| | | | | | GTGCTGTTACGTGGCACTGTACAGT (SEQ ID NO: 1936) |
| | | | | | CAGTCATGTGCCACGTAACAGCGTC (SEQ ID NO: 1937) |
| | | | | | CACGTAACAGCGTCTGGGTCAGTGA (SEQ ID NO: 1938) |
| | | | | | GGGTCAGTGACGGACACTTACCTGA (SEQ ID NO: 1939) |
| | | | | | ACTTACCTGACAGCGGATCCACAAT (SEQ ID NO: 1940) |
| | | | | | GATCCACAATATTCTCGTGCAGTGT (SEQ ID NO: 1941) |
| | | | | | GCAGTGTGTTTGGAATCCTGGTCTG (SEQ ID NO: 1942) |
| | | | | | GTGAGTGATGTTCAGTCATGCTGCT (SEQ ID NO: 1943) |
| 211370_s_at | MAP2K5 | AI324775, | mitogen-activated | 5607 | CATTCTGATGTCTGGAGCTTAGGAA (SEQ ID NO: 1944) |
| | | AI428457, MEK5, | protein kinase kinase 5 | | CTCCAGCTTCTGCAGTGCATTGTTG (SEQ ID NO: 1945) |
| | | Mapkk5, Prkmk5 | | | TTGTTGATGAGGATTCGCCCGTCCT (SEQ ID NO: 1946) |
| | | | | | CCCGTCCTTCCAGTTGGAGAGTTCT (SEQ ID NO: 1947) |
| | | | | | GGAGAGTTCTCGGAGCCATTTGTAC (SEQ ID NO: 1948) |
| | | | | | GGAGCCATTTGTACATTTCATCACT (SEQ ID NO: 1949) |
| | | | | | ACATTTCATCACTCAGTGTATGCGA (SEQ ID NO: 1950) |
| | | | | | GCCAAAAGAAAGGCCAGCACCTGAA (SEQ ID NO: 1951) |
| | | | | | GCACCTGAAGAATTGATGGGCCACC (SEQ ID NO: 1952) |
| | | | | | AAGAATTGATGGGCCACCCGTTCAT (SEQ ID NO: 1953) |
| | | | | | ATGATGGAAATGCCGCCGTGGTGTC (SEQ ID NO: 1954) |
| 206854_s_at | MAP3K7 | TAK1, TGF1a | mitogen-activated | 6885 | GCATGCTCATTGTGTGAAGGCTAGC (SEQ ID NO: 1955) |
| | | | protein kinase kinase | | GGAGGTATCAAACTAGCTGCTATGT (SEQ ID NO: 1956) |
| | | | kinase 7 | | CTGCTATGTGCAAACAGCGTCCATT (SEQ ID NO: 1957) |
| | | | | | ACAGCGTCCATTTTTTCATATTAGA (SEQ ID NO: 1958) |
| | | | | | GGTGGAACCTCAAGAATGACTTTAT (SEQ ID NO: 1959) |
| | | | | | ATGACTTTATTCTTGTATCTCATCT (SEQ ID NO: 1960) |
| | | | | | GGGATAGTTCCGTGTAAGGGCTTTG (SEQ ID NO: 1961) |
| | | | | | GGCTTTGATGCCAGCATCCTTGGAT (SEQ ID NO: 1962) |
| | | | | | AGCATCCTTGGATCAGTACTGAACT (SEQ ID NO: 1963) |
| | | | | | TGAACTCAGTTCCATCCGTAAAATA (SEQ ID NO: 1964) |
| | | | | | GGTAAGTGGCAGCTGCTCTATTTAA (SEQ ID NO: 1965) |
| 243_g_at | MAP4 | DKFZp779A1753, | microtubule- | 4134 | TTCTTTTCTAACCAAGAGGCTGGTT (SEQ ID NO: 1966) |
| | | MGC8617 | associated protein 4 | | TTGGCATCTCTGTCCCATTCCCTGG (SEQ ID NO: 1967) |
| | | | | | AGATGGAATTCCTTCAGGCCGGCAC (SEQ ID NO: 1968) |
| | | | | | TCCAAGGGAGCCTCATGCAGATAGT (SEQ ID NO: 1969) |
| | | | | | AAGGGAGCCTCATGCAGATAGTAGG (SEQ ID NO: 1970) |
| | | | | | GAGCCTCATGCAGATAGTAGGAAAT (SEQ ID NO: 1971) |
| | | | | | CCTCATGCAGATAGTAGGAAATCAG (SEQ ID NO: 1972) |
| | | | | | GAGACTAAGGCTATCTGCTTCCCTT (SEQ ID NO: 1973) |
| | | | | | ACTAAGGCTATCTGCTTCCCTTATG (SEQ ID NO: 1974) |
| | | | | | AAGGCTATCTGCTTCCCTTATGCCC (SEQ ID NO: 1975) |
| | | | | | TCTGCTTCCCTTATGCCCTGACTGG (SEQ ID NO: 1976) |
| | | | | | AGGGCATCCCAGCTAGGCCTTGGGA (SEQ ID NO: 1977) |
| | | | | | GAGAAATCCCGGGAACTGTATTGAC (SEQ ID NO: 1978) |
| | | | | | AATCCCGGGAACTGTATTGACACAA (SEQ ID NO: 1979) |
| | | | | | ATTGACACAAAGATTCTTATTGCAC (SEQ ID NO: 1980) |
| | | | | | ACACAAAGATTCTTATTGCACTTGT (SEQ ID NO: 1981) |
| 218181_s_at | MAP4K4 | FLH21957, | mitogen-activated | 9448 | GTAACTTCTCCACAGAAGTGCCAAT (SEQ ID NO: 1982) |
| | | FLJ10410, | protein kinase kinase | | AAACAGTATTGCAATGACACCATTA (SEQ ID NO: 1983) |
| | | FLJ20373, | kinase kinase 4 | | TGTTAGAGGAGTGCTCTTCCAAACA (SEQ ID NO: 1984) |
| | | FLJ90111, HGK, | | | AGGTTTAGTCAGAGCTTTCACAAGT (SEQ ID NO: 1985) |
| | | KIAA0687, NIK | | | GAGTAACCCTTTCTGTATTGAGTGC (SEQ ID NO: 1986) |
| | | | | | GTTTTGTTACACTAATGCGCACCAC (SEQ ID NO: 1987) |
| | | | | | CACTAATGCGCACCACATATTTATG (SEQ ID NO: 1988) |
| | | | | | GGTTTTCGTCTTAGTTGTAGCACAC (SEQ ID NO: 1989) |
| | | | | | GTAGCACACTTACCCTAATTTTGCC (SEQ ID NO: 1990) |
| | | | | | GACTGGTGGCAAGTACACAGCCCTT (SEQ ID NO: 1991) |
| | | | | | CCCTTTTTTTTGCAGTGCTAACTTG (SEQ ID NO: 1992) |
| 203552_at | MAP4K5 | GCKR, KHS, | mitogen-activated | 11183 | ACACACACATGCAATTTTGCTTAAC (SEQ ID NO: 1993) |
| | | KHS1, | protein kinase kinase | | GAAGGGGTGTGTATGTCTAGCTGTG (SEQ ID NO: 1994) |
| | | MAPKKKK5 | kinase kinase 5 | | ATGTCTAGCTGTGTCTACCATGTGT (SEQ ID NO: 1995) |
| | | | | | GTCTACCATGTGTATGTATTCTTGA (SEQ ID NO: 1996) |
| | | | | | AATCTTCATATATATTATCATCCCT (SEQ ID NO: 1997) |
| | | | | | ATATTATCATCCCTAATGTAGCAGG (SEQ ID NO: 1998) |
| | | | | | GGGAAGTATTTAATTGCCCATGATA (SEQ ID NO: 1999) |
| | | | | | ATTTTACTTATACTATGCCAGAGAG (SEQ ID NO: 2000) |
| | | | | | TTACACATGTAATCTTGGGTTTTTC (SEQ ID NO: 2001) |
| | | | | | GTTTTTCACATATGTAGGTATTCAT (SEQ ID NO: 2002) |
| | | | | | GAATTCCTGATGGGATAGTATCAAT (SEQ ID NO: 2003) |
| 212871_at | MAPKAPK5 | MAPKAPK5, | mitogen-activated | 8550 | TGCCTTCTGCTCAGCTGATGATGGA (SEQ ID NO: 2004) |
| | | PRAK | protein kinase- | | GTTGCAGGAATCCAGCAGGCTCACG (SEQ ID NO: 2005) |
| | | | activated protein | | GGCTCACGCGGAACAGTTGGCCAAC (SEQ ID NO: 2006) |
| | | | kinase 5 | | GGCCAACATGAGAATCCAGGATCTG (SEQ ID NO: 2007) |
| | | | | | ACAACCCCATTCTGCGGAAGAGGAA (SEQ ID NO: 2008) |
| | | | | | AGAGGAAGTTACTTGGCACCAAGCC (SEQ ID NO: 2009) |
| | | | | | GACAGTGTCTATATCCACGACCATG (SEQ ID NO: 2010) |
| | | | | | GGAGCCGAGGATTCCAATGTTGCCT (SEQ ID NO: 2011) |
| | | | | | ATGTGATTGCTCAGTGTATTCTCCC (SEQ ID NO: 2012) |
| | | | | | CCCACGAATCCCAATAATGACAGCT (SEQ ID NO: 2013) |
| | | | | | ATGACAGCTTCAGACTTTGTTTTTT (SEQ ID NO: 2014) |
| 205018_s_at | MBNL2 | RP11-128N14.1, | muscleblind-like 2 | 10150 | CCTGTTTCCTCCTCAATAGAATTGC (SEQ ID NO: 2015) |
| | | DKFZp781H1296, | (Drosophila) | | GTTCTTCTGGACAGACCACAACTCT (SEQ ID NO: 2016) |
| | | MBLL, MBLL39, | | | GAAGCTAGTGCTGCTATCTCATATA (SEQ ID NO: 2017) |
| | | MGC120625, | | | GATAGTTAACTACCTGAGACCAGCT (SEQ ID NO: 2018) |
| | | MGC120626, | | | AGGAATATTGTCTTATCTCCATAAC (SEQ ID NO: 2019) |
| | | MGC120628, | | | CAAGGGCAGGTTCGATTCTAGCTCT (SEQ ID NO: 2020) |
| | | PRO2032 | | | TTCTAGCTCTAATTACTGTCATGTC (SEQ ID NO: 2021) |
| | | | | | TACTGTCATGTCATTTACCCACTGG (SEQ ID NO: 2022) |
| | | | | | GGTATGTTTCACTTCTTGACAATAT (SEQ ID NO: 2023) |
| | | | | | AACCGATACCTGTCCTGCAGGTCTA (SEQ ID NO: 2024) |
| | | | | | CAAGCACAAGTACTGGGGACACATC (SEQ ID NO: 2025) |
| 201755_at | MCM5 | CDC46, | minichromosome | 4174 | CATCGCGGAAGCCCTCAGCAAGATG (SEQ ID NO: 2026) |
| | | MGC5315, P1- | maintenance | | GCTCTTCCAAGTGTCCACGTTGGAT (SEQ ID NO: 2027) |
| | | CDC46 | complex component 5 | | TTGTCCGGTACCCTGTCAGGGGTGG (SEQ ID NO: 2028) |
| | | | | | TGTCAGGGGTGGAGGGCTTCACCAG (SEQ ID NO: 2029) |
| | | | | | GAGGACCAGGAGATGCTGAGCCGCA (SEQ ID NO: 2030) |
| | | | | | ATGCTGAGCCGCATCGAGAAGCAGC (SEQ ID NO: 2031) |
| | | | | | CATCGAGAAGCAGCTCAAGCGCCGC (SEQ ID NO: 2032) |
| | | | | | TCCCAGGTGTCTGAGCACAGCATCA (SEQ ID NO: 2033) |
| | | | | | GCACAGCATCATCAAGGACTTCACC (SEQ ID NO: 2034) |
| | | | | | GGACTTCACCAAGCAGAAATACCCG (SEQ ID NO: 2035) |
| | | | | | CGCCCGCTCTAGCGGGTTCTGGGAA (SEQ ID NO: 2036) |
| 216237_s_at | MCM5 | CDC46, | minichromosome | 4174 | CGGCGAGATCCAGCATCGCATGCAG (SEQ ID NO: 2037) |
| | | MGC5315, P1- | maintenance | | GCGAGATCCAGCATCGCATGCAGCG (SEQ ID NO: 2038) |
| | | CDC46 | complex component 5 | | CCAGCATCGCATGCAGCGCAAGGTT (SEQ ID NO: 2039) |
| | | | | | GCATCGCATGCAGCGCAAGGTTCTC (SEQ ID NO: 2040) |
| | | | | | TCGCATGCAGCGCAAGGTTCTCTAC (SEQ ID NO: 2041) |
| | | | | | CTGCCTGCCATTGACAATGTTGCTG (SEQ ID NO: 2042) |
| | | | | | TTGACAATGTTGCTGGGACCTCTGC (SEQ ID NO: 2043) |
| | | | | | GAGGAAGGAGCTGTAGTGTCCTGCT (SEQ ID NO: 2044) |
| | | | | | GAAGGAGCTGTAGTGTCCTGCTGCC (SEQ ID NO: 2045) |
| | | | | | GTTCTGGGAAGTGTGCTTTTGGCAT (SEQ ID NO: 2046) |
| | | | | | CTGGGAAGTGTGCTTTTGGCATCCG (SEQ ID NO: 2047) |
| 202556_s_at | MCRS1 | ICP22BP, | microspherule protein 1 | 10445 | GGGTCCGGCCTGGAAGATATCCCGG (SEQ ID NO: 2048) |
| | | INO80Q, | | | GAACAACGGTGATTTCTTCATTGCC (SEQ ID NO: 2049) |
| | | MCRS2, MSP58, | | | TGCCAATGAGGGTCGACGGCCCATC (SEQ ID NO: 2050) |
| | | P78 | | | GACGGCCCATCTACATCGATGGACG (SEQ ID NO: 2051) |
| | | | | | CGCCTCAGCAACAACTCTGTGGTGG (SEQ ID NO: 2052) |
| | | | | | TCGTCTTCCTTATCAACCAGGACCT (SEQ ID NO: 2053) |
| | | | | | AGGAGTGGTGGCAGGACTCGTGGGC (SEQ ID NO: 2054) |
| | | | | | TGAGCTGGGAACTCAGGCTCCTGGA (SEQ ID NO: 2055) |
| | | | | | GCTGCGGGCCATTGATTTGAGCCTT (SEQ ID NO: 2056) |
| | | | | | GAGGGAGGATAGGGCTGGCCTTTGT (SEQ ID NO: 2057) |
| | | | | | GTCTCCAGCTGATTAGCCTCAGACT (SEQ ID NO: 2058) |
| 219051_x_at | METRN | MGC2601; | meteorin, glial cell | 79006 | CGCTTTGAGCTGCGCGAGGACGGGC (SEQ ID NO: 2059) |
| | | C16orf23; | differentiation | | GCACCAGCGACTTCGTAATTCACGG (SEQ ID NO: 2060) |
| | | c380A1.2 | regulator | | CAGCGACTTCGTAATTCACGGGATC (SEQ ID NO: 2061) |
| | | | | | TCACGGGATCATCCATGGGGTCACC (SEQ ID NO: 2062) |
| | | | | | TCACCCATGACGTGGAGCTGCAGGA (SEQ ID NO: 2063) |
| | | | | | GCTGCAGGAGTCTGTCATCACTGTG (SEQ ID NO: 2064) |
| | | | | | TCATGGGCTGGAGCCGCTTTGGGGA (SEQ ID NO: 2065) |
| | | | | | GCTGCACTGAGGGGCTGGGTGCTGG (SEQ ID NO: 2066) |
| | | | | | TGCTGGGGAGGGGCTGGTAGGAGGG (SEQ ID NO: 2067) |
| | | | | | TGGTAGGAGGGAGGGTGGGCCCACT (SEQ ID NO: 2068) |
| | | | | | GGCCCACTGCTTTGGAGGTGATGGG (SEQ ID NO: 2069) |
| 213653_at | METTL3 | M6A, MGC4336, | methyltransferase | 56339 | CTGTTATCTGTGGTGAGCAGGTTGG (SEQ ID NO: 2070) |
| | | MT-A70, Spo8 | like 3 | | ATCTGTGGTGAGCAGGTTGGTGTCA (SEQ ID NO: 2071) |
| | | | | | GTGGTGAGCAGGTTGGTGTCAAAGG (SEQ ID NO: 2072) |
| | | | | | GATCGTAGCTGAGGTATGTGCTTCC (SEQ ID NO: 2073) |
| | | | | | TAGCTGAGGTATGTGCTTCCCAGGC (SEQ ID NO: 2074) |
| | | | | | CCTCCAAAGCTTCCACATTTTTGTT (SEQ ID NO: 2075) |
| | | | | | GGGTGTATTCTCATCCCAGATTTTT (SEQ ID NO: 2076) |
| | | | | | GATTTTTCTCATTTAGATCATAAAC (SEQ ID NO: 2077) |
| | | | | | GAAAGGGCTAGAATTGCAATCTTGT (SEQ ID NO: 2078) |
| | | | | | GGCTAGAATTGCAATCTTGTGTAAC (SEQ ID NO: 2079) |
| | | | | | GAATTGCAATCTTGTGTAACTTAAA (SEQ ID NO: 2080) |
| 213457_at | MFHAS1 | FLJ23354, | malignant fibrous | 9258 | TCACCCCAGCGTGTTCTGTGAACTT (SEQ ID NO: 2081) |
| | | LRRC65, MASL1 | histiocytoma | | TGACAACGCGTGCTTGCAGGGTGCT (SEQ ID NO: 2082) |
| | | | amplified sequence 1 | | AGGGTGCTTTTTGGATGACTGGGGA (SEQ ID NO: 2083) |
| | | | | | GAACCTCAGAACTGTGATCCTCCAA (SEQ ID NO: 2084) |
| | | | | | TCCTCCAAGGAGCGCGCTACTTGAA (SEQ ID NO: 2085) |
| | | | | | GTCGAATGGCTTCTCAGGGATTTTG (SEQ ID NO: 2086) |
| | | | | | GGGATTTTGTTTTCCGTGCACATAT (SEQ ID NO: 2087) |
| | | | | | AGAGCACTCAGATTTCAGTTCTGTT (SEQ ID NO: 2088) |
| | | | | | GAGGGATCGACTGACGTTCATTGCT (SEQ ID NO: 2089) |
| | | | | | GACGTTCATTGCTGTTCCATAACTA (SEQ ID NO: 2090) |
| | | | | | GAAGCTTCTAGCGTTAGTTCACACT (SEQ ID NO: 2091) |
| 207013_s_at | MMP16 | MMP-X2, MT- | matrix | 4325 | CTATCCCAAGCCAATCACAGTCTGG (SEQ ID NO: 2092) |
| | | MMP2, MT- | metallopeptidase 16 | | TCCTCAGGGAGCATTTGTACACAAA (SEQ ID NO: 2093) |
| | | MMP3, MT3- | (membrane-inserted) | | GACACAGCCCACCAGATGATGTAGA (SEQ ID NO: 2094) |
| | | MMP | | | AACTGGACAACACAGCCAGCACTGT (SEQ ID NO: 2095) |
| | | | | | AGCCATAGCTATTGTCATTCCCTGC (SEQ ID NO: 2096) |
| | | | | | ATGCCTCCTTGTATTGGTTTACACT (SEQ ID NO: 2097) |
| | | | | | TACTGTACTGTAAACGCTCTATGCA (SEQ ID NO: 2098) |
| | | | | | GAGTGGGTGTGATGTAGGGTTTTTT (SEQ ID NO: 2099) |
| | | | | | GCTGTTATGCTGTTTCCTAGCTAGG (SEQ ID NO: 2100) |
| | | | | | TAGCTAGGAGCAGGCTTGTGGCAGC (SEQ ID NO: 2101) |
| | | | | | TTGTGGCAGCCTGATTCGGGGCTGA (SEQ ID NO: 2102) |
| 205828_at | MMP3 | MGC126102, | matrix | 4314 | GAAAATCGATGCAGCCATTTCTGAT (SEQ ID NO: 2103) |
| | | MGC126103, | metallopeptidase 3 | | TTTATTTCTTTACTGGATCTTCACA (SEQ ID NO: 2104) |
| | | MGC126104, | (stromelysin 1, | | GATCTTCACAGTTGGAGTTTGACCC (SEQ ID NO: 2105) |
| | | MMP-3, SL-1, | progelatinase) | | TAATTCTTCACCTAAGTCTCTGTGA (SEQ ID NO: 2106) |
| | | STMY, STMY1, | | | ATTGAAATGTTCGTTTTCTCCTGCC (SEQ ID NO: 2107) |
| | | STR1 | | | GTGACTCGAGTCACACTCAAGGGAA (SEQ ID NO: 2108) |
| | | | | | TGAGCGTGAATCTGTATCTTGCCGG (SEQ ID NO: 2109) |
| | | | | | GTATCTTGCCGGTCATTTTTATGTT (SEQ ID NO: 2110) |
| | | | | | CAAATGGGCTGCTGCTTAGCTTGCA (SEQ ID NO: 2111) |
| | | | | | TTAGCTTGCACCTTGTCACATAGAG (SEQ ID NO: 2112) |
| | | | | | GGGGAAGCACTCGTGTGCAACAGAC (SEQ ID NO: 2113) |
| 218853_s_at | MOSPD1 | RP3-473B4.2, | motile sperm domain | 56180 | GTCCCCTAAGCACAACTATTTACAT (SEQ ID NO: 2114) |
| | | DJ473B4 | containing 1 | | TACATTTCTTTATAGCCTCTTCTGA (SEQ ID NO: 2115) |
| | | | | | TTATAGCCTCTTCTGATCTCTAACA (SEQ ID NO: 2116) |
| | | | | | GTAACTGATCTTTTGTCTAAGGATT (SEQ ID NO: 2117) |
| | | | | | GAACTTATGCACTGCTACTGTAGTA (SEQ ID NO: 2118) |
| | | | | | AACAGTCTCTGCTTCTAAAAACTTC (SEQ ID NO: 2119) |
| | | | | | AAAACTTCTATGTCTAGTCTTCCAT (SEQ ID NO: 2120) |
| | | | | | TAGGAAATCCTCACTGTTTAACCAT (SEQ ID NO: 2121) |
| | | | | | GGAGCCTAAGTCATTAAACGGATCA (SEQ ID NO: 2122) |
| | | | | | AACGGATCATGTCTGTACATTGTGT (SEQ ID NO: 2123) |
| | | | | | GGAGGCTATTCTTGTTTCTCCATCT (SEQ ID NO: 2124) |
| 64883_at | MOSPD2 | MGC26706 | motile sperm domain | 158747 | CAGTTGTCTGCCTGTAAAATGTTGA (SEQ ID NO: 2125) |
| | | | containing 2 | | CTGCCTGTAAAATGTTGAGTTTCGA (SEQ ID NO: 2126) |
| | | | | | GATATAACAGTTCCTTCAAAATTGG (SEQ ID NO: 2127) |
| | | | | | TTCCTTCAAAATTGGCCTAGGAAAT (SEQ ID NO: 2128) |
| | | | | | CACCTCCAGCTGTGAAATGGACTGC (SEQ ID NO: 2129) |
| | | | | | TCCAGCTGTGAAATGGACTGCAGTC (SEQ ID NO: 2130) |
| | | | | | TGAAATGGACTGCAGTCCACCCTAA (SEQ ID NO: 2131) |
| | | | | | CCTGTGTGTGTGCACAGTGGCTTCC (SEQ ID NO: 2132) |
| | | | | | TATGTTTTCTGTTCCTTGGCAAATA (SEQ ID NO: 2133) |
| | | | | | GTTTTCTGTTCCTTGGCAAATAAAT (SEQ ID NO: 2134) |
| | | | | | CTGTTCCTTGGCAAATAAATGAAGA (SEQ ID NO: 2135) |
| | | | | | AATGTATTGTCCTAACGGTGTCCCT (SEQ ID NO: 2136) |
| | | | | | TGTCCTAACGGTGTCCCTTTAATGT (SEQ ID NO: 2137) |
| | | | | | TAACGGTGTCCCTTTAATGTTTCAT (SEQ ID NO: 2138) |
| | | | | | GTGTCCCTTTAATGTTTCATATGAA (SEQ ID NO: 2139) |
| | | | | | AATATCCTTGCTCAATGTCTGGTCA (SEQ ID NO: 2140) |
| 221771_s_at | MPHOSPH8 | RP11-523H24.1, | M-phase | 54737 | CTATACTCTCTTTGACAGCAGTTTG (SEQ ID NO: 2141) |
| | | FLJ35237, | phosphoprotein 8 | | GACAGCAGTTTGGAATTCTTCTAGC (SEQ ID NO: 2142) |
| | | HSMPP8, TWA3, | | | GGAATTCTTCTAGCACATCTATGTA (SEQ ID NO: 2143) |
| | | mpp8 | | | AGTTTTGTCTGTAAACCTCTTGCAG (SEQ ID NO: 2144) |
| | | | | | GTGTCTGTGCCAGTATGCCGGAATC (SEQ ID NO: 2145) |
| | | | | | TGCCAGTATGCCGGAATCTCAGTGC (SEQ ID NO: 2146) |
| | | | | | GCCGGAATCTCAGTGCAGTGTCCAG (SEQ ID NO: 2147) |
| | | | | | GGAATCTCAGTGCAGTGTCCAGACT (SEQ ID NO: 2148) |
| | | | | | CAGTGTCCAGACTGCGTATTTCAGT (SEQ ID NO: 2149) |
| | | | | | TGCGTATTTCAGTTTTTCCACAATG (SEQ ID NO: 2150) |
| | | | | | GTGGATAGTACATATGAGGATTATT (SEQ ID NO: 2151) |
| 217980_s_at | MRPL16 | FLJ20484, | mitochondrial | 54948 | TTGGGCATCGCATGGGGGGAGGCAA (SEQ ID NO: 2152) |
| | | L16mt, MRP-L16, | ribosomal protein L16 | | GTGCTATTGACCACTACGTGACACC (SEQ ID NO: 2153) |
| | | PNAS-111 | | | ACGTGACACCTGTGAAGGCTGGCCG (SEQ ID NO: 2154) |
| | | | | | GCAAGGTTTCCTTGACCAGGTTGCC (SEQ ID NO: 2155) |
| | | | | | TTGCCCTTCGCAGCAAAGGCTGTGA (SEQ ID NO: 2156) |
| | | | | | CTGTGAGCCGCGGGACTCTAGAGAA (SEQ ID NO: 2157) |
| | | | | | AGAACCCCTGGACATTTGAGCGAAT (SEQ ID NO: 2158) |
| | | | | | AGCGAATAGCCACTGCCAACATGCT (SEQ ID NO: 2159) |
| | | | | | GTACTGAGCCCATATGACTTGACCC (SEQ ID NO: 2160) |
| | | | | | TCAGCCTACCCACTGAAGTCTTTGG (SEQ ID NO: 2161) |
| | | | | | GTCTTTGGGTAGCTCTTAAGCCATA (SEQ ID NO: 2162) |
| 203152_at | MRPL40 | FLJ41774, | mitochondrial | 64976 | TATTACCCCTCTAAAGTTCTTGGAT (SEQ ID NO: 2163) |
| | | MGC9400, MRP- | ribosomal protein L40 | | AGCAAGAGAGCGGCCTCAGGTGGAG (SEQ ID NO: 2164) |
| | | L22, NLVCF, | | | GGAGCTCACCTTTGAGGAGACTGAG (SEQ ID NO: 2165) |
| | | URIM | | | GACACCATCAGGGCTATGCTAGAAG (SEQ ID NO: 2166) |
| | | | | | GAAGCTCTGGAGGAACTGCAACTGG (SEQ ID NO: 2167) |
| | | | | | AACTGCAACTGGAATCCCCGAAGCT (SEQ ID NO: 2168) |
| | | | | | TCCATGCTGAGGCCATCAAGCGGGA (SEQ ID NO: 2169) |
| | | | | | ATCAAGCGGGATCCTAACCTGTTCC (SEQ ID NO: 2170) |
| | | | | | GAAGGAAGGGCCACATTACACACCA (SEQ ID NO: 2171) |
| | | | | | GGCTGCTATCCTTAACATGCTGCCC (SEQ ID NO: 2172) |
| | | | | | TTAACATGCTGCCCCTGAGAGTAGG (SEQ ID NO: 2173) |
| 219244_s_at | MRPL46 | LIECG2; | mitochondrial | 26589 | GGAACCTTGTCCTGTTAGTCAGAGA (SEQ ID NO: 2174) |
| | | P2ECSL; | ribosomal protein L46 | | CAGGATGTTTGGATACTGCCCCAGG (SEQ ID NO: 2175) |
| | | C15orf4; | | | GCCTGGGGAGACCCTTCGAGGAACA (SEQ ID NO: 2176) |
| | | MGC22762 | | | GAAATGCACCCTGTGGGCACTACAC (SEQ ID NO: 2177) |
| | | | | | GTAACCTCGGAGCCAAGGIGTTCTT (SEQ ID NO: 2178) |
| | | | | | GGTGTTCTTCTTCAAAGCACTGCTA (SEQ ID NO: 2179) |
| | | | | | ACTGGAGACTTTTCCCAGGCTGGGA (SEQ ID NO: 2180) |
| | | | | | AAGGGCCATCATGTGTGGGTCACTA (SEQ ID NO: 2181) |
| | | | | | TTTCAGACCTCTGATGGGCCGAGCT (SEQ ID NO: 2182) |
| | | | | | GTGGACGGTGCTCAGACAAGTCTGG (SEQ ID NO: 2183) |
| | | | | | GTGATTGCCTCACATTTGCAGGTAA (SEQ ID NO: 2184) |
| 211595_s_at | MRPS11 | FLJ22512, | mitochondrial | 64963 | ACACAGATCCAGGTAGTCTCTGCTA (SEQ ID NO: 2185) |
| | | FLJ23406, HCC-2 | ribosomal protein | | GTCTCTGCTAGTAATGAGCCCCTTG (SEQ ID NO: 2186) |
| | | | S11 | | AGCACAGACAGCAGGCATAGCCGCA (SEQ ID NO: 2187) |
| | | | | | AGCCGCAGCGGCGAGAGCTAAACAA (SEQ ID NO: 2188) |
| | | | | | CAAAAGGGCGTGATCCACATCCGAG (SEQ ID NO: 2189) |
| | | | | | TTGTCTGCCATGCACGGACTGATCA (SEQ ID NO: 2190) |
| | | | | | GGACTGATCATGGGCGGCCTGGAAG (SEQ ID NO: 2191) |
| | | | | | CTCAGCTCCAGTGGGACCTTGTAAA (SEQ ID NO: 2192) |
| | | | | | GGACCTTGTAAAATGCTCCCTGTCA (SEQ ID NO: 2193) |
| | | | | | TCCCTGTCAGAGCTCTCCAGAATAT (SEQ ID NO: 2194) |
| | | | | | GGTGTGCCCCAGAAGTAAGCTTTGC (SEQ ID NO: 2195) |
| 212096_s_at | MTUS1 | ATIP; MP44; | mitochondrial tumor | 57509 | TGGGTCATTTACATGTACACTACAT (SEQ ID NO: 2196) |
| | | MTSG1; | suppressor 1 | | GATACTCATGTTGCATGTTCTTTTA (SEQ ID NO: 2197) |
| | | FLJ14295; | | | TAGTGATTTTGTGTCTTAAGTCTTT (SEQ ID NO: 2198) |
| | | KIAA1288; | | | TAAGTCTTTAACTTCCAATACTTCA (SEQ ID NO: 2199) |
| | | DKFZp586D1519; | | | GTATGTAACCTTCCATGTTTGCTTC (SEQ ID NO: 2200) |
| | | DKFZp686F20243 | | | GGAAATGTAGGTTCACTGCCACTTC (SEQ ID NO: 2201) |
| | | | | | GGTTCACTGCCACTTCATGAGATAT (SEQ ID NO: 2202) |
| | | | | | CATGAGATATCTCTGCTCACGCTTC (SEQ ID NO: 2203) |
| | | | | | CAAAGTTGGGTTTGCCATTCATCCC (SEQ ID NO: 2204) |
| | | | | | ATGGTAAATCTTGTGTTGTTCCCTG (SEQ ID NO: 2205) |
| | | | | | TGTCCTCCGTATTACGTGACCGGCA (SEQ ID NO: 2206) |
| 204056_s_at | MVK | FLJ96772, LRBP, | mevalonate kinase | 4598 | GCTCCTGACACTGCTGGAGAGGCCC (SEQ ID NO: 2207) |
| | | MK, MVLK | | | GCAGTCCCAGCGGTGGGACCTAGGG (SEQ ID NO: 2208) |
| | | | | | ACCTAGGGAGGCATGGTCTGCCCTC (SEQ ID NO: 2209) |
| | | | | | CCAGCCGAGCAGGAGGCCTAGGAGG (SEQ ID NO: 2210) |
| | | | | | GCCTAGGAGGGTCCTCTGAGACTCC (SEQ ID NO: 2211) |
| | | | | | CTGAGACTCCAGACCTGAGGCGAGA (SEQ ID NO: 2212) |
| | | | | | TGAGGCGAGAAGGGCTGCTTCCCTG (SEQ ID NO: 2213) |
| | | | | | CCTGGGTCCAATGCTCAGGTGCTGG (SEQ ID NO: 2214) |
| | | | | | GGGCCTGGTTCCCGGAGAAGTGTGC (SEQ ID NO: 2215) |
| | | | | | CCTTCTACCTAGCGGGATGGGGCTC (SEQ ID NO: 2216) |
| | | | | | CTTTCTTCCGATCTGTACCTGAGAG (SEQ ID NO: 2217) |
| 203359_s_at | MYCBP | AMY-1 | c-myc binding protein | 26292 | TGTGCTGTTTTTCAATTTGCCTAAT (SEQ ID NO: 2218) |
| | | | | | GCCTGCCTTATTTGCTTACTGAGCA (SEQ ID NO: 2219) |
| | | | | | CAACTCTGGGTTCACACACAAGCTG (SEQ ID NO: 2220) |
| | | | | | AATTATATAGTGCTTTTCTCCCTCA (SEQ ID NO: 2221) |
| | | | | | TCTCCCTCAAAGTATTTTTCTAGCC (SEQ ID NO: 2222) |
| | | | | | ATTCATTTTATCTTCATTATCCCTG (SEQ ID NO: 2223) |
| | | | | | GAGGGGTGCTGAATTTTTAGGCCAA (SEQ ID NO: 2224) |
| | | | | | ATCACTCACTAACTGTAGAGCCTTG (SEQ ID NO: 2225) |
| | | | | | GAGCCTTGGGCATTATCAGTGAACT (SEQ ID NO: 2226) |
| | | | | | TACTCTGAGATTTACTGTCTTCATC (SEQ ID NO: 2227) |
| | | | | | TAATGAGTAGAATGTCCGTGATGCC (SEQ ID NO: 2228) |
| 214156_at | MYRIP | DKFZp586F1018, | myosin VIIA and Rab | 25924 | GGCTTTTTGTTCTGTTGGTGTGATT (SEQ ID NO: 2229) |
| | | FLJ44025, | interacting protein | | TGACTTCCGAATAGAATGTTTTCTT (SEQ ID NO: 2230) |
| | | MGC130034, | | | GCAACTATTGTTGTGTTACAGTGTT (SEQ ID NO: 2231) |
| | | MGC130035, | | | GTGTACTGTGTAAGCCTTGCAAACA (SEQ ID NO: 2232) |
| | | SLAC2-C, | | | AGCCTGCTGTGTGGCATCTGAACTT (SEQ ID NO: 2233) |
| | | SLAC2C | | | AGGTTTCCTTGTGCCAAATAAGTGC (SEQ ID NO: 2234) |
| | | | | | GCATATGTTATAGTTCCAGAAGAAT (SEQ ID NO: 2235) |
| | | | | | GGATAATATCTTTGATGACTTCTGA (SEQ ID NO: 2236) |
| | | | | | GATGACTTCTGAAAGTTATGCTTCC (SEQ ID NO: 2237) |
| | | | | | TTATGCTTCCCTTCATGTTATATGC (SEQ ID NO: 2238) |
| | | | | | CACATTGCCAAGAATTACTGTCAAG (SEQ ID NO: 2239) |
| 202259_s_at | N4BP2L2 | RP11-298P3.3, | NEDD4 binding | 10443 | TTTTTCCTTCACTACCTTAAATATG (SEQ ID NO: 2240) |
| | | 92M18.3, CG005, | protein 2-like 2 | | GGGTACCTTAGTTTTCTCTATTCAT (SEQ ID NO: 2241) |
| | | FLJ36195, | | | ATTTCAGCTGATTGATCTGTGTGAC (SEQ ID NO: 2242) |
| | | FLJ41089, | | | ATAATCAGCACATCACTTAGTTCAA (SEQ ID NO: 2243) |
| | | FLJ43077, | | | ACAAAATTCCCCAGCAAATGTTAGA (SEQ ID NO: 2244) |
| | | PFAAP5 | | | GTAGGTATATCAGTCACCTGGGGAG (SEQ ID NO: 2245) |
| | | | | | TGGGGAGTTTTCTTCATAATATGCA (SEQ ID NO: 2246) |
| | | | | | CATACATAGTTATCATCCTCCTTCT (SEQ ID NO: 2247) |
| | | | | | CCACATGCTTGCCAGTTCTTGAAGG (SEQ ID NO: 2248) |
| | | | | | TGGACAGTTGCTTAACATGGTCATA (SEQ ID NO: 2249) |
| | | | | | ATATTTCTCTCACTGGATAGTTTTT (SEQ ID NO: 2250) |
| 209272_at | NAB1 | | NGFI-A binding | 4664 | ACGTTTCCTGTATTCTAATCTATTT (SEQ ID NO: 2251) |
| | | | protein 1 (EGR1 | | ATCTTCCAACTTCCAATATTTATCC (SEQ ID NO: 2252) |
| | | | binding protein 1) | | TTTATCCATTCGTTGTGGACCCACA (SEQ ID NO: 2253) |
| | | | | | GTGGACCCACAGATTGCATCTTTAA (SEQ ID NO: 2254) |
| | | | | | TTATGTTTCTAGTCTTTCAAGCTTA (SEQ ID NO: 2255) |
| | | | | | GGTTACCTCAGTATTACAGCCAATA (SEQ ID NO: 2256) |
| | | | | | TATAGTCCAAGGGACCATTTCTCCC (SEQ ID NO: 2257) |
| | | | | | CCCGAGTCTCTTACACTTTATTGTG (SEQ ID NO: 2258) |
| | | | | | GATGTCCACGTTTTTGTGACTCTTC (SEQ ID NO: 2259) |
| | | | | | GAGGTGGGACGAATGCACTTGCTTC (SEQ ID NO: 2260) |
| | | | | | TGCACTTGCTTCCTGTGGCAATAAA (SEQ ID NO: 2261) |
| 218189_s_at | NANS | RP11-404F11.3, | N-acetylneuraminic | 54187 | CTTTCCTGACATTCCCATAGGGTAT (SEQ ID NO: 2262) |
| | | SAS | acid synthase (sialic | | AGCGATATCTGTGGCCGCAGTGGCT (SEQ ID NO: 2263) |
| | | | acid synthase) | | GGCTCTGGGGGCCAAGGTGTTGGAA (SEQ ID NO: 2264) |
| | | | | | GGAACGTCACATAACTTTGGACAAG (SEQ ID NO: 2265) |
| | | | | | GAGCCTGGAGAACTGGCCGAGCTGG (SEQ ID NO: 2266) |
| | | | | | AATTCCGGAAGGCACCATTCTAACA (SEQ ID NO: 2267) |
| | | | | | TTCTAACAATGGACATGCTCACCGT (SEQ ID NO: 2268) |
| | | | | | AAGGCTATCCTCCTGAAGACATCTT (SEQ ID NO: 2269) |
| | | | | | GAAGACATCTTTAATCTAGTGGGCA (SEQ ID NO: 2270) |
| | | | | | GTGGGCAAGAAGGTCCTGGTCACTG (SEQ ID NO: 2271) |
| | | | | | GAAGGTCCTGGTCACTGTTGAAGAG (SEQ ID NO: 2272) |
| 200027_at | NARS | ASNRS, NARS1 | asparaginyl-tRNA | 4677 | ATCTTTTCTAACTCTGCTTAGCTGC (SEQ ID NO: 2273) |
| | | | synthetase | | AGCTGCTAATAATCCTGAGGCATAG (SEQ ID NO: 2274) |
| | | | | | TAGTGATTCACAGTATCCCTCTTAG (SEQ ID NO: 2275) |
| | | | | | AGTATCCCTCTTAGCATTAATTTAA (SEQ ID NO: 2276) |
| | | | | | GAGGCAGATTGATTTTCCCTCTTTC (SEQ ID NO: 2277) |
| | | | | | CCCACCATGTCCTTAGATCTAATCT (SEQ ID NO: 2278) |
| | | | | | TAGATCTAATCTGTGCTACCTTATT (SEQ ID NO: 2279) |
| | | | | | GCTACCTTATTAACTCACAGCAGGC (SEQ ID NO: 2280) |
| | | | | | ACAGCAGGCTTACTGAATGGCTTCA (SEQ ID NO: 2281) |
| | | | | | CAGATTTAGTTGATTTCTCCACCAA (SEQ ID NO: 2282) |
| | | | | | GCATGTCATGTATTCTCAATAGGCT (SEQ ID NO: 2283) |
| 214440_at | NAT1 | AAC1, NATI | N-acetyltransferase 1 | 9 | GATGGGGTTCACTGTTTGGTGGGCT (SEQ ID NO: 2284) |
| | | | (arylamine N- | | TTCACCCTCACCCATAGGAGATTCA (SEQ ID NO: 2285) |
| | | | acetyltransferase) | | GAAAGCTTGTGCCCAAACATGGTGA (SEQ ID NO: 2286) |
| | | | | | AATCTTGTCTATTTGTCATCCAGCT (SEQ ID NO: 2287) |
| | | | | | CATCCAGCTCACCAGTTATCAACTG (SEQ ID NO: 2288) |
| | | | | | GTTATCAACTGACGACCTATCATGT (SEQ ID NO: 2289) |
| | | | | | GACGACCTATCATGTATCTTCTGTA (SEQ ID NO: 2290) |
| | | | | | TATCATGTATCTTCTGTACCCTTAC (SEQ ID NO: 2291) |
| | | | | | AATCCTAGACATCAAATCATTTCAC (SEQ ID NO: 2292) |
| | | | | | AGATGGCCTGTGGTTATCTTGGAAA (SEQ ID NO: 2293) |
| | | | | | GTTGGTTTATTGTTGAATTCCTAGA (SEQ ID NO: 2294) |
| 209061_at | NCOA3 | RP5-1049G16.3, | nuclear receptor | 8202 | ATCTGAGGTGAGTTGGGGGTATCTA (SEQ ID NO: 2295) |
| | | ACTR, AIB-1, | coactivator 3 | | GGCTTGATGTCCTAGAAGTTCTTTG (SEQ ID NO: 2296) |
| | | AIB1, CAGH16, | | | AGTTCTTTGATCCAGAGGTGGGTGC (SEQ ID NO: 2297) |
| | | CTG26, KAT13B, | | | GAGGTGGGTGCAGCTGAAAGTAAAC (SEQ ID NO: 2298) |
| | | MGC141848, | | | TGCCAGTTACATGTATGCCTGCCCA (SEQ ID NO: 2299) |
| | | RAC3, SRC3, | | | CCAGTTCCCTTTTTATTTGCAGAAG (SEQ ID NO: 2300) |
| | | TNRC14, | | | TTTTGTTCACAATTAGGTTCCTAGG (SEQ ID NO: 2301) |
| | | TNRC16, TRAM- | | | AACGAGCAGGGTGTTTTCTCTCTTC (SEQ ID NO: 2302) |
| | | 1, pCIP | | | CAGGAATTGACTTATACTCTTGAGA (SEQ ID NO: 2303) |
| | | | | | ATGGGGGATGGTGAGCTGTGACTGC (SEQ ID NO: 2304) |
| | | | | | ACTGCTTTGCTGACCATTTTGGATG (SEQ ID NO: 2305) |
| 203478_at | NDUFC1 | MGC117464, | NADH | 4717 | GGGGCCCAAGGAATTCGCAAGATGG (SEQ ID NO: 2306) |
| | | MGC126847, | dehydrogenase | | AGGAATTCGCAAGATGGCGCCGTCC (SEQ ID NO: 2307) |
| | | MGC138266 | (ubiquinone) 1, | | GCCCTTCAGTGCGATCAAAGTTCTA (SEQ ID NO: 2308) |
| | | | subcomplex | | GTGCGATCAAAGTTCTACGTGCGAG (SEQ ID NO: 2309) |
| | | | unknown, 1, 6 kDa | | TTCTACGTGCGAGAGCCGCCGAATG (SEQ ID NO: 2310) |
| | | | | | CCGCCGAATGCCAAACCTGACTGGC (SEQ ID NO: 2311) |
| | | | | | ACCTGACTGGCTGAAAGTTGGGTTC (SEQ ID NO: 2312) |
| | | | | | TGAAAGTTGGGTTCACCTTGGGCAC (SEQ ID NO: 2313) |
| | | | | | GTCTTCTTGTGGATCTATCTCATCA (SEQ ID NO: 2314) |
| | | | | | GGATCTATCTCATCAAACAACACAA (SEQ ID NO: 2315) |
| | | | | | CAAGCCAGCATTTGTATTTTGCATC (SEQ ID NO: 2316) |
| 212530_at | NEK7 | | NIMA (never in | 140609 | AAGCTATATCTATTCTAAACCTTAT (SEQ ID NO: 2317) |
| | | | mitosis gene a)- | | TTAGACATTGGTACCAGTTACCCAG (SEQ ID NO: 2318) |
| | | | related kinase 7 | | GGTGGATGAAGGGTATCTCTATATA (SEQ ID NO: 2319) |
| | | | | | AAGTGCTCAACAATGTGCAATGATT (SEQ ID NO: 2320) |
| | | | | | GTAAGATATTACAGCCATTTCATGA (SEQ ID NO: 2321) |
| | | | | | ATGCTTTACCATTCAACATAGTATC (SEQ ID NO: 2322) |
| | | | | | GTATCTATTACAAAACACCTTTCTT (SEQ ID NO: 2323) |
| | | | | | AACACCTTTCTTGTATCCATATACT (SEQ ID NO: 2324) |
| | | | | | GTATCCATATACTTCAGGTGTTGCT (SEQ ID NO: 2325) |
| | | | | | AACCCACAAATGCATACTTACCCTG (SEQ ID NO: 2326) |
| | | | | | TTACCCTGTGCCTCATATTTCAATA (SEQ ID NO: 2327) |
| 204321_at | NEO1 | DKFZp547A066, | neogenin homolog 1 | 4756 | GAAGCCTGTGTCGAGGCAGCTTCCC (SEQ ID NO: 2328) |
| | | DKFZp547B146, | (chicken) | | TCCCTTTGCCTGCTGATATTCTGCA (SEQ ID NO: 2329) |
| | | HsT17534, NGN | | | GCAACCTGCATTTCACTTTGTGGTC (SEQ ID NO: 2330) |
| | | | | | TTTCACTTTGTGGTCAGGCCGTGTC (SEQ ID NO: 2331) |
| | | | | | CGTGTCTTTGTGCTGTGACTGCATC (SEQ ID NO: 2332) |
| | | | | | GTGACTGCATCACCTTTATGGAGTG (SEQ ID NO: 2333) |
| | | | | | GTCCTTGGTGTTCTCCACAAGTGGT (SEQ ID NO: 2334) |
| | | | | | GGTTGACATTTGACTGCTTGTTCCA (SEQ ID NO: 2335) |
| | | | | | TTTGACAGTGTGGGTCGTTCCTGGG (SEQ ID NO: 2336) |
| | | | | | TTCTGAGTCATTGCATCCTCTACCA (SEQ ID NO: 2337) |
| | | | | | TCTACCAGCTGTTAATCCATCACTC (SEQ ID NO: 2338) |
| 206948_at | NEU3 | FLJ12388, SIAL3 | sialidase 3 | 10825 | ATTTTGCCTTAGCTACTGCAGTGGA (SEQ ID NO: 2339) |
| | | | (membrane sialidase) | | TGGCTTGCTTTTGGACCTTGGATGT (SEQ ID NO: 2340) |
| | | | | | TTGGATGTGTCACCTGAACTCTCTG (SEQ ID NO: 2341) |
| | | | | | TCTGGACCTCAGGTTTCCATCTGTA (SEQ ID NO: 2342) |
| | | | | | ATCTTCTCATCCCTAGGACAAGCAT (SEQ ID NO: 2343) |
| | | | | | GCATAGTGCCTGCATGCTTCATGAT (SEQ ID NO: 2344) |
| | | | | | ATGATCAGTAAGTCCTGGCTGCATA (SEQ ID NO: 2345) |
| | | | | | ACCAGGGGACTTACCTTTTCACATG (SEQ ID NO: 2346) |
| | | | | | ACTTACCCCTCATCCGAGTGTGAGG (SEQ ID NO: 2347) |
| | | | | | CAAGCAGGTGTCATGGCAGGAAGGA (SEQ ID NO: 2348) |
| | | | | | GACCAGATCTGTATGATTTGTTCCA (SEQ ID NO: 2349) |
| 209289_at | NFIB | RP11-280O24.2, | nuclear factor I/B | 4781 | TGAACGAATTTATTTTCCCCTCAGT (SEQ ID NO: 2350) |
| | | HMGIC/NFIB, | | | TATGTTGGTTTAGCTGCAGCCTCCT (SEQ ID NO: 2351) |
| | | NFI-RED, NFIB2, | | | GTTGCATCAAGAACCCATAGGGTGT (SEQ ID NO: 2352) |
| | | NFIB3 | | | TGGGAACGCCAGGGTGTAGGATATA (SEQ ID NO: 2353) |
| | | | | | AGGATATAAAAACGCACTCTTGAGA (SEQ ID NO: 2354) |
| | | | | | GTGCTGAAAACTTGCATGGTGCTTT (SEQ ID NO: 2355) |
| | | | | | GGTGCTTTCAGACATTAGCCTTGTT (SEQ ID NO: 2356) |
| | | | | | GACAGATCCATAGTGTGCATGGGCA (SEQ ID NO: 2357) |
| | | | | | GGCAGACACATTTTGCCTCTATGTC (SEQ ID NO: 2358) |
| | | | | | ACTCTTTCCAGTAATCCTAATTTGC (SEQ ID NO: 2359) |
| | | | | | TGCACGAAGATATAATGTCCACATT (SEQ ID NO: 2360) |
| 204109_s_at | NFYA | CBF-A, CBF-B, | nuclear transcription | 4800 | ATGCAGGATCCAAACCAAGCCGATG (SEQ ID NO: 2361) |
| | | FLJ11236, HAP2, | factor Y, alpha | | CTAACCCCACGCCATGTGATGGAGC (SEQ ID NO: 2362) |
| | | NF-YA | | | GCTGATCAAGGTCATGTTTCTCACT (SEQ ID NO: 2363) |
| | | | | | ACATTGATGATCACATTCTGCCCTT (SEQ ID NO: 2364) |
| | | | | | CATTCTGCCCTTTACTACAGGACAG (SEQ ID NO: 2365) |
| | | | | | GAAGTTGCAAGCCTTTGTCTTACTC (SEQ ID NO: 2366) |
| | | | | | GTCTTACTCTTTCAGTCAGGACCTA (SEQ ID NO: 2367) |
| | | | | | GCCAGCCATCTCAGCAGGGAAGAAC (SEQ ID NO: 2368) |
| | | | | | GAACAACCTTCTCAACCAAAACTGC (SEQ ID NO: 2369) |
| | | | | | GGTGGGGGAATCTCACACTGACTAA (SEQ ID NO: 2370) |
| | | | | | GGACGCTTTGTATTCAGAGATGGCT (SEQ ID NO: 2371) |
| 217896_s_at | NIP30 | CDA018, CDA10, | NEFA-interacting | 80011 | GCAGGCAGAGCAGCTACTGACTTTG (SEQ ID NO: 2372) |
| | | FLJ21799, | nuclear protein NIP30 | | AGTACAGTGACACTCTGGTGCCCAT (SEQ ID NO: 2373) |
| | | MGC74898 | | | CATTGGCAGATGGCGACTTCCCTGC (SEQ ID NO: 2374) |
| | | | | | TTCCTCCTTTTCAGAACTACTCTGT (SEQ ID NO: 2375) |
| | | | | | ACTACTCTGTGCTAATTGTTCTGCC (SEQ ID NO: 2376) |
| | | | | | TGCCAGTATGGGCGCATCAGCTCCA (SEQ ID NO: 2377) |
| | | | | | AAACTTTGTAGGCACCTTCTGCTTC (SEQ ID NO: 2378) |
| | | | | | CTCTGCTTCATTGTTCCTGTGATAG (SEQ ID NO: 2379) |
| | | | | | GTTCCTGTGATAGTCCTGTTGTTAT (SEQ ID NO: 2380) |
| | | | | | GGAGGCAGGCCAGGACATCAAAGTC (SEQ ID NO: 2381) |
| | | | | | GTATCTCATGGCCTCTTGATGTGGA (SEQ ID NO: 2382) |
| 201591_s_at | NISCH | 1200007D05Rik, | nischarin | 11188 | TTGGGAGTGAGAATGCCGGGCCCCT (SEQ ID NO: 2383) |
| | | 3202002H23Rik, | | | CAGGGCTGTCGGTGTGCTGTCAGCC (SEQ ID NO: 2384) |
| | | AW494485, I-1 | | | ACACCAGTGTCGTGTCTGCTGTTGT (SEQ ID NO: 2385) |
| | | | | | GGGACCGTTGTTAACACGTGACACT (SEQ ID NO: 2386) |
| | | | | | GTGACACTGTGGGTCTGACTTTCTC (SEQ ID NO: 2387) |
| | | | | | ACGTCCTTTCCTGAAGTGTCGAGTC (SEQ ID NO: 2388) |
| | | | | | AGAATGCACATTGGAAGCTCCCACC (SEQ ID NO: 2389) |
| | | | | | CCCACCCCATATTGTTCTTCAAAGT (SEQ ID NO: 2390) |
| | | | | | TCAAAGTGGAGGTCTCCCCTGATCC (SEQ ID NO: 2391) |
| | | | | | CAGCACCAAGCGTGATTCCTGCTGC (SEQ ID NO: 2392) |
| | | | | | CTGCCTGTATTCTCTATTCCAATAA (SEQ ID NO: 2393) |
| 221691_x_at | NPM1 | B23, | nucleophosmin | 4869 | GGGGCTTTGAAATAACACCACCAGT (SEQ ID NO: 2394) |
| | | MGC104254, | (nucleolar | | CACCACCAGTGGTCTTAAGGTTGAA (SEQ ID NO: 2395) |
| | | NPM | phosphoprotein B23, | | GAAGTGTGGTTCAGGGCCAGTGCAT (SEQ ID NO: 2396) |
| | | | numatrin) | | GGGCCAGTGCATATTAGTGGACAGC (SEQ ID NO: 2397) |
| | | | | | TATATCTGGAAAGCGGTCTGCCCCT (SEQ ID NO: 2398) |
| | | | | | TCTGCCCCTGGAGGTGGTAGCAAGG (SEQ ID NO: 2399) |
| | | | | | GAGATACTCCAGCCAAAAATGCACA (SEQ ID NO: 2400) |
| | | | | | GACTCAAAACCATCATCAACACCAA (SEQ ID NO: 2401) |
| | | | | | ACACCAAAAGGACCTAGTTCTGTAG (SEQ ID NO: 2402) |
| | | | | | AAAGCGCATTGAACAGTCCTGGGCA (SEQ ID NO: 2403) |
| | | | | | AACAGTCCTGGGCACTACATGTAAA (SEQ ID NO: 2404) |
| 214066_x_at | NPR2 | AMDM, ANPRB, | natriuretic peptide | 4882 | AGAGCGGAAAGGACCTCCTGGACTC (SEQ ID NO: 2405) |
| | | GUC2B, | receptor B/guanylate | | CCAAGTCAGATAGTCTTCTGCTGCT (SEQ ID NO: 2406) |
| | | GUCY2B, NPRB, | cyclase B | | CAGATAGTCTTCTGCTGCTGGTACC (SEQ ID NO: 2407) |
| | | NPRBi | (atrionatriuretic | | TGCTGGTACCTGGGTGGGCAATGGC (SEQ ID NO: 2408) |
| | | | peptide receptor B) | | GGTGGGCAATGGCCACCATGTCTGC (SEQ ID NO: 2409) |
| | | | | | GAAATGGACATTTTCATATGCAATG (SEQ ID NO: 2410) |
| | | | | | ATATGCAATGGAAAACAGCCACAAA (SEQ ID NO: 2411) |
| | | | | | GCCACAAAAAAACCTACCTTATATG (SEQ ID NO: 2412) |
| | | | | | TATATGGAAGTTGTAGCCCTCTGCA (SEQ ID NO: 2413) |
| | | | | | CTTCCTCCCTACTTTCTGTAAATAT (SEQ ID NO: 2414) |
| | | | | | TCTGTAAATATCTGTATCTAAACCA (SEQ ID NO: 2415) |
| 219789_at | NPR3 | ANPRC, | natriuretic peptide | 4883 | TTATGATTAATCACCATCTGCCTCC (SEQ ID NO: 2416) |
| | | GUCY2B, NPRC | receptor C/guanylate | | CCAGGCCTTTCATCTCATGACAAAC (SEQ ID NO: 2417) |
| | | | cyclase C | | ATCGTGTCACTCTGTTAAATGTTCA (SEQ ID NO: 2418) |
| | | | (atrionatriuretic | | TGTTCATACTGTTTCAAGCCCATAT (SEQ ID NO: 2419) |
| | | | peptide receptor C) | | TGTTGTCTCCATATCTTGATGGCTT (SEQ ID NO: 2420) |
| | | | | | GGCTTTTGGGAGCATTTCACACAAG (SEQ ID NO: 2421) |
| | | | | | CAGGTTTGTGGTTGAGGACTTCTCT (SEQ ID NO: 2422) |
| | | | | | GGACTTCTCTGTCCGATGTCTACAT (SEQ ID NO: 2423) |
| | | | | | GTCTACATTCAGGTTCTGACTTCAT (SEQ ID NO: 2424) |
| | | | | | TCCCTGTCTTTTTCAGTGTCTCATA (SEQ ID NO: 2425) |
| | | | | | GTCTCATAAACGCTACTCTGGATTG (SEQ ID NO: 2426) |
| 219214_s_at | NT5C | DNT, DNT1, | 5′,3′-nucleotidase, | 30833 | GAAGTACCACCACTGTGTGGGTGAG (SEQ ID NO: 2427) |
| | | P5N2, PN-I, PN- | cytosolic | | AGTACCGCTGGGTGGAGCAGCACCT (SEQ ID NO: 2428) |
| | | II, UMPH2, cdN, | | | GTAGAACGAATTATCCTGACAAGGG (SEQ ID NO: 2429) |
| | | dNT-1 | | | TCTTGGGGGACCTGCTCATTGATGA (SEQ ID NO: 2430) |
| | | | | | GACAAGGACACAGTTCGAGGCCAGG (SEQ ID NO: 2431) |
| | | | | | GCCAGGAGGAGACCCCAAGCTGGGA (SEQ ID NO: 2432) |
| | | | | | CAAGCTGGGAGCACATCTTGTTCAC (SEQ ID NO: 2433) |
| | | | | | GATAGCAAGCGCGGAGCTGCGCAGC (SEQ ID NO: 2434) |
| | | | | | GCTGCGCAGCGGGAATGAGCGGGGA (SEQ ID NO: 2435) |
| | | | | | GGAATGAGCGGGGATGCCGCGGGCA (SEQ ID NO: 2436) |
| | | | | | AGGAAGGGCAGGCCCACAGGGGCCA (SEQ ID NO: 2437) |
| 205136_s_at | NUFIP1 | MGC95323 | nuclear fragile X | 26747 | AGGGATGTTGTTGATCCCCACACTG (SEQ ID NO: 2438) |
| | | | mental retardation | | ATCCCCACACTGCCTTAAGGTATAT (SEQ ID NO: 2439) |
| | | | protein interacting | | TCCCCTGGCTGTATGTCTGACAGTC (SEQ ID NO: 2440) |
| | | | protein 1 | | ATAGCCTGTATGGTTTCTACTTTAT (SEQ ID NO: 2441) |
| | | | | | GGGTGGGTAGAGGGGTGCTTTGCCT (SEQ ID NO: 2442) |
| | | | | | TGTTAATTGTTCAAACTTTCTGCCT (SEQ ID NO: 2443) |
| | | | | | ACATTCCTATCTTATTGTTCGTTTA (SEQ ID NO: 2444) |
| | | | | | GACTATTGGTGAGTTGTGCCTTTTA (SEQ ID NO: 2445) |
| | | | | | TTACTTTTTCTCATATCCTCTATAA (SEQ ID NO: 2446) |
| | | | | | ATAATTGACTTCTATTTTCCTTAAT (SEQ ID NO: 2447) |
| | | | | | TATTTTCCTTAATCAAACCAGCTCT (SEQ ID NO: 2448) |
| 209073_s_at | NUMB | S171 | numb homolog | 8650 | TTGTTTTTCAGGCCATTCACCTGCC (SEQ ID NO: 2449) |
| | | | (Drosophila) | | CCTGTCAGTAGTGGCCTGTATTAGA (SEQ ID NO: 2450) |
| | | | | | AAGAGTGGTTTGTGCTCAGGCTGGG (SEQ ID NO: 2451) |
| | | | | | GGAACAGAGAGGCACGCTATGCTGC (SEQ ID NO: 2452) |
| | | | | | CGCTATGCTGCCAGAATTCCCAGGA (SEQ ID NO: 2453) |
| | | | | | TTCCCAGGAGGGCATATCAGCAACT (SEQ ID NO: 2454) |
| | | | | | TTGAGCTTCCATTTTGAGTAACAGA (SEQ ID NO: 2455) |
| | | | | | TTTATCATTGATGCCTACTGAAATA (SEQ ID NO: 2456) |
| | | | | | AGACAGGATCCCTAGCTTGTTTTCT (SEQ ID NO: 2457) |
| | | | | | GCAACAACAATCATGCTTATGACCA (SEQ ID NO: 2458) |
| | | | | | GACCAATACAGTCACTAGGTTGTAG (SEQ ID NO: 2459) |
| 207563_s_at | OGT | FLJ23071, | O-linked N- | 8473 | AGCGTGTTCCCAATAGTGTACTCTG (SEQ ID NO: 2460) |
| | | HRNT1, | acetylglucosamine | | TACTCTGGCTGTTGCGTTTTCCAGC (SEQ ID NO: 2461) |
| | | MGC22921, O- | (GlcNAc) transferase | | GCCCCAGAACCGTATCATTTTTTCA (SEQ ID NO: 2462) |
| | | GLCNAC | (UDP-N- | | GAGGAACACGTCAGGAGAGGCCAGC (SEQ ID NO: 2463) |
| | | | acetylglucosamine:polypeptide- | | GGACACTCCACTCTGTAATGGGCAC (SEQ ID NO: 2464) |
| | | | N- | | GATGGATGTCCTCTGGGCAGGGACC (SEQ ID NO: 2465) |
| | | | acetylglucosaminyl | | ACCCCCATGGTGACTATGCCAGGAG (SEQ ID NO: 2466) |
| | | | transferase) | | AGGAGAGACTCTTGCTTCTCGAGTT (SEQ ID NO: 2467) |
| | | | | | ATCCCAGCTCACTTGCTTAGGTTGT (SEQ ID NO: 2468) |
| | | | | | GAGCGGCTCTATCTACAGATGTGGG (SEQ ID NO: 2469) |
| | | | | | TGCAGCTGGCAACAAACCTGACCAC (SEQ ID NO: 2470) |
| 212307_s_at | OGT | FLJ23071, | O-linked N- | 8473 | GATAACATACTTCTTACTTGTCTGT (SEQ ID NO: 2471) |
| | | HRNT1, | acetylglucosamine | | ACTTGTCTGTACAGTACCTTGTTGC (SEQ ID NO: 2472) |
| | | MGC22921, O- | (GlcNAc) transferase | | AGACTTGCTTCCTGCATGGTAGGGA (SEQ ID NO: 2473) |
| | | GLCNAC | (UDP-N- | | AACTGCTTTTCCACAAGGAATCTCC (SEQ ID NO: 2474) |
| | | | acetylglucosamine:polypeptide- | | GAATTTTGCGGCGACCAGATGGTGC (SEQ ID NO: 2475) |
| | | | N- | | AGGTCTGGAAGGTCTGATCTCCCTT (SEQ ID NO: 2476) |
| | | | acetylglucosaminyl | | TCCCTTGGTCTTCCATGGGATGGTT (SEQ ID NO: 2477) |
| | | | transferase) | | GGAGATATAGATTGTCCGGCCGCTT (SEQ ID NO: 2478) |
| | | | | | GGCCGCTTTGTGATTCCATGGATTG (SEQ ID NO: 2479) |
| | | | | | ATGTTACTCCCTGTTTTAGTTTCTG (SEQ ID NO: 2480) |
| | | | | | GGGACTTTGCTGGTGTAGTCTTTTT (SEQ ID NO: 2481) |
| 212213_x_at | OPA1 | FLJ12460, | optic atrophy 1 | 4976 | TTATGATATTGCCCTCTTTGTATTC (SEQ ID NO: 2482) |
| | | KIAA0567, | (autosomal dominant) | | TGTATTCCCATTTTCCACAGTTTTT (SEQ ID NO: 2483) |
| | | MGM1, NPG, | | | GTTTTTTCCGCAGACTTCTTTCTGC (SEQ ID NO: 2484) |
| | | NTG, largeG | | | AATTATTCAGCCTCCAAATGCAAAT (SEQ ID NO: 2485) |
| | | | | | ATGGCAGAGAGTGGTGCTTCCCAGC (SEQ ID NO: 2486) |
| | | | | | TGCTTCCCAGCCTCACAATGTGGGA (SEQ ID NO: 2487) |
| | | | | | TAATGGTTTTTCTCTCAGTTCTCTA (SEQ ID NO: 2488) |
| | | | | | AGTTCTCTAAGCTGGTCTATGTTAT (SEQ ID NO: 2489) |
| | | | | | AATATGCTTACCTTTTGAATGATCA (SEQ ID NO: 2490) |
| | | | | | AACTTACCTTGTTTTCACTTGTGCA (SEQ ID NO: 2491) |
| | | | | | ACTTATATTTTGCCTGAGCTTGATC (SEQ ID NO: 2492) |
| 222327_x_at | OR7E156P | | olfactory receptor, | 283491 | GGTACCTCCGTCGGCAGATTTGAGC (SEQ ID NO: 2493) |
| | | | family 7, subfamily E, | | AGATTTGAGCTTTCTCCTTGGACAC (SEQ ID NO: 2494) |
| | | | member 156 | | CCTTGGACACCTAATACCCACAGTC (SEQ ID NO: 2495) |
| | | | pseudogene | | TGCGTTCTGACACCTGTCGGATAGT (SEQ ID NO: 2496) |
| | | | | | GTCGGCAGATTTGAGCTTTCTTCTT (SEQ ID NO: 2497) |
| | | | | | TTTCTTCTTGGACACCTATACCCAC (SEQ ID NO: 2498) |
| | | | | | CAGTCCTCCAGTGTTTTAGACGCCC (SEQ ID NO: 2499) |
| | | | | | GCCCAGCTGCACAACTTGATTGCCT (SEQ ID NO: 2500) |
| | | | | | GATTGCCTTACAAATGACCTGCTTC (SEQ ID NO: 2501) |
| | | | | | ATGACCTGCTTCCAGGATGCGGAAA (SEQ ID NO: 2502) |
| | | | | | AATTCCTAATTTCTTCTGTGACCCT (SEQ ID NO: 2503) |
| 219073_s_at | OSBPL10 | FLJ20363, | oxysterol binding | 114884 | TGGTCACAGCGCTAGTCATTCATTT (SEQ ID NO: 2504) |
| | | ORP10, OSBP9 | protein-like 10 | | TGAAAACATGCACAACCACAGCCTC (SEQ ID NO: 2505) |
| | | | | | CCACAGCCTCAATCTTGTATTTAGT (SEQ ID NO: 2506) |
| | | | | | TTTCCTGTGGATTATTTTTTCCTCA (SEQ ID NO: 2507) |
| | | | | | GACTAAAACATACATAACGCCTGCA (SEQ ID NO: 2508) |
| | | | | | AACGCCTGCATAAATATTCCATGGT (SEQ ID NO: 2509) |
| | | | | | TTTCAGTTTTTAGCCTTTACACATG (SEQ ID NO: 2510) |
| | | | | | AATATCTGGTTTTTGCTGAATGCTT (SEQ ID NO: 2511) |
| | | | | | TTACTGTGCCACTCCAATATTTATC (SEQ ID NO: 2512) |
| | | | | | GCTTTCTTAAGACCAGTTGTTCACA (SEQ ID NO: 2513) |
| | | | | | GACCAGTTGTTCACACTTTGTAGAT (SEQ ID NO: 2514) |
| 209626_s_at | OSBPL3 | 1200014M06Rik, | oxysterol binding | 26031 | ATAGCTTCACACAGTTTGCGCTGGA (SEQ ID NO: 2515) |
| | | 6720421I08Rik, | protein-like 3 | | CAAAGTCTTTATTGCCACCTACTGA (SEQ ID NO: 2516) |
| | | A530055M08, | | | GACACTCGATTTAGGCCAGACCAGA (SEQ ID NO: 2517) |
| | | MGC102333, | | | GAGAGAAAGGCGGCGGGTCTTAGAA (SEQ ID NO: 2518) |
| | | ORP3, OSBP3 | | | GGAAATCCGACGATGACTCTTGGGT (SEQ ID NO: 2519) |
| | | | | | GAGCAACGGCACCTATTTGGAACTT (SEQ ID NO: 2520) |
| | | | | | ACTGGACCATCCTGTCTTATGGTGA (SEQ ID NO: 2521) |
| | | | | | ATAACATTAGTGTATTTCTCCTGTG (SEQ ID NO: 2522) |
| | | | | | GATGATCACTTGTGCTTAGCTTAGC (SEQ ID NO: 2523) |
| | | | | | TCTTTAAGTCTATATTTTCCTCAGT (SEQ ID NO: 2524) |
| | | | | | TTTCCTCAGTGCGTTTCTTTACAAT (SEQ ID NO: 2525) |
| 209627_s_at | OSBPL3 | 1200014M06Rik, | oxysterol binding | 26031 | GTTATGCCTTCTGGCTACTACAGAA (SEQ ID NO: 2526) |
| | | 6720421I08Rik, | protein-like 3 | | ACAGAATCTCCGCAGACCTTTTAGT (SEQ ID NO: 2527) |
| | | A530055M08, | | | GAAGGTGTCCTTATGGTCTTAGGAA (SEQ ID NO: 2528) |
| | | MGC102333, | | | ATAGCTATTTTCATTTGCTCTATTT (SEQ ID NO: 2529) |
| | | ORP3, OSBP3 | | | TGCTCTATTTTCCTCTGCATTGTAC (SEQ ID NO: 2530) |
| | | | | | GCATTGTACTAATCTGTTACTCAGT (SEQ ID NO: 2531) |
| | | | | | GTTACTCAGTGTTAGTCTTCATTTT (SEQ ID NO: 2532) |
| | | | | | GTGTTGGAAACCTGCGTCATGTAAA (SEQ ID NO: 2533) |
| | | | | | AATCTTCCACTTAACCACTGAAAAT (SEQ ID NO: 2534) |
| | | | | | AGCTCCTATTAATTTGCTTATCCCC (SEQ ID NO: 2535) |
| | | | | | TATCCCCCTCATGTAGCTAGTTGAA (SEQ ID NO: 2536) |
| 200656_s_at | P4HB | PDI | procollagen-proline, | 5034 | AGTGCCGACAGGACGGTCATTGATT (SEQ ID NO: 2537) |
| | | | 2-oxoglutarate 4- | | GGACGGTCATTGATTACAACGGGGA (SEQ ID NO: 2538) |
| | | | dioxygenase (proline | | GATTACAACGGGGAACGCACGCTGG (SEQ ID NO: 2539) |
| | | | 4-hydroxylase), beta | | TACAACGGGGAACGCACGCTGGATG (SEQ ID NO: 2540) |
| | | | polypeptide | | GGGAACGCACGCTGGATGGTTTTAA (SEQ ID NO: 2541) |
| | | | | | GAAGCAGAGGAGCCAGACATGGAGG (SEQ ID NO: 2542) |
| | | | | | AAGATGAACTGTAATACGCAAAGCC (SEQ ID NO: 2543) |
| | | | | | GTTTCCTGAAACCATGATGTACTTT (SEQ ID NO: 2544) |
| | | | | | GTACTTTTTCATACATGAGTCTGTC (SEQ ID NO: 2545) |
| | | | | | CATACATGAGTCTGTCCAGAGTGCT (SEQ ID NO: 2546) |
| | | | | | TGCTTGCTACCGTGTTCGGAGTCTC (SEQ ID NO: 2547) |
| 201013_s_at | PAICS | ADE2, ADE2H1, | phosphoribosylaminoimidazole | 10606 | AACTGGCATTACTGACTCCCAGCTA (SEQ ID NO: 2548) |
| | | AIRC, | carboxylase, | | CCAGCTATATTTCTCCAGACTTGCA (SEQ ID NO: 2549) |
| | | DKFZp781N1372, | phosphoribosylaminoimidazole | | AATTGCTGATGTGACCCATGAAGGG (SEQ ID NO: 2550) |
| | | MGC1343, | succinocarboxamide | | TTTTCCTCGTAATTTTGGACTGCCA (SEQ ID NO: 2551) |
| | | MGC5024, PAIS | synthetase | | TGGACTGCCACACATTGGTACCTTT (SEQ ID NO: 2552) |
| | | | | | ACATTGGTACCTTTAGTTCTCTGAA (SEQ ID NO: 2553) |
| | | | | | TTCTCTGAAGGCCCACGTTTTTATC (SEQ ID NO: 2554) |
| | | | | | GTAGAGCTTTATGTTCGCTGTCCAT (SEQ ID NO: 2555) |
| | | | | | GCTGTCCATGAAACCTTCTGTAACC (SEQ ID NO: 2556) |
| | | | | | TGACTACAAGTAGTTCTTTCTCTAT (SEQ ID NO: 2557) |
| | | | | | GCTATGCTTAGCTATCTGTCAGAGA (SEQ ID NO: 2558) |
| 212958_x_at | PAM | PAL, PHM | peptidylglycine alpha- | 5066 | AATTTCCACAGATACTTCCCTTAGA (SEQ ID NO: 2559) |
| | | | amidating | | TGAGCGAGGCCTTGTCAATTTTAAG (SEQ ID NO: 2560) |
| | | | monooxygenase | | TAGGAAGGACCACAACATGACCCGT (SEQ ID NO: 2561) |
| | | | | | TACACACTTTATTTACTTCGTTTTG (SEQ ID NO: 2562) |
| | | | | | GTTGGCTTCTGTTTCTAGTTGAGGA (SEQ ID NO: 2563) |
| | | | | | TCCTCTTTTTCCATCATAATTCTAA (SEQ ID NO: 2564) |
| | | | | | GATTTGCCCATTTACACTTTTGAGA (SEQ ID NO: 2565) |
| | | | | | GTAAATAACCCCATTCTTTGCTTGA (SEQ ID NO: 2566) |
| | | | | | GTATTTTCCCAATAGCACTTTCATT (SEQ ID NO: 2567) |
| | | | | | ATTGCCAGTGTCTTTCTTTGGTGCC (SEQ ID NO: 2568) |
| | | | | | TTCAGCATTCTTAGCCTGTGGCAAT (SEQ ID NO: 2569) |
| 219148_at | PBK | SPK; TOPK; | PDZ binding kinase | 55872 | AGCATACTATGCAGCGTTGGGAACT (SEQ ID NO: 2570) |
| | | Nori-3; FLJ14385 | | | CAGCGTTGGGAACTAGGCCACCTAT (SEQ ID NO: 2571) |
| | | | | | TGAACTCTTCTCTGTATGCACTAAT (SEQ ID NO: 2572) |
| | | | | | AGACCCTAAAGATCGTCCTTCTGCT (SEQ ID NO: 2573) |
| | | | | | ATGTCTAGTGATCATCTCAGCTGAA (SEQ ID NO: 2574) |
| | | | | | GTGTGGCTTGCGTAAATAACTGTTT (SEQ ID NO: 2575) |
| | | | | | GAGGACCATAGTTTCTTGTTAACAT (SEQ ID NO: 2576) |
| | | | | | AAGCACTTGGAATTGTACTGGGTTT (SEQ ID NO: 2577) |
| | | | | | GTACTTTGATACTGCTCATGCTGAC (SEQ ID NO: 2578) |
| | | | | | TGCTCATGCTGACTTAAAACACTAG (SEQ ID NO: 2579) |
| | | | | | GGATCTACTGACATTAGCACTTTGT (SEQ ID NO: 2580) |
| 204476_s_at | PC | PCB | pyruvate carboxylase | 5091 | GGTGACCTCACCCATGGAGGGTACT (SEQ ID NO: 2581) |
| | | | | | GGTACTGTCCGCAAGGTTCATGTGA (SEQ ID NO: 2582) |
| | | | | | TGGAAGGTGACGACCTCATCCTGGA (SEQ ID NO: 2583) |
| | | | | | GGAGATCGAGTGATCTTGCCCCAGA (SEQ ID NO: 2584) |
| | | | | | TCAACAGAAGCTGTGCTGCCACGGC (SEQ ID NO: 2585) |
| | | | | | CAGTGCCCGAGGCCAGGAAGGCCGG (SEQ ID NO: 2586) |
| | | | | | GACACCGCCTGCGGTGGTTCATTCC (SEQ ID NO: 2587) |
| | | | | | GACAGCTGCTTACATGTTCATCTCT (SEQ ID NO: 2588) |
| | | | | | ATCTCTTGCCAAATAAGGGTCCCCT (SEQ ID NO: 2589) |
| | | | | | GGGTCAGGTGGTCCTAGGACCCAGG (SEQ ID NO: 2590) |
| | | | | | AGATCTAAGATGTCCCAGGTCCTGG (SEQ ID NO: 2591) |
| 214239_x_at | PCGF2 | MEL-18, | polycomb group ring | 7703 | AGATTTCCTCAAGTGTGCATAGATC (SEQ ID NO: 2592) |
| | | MGC10545, | finger 2 | | GTGTGCATAGATCTGAGAGGAGTCG (SEQ ID NO: 2593) |
| | | RNF110, ZNF144 | | | GTCGAGACTCGAGATTCCATCATAG (SEQ ID NO: 2594) |
| | | | | | GAGATTCCATCATAGCGTAGGTGTG (SEQ ID NO: 2595) |
| | | | | | TAGCGTAGGTGTGTGGGGTTGGGAG (SEQ ID NO: 2596) |
| | | | | | CCCTGATGGGCTTGTCTGTGTTTGC (SEQ ID NO: 2597) |
| | | | | | CTGTGTCTGAGGTCCTGTGACTGTA (SEQ ID NO: 2598) |
| | | | | | TCTGAGGTCCTGTGACTGTACCCTC (SEQ ID NO: 2599) |
| | | | | | TCCTTTGCCCTGGGACATCTGTATC (SEQ ID NO: 2600) |
| | | | | | CATCTGTATCTCTTGGCTTTGTAAT (SEQ ID NO: 2601) |
| | | | | | GGCTTTGTAATAAATGCTGCATACT (SEQ ID NO: 2602) |
| 210836_x_at | PDE4D | DPDE3, | phosphodiesterase | 5144 | GACATAGCACGAATCTGTTACCAGT (SEQ ID NO: 2603) |
| | | HSPDE4D, | 4D, cAMP-specific | | AATTTTCTAATTTCAAGTCTTCCTG (SEQ ID NO: 2604) |
| | | PDE4DN2, | (phosphodiesterase | | GAATAGTGTGGTTCAGTGAGCTGCA (SEQ ID NO: 2605) |
| | | STRK1 | E3 dunce homolog, | | CTGCACTGACCTCTACATTTTGTAT (SEQ ID NO: 2606) |
| | | | Drosophila) | | ATGTTCAGAACTTCATCTGCCACTG (SEQ ID NO: 2607) |
| | | | | | ATCTGCCACTGGTTATTTTTTTCTA (SEQ ID NO: 2608) |
| | | | | | GAATTTTACTTGCACCTTATAGTTC (SEQ ID NO: 2609) |
| | | | | | TGCAGGAAACCCTTTTTGTAAGTCT (SEQ ID NO: 2610) |
| | | | | | TTTGCATTTTGTTTCACTCTTTCCA (SEQ ID NO: 2611) |
| | | | | | GATAAGCAGAGTTGCTCTTCACCAG (SEQ ID NO: 2612) |
| | | | | | TGCTCTTCACCAGTGTTTTTCTTCA (SEQ ID NO: 2613) |
| 205463_s_at | PDGFA | PDGF-A, PDGF1 | platelet-derived | 5154 | TACACCGATAACACACAGGCTGCTG (SEQ ID NO: 2614) |
| | | | growth factor alpha | | CAGGCTGCTGTAACTGTCAGGACAG (SEQ ID NO: 2615) |
| | | | polypeptide | | GACAGTGCGACGGTATTTTTCCTAG (SEQ ID NO: 2616) |
| | | | | | ATGGTATACCTACCTATGCATCATT (SEQ ID NO: 2617) |
| | | | | | AATGTTTCTGGCTTTGTGTTTCTCC (SEQ ID NO: 2618) |
| | | | | | ATGCGTCAACCAATCGTACGCCGTC (SEQ ID NO: 2619) |
| | | | | | CCCAGAGCCCGTTTGTGGCTGAGTG (SEQ ID NO: 2620) |
| | | | | | GTGGCTGAGTGACAACTTGTTCCCC (SEQ ID NO: 2621) |
| | | | | | CCCGCAGTGCACACCTAGAATGCTG (SEQ ID NO: 2622) |
| | | | | | GGCACGTGAGATGCATTGCCGCTTC (SEQ ID NO: 2623) |
| | | | | | GCTGCCAGTGTTTGGACAGAACCCA (SEQ ID NO: 2624) |
| 205353_s_at | PEBP1 | HCNP, PBP, | phosphatidylethanolamine | 5037 | TCACTCACTCTGATTTATGTTTTGA (SEQ ID NO: 2625) |
| | | PEBP, RKIP | binding protein 1 | | TTGGGGGGTATTTTGGTACTGTGAT (SEQ ID NO: 2626) |
| | | | | | ATTTTGGTACTGTGATGGGGTCATC (SEQ ID NO: 2627) |
| | | | | | AAGACCAGGTCTACAGTGATAGAGC (SEQ ID NO: 2628) |
| | | | | | GATCCTGAGTCCAGAATGGTACACA (SEQ ID NO: 2629) |
| | | | | | GTGATGTCACTCACCTAGACAACCA (SEQ ID NO: 2630) |
| | | | | | CTCACCTAGACAACCAGAGGCTGGC (SEQ ID NO: 2631) |
| | | | | | CCAGAGGCTGGCATTGAGGCTAACC (SEQ ID NO: 2632) |
| | | | | | GGCATTGAGGCTAACCTCCAACACA (SEQ ID NO: 2633) |
| | | | | | TAACCTCCAACACAGTGCATCTCAG (SEQ ID NO: 2634) |
| | | | | | TGCATCTCAGATGCCTCAGTAGGCA (SEQ ID NO: 2635) |
| 211941_s_at | PEBP1 | HCNP, PBP, | phosphatidylethanolamine | 5037 | CACAGTCCTCTCCGATTATGTGGGC (SEQ ID NO: 2636) |
| | | PEBP, RKIP | binding protein 1 | | CACCGCTATGTCTGGCTGGTTTACG (SEQ ID NO: 2637) |
| | | | | | GGCCGCTAAAGTGTGACGAGCCCAT (SEQ ID NO: 2638) |
| | | | | | CCATCCTCAGCAACCGATCTGGAGA (SEQ ID NO: 2639) |
| | | | | | CAAATTCAAGGTGGCGTCCTTCCGT (SEQ ID NO: 2640) |
| | | | | | CTGGCACGTGTTACCAGGCCGAGTG (SEQ ID NO: 2641) |
| | | | | | GGATGACTATGTGCCCAAACTGTAC (SEQ ID NO: 2642) |
| | | | | | TGGGGACCTGAACTGTCCTGGAGGC (SEQ ID NO: 2643) |
| | | | | | TTCCCCAGTTCAGTGTTGCATGTAT (SEQ ID NO: 2644) |
| | | | | | CCCCCCTTGGCATGGGTGAGACCTG (SEQ ID NO: 2645) |
| | | | | | CCTGCTGCCTGGCCTTTATAATTTT (SEQ ID NO: 2646) |
| 217923_at | PEF1 | PEF1A, PEFLIN | penta-EF-hand | 553115 | GGGGCTGAGGCCACACAGATAGGAG (SEQ ID NO: 2647) |
| | | | domain containing 1 | | GAATGTCCTGATGGCCATGAGCAGT (SEQ ID NO: 2648) |
| | | | | | CATGAGCAGTTGAGTGGCACAGCCT (SEQ ID NO: 2649) |
| | | | | | GGCACCAGGAGCAGGTCCTTGTAAT (SEQ ID NO: 2650) |
| | | | | | AGTTAGTGTCCAGTCAGCTGAGCTC (SEQ ID NO: 2651) |
| | | | | | CAGTGGTGAGTGTTCATCGGCCTGT (SEQ ID NO: 2652) |
| | | | | | ATCGGCCTGTTACCGTTAGTACCTG (SEQ ID NO: 2653) |
| | | | | | ACCAGGCCATCCTGTCAAACGAGCC (SEQ ID NO: 2654) |
| | | | | | GATCTGTCTATGGGACCAGTGGCTT (SEQ ID NO: 2655) |
| | | | | | TCTGCCACACCCATAAATCCTTGTG (SEQ ID NO: 2656) |
| | | | | | TTGTGTGTTAACTTCTAGCTGCCTG (SEQ ID NO: 2657) |
| 200634_at | PFN1 | | profilin 1 | 5216 | GAAAACGTTCGTCAACATCACGCCA (SEQ ID NO: 2658) |
| | | | | | ATCACGCCAGCTGAGGTGGGTGTCC (SEQ ID NO: 2659) |
| | | | | | TCCTGGTTGGCAAAGACCGGTCAAG (SEQ ID NO: 2660) |
| | | | | | GACCGGTCAAGTTTTTACGTGAATG (SEQ ID NO: 2661) |
| | | | | | AATGTTCGGTGATCCGGGACTCACT (SEQ ID NO: 2662) |
| | | | | | GAATTTAGCATGGATCTTCGTACCA (SEQ ID NO: 2663) |
| | | | | | TTCGTACCAAGAGCACCGGTGGGGC (SEQ ID NO: 2664) |
| | | | | | ACCTTCAATGTCACTGTCACCAAGA (SEQ ID NO: 2665) |
| | | | | | CTGACAAGACGCTAGTCCTGCTGAT (SEQ ID NO: 2666) |
| | | | | | GTGTCCACGGTGGTTTGATCAACAA (SEQ ID NO: 2667) |
| | | | | | CCCTTATTGCTGCCAAAACCACATG (SEQ ID NO: 2668) |
| 206726_at | PGDS | | prostaglandin D2 | 27306 | CTTACTCTTCACCAGAGCCTAGCAA (SEQ ID NO: 2669) |
| | | | synthase, | | TGAGCTGCTCACGTATAATGCGCCT (SEQ ID NO: 2670) |
| | | | hematopoietic | | GTAACTTGGGCAGACTTCTACTGGG (SEQ ID NO: 2671) |
| | | | | | GGAGATTTGCAGTACCACACTTTTG (SEQ ID NO: 2672) |
| | | | | | AGCCTGACCTGTTAGACAACCATCC (SEQ ID NO: 2673) |
| | | | | | CCATCCAAGGCTGGTGACTTTACGG (SEQ ID NO: 2674) |
| | | | | | TTCCTGCCGTCGCTAACTGGATAAA (SEQ ID NO: 2675) |
| | | | | | CAAACCAAACTCTAGCTGATCCATG (SEQ ID NO: 2676) |
| | | | | | GATCCATGTTGCCTTCAAGTTTGTT (SEQ ID NO: 2677) |
| | | | | | TCAGCCTGCCAGATAATCCACATGC (SEQ ID NO: 2678) |
| | | | | | CCCAGCTCCACTAAGATTTTCACTT (SEQ ID NO: 2679) |
| 215832_x_at | PICALM | CALM, CLTH, | phosphatidylinositol | 8301 | GAGAACCCGCTGGTTTATTGCGACG (SEQ ID NO: 2680) |
| | | LAP | binding clathrin | | CGCGGTGCATCAAGCTTGCTATGGC (SEQ ID NO: 2681) |
| | | | assembly protein | | ATGGCATTGTTCAAGTACCCACTGG (SEQ ID NO: 2682) |
| | | | | | GTACCCACTGGACCGTGGTTTTGCA (SEQ ID NO: 2683) |
| | | | | | GAGAGAGCAGCCAGAGTGGCACCCC (SEQ ID NO: 2684) |
| | | | | | CCTATCCTGCTACTACACCAACAGG (SEQ ID NO: 2685) |
| | | | | | GTGTTCCTGTAATGACGCAACCAAC (SEQ ID NO: 2686) |
| | | | | | CCCCTTTGGCCCTGTATCAGGAGCA (SEQ ID NO: 2687) |
| | | | | | AGCAGCAAAATCCTTACTTCCAGCA (SEQ ID NO: 2688) |
| | | | | | CAAACTGCTGTCTCTTAAATCTCTT (SEQ ID NO: 2689) |
| | | | | | AAACTCTCTTCTTCCATTAAAATGT (SEQ ID NO: 2690) |
| 205078_at | PIGF | MGC32646, | phosphatidylinositol | 5281 | TGTATCTACTTTCTTATGTCTTGTT (SEQ ID NO: 2691) |
| | | MGC33136 | glycan anchor | | TACTTTCTTATGTCTTGTTTCTCCT (SEQ ID NO: 2692) |
| | | | biosynthesis, class F | | ATTTTTGTTCTGTATGGAGCACCAC (SEQ ID NO: 2693) |
| | | | | | GGAGCACCACTGATAGAGTTGGCAT (SEQ ID NO: 2694) |
| | | | | | GTGCCTTGCTTATGTTTGTTAGGAC (SEQ ID NO: 2695) |
| | | | | | TTTGTTAGGACCAAACCTCAAAGCA (SEQ ID NO: 2696) |
| | | | | | GAGAATAGTCTCCAGATCACTACAA (SEQ ID NO: 2697) |
| | | | | | CAGATCACTACAATTTCTAGCTTTG (SEQ ID NO: 2698) |
| | | | | | AGGAGCATGGCTTGGAGCACTTCCT (SEQ ID NO: 2699) |
| | | | | | GCACTTCCTATTCCACTGGATTGGG (SEQ ID NO: 2700) |
| | | | | | AAGACCATGGCAGGTATGGCCCATC (SEQ ID NO: 2701) |
| 204213_at | PIGR | FLJ22667, | polymeric | 5284 | TTTGTTGCCACCACTGAGAGCACCA (SEQ ID NO: 2702) |
| | | MGC125361, | immunoglobulin | | CCCCCAGGAAGCCTAGACGGTGTCG (SEQ ID NO: 2703) |
| | | MGC125362 | receptor | | CACCCATGACAATCACCTTCAGAAT (SEQ ID NO: 2704) |
| | | | | | CAGAATCATGTCGATCCTGGGGGCC (SEQ ID NO: 2705) |
| | | | | | GCTCTAACACCTGCCTAGGTTTTTC (SEQ ID NO: 2706) |
| | | | | | TTCCTACTGTCCTCAGAGGCGTGCT (SEQ ID NO: 2707) |
| | | | | | TCAAAGCCTGGCCTAATTGTTCCTA (SEQ ID NO: 2708) |
| | | | | | CATGAGGAGGTCCCACTTGCAACTT (SEQ ID NO: 2709) |
| | | | | | GGTCCTCATGGGTCCCTTGAAGGAA (SEQ ID NO: 2710) |
| | | | | | AGAAAGGAGAGACGTGCAGCGCCCC (SEQ ID NO: 2711) |
| | | | | | CCCTCTGCACCCTTATCATGGGATG (SEQ ID NO: 2712) |
| 217770_at | PIGT | CGI-06, | phosphatidylinositol | 51604 | CAGCATGCCCTACAACGTGATCTGC (SEQ ID NO: 2713) |
| | | FLJ41596, | glycan anchor | | ACAACGTGATCTGCCTCACGTGCAC (SEQ ID NO: 2714) |
| | | MGC8909, NDAP | biosynthesis, class T | | ACTGTGGTGGCCGTGTGCTACGGCT (SEQ ID NO: 2715) |
| | | | | | TGGCCAAGCGGCTGGCCAACCTTAT (SEQ ID NO: 2716) |
| | | | | | CTCTCCTCAGAGTTGGCTTTTGAAC (SEQ ID NO: 2717) |
| | | | | | GGCTTTTGAACCAAAGTGCCCTGGA (SEQ ID NO: 2718) |
| | | | | | TCCAGTACAGGAGCCACGAGCCAAA (SEQ ID NO: 2719) |
| | | | | | CCTGTAGTGGCCACCTCTATATTGA (SEQ ID NO: 2720) |
| | | | | | AAAAGTGGTCGGTGGCTGCTGTATT (SEQ ID NO: 2721) |
| | | | | | AGAAAGGTCGGCTGGCAGCACTGGC (SEQ ID NO: 2722) |
| | | | | | GGTGATGGGGTGTGCTACACAGTGT (SEQ ID NO: 2723) |
| 217620_s_at | PIK3CB | DKFZp779K1237, | phosphoinositide-3- | 5291 | AAGCAGATGTTATGGAAGCAAGTTC (SEQ ID NO: 2724) |
| | | MGC133043, | kinase, catalytic, beta | | GGAAGCAAGTTCACAATTACCCAAT (SEQ ID NO: 2725) |
| | | PI3K, PI3KCB, | polypeptide | | ATTACCCAATGTTCAACCTCCTTAT (SEQ ID NO: 2726) |
| | | PI3Kbeta, | | | AATGTTCAACCTCCTTATGGATATT (SEQ ID NO: 2727) |
| | | PIK3C1, p110- | | | TGCATGTGTGAATCAGACTGCTGTA (SEQ ID NO: 2728) |
| | | BETA | | | CAGACTGCTGTATATGAGGAGCTTG (SEQ ID NO: 2729) |
| | | | | | GAAACACGAAGACTCTGTGATGTCA (SEQ ID NO: 2730) |
| | | | | | TGTGATGTCAGACCTTTTCTTCCAG (SEQ ID NO: 2731) |
| | | | | | TTCTTCCAGTTCTCAAATTAGTGAC (SEQ ID NO: 2732) |
| | | | | | GACAAGAAGTTGTGACCCAGGGGAA (SEQ ID NO: 2733) |
| | | | | | CTCAAAAATTGGAGTCCTTATAGGA (SEQ ID NO: 2734) |
| 207469_s_at | PIR | | pirin (iron-binding | 8544 | ACTCGCACACCAACCTTATATTTGG (SEQ ID NO: 2735) |
| | | | nuclear protein) | | GGACTTCAAATTGGACCCAGGAGCC (SEQ ID NO: 2736) |
| | | | | | GGACAAGCTTCATTTACACGATATC (SEQ ID NO: 2737) |
| | | | | | GGAGATGTGTATATTGGGCCCGATG (SEQ ID NO: 2738) |
| | | | | | ATTGGGCCCGATGATGCACAACAAA (SEQ ID NO: 2739) |
| | | | | | CTCATCACACAGCAGTGCTTGGAGA (SEQ ID NO: 2740) |
| | | | | | GAAGCCACTTTGTCTTAATTGCTGG (SEQ ID NO: 2741) |
| | | | | | GAGAACCAGTTATCCAACATGGTCC (SEQ ID NO: 2742) |
| | | | | | CGGAAGAGCAGGTCTTGATGTGTCC (SEQ ID NO: 2743) |
| | | | | | GAAGGCATTCCATTTCTAAAGCTTA (SEQ ID NO: 2744) |
| | | | | | GCTTCTAAAGAATTCCACACTAACG (SEQ ID NO: 2745) |
| 210183_x_at | PNN | DRS, SDK3, | pinin, desmosome | 5411 | GGCAAATCTTTAAGTCTGTTCCCTT (SEQ ID NO: 2746) |
| | | memA, pinin | associated protein | | TCTGTTCCCTTCTAATTCTGTATCA (SEQ ID NO: 2747) |
| | | | | | ACATTGCTTTTGGCGGCCGCGAATT (SEQ ID NO: 2748) |
| | | | | | GCTCGCCCGCCAGAAGAATATGAAA (SEQ ID NO: 2749) |
| | | | | | AAGCGCCGAGATGACGGGCTTTCTG (SEQ ID NO: 2750) |
| | | | | | CCGCCCGCAAGCAGAGGGACTCGGA (SEQ ID NO: 2751) |
| | | | | | GGGACTCGGAGATCATGCAGCAGAA (SEQ ID NO: 2752) |
| | | | | | GGAACCCAAGTAGCTTTGTGGCTTC (SEQ ID NO: 2753) |
| | | | | | TCCTCCTGTAGTGCTCACAGGTCCC (SEQ ID NO: 2754) |
| | | | | | CTTCCCAGTGTTTTTTATTCCTGTG (SEQ ID NO: 2755) |
| | | | | | TTATTCCTGTGGGGCTCACCCCAAA (SEQ ID NO: 2756) |
| 206789_s_at | POU2F1 | OCT1, OTF1 | POU class 2 | 5451 | GGTGGCTCTCTTCCAATAACATCAC (SEQ ID NO: 2757) |
| | | | homeobox 1 | | GCTCTCTTCCAATAACATCACTTGA (SEQ ID NO: 2758) |
| | | | | | CATCACTTGATGCAACTGGGAACCT (SEQ ID NO: 2759) |
| | | | | | GCAACTGGGAACCTGGTATTTGCCA (SEQ ID NO: 2760) |
| | | | | | TATTTGCCAATGCGGGAGGAGCCCC (SEQ ID NO: 2761) |
| | | | | | TGCAGGGAACTCTGCACCTGTAGCC (SEQ ID NO: 2762) |
| | | | | | GCCTCCAAGGCACAGTGAGCTGGGC (SEQ ID NO: 2763) |
| | | | | | GCCTTTTTCACTCTGCAGTGTGATT (SEQ ID NO: 2764) |
| | | | | | TGTGATTGGACTGCCAGCCAGGTTA (SEQ ID NO: 2765) |
| | | | | | TTCCTCTCGCCGTGTTGTGAGGGCA (SEQ ID NO: 2766) |
| | | | | | TGGAGACGGAACATTTGCCTAATTT (SEQ ID NO: 2767) |
| 218341_at | PPCS | RP11-163G10.1, | phosphopantothenoyl | 79717 | AAACCAAGTTATTGCTATCAAGGAA (SEQ ID NO: 2768) |
| | | FLJ11838, | cysteine synthetase | | TAATCTTCAGTCTCGACACACAGCT (SEQ ID NO: 2769) |
| | | MGC117357, | | | GTCTCGACACACAGCTTTTATAGGT (SEQ ID NO: 2770) |
| | | MGC138220 | | | AAATTGTTCAGGGCTCTTAGAGATG (SEQ ID NO: 2771) |
| | | | | | AAACCATGGCTTTCATATGGACAGA (SEQ ID NO: 2772) |
| | | | | | AAGGCAGTGGTGTGTAGGCAAATAT (SEQ ID NO: 2773) |
| | | | | | AAATATGGTTTGGCATTCGTCTTTT (SEQ ID NO: 2774) |
| | | | | | GGCATTCGTCTTTTAATGACACCTG (SEQ ID NO: 2775) |
| | | | | | TACATGGGGGCCTGAATGTCAGCCA (SEQ ID NO: 2776) |
| | | | | | GAATGGTCATGCTTTGGAGATCAAA (SEQ ID NO: 2777) |
| | | | | | TGAATGCCTATGTATGTCAGGCCCT (SEQ ID NO: 2778) |
| 204228_at | PPIH | CYP-20, CYPH, | peptidylprolyl | 10465 | ATGGAGATGGTACTGGAGTCGCCAG (SEQ ID NO: 2779) |
| | | MGC5016, | isomerase H | | TCGCCAGTATTTACCGGGGGCCATT (SEQ ID NO: 2780) |
| | | SnuCyp-20, | (cyclophilin H) | | TTTAAACTTAGACACTCAGCTCCAG (SEQ ID NO: 2781) |
| | | USA-CYP | | | GCTTTCCATGGCGAACAGTGGTCCA (SEQ ID NO: 2782) |
| | | | | | ATCACCTGCTCTAAGTGCGATTGGC (SEQ ID NO: 2783) |
| | | | | | AAAATCATCGATGGACTTCTAGTGA (SEQ ID NO: 2784) |
| | | | | | TTCCCACAGGCCCCAACAATAAGCC (SEQ ID NO: 2785) |
| | | | | | CTACCTGTGGTGATCTCGCAGTGTG (SEQ ID NO: 2786) |
| | | | | | AAAGACTGAATCAGGCCTTCCCTTC (SEQ ID NO: 2787) |
| | | | | | AGTAAGATAATCTGGACTGGCCCCC (SEQ ID NO: 2788) |
| | | | | | CTGCTGCCCCATTTGATCAAGAGAC (SEQ ID NO: 2789) |
| 218273_s_at | PPM2C | RP23-203A12.1, | protein phosphatase | 54704 | ATTGACTTTGCAGCAGGGTGGCAGG (SEQ ID NO: 2790) |
| | | Gm1024 | 2C, magnesium- | | GTCCTGCCTAGCTCAGATTTCATGG (SEQ ID NO: 2791) |
| | | | dependent, catalytic | | CAGATTTCATGGCACCTGCACTTGA (SEQ ID NO: 2792) |
| | | | subunit | | CACTTCTTTATCACAGGTGTCTTGA (SEQ ID NO: 2793) |
| | | | | | TAGCTTCTTTTACCAACCTGAGAAA (SEQ ID NO: 2794) |
| | | | | | GTATCTTGCTGTGTGTAGTCTCTTG (SEQ ID NO: 2795) |
| | | | | | TCACACCTTTCTTCACTGAGATTCC (SEQ ID NO: 2796) |
| | | | | | TCCAGTTCTTCAAGCCATAAATGAC (SEQ ID NO: 2797) |
| | | | | | GATTATTTGACTGGAATGCTTCTTA (SEQ ID NO: 2798) |
| | | | | | GAATAACTATACTCCGTTATCCACC (SEQ ID NO: 2799) |
| | | | | | TATCCACCCGATTTCCTAATGTAAT (SEQ ID NO: 2800) |
| 202313_at | PPP2R2A | B55-ALPHA, | protein phosphatase | 5520 | GTTTGACAGTGTGCCATTCGACAAC (SEQ ID NO: 2801) |
| | | B55A, FLJ26613, | 2 (formerly 2A), | | GATTCATCACTGTGGTGTTCTCCAT (SEQ ID NO: 2802) |
| | | MGC52248, | regulatory subunit B, | | CTCCATGTCTGCTAGCCATTTAGGT (SEQ ID NO: 2803) |
| | | PR52A, PR55A | alpha isoform | | CTTCTTGCACCATCTTGCCTAATGG (SEQ ID NO: 2804) |
| | | | | | AACATTGATTTACTCCACTTTTTAT (SEQ ID NO: 2805) |
| | | | | | TATGCCTTCCATTGTGATGACGTCA (SEQ ID NO: 2806) |
| | | | | | AAGCCTTCAGTCATGCTATGGGATT (SEQ ID NO: 2807) |
| | | | | | GTGTATCCTCATTACTGTATCATTT (SEQ ID NO: 2808) |
| | | | | | TGTATCATTTGTGGGGTACACCCCT (SEQ ID NO: 2809) |
| | | | | | TTACTGTGGCTTGTAGCATTCCTCC (SEQ ID NO: 2810) |
| | | | | | CTCCACCCGGTCTTGGTGGTGGTAT (SEQ ID NO: 2811) |
| 209817_at | PPP3CB | RP11-345K20.1, | protein phosphatase | 5532 | TAGAGTTAACTACCTGCTTAGGCCC (SEQ ID NO: 2812) |
| | | CALNA2, CALNB | 3 (formerly 2B), | | GCTTAGGCCCCAGAACATTACTTAT (SEQ ID NO: 2813) |
| | | | catalytic subunit, beta | | AGAACATTACTTATGCCCTTCAGTT (SEQ ID NO: 2814) |
| | | | isoform | | TGTGCAAGGTTTTGTACCCTGGTAA (SEQ ID NO: 2815) |
| | | | | | AGTTTGTTTTCTGTGGTGTTTGTCA (SEQ ID NO: 2816) |
| | | | | | AATTAACTGTCTGTAACATGCTGTT (SEQ ID NO: 2817) |
| | | | | | CTGTCTGTCTTTCTTTAGGTTAGCT (SEQ ID NO: 2818) |
| | | | | | ACACTTGTCTGTCTTGCAATGTAGA (SEQ ID NO: 2819) |
| | | | | | GAAGCAGCTTTGTAGCACCTTGTTT (SEQ ID NO: 2820) |
| | | | | | TGCTGCATTTGTTGCTGCACTTTGT (SEQ ID NO: 2821) |
| | | | | | GCACTTTGTGCATTCTGAACATGAA (SEQ ID NO: 2822) |
| 205277_at | PRDM2 | RP5-1177E19.1, | PR domain | 7799 | AATGGTGGTCATTGGCCGACATCAC (SEQ ID NO: 2823) |
| | | HUMHOXY1, | containing 2, with | | CCGACATCACAGTTTTCCTGTTTCC (SEQ ID NO: 2824) |
| | | KMT8, MTB-ZF, | ZNF domain | | CTACAGCTAACCCTCACAAGCATGA (SEQ ID NO: 2825) |
| | | RIZ, RIZ1, RIZ2 | | | AGCATGAAGTGCTGTGGCTGTTCCT (SEQ ID NO: 2826) |
| | | | | | GGCTGTTCCTTATCCTAATGATGCG (SEQ ID NO: 2827) |
| | | | | | CTAATGATGCGCTTTTGTCCCGTAA (SEQ ID NO: 2828) |
| | | | | | GCAAGGAAAGCTCACGAACCCCAAA (SEQ ID NO: 2829) |
| | | | | | CTGCAGCCCATGGTGAGCTCTGGGA (SEQ ID NO: 2830) |
| | | | | | GGTCACTCCTTTTTTGCATGTGCAA (SEQ ID NO: 2831) |
| | | | | | AAAGTGGCATTTCCTGGTCAGTGGT (SEQ ID NO: 2832) |
| | | | | | AAGACGACAGCTCTGTATCTGCCAT (SEQ ID NO: 2833) |
| 37022_at | PRELP | MGC45323, | proline/arginine-rich | 5549 | ACCGGATCTGCTCTGACCGCACTTG (SEQ ID NO: 2834) |
| | | MST161, | end leucine-rich | | GCTCTGACCGCACTTGAAGGCTGGG (SEQ ID NO: 2835) |
| | | MSTP161, | repeat protein | | TGTGCCGGCCATTCGTTTTCTCTCT (SEQ ID NO: 2836) |
| | | SLRR2A | | | TGCCGGCCATTCGTTTTCTCTCTCT (SEQ ID NO: 2837) |
| | | | | | CACCCTCGAGGCAGGGAAAAGCCAT (SEQ ID NO: 2838) |
| | | | | | CCAGTTGAAAGACACCCAGTGCACA (SEQ ID NO: 2839) |
| | | | | | ACATCTGGGCCGTGGACTATCTGAT (SEQ ID NO: 2840) |
| | | | | | CGTGGACTATCTGATCTTGGTCCTG (SEQ ID NO: 2841) |
| | | | | | CTATCTGATCTTGGTCCTGGCCGAG (SEQ ID NO: 2842) |
| | | | | | CAGCCCGGAGTAAGGTTCAGAAAGT (SEQ ID NO: 2843) |
| | | | | | CGAGAAACCCAGTTCTCCTGGGCCA (SEQ ID NO: 2844) |
| | | | | | TCCTGGGCCAAGGATCCCTTCTGGC (SEQ ID NO: 2845) |
| | | | | | TTCTGGCAAAGCTGCTGCTCCGCCG (SEQ ID NO: 2846) |
| | | | | | GCAAAGCTGCTGCTCCGCCGCTTGG (SEQ ID NO: 2847) |
| | | | | | CAAAGCTGCTGCTCCGCCGCTTGGT (SEQ ID NO: 2848) |
| | | | | | GTAAGGGCTCCCAGGTCTTCATCTG (SEQ ID NO: 2849) |
| 215194_at | PRKCA | AAG6, | protein kinase C, | 5578 | GGAGGGTACCAGATGACAGATTCAC (SEQ ID NO: 2850) |
| | | MGC129900, | alpha | | ACAGATTCACCCAACACTCATCTGA (SEQ ID NO: 2851) |
| | | MGC129901, | | | AACTGCTGGACCATGGTTGGGTTAG (SEQ ID NO: 2852) |
| | | PKC-alpha, | | | TGATTAGGTTTATCCCGGCCTTTCA (SEQ ID NO: 2853) |
| | | PKCA, PRKACA | | | CCCGGCCTTTCAGATTCTAGGGGCT (SEQ ID NO: 2854) |
| | | | | | GATTCTAGGGGCTGTTTCCATGGAG (SEQ ID NO: 2855) |
| | | | | | ATGGGGCAACAGTCTGCTTGCTTTC (SEQ ID NO: 2856) |
| | | | | | GAAGGGTTAACCTAGAGCCAGACGT (SEQ ID NO: 2857) |
| | | | | | GAGCCAGACGTAACTTCTACAGAAT (SEQ ID NO: 2858) |
| | | | | | AACTGGACACTGAGCATGACGTACC (SEQ ID NO: 2859) |
| | | | | | ATGACGTACCATCTTCCACTAAGAT (SEQ ID NO: 2860) |
| 209158_s_at | PSCD2 | ARNO, CTS18, | pleckstrin homology, | 9266 | GCCTCTACTACTTTGAGTACACCAC (SEQ ID NO: 2861) |
| | | CTS18.1, | Sec7 and coiled-coil | | GGACAAGGAGCCCCGAGGAATCATC (SEQ ID NO: 2862) |
| | | CYTH2, | domains 2 | | GAATCATCCCCCTGGAGAATCTGAG (SEQ ID NO: 2863) |
| | | PSCD2L, SEC7L, | (cytohesin-2) | | GAATCTGAGCATCCGAGAGGTGGAC (SEQ ID NO: 2864) |
| | | Sec7p-L, Sec7p- | | | CTGCTTTGAACTTTACATCCCCAAC (SEQ ID NO: 2865) |
| | | like | | | ACAAGGGGCAGCTCATCAAAGCCTG (SEQ ID NO: 2866) |
| | | | | | GAGGGAAACCACATGGTGTACCGGA (SEQ ID NO: 2867) |
| | | | | | GAGTGGATCAAGTCCATCCAGGCGG (SEQ ID NO: 2868) |
| | | | | | TCCATCCAGGCGGCTGTGAGTGTGG (SEQ ID NO: 2869) |
| | | | | | CCATTATTTATTACGGAGCTGCCCC (SEQ ID NO: 2870) |
| | | | | | AACACGCTGTTGGTAATCTTATTAA (SEQ ID NO: 2871) |
| 203396_at | PSMA4 | HC9, HsT17706, | proteasome | 5685 | GGCTTTCAGCTCTATCAGAGTGACC (SEQ ID NO: 2872) |
| | | MGC111191, | (prosome, | | AGAGTGACCCTAGTGGAAATTACGG (SEQ ID NO: 2873) |
| | | MGC12467, | macropain) subunit, | | GGGGGATGGAAGGCCACATGCATTG (SEQ ID NO: 2874) |
| | | MGC24813, | alpha type, 4 | | GAAATAATAGCGCTGCAGCTGTGTC (SEQ ID NO: 2875) |
| | | PSC9 | | | GCGCTGCAGCTGTGTCAATGTTGAA (SEQ ID NO: 2876) |
| | | | | | GAAATGACCTTGAAGTCAGCACTTG (SEQ ID NO: 2877) |
| | | | | | CATGGATGTTAGTAAACTCTCTGCT (SEQ ID NO: 2878) |
| | | | | | AAGACAGTAATCAGAGTTCTCAAAC (SEQ ID NO: 2879) |
| | | | | | TACTGAATTGGGTCCTTGTCATTTC (SEQ ID NO: 2880) |
| | | | | | GTCCTTGTCATTTCTGTCCAATTGA (SEQ ID NO: 2881) |
| | | | | | TACCCTTCATGGACGTCTTAATCTT (SEQ ID NO: 2882) |
| 201274_at | PSMA5 | MGC117302, | proteasome | 5686 | CAAGCTGTGTCCAATCTGGCTTTGC (SEQ ID NO: 2883) |
| | | MGC125802, | (prosome, | | ATGTCTCGTCCCTTTGGAGTAGCAT (SEQ ID NO: 2884) |
| | | MGC125803, | macropain) subunit, | | AAAGGACCCCAGCTGTTTCATATGG (SEQ ID NO: 2885) |
| | | MGC125804, | alpha type, 5 | | TGGACCCATCTGGGACCTTTGTACA (SEQ ID NO: 2886) |
| | | PSC5, ZETA | | | GCTCGAGCAATTGGCTCTGCTTCAG (SEQ ID NO: 2887) |
| | | | | | CTGCTTCAGAGGGTGCCCAGAGCTC (SEQ ID NO: 2888) |
| | | | | | GAGCTCCTTGCAAGAACTTTACCAC (SEQ ID NO: 2889) |
| | | | | | GAAGCCATCAAGTCTTCACTCATCA (SEQ ID NO: 2890) |
| | | | | | ATTGAGCTAGCCACAGTGCAGCCTG (SEQ ID NO: 2891) |
| | | | | | AGGAATCCTGATCCTCAGAACTTCT (SEQ ID NO: 2892) |
| | | | | | TAAATTTTATTTCCAGCTCCTGTTC (SEQ ID NO: 2893) |
| 201114_x_at | PSMA7 | RP5-1005F21.4, | proteasome | 5688 | CCAGAGCCACCGGCTGACTGTAGAG (SEQ ID NO: 2894) |
| | | C6, HSPC, | (prosome, | | GGACCCGGTCACTGTGGAGTACATC (SEQ ID NO: 2895) |
| | | MGC3755, RC6- | macropain) subunit, | | CGCTACATCGCCAGTCTGAAGCAGC (SEQ ID NO: 2896) |
| | | 1, XAPC7 | alpha type, 7 | | CAGAGCAATGGGCGCAGGCCGTTTG (SEQ ID NO: 2897) |
| | | | | | CCCTCATCGTGGGTTTCGACTTTGA (SEQ ID NO: 2898) |
| | | | | | TTCGACTTTGATGGCACTCCTAGGC (SEQ ID NO: 2899) |
| | | | | | GGGCACATACCATGCCTGGAAGGCC (SEQ ID NO: 2900) |
| | | | | | ATAGGCCGGGGTGCCAAGTCAGTGC (SEQ ID NO: 2901) |
| | | | | | AAGCTGGTGATCAAGGCACTCCTGG (SEQ ID NO: 2902) |
| | | | | | GAACTTGCTGTCATGAGGCGAGATC (SEQ ID NO: 2903) |
| | | | | | ATGAGTCTCGATGTGTAGGCCTTTC (SEQ ID NO: 2904) |
| 200039_s_at | PSMB2 | RP23-120H2.2, | proteasome | 5690 | TAACTTCACACGCCGAAACCTGGCT (SEQ ID NO: 2905) |
| | | AU045357, | (prosome, | | CGAAACCTGGCTGACTGTCTTCGGA (SEQ ID NO: 2906) |
| | | AW108089, C7-I, | macropain) subunit, | | GGGCCAGCGCTGTATTACATGGACT (SEQ ID NO: 2907) |
| | | D4Wsu33e | beta type, 2 | | ACTCTCAGTATCCTCGACCGATACT (SEQ ID NO: 2908) |
| | | | | | TACACACCGACTATCTCACGTGAGA (SEQ ID NO: 2909) |
| | | | | | GTGAGAGGGCAGTGGAACTCCTTAG (SEQ ID NO: 2910) |
| | | | | | GCTCCAGAAACGCTTCATCCTGAAT (SEQ ID NO: 2911) |
| | | | | | TGAATCTGCCAACCTTCAGTGTTCG (SEQ ID NO: 2912) |
| | | | | | TGACCTGGATAACATTTCCTTCCCC (SEQ ID NO: 2913) |
| | | | | | AACAGGGCTCCTAACATCATGTCCT (SEQ ID NO: 2914) |
| | | | | | TCTTTTCAGGCGCACTCTTGATAAA (SEQ ID NO: 2915) |
| 201388_at | PSMD3 | P58, RPN3, S3 | proteasome | 5709 | GATATCCACAACATGTCTGTCAAGG (SEQ ID NO: 2916) |
| | | | (prosome, | | TCTGTCAAGGCCATGAGGTTTCCTC (SEQ ID NO: 2917) |
| | | | macropain) 26S | | GTTTCCTCCCAAATCGTACAACAAG (SEQ ID NO: 2918) |
| | | | subunit, non-ATPase, 3 | | GGAACGGCGTGAGCGAGAACAGCAG (SEQ ID NO: 2919) |
| | | | | | GAACAGCAGGACTTGGAGTTTGCCA (SEQ ID NO: 2920) |
| | | | | | TTTCCCACACACAGCTCATATGCTG (SEQ ID NO: 2921) |
| | | | | | ACAGCTCATATGCTGCATTCGTGCA (SEQ ID NO: 2922) |
| | | | | | TGCTGCATTCGTGCAGGGGGTGGGG (SEQ ID NO: 2923) |
| | | | | | CCCAGCCGGTGACTTACTGTACAGC (SEQ ID NO: 2924) |
| | | | | | GATAGTTCTGTGTACTCCTTTAGGG (SEQ ID NO: 2925) |
| | | | | | TTAGGGAGTGGGGGACTAGAACTGG (SEQ ID NO: 2926) |
| 207805_s_at | PSMD9 | MGC8644, Rpn4, | proteasome | 5715 | TGTGGAGTTCGGCTCTGTGAACACC (SEQ ID NO: 2927) |
| | | p27 | (prosome, | | CCCAGAACTTCCAGTCACTGCATAA (SEQ ID NO: 2928) |
| | | | macropain) 26S | | GAATGTGACAGTGATCCGCAGGGGG (SEQ ID NO: 2929) |
| | | | subunit, non-ATPase, 9 | | CCAGCTTAGACTTGTTCCAACACGC (SEQ ID NO: 2930) |
| | | | | | AAAAGGACTGCTGGGCTGCAACATT (SEQ ID NO: 2931) |
| | | | | | GGGTCTAGGGATTTCCAACTTGTCT (SEQ ID NO: 2932) |
| | | | | | AGAGGCTTGTAACCTGAACTTCTGT (SEQ ID NO: 2933) |
| | | | | | GTGTGGTGGCAGTACTGTGGCCCAC (SEQ ID NO: 2934) |
| | | | | | CCCACCAGTGTAATCTCCCTGGATT (SEQ ID NO: 2935) |
| | | | | | TCTTTCACAAATTAGGCCACGGCCC (SEQ ID NO: 2936) |
| | | | | | GTGGGCGGAAGTTATTCTGGCAGGT (SEQ ID NO: 2937) |
| 218371_s_at | PSPC1 | RP11-523H24.2, | paraspeckle | 55269 | ATGTGGCTGAAGTTACCCGAAGTTC (SEQ ID NO: 2938) |
| | | DKFZp566B1447, | component 1 | | GTTACCCGAAGTTCAGCTTGCAGTG (SEQ ID NO: 2939) |
| | | FLJ10955, | | | AGTTCAGCTTGCAGTGTAATTCAGA (SEQ ID NO: 2940) |
| | | PSP1 | | | GAATCATACTTCCTTTCAGGTCAAA (SEQ ID NO: 2941) |
| | | | | | GTGGGAAATATCCTGTTCGTGACCT (SEQ ID NO: 2942) |
| | | | | | TCCTGTTCGTGACCTAGGAGAAACA (SEQ ID NO: 2943) |
| | | | | | TTCTTACTCCATACAAGCAGTTTAT (SEQ ID NO: 2944) |
| | | | | | AAACATGCTACAGGGGCACATTTTC (SEQ ID NO: 2945) |
| | | | | | ACAGGGGCACATTTTCTGTGGTTAA (SEQ ID NO: 2946) |
| | | | | | GCAGTTTTCCTTTGCATTGAGGGTT (SEQ ID NO: 2947) |
| | | | | | CTTTGCATTGAGGGTTTTGGCTTTT (SEQ ID NO: 2948) |
| 210832_x_at | PTGER3 | EP3, EP3-I, EP3- | prostaglandin E | 5733 | GTCTCCGCTCCTGATAATGATGTTG (SEQ ID NO: 2949) |
| | | II, EP3-III, EP3- | receptor 3 (subtype | | ACTTCTTCTTAATAGCTGTTCGCCT (SEQ ID NO: 2950) |
| | | IV, EP3e, | EP3) | | TTCGCCTGGCTTCACTGAACCAGAT (SEQ ID NO: 2951) |
| | | MGC141828, | | | TCTTGGATCCTTGGGTTTACCTGCT (SEQ ID NO: 2952) |
| | | MGC141829, | | | GTTTTGCCAGATCAGGTACCACACA (SEQ ID NO: 2953) |
| | | MGC27302 | | | ACAAACAACTATGCATCCAGCTCCA (SEQ ID NO: 2954) |
| | | | | | CCAGTGTTCCTCAACCTTGATGTGG (SEQ ID NO: 2955) |
| | | | | | TAAAACCTGCCTTTCTGCCAGGATC (SEQ ID NO: 2956) |
| | | | | | GGATCACATCACTGGAAGCTCCATG (SEQ ID NO: 2957) |
| | | | | | GCTCCATGACTCTCTTTTTGTAAAA (SEQ ID NO: 2958) |
| | | | | | TCCCAAACTATTCTCTTTTACTTCT (SEQ ID NO: 2959) |
| 211756_at | PTHLH | HHM, | parathyroid hormone- | 5744 | AAAACTTCACATTTATGTGGCTTGT (SEQ ID NO: 2960) |
| | | MGC14611, PLP, | like hormone | | TGTGGCTTGTTTATCCTTAGCTCAC (SEQ ID NO: 2961) |
| | | PTHR, PTHRP | | | TGTTTATCCTTAGCTCACAGATTGA (SEQ ID NO: 2962) |
| | | | | | GGTAATAATGACACTCCTAGACTTT (SEQ ID NO: 2963) |
| | | | | | GACACTCCTAGACTTTGGGATCAAA (SEQ ID NO: 2964) |
| | | | | | TAACTTAGGGCCAAGTCTTGGGTCT (SEQ ID NO: 2965) |
| | | | | | GTTCACAACCTAGGGCAAGTTACTC (SEQ ID NO: 2966) |
| | | | | | AAGTTACTCTGCCTTTCTAAGACTC (SEQ ID NO: 2967) |
| | | | | | CTAAGACTCACTTACATCTTCTGTG (SEQ ID NO: 2968) |
| | | | | | TAATTGTACCAACCTCATAGAGTTT (SEQ ID NO: 2969) |
| | | | | | ATGTGGACTAAATATCTGTCATATA (SEQ ID NO: 2970) |
| 202990_at | PYGL | GSD6 | phosphorylase, | 5836 | AGAGCAGATTTCCACTGCAGGCACC (SEQ ID NO: 2971) |
| | | | glycogen; liver (Hers | | GTTCATGCTAAATGGGGCCCTAACT (SEQ ID NO: 2972) |
| | | | disease, glycogen | | GGCCCTAACTATCGGGACCATGGAT (SEQ ID NO: 2973) |
| | | | storage disease type | | AGAGAACCTGTTCATCTTTGGCATG (SEQ ID NO: 2974) |
| | | | VI) | | GGATAGATGATGTGGCTGCTTTGGA (SEQ ID NO: 2975) |
| | | | | | GCAGCCTGACCTCTTCAAAGATATC (SEQ ID NO: 2976) |
| | | | | | GTCTTTGCAGACTACGAAGCCTATG (SEQ ID NO: 2977) |
| | | | | | AAGGCCTGGAACACAATGGTACTCA (SEQ ID NO: 2978) |
| | | | | | GGTACTCAAAAACATAGCTGCCTCG (SEQ ID NO: 2979) |
| | | | | | ATAGCTGCCTCGGGGAAATTCTCCA (SEQ ID NO: 2980) |
| | | | | | GGAACGTGGAACCTTCAGATCTAAA (SEQ ID NO: 2981) |
| 212866_at | R3HCC1 | DKFZp564N123 | R3H domain and | 203069 | GACCTTGCCCACGTGGTAGAGATCT (SEQ ID NO: 2982) |
| | | | coiled-coil containing 1 | | GATCTATGACTTTGAACCAGCGCTC (SEQ ID NO: 2983) |
| | | | | | CCAGCGCTCAAGACGGAGGACCTGC (SEQ ID NO: 2984) |
| | | | | | GCTGGCAACGTTTTCTGAGTTCCAA (SEQ ID NO: 2985) |
| | | | | | GGGGTTCAGGATTCAGTGGGTGGAT (SEQ ID NO: 2986) |
| | | | | | GTGGGTGGATGATACTCACGCACTC (SEQ ID NO: 2987) |
| | | | | | GCAGTCAAAGCTCAAAGCCTTGCAG (SEQ ID NO: 2988) |
| | | | | | TGCAGAGGCCAAAACTCCTGCGTCT (SEQ ID NO: 2989) |
| | | | | | AAAGAAAGAGCGGCCTGCTGTCCGG (SEQ ID NO: 2990) |
| | | | | | CAACACCATAAGCCTTCACAGACGC (SEQ ID NO: 2991) |
| | | | | | CACCCTCGAGCTTCACCATGGGGTG (SEQ ID NO: 2992) |
| 221792_at | RAB6B | | RAB6B, member | 51560 | ACACTAGGTTCTTCCCAGCATATGA (SEQ ID NO: 2993) |
| | | | RAS oncogene family | | AGTCCCCAAGTCTACTCTGATTGAT (SEQ ID NO: 2994) |
| | | | | | TGATTGATTGTCATGGGCACCCACA (SEQ ID NO: 2995) |
| | | | | | AATGTGCAAACTCCTTGGACTCCAG (SEQ ID NO: 2996) |
| | | | | | TCCAGACCCCTGTCTGGTTGGCACT (SEQ ID NO: 2997) |
| | | | | | GCACTCCCAGCAGTGTGTGGTTTCA (SEQ ID NO: 2998) |
| | | | | | AGGTGGGAAGCCTCCATAGCATTGA (SEQ ID NO: 2999) |
| | | | | | AGCATTGAAGGCCAGCCTGCATCTC (SEQ ID NO: 3000) |
| | | | | | CACCCATCCCTTTAAAGTGATTGGC (SEQ ID NO: 3001) |
| | | | | | GAGGGTGGCAATTCCATCTGTTATG (SEQ ID NO: 3002) |
| | | | | | GACATAGCCGCCATGTTGATTTCAC (SEQ ID NO: 3003) |
| 204478_s_at | RABIF | MSS4, | RAB interacting | 5877 | TGTGGCCTTGGAACGAGTTTCCCAT (SEQ ID NO: 3004) |
| | | RASGFR3, | factor | | TCTCCAAAGATAAACCTACTCCCCA (SEQ ID NO: 3005) |
| | | RASGRF3 | | | TCCCCACAAGAACTGGCCTTTAATG (SEQ ID NO: 3006) |
| | | | | | AATGTGGTATAACTGTTCCGCTGCC (SEQ ID NO: 3007) |
| | | | | | ACTGAGTACCAGCATGTCCATTTGA (SEQ ID NO: 3008) |
| | | | | | CCTCCTGCTTAGTTCACTTTGTATC (SEQ ID NO: 3009) |
| | | | | | TCCCCCAGTTTTGGGACACTGTGCT (SEQ ID NO: 3010) |
| | | | | | ACACTGTGCTTACCTCCAAAAATCT (SEQ ID NO: 3011) |
| | | | | | TCCAAAAATCTCATCTCTTCCCTGG (SEQ ID NO: 3012) |
| | | | | | GAGTGACTTTGTTGTATGGCTACCC (SEQ ID NO: 3013) |
| | | | | | TATGGCTACCCTTGTGATCTACAGT (SEQ ID NO: 3014) |
| 205326_at | RAMP3 | | receptor (G protein- | 10268 | TCTGCCTGCAGGTTTCTATGCTGTT (SEQ ID NO: 3015) |
| | | | coupled) activity | | TCTTAGCACAGAATCCAGCCTAGCC (SEQ ID NO: 3016) |
| | | | modifying protein 3 | | CCTAGCCTTAGCCGCAGTCTAGGCC (SEQ ID NO: 3017) |
| | | | | | TCTAGGCCCTGCTTGGACTAGGACT (SEQ ID NO: 3018) |
| | | | | | TTGGACTAGGACTCCTTGCTTGACC (SEQ ID NO: 3019) |
| | | | | | GGCTCTTGGAAGACGTTCCGTGCTG (SEQ ID NO: 3020) |
| | | | | | GCAGAGTCCATTCCATCATGATGCT (SEQ ID NO: 3021) |
| | | | | | CATGATGCTGTGCCCGCTATGGGCT (SEQ ID NO: 3022) |
| | | | | | TGCCCGCTATGGGCTGTGTCCATGA (SEQ ID NO: 3023) |
| | | | | | GTGGGGGTGTGTTATAGCCCCTCAC (SEQ ID NO: 3024) |
| | | | | | CCCTCTGGAATGGCATCCCATGAGC (SEQ ID NO: 3025) |
| 202033_s_at | RB1CC1 | CC1, | RB1-inducible coiled- | 9821 | TGATTTGTCCTGCAGTGCTCATTCA (SEQ ID NO: 3026) |
| | | DRAGOU14, | coil 1 | | AACAGCAGGCCATCTTTTTATGCAA (SEQ ID NO: 3027) |
| | | FIP200 | | | ACTTCACTGGTGTACATCGTTTACT (SEQ ID NO: 3028) |
| | | | | | TCGTTTACTTTTTAACTGGCTTCAT (SEQ ID NO: 3029) |
| | | | | | AGAAATGCGGACCAAACTACTTCAT (SEQ ID NO: 3030) |
| | | | | | ACTACTTCATTTTCTCAAAGGGCAT (SEQ ID NO: 3031) |
| | | | | | GCATACCTTGTGCATTGTGGCTTAT (SEQ ID NO: 3032) |
| | | | | | GATGAGCCATATTAATTGCCTGTTA (SEQ ID NO: 3033) |
| | | | | | GTTATTATTACCAGCATTTGTCCTT (SEQ ID NO: 3034) |
| | | | | | ATACTTGCACTCTTTAACACATTCT (SEQ ID NO: 3035) |
| | | | | | AGAGGAGTGTTATTGCATGCTGATA (SEQ ID NO: 3036) |
| 216215_s_at | RBM9 | FOX2, Fox-2, | RNA binding motif | 23543 | ACATGCAACATGCTGTTCCTCCAGC (SEQ ID NO: 3037) |
| | | HNRBP2, | protein 9 | | GTACCTTTGGGGCATGGGGGCATTA (SEQ ID NO: 3038) |
| | | HRNBP2, RTA, | | | GGGATGCTTGTGTAATCGACCACCT (SEQ ID NO: 3039) |
| | | dJ106I20.3, fxh | | | TCCAGAGAAGCATCCCAGGGCTTTG (SEQ ID NO: 3040) |
| | | | | | GGCTTTGTGACAGTCTCTAATTCCC (SEQ ID NO: 3041) |
| | | | | | TCCCTTCCCTTCTCGTTAAGAATCA (SEQ ID NO: 3042) |
| | | | | | GTATTCATTGGGTTACTCCTATTAT (SEQ ID NO: 3043) |
| | | | | | AGATCTTTAGCTTTTATTCCTTATT (SEQ ID NO: 3044) |
| | | | | | TTATTGATCATGTTGCTGGCTTTTA (SEQ ID NO: 3045) |
| | | | | | GCGAAGGAGGAGCACTGCCTCAGCC (SEQ ID NO: 3046) |
| | | | | | GCCTCAGCCTTTGCACATGGTAATG (SEQ ID NO: 3047) |
| 210568_s_at | RECQL | RECQL1, RecQ1 | RecQ protein-like | 5965 | AACAGAGTACTGCAGAGATCTAATC (SEQ ID NO: 3048) |
| | | | (DNA helicase Q1- | | AAAACTCACTCCATTGAAACTGATT (SEQ ID NO: 3049) |
| | | | like) | | GAAACTGATTGATTCTTGGATGGGA (SEQ ID NO: 3050) |
| | | | | | GGATGGGAAAGGGTGCAGCAAAACT (SEQ ID NO: 3051) |
| | | | | | TCCCACACTTCCTCGTGAAGATCTG (SEQ ID NO: 3052) |
| | | | | | GGAGAAGATTATTGCACACTTTCTA (SEQ ID NO: 3053) |
| | | | | | AGAACTCTTTCAGGGCTGAATCGTC (SEQ ID NO: 3054) |
| | | | | | AGGGCTGAATCGTCTCAAACTTGTC (SEQ ID NO: 3055) |
| | | | | | TCGTCTCAAACTTGTCATTCTGAAC (SEQ ID NO: 3056) |
| | | | | | CCAGAAGAAGGCTGCAAACATGCTT (SEQ ID NO: 3057) |
| | | | | | GATGATGCCTGATATGAATGTTACT (SEQ ID NO: 3058) |
| 218767_at | REXO4 | RP11-244N20.5, | REX4, RNA | 57109 | CATCTTCGGTGACACCCTGGGGTGA (SEQ ID NO: 3059) |
| | | REX4, XPMC2, | exonuclease 4 | | TCTCGAGGAGCCACATTTTCCTCCT (SEQ ID NO: 3060) |
| | | XPMC2H | homolog (S. cerevisiae) | | GGACCCAGACCAGGTGTCTGCAGTC (SEQ ID NO: 3061) |
| | | | | | ACATCAGGGGTTTCCTGTGGCCGTT (SEQ ID NO: 3062) |
| | | | | | GTTGGCTGGTGGGTCACCCAGCAGA (SEQ ID NO: 3063) |
| | | | | | GACTGCGGTTATTCCTGGAGGTCGG (SEQ ID NO: 3064) |
| | | | | | GGAGGTCGGCAGACATGCCAACCTT (SEQ ID NO: 3065) |
| | | | | | ATGCCAACCTTGGGCTATTTGAGCT (SEQ ID NO: 3066) |
| | | | | | TGTGATGCTAGCCGGTGGCTTTCTG (SEQ ID NO: 3067) |
| | | | | | GTTTGAGGCTCCCCTGGGAACTAGA (SEQ ID NO: 3068) |
| | | | | | CAAGGCGTTATTGGGCCACCTGACA (SEQ ID NO: 3069) |
| 208021_s_at | RFC1 | A1, MGC51786, | replication factor C | 5981 | TTGACCACGTCTCTGCTGTGAATAG (SEQ ID NO: 3070) |
| | | MHCBFB, PO- | (activator 1) 1, | | CTCAGACATTCTTATCCTTCTGGAG (SEQ ID NO: 3071) |
| | | GA, RECC1, | 145 kDa | | TCTTATCCTTCTGGAGTCTCAACCC (SEQ ID NO: 3072) |
| | | RFC, RFC140 | | | GAGTCTCAACCCATCAGGTTCAGAC (SEQ ID NO: 3073) |
| | | | | | AACCCATCAGGTTCAGACCAGTAGG (SEQ ID NO: 3074) |
| | | | | | GACCAGTAGGAACCAGGCTGGGTCA (SEQ ID NO: 3075) |
| | | | | | CAGGCTGGGTCAGGCTCTTAATTTC (SEQ ID NO: 3076) |
| | | | | | AGGCTCTTAATTTCACTACGGTGGG (SEQ ID NO: 3077) |
| | | | | | AAATGCTCAGAGGTCTGTGCTACCT (SEQ ID NO: 3078) |
| | | | | | TAAGGATCGTATTCCCTGCTTTAAG (SEQ ID NO: 3079) |
| | | | | | TGGTAGTCTTGTTTGGGTGATGAGA (SEQ ID NO: 3080) |
| 204336_s_at | RGS19 | GAIP, RGSGAIP | regulator of G-protein | 10287 | CAGTGGGGAGTGCTGTGTCTCCTGG (SEQ ID NO: 3081) |
| | | | signaling 19 | | GCCAAGCAGGAACTCCAGGTGCAGG (SEQ ID NO: 3082) |
| | | | | | TGGGGGCTCTTGCGTGGTGAGAGTA (SEQ ID NO: 3083) |
| | | | | | TGCGTGGTGAGAGTAGGGGTCCCCC (SEQ ID NO: 3084) |
| | | | | | TTGGTGGGGAACAGAACCTCCGCAT (SEQ ID NO: 3085) |
| | | | | | ACCTCCGCATCGTGTAGTTTTGTGA (SEQ ID NO: 3086) |
| | | | | | TACTTGAGCTGTCTGTACCCCAGAA (SEQ ID NO: 3087) |
| | | | | | TGTACCCCAGAATCAAACACAGAAC (SEQ ID NO: 3088) |
| | | | | | CTCAGAATCCTGCACTCAAGGTGGC (SEQ ID NO: 3089) |
| | | | | | TAAACCTGGAAACATGTCCTTACTA (SEQ ID NO: 3090) |
| | | | | | ACATGTCCTTACTAGGTGTTTTATC (SEQ ID NO: 3091) |
| 214811_at | RIMBP2 | KIAA0318, | RIMS binding protein 2 | 23504 | TGGTAAAGCACCAGTACTCCAACCT (SEQ ID NO: 3092) |
| | | MGC15831, | | | CTCCAACCTTCCAGAAAGCCGATTA (SEQ ID NO: 3093) |
| | | RBP2, RIM-BP2 | | | AGCCGATTATCTTCATTGCTTTTAA (SEQ ID NO: 3094) |
| | | | | | CTGTGGCATATGGTTTTCTGTTACT (SEQ ID NO: 3095) |
| | | | | | TTTCGTTGTCAAAATGCCATACCCA (SEQ ID NO: 3096) |
| | | | | | TCCACATAATGCATAAGCCACACCA (SEQ ID NO: 3097) |
| | | | | | GCCACACCAAAACCAGACTCAATTT (SEQ ID NO: 3098) |
| | | | | | CCAAATGAGTCCATACCCATCTTCA (SEQ ID NO: 3099) |
| | | | | | TTTTGTAATTCTTGTCCACTTGTAA (SEQ ID NO: 3100) |
| | | | | | CATACTTTTCAGACTGCCTTTCTTT (SEQ ID NO: 3101) |
| | | | | | ATCCATGTGTGGTTAAGCTCTGTGT (SEQ ID NO: 3102) |
| 212479_s_at | RMND5A | CTLH, FLJ12753, | required for meiotic | 64795 | AATACACCAGCAGTTGTCATTCAAT (SEQ ID NO: 3103) |
| | | FLJ13910, | nuclear division 5 | | ATGCAGGTTTTTGTACTTAATTATA (SEQ ID NO: 3104) |
| | | FLJ21795, | homolog A (S. cerevisiae) | | GCAGAAACGGAAATTGACCTCCCCG (SEQ ID NO: 3105) |
| | | MGC78451, | | | CTCCCCGCCATGTGTTTAATATTCC (SEQ ID NO: 3106) |
| | | RMD5, p44CTLH | | | CCATGTGTTTAATATTCCTCCTGCT (SEQ ID NO: 3107) |
| | | | | | ATCGTAAGCCTTGAGAGTGTTTGTG (SEQ ID NO: 3108) |
| | | | | | GATAGATTCCAGATATGCTTTTTGA (SEQ ID NO: 3109) |
| | | | | | GCACACCTGCATTTTAGCTCTGCAT (SEQ ID NO: 3110) |
| | | | | | GCAGGCTGCGTGTTTAAGAATTTCA (SEQ ID NO: 3111) |
| | | | | | CTGGCTGGTGTGAGAAGTCTTCCGT (SEQ ID NO: 3112) |
| | | | | | GAAGTCTTCCGTTAGCATAGAGTGG (SEQ ID NO: 3113) |
| 212696_s_at | RNF4 | RES4-26, | ring finger protein 4 | 6047 | CTGAAGGAGTGTCCCTCCTCTATGT (SEQ ID NO: 3114) |
| | | SNURF | | | GAAGAGGGCAGAATCCGCAGCTCTC (SEQ ID NO: 3115) |
| | | | | | TGTGATGTGTAGCATGTCTGCCCTC (SEQ ID NO: 3116) |
| | | | | | CCCTCTGACTGGACATCATTGCCAT (SEQ ID NO: 3117) |
| | | | | | CATCATTGCCATTAACTTTCTTCTG (SEQ ID NO: 3118) |
| | | | | | TTTCTTCTGGGCATCACGGCAATGT (SEQ ID NO: 3119) |
| | | | | | GTCACGATGCCCAGACTTGGAGCAA (SEQ ID NO: 3120) |
| | | | | | AGGCAACCTTGGAGTCAGTCCACTC (SEQ ID NO: 3121) |
| | | | | | ATGTACATAGTTCAACTCTTTCTTT (SEQ ID NO: 3122) |
| | | | | | TATCCATGGATATCAGTCTGCTTTG (SEQ ID NO: 3123) |
| | | | | | GACTCCTCTGCTAGTGTTACAGATG (SEQ ID NO: 3124) |
| 222279_at | RP3- | FLJ35429 | hypothetical protein | 285830 | AAATCTTAATCTGGACCCTTTTCAT (SEQ ID NO: 3125) |
| | 377H14.5 | | FLJ35429 | | GGACCCTTTTCATAAGATGGTGCCA (SEQ ID NO: 3126) |
| | | | | | AAAACAAGATCCACATGTCACCCAC (SEQ ID NO: 3127) |
| | | | | | TCCGGAGGCAGCCCAAATATCCTTG (SEQ ID NO: 3128) |
| | | | | | GAGAAATAAGCTTGACCACCATGTT (SEQ ID NO: 3129) |
| | | | | | ACCACCATGTTATCATCAAGTTTCC (SEQ ID NO: 3130) |
| | | | | | CGCTGACCACTGAGTAGGCTGTGAA (SEQ ID NO: 3131) |
| | | | | | ACAACAGCCTACTTGTGTTCACTGT (SEQ ID NO: 3132) |
| | | | | | AAGTCAACACTCTGATTCAGTCTGA (SEQ ID NO: 3133) |
| | | | | | CTCCTGACTACGATGCTTTGGTTGT (SEQ ID NO: 3134) |
| | | | | | GATGCTTTGGTTGTTGCCACCAAAA (SEQ ID NO: 3135) |
| 220960_x_at | RPL22 | EAP, HBP15, | ribosomal protein L22 | 6146 | AATTCTGATGTCGTACCTAAGGCTT (SEQ ID NO: 3136) |
| | | HBP15/L22 | | | ACCTAAGGCTTGTCCATCTTTGTTG (SEQ ID NO: 3137) |
| | | | | | TTGTTGGAGGTGCCATGGCTCCTGT (SEQ ID NO: 3138) |
| | | | | | TGAAGTTCACTCTTGATTGCACCCA (SEQ ID NO: 3139) |
| | | | | | ATTGCACCCACCCTGTAGAAGATGG (SEQ ID NO: 3140) |
| | | | | | GGAGCAAGAGCAAGATCACCGTGAC (SEQ ID NO: 3141) |
| | | | | | ATCACCGTGACATCCGAGGTGCCTT (SEQ ID NO: 3142) |
| | | | | | GAATAATCTACGTGACTGGTTGCGC (SEQ ID NO: 3143) |
| | | | | | CTGGTTGCGCGTAGTTGCTAACAGC (SEQ ID NO: 3144) |
| | | | | | TACGAATTACGTTACTTCCAGATTA (SEQ ID NO: 3145) |
| | | | | | TTTATCCTTGTATCTCTGCAGTGTG (SEQ ID NO: 3146) |
| 200937_s_at | RPL5 | MGC117339, | ribosomal protein L5 | 6125 | GTGGAGGTGACTGGTGATGAATACA (SEQ ID NO: 3147) |
| | | MSTP030 | | | GTGACTGGTGATGAATACAATGTGG (SEQ ID NO: 3148) |
| | | | | | AAGCATTGATGGTCAGCCAGGTGCC (SEQ ID NO: 3149) |
| | | | | | ATTGATGGTCAGCCAGGTGCCTTCA (SEQ ID NO: 3150) |
| | | | | | TGCCTTCACCTGCTATTTGGATGCA (SEQ ID NO: 3151) |
| | | | | | GCTATTTGGATGCAGGCCTTGCCAG (SEQ ID NO: 3152) |
| | | | | | AGGCCTTGCCAGAACTACCACTGGC (SEQ ID NO: 3153) |
| | | | | | CAGAACTACCACTGGCAATAAAGTT (SEQ ID NO: 3154) |
| | | | | | GCAGAAGTACATCGGAAGCACATCA (SEQ ID NO: 3155) |
| | | | | | TGCAGATTACATGCGCTACTTAATG (SEQ ID NO: 3156) |
| | | | | | TACATGCGCTACTTAATGGAAGAAG (SEQ ID NO: 3157) |
| 214167_s_at | RPLP0 /// | RPLP0 /// | ribosomal protein, | 6175 | CCATTGCCCCATGTGAAGTCACTGT (SEQ ID NO: 3158) |
| | RPLP0-like | RPLP0-like, | large, P0 /// | 220717 | CCCCATGTGAAGTCACTGTGCCAGC (SEQ ID NO: 3159) |
| | | L10E, | ribosomal protein P0- | | CCAGCCCAGAACACTGGTCTCGGGC (SEQ ID NO: 3160) |
| | | MGC111226, | like | | AGCCCAGAACACTGGTCTCGGGCCC (SEQ ID NO: 3161) |
| | | MGC88175, P0, | | | GAACACTGGTCTCGGGCCCGAGAAG (SEQ ID NO: 3162) |
| | | PRLP0, RPP0 | | | TGGTCTCGGGCCCGAGAAGACCTCC (SEQ ID NO: 3163) |
| | | | | | TTTTTCCAGGCTTTAGGTATCACCA (SEQ ID NO: 3164) |
| | | | | | GGTATCACCACTAAAATCTCCAGGG (SEQ ID NO: 3165) |
| | | | | | CACCACTAAAATCTCCAGGGGCACC (SEQ ID NO: 3166) |
| | | | | | TAAAATCTCCAGGGGCACCATTGAA (SEQ ID NO: 3167) |
| | | | | | ATCTCCAGGGGCACCATTGAAATCC (SEQ ID NO: 3168) |
| 211487_x_at | RPS17 | MGC72007, | ribosomal protein | 6218 | GCCAAGGCAGGCGATCACATGAGGT (SEQ ID NO: 3169) |
| | | RPS17L1, | S17 | | CGCGTTCGCACCAAAACCGTGAAGA (SEQ ID NO: 3170) |
| | | RPS17L2 | | | TAGAAAAGTACTACACGCGCCTGGG (SEQ ID NO: 3171) |
| | | | | | TGCGAGGAGATCGCCATTATCCCCA (SEQ ID NO: 3172) |
| | | | | | TATCCCCAGCAAAAAGCTCCGCAAC (SEQ ID NO: 3173) |
| | | | | | GATAGCAGGTTATGTCACGCATCTG (SEQ ID NO: 3174) |
| | | | | | GTAAGAGGTATCTCCATCAAGCTGC (SEQ ID NO: 3175) |
| | | | | | TCCTGAGGTCTCAGCCTTGGATCAG (SEQ ID NO: 3176) |
| | | | | | GCTTTTGGACTTCGGCAGTCTGTCC (SEQ ID NO: 3177) |
| | | | | | GTCACTCAGCCTACAGTTGGGATGA (SEQ ID NO: 3178) |
| | | | | | GATGAATTTCAAAACGCCTCGGGGA (SEQ ID NO: 3179) |
| 218007_s_at | RPS27L | 40S ribosomal | ribosomal protein | 51065 | GCTAGCTGTGTGGGCTGGGAGGTCT (SEQ ID NO: 3180) |
| | | protein S27-like | S27-like | | GCTGGGAGGTCTGGTAGGGCTGAGC (SEQ ID NO: 3181) |
| | | | | | AGAGGATCAACATGCCTTTGGCTAG (SEQ ID NO: 3182) |
| | | | | | TAGAGATTTACTACATCCGTCCTTG (SEQ ID NO: 3183) |
| | | | | | GAAACGCCTAGTACAAAGTCCAAAT (SEQ ID NO: 3184) |
| | | | | | GGATGTAAAATGTCCAGGTTGCTAC (SEQ ID NO: 3185) |
| | | | | | GTTGCTACAAGATCACCACGGTTTT (SEQ ID NO: 3186) |
| | | | | | GCCATGCTCAGACAGTGGTTCTTTG (SEQ ID NO: 3187) |
| | | | | | CACTAATGATTCAAACAGCTTCCTG (SEQ ID NO: 3188) |
| | | | | | GTGTTGTCTCACAGAAAGCCTTATC (SEQ ID NO: 3189) |
| | | | | | GGTATACAGCAGTAATCTCCTATTT (SEQ ID NO: 3190) |
| 200099_s_at | RPS3A /// | FTE1, MFTL, | ribosomal protein | 6189 | TTGAAGTGAGTCTTGCTGATTTGCA (SEQ ID NO: 3191) |
| | LOC439992 | MGC23240 /// | S3A /// similar to v- | 439992 | GAGTCTTGCTGATTTGCAGAATGAT (SEQ ID NO: 3192) |
| | | | fos transformation | | TCAAGCTGATTACTGAAGATGTTCA (SEQ ID NO: 3193) |
| | | | effector protein | | GTAAAAACTGCCTGACTAACTTCCA (SEQ ID NO: 3194) |
| | | | | | AAACTGCCTGACTAACTTCCATGGC (SEQ ID NO: 3195) |
| | | | | | GGCAGACAATGATTGAAGCTCACGT (SEQ ID NO: 3196) |
| | | | | | TTGAAGCTCACGTTGATGTCAAGAC (SEQ ID NO: 3197) |
| | | | | | TACGGAAGACCTCTTATGCTCAGCA (SEQ ID NO: 3198) |
| | | | | | GGAAATCATGACCCGAGAGGTGCAG (SEQ ID NO: 3199) |
| | | | | | GACCCGAGAGGTGCAGACAAATGAC (SEQ ID NO: 3200) |
| | | | | | GAGAGGTGCAGACAAATGACTTGAA (SEQ ID NO: 3201) |
| 213098_at | RQCD1 | RCD1; CNOT9; | RCD1 required for | 9215 | ACAGCTCAGGTTTTATCACCGACTG (SEQ ID NO: 3202) |
| | | RCD1+ | cell differentiation1 | | ACCTCAATGCTGAACCGCACTGGAG (SEQ ID NO: 3203) |
| | | | homolog (S. pombe) | | CAAGGTACCCCTGCTGAGGTGTATG (SEQ ID NO: 3204) |
| | | | | | CCCTCTGCTACTCCCAGGAAATGGG (SEQ ID NO: 3205) |
| | | | | | ACCACAGCCCCAGGAGGGTGTCAAC (SEQ ID NO: 3206) |
| | | | | | ATAGCAGCAGCACTCTAGGCATGGT (SEQ ID NO: 3207) |
| | | | | | GCATGGTGAACGCCTGGGACCAAGC (SEQ ID NO: 3208) |
| | | | | | GGGACCAAGCCATGTGGCGTTTTTT (SEQ ID NO: 3209) |
| | | | | | AAGATATGTCTCTTCATTCTCTCTC (SEQ ID NO: 3210) |
| | | | | | TCATTCTCTCTCAGTATTTGTTTAC (SEQ ID NO: 3211) |
| | | | | | GCAAAACTTTGTCGACTGGCACTGT (SEQ ID NO: 3212) |
| 34408_at | RTN2 | NSP2, NSPL1 | reticulon 2 | 6253 | TCCGAGCTAAAATCCCAGGGACCGG (SEQ ID NO: 3213) |
| | | | | | TTACCTGAGCGACCAGGACTACATT (SEQ ID NO: 3214) |
| | | | | | GCCTGCTGGGACTTGTAGTTGCCTA (SEQ ID NO: 3215) |
| | | | | | TGCTGGGACTTGTAGTTGCCTAGAC (SEQ ID NO: 3216) |
| | | | | | TGGGACTTGTAGTTGCCTAGACAGG (SEQ ID NO: 3217) |
| | | | | | TGTAGTTGCCTAGACAGGGCACCAC (SEQ ID NO: 3218) |
| | | | | | GTAGTTGCCTAGACAGGGCACCACC (SEQ ID NO: 3219) |
| | | | | | AGGCGTTGGTGTCTCCTGGATGCTA (SEQ ID NO: 3220) |
| | | | | | GGCGTTGGTGTCTCCTGGATGCTAC (SEQ ID NO: 3221) |
| | | | | | GCGTTGGTGTCTCCTGGATGCTACT (SEQ ID NO: 3222) |
| | | | | | CGTTGGTGTCTCCTGGATGCTACTA (SEQ ID NO: 3223) |
| | | | | | GGGAGGCCTGAGCTTGGATTTACAC (SEQ ID NO: 3224) |
| | | | | | GGAGGCCTGAGCTTGGATTTACACT (SEQ ID NO: 3225) |
| | | | | | GGCCTGAGCTTGGATTTACACTGTA (SEQ ID NO: 3226) |
| | | | | | CTGAGCTTGGATTTACACTGTAATA (SEQ ID NO: 3227) |
| | | | | | CTTGGATTTACACTGTAATAAAGAC (SEQ ID NO: 3228) |
| 216976_s_at | RYK | D3S3195, JTK5, | RYK receptor-like | 6259 | AGGCCAGTTCGTTGGATGGCTCTTG (SEQ ID NO: 3229) |
| | | JTK5A, RYK1 | tyrosine kinase | | CTCTAGCGCTAGTGATGTGTGGGCC (SEQ ID NO: 3230) |
| | | | | | TGGGAACTCATGACTCTGGGCCAGA (SEQ ID NO: 3231) |
| | | | | | ATGGTTACCGAATAGCCCAGCCAAT (SEQ ID NO: 3232) |
| | | | | | CCCAGCCAATCAACTGTCCTGATGA (SEQ ID NO: 3233) |
| | | | | | TATTTGCTGTGATGGCCTGTTGCTG (SEQ ID NO: 3234) |
| | | | | | CCTGTTGCTGGGCCTTAGATCCAGA (SEQ ID NO: 3235) |
| | | | | | GTTTCAGCAGCTGGTACAGTGCCTA (SEQ ID NO: 3236) |
| | | | | | GCCTAACAGAGTTTCATGCAGCCCT (SEQ ID NO: 3237) |
| | | | | | AGGAAGAAGGTGCCTGTCGGGGCTC (SEQ ID NO: 3238) |
| | | | | | TCGGGGCTCACTTGAAGCCTGTCAG (SEQ ID NO: 3239) |
| 218276_s_at | SAV1 | SAV, WW45, | salvador homolog 1 | 60485 | AGTTGCTAACATCATCTTCCTTATT (SEQ ID NO: 3240) |
| | | WWP4 | (Drosophila) | | TTCAGCCAGTGCTGTTACTGCATTT (SEQ ID NO: 3241) |
| | | | | | GTTACTGCATTTAGCATGGCTCTGA (SEQ ID NO: 3242) |
| | | | | | GCATGGCTCTGACGAGGCAGTGGAG (SEQ ID NO: 3243) |
| | | | | | GCAGTGGAGAGTTCCTGACAGCATA (SEQ ID NO: 3244) |
| | | | | | GACAGCATAGCATACTGCCTTCTGA (SEQ ID NO: 3245) |
| | | | | | AGCATACTGCCTTCTGACAGACTGG (SEQ ID NO: 3246) |
| | | | | | GACAGACTGGGTTAAGCTACCAAAA (SEQ ID NO: 3247) |
| | | | | | GACTTTAACACCATATATTTCCCTC (SEQ ID NO: 3248) |
| | | | | | GTCTTCCAGAATACAAACTACTCCC (SEQ ID NO: 3249) |
| | | | | | ACTACTCCCAGTTAGATATCAGTGA (SEQ ID NO: 3250) |
| 212416_at | SCAMP1 | SCAMP, | secretory carrier | 9522 | TTAGAACCCACATATCTTTTTTTGT (SEQ ID NO: 3251) |
| | | SCAMP37 | membrane protein 1 | | GATTGGCATTTGTTAAGTGTTCTAT (SEQ ID NO: 3252) |
| | | | | | TAAAATGGTCTTCACACAGCAGTCA (SEQ ID NO: 3253) |
| | | | | | TTCACACAGCAGTCATCCGTGTCAT (SEQ ID NO: 3254) |
| | | | | | GTCATCCGTGTCATTTATCATTTTG (SEQ ID NO: 3255) |
| | | | | | GGGACTTGGATTATTTATCTAGAGA (SEQ ID NO: 3256) |
| | | | | | TAACTAATACTGCGCTGCCATCATG (SEQ ID NO: 3257) |
| | | | | | GCGCTGCCATCATGGTGACTGTCAT (SEQ ID NO: 3258) |
| | | | | | GTCATGGTTCTACAGAAATGCCCTC (SEQ ID NO: 3259) |
| | | | | | TCCATGTGTCCCTCTAATGTTGCAT (SEQ ID NO: 3260) |
| | | | | | AATGTTGCATGTTTCAGTGGGTTGG (SEQ ID NO: 3261) |
| 204035_at | SCG2 | CHGC, SN, SgII | secretogranin II | 7857 | CCCGGTGAGCAAAAGGTTCCCTGTG (SEQ ID NO: 3262) |
| | | | (chromogranin C) | | TCCCTGTGGGGCCCCCGAAGAATGA (SEQ ID NO: 3263) |
| | | | | | ATGATACCCCAAATAGGCAGTACTG (SEQ ID NO: 3264) |
| | | | | | GAAAGTGCTGGAATACCTCAATCAA (SEQ ID NO: 3265) |
| | | | | | CATTAATTACCCTACTTTCATTCCT (SEQ ID NO: 3266) |
| | | | | | AGTGTGTTGACTTCTATCCTGTTAA (SEQ ID NO: 3267) |
| | | | | | CAGGCAGATGAAACCAGGTCACTGG (SEQ ID NO: 3268) |
| | | | | | CTGAGCTGTTATCTTGTGTATGGAT (SEQ ID NO: 3269) |
| | | | | | GTGTAAATGTTATGACTCCTTGATA (SEQ ID NO: 3270) |
| | | | | | GCTGTGGCATTGTTGATGCTCACAT (SEQ ID NO: 3271) |
| | | | | | GTTACTGTCTGTAGCGTTTTGTGGA (SEQ ID NO: 3272) |
| 212698_s_at | SEPT10 | FLJ11619 | septin 10 | 151011 | GATCTTAAGCTTTTGTGTCAGATTA (SEQ ID NO: 3273) |
| | | | | | TGATGACTTCATGCTTTATTATGCC (SEQ ID NO: 3274) |
| | | | | | TATGCCTTATTATGGCTGACGTATT (SEQ ID NO: 3275) |
| | | | | | GGCTGACGTATTACTGTGGTGAAAC (SEQ ID NO: 3276) |
| | | | | | ATAAAGTACCTTTGAGCATGAGTGT (SEQ ID NO: 3277) |
| | | | | | GCATGAGTGTATCACAGCTTTCATT (SEQ ID NO: 3278) |
| | | | | | GGAAAACTTTTCATTACATACTTGT (SEQ ID NO: 3279) |
| | | | | | ACTTGTTTAAACTCTGTCTTCCAGG (SEQ ID NO: 3280) |
| | | | | | TTCAGGTGGTATTTGCATTCAGTGC (SEQ ID NO: 3281) |
| | | | | | AGTGCCTTACTGGTATTCTCAGAAC (SEQ ID NO: 3282) |
| | | | | | AATAAAACTGACATTTACTCTTGAC (SEQ ID NO: 3283) |
| 214720_x_at | SEPT10 | FLJ11619 | septin 10 | 151011 | GCTACAGGCCAAATTTGAGCACCTT (SEQ ID NO: 3284) |
| | | | | | GAGCACCTTAAGAGACTTCACCAAG (SEQ ID NO: 3285) |
| | | | | | CTACCTCCGAGATATTTCACAGCCA (SEQ ID NO: 3286) |
| | | | | | TTCACAGCCAGTCCTTTCTGGCAAC (SEQ ID NO: 3287) |
| | | | | | GTCCTTTCTGGCAACAGGCAGCAAC (SEQ ID NO: 3288) |
| | | | | | GACAAGGACCGTAAGAACTCCAATT (SEQ ID NO: 3289) |
| | | | | | GAGCACAGAAGGTCATCATCACAAG (SEQ ID NO: 3290) |
| | | | | | GCTGTTATTTGTTTTATCACTTCTA (SEQ ID NO: 3291) |
| | | | | | GGTGAACAGCCACAGTTACTGATAT (SEQ ID NO: 3292) |
| | | | | | GATACTACAAGTTGAGCATCTCTAA (SEQ ID NO: 3293) |
| | | | | | TCTTTTTGAGCACTGACTTGACCCC (SEQ ID NO: 3294) |
| 217724_at | SERBP1 | CGI-55, CHD3IP, | SERPINE1 mRNA | 26135 | AGTTACCTACTATTCTGACATTTTA (SEQ ID NO: 3295) |
| | | DKFZp564M2423, | binding protein 1 | | CTTGTGCTGGTGTTTTGGATCTTAT (SEQ ID NO: 3296) |
| | | FLJ90489, | | | GATGCTGAGCATGTTCTGCACTGGT (SEQ ID NO: 3297) |
| | | HABP4L, PAI- | | | TTTACACACATACGTGCTACCCAGA (SEQ ID NO: 3298) |
| | | RBP1, PAIRBP1 | | | AGACAGCAACATACGCACTCCTAAA (SEQ ID NO: 3299) |
| | | | | | AAGTATCTAACTCATTTCCAAACAT (SEQ ID NO: 3300) |
| | | | | | TAAACCTCTTGATTTCCAGCAACAT (SEQ ID NO: 3301) |
| | | | | | AACACCTGCTACTCAAAACACAACT (SEQ ID NO: 3302) |
| | | | | | ATATCTGGTATGTTGCAAGCTTTCA (SEQ ID NO: 3303) |
| | | | | | ATAAATTTGCCTCCTAGTTCACTTT (SEQ ID NO: 3304) |
| | | | | | TGAACCTAACTTTCTCTTATGGTG (SEQ ID NO: 3305) |
| 202833_s_at | SERPINA1 | A1A, A1AT, AAT, | serpin peptidase | 5265 | GAAGCGTTTAGGCATGTTTAACATC (SEQ ID NO: 3306) |
| | | MGC23330, | inhibitor, clade A | | GCATGTTTAACATCCAGCACTGTAA (SEQ ID NO: 3307) |
| | | MGC9222, PI, | (alpha-1 | | AGCACTGTAAGAAGCTGTCCAGCTG (SEQ ID NO: 3308) |
| | | PI1, PRO2275 | antiproteinase, | | GAGGGGAAACTACAGCACCTGGAAA (SEQ ID NO: 3309) |
| | | | antitrypsin), member 1 | | CACCTGGAAAATGAACTCACCCACG (SEQ ID NO: 3310) |
| | | | | | ATGAACTCACCCACGATATCATCAC (SEQ ID NO: 3311) |
| | | | | | CCCACGATATCATCACCAAGTTCCT (SEQ ID NO: 3312) |
| | | | | | GAAGACAGAAGGTCTGCCAGCTTAC (SEQ ID NO: 3313) |
| | | | | | GAAGGTCTGCCAGCTTACATTTACC (SEQ ID NO: 3314) |
| | | | | | GCTTACATTTACCCAAACTGTCCAT (SEQ ID NO: 3315) |
| | | | | | TTACCCAAACTGTCCATTACTGGAA (SEQ ID NO: 3316) |
| 211429_s_at | SERPINA1 | A1A, A1AT, AAT, | serpin peptidase | 5265 | TACTGGAACCTATGATCTGAAGAGC (SEQ ID NO: 3317) |
| | | MGC23330, | inhibitor, clade A | | ACCTATGATCTGAAGAGCGTCCTGG (SEQ ID NO: 3318) |
| | | MGC9222, PI, | (alpha-1 | | TGGGTCAACTGGGCATCACTAAGGT (SEQ ID NO: 3319) |
| | | PI1, PRO2275 | antiproteinase, | | GGTCAACTGGGCATCACTAAGGTCT (SEQ ID NO: 3320) |
| | | | antitrypsin), member 1 | | GGCCGTGCATAAGGCTGTGCTGACC (SEQ ID NO: 3321) |
| | | | | | TGCATAAGGCTGTGCTGACCATCGA (SEQ ID NO: 3322) |
| | | | | | AAACCCTTTGTCTTCTTAATGATTG (SEQ ID NO: 3323) |
| | | | | | CCTTTGTCTTCTTAATGATTGAACA (SEQ ID NO: 3324) |
| | | | | | TGAACAAAATACCAAGTCTCCCCTC (SEQ ID NO: 3325) |
| | | | | | CACCCAAAAATAACTGCCTCTCGCT (SEQ ID NO: 3326) |
| | | | | | CCCCCTCCCTGGATGACATTAAAGA (SEQ ID NO: 3327) |
| 201070_x_at | SF3B1 | PRP10, PRPF10, | splicing factor 3b, | 23451 | TGGATGGGCTGAGACTCCTCGAACA (SEQ ID NO: 3328) |
| | | SAP155, | subunit 1, 155 kDa | | TGGGTGGAAGCACTCCAGTTCTGAC (SEQ ID NO: 3329) |
| | | SF3b155 | | | AGTTCTGACCCCTGGAAAGACACCA (SEQ ID NO: 3330) |
| | | | | | AAAGACACCAATTGGCACACCAGCC (SEQ ID NO: 3331) |
| | | | | | ACCCCTACTCCAGGTCACATAATGA (SEQ ID NO: 3332) |
| | | | | | TGAACAGCTTCAGGCTTGGCGGTGG (SEQ ID NO: 3333) |
| | | | | | AAATCGCCCACTTTCTGATGAGGAA (SEQ ID NO: 3334) |
| | | | | | AGGATATAAGGTACTTCCTCCTCCA (SEQ ID NO: 3335) |
| | | | | | TCCTCCAGCTGGTTATGTTCCTATT (SEQ ID NO: 3336) |
| | | | | | GTTCCTATTCGAACTCCAGCTCGAA (SEQ ID NO: 3337) |
| | | | | | CAGCTACTCCAACACCTTTGGGTGG (SEQ ID NO: 3338) |
| 201586_s_at | SFPQ | POMP100, PSF | splicing factor | 6421 | GCCATTCATGCTTCTATGCATATTA (SEQ ID NO: 3339) |
| | | | proline/glutamine-rich | | ATATTAGGCTACGTATTCCACATTG (SEQ ID NO: 3340) |
| | | | (polypyrimidine tract | | AAGCATGAGAGTGTCTAGGCCTTTG (SEQ ID NO: 3341) |
| | | | binding protein | | TAGGCCTTTGAATGGCATATGCCAT (SEQ ID NO: 3342) |
| | | | associated) | | TCTGGGAAATGCATCTGGAGGCTAA (SEQ ID NO: 3343) |
| | | | | | GGAGGCTAAGTATTGCTTTCTACAA (SEQ ID NO: 3344) |
| | | | | | GTTTGGCATATAGGAAGCACGCTGG (SEQ ID NO: 3345) |
| | | | | | GCACGCTGGTGCTAAGTATTTTTTA (SEQ ID NO: 3346) |
| | | | | | AAATGGATCTTTTATGGCCCTATGA (SEQ ID NO: 3347) |
| | | | | | GCCCTATGATCTATCCTTTACTTGA (SEQ ID NO: 3348) |
| | | | | | AAAGACCAACAAATCTCAAGCCCTA (SEQ ID NO: 3349) |
| 214492_at | SGCD | 35DAG, CMD1L, | sarcoglycan, delta | 6444 | ATCAGGCTACCTAGACTGCCTCATG (SEQ ID NO: 3350) |
| | | DAGD, | (35 kDa dystrophin- | | GATCCTACACGCCTACAGGAACGAG (SEQ ID NO: 3351) |
| | | MGC22567, SG- | associated | | CAGAAGGTCTTCGAGATCTGCGTCT (SEQ ID NO: 3352) |
| | | delta, SGCDP, | glycoprotein) | | TCTGCGTCTGCGCCAATGGGAGATT (SEQ ID NO: 3353) |
| | | SGD | | | GAGCTGGGTCCACTTGTCAGATAAA (SEQ ID NO: 3354) |
| | | | | | AAACACAAGTGTCTGCCTCTGAAAG (SEQ ID NO: 3355) |
| | | | | | CCTTTTTTGGCTTTAGACACTGGCT (SEQ ID NO: 3356) |
| | | | | | ACACTGGCTGCCAGCTATTTTTACT (SEQ ID NO: 3357) |
| | | | | | GTGTGCGTGCTTGAGTGTGTTCGCG (SEQ ID NO: 3358) |
| | | | | | GTGTTCGCGTGTGTGGGTGGATATA (SEQ ID NO: 3359) |
| | | | | | CTGCGATATCCACTGTCATTTTCCA (SEQ ID NO: 3360) |
| 210796_x_at | SIGLEC6 | CD327, CD33L, | sialic acid binding Ig- | 946 | AATCTCTCTGAGTCTCTTTGTGCAT (SEQ ID NO: 3361) |
| | | CD33L1, | like lectin 6 | | CTTTGTGCATTGGTCATCAGCACCA (SEQ ID NO: 3362) |
| | | CDw327, | | | ATCAGCACCAGTTCCAGACAGGCAT (SEQ ID NO: 3363) |
| | | OBBP1, SIGLEC-6 | | | GACAGGCATAGTTTCAGACCACCCT (SEQ ID NO: 3364) |
| | | | | | GTCCTACACTTCCACAAGGTGCAAC (SEQ ID NO: 3365) |
| | | | | | AAAGGTCACCGACACTGAGTACTCA (SEQ ID NO: 3366) |
| | | | | | GAACATGGTACCTCTCAGTGTATTG (SEQ ID NO: 3367) |
| | | | | | CTAAGGCCTCTCTTGTTGGCTTGTA (SEQ ID NO: 3368) |
| | | | | | ACATGGTCTTTCCTCTGAGTGTGTT (SEQ ID NO: 3369) |
| | | | | | TGTCCTAATCTTCTCTTCTTATAAA (SEQ ID NO: 3370) |
| | | | | | ATATTGGATTAGGGCCTCCCCATGA (SEQ ID NO: 3371) |
| 204587_at | SLC25A14 | RP1-20I3.5, | solute carrier family | 9016 | CTGAATTATATGTGAGCCCAGCCCT (SEQ ID NO: 3372) |
| | | BMCP1, | 25 (mitochondrial | | TTTTCCCGTGTTCTAATGTATTTTG (SEQ ID NO: 3373) |
| | | MGC149543, | carrier, brain), | | GTGTTTACCAAGCCGTTGGTCTCCT (SEQ ID NO: 3374) |
| | | UCP5 | member 14 | | TCTCCTAAGGGCCTCCTGATGGAAG (SEQ ID NO: 3375) |
| | | | | | GTGTTAACATTGAGACTCTGGCCCC (SEQ ID NO: 3376) |
| | | | | | TGGCCCCAGATTGGTATCTTCTATG (SEQ ID NO: 3377) |
| | | | | | GGGTGACATTGAAAACGGCCTGCTT (SEQ ID NO: 3378) |
| | | | | | TGAAAACGGCCTGCTTTCCAAATGT (SEQ ID NO: 3379) |
| | | | | | GTAATTGGTTAGCCCCAGACTTGGG (SEQ ID NO: 3380) |
| | | | | | GAAGGCATAGGCCAGGGTGGTTATT (SEQ ID NO: 3381) |
| | | | | | ATGTGTTACAGACCTCGGTTCTCAT (SEQ ID NO: 3382) |
| 200030_s_at | SLC25A3 | D1S155E, | solute carrier family | 5250 | ACACCATGATGAAGTTCGCCTGCTT (SEQ ID NO: 3383) |
| | | MGC165858 | 25 (mitochondrial | | TCGCCTGCTTTGAACGTACTGTTGA (SEQ ID NO: 3384) |
| | | | carrier; phosphate | | TGTACAAGTTTGTGGTTCCTAAGCC (SEQ ID NO: 3385) |
| | | | carrier), member 3 | | TCCTAAGCCCCGCAGTGAATGTTCA (SEQ ID NO: 3386) |
| | | | | | ACCCTGCTGATTCTGTGGTATCTGT (SEQ ID NO: 3387) |
| | | | | | AAAGGTAGCAGTGCTTCTCTGGTCC (SEQ ID NO: 3388) |
| | | | | | GTGCTTCTCTGGTCCTCAAGAGACT (SEQ ID NO: 3389) |
| | | | | | TCATCATGATTGGTACCCTGACTGC (SEQ ID NO: 3390) |
| | | | | | GTACCCTGACTGCACTACAGTGGTT (SEQ ID NO: 3391) |
| | | | | | GTGGTTTATCTATGACTCCGTGAAG (SEQ ID NO: 3392) |
| | | | | | GTGAAGGTCTACTTCAGACTTCCTC (SEQ ID NO: 3393) |
| 209267_s_at | SLC39A8 | BIGM103; LZT- | solute carrier family | 64116 | AATGCAAAACAACAGCTGGACTGCT (SEQ ID NO: 3394) |
| | | Hs6 | 39 (zinc transporter), | | AGCTGGACTGCTGTACATCAAGGAC (SEQ ID NO: 3395) |
| | | | member 8 | | GTTCCTTATGTGTGATTGAGAGCCA (SEQ ID NO: 3396) |
| | | | | | GCCATTCAGAAAAGACTTCCTTTGT (SEQ ID NO: 3397) |
| | | | | | TTGTGTTCAGCCTATACTTTTCCAT (SEQ ID NO: 3398) |
| | | | | | TACTTTTCCATATGGTATACCTTGA (SEQ ID NO: 3399) |
| | | | | | ATTAGCACACCATGGTTATTTTTCT (SEQ ID NO: 3400) |
| | | | | | AAGACAGAGCCTGTTTACTCATTTA (SEQ ID NO: 3401) |
| | | | | | TGAACATCCTAGATTCACACTCCCA (SEQ ID NO: 3402) |
| | | | | | GACAAAGCCCAACAATGATCTCAGG (SEQ ID NO: 3403) |
| | | | | | ATGTTTTCATGTAGCAGCAATGCAG (SEQ ID NO: 3404) |
| 219869_s_at | SLC39A8 | BIGM103; LZT- | solute carrier family | 64116 | GGATGATAACGCTCTGCGATGCCCT (SEQ ID NO: 3405) |
| | | Hs6 | 39 (zinc transporter), | | TGCCCTCCACAATTTCATCGATGGC (SEQ ID NO: 3406) |
| | | | member 8 | | TCGATGGCCTGGCGATTGGGGCTTC (SEQ ID NO: 3407) |
| | | | | | GACTCAGTACTTCCATAGCAATCCT (SEQ ID NO: 3408) |
| | | | | | ATGTGAGGAGTTTCCCCACGAGTTA (SEQ ID NO: 3409) |
| | | | | | GAGACTTTGTGATCCTACTCAATGC (SEQ ID NO: 3410) |
| | | | | | AGGGATGAGCACTCGACAAGCCTTG (SEQ ID NO: 3411) |
| | | | | | ACAAGCCTTGCTATTCAACTTCCTT (SEQ ID NO: 3412) |
| | | | | | TGGTGGGCAACAATTTCGCTCCAAA (SEQ ID NO: 3413) |
| | | | | | GAAAAACCGATTTCACCTTCTTCAT (SEQ ID NO: 3414) |
| | | | | | GCCATTCTACTCATTACCTTGTATG (SEQ ID NO: 3415) |
| 209257_s_at | SMC3 | BAM, BMH, | structural | 9126 | GTAGATCCCTTCAGTTCAAAACATA (SEQ ID NO: 3416) |
| | | CDLS3, CSPG6, | maintenance of | | GAGTTTAGTCATCTTCGTCCAGAAC (SEQ ID NO: 3417) |
| | | HCAP, SMC3L1 | chromosomes 3 | | TCGTCCAGAACAGCGGTTGGCTTTA (SEQ ID NO: 3418) |
| | | | | | ATTGCATGAAGGTACTGGTCCTCGT (SEQ ID NO: 3419) |
| | | | | | AGGTACTGGTCCTCGTGTTATTTCT (SEQ ID NO: 3420) |
| | | | | | AATTCAGACAACCGGTTACCAATCG (SEQ ID NO: 3421) |
| | | | | | ACTTCGAAGAGTTATTGGTGCCAAA (SEQ ID NO: 3422) |
| | | | | | AAATGATGTGATGAACCTCCTTGAA (SEQ ID NO: 3423) |
| | | | | | GAACCTCCTTGAAAGCGCTGGTTTT (SEQ ID NO: 3424) |
| | | | | | GCGCTGGTTTTTCTCGAAGCAATCC (SEQ ID NO: 3425) |
| | | | | | GCAACAGCACCAGATTCTCAGAGAT (SEQ ID NO: 3426) |
| 201793_x_at | SMG7 | RP1-127C7.1, | Smg-7 homolog, | 9887 | CCCCGCCTGTGGCATTTTCTATGGG (SEQ ID NO: 3427) |
| | | C1orf16, EST1C, | nonsense mediated | | TTTCTATGGGCTCAGGTTACACCTT (SEQ ID NO: 3428) |
| | | FLJ23717, | mRNA decay factor | | CAGCTGGTGTTTCTGTCCCAGGAAC (SEQ ID NO: 3429) |
| | | KIAA0250, | (C. elegans) | | CTCACTCTCCAGCAGGAAACCAGGT (SEQ ID NO: 3430) |
| | | SGA56M, SMG-7 | | | GACCAGGGCCAATGAACCAGGGACC (SEQ ID NO: 3431) |
| | | | | | ATCTTTACCAGCTCAGCCAACAGCA (SEQ ID NO: 3432) |
| | | | | | GCCAACAGCACAGTCTACAAGCCAG (SEQ ID NO: 3433) |
| | | | | | GCTGCAGGTTCAAGCTCTAACTCAG (SEQ ID NO: 3434) |
| | | | | | CTAACTCAGCAACAACAATCCCCTA (SEQ ID NO: 3435) |
| | | | | | CCTCACCACTCTGGATTCCAGCAGT (SEQ ID NO: 3436) |
| | | | | | TGTTTGAGCCGTCATTGCAACCTCC (SEQ ID NO: 3437) |
| 205596_s_at | SMURF2 | DKFZp686F0270, | SMAD specific E3 | 64750 | GGTCTTTATGCTTTGCCATGAGGCC (SEQ ID NO: 3438) |
| | | MGC138150 | ubiquitin protein | | GAGGCCACATTCAGCTGCTATTTAA (SEQ ID NO: 3439) |
| | | | ligase 2 | | GAATGCTGACTTTTCCTACATTTCC (SEQ ID NO: 3440) |
| | | | | | TGACTTTTCCTACATTTCCAGAGTT (SEQ ID NO: 3441) |
| | | | | | GAGTTAGGCAGTATTCTACACTTAA (SEQ ID NO: 3442) |
| | | | | | TACACTTAAAGACTACTACTATTTT (SEQ ID NO: 3443) |
| | | | | | TCTTCACAGATTTCAAGTCTCTCAA (SEQ ID NO: 3444) |
| | | | | | AGATTTCAAGTCTCTCAAACATCAA (SEQ ID NO: 3445) |
| | | | | | TCAAGACAACTTCAGCAGTCGGTAC (SEQ ID NO: 3446) |
| | | | | | GCAGTCGGTACAAGTCACATTTCAT (SEQ ID NO: 3447) |
| | | | | | ACAAGTCACATTTCATTTTGATTGA (SEQ ID NO: 3448) |
| 200826_at | SNRPD2 | SMD2, SNRPD1 | small nuclear | 6633 | GAGCGTAGTGACCATCATGAGCCTC (SEQ ID NO: 3449) |
| | | | ribonucleoprotein D2 | | GCCCAAGAGTGAGATGACCCCAGAG (SEQ ID NO: 3450) |
| | | | polypeptide 16.5 kDa | | GAGGAATTTAACACCGGTCCACTCT (SEQ ID NO: 3451) |
| | | | | | CACTCTCTGTGCTCACACAGTCAGT (SEQ ID NO: 3452) |
| | | | | | GAACAATACCCAAGTGCTCATCAAC (SEQ ID NO: 3453) |
| | | | | | TGCTCATCAACTGCCGCAACAATAA (SEQ ID NO: 3454) |
| | | | | | GAAGGCCTTCGATAGGCACTGCAAC (SEQ ID NO: 3455) |
| | | | | | GTGGACTGAGGTACCCAAGAGTGGC (SEQ ID NO: 3456) |
| | | | | | ACATCTCCAAGATGTTCCTGCGCGG (SEQ ID NO: 3457) |
| | | | | | TCATCGCCGGCAAGTAGGGGCCGCC (SEQ ID NO: 3458) |
| | | | | | TCCTATGAAGACCGCTGCCATTGGT (SEQ ID NO: 3459) |
| 201562_s_at | SORD | SORD1 | sorbitol | 6652 | GATGTTAATGGGCTCTGCTCATCCC (SEQ ID NO: 3460) |
| | | | dehydrogenase | | GGGTGGGCTCTGATGCAGAACTTTC (SEQ ID NO: 3461) |
| | | | | | GGAACTTGTAGCCAGAATGCCCTGT (SEQ ID NO: 3462) |
| | | | | | GAATGCCCTGTTCATGCTGAGCAAA (SEQ ID NO: 3463) |
| | | | | | TTCTTCCTGGAGTGCCTTCATTGAG (SEQ ID NO: 3464) |
| | | | | | TCCACTGCTACTGACCCAGAGGGGA (SEQ ID NO: 3465) |
| | | | | | TCATGAAGACTTAACTGGCCCAGAA (SEQ ID NO: 3466) |
| | | | | | AAAATCTGCCACTCAGGGTCTGGGA (SEQ ID NO: 3467) |
| | | | | | GGGTCTGGGATGAAGGCTTGTCAGC (SEQ ID NO: 3468) |
| | | | | | GCTTGTCAGCACTTCCAGTTTAGAA (SEQ ID NO: 3469) |
| | | | | | GAAAAGGCATCACTTTCCTGGATCC (SEQ ID NO: 3470) |
| 201417_at | SOX4 | EVI16 | SRY (sex | 6659 | GTAAACCACATCTTTTTTGCACTTT (SEQ ID NO: 3471) |
| | | | determining region | | ACGTGCCGTTTAAACCACTGGATCT (SEQ ID NO: 3472) |
| | | | Y)-box 4 | | ACCACTGGATCTATCTAAATGCCGA (SEQ ID NO: 3473) |
| | | | | | AATGCCGATTTGAGTTCGCGACACT (SEQ ID NO: 3474) |
| | | | | | GCGACACTATGTACTGCGTTTTTCA (SEQ ID NO: 3475) |
| | | | | | TGTACTGCGTTTTTCATTCTTGTAT (SEQ ID NO: 3476) |
| | | | | | TGACTATTTAATCCTTTCTACTTGT (SEQ ID NO: 3477) |
| | | | | | TCCTTTCTACTTGTCGCTAAATATA (SEQ ID NO: 3478) |
| | | | | | GCACTAGGACGTCTGCCTTTTTAAG (SEQ ID NO: 3479) |
| | | | | | TTTTTTTGCCATCCATCCTGTGCAA (SEQ ID NO: 3480) |
| | | | | | TCCATCCTGTGCAATATGCCGTGTA (SEQ ID NO: 3481) |
| 222163_s_at | SPATA5L1 | FLJ12286, | spermatogenesis | 79029 | GAAGTGTCATGATTATTGCAGCAAC (SEQ ID NO: 3482) |
| | | MGC5347 | associated 5-like 1 | | AGACCTGATGTGTTAGATACTGCTT (SEQ ID NO: 3483) |
| | | | | | AGATACTGCTTTGTTACGACCTGGA (SEQ ID NO: 3484) |
| | | | | | CTCCTTAGAAAACCTCGCAGCAGAA (SEQ ID NO: 3485) |
| | | | | | GCACAGAAGCTGCTTTGCTGGCTCT (SEQ ID NO: 3486) |
| | | | | | CTGCTTTGCTGGCTCTGCAAGAAAA (SEQ ID NO: 3487) |
| | | | | | AATGGACTAGACGCAACTACAGTGA (SEQ ID NO: 3488) |
| | | | | | ACCGTCGTTAAGTTGCAAGGACTTG (SEQ ID NO: 3489) |
| | | | | | AAATCACCTTAAACTCTTGTTCAGT (SEQ ID NO: 3490) |
| | | | | | AAATGTGAACTTGCCTGTCGTTTGC (SEQ ID NO: 3491) |
| | | | | | GTCGTTTGCAACTTCACACTTTTAG (SEQ ID NO: 3492) |
| 220192_x_at | SPDEF | PDEF, RP11- | SAM pointed domain | 25803 | TGAACTACGACAAGCTGAGCCGCTC (SEQ ID NO: 3493) |
| | | 375E1_A.3, | containing ets | | GGGCATCATCCGGAAGCCAGACATC (SEQ ID NO: 3494) |
| | | bA375E1.3 | transcription factor | | GGAAAACGGGCAGTCTGCTCTGCTG (SEQ ID NO: 3495) |
| | | | | | CTGCTCTGACCTTCCAGAGCCCAAG (SEQ ID NO: 3496) |
| | | | | | GCTGCTCCCCTGGAGGACAGAGGGA (SEQ ID NO: 3497) |
| | | | | | AGAGGGAGACAGGGCTGCTCCCCAA (SEQ ID NO: 3498) |
| | | | | | CCAGCATTTCCAGAGCAGAGCCTAC (SEQ ID NO: 3499) |
| | | | | | GGGCAGTGACTCGACAAAGGCCACA (SEQ ID NO: 3500) |
| | | | | | AAAGGCCACAGGCAGTCCAGGCCTC (SEQ ID NO: 3501) |
| | | | | | TGCAGGGAGACATCTGCACCCCTGA (SEQ ID NO: 3502) |
| | | | | | GAGTTGGGCAGCCAGGAGTGCCCCC (SEQ ID NO: 3503) |
| 205064_at | SPRR1B | CORNIFIN, | small proline-rich | 6699 | TACAGAGTATTCCTCTCTTCACACC (SEQ ID NO: 3504) |
| | | GADD33, | protein 1B (cornifin) | | ACCAGCCACTGTTGCAGCATGAGTT (SEQ ID NO: 3505) |
| | | MGC61901, | | | TGTTGCAGCATGAGTTCCCAGCAGC (SEQ ID NO: 3506) |
| | | SPRR1 | | | TCCCATTCTGTGTATGAGTCCCATT (SEQ ID NO: 3507) |
| | | | | | TCTGTGTATGAGTCCCATTTGCCTT (SEQ ID NO: 3508) |
| | | | | | GAGTCCCATTTGCCTTGCAATTAGC (SEQ ID NO: 3509) |
| | | | | | GAATGTGCTATGAAGCTTTCTTTCC (SEQ ID NO: 3510) |
| | | | | | TTTCCTACACACTCTGAGTCTCTGA (SEQ ID NO: 3511) |
| | | | | | GCTGAAGGTCTTAGTACCAGAGCTA (SEQ ID NO: 3512) |
| | | | | | ACCAGAGCTAGTTTTCAGCTGCTCA (SEQ ID NO: 3513) |
| | | | | | GTTTTCAGCTGCTCAGAATTCATCT (SEQ ID NO: 3514) |
| 217995_at | SQRDL | CGI-44 | sulfide quinone | 58472 | TACGTCAAAGACCGCTGCTGCAGTA (SEQ ID NO: 3515) |
| | | | reductase-like (yeast) | | GCAGTAGCTGCCCAGTCAGGAATAC (SEQ ID NO: 3516) |
| | | | | | GCTACACATCATGTCCACTGGTGAC (SEQ ID NO: 3517) |
| | | | | | CACTGGTGACCGGCTACAACCGTGT (SEQ ID NO: 3518) |
| | | | | | ACAACCGTGTGATTCTTGCTGAGTT (SEQ ID NO: 3519) |
| | | | | | GCGCCTTTCCATGTATCTCATGAAA (SEQ ID NO: 3520) |
| | | | | | GACCAGCGTTTCTGCGCAAGTTGTT (SEQ ID NO: 3521) |
| | | | | | TAAGGATGGCTCAGCACTTGCTCAT (SEQ ID NO: 3522) |
| | | | | | GGCTTCTGGGCCAAAACTGCAGTCA (SEQ ID NO: 3523) |
| | | | | | GTAATGGTGACCAAATGCCTCCCTT (SEQ ID NO: 3524) |
| | | | | | GTGGGCTACTCATGATGGGCTTGAT (SEQ ID NO: 3525) |
| 208801_at | SRP72 | | signal recognition | 6731 | GGTCACATGGGTTTGGACTGTCTCA (SEQ ID NO: 3526) |
| | | | particle 72 kDa | | GTATCTGAGTTGCATACCAGGCAGT (SEQ ID NO: 3527) |
| | | | | | CTAACAGGTCTGTTTGGCCCATTGA (SEQ ID NO: 3528) |
| | | | | | CTGTTTGGCCCATTGATAGATACTC (SEQ ID NO: 3529) |
| | | | | | TGGTGTCTCCTTCTGGTTATGGATT (SEQ ID NO: 3530) |
| | | | | | ATTTTGACCATTGATTACCTTTCTC (SEQ ID NO: 3531) |
| | | | | | GTCTTTCCTTGCTTACAAACTACTT (SEQ ID NO: 3532) |
| | | | | | AACTACTTTCACATTGAACAGCTGT (SEQ ID NO: 3533) |
| | | | | | GTATTCATTTTATGCTGCCCAAGAT (SEQ ID NO: 3534) |
| | | | | | GTGATTATATTACTCTGTCCTTGTT (SEQ ID NO: 3535) |
| | | | | | TAAAGTGCTGCTGTGTTTGACTCTG (SEQ ID NO: 3536) |
| 210378_s_at | SSNA1 | N14, NA-14, | Sjogren syndrome | 8636 | AGCGTTCTCAAGAGGGAAGCTGGGA (SEQ ID NO: 3537) |
| | | NA14 | nuclear autoantigen 1 | | TGGGAACCTGACCAAGGCTACAGCC (SEQ ID NO: 3538) |
| | | | | | GACCAGAAAAGTAGCGGCGGCAGGG (SEQ ID NO: 3539) |
| | | | | | GCCCCAGCAAGTGTGTGCTCAGAGC (SEQ ID NO: 3540) |
| | | | | | TGCTCAGAGCATCTTTGTTCTTCAC (SEQ ID NO: 3541) |
| | | | | | CAGGTGCCGAGAGGGGCAGGTGCCA (SEQ ID NO: 3542) |
| | | | | | CTTCATGCTCACACAGGCTATGGGG (SEQ ID NO: 3543) |
| | | | | | CTGCAAGGGGCTTGTCTCTGTGGCA (SEQ ID NO: 3544) |
| | | | | | TGCCAGGGAGGCTCTGGTTGTCTGA (SEQ ID NO: 3545) |
| | | | | | TGGTTGTCTGAGCACCATGGGGGCC (SEQ ID NO: 3546) |
| | | | | | CCTGTGGCCTGTGATTATGAATAAA (SEQ ID NO: 3547) |
| 210242_x_at | ST20 | HCCS-1 | suppressor of | 400410 | GCCATTACCAATCGCGAAACCCGAA (SEQ ID NO: 3548) |
| | | | tumorigenicity 20 | | TGAGGAGGCGGTTCATCTGTGGCCA (SEQ ID NO: 3549) |
| | | | | | GGCCCTGCTATATGCGGGGGCCTCG (SEQ ID NO: 3550) |
| | | | | | CGGCTGGAGTGAGCGGCGTGGACGC (SEQ ID NO: 3551) |
| | | | | | TTCCCTTGTTGCTGCGAGAGCGAGA (SEQ ID NO: 3552) |
| | | | | | TAACGTGAACGCGGACCAGGAGGCT (SEQ ID NO: 3553) |
| | | | | | CTCTGTCTCCCAGGTTCAGGAAAAT (SEQ ID NO: 3554) |
| | | | | | AATCTGGCTGGATGACTTTTCTAGA (SEQ ID NO: 3555) |
| | | | | | TCTGTGAAATGCTCTTCTGTCCTGA (SEQ ID NO: 3556) |
| | | | | | GCCTAATTTTTCTGACTCTTACGAA (SEQ ID NO: 3557) |
| | | | | | TTGCCAACACATACTTCTACTTTTA (SEQ ID NO: 3558) |
| 204068_at | STK3 | FLJ90748, KRS1, | serine/threonine | 6788 | ATAGATCTATCGTGTTTAAAACCAA (SEQ ID NO: 3559) |
| | | MST2 | kinase 3 (STE20 | | GAAATCAATGGAACCCTTGCACAAA (SEQ ID NO: 3560) |
| | | | homolog, yeast) | | AAACTGTAATAAGCTCATCAATTTC (SEQ ID NO: 3561) |
| | | | | | AATGTGCTCTGTTGTCACAGGATGT (SEQ ID NO: 3562) |
| | | | | | ATTTCCTGGGAATTGGTAAACATCA (SEQ ID NO: 3563) |
| | | | | | GTTCCTGATGATAACCCAGTAGCAA (SEQ ID NO: 3564) |
| | | | | | GAGTGGTACAAGCCTTGGGGACTGA (SEQ ID NO: 3565) |
| | | | | | AAAGAAACTCATTTTTACGCTGAAT (SEQ ID NO: 3566) |
| | | | | | GCATTGGGACCAGTCATTTCCTAAG (SEQ ID NO: 3567) |
| | | | | | ATTTCCTAAGCTACATATGGCCATC (SEQ ID NO: 3568) |
| | | | | | TATGGCCATCTTGACAGTGTTTTTT (SEQ ID NO: 3569) |
| 204287_at | SYNGR1 | MGC: 1939 | synaptogyrin 1 | 9145 | TGAGGGGCTAATTCCCAGGGGACTG (SEQ ID NO: 3570) |
| | | | | | CTTCCCTGGTGATTCTTGATCTTCA (SEQ ID NO: 3571) |
| | | | | | TCAAAGCCTCGGGGATCCCAGGTTT (SEQ ID NO: 3572) |
| | | | | | GGTTTCCCCATGTAACTGAGACACC (SEQ ID NO: 3573) |
| | | | | | CCCCTTTGATTCTCTAAGGAGCACG (SEQ ID NO: 3574) |
| | | | | | ATGCCCCTCCCAGGAGTTTATGGGA (SEQ ID NO: 3575) |
| | | | | | GCAGCGTTACTGCCTGTGTATAGTA (SEQ ID NO: 3576) |
| | | | | | CGTGTGTGTGCGGTGAAGGGCGGTC (SEQ ID NO: 3577) |
| | | | | | CCCACCACCTGGTTAAGTCTCGAGT (SEQ ID NO: 3578) |
| | | | | | AAGTCTCGAGTGAATCCAGTGGCCC (SEQ ID NO: 3579) |
| | | | | | GACATCCATCCATCTGTGGTGAACC (SEQ ID NO: 3580) |
| 218746_at | TAPBPL | TAPBPR; | TAP binding protein- | 55080 | CTCATCTGCGACATTGCTGGCTATT (SEQ ID NO: 3581) |
| | | TAPBP-R; | like | | TCTGCGACATTGCTGGCTATTACCC (SEQ ID NO: 3582) |
| | | FLJ10143 | | | TGGTGACGTGGACCCGAGAGGAGCT (SEQ ID NO: 3583) |
| | | | | | CGTGGACCCGAGAGGAGCTGGGTGG (SEQ ID NO: 3584) |
| | | | | | TCTCCAGCCTCAGGCAAAGCGTGGC (SEQ ID NO: 3585) |
| | | | | | GCCTCAGGCAAAGCGTGGCAGGCAC (SEQ ID NO: 3586) |
| | | | | | GCAGGTGCCACTTACACCTGCCAGG (SEQ ID NO: 3587) |
| | | | | | CACTTACACCTGCCAGGTCACACAC (SEQ ID NO: 3588) |
| | | | | | CCATGAAGACCGCACAGCGCGTGTA (SEQ ID NO: 3589) |
| | | | | | CCAGCTGACCTAAAGCGACATGAGA (SEQ ID NO: 3590) |
| | | | | | AACAACAACCAAGCCAGTTTAATGG (SEQ ID NO: 3591) |
| 218747_s_at | TAPBPL | TAPBPR; | TAP binding protein- | 55080 | GTCACACACATCTCTCTGGAGGAGC (SEQ ID NO: 3592) |
| | | TAPBP-R; | like | | GAGCGGAGAACAGCCTTGGGAGTCA (SEQ ID NO: 3593) |
| | | FLJ10143 | | | GAACAGCCTTGGGAGTCATCTTTGC (SEQ ID NO: 3594) |
| | | | | | AGCCTTGGGAGTCATCTTTGCCAGC (SEQ ID NO: 3595) |
| | | | | | GGGGCTTCAGAGACGGCAAGCACCT (SEQ ID NO: 3596) |
| | | | | | CAGAGACGGCAAGCACCTACAGGAC (SEQ ID NO: 3597) |
| | | | | | AGCACCTACAGGACTTGGGCTGCTT (SEQ ID NO: 3598) |
| | | | | | GCTTCAGGCTGAACGCTGGGAGACC (SEQ ID NO: 3599) |
| | | | | | CAGGCTGAACGCTGGGAGACCACTT (SEQ ID NO: 3600) |
| | | | | | AAGCGACATGAGACTACTAGAAAGA (SEQ ID NO: 3601) |
| | | | | | GACTACTAGAAAGAAACGACACCCT (SEQ ID NO: 3602) |
| 200803_s_at | TEGT | BI-1, TMBIM6 | testis enhanced gene | 7009 | CTTGCTGGATTTGCATTCCTTACAG (SEQ ID NO: 3603) |
| | | | transcript (BAX | | CCCTGCCCTGGAGTTTTGTATTGCT (SEQ ID NO: 3604) |
| | | | inhibitor 1) | | TGTATTGCTGTCAACCCCAGCATCC (SEQ ID NO: 3605) |
| | | | | | TCCCACTGCTTTCATGGGCACAGCA (SEQ ID NO: 3606) |
| | | | | | AGCAATGATCTTTACCTGCTTCACC (SEQ ID NO: 3607) |
| | | | | | TGATGTCAGCCCTGAGCTTGTTGCT (SEQ ID NO: 3608) |
| | | | | | GGGACTGGTGGTCATGTGTGGCTTC (SEQ ID NO: 3609) |
| | | | | | TATCTGGCACTGCATTGATCTCTTC (SEQ ID NO: 3610) |
| | | | | | GAAAACTCATGATGATCCTGGCCAT (SEQ ID NO: 3611) |
| | | | | | AAATGAAGTGACCATCCAGCCTTTC (SEQ ID NO: 3612) |
| | | | | | ATTTCCTTTTTGCACACATTACAGG (SEQ ID NO: 3613) |
| 200804_at | TEGT | BI-1, TMBIM6 | testis enhanced gene | 7009 | GACAGCTACCCCCAGAAGGGTCAAT (SEQ ID NO: 3614) |
| | | | transcript (BAX | | GTTGGGAGTGGTTGTGGCTCTGAGC (SEQ ID NO: 3615) |
| | | | inhibitor 1) | | TGCTCTACAGAGCTTCAGTGTGAGA (SEQ ID NO: 3616) |
| | | | | | TTCACAACCAGTGCACAGTTTGACA (SEQ ID NO: 3617) |
| | | | | | TTGACAGTGGCCTCAGGTTCACAGT (SEQ ID NO: 3618) |
| | | | | | AGTGCACCATGTCACTGTGCTATCC (SEQ ID NO: 3619) |
| | | | | | CCCGAATGGCTCACTTTCACTGAGG (SEQ ID NO: 3620) |
| | | | | | TGCTGTCCTCTGATTTAGCTGCTGC (SEQ ID NO: 3621) |
| | | | | | CAGCCTCTGGCTTGAGAACTTACTA (SEQ ID NO: 3622) |
| | | | | | GTGATTCTCTGCTAGGCCTAAGATT (SEQ ID NO: 3623) |
| | | | | | AACATCTCTTGAAGCCAAACTCCAC (SEQ ID NO: 3624) |
| 202720_at | TES | DKFZp586B2022, | testis derived | 26136 | GTATAGATGATTTTTGGCTTGGACA (SEQ ID NO: 3625) |
| | | MGC1146, | transcript (3 LIM | | GGACATAAAAGCCAAGCCACCCATT (SEQ ID NO: 3626) |
| | | TESS, TESS-2 | domains) | | AGCCACCCATTTGCTTTTAATCCAA (SEQ ID NO: 3627) |
| | | | | | GAACATGTATAGTTTTTGTACCCAG (SEQ ID NO: 3628) |
| | | | | | TATTGATTGCACTTGCCTGCCATGA (SEQ ID NO: 3629) |
| | | | | | GCACTTGCCTGCCATGATTTAGATA (SEQ ID NO: 3630) |
| | | | | | TATTCTTTCCTAATGGATCCTGTTT (SEQ ID NO: 3631) |
| | | | | | ATACTTCCAAGCCTGTCCATGGATA (SEQ ID NO: 3632) |
| | | | | | TGTCTTCACTTGTATGTTTTCATGG (SEQ ID NO: 3633) |
| | | | | | ATGTTTTCATGGCTAGGTATTTCTA (SEQ ID NO: 3634) |
| | | | | | TGTACTTCTACACATAGCTATGCAC (SEQ ID NO: 3635) |
| 218099_at | TEX2 | DKFZp781G0721, | testis expressed 2 | 55852 | TGACAGGATGGGTCCTCTCATACAG (SEQ ID NO: 3636) |
| | | HT008, | | | TTTTTCCATCTGGCGTTTCTGTGTC (SEQ ID NO: 3637) |
| | | KIAA1738, | | | GTTTCTGTGTCCTCCAGGTTTATAT (SEQ ID NO: 3638) |
| | | TMEM96 | | | GGGAGAGTTCCATGGGCAGATTTCC (SEQ ID NO: 3639) |
| | | | | | GAAGGCCAAAACGGAGAACTGCTCT (SEQ ID NO: 3640) |
| | | | | | AGACCAAAAGTTTGCTCAGCATCAC (SEQ ID NO: 3641) |
| | | | | | TCAGCATCACACTACATCTCAAAAT (SEQ ID NO: 3642) |
| | | | | | TAGTTTACAAGGTTGGGGGCTCTCT (SEQ ID NO: 3643) |
| | | | | | GGGCTCTCTTTGCTTCGAGAAGTAA (SEQ ID NO: 3644) |
| | | | | | GCTGCATTCAACGTCAAAATTACCT (SEQ ID NO: 3645) |
| | | | | | GCACCTTGCCTGAACATGACTTTAA (SEQ ID NO: 3646) |
| 213135_at | TIAM1 | FLJ36302 | T-cell lymphoma | 7074 | GTTCAGTACATCATGCTCTTGTGCC (SEQ ID NO: 3647) |
| | | | invasion and | | TTTCCAGGGCTTCGAGCTTGATCTT (SEQ ID NO: 3648) |
| | | | metastasis 1 | | TTGCTATATCTTCTGGTTTTCTGAA (SEQ ID NO: 3649) |
| | | | | | ATACCCTTTGTATCACTGCATTTTT (SEQ ID NO: 3650) |
| | | | | | CATTTTTCCATATCATCTCCGGTTC (SEQ ID NO: 3651) |
| | | | | | GGTTCGATCGCGTCCAGATGGAAAA (SEQ ID NO: 3652) |
| | | | | | AGCAGAGGCTTCTAATCGTCGCATT (SEQ ID NO: 3653) |
| | | | | | CGTCGCATTTACTGGCTCCAGTGCA (SEQ ID NO: 3654) |
| | | | | | CTCCAGTGCAACACATCCATCTGAA (SEQ ID NO: 3655) |
| | | | | | AACACTCGGAAGTCTGGTGCTTGGA (SEQ ID NO: 3656) |
| | | | | | GAGAGGGTGCCATTGTCTCTTGTAC (SEQ ID NO: 3657) |
| 219258_at | TIPIN | FLJ20516 | TIMELESS | 54962 | GAACATGATGTCACTTCTACTGAAT (SEQ ID NO: 3658) |
| | | | interacting protein | | CTACTGAATTAGATCCCTTTCTGAC (SEQ ID NO: 3659) |
| | | | | | GATCCCTTTCTGACAAACTTATCTG (SEQ ID NO: 3660) |
| | | | | | GTTAATGAATACACCCAGGGCACAC (SEQ ID NO: 3661) |
| | | | | | CCCAGGGCACACACGGTTGAAGAGG (SEQ ID NO: 3662) |
| | | | | | AACGAAGACATTCTGGACAATCCAT (SEQ ID NO: 3663) |
| | | | | | GAAACACTGCTGGACCAGTCTTTTA (SEQ ID NO: 3664) |
| | | | | | GCAACAGCAACTTGATGCTACATCC (SEQ ID NO: 3665) |
| | | | | | TGTATCTGTTAAGTCATCGTCCTGC (SEQ ID NO: 3666) |
| | | | | | TTAAGTCATCGTCCTGCAAGCTTGG (SEQ ID NO: 3667) |
| | | | | | GCAAGCTTGGCGTTACTATGTATTT (SEQ ID NO: 3668) |
| 203254_s_at | TLN1 | ILWEQ, | talin 1 | 7094 | GAAGATGGTTGGCGGCATTGCCCAG (SEQ ID NO: 3669) |
| | | KIAA1027, TLN | | | TGGTTGGCGGCATTGCCCAGATCAT (SEQ ID NO: 3670) |
| | | | | | TGCCCAGATCATCGCAGCACAGGAA (SEQ ID NO: 3671) |
| | | | | | AATGCTTCGGAAGGAACGAGAGCTG (SEQ ID NO: 3672) |
| | | | | | GAAGAGGCGCGGAAGAAACTGGCCC (SEQ ID NO: 3673) |
| | | | | | CCGGCAGCAGCAGTACAAGTTTCTG (SEQ ID NO: 3674) |
| | | | | | GTACAAGTTTCTGCCTTCAGAGCTT (SEQ ID NO: 3675) |
| | | | | | GAGCTTCGAGATGAGCACTAAAGAA (SEQ ID NO: 3676) |
| | | | | | CTTCTATTTAATGCAGACCCGGCCC (SEQ ID NO: 3677) |
| | | | | | CTCCAAGAGTATTATTAACGCTGCT (SEQ ID NO: 3678) |
| | | | | | TTATTAACGCTGCTGTACCTCGATC (SEQ ID NO: 3679) |
| 209387_s_at | TM4SF1 | H-L6, L6, M3S1, | transmembrane 4 L | 4071 | TTGGCTCTTGGTGGAATTGAATTCA (SEQ ID NO: 3680) |
| | | TAAL6 | six family member 1 | | GAATTGAATTCATCTTGTGTCTTAT (SEQ ID NO: 3681) |
| | | | | | ATTCATCTTGTGTCTTATTCAAGTA (SEQ ID NO: 3682) |
| | | | | | GTAATAAATGGAGTGCTTGGAGGCA (SEQ ID NO: 3683) |
| | | | | | GGAGTGCTTGGAGGCATATGTGGCT (SEQ ID NO: 3684) |
| | | | | | GAGGCATATGTGGCTTTTGCTGCTC (SEQ ID NO: 3685) |
| | | | | | CTTTTGCTGCTCTCACCAACAGCAA (SEQ ID NO: 3686) |
| | | | | | TCCCCACAGTCTACTTTTACAAACG (SEQ ID NO: 3687) |
| | | | | | TTTACAGACTGAGTGACAGTACTCA (SEQ ID NO: 3688) |
| | | | | | GAGTGACAGTACTCAGTATATCTGA (SEQ ID NO: 3689) |
| | | | | | TATATCTGAGATAAACTCTATAATG (SEQ ID NO: 3690) |
| 215033_at | TM4SF1 | H-L6, L6, M3S1, | transmembrane 4 L | 4071 | AAGGTTACCCACATTGAGCTCATAC (SEQ ID NO: 3691) |
| | | TAAL6 | six family member 1 | | GAGCTCATACCAGTGTGAAACTTCC (SEQ ID NO: 3692) |
| | | | | | GAAACTTCCATGGCATTATCTTTTA (SEQ ID NO: 3693) |
| | | | | | ACCCCACACAAATGCACTCAAAGCA (SEQ ID NO: 3694) |
| | | | | | AAAGCAACCCAACATCAGTATCCTG (SEQ ID NO: 3695) |
| | | | | | TGCAGTTTCTATGGGCCCTTTCTGC (SEQ ID NO: 3696) |
| | | | | | TTCTGCAGCCTTTACTCATGGAAGA (SEQ ID NO: 3697) |
| | | | | | AGCATTGTCCAAGCCCTAAGTATGG (SEQ ID NO: 3698) |
| | | | | | GTATGGTTGGCCTAAGCTTTGCTGA (SEQ ID NO: 3699) |
| | | | | | AGATCAGTCTGTTTATGTTTGCTTA (SEQ ID NO: 3700) |
| | | | | | AATGTCATCGTTATTTATCCTGATT (SEQ ID NO: 3701) |
| 215034_s_at | TM4SF1 | H-L6, L6, M3S1, | transmembrane 4 L | 4071 | TATGACTGCTAAAAGAACCAACCCA (SEQ ID NO: 3702) |
| | | TAAL6 | six family member 1 | | AAAGAACCAACCCAGGACAGAGCCA (SEQ ID NO: 3703) |
| | | | | | ACCCAGGACAGAGCCACAATCTTCC (SEQ ID NO: 3704) |
| | | | | | CAGGACAGAGCCACAATCTTCCTCT (SEQ ID NO: 3705) |
| | | | | | ACAGAGCCACAATCTTCCTCTATTT (SEQ ID NO: 3706) |
| | | | | | TTTCACTTGTATTCATTTGTAAAAC (SEQ ID NO: 3707) |
| | | | | | TTTGTATTAGTGTAACATACTCCCC (SEQ ID NO: 3708) |
| | | | | | AAACGCCTGTAAAGACTGGCATCTT (SEQ ID NO: 3709) |
| | | | | | GCCTGTAAAGACTGGCATCTTCACA (SEQ ID NO: 3710) |
| | | | | | GACTGGCATCTTCACAGGATGTCAG (SEQ ID NO: 3711) |
| | | | | | ATCTTCACAGGATGTCAGTGTTTAA (SEQ ID NO: 3712) |
| 213349_at | TMCC1 | DKFZp686M0169, | transmembrane and | 23023 | GATGAGGGCAAACCAGTTAAACCTA (SEQ ID NO: 3713) |
| | | FLJ42680 | coiled-coil domain | | TCCCTATCCCAGACTAACTTTACTG (SEQ ID NO: 3714) |
| | | | family 1 | | TGGATTCGGGGTGGATTTTCGTTGT (SEQ ID NO: 3715) |
| | | | | | GTGGATTTTCGTTGTCCGTGGAAGA (SEQ ID NO: 3716) |
| | | | | | GGAAGAACACATGGATCTCTCTGGC (SEQ ID NO: 3717) |
| | | | | | CTGCCCCTTAACCTTGACGAGGATA (SEQ ID NO: 3718) |
| | | | | | CCCCCAAAGGATCACTGCACAGTCC (SEQ ID NO: 3719) |
| | | | | | ATCACTGCACAGTCCTACTACAGTA (SEQ ID NO: 3720) |
| | | | | | ATATGTGTATAGTTACCAACCTAAA (SEQ ID NO: 3721) |
| | | | | | AAAATCCGAACAGCATACTTGAAGA (SEQ ID NO: 3722) |
| | | | | | AATACTCAAACTCTCAGTGCTTCCT (SEQ ID NO: 3723) |
| 201764_at | TMEM106C | MGC111210, | transmembrane | 79022 | CTACCCCCACGTGGTGTAAGCAGAG (SEQ ID NO: 3724) |
| | | MGC5576 | protein 106C | | GAATTGGTTCACTTAACTCCCAGCA (SEQ ID NO: 3725) |
| | | | | | AAACATCCTCCTGCCACTTAGGAGG (SEQ ID NO: 3726) |
| | | | | | AACACCTCCCTATGGTACCATTTAT (SEQ ID NO: 3727) |
| | | | | | AGCAGAATCAGTGCCTAGCCTGTGC (SEQ ID NO: 3728) |
| | | | | | TGAGTGACCTGCAGGCCATTATCAG (SEQ ID NO: 3729) |
| | | | | | GCCATTATCAGTGCCTCATCTGTGC (SEQ ID NO: 3730) |
| | | | | | GAGAGGGACCATCCAAATACCTAAG (SEQ ID NO: 3731) |
| | | | | | TTTCAGCTGTTCCCAAAGGCCTGGG (SEQ ID NO: 3732) |
| | | | | | ATGTGGGGCTTGATTCACCCTTCAT (SEQ ID NO: 3733) |
| | | | | | TTCACCCTTCATCCATTGGCTGGAA (SEQ ID NO: 3734) |
| 202475_at | TMEM147 | MGC1936, | transmembrane | 10430 | AAGGCGGCATCTATGACTTCATTGG (SEQ ID NO: 3735) |
| | | NIFIE14 | protein 147 | | CCTTGTCATGTCCCGGAATGCCGGC (SEQ ID NO: 3736) |
| | | | | | TGCTGAGCTTATTATGTCCCGCTGC (SEQ ID NO: 3737) |
| | | | | | AGCCCGGGGCATTGAGTTTGACTGG (SEQ ID NO: 3738) |
| | | | | | ATCAGTCTGGGTCCATACATCGTCG (SEQ ID NO: 3739) |
| | | | | | CGCGTCTGCTCAGGTCTGGATGATA (SEQ ID NO: 3740) |
| | | | | | TAACACGCTATGATCTGTACCACAC (SEQ ID NO: 3741) |
| | | | | | CTCCTGCTGATGTTCCTCAGAGTTT (SEQ ID NO: 3742) |
| | | | | | TATGGAGACGTTTGTCCACCTATGC (SEQ ID NO: 3743) |
| | | | | | TTGCCGTTGTCAATGTGCACTCCTA (SEQ ID NO: 3744) |
| | | | | | TCCTAGGCTTGGTGTCTCAGACATT (SEQ ID NO: 3745) |
| 218804_at | TMEM16A | FLJ10261, | transmembrane | 55107 | CACCAAGCCGACCTCAGAGTTGTTC (SEQ ID NO: 3746) |
| | | ORAOV2, | protein 16A | | AGAGTTGTTCATCTTCCTTATGGGA (SEQ ID NO: 3747) |
| | | TAOS2, | | | TATGGGACAAAACCGGTTGACCAGA (SEQ ID NO: 3748) |
| | | TMEM16A | | | GAGAGATGACCTCGGAAGCATTTCC (SEQ ID NO: 3749) |
| | | | | | GCATTTCCACAGATGGTGTCAGGGT (SEQ ID NO: 3750) |
| | | | | | TCCCCTGGAAGCTTTAGAATATTTA (SEQ ID NO: 3751) |
| | | | | | GGATTTTTTTTCTGTAGCTCAAAGG (SEQ ID NO: 3752) |
| | | | | | ATTTTGGTAACACTTCTCTATATTT (SEQ ID NO: 3753) |
| | | | | | GCAATGATACTAAACAACTCTCTGA (SEQ ID NO: 3754) |
| | | | | | AACTCTCTGAAATTTCTCAAGCACC (SEQ ID NO: 3755) |
| | | | | | AACATCATTTTAGCAAAGGCCAGGA (SEQ ID NO: 3756) |
| 218465_at | TMEM33 | 1600019D15Rik, | transmembrane | 55161 | GCATTTCAGTCTCAACGCATAGATA (SEQ ID NO: 3757) |
| | | FLJ10525, | protein 33 | | AAATCCTGTTCATTGAACTCCCATC (SEQ ID NO: 3758) |
| | | SHINC3 | | | GAACTCCCATCAACTCTTATGAAAT (SEQ ID NO: 3759) |
| | | | | | TCATGCTGATCTTCATTACCGTTGC (SEQ ID NO: 3760) |
| | | | | | TTGTGACCAGTTTCTGTCTGCATGT (SEQ ID NO: 3761) |
| | | | | | GTCTGCATGTAATTTGGATTTCTCA (SEQ ID NO: 3762) |
| | | | | | TATTTTTGTTCATCTCCTTATCTAT (SEQ ID NO: 3763) |
| | | | | | TTTGTAATATTTGTCCACTAAGCTG (SEQ ID NO: 3764) |
| | | | | | AAGCAGCCTCATACAGTTGATTTTG (SEQ ID NO: 3765) |
| | | | | | GTTGATTTTGTGTATGTGGCTAGTC (SEQ ID NO: 3766) |
| | | | | | GTGGCTAGTCTTATTGTCACTATGT (SEQ ID NO: 3767) |
| 212204_at | TMEM87A | DKFZp564G2022 | transmembrane | 25963 | GCTTAACTCTTTTGACATCTGCTAT (SEQ ID NO: 3768) |
| | | | protein 87A | | TATTGTGACACATCCCATTGCTGGC (SEQ ID NO: 3769) |
| | | | | | GGTGCACACTCCGAAACTTTTAACT (SEQ ID NO: 3770) |
| | | | | | ACTACTGTTTTGTAAGCCTCCAAGG (SEQ ID NO: 3771) |
| | | | | | GCCTCCAAGGGTGGCATTGCAGGGT (SEQ ID NO: 3772) |
| | | | | | GAGAGGTGCTCCAAGTGCTGTGATT (SEQ ID NO: 3773) |
| | | | | | GAGCACCGTGCTAGAGGAACTGTAA (SEQ ID NO: 3774) |
| | | | | | ATGAAGTAGCGTGGAGGCCCTGGAC (SEQ ID NO: 3775) |
| | | | | | ACTGCTGCTCGTTCTTTAGGATGGA (SEQ ID NO: 3776) |
| | | | | | GGACTGTTCTGGTATCTGGTATTGG (SEQ ID NO: 3777) |
| | | | | | GAAGCACTCTATTTCTGTTTTAATG (SEQ ID NO: 3778) |
| 218419_s_at | TMUB2 | FP2653, | transmembrane and | 79089 | TCTCAGCCAAATACTCATCTTTTGA (SEQ ID NO: 3779) |
| | | MGC3123 | ubiquitin-like domain | | CTGAAATCACACTGGCGGGAATGAA (SEQ ID NO: 3780) |
| | | | containing 2 | | GGCGGGAATGAAGATTGTGCCAGCC (SEQ ID NO: 3781) |
| | | | | | AGCAGGAATAGGGTGTCCTCCCTTC (SEQ ID NO: 3782) |
| | | | | | AAGCACTTTGCTTGCATTTTATTTT (SEQ ID NO: 3783) |
| | | | | | GAGTCCTTCATAGAGCTCAGTCAGG (SEQ ID NO: 3784) |
| | | | | | GGGATGGGGCACCAAGCCAAGCCCC (SEQ ID NO: 3785) |
| | | | | | TCCTCAGGCTGTAAGCAAGAGACAG (SEQ ID NO: 3786) |
| | | | | | CCTGCCCAACTCCAAGGACTGGGTA (SEQ ID NO: 3787) |
| | | | | | GGGCTATTGGAGGGTCAGTGTCTGT (SEQ ID NO: 3788) |
| | | | | | AGTGTCTGTGACTGAATAAAGTTCC (SEQ ID NO: 3789) |
| 209294_x_at | TNFRSF10B | CD262, DR5, | tumor necrosis factor | 8795 | GGTGATCCCACTGAGACTCTGAGAC (SEQ ID NO: 3790) |
| | | KILLER, | receptor superfamily, | | ATGACTTTGCAGACTTGGTGCCCTT (SEQ ID NO: 3791) |
| | | KILLER/DR5, | member 10b | | ACTCCTGGGAGCCGCTCATGAGGAA (SEQ ID NO: 3792) |
| | | TRAIL-R2, | | | GGACACCTTGTACACGATGCTGATA (SEQ ID NO: 3793) |
| | | TRAILR2, | | | CAAAACCGGGCGAGATGCCTCTGTC (SEQ ID NO: 3794) |
| | | TRICK2, | | | GAGGACCACTTGTTGAGCTCTGGAA (SEQ ID NO: 3795) |
| | | TRICK2A, | | | AATGCAGACTCTGCCATGTCCTAAG (SEQ ID NO: 3796) |
| | | TRICK2B, | | | CAGGAAGTCAGACCTTCCCTGGTTT (SEQ ID NO: 3797) |
| | | TRICKB, | | | GGAAAAAGCCCAACTGGACTCCAGT (SEQ ID NO: 3798) |
| | | ZTNFR9 | | | GGTACTGGAAGAAACTCTCCCATCC (SEQ ID NO: 3799) |
| | | | | | CATCCTGTAACTTTTCACTGCACTT (SEQ ID NO: 3800) |
| 207037_at | TNFRSF11A | CD265, FEO, | tumor necrosis factor | 8792 | GCCTGCTTTACGTATTTTCTTTTGT (SEQ ID NO: 3801) |
| | | LOH18CR1, | receptor superfamily, | | TGCCCCTGCTCACAGTGTTTTAGAG (SEQ ID NO: 3802) |
| | | ODFR, OFE, | member 11a, NFKB | | GTTTTAGAGATGGCTTTCCCAGTGT (SEQ ID NO: 3803) |
| | | OSTS, PDB2, | activator | | GTGAGGCCTGGAGATAGTTGCTAAG (SEQ ID NO: 3804) |
| | | RANK, | | | GAAACCCGATTTATTTCTCCTGAAT (SEQ ID NO: 3805) |
| | | TRANCER | | | AAGTTTGTGTCGTTCCTTAAGCAGA (SEQ ID NO: 3806) |
| | | | | | TGACCTTACCCGCTAGGTGGTTAAT (SEQ ID NO: 3807) |
| | | | | | GGTGGTTAATTTATCCATGCTGGCA (SEQ ID NO: 3808) |
| | | | | | GCAGAGGCACTCAGGTACTTGGTAA (SEQ ID NO: 3809) |
| | | | | | CAAGTTGCTGCAGCTTGGCATTCTT (SEQ ID NO: 3810) |
| | | | | | TTCTTCTTATTCTAGAGGTCTCTCT (SEQ ID NO: 3811) |
| 211841_s_at | TNFRSF25 | RP4-650H14.2, | tumor necrosis factor | 8718 | AGTGAGAAGATCTGCACCGTCCAGT (SEQ ID NO: 3812) |
| | | APO-3, DDR3, | receptor superfamily, | | TGCACCGTCCAGTTGGTGGGTAACA (SEQ ID NO: 3813) |
| | | DR3, LARD, | member 25 | | TGCCCGCAGGTGACATGGTCCTGGG (SEQ ID NO: 3814) |
| | | TNFRSF12, TR3, | | | ACATGGTCCTGGGACCAGTTGCCCA (SEQ ID NO: 3815) |
| | | TRAMP, WSL-1, | | | CGCTGGAAGGAGTTCGTGCGCACGC (SEQ ID NO: 3816) |
| | | WSL-LR | | | GCTGGGGCTGCGCGAGGCAGAGATC (SEQ ID NO: 3817) |
| | | | | | GCAGAGATCGAAGCCGTGGAGGTGG (SEQ ID NO: 3818) |
| | | | | | CTTCCGAGACCAGCAGTACGAGATG (SEQ ID NO: 3819) |
| | | | | | GCCCTGGAGCGCATGGGGCTGGACG (SEQ ID NO: 3820) |
| | | | | | GCTGGACGGCTGCGTGGAAGACTTG (SEQ ID NO: 3821) |
| | | | | | TGCGTGGAAGACTTGCGCAGCCGCC (SEQ ID NO: 3822) |
| 202687_s_at | TNFSF10 | APO2L, Apo-2L, | tumor necrosis factor | 8743 | GTAGCAGCTCACATAACTGGGACCA (SEQ ID NO: 3823) |
| | | CD253, TL2, | (ligand) superfamily, | | CATTGTCTTCTCCAAACTCCAAGAA (SEQ ID NO: 3824) |
| | | TRAIL | member 10 | | TGGGCCGCAAAATAAACTCCTGGGA (SEQ ID NO: 3825) |
| | | | | | GGGCATTCATTCCTGAGCAACTTGC (SEQ ID NO: 3826) |
| | | | | | TTTTACTACATCTATTCCCAAACAT (SEQ ID NO: 3827) |
| | | | | | TCCCAAACATACTTTCGATTTCAGG (SEQ ID NO: 3828) |
| | | | | | AATACACAAGTTATCCTGACCCTAT (SEQ ID NO: 3829) |
| | | | | | GGACTCTATTCCATCTATCAAGGGG (SEQ ID NO: 3830) |
| | | | | | TGAGCACTTGATAGACATGGACCAT (SEQ ID NO: 3831) |
| | | | | | GGACCATGAAGCCAGTTTTTTCGGG (SEQ ID NO: 3832) |
| | | | | | GCCTTTTTAGTTGGCTAACTGACCT (SEQ ID NO: 3833) |
| 211828_s_at | TNIK | | TRAF2 and NCK | 23043 | GAAATATATGCTTGGGCTCCTAAAC (SEQ ID NO: 3834) |
| | | | interacting kinase | | AAGTCTTTTGCAGATCTCCAGCACA (SEQ ID NO: 3835) |
| | | | | | GCACAAGCCTCTGCTAGTTGATCTC (SEQ ID NO: 3836) |
| | | | | | GGTTCACACACTGGTTTCCATGTAA (SEQ ID NO: 3837) |
| | | | | | CATACCATCTCATATTCAGGGCAAT (SEQ ID NO: 3838) |
| | | | | | TTCAGGGCAATATCACTCCTCATGC (SEQ ID NO: 3839) |
| | | | | | CCTCATGCTATTGTCATCTTGCCTA (SEQ ID NO: 3840) |
| | | | | | GTAAACACCTATGGCCGGATAACTA (SEQ ID NO: 3841) |
| | | | | | GTCTGTGGCCTACATTCATTCCAAT (SEQ ID NO: 3842) |
| | | | | | GGTATTTTTTGCATCCGTGCGATCT (SEQ ID NO: 3843) |
| | | | | | TAGCCAAGTGTTTTTCATGACCCTC (SEQ ID NO: 3844) |
| 213107_at | TNIK | | TRAF2 and NCK | 23043 | AATCAGCAAGATATCCTCCTCATGG (SEQ ID NO: 3845) |
| | | | interacting kinase | | GGTCCCTTTAGCTCTCAAAAGCAAT (SEQ ID NO: 3846) |
| | | | | | TCCTGTTCTCATTTTTACTGCTGTG (SEQ ID NO: 3847) |
| | | | | | CATTTTTACTGCTGTGGTTGTGCTG (SEQ ID NO: 3848) |
| | | | | | GCTGCTGAACAATACTATCTTCTCA (SEQ ID NO: 3849) |
| | | | | | AATTCCATGCCACAAATTCAGCAAT (SEQ ID NO: 3850) |
| | | | | | ACTGTAATTGGATGCTGATGTGGAC (SEQ ID NO: 3851) |
| | | | | | CACTCCCGAATGTGATTGCCACCAG (SEQ ID NO: 3852) |
| | | | | | GATTGCCACCAGCTCTTTATATTGC (SEQ ID NO: 3853) |
| | | | | | GCTCTTTATATTGCTGCTGTGGTAT (SEQ ID NO: 3854) |
| | | | | | TGCAGTCTTGTTTACAGTATGTACA (SEQ ID NO: 3855) |
| 213109_at | TNIK | | TRAF2 and NCK | 23043 | AACTCATGTGGACTTATGCCAGTCT (SEQ ID NO: 3856) |
| | | | interacting kinase | | TTGGTTGAACATATCGCTTTCCCTT (SEQ ID NO: 3857) |
| | | | | | TCCCAGTACAGTCCATCTTTCAATG (SEQ ID NO: 3858) |
| | | | | | ATCTTTCAATGTTGCAGCCTGGTTG (SEQ ID NO: 3859) |
| | | | | | GGCAGGAATTTCCAGGAGATCCCCA (SEQ ID NO: 3860) |
| | | | | | TCCCCAAGAATGCTGCCTTGTCTGT (SEQ ID NO: 3861) |
| | | | | | CAAAGATGGACCATGTGCCCTTCGG (SEQ ID NO: 3862) |
| | | | | | TTTACCCCTTTACTCTGCAAGAATG (SEQ ID NO: 3863) |
| | | | | | AAATTCTGTCTGCCTACTAGTTTTA (SEQ ID NO: 3864) |
| | | | | | CAATAAATGTACCTTATCTCCTTAG (SEQ ID NO: 3865) |
| | | | | | GGCTGAAGGCCATAACTACATAGTG (SEQ ID NO: 3866) |
| 220339_s_at | TPSG1 | PRSS31, TMT, | tryptase gamma 1 | 25823 | GGTGAAAGTCTCCGTGGTGGACACA (SEQ ID NO: 3867) |
| | | trpA | | | GCCTCTGGTCTGCCAGGTGAACGGT (SEQ ID NO: 3868) |
| | | | | | AGGTGAACGGTGCCTGGGTGCAGGC (SEQ ID NO: 3869) |
| | | | | | GGTGCAGGCTGGCATTGTGAGCTGG (SEQ ID NO: 3870) |
| | | | | | CCGCCACATCACAGCATCAGGGGGC (SEQ ID NO: 3871) |
| | | | | | CTCAGAGTCTGGGTACCCCAGGCTC (SEQ ID NO: 3872) |
| | | | | | GCTGCACCCATCTGCGGATGGTACT (SEQ ID NO: 3873) |
| | | | | | CCGCCCCTGACTGATGGCAGGAATC (SEQ ID NO: 3874) |
| | | | | | ATAAGTTACTATTTATTCCGCTCCG (SEQ ID NO: 3875) |
| | | | | | TCCCTCTCCCTTGAGAAGCTGAGTC (SEQ ID NO: 3876) |
| | | | | | GAAGCTGAGTCTTCTGCATCAGATT (SEQ ID NO: 3877) |
| 213593_s_at | TRA2A | HSU53209 | transformer-2 alpha | 29896 | GGTGGGACGAGCCTAATGAAGTGGT (SEQ ID NO: 3878) |
| | | | | | GAAGTGGTTAGTCAACGACCGCACC (SEQ ID NO: 3879) |
| | | | | | GCACCGACCCTTAAGCGTTCTTAAG (SEQ ID NO: 3880) |
| | | | | | AAGCGTTCTTAAGACGAGAGCTGTG (SEQ ID NO: 3881) |
| | | | | | GTGAGGGGAAACTACGCAAGGAGCC (SEQ ID NO: 3882) |
| | | | | | GAATGTGGAAATGCTGTCACGCCTT (SEQ ID NO: 3883) |
| | | | | | TCACGCCTTATTTTTCTAATTCCTC (SEQ ID NO: 3884) |
| | | | | | TCTAATTCCTCGATGTTGGGTTTTT (SEQ ID NO: 3885) |
| | | | | | CTCTTCTTTATATCTCAACTTTTAA (SEQ ID NO: 3886) |
| | | | | | TCTCAACTTTTAATGCTCTTCTTCC (SEQ ID NO: 3887) |
| | | | | | GAACGAATTCATTGGCTCAGTATTT (SEQ ID NO: 3888) |
| 219736_at | TRIM36 | HAPRIN, | tripartite motif- | 55521 | CATTCTATCTAAATTGTGTGTCTGT (SEQ ID NO: 3889) |
| | | RBCC728, | containing 36 | | GTGTGTCTGTTTTTGAAAGGTGTGT (SEQ ID NO: 3890) |
| | | RNF98 | | | AGGTGTGTGCTTTCAAGTAGGTGGT (SEQ ID NO: 3891) |
| | | | | | TATGCATTGTAGCAGTAATCATTTA (SEQ ID NO: 3892) |
| | | | | | GGAATGCTTTTGACATGTTTTAACA (SEQ ID NO: 3893) |
| | | | | | TTAATGGAAATCTGTTACTCAGGCT (SEQ ID NO: 3894) |
| | | | | | TTAGCTTTTTACCCTCAACTCAAGT (SEQ ID NO: 3895) |
| | | | | | AGCAGGCTGTTAATTTCCCATCTAT (SEQ ID NO: 3896) |
| | | | | | ATTTCCCATCTATGAATAGCACTTT (SEQ ID NO: 3897) |
| | | | | | ATGCAGCGTTATGTGGATCATTATG (SEQ ID NO: 3898) |
| | | | | | TTGTGGTTGATACTCAAGCCTTGTA (SEQ ID NO: 3899) |
| 209890_at | TSPAN5 | Tm4sf9, Tspan-5 | tetraspanin 5 | 10098 | CACAGATTCCAATGCAAGTCGAGAG (SEQ ID NO: 3900) |
| | | | | | AGTCGAGAGCGATGTGGCGTTCCAT (SEQ ID NO: 3901) |
| | | | | | CCTGCTGCACTAAAGATCCCGCAGA (SEQ ID NO: 3902) |
| | | | | | CGAAAGGCTGTGTGCCCCAGTTTGA (SEQ ID NO: 3903) |
| | | | | | GCAGGACAATTTAACCATCGTTGCT (SEQ ID NO: 3904) |
| | | | | | CAGATATTTGGGATATGCCTGGCCC (SEQ ID NO: 3905) |
| | | | | | GCCTGGCCCAGAATTTGGTTAGCGA (SEQ ID NO: 3906) |
| | | | | | ATCGAAGCTGTCAGGGCGAGCTGGT (SEQ ID NO: 3907) |
| | | | | | GCTGCTGCAAGACACTGGACAGACC (SEQ ID NO: 3908) |
| | | | | | CTTCGAGCTGCATGGACCTAATCAC (SEQ ID NO: 3909) |
| | | | | | CACAGATGCAGCCTGCAGTCTCGCC (SEQ ID NO: 3910) |
| 213726_x_at | TUBB2C | RP13-122B23.2, | tubulin, beta 2C | 10383 | CCACCTTCATTGGCAACAGCACGGC (SEQ ID NO: 3911) |
| | | TUBB2 | | | ACAGCACGGCCATCCAGGAGCTGTT (SEQ ID NO: 3912) |
| | | | | | CAGGAGCTGTTCAAGCGCATCTCCG (SEQ ID NO: 3913) |
| | | | | | TGCACTGGTACACGGGCGAGGGCAT (SEQ ID NO: 3914) |
| | | | | | ACGAGATGGAGTTCACCGAGGCCGA (SEQ ID NO: 3915) |
| | | | | | GTACCAGCAGTACCAGGATGCCACA (SEQ ID NO: 3916) |
| | | | | | GGGCGAGTTCGAGGAGGAGGCTGAG (SEQ ID NO: 3917) |
| | | | | | GAGGAGGAGGTGGCCTAGAGCCTTC (SEQ ID NO: 3918) |
| | | | | | TGTGAACTCTTTATTCACTCCCAGC (SEQ ID NO: 3919) |
| | | | | | GTCCACATCCATGCTGTACAGACAC (SEQ ID NO: 3920) |
| | | | | | GTACAGACACCACCATTAAAGCATT (SEQ ID NO: 3921) |
| 218382_s_at | U2AF2 | U2AF65 | U2 small nuclear | 11338 | GCAAGTGCCGGGCTTGATGAGCTCC (SEQ ID NO: 3922) |
| | | | RNA auxiliary factor 2 | | AGCTCCCAGGTGCAGATGGGCGGCC (SEQ ID NO: 3923) |
| | | | | | GAGGTCCTGTGCCTCATGAACATGG (SEQ ID NO: 3924) |
| | | | | | GAGGATGTGCGGGACGAGTGCAGCA (SEQ ID NO: 3925) |
| | | | | | GTACGGGCTTGTCAAGTCCATCGAG (SEQ ID NO: 3926) |
| | | | | | AGGTGCCCGGCTGCGGAAAGATCTT (SEQ ID NO: 3927) |
| | | | | | GGAGTTCACCTCTGTGTTTGACTGC (SEQ ID NO: 3928) |
| | | | | | GGCCGCAAGTTCGCCAACAGAGTGG (SEQ ID NO: 3929) |
| | | | | | CACAAAATACTGTGACCCCGACTCT (SEQ ID NO: 3930) |
| | | | | | CCGGGACTTCTGGTAGAGGCGGCTG (SEQ ID NO: 3931) |
| | | | | | GCAGAGGAGTGACAGCCGCAGACAC (SEQ ID NO: 3932) |
| 215057_at | U66046* | | Clone 161455-2-3 B | | ACCAATGTTCAGCCTCTGAGACCAG (SEQ ID NO: 3933) |
| | | | cell expressed mRNA | | AAGCAGTGGCCATGAATGTTCCCTC (SEQ ID NO: 3934) |
| | | | from chromosome X | | GAAGAAGTGTACTCACACTGCCCCA (SEQ ID NO: 3935) |
| | | | | | ACTGCCCCAACACCAGTGAGTGGAA (SEQ ID NO: 3936) |
| | | | | | TATTTATAGGTCTCTCATTTGGTTT (SEQ ID NO: 3937) |
| | | | | | CACAAAATTAACCATTACCCACATT (SEQ ID NO: 3938) |
| | | | | | CATTCCTACCAACAAGAAGTAACCA (SEQ ID NO: 3939) |
| | | | | | TGCAGACAACATATTAGCACATTGC (SEQ ID NO: 3940) |
| | | | | | TCCATGTTCTTCCCATTTATACAGG (SEQ ID NO: 3941) |
| | | | | | CATTTATACAGGAGCAATTCTTGCA (SEQ ID NO: 3942) |
| | | | | | TCCTTGCTCCTCAAATGCCTTAGAG (SEQ ID NO: 3943) |
| 201377_at | UBAP2L | FLJ42300, | ubiquitin associated | 9898 | CCCCCTTACAAGCATTTCTGGACGG (SEQ ID NO: 3944) |
| | | KIAA0144, NICE-4 | protein 2-like | | GCTGAGAGCTAATTTGGCCCAAGGC (SEQ ID NO: 3945) |
| | | | | | ACCAGACCACTGAGGAGAGCCTGCT (SEQ ID NO: 3946) |
| | | | | | CATGGCCTGCGTGGCTTGGGGAAAT (SEQ ID NO: 3947) |
| | | | | | GAGTTGGTGGATACCTTCTGGGCTT (SEQ ID NO: 3948) |
| | | | | | TTCTGGGCTTTTGAACTTGCCCCTC (SEQ ID NO: 3949) |
| | | | | | CTGTCTTACCCATTTCAAGTTCAAG (SEQ ID NO: 3950) |
| | | | | | TTCAAGTTCAAGCGGTGCAGCACCT (SEQ ID NO: 3951) |
| | | | | | GTGCAGCACCTTCGAAGCATCAATG (SEQ ID NO: 3952) |
| | | | | | AAGCATCAATGCACACACCTGCTGT (SEQ ID NO: 3953) |
| | | | | | GCTGTTGCTTTTGATTTCTGGAAGG (SEQ ID NO: 3954) |
| 200683_s_at | UBE2L3 | E2-F1, L-UBC, | ubiquitin-conjugating | 7332 | GAAGCCCTATAATCACAGGTCTGTG (SEQ ID NO: 3955) |
| | | UBCH7, UbcM4 | enzyme E2L 3 | | ATGAATCTCGCCAGAAAGGCTCCTG (SEQ ID NO: 3956) |
| | | | | | TTCTCCCTGCAGCCATTGTAGAAGA (SEQ ID NO: 3957) |
| | | | | | TGTAGAAGATCTGCTGGTCCTTGCA (SEQ ID NO: 3958) |
| | | | | | AAGCTACAGCCAGAATGTCCGTTTG (SEQ ID NO: 3959) |
| | | | | | TGTCCGTTTGAAACTCCTAGCTCAT (SEQ ID NO: 3960) |
| | | | | | CTAGCTCATCTGTCACCGAGCTTCA (SEQ ID NO: 3961) |
| | | | | | CGAGCTTCATCCGAATGTGCCACGG (SEQ ID NO: 3962) |
| | | | | | GCACACTTTGACCCTTTGTAGGATT (SEQ ID NO: 3963) |
| | | | | | TAAAGCACCAGTCTGGGGTCGCCTG (SEQ ID NO: 3964) |
| | | | | | AGCTCACTCAGTAATATCCTTTCAA (SEQ ID NO: 3965) |
| 201649_at | UBE2L6 | MGC40331, RIG- | ubiquitin-conjugating | 9246 | GTGTGTTGTGTATGGACTCACTCCC (SEQ ID NO: 3966) |
| | | B, UBCH8 | enzyme E2L 6 | | CCTTTACATTCCCCAGAGCCAAGGG (SEQ ID NO: 3967) |
| | | | | | GTTTGCAGTTACAGGCCAGTTCTCC (SEQ ID NO: 3968) |
| | | | | | TTCTCCAGCTCTCCATCTTAGAGAG (SEQ ID NO: 3969) |
| | | | | | TGAATCCAGCAGCCAACTCGAATCC (SEQ ID NO: 3970) |
| | | | | | CCCCCTAGGGCTCAGGCACTGAGGG (SEQ ID NO: 3971) |
| | | | | | GGAGGGTACCCTATTTACAACTGAG (SEQ ID NO: 3972) |
| | | | | | GAGTCAGCCAAGCCACTGATGGGAA (SEQ ID NO: 3973) |
| | | | | | GTATATATCCTCCAGCATTCAGTCC (SEQ ID NO: 3974) |
| | | | | | CCAGGGGGAGCCACGGAAACCATGT (SEQ ID NO: 3975) |
| | | | | | GAAACCATGTTCTTGCTTAAGCCAT (SEQ ID NO: 3976) |
| 208997_s_at | UCP2 | SLC25A8, UCPH | uncoupling protein 2 | 7351 | TAAAGGTCCGATTCCAAGCTCAGGC (SEQ ID NO: 3977) |
| | | | (mitochondrial, proton | | ACCAAAGCACCGTCAATGCCTACAA (SEQ ID NO: 3978) |
| | | | carrier) | | CTCTCCCAATGTTGCTCGTAATGCC (SEQ ID NO: 3979) |
| | | | | | TGAGCTGGTGACCTATGACCTCATC (SEQ ID NO: 3980) |
| | | | | | GCCAACCTCATGACAGATGACCTCC (SEQ ID NO: 3981) |
| | | | | | CCTCCCCTGTAGACGTGGTCAAGAC (SEQ ID NO: 3982) |
| | | | | | AGACGAGATACATGAACTCTGCCCT (SEQ ID NO: 3983) |
| | | | | | CTGCCCTGGGCCAGTACAGTAGCGC (SEQ ID NO: 3984) |
| | | | | | TACAAAGGGTTCATGCCCTCCTTTC (SEQ ID NO: 3985) |
| | | | | | AGCTGAAACGAGCCCTCATGGCTGC (SEQ ID NO: 3986) |
| | | | | | GAGAGGCTCCCTTCTGAGCCTCTCC (SEQ ID NO: 3987) |
| 208998_at | UCP2 | SLC25A8, UCPH | uncoupling protein 2 | 7351 | TCTACATTCTCATCTACTCATTGTC (SEQ ID NO: 3988) |
| | | | (mitochondrial, proton | | ACATTCTCATCTACTCATTGTCTCA (SEQ ID NO: 3989) |
| | | | carrier) | | CTCATTGTCTCAGTGCTGGTGGAGT (SEQ ID NO: 3990) |
| | | | | | GTCTCAGTGCTGGTGGAGTTGACAT (SEQ ID NO: 3991) |
| | | | | | AGTGCTGGTGGAGTTGACATTTGAC (SEQ ID NO: 3992) |
| | | | | | CCGTCCCTTGGAAAGTTCAGCCAGA (SEQ ID NO: 3993) |
| | | | | | GAAAGTTCAGCCAGAATCTTCGTCC (SEQ ID NO: 3994) |
| | | | | | AAGTTCAGCCAGAATCTTCGTCCTG (SEQ ID NO: 3995) |
| | | | | | TAGCCCACTTGTCATCCATAAAGCA (SEQ ID NO: 3996) |
| | | | | | AGCCCACTTGTCATCCATAAAGCAA (SEQ ID NO: 3997) |
| | | | | | GTCATCCATAAAGCAAGCTCAACCT (SEQ ID NO: 3998) |
| 212074_at | UNC84A | FLJ12407, | unc-84 homolog A | 23353 | AATGAGATATCTACAAGGCACTTAA (SEQ ID NO: 3999) |
| | | KIAA0810, | (C. elegans) | | GTGTTACAGATGTTTTACCTTAAGA (SEQ ID NO: 4000) |
| | | MGC176649, | | | GACAGTTTTCAGTGTACCGTAAATG (SEQ ID NO: 4001) |
| | | SUN1 | | | GTACCGTAAATGTTGTGTTTTCAGA (SEQ ID NO: 4002) |
| | | | | | GATGGTGCTGACTGGTTTTCTGTAT (SEQ ID NO: 4003) |
| | | | | | ATTGCACAACAGTCCTCAAATACAC (SEQ ID NO: 4004) |
| | | | | | AACTATTCATACATCAAGCAGCATT (SEQ ID NO: 4005) |
| | | | | | TTTTCACTCTCCTTAGAATTGGAAC (SEQ ID NO: 4006) |
| | | | | | AATGCTTTTCAAAATATCTGTCTTT (SEQ ID NO: 4007) |
| | | | | | ATCTGTCTTTGGTAGTGCTTCTGCT (SEQ ID NO: 4008) |
| | | | | | CTGCTGCCACCAAATTGATAAGATG (SEQ ID NO: 4009) |
| 208909_at | UQCRFS1 | RIP1, RIS1, | ubiquinol-cytochrome | 7386 | GAGAGGCAAACCCCTGTTTGTGCGT (SEQ ID NO: 4010) |
| | | RISP, UQCR5 | c reductase, Rieske | | CTGTTTGTGCGTCATAGAACCCAGA (SEQ ID NO: 4011) |
| | | | iron-sulfur | | GGACCCACAGCATGATCTAGATCGA (SEQ ID NO: 4012) |
| | | | polypeptide 1 | | GATAGGTGTTTGCACTCATCTTGGC (SEQ ID NO: 4013) |
| | | | | | TCATCTTGGCTGTGTACCCATTGCA (SEQ ID NO: 4014) |
| | | | | | TTTTGGTGGTTATTACTGCCCTTGC (SEQ ID NO: 4015) |
| | | | | | GGGTCACACTATGATGCATCTGGCA (SEQ ID NO: 4016) |
| | | | | | CAGGATCAGATTGGGTCCTGCTCCT (SEQ ID NO: 4017) |
| | | | | | TGAAGTCCCCACGTATGAGTTCACC (SEQ ID NO: 4018) |
| | | | | | GGACTCAAGTCATAGGCTTCTTTCA (SEQ ID NO: 4019) |
| | | | | | GGCTTCTTTCAGTCTTTATGTCACC (SEQ ID NO: 4020) |
| 202233_s_at | UQCRH | MGC111572, | ubiquinol-cytochrome | 7388 | GCTCGTGTTGAATCTAGAACCGTAG (SEQ ID NO: 4021) |
| | | QCR6 | c reductase hinge | | AGAACCGTAGCCAGACATGGGACTG (SEQ ID NO: 4022) |
| | | | protein | | GACATGGGACTGGAGGACGAGCAAA (SEQ ID NO: 4023) |
| | | | | | GATGCTTACCGAATCCGGAGATCCT (SEQ ID NO: 4024) |
| | | | | | GATCCCCTAACAACAGTGAGAGAGC (SEQ ID NO: 4025) |
| | | | | | GGATTGCACGGAGGAGCTCTTTGAC (SEQ ID NO: 4026) |
| | | | | | AGCTCTTTGACTTCTTGCATGCGAG (SEQ ID NO: 4027) |
| | | | | | TGCATGCGAGGGACCATTGCGTGGC (SEQ ID NO: 4028) |
| | | | | | CATTGCGTGGCCCACAAACTCTTTA (SEQ ID NO: 4029) |
| | | | | | TGGCCCACAAACTCTTTAACAACTT (SEQ ID NO: 4030) |
| | | | | | GTAATTCGCAATGATTCCATCTAAA (SEQ ID NO: 4031) |
| 201568_at | UQCRQ | QCR8, QP-C, | ubiquinol-cytochrome | 27089 | TTGGTTCAGTTTTCCGGAGGGCGAG (SEQ ID NO: 4032) |
| | | | c reductase, complex | | TGGGCCGCGAGTTTGGGAATCTGAC (SEQ ID NO: 4033) |
| | | QPC | III subunit VII, 9.5 kDa | | GAATCTGACGCGGATGCGGCATGTG (SEQ ID NO: 4034) |
| | | | | | GTGATCAGCTACAGCTTGTCACCGT (SEQ ID NO: 4035) |
| | | | | | CTATCCGCACGTCTTCACTAAAGGA (SEQ ID NO: 4036) |
| | | | | | TAAAGGAATCCCCAATGTTCTGCGC (SEQ ID NO: 4037) |
| | | | | | CGCATTCGGGAGTCTTTCTTTCGCG (SEQ ID NO: 4038) |
| | | | | | TGGTGCCGCAGTTTGTAGTGTTTTA (SEQ ID NO: 4039) |
| | | | | | AGAGGAGGATCCAGCTGCCTATGAA (SEQ ID NO: 4040) |
| | | | | | AAATGAGCAACGCATCCGGATGACG (SEQ ID NO: 4041) |
| | | | | | TGTCTCTGAAAGACCTTTCTCTGGA (SEQ ID NO: 4042) |
| 213327_s_at | USP12 | USP12L1 | ubiquitin specific | 219333 | GACAGGAACCAGGAGGCTTGGGAAC (SEQ ID NO: 4043) |
| | | | peptidase 12 | | TATTATGTGGGTCTTGGGGCCTCTA (SEQ ID NO: 4044) |
| | | | | | GGGGCCTCTAACCATCAGTATAGGG (SEQ ID NO: 4045) |
| | | | | | TTTTTCTTTCCTTGATGGCAGTAGA (SEQ ID NO: 4046) |
| | | | | | GCAGTAGAAAGACCTCATTTTCATA (SEQ ID NO: 4047) |
| | | | | | AGACCTCATTTTCATAACATAACTA (SEQ ID NO: 4048) |
| | | | | | ACATAACTACTCTTGATACTTTCTT (SEQ ID NO: 4049) |
| | | | | | GATTCTATCATGAGGTATTTGGCTG (SEQ ID NO: 4050) |
| | | | | | TGAGGTATTTGGCTGGGAGCTGGGA (SEQ ID NO: 4051) |
| | | | | | CTTCAGTCTCCCTGTCTTGTAAAAT (SEQ ID NO: 4052) |
| | | | | | GGTTCTTGAAGAAGGCACTATATAA (SEQ ID NO: 4053) |
| 221654_s_at | USP3 | MGC129878, | ubiquitin specific | 9960 | TCTCACTTTGAGGCACATTTACATC (SEQ ID NO: 4054) |
| | | MGC129879, | peptidase 3 | | TACATCAATGCTTTTGTTCCTCTCA (SEQ ID NO: 4055) |
| | | SIH003, UBP | | | AAGCAAGATGTGTTCCTTATTGTGA (SEQ ID NO: 4056) |
| | | | | | GTGAAGAGCGACACAACTGCCTGCT (SEQ ID NO: 4057) |
| | | | | | GCTGCCTTTCCACAGCTATAATGGA (SEQ ID NO: 4058) |
| | | | | | TGGACATCAGGTTGACTCTAAATCA (SEQ ID NO: 4059) |
| | | | | | GTGTGCACAATACTTGTGGCCCACA (SEQ ID NO: 4060) |
| | | | | | GACCAGGTAATTACTGCTTGTCTCT (SEQ ID NO: 4061) |
| | | | | | TTACTGCTTGTCTCTCAAGGCTGCT (SEQ ID NO: 4062) |
| | | | | | TCAAGGCTGCTGTCTTTATCAGCAC (SEQ ID NO: 4063) |
| | | | | | GGTTCAGTTGTACTTGTCCTGCAAA (SEQ ID NO: 4064) |
| 205139_s_at | UST | 2OST | uronyl-2- | 10090 | AAACATTCATCCTCTTACTGCAAGT (SEQ ID NO: 4065) |
| | | | sulfotransferase | | TATCTGGGTACTTTTACCTGTGTGT (SEQ ID NO: 4066) |
| | | | | | AAGGCATTTCTTTTCAGCAGTGATC (SEQ ID NO: 4067) |
| | | | | | TGACGGATTTACTTACAGGGCCTTA (SEQ ID NO: 4068) |
| | | | | | GCCTTAATGTTATTTTGTCCCAGCC (SEQ ID NO: 4069) |
| | | | | | CACCCTCTAGGTCCTAAAAGTCAAG (SEQ ID NO: 4070) |
| | | | | | CCTGGGCCCAACAGTTTGTACTTCA (SEQ ID NO: 4071) |
| | | | | | ATTATAACCTTTCAGCACATTCCAA (SEQ ID NO: 4072) |
| | | | | | CATTCCAAGGTTTTAGTTACTCAGG (SEQ ID NO: 4073) |
| | | | | | ATTATTATGAACCTTCAGCCTACTT (SEQ ID NO: 4074) |
| | | | | | AGCCTACTTTCTTGAGTGCCGTAAA (SEQ ID NO: 4075) |
| 208626_s_at | VAT1 | FLJ20230, VATI | vesicle amine | 10493 | CAAAAACAGGTGCCCTCATCGTGGT (SEQ ID NO: 4076) |
| | | | transport protein 1 | | GTCCCGTCTCTATGGTGGTTGAGGA (SEQ ID NO: 4077) |
| | | | homolog (T. californica) | | AAAAATCCAAGCACATTCCCCTTGC (SEQ ID NO: 4078) |
| | | | | | GCCCTACTGCCTTCTCAAAGCAGAG (SEQ ID NO: 4079) |
| | | | | | GGATGGTCAGGACCTGGGCCATTGC (SEQ ID NO: 4080) |
| | | | | | GGTAGGGAGGTCACTCCCTCTACTC (SEQ ID NO: 4081) |
| | | | | | CTCCCTCTACTCACTGAGCTAGGAT (SEQ ID NO: 4082) |
| | | | | | TAGGGAGGGTTATTGCCCCAACCAT (SEQ ID NO: 4083) |
| | | | | | GAGGTGGAGGGACAGGCTCAGCCTC (SEQ ID NO: 4084) |
| | | | | | GTGAAGTGCGATTTCTGCTTTTGTG (SEQ ID NO: 4085) |
| | | | | | CCATTACCACAGCTGCCTTTGTGTG (SEQ ID NO: 4086) |
| 210849_s_at | VPS41 | HVPS41, | vacuolar protein | 27072 | ATTTCTTCATCTATTCCTGTACTAA (SEQ ID NO: 4087) |
| | | HVSP41, | sorting 41 homolog | | AGAATTTGGGGCTCTCTTCATGCCT (SEQ ID NO: 4088) |
| | | hVps41p | (S. cerevisiae) | | CAACTGAGAATTCTCCCAGCTGCCT (SEQ ID NO: 4089) |
| | | | | | GCCTGAAAGCGTCGCCAACTGTGGT (SEQ ID NO: 4090) |
| | | | | | GCAAGCTGCTACCTGCAACTTGGAC (SEQ ID NO: 4091) |
| | | | | | AACTTGGACGTTGTTTCCACGTGCT (SEQ ID NO: 4092) |
| | | | | | CTGGCTACGATTCTTGCATTCTGGG (SEQ ID NO: 4093) |
| | | | | | GGCTTTTTTCTGTGTCATCAACTAT (SEQ ID NO: 4094) |
| | | | | | AGCAGGCTGATGTCCTGCACCATTA (SEQ ID NO: 4095) |
| | | | | | TTCTGTGTGCTGTGACGACTGTCAA (SEQ ID NO: 4096) |
| | | | | | AGTATCTGGCCATGGCGGACACTCA (SEQ ID NO: 4097) |
| 205126_at | VRK2 | | vaccinia related | 7444 | GCGCTGAGTCCTGTGCAACATGGAA (SEQ ID NO: 4098) |
| | | | kinase 2 | | AGCTATACACAATTCCCAAACTCAT (SEQ ID NO: 4099) |
| | | | | | GATTTTACCAGTCCAGATATATTCA (SEQ ID NO: 4100) |
| | | | | | ACACTTCCACAGTCAGCACGGGGAT (SEQ ID NO: 4101) |
| | | | | | TCAGCACGGGGATCACAGACTTAGA (SEQ ID NO: 4102) |
| | | | | | TGGACTTTGGCCTACAATTTCCCAG (SEQ ID NO: 4103) |
| | | | | | ATTTCCCAGTTTACTCTTAGTGAAG (SEQ ID NO: 4104) |
| | | | | | TATTATCGCATCATCATACCTGTCC (SEQ ID NO: 4105) |
| | | | | | CATACCTGTCCTTTTGATGTTAGTA (SEQ ID NO: 4106) |
| | | | | | TTCCAGCTCTTCACCGAAATGTTGT (SEQ ID NO: 4107) |
| | | | | | CTTATTTCAGTGTTTCCTTCCAGAC (SEQ ID NO: 4108) |
| 200628_s_at | WARS | GAMMA-2, IFI53, | tryptophanyl-tRNA | 7453 | GGACGTGTCTTTCATGTACCTGACC (SEQ ID NO: 4109) |
| | | IFP53 | synthetase | | GGACGACGACAAGCTCGAGCAGATC (SEQ ID NO: 4110) |
| | | | | | GAAGGATTACACCAGCGGAGCCATG (SEQ ID NO: 4111) |
| | | | | | GAGCCATGCTCACCGGTGAGCTCAA (SEQ ID NO: 4112) |
| | | | | | CATAGAGGTTCTGCAGCCCTTGATC (SEQ ID NO: 4113) |
| | | | | | GTCCTTCGACTTTCAGTAGCACTCG (SEQ ID NO: 4114) |
| | | | | | TCAATAATCCCAGCCCAGTCAAAGC (SEQ ID NO: 4115) |
| | | | | | ACCTGTAGGCTTCTGTCTCATGGTA (SEQ ID NO: 4116) |
| | | | | | GTTTCTTCCTGTGAGTTCCATTATT (SEQ ID NO: 4117) |
| | | | | | CAAAGCAGCCCCACAGTGGAGCCTT (SEQ ID NO: 4118) |
| | | | | | GTGGAGCCTTCGGCTGGAAGTCCAT (SEQ ID NO: 4119) |
| 200609_s_at | WDR1 | AIP1, NORI-1 | WD repeat domain 1 | 9948 | GGCTCTCACTATCTGTCAGGGGAGG (SEQ ID NO: 4120) |
| | | | | | GCAAAGCCGGAATTCTGTGTTGAAC (SEQ ID NO: 4121) |
| | | | | | TGAACAGATGTCTGTCTCCCTAGTG (SEQ ID NO: 4122) |
| | | | | | AGTGTGTGACTCACACCTTGTGGCT (SEQ ID NO: 4123) |
| | | | | | CGCCACCTCCAGATCAGATGGGGAC (SEQ ID NO: 4124) |
| | | | | | GACACACAACCCCTGGATATGTTTC (SEQ ID NO: 4125) |
| | | | | | GTTGCCTGTCAGTGTTTACAAACTA (SEQ ID NO: 4126) |
| | | | | | TGACGGCACCGTGTCCAAGTTTTTA (SEQ ID NO: 4127) |
| | | | | | TTTAGAACCCTTGTTAGCCAGACCG (SEQ ID NO: 4128) |
| | | | | | TAGCCAGACCGAGGTGTCCTGGTCA (SEQ ID NO: 4129) |
| | | | | | ACCATCATGCTTTGATGTTCCCCTG (SEQ ID NO: 4130) |
| 209592_s_at | WDR68 | AN11; HAN11 | WD repeat domain | 10238 | GATGGCTCGGTGCGGATGTTTGACC (SEQ ID NO: 4131) |
| | | | 68 | | ACAGCACCATCATTTACGAAGACCC (SEQ ID NO: 4132) |
| | | | | | CATTTACGAAGACCCACAGCATCAC (SEQ ID NO: 4133) |
| | | | | | TGCTTCGCCTCTGCTGGAACAAGCA (SEQ ID NO: 4134) |
| | | | | | GAGATCAACAATGTGCAGTGGGCAT (SEQ ID NO: 4135) |
| | | | | | CAGTGGGCATCAACTCAGCCCGACT (SEQ ID NO: 4136) |
| | | | | | CCATCTGCTACAACAACTGCCTGGA (SEQ ID NO: 4137) |
| | | | | | CTGCCTGGAGATACTCAGAGTGTAG (SEQ ID NO: 4138) |
| | | | | | AGAGTGTAGTGTTGGTGGCGCTGTG (SEQ ID NO: 4139) |
| | | | | | TGTGCCCACGAGGCAGGGGCTTTTG (SEQ ID NO: 4140) |
| | | | | | GCAGGGGCTTTTGTATTTCCTGCCT (SEQ ID NO: 4141) |
| 208459_s_at | XPO7 | KIAA0745, | exportin 7 | 23039 | TTATTGCAAGCCTGGAACCTCACGT (SEQ ID NO: 4142) |
| | | RANBP16 | | | GAACCTCACGTCATCATGTATATTC (SEQ ID NO: 4143) |
| | | | | | ACTTACTGCACTTGACACCATGGTA (SEQ ID NO: 4144) |
| | | | | | CCATGGTATGCACAGGCTGCTGCTC (SEQ ID NO: 4145) |
| | | | | | GACCACATTGTGACATACCTCTTCA (SEQ ID NO: 4146) |
| | | | | | TTCAAGCAGCTGTCACGTAGCACCA (SEQ ID NO: 4147) |
| | | | | | ACCACACCCCTGAACCAGGAGAGCG (SEQ ID NO: 4148) |
| | | | | | GCTGTCCACGGTGCTGAACATCATC (SEQ ID NO: 4149) |
| | | | | | TCCCGACCACTACTTGGCTTGATAT (SEQ ID NO: 4150) |
| | | | | | GCCAGCCACCGGAGAAGCAGCAGGC (SEQ ID NO: 4151) |
| | | | | | CCTGTCAGCATTCCGTCGAGAAGTC (SEQ ID NO: 4152) |
| 212166_at | XPO7 | KIAA0745, | exportin 7 | 23039 | ATTCCTGTTACTGTATTAACCCTTG (SEQ ID NO: 4153) |
| | | RANBP16 | | | GGAACTCTAAGCCATGCCAGAACAC (SEQ ID NO: 4154) |
| | | | | | CCCTAGCCCCTTGCAGTGATAAATA (SEQ ID NO: 4155) |
| | | | | | GTGTTTGGTGTTCTTATCTCCACCC (SEQ ID NO: 4156) |
| | | | | | TCAGACAGATGCCTCTTGCTTTTAA (SEQ ID NO: 4157) |
| | | | | | ACGTGTTGTAGGGTTCTTGGTCTGT (SEQ ID NO: 4158) |
| | | | | | GAGAAGGAAGTGAGCCCACTGCTCT (SEQ ID NO: 4159) |
| | | | | | ATGTGGGTGAGTCCACCAGAGGCCC (SEQ ID NO: 4160) |
| | | | | | CCAGCCCTCGAGGCAGTGTGTGTGG (SEQ ID NO: 4161) |
| | | | | | ATATGTACCCTGCACTCATGAATGT (SEQ ID NO: 4162) |
| | | | | | GAAGTATTTAACATTCTCCCATCCC (SEQ ID NO: 4163) |
| 208627_s_at | YBX1 | YB1; BP-8; | Y box binding protein 1 | 4904 | TAACCCCAGGAAGTACCTTCGCAGT (SEQ ID NO: 4164) |
| | | CSDB; DBPB; | | | GCGGAGGCAGCAAATGTTACAGGTC (SEQ ID NO: 4165) |
| | | YB-1; CSDA2; | | | GGTGGTGTTCCAGTTCAAGGCAGTA (SEQ ID NO: 4166) |
| | | NSEP1; NSEP-1; | | | GCAGACCGTAACCATTATAGACGCT (SEQ ID NO: 4167) |
| | | MDR-NF1; | | | GACGCTATCCACGTCGTAGGGGTCC (SEQ ID NO: 4168) |
| | | MGC104858; | | | GAGGGATCGGAGAGTGCTCCCGAAG (SEQ ID NO: 4169) |
| | | MGC110976; | | | GTTCCCACCTTACTACATGCGGAGA (SEQ ID NO: 4170) |
| | | MGC117250 | | | CTATGGGCGTCGACCACAGTATTCC (SEQ ID NO: 4171) |
| | | | | | TTCCAACCCTCCTGTGCAGGGAGAA (SEQ ID NO: 4172) |
| | | | | | GGGTGCTGACAACCAGGGTGCAGGA (SEQ ID NO: 4173) |
| | | | | | GGGATATAGACCACGATTCCGCAGG (SEQ ID NO: 4174) |
| 208628_s_at | YBX1 | YB1; BP-8; | Y box binding protein 1 | 4904 | AACTTCAATTACCGACGCAGACGCC (SEQ ID NO: 4175) |
| | | CSDB; DBPB; | | | GAAAACCCTAAACCACAAGATGGCA (SEQ ID NO: 4176) |
| | | YB-1; CSDA2; | | | AGAGACAAAAGCAGCCGATCCACCA (SEQ ID NO: 4177) |
| | | NSEP1; NSEP-1; | | | ATCCACCAGCTGAGAATTCGTCCGC (SEQ ID NO: 4178) |
| | | MDR-NF1; | | | TGAGTAAATGCCGGCTTACCATCTC (SEQ ID NO: 4179) |
| | | MGC104858; | | | ATCTCTACCATCATCCGGTTTAGTC (SEQ ID NO: 4180) |
| | | MGC110976; | | | TCATCCGGTTTAGTCATCCAACAAG (SEQ ID NO: 4181) |
| | | MGC117250 | | | GATTGGAGCTGAAGACCTAAAGTGC (SEQ ID NO: 4182) |
| | | | | | CCTAAAGTGCTTGCTTTTTGCCCGT (SEQ ID NO: 4183) |
| | | | | | CTTTTTGCCCGTTGACCAGATAAAT (SEQ ID NO: 4184) |
| | | | | | GCATTATCTATGCAGCATGGGGTTT (SEQ ID NO: 4185) |
| 200640_at | YWHAZ | KCIP-1, | tyrosine 3- | 7534 | ATACTCTTATAGCCTGCCTTCAATT (SEQ ID NO: 4186) |
| | | MGC111427, | monooxygenase/tryptophan | | TATTCTTTCTATTTGTCAGGTGCAC (SEQ ID NO: 4187) |
| | | MGC126532, | 5- | | GTCAGGTGCACAAGATTACCTTCCT (SEQ ID NO: 4188) |
| | | MGC138156 | monooxygenase | | CAAGATTACCTTCCTGTTTTAGCCT (SEQ ID NO: 4189) |
| | | | activation protein, | | CTTGTCACCAACCATTCTTACTTGG (SEQ ID NO: 4190) |
| | | | zeta polypeptide | | AACCATTCTTACTTGGTGGCCATGT (SEQ ID NO: 4191) |
| | | | | | AAAAAGGCCGCATGATCTTTCTGGC (SEQ ID NO: 4192) |
| | | | | | CTGGCTCCACTCAGTGTCTAAGGCA (SEQ ID NO: 4193) |
| | | | | | CCTCATCCTATTTACTGCAGCAAAT (SEQ ID NO: 4194) |
| | | | | | GCAAATCTCTCCTTAGTTGATGAGA (SEQ ID NO: 4195) |
| | | | | | GATGAGACTGTGTTTATCTCCCTTT (SEQ ID NO: 4196) |
| 205383_s_at | ZBTB20 | RP23-283M18.1, | zinc finger and BTB | 26137 | GGAGAGAAGTCCTACGAGTGCTACA (SEQ ID NO: 4197) |
| | | 1300017A20Rik, | domain containing 20 | | AAAGAAGTTCTCTCACAAGACCCTC (SEQ ID NO: 4198) |
| | | 7330412A13Rik, | | | CTCCGTCTGCCCAGCAAAGTTTGAC (SEQ ID NO: 4199) |
| | | A930017C21Rik, | | | CGAGCAGTTCAACGACCACATGAGG (SEQ ID NO: 4200) |
| | | D16Wsu73e, | | | TGAAGAACTGTTTTTTGCCTGCTGG (SEQ ID NO: 4201) |
| | | DPZF, HOF, | | | GGTACATTACATTTCCGGAGGCTTG (SEQ ID NO: 4202) |
| | | ODA-8S, Oda8, | | | GAATAATAGTTTTCCCAGTCTCCCT (SEQ ID NO: 4203) |
| | | Zfp288 | | | CCCTCGGATGGTGGCCTTAAGGCCT (SEQ ID NO: 4204) |
| | | | | | GCCTTAAGGCCTGGTAGTGCTTCAA (SEQ ID NO: 4205) |
| | | | | | GAGGTCCACTGGTTGGATCTCTAGC (SEQ ID NO: 4206) |
| | | | | | ATCTCTAGCTACTGGCCTCTAAATA (SEQ ID NO: 4207) |
| 206169_x_at | ZC3H7B | DKFZp434K0920, | zinc finger CCCH- | 23264 | CATGGTGGCATGCTTCTGTAATCCC (SEQ ID NO: 4208) |
| | | FLJ13787, | type containing 7B | | ATGGTGGCATGCTTCTGTAATCCCA (SEQ ID NO: 4209) |
| | | KIAA1031, | | | GCTACTCAGGAGGCCGAGATTAGAG (SEQ ID NO: 4210) |
| | | RoXaN | | | GAGGCCGAGATTAGAGAATCACTTG (SEQ ID NO: 4211) |
| | | | | | GAATCACTTGAATGTGGGAGTCGGA (SEQ ID NO: 4212) |
| | | | | | CTTGAATGTGGGAGTCGGAGGTTGT (SEQ ID NO: 4213) |
| | | | | | GAATGTGGGAGTCGGAGGTTGTAAT (SEQ ID NO: 4214) |
| | | | | | GTGGGAGTCGGAGGTTGTAATGAGC (SEQ ID NO: 4215) |
| | | | | | GGAGTCGGAGGTTGTAATGAGCTGA (SEQ ID NO: 4216) |
| | | | | | GTCGGAGGTTGTAATGAGCTGAGAT (SEQ ID NO: 4217) |
| | | | | | GAGGTTGTAATGAGCTGAGATCACA (SEQ ID NO: 4218) |
| 202778_s_at | ZMYM2 | FIM, MYM, | zinc finger, MYM-type 2 | 7750 | GAGTGAAAGTTGCCATTATTCTATG (SEQ ID NO: 4219) |
| | | RAMP, SCLL, | | | GAGAATGTGCCATGTATAATTTTTT (SEQ ID NO: 4220) |
| | | ZNF198 | | | ACATCCATATTGCACAACTCTACTG (SEQ ID NO: 4221) |
| | | | | | AACTCTACTGTTGCAAAGCTTCCTT (SEQ ID NO: 4222) |
| | | | | | TCCTTGGAAGGGGGCTCTTTTACTG (SEQ ID NO: 4223) |
| | | | | | TACTGGGTTCTTAACCAGATGGTTG (SEQ ID NO: 4224) |
| | | | | | GGTTGTGTATGGGTAGCACTACTAA (SEQ ID NO: 4225) |
| | | | | | GAACTTGCAGTGTCTTTCGGAATTT (SEQ ID NO: 4226) |
| | | | | | ATTTTAGTTACTGAAGCCTTACAAG (SEQ ID NO: 4227) |
| | | | | | GAGAGATACCATCTTCTGTACCAAA (SEQ ID NO: 4228) |
| | | | | | GTAATGTGAATCTATGCTGGTGAAA (SEQ ID NO: 4229) |
| 215948_x_at | ZMYM5 | MYM; ZNF237; | zinc finger, MYM-type 5 | 9205 | ATACCAACTTGGGAGATGTCGCTAA (SEQ ID NO: 4230) |
| | | HSPC050; | | | GATGTCGCTAAAGGACTTCAGTCAA (SEQ ID NO: 4231) |
| | | ZNF198L1 | | | AATATACAAACGTACACCCCATCTT (SEQ ID NO: 4232) |
| | | | | | CCATCTTTAACTCCACAGACCAAGA (SEQ ID NO: 4233) |
| | | | | | GAATGTGGCAGGAGACTTATTTCAG (SEQ ID NO: 4234) |
| | | | | | GAATTTGCAACTCATCATAGTCCTG (SEQ ID NO: 4235) |
| | | | | | AAGGCTAATGTCATTCTTCCAGTAG (SEQ ID NO: 4236) |
| | | | | | GAATCAAGCAAATCCTTCCAAGAAT (SEQ ID NO: 4237) |
| | | | | | ATCTTGTTTGTCTCCCTGTGAAAAC (SEQ ID NO: 4238) |
| | | | | | ACTCTTTCATCTACATAATGCACGT (SEQ ID NO: 4239) |
| | | | | | TAATGCACGTGTTCTGGATGTATAA (SEQ ID NO: 4240) |
| 207605_x_at | ZNF117 | H-plk, HPF9, | zinc finger protein | 51351 | GCGGCAAAGCTTTTAACCGATCCTC (SEQ ID NO: 4241) |
| | | MGC22613 | 117 | | ACCGATCCTCAAAACTTACTGAACA (SEQ ID NO: 4242) |
| | | | | | GTGACAAGGCCTTTAACCTGTCCTC (SEQ ID NO: 4243) |
| | | | | | CCTGTCCTCAACGCTTACTAAACAT (SEQ ID NO: 4244) |
| | | | | | TAAACAGTTCTCACACCTTGCTATA (SEQ ID NO: 4245) |
| | | | | | TCTCACACCTTGCTATACATAATAT (SEQ ID NO: 4246) |
| | | | | | GGCAAAGCTTTTAACTCATCCTCAA (SEQ ID NO: 4247) |
| | | | | | TCATCCTCAAACCTTACTGCACATA (SEQ ID NO: 4248) |
| | | | | | GCAAAGCTAACCTATCATCAACCCT (SEQ ID NO: 4249) |
| | | | | | CAACCCTTACTCCACATAAGACAAT (SEQ ID NO: 4250) |
| | | | | | AATGCTTGTAACTAAATCTCAAACT (SEQ ID NO: 4251) |
| 219123_at | ZNF232 | ZSCAN11 | zinc finger protein | 7775 | TGGTCCCCTGCCCAGGAGGAAAGTT (SEQ ID NO: 4252) |
| | | | 232 | | TATAACTCACATCTTGTTGTCCACC (SEQ ID NO: 4253) |
| | | | | | CACATCTTGTTGTCCACCAGAGAGT (SEQ ID NO: 4254) |
| | | | | | AAACAGAGCTCAAACCTCGGTCAGC (SEQ ID NO: 4255) |
| | | | | | AAGGCCTTCAGATGGGGTGCTCATC (SEQ ID NO: 4256) |
| | | | | | ATGGGGTGCTCATCTTGTTCAGCAT (SEQ ID NO: 4257) |
| | | | | | AGCTCATATCTAAGTCAGCATCGGA (SEQ ID NO: 4258) |
| | | | | | GGTGCTCAGAGCTCATTAGACATCG (SEQ ID NO: 4259) |
| | | | | | ACATCGGAGAGTTCATGCCAGAAAA (SEQ ID NO: 4260) |
| | | | | | TGCCAGAAAAGAGCCTTCCCATTGA (SEQ ID NO: 4261) |
| | | | | | GAATTCTACATCGGTCTAATCTACT (SEQ ID NO: 4262) |
| 206829_x_at | ZNF430 | DKFZp762K012, | zinc finger protein | 80264 | GATAAAGCCTTTAGCCAGTCTTCAA (SEQ ID NO: 4263) |
| | | FLJ13659, | 430 | | AGCCAGTCTTCAACTCTTACTAAAC (SEQ ID NO: 4264) |
| | | MGC133232 | | | GGAGAGAAACCCTACAACTGTGAAG (SEQ ID NO: 4265) |
| | | | | | ATACGGCAAAGCTTTCAACCAGTCC (SEQ ID NO: 4266) |
| | | | | | TTCAACCAGTCCTCAAACCTTATTG (SEQ ID NO: 4267) |
| | | | | | ATTGTGGCAAAGCCTCTAACCCGTC (SEQ ID NO: 4268) |
| | | | | | CCCGTCCTGAATTCTTACTAAACAT (SEQ ID NO: 4269) |
| | | | | | ATGGCAAAGCCTTCAACAAGTCCTC (SEQ ID NO: 4270) |
| | | | | | ACAAGTCCTCAATTCTTACCAGACA (SEQ ID NO: 4271) |
| | | | | | AGATAATTCTGGCTGGGTGCGGTGG (SEQ ID NO: 4272) |
| | | | | | GGCTGAGACGGGTGAATTACATGAG (SEQ ID NO: 4273) |

All gene probe set identification numbers and Entrez Gene identification numbers of Table 1 are hereby incorporated by reference in their entirety

The term “prognosis” as used in the context of the present invention refers to the prediction of disease outcome for a subject having colon cancer. Disease outcome encompasses disease progression, reoccurrence, metastasis, and drug resistance. Determining the prognosis of a subject having colon cancer in accordance with the methods of the present invention has particular value for determining an appropriate treatment plan. For example, the prognosis of a subject determined using the methods of the present invention can predict a subject's response to a specific drug or combination of drugs, chemotherapy, radiation therapy, or surgical removal, and whether survival after following the administration of a particular treatment plan is likely.

As used herein a “disease prognosis expression profile” refers to gene expression of a collection of genes informative of disease outcome that is associated with a good disease outcome or a bad disease outcome. The gene expression of a collection of genes that is associated with a good disease outcome is a good disease prognosis expression profile. A good disease prognosis expression profile consists of genes having expression levels that are below the average tumor sample expression level and/or genes having expression levels that are above the average tumor sample expression level. In a preferred embodiment of the present invention a good disease prognosis expression profile for the group of 176 genes informative of colon cancer prognosis consists of genes having expression levels that are below that of an average tumor sample expression level that are selected from the group consisting of AK023058*, AIP, ARL2BP, C1GALT1, CDC42BPA, C8orf70, CLN5, COBL, CYB5B, MOSPD1, DOCK9, EGFR, FKBP14, DND1, GREM2, GPR177, GALNS, GRB10, GRP, GSTA1, RP3-377H14.5, HOXB7, ZNF117, TNIK, LANCL1, METRN, LEPREL1, NAB1, NISCH, OGT, OSBPL3, PDGFA, PRDM2, PRELP, PSPC1, RECQL, RYK, SMURF2, TLN1, UNC84A, USP12, ZMYM2, ZMYM5, AL359599*, ARL4A, N4BP2L2, GLS, C19orf36, TMCC1, METTL3, TMEM16A, RTN2, SCAMP1, SF3B1, SOX4, STK3, ZNF430, C6orf15, C7orf10, CHST12, ETV1, ACSL4, FLJ10357, C5orf23, AA058828*, CDR2L, KLC1, MAP4, NUMB, PAM, PGDS, PTHLH, ZC3H7B, SAV1, SGCD, SYNGR1, TES, IFT88, TRIM36 and VPS41. The good disease prognosis expression profile for the group of 176 genes further consists of genes having expression levels that are above the average tumor sample expression level that are selected from the group consisting of SERPINA1, RPLP0, RPLP0-like, CYB561, AKR1A1, AP3D1, ARL6IP4, OGFOD2, ASNA1, CFB, ERP29, SMG7, CASP1, CCNA2, LOC100131861, SAMM50, COX5A, CXCL11, DAZAP2, DDX23, FDFT1, COMMD4, GCHFR, GRHPR, GSR, ISG20, ITGAE, KIAA0746, SERINC2, FRYL, LRRC47, LAMP3, R3HCC1, MAPKAPK5, MCM5, MCRS1, TMEM106C, MMP3, MTUS1, LRRC41, NAT1, NDUFC1, YBX1, PEBP1, PIGR, PSMA5, SFPQ, SLC25A3, SLC39A8, SQRDL, SRP72, SSNA1, TAPBPL, TEGT, PBK, UCP2, UQCRH, XPO7, CCT2, CNOT7, DHX15, TMEM87A, ELP3, FAM82C, LL22NC03-5H6.5, DENND2D, WDR68, IL15RA, DENND2A, KIF13B, MFHAS1, SPATA5L1, MYRIP, PIGT, PSMD9, RPS27L, TEGT, TNFRSF10B, UBE2L6, USP3, ATP5B, CALML4, C1orf144, TMEM33, C12orf52, GHITM, H2AFZ, LAP3, MRPL46, SORD, CNPY2, TNFSF10, U2AF2, CMPK1, UQCRFS1, WARS and WDR1.

The gene expression of a collection of genes informative of disease outcome that is associated with a bad disease outcome is a bad disease prognosis expression profile. A bad disease prognosis expression profile consists of genes having expression levels above and/or below the average tumor sample expression level. In a preferred embodiment of the present invention, a bad disease prognosis expression file for the collection of 176 genes informative of colon cancer prognosis consists of genes having expression levels that are below that of an average tumor sample expression level selected from the group consisting of SERPINA1, RPLP0, RPLP0-like, CYB561, AKR1A1, AP3D1, ARL6IP4, OGFOD2, ASNA1, CFB, ERP29, SMG7, CASP1, CCNA2, LOC100131861, SAMM50, COX5A, CXCL11, DAZAP2, DDX23, FDFT1, COMMD4, GCHFR, GRHPR, GSR, ISG20, ITGAE, KIAA0746, SERINC2, FRYL, LRRC47, LAMP3, R3HCC1, MAPKAPK5, MCM5, MCRS1, TMEM106C, MMP3, MTUS1, LRRC41, NAT1, NDUFC1, YBX1, PEBP1, PIGR, PSMA5, SERPINA1, SFPQ, SLC25A3, SLC39A8, SQRDL, SRP72, SSNA1, TAPBPL, TEGT, PBK, UCP2, UQCRH, XPO7, CCT2, CNOT7, DHX15, TMEM87A, ELP3, FAM82C, LL22NC03-5H6.5, DENND2D, WDR68, IL15RA, DENND2A, KIF13B, MFHAS1, SPATA5L1, MYRIP, PIGT, PSMD9, RPS27L, TNFRSF10B, UBE2L6, USP3, ATP5B, CALML4, C1orf144, TMEM33, C12orf52, GHITM, H2AFZ, LAP3, MRPL46, SORD, CNPY2, TNFSF10, U2AF2, CMPK1, UQCRFS1, WARS and WDR; and genes having expression levels that are above the average tumor sample expression level selected from the group consisting of AK023058*, AIP, ARL2BP, C1GALT1, CDC42BPA, C8orf70, CLN5, COBL, CYB5B, MOSPD1, DOCK9, EGFR, FKBP14, DND1, GREM2, GPR177, GALNS, GRB10, GRP, GSTA1, RP3-377H14.5, HOXB7, ZNF117, TNIK, LANCL1, METRN, LEPREL1, NAB1, NISCH, OGT, OSBPL3, PDGFA, PRDM2, PRELP, PSPC1, RECQL, RYK, SMURF2, TLN1, UNC84A, USP12, ZMYM2, ZMYM5, AL359599*, ARL4A, N4BP2L2, GLS, C19orf36, TMCC1, METTL3, TMEM16A, RTN2, SCAMP1, SF3B1, SOX4, STK3, ZNF430, C6orf15, C7orf10, CHST12, ETV1, ACSL4, FLJ10357, C5orf23, AA058828*, CDR2L, KLC1, MAP4, NUMB, PAM, PGDS, PTHLH, ZC3H7B, SAV1, SGCD, SYNGR1, TES, IFT88, TRIM36 and VPS41.

Another aspect of the present invention relates to a method for determining the prognosis of a subject having colon cancer that involves obtaining a biological sample from the subject and detecting the expression levels of at least five genes selected from the group of 101 genes informative of colon cancer prognosis. The group of 101 genes informative of colon cancer prognosis are provided in Table 2 below. This method further involves comparing the detected expression levels of the at least five genes from the biological sample with the expression levels of the corresponding at least five genes when associated with a good disease prognosis expression profile and when associated with a bad disease prognosis expression profile. Based on that comparison, the prognosis of the subject having colon cancer is determined.

TABLE 2
101 Genes Informative of Colon Cancer Prognosis

| | | | | Accession | | |
|---|---|---|---|---|---|---|
| | | | | # | | |
| Probe Set | Gene | | | (Entrez | | |
| ID | Symbol | AKA | Gene Title | Gene ID) | Probe Sequence(s) | |
| 200027_at | NARS | ASNRS, | asparaginyl- | 4677 | ATCTTTTCTAACTCTGCTTAGCTGC | (SEQ ID NO: 4274) |
| | | NARS1 | tRNA | | AGCTGCTAATAATCCTGAGGCATAG | (SEQ ID NO: 4275) |
| | | | synthetase | | TAGTGATTCACAGTATCCCTCTTAG | (SEQ ID NO: 4276) |
| | | | | | AGTATCCCTCTTAGCATTAATTTAA | (SEQ ID NO: 4277) |
| | | | | | GAGGCAGATTGATTTTCCCTCTTTC | (SEQ ID NO: 4278) |
| | | | | | CCCACCATGTCCTTAGATCTAATCT | (SEQ ID NO: 4279) |
| | | | | | TAGATCTAATCTGTGCTACCTTATT | (SEQ ID NO: 4280) |
| | | | | | GCTACCTTATTAACTCACAGCAGGC | (SEQ ID NO: 4281) |
| | | | | | ACAGCAGGCTTACTGAATGGCTTCA | (SEQ ID NO: 4282) |
| | | | | | CAGATTTAGTTGATTTCTCCACCAA | (SEQ ID NO: 4283) |
| | | | | | GCATGTCATGTATTCTCAATAGGCT | (SEQ ID NO: 4284) |
| 200609_s_{—} | WDR1 | API, NORI-1 | WD repeat | 9948 | GGCTCTCACTATCTGTCAGGGGAGG | (SEQ ID NO: 4285) |
| at | | | domain 1 | | GCAAAGCCGGAATTCTGTGTTGAAC | (SEQ ID NO: 4286) |
| | | | | | TGAACAGATGTCTGTCTCCCTAGTG | (SEQ ID NO: 4287) |
| | | | | | AGTGTGTGACTCACACCTTGTGGCT | (SEQ ID NO: 4288) |
| | | | | | CGCCACCTCCAGATCAGATGGGGAC | (SEQ ID NO: 4289) |
| | | | | | GACACACAACCCCTGGATATGTTTC | (SEQ ID NO: 4290) |
| | | | | | GTTGCCTGTCAGTGTTTACAAACTA | (SEQ ID NO: 4291) |
| | | | | | TGACGGCACCGTGTCCAAGTTTTTA | (SEQ ID NO: 4292) |
| | | | | | TTTAGAACCCTTGTTAGCCAGACCG | (SEQ ID NO: 4293) |
| | | | | | TAGCCAGACCGAGGTGTCCTGGTCA | (SEQ ID NO: 4294) |
| | | | | | ACCATCATGCTTTGATGTTCCCCTG | (SEQ ID NO: 4295) |
| 200629_at | WARS | GAMMA-2, | tryptophanyl- | 7453 | TCCAGTTTACTGAACTCCAGACCAT | (SEQ ID NO: 4296) |
| | | IF153, IFP53 | tRNA | | CCAGACCATGCATGTAGTCCACTCC | (SEQ ID NO: 4297) |
| | | | synthetase | | TGCAGAGTCAGGGTGGCTTTTCCCC | (SEQ ID NO: 4298) |
| | | | | | CTGTGTCCGATGCCAAGGAGTCCTG | (SEQ ID NO: 4299) |
| | | | | | GGACGGGTGGACTTAGCTAGGGAGA | (SEQ ID NO: 4300) |
| | | | | | AAGTCGAGGCAGCAGCCCTCGAGGC | (SEQ ID NO: 4301) |
| | | | | | TCGAGGCCCTCACAGATGTCTAGGC | (SEQ ID NO: 4302) |
| | | | | | GTCTAGGCAGGCCTCATTTCATCAC | (SEQ ID NO: 4303) |
| | | | | | ATTTCATCACGCAGCATGTGCAGGC | (SEQ ID NO: 4304) |
| | | | | | ATCTCAGGGAAGTCCTTGGTTGATG | (SEQ ID NO: 4305) |
| | | | | | TTGGTTGATGTATCTGGGTCTCCTC | (SEQ ID NO: 4306) |
| 200877_at | CCT4 | CCT-DELTA, | chaperonin | 10575 | GTGGTATGGAATCCTACTGCGTTCG | (SEQ ID NO: 4307) |
| | | Cctd, | containing | | GCGTTCGTGCTTTTGCAGATGCTAT | (SEQ ID NO: 4308) |
| | | MGC126164, | TCP1, | | GTCATTCCATCTACACTAGCTGAAA | (SEQ ID NO: 4309) |
| | | MGC126165, | subunit 4 | | CTAGCTGAAAATGCCGGCCTGAATC | (SEQ ID NO: 4310) |
| | | SRB | (delta) | | GGCCTGAATCCCATTTCTACAGTAA | (SEQ ID NO: 4311) |
| | | | | | GAACTAAGAAACCGGCATGCCCAGG | (SEQ ID NO: 4312) |
| | | | | | TGGAGGAACTGGTTGTCCAGCCTCT | (SEQ ID NO: 4313) |
| | | | | | CCAGCCTCTGTTGGTATCAGTCAGT | (SEQ ID NO: 4314) |
| | | | | | CAGTGCTCTGACTCTTGCAACTGAA | (SEQ ID NO: 4315) |
| | | | | | GACTAGCACCATTATGATCACCAGT | (SEQ ID NO: 4316) |
| | | | | | TCTGGGCTTGGTCTTCCAGTTGGCA | (SEQ ID NO: 4317) |
| 201322_at | ATP5B | ATPMB, | ATP | 506 | TTGGATGCTACCACTGTACTGTCGC | (SEQ ID NO: 4318) |
| | | ATPSB, | synthase, H+ | | TCGCGTGCCATTGCTGAGCTGGGCA | (SEQ ID NO: 4319) |
| | | MGC5231 | transporting, | | AGCTGTGGATCCTCTAGACTCCACC | (SEQ ID NO: 4320) |
| | | | mitochondrial | | GTGAGCATTACGATGTTGCCCGTGG | (SEQ ID NO: 4321) |
| | | | F1 complex, | | TCCTGCAGGACTACAAATCCCTCCA | (SEQ ID NO: 4322) |
| | | | beta | | GAAGACAAGTTGACCGTGTCCCGTG | (SEQ ID NO: 4323) |
| | | | polypeptide | | TTGTCTCAGCCATTCCAGGTTGCTG | (SEQ ID NO: 4324) |
| | | | | | GAGGTCTTCACAGGTCATATGGGGA | (SEQ ID NO: 4325) |
| | | | | | GAAGCTGGTACCCCTGAAGGAGACC | (SEQ ID NO: 4326) |
| | | | | | ACAGGCCTTCTATATGGTGGGACCC | (SEQ ID NO: 4327) |
| | | | | | TCGTGAGGGGTCTTTGTCCTCTGTA | (SEQ ID NO: 4328) |
| 201562_s_{—} | SORD | SORD1 | sorbitol | 6652 | GATGTTAATGGGCTCTGCTCATCCC | (SEQ ID NO: 4329) |
| at | | | dehydrogenase | | GGGTGGGCTCTGATGCAGAACTTTC | (SEQ ID NO: 4330) |
| | | | | | GGAACTTGTAGCCAGAATGCCCTGT | (SEQ ID NO: 4331) |
| | | | | | GAATGCCCTGTTCATGCTGAGCAAA | (SEQ ID NO: 4332) |
| | | | | | TTCTTCCTGGAGTGCCTTCATTGAG | (SEQ ID NO: 4333) |
| | | | | | TCCACTGCTACTGACCCAGAGGGGA | (SEQ ID NO: 4334) |
| | | | | | TCATGAAGACTTAACTGGCCCAGAA | (SEQ ID NO: 4335) |
| | | | | | AAAATCTGCCACTCAGGGTCTGGGA | (SEQ ID NO: 4336) |
| | | | | | GGGTCTGGGATGAAGGCTTGTCAGC | (SEQ ID NO: 4337) |
| | | | | | GCTTGTCAGCACTTCCAGTTTAGAA | (SEQ ID NO: 4338) |
| | | | | | GAAAAGGCATCACTTTCCTGGATCC | (SEQ ID NO: 4339) |
| 201646_at | UBE2L6 | MGC40331, | ubiquitin- | 9246 | GTGTGTTGTGTATGGACTCACTCCC | (SEQ ID NO: 4340) |
| | | RIG-B, UBCH8 | conjugating | | CCTTTACATTCCCCAGAGCCAAGGG | (SEQ ID NO: 4341) |
| | | | enzyme E2L6 | | GTTTGCAGTTACAGGCCAGTTCTCC | (SEQ ID NO: 4342) |
| | | | | | TTCTCCAGCTCTCCATCTTAGAGAG | (SEQ ID NO: 4343) |
| | | | | | TGAATCCAGCAGCCAACTCGAATCC | (SEQ ID NO: 4344) |
| | | | | | CCCCCTAGGGCTCAGGCACTGAGGG | (SEQ ID NO: 4345) |
| | | | | | GGAGGGTACCCTATTTACAACTGAG | (SEQ ID NO: 4346) |
| | | | | | GAGTCAGCCAAGCCACTGATGGGAA | (SEQ ID NO: 4347) |
| | | | | | GTATATATCCTCCAGCATTCAGTCC | (SEQ ID NO: 4348) |
| | | | | | CCAGGGGGAGCCACGGAAACCATGT | (SEQ ID NO: 4349) |
| | | | | | GAAACCATGTTCTTGCTTAAGCCAT | (SEQ ID NO: 4350) |
| 201762_s_{—} | PSME2 | PA28B, | proteasome | 5721 | CAACACCTGATCCCCAAGATTGAAG | (SEQ ID NO: 4351) |
| at | | PA28beta, | (prosome, | | GGGGTAGCAATCCAGGAGAAGGTGC | (SEQ ID NO: 4352) |
| | | REGbeta | macropain) | | GAGAGGGTGAATGCCGTCAAGACCA | (SEQ ID NO: 4353) |
| | | | activator | | GAAGCTTTCCAGACAACCATTTCCA | (SEQ ID NO: 4354) |
| | | | subunit 2 | | GACAACCATTTCCAAGTACTTCTCA | (SEQ ID NO: 4355) |
| | | | (PA28 beta) | | GGCCTCCAAGGAGACTCATGTAATG | (SEQ ID NO: 4356) |
| | | | | | AATGGATTACCGGGCCTTGGTGCAT | (SEQ ID NO: 4357) |
| | | | | | GCCTTGGTGCATGAGCGAGATGAGG | (SEQ ID NO: 4358) |
| | | | | | TATGGGGAGCTCAGGGCCATGGTGC | (SEQ ID NO: 4359) |
| | | | | | GGGCCTTCTATGCTGAGCTTTATCA | (SEQ ID NO: 4360) |
| | | | | | CTTTATCATATCATCAGCAGCAACC | (SEQ ID NO: 4361) |
| 201782_s_{—} | AIP | ARA9, | aryl | 9049 | GTGCCACTTATCCACCAGGAGGGCA | (SEQ ID NO: 4362) |
| at | | FKBP16, | hydrocarbon | | AGGAGGCTGCTGCCAAGTACTACGA | (SEQ ID NO: 4363) |
| | | FKBP37, | receptor | | CAAGTACTACGATGCCATTGCCTGC | (SEQ ID NO: 4364) |
| | | SMTPHN, | interacting | | CATTGCCTGCCTCAAGAACCTGCAG | (SEQ ID NO: 4365) |
| | | XAP2 | protein | | CCTGGGTCCCCTGAATGGATCCAGC | (SEQ ID NO: 4366) |
| | | | | | GAGGAGTACTACGAGGTGCTGGACC | (SEQ ID NO: 4367) |
| | | | | | GTCAAGGCCTACTTCAAGCGGGGCA | (SEQ ID NO: 4368) |
| | | | | | CGGCCGTGTGGAATGCCCAGGAGGC | (SEQ ID NO: 4369) |
| | | | | | GCCTGTGGTGAGCCGAGAGCTGCGG | (SEQ ID NO: 4370) |
| | | | | | CCTGGAGGCACGGATCCGGCAGAAG | (SEQ ID NO: 4371) |
| | | | | | GGGATCTTCTCCCATTGACAGGAGC | (SEQ ID NO: 4372) |
| 201890_at | RRM2 | R2, RR2M | ribonucleotide | 6241 | GCTACTTTGAATTAATCTGCCTTTA | (SEQ ID NO: 4373) |
| | | | reductase | | TTGATCTTTTGGCCCCATTTGTTAA | (SEQ ID NO: 4374) |
| | | | M2 | | TTGGCCCCATTTGTTAATTGTATTC | (SEQ ID NO: 4375) |
| | | | polypeptide | | GTATTCAGTATTTGAACGTCGTCCT | (SEQ ID NO: 4376) |
| | | | | | GAACGTCGTCCTGTTTATTGTTAGT | (SEQ ID NO: 4377) |
| | | | | | GTTAATCCAGATTATATGGTCCTTA | (SEQ ID NO: 4378) |
| | | | | | TATGGTCCTTATATGTGTACAACAT | (SEQ ID NO: 4379) |
| | | | | | GTCTTGCATTGTGAGGTACAGGCGG | (SEQ ID NO: 4380) |
| | | | | | GTACAGGCGGAAGTTGGAATCAGGT | (SEQ ID NO: 4381) |
| | | | | | TTAGGATTCTGTCTCTCATTAGCTG | (SEQ ID NO: 4382) |
| | | | | | ATGTGAGGATTAACTTCTGCCAGCT | (SEQ ID NO: 4383) |
| 201932_at | LRRC41 | MGC126571, | leucine rich | 10489 | CTGGCTGGACCAGGATGCAGTCACA | (SEQ ID NO: 4384) |
| | | MGC126573, | repeat | | GGCTACCTGCCATGTGGTTAGCGAC | (SEQ ID NO: 4385) |
| | | MUF1, | containing 41 | | TGGTTAGCGACTCATGGACTCATCC | (SEQ ID NO: 4386) |
| | | PP7759, RP4- | | | TCCCAGGCCTTCGCAGATTATGTTA | (SEQ ID NO: 4387) |
| | | 636H5.2 | | | CGGTACCATCAGCTTGCAGGGGCTG | (SEQ ID NO: 4388) |
| | | | | | TGCAGGGGCTGAAGCATGGGCTGCC | (SEQ ID NO: 4389) |
| | | | | | GAGGCCCAGCAAACAGGCATTCTCA | (SEQ ID NO: 4390) |
| | | | | | GGCATTCTCACAGCTGGGTTTATAG | (SEQ ID NO: 4391) |
| | | | | | CTCAGTATCCTGGGAACCCTGGGCC | (SEQ ID NO: 4392) |
| | | | | | ATAATTGCTGAAGAGATCCCCTCCC | (SEQ ID NO: 4393) |
| | | | | | TCAAACCCTTTCATACCTTTATTCT | (SEQ ID NO: 4394) |
| 201947_s_{—} | CCT2 | 99D8.1, CCT- | chaperonin | 10576 | GAAGCTGTTGCAATGGAGTCTTATG | (SEQ ID NO: 4395) |
| at | | beta, CCTB, | containing | | ATGTTGCCAACCATCATAGCTGACA | (SEQ ID NO: 4396) |
| | | MGC142074, | TCP1, | | GCTATGACAGTGCAGACCTGGTGGC | (SEQ ID NO: 4397) |
| | | MGC142076, | subunit 2 | | GCTCAGGGCTGCTCACAGTGAAGGC | (SEQ ID NO: 4398) |
| | | PRO1633, | (beta) | | GAAGGCAATACCACTGCTGGATTGG | (SEQ ID NO: 4399) |
| | | TCP-1-beta | | | AGATATGGCTATCCTGGGTATAACA | (SEQ ID NO: 4400) |
| | | | | | AGGTGATTCTGCGTGTGGACAACAT | (SEQ ID NO: 4401) |
| | | | | | GGACAACATCATCAAAGCGGCACCC | (SEQ ID NO: 4402) |
| | | | | | ACGTGCTGTCGATCTTTGGACCAGT | (SEQ ID NO: 4403) |
| | | | | | GACCAGTTTCTAGCAAAGTTGTGTT | (SEQ ID NO: 4404) |
| | | | | | ATTTAGCTGACCTTCGCTTTAACAT | (SEQ ID NO: 4405) |
| 202168_at | TAF9 | AD-004, AK6, | TAF9 RNA | 6880 | AAAAGGCATCAACTTCTGCGGGAAG | (SEQ ID NO: 4406) |
| | | CGI-137, | polymerase | | GTCCCGCGGTTAAGTGTTGGTTCAG | (SEQ ID NO: 4407) |
| | | CINAP, CIP, | II, TATA box | | TACTAGCAGACCAAGTACTCCCACA | (SEQ ID NO: 4408) |
| | | MGC1603, | binding | | AAAGTAGGGACTCCCATGTCCCTCA | (SEQ ID NO: 4409) |
| | | MGC3647, | protein | | AGTACAGATGCCTACTTCTCAGTCT | (SEQ ID NO: 4410) |
| | | MGC5067, | (TBP)- | | CTACTTCTCAGTCTCCAGCTGTAAA | (SEQ ID NO: 4411) |
| | | MGD: 1603, | associated | | GTAAAAGCTTCAATTCCTGCAACCT | (SEQ ID NO: 4412) |
| | | MGC: 3647, | factor, 32 kDa | | AATCCATCATTAATCGGGTCCAAAA | (SEQ ID NO: 4413) |
| | | MGC:5067, | | | GTCCAAAAACATTCTTATTACCACT | (SEQ ID NO: 4414) |
| | | TAF2G, | | | GATAATCTGTAATCTAGCCTTGCTG | (SEQ ID NO: 4415) |
| | | TAFII31, | | | AACACAGTTACCATACTTTTCAATT | (SEQ ID NO: 4416) |
| | | TAFII32, | | | | |
| | | TAFIID32 | | | | |
| 202455_at | HDAC5 | FLJ90614, | histone | 10014 | ACGAGTTCTCACCTGATGTGGTCCT | (SEQ ID NO: 4417) |
| | | HD5, NY-CO-9 | deacetylase | | GTCACCGCCAGATGTTTTGGCCACT | (SEQ ID NO: 4418) |
| | | | 5 | | TTTGGCCACTTGACCAGGCAGCTGA | (SEQ ID NO: 4419) |
| | | | | | CTGATGACCCTGGCAGGGGGCCGGG | (SEQ ID NO: 4420) |
| | | | | | GTCTCGGCTCTGCTCAGTGTAGAGC | (SEQ ID NO: 4421) |
| | | | | | GCAGCCCTTGGATGAGGCAGTCTTG | (SEQ ID NO: 4422) |
| | | | | | AAAGCCCAACATCAACGCAGTGGCC | (SEQ ID NO: 4423) |
| | | | | | CGCAGTGGCCACGCTAGAGAAAGTC | (SEQ ID NO: 4424) |
| | | | | | GTCATCGAGATCCAGAGCAAACACT | (SEQ ID NO: 4425) |
| | | | | | AAGCAGGTGAGACCGAGGAGGCCGA | (SEQ ID NO: 4426) |
| | | | | | CATGGAGCAGGAGCCTGCCCTGTGA | (SEQ ID NO: 4427) |
| 202565_s_{—} | SVIL | RP11- | supervillin | 6840 | ATATGTATTCATTCACTTTCAAGAT | (SEQ ID NO: 4428) |
| at | | 534G20.1, | | | CTTCTGTTGAATTAGCTCTGCCACC | (SEQ ID NO: 4429) |
| | | DKFZp686A17191 | | | AGCTCTGCCACCAATATGTATCTTC | (SEQ ID NO: 4430) |
| | | | | | GTATCTTCATACACGTTTGGAAATG | (SEQ ID NO: 4431) |
| | | | | | GGAAATGTTTCCTGCAGCATTAGGT | (SEQ ID NO: 4432) |
| | | | | | GCATTAGGTATGACTTGTTCTGAGT | (SEQ ID NO: 4433) |
| | | | | | TGAGTACTGCTTCCGGTGCTAAAAT | (SEQ ID NO: 4434) |
| | | | | | TTAATGGCATGGACTCTGGAGAATC | (SEQ ID NO: 4435) |
| | | | | | TATGCGAATCAACCTTTCTACCTTA | (SEQ ID NO: 4436) |
| | | | | | TCTACCTTAATATCTCCCCAAAAAT | (SEQ ID NO: 4437) |
| | | | | | GTTTGCTCTGCATTTTTGATGATGG | (SEQ ID NO: 4438) |
| 202705_at | CCNB2 | HsT17299 | cyclin B2 | 9133 | GCCACTACACTTCTTAAGGCGAGCA | (SEQ ID NO: 4439) |
| | | | | | GCGAGCATCAAAAGCCGGGGAGGTT | (SEQ ID NO: 4440) |
| | | | | | ATGGAGCTGACTCTCATCGACTATG | (SEQ ID NO: 4441) |
| | | | | | ATATGGTGCATTATCATCCTTCTAA | (SEQ ID NO: 4442) |
| | | | | | ATCCTTCTAAGGTAGCAGCAGCTGC | (SEQ ID NO: 4443) |
| | | | | | ACTTAACTAAATTCATCGCCATCAA | (SEQ ID NO: 4444) |
| | | | | | CAAAAGCCGTCAAAGACCTTGCCTC | (SEQ ID NO: 4445) |
| | | | | | CTTGCCTCCCCACTGATAGGAAGGT | (SEQ ID NO: 4446) |
| | | | | | GATAGGAAGGTCCTAGGCTGCCGTG | (SEQ ID NO: 4447) |
| | | | | | GATTTTGTACATAGTCCTCTGGTCT | (SEQ ID NO: 4448) |
| | | | | | AGTCCTCTGGTCTATCTCATGAAAC | (SEQ ID NO: 4449) |
| 202806_at | DBN1 | D0S1173, | drebrin 1 | 1627 | ATAGGAGACTTGTACAGTTGACTGG | (SEQ ID NO: 4450) |
| | | DKFZp434D064 | | | GGCTTTCCTCTCGTTGGTAGTTGAG | (SEQ ID NO: 4451) |
| | | | | | TGAGACGCTGTTGCAAATTCCACCC | (SEQ ID NO: 4452) |
| | | | | | TCCTTCCCTGGTCCAGATTGTAGCT | (SEQ ID NO: 4453) |
| | | | | | TCCAGATTGTAGCTCTTAGTCCTCC | (SEQ ID NO: 4454) |
| | | | | | TCAGCTGGCCGGGTTGGAGGCCTCA | (SEQ ID NO: 4455) |
| | | | | | GCTCTGGTGGGAAAATGTCCCCCAC | (SEQ ID NO: 4456) |
| | | | | | GTTTCTTGGGAAAATATCACTTTGT | (SEQ ID NO: 4457) |
| | | | | | ATATCACTTTGTATTCTCTGTCCAG | (SEQ ID NO: 4458) |
| | | | | | TTTGTATTCTCTGTCCAGGGCTTCA | (SEQ ID NO: 4459) |
| | | | | | CCAGGGCTTCAGATATTTTGCACGA | (SEQ ID NO: 4460) |
| 202875_s_{—} | PBX2 | DAAP- | pre-B-cell | 5089 | CTCCCTAACGAGACTCTCTGTGCTG | (SEQ ID NO: 4461) |
| at | | 218M18.5, | leukemia | | GTGCTGGGGGTGCTAATTACATGGC | (SEQ ID NO: 4462) |
| | | G17, HOX12, | homeobox 2 | | GAATGGGGCCTCTAAGGGGAGTGTG | (SEQ ID NO: 4463) |
| | | PBX2MHC | | | GAGAAACCTATTTCTCAGACCCCTT | (SEQ ID NO: 4464) |
| | | | | | ACCCCACTTGTATGCTCTGGAATCT | (SEQ ID NO: 4465) |
| | | | | | ATGGGGACATGACTTCTGGACAGAG | (SEQ ID NO: 4466) |
| | | | | | TACCTCATACGCAGCTCATCTTAAA | (SEQ ID NO: 4467) |
| | | | | | CTCGGTGGACGAGAGTGTCTGACTC | (SEQ ID NO: 4468) |
| | | | | | GTCTGACTCAGATATCTACCTCGGA | (SEQ ID NO: 4469) |
| | | | | | ACCTCGGAGGGAGTTTCTGCTACTT | (SEQ ID NO: 4470) |
| | | | | | AGGGAATTATTGACTGGGCTTTGGG | (SEQ ID NO: 4471) |
| 203210_s_{—} | RFC5 | MGC1155, | replication | 5985 | ATGCGTAGGGCTCTGAACATTTTGC | (SEQ ID NO: 4472) |
| at | | RFC36 | factor C | | CAGAGGAGACTGTCTACACCTGCAC | (SEQ ID NO: 4473) |
| | | | (actibator 1) | | CACCCGCTCAAGTCAGACATTGCCA | (SEQ ID NO: 4474) |
| | | | 5, 36.5 kDa | | ATAGAGTTGACTTTCCATCTTCAGT | (SEQ ID NO: 4475) |
| | | | | | GAGTACAGGCTTTCTGTTGGCACCA | (SEQ ID NO: 4476) |
| | | | | | ACGAGAAGATCCAGCTGAGCTCCCT | (SEQ ID NO: 4477) |
| | | | | | GCTGCATTTCAAGTCACCAGAGACC | (SEQ ID NO: 4478) |
| | | | | | GAGACCTGATTGTTGCAGAGGCCTA | (SEQ ID NO: 4479) |
| | | | | | GCCTAGATGCTCTGAGGGCCATTCA | (SEQ ID NO: 4480) |
| | | | | | GGGCCATTCACAATTCTCAGGGCTC | (SEQ ID NO: 4481) |
| | | | | | ATCAGTCACCCCGAATCTTGGAAAA | (SEQ ID NO: 4482) |
| 203327_at | IDE | RP11- | insulin- | 3416 | TCATCACTTCCTTCTGGTTTTATGT | (SEQ ID NO: 4483) |
| | | 366I13.1, | degrading | | TTCCGATCCACCACTGTACTAGTAA | (SEQ ID NO: 4484) |
| | | FLJ35968, | enzyme | | GTCTTCCATGAACCTGCAATTGTTT | (SEQ ID NO: 4485) |
| | | INSULYSIN | | | TATGCGTCAGCATTTTCCAATTTCC | (SEQ ID NO: 4486) |
| | | | | | AGGTTGGATCTAGAGCTGCTGTTGA | (SEQ ID NO: 4487) |
| | | | | | GCTGCTGTTGATCACTCAGGCATAC | (SEQ ID NO: 4488) |
| | | | | | GTCCAAGCTGCAGTCCATGAGCAAT | (SEQ ID NO: 4489) |
| | | | | | CATGAGCAATAACAGACTACCCCAG | (SEQ ID NO: 4490) |
| | | | | | GACTACCCCAGATACTGCAGTTTAC | (SEQ ID NO: 4491) |
| | | | | | GAAAGTCTTCTTTTTTGACCAGTTG | (SEQ ID NO: 4492) |
| | | | | | GAGGGAGCATGTGACACAGCCAGTA | (SEQ ID NO: 4493) |
| 203362_s_{—} | MAD2L1 | HSMAD2, | MAD2 mitotic | 4085 | AAATGATACTTACTGAACTGTGTGT | (SEQ ID NO: 4494) |
| at | | MAD2 | arrest | | GTACCTATTTGACTTACCATGGAGT | (SEQ ID NO: 4495) |
| | | | deficient- | | GGAGGTTTTTTTGTCAACATTGTGA | (SEQ ID NO: 4496) |
| | | | like | | AAGCTAGATGCTTTCCTAAATCAGA | (SEQ ID NO: 4497) |
| | | | 1 (yeast) | | CAGAATCTTTGTTAAGGTCCTGAAA | (SEQ ID NO: 4498) |
| | | | | | AGGTCCTGAAAGTAACTCATAATCT | (SEQ ID NO: 4499) |
| | | | | | ATTGCTGTATAGCTCCTTTTGACCT | (SEQ ID NO: 4500) |
| | | | | | CTCCTTTTGACCTTCATTTCATGTA | (SEQ ID NO: 4501) |
| | | | | | ATTTCATGTATAGTTTTCCCTATTG | (SEQ ID NO: 4502) |
| | | | | | GTTTTCCCTATTGAATCAGTTTCCA | (SEQ ID NO: 4503) |
| | | | | | ATTTGTACTGTTTAATGTTCTGTGA | (SEQ ID NO: 4504) |
| 203396_at | PSMA4 | HC9, | proteasome | 5685 | GGCTTTCAGCTCTATCAGAGTGACC | (SEQ ID NO: 4505) |
| | | HsT17706, | (prosome, | | AGAGTGACCCTAGTGGAAATTACGG | (SEQ ID NO: 4506) |
| | | MGC111191, | macropain) | | GGGGGATGGAAGGCCACATGCATTG | (SEQ ID NO: 4507) |
| | | MGC12467, | subunit, | | GAAATAATAGCGCTGCAGCTGTGTC | (SEQ ID NO: 4508) |
| | | MGC24813, | alpha type, 4 | | GCGCTGCAGCTGTGTCAATGTTGAA | (SEQ ID NO: 4509) |
| | | PSC9 | | | GAAATGACCTTGAAGTCAGCACTTG | (SEQ ID NO: 4510) |
| | | | | | CATGGATGTTAGTAAACTCTCTGCT | (SEQ ID NO: 4511) |
| | | | | | AAGACAGTAATCAGAGTTCTCAAAC | (SEQ ID NO: 4512) |
| | | | | | TACTGAATTGGGTCCTTGTCATTTC | (SEQ ID NO: 4513) |
| | | | | | GTCCTTGTCATTTCTGTCCAATTGA | (SEQ ID NO: 4514) |
| | | | | | TACCCTTCATGGACGTCTTAATCTT | (SEQ ID NO: 4515) |
| 203478_at | NDUFC1 | KFYI, | NADH | 4717 | GGGGCCCAAGGAATTCGCAAGATGG | (SEQ ID NO: 4516) |
| | | MGC117464, | dehydrogenase | | AGGAATTCGCAAGATGGCGCCGTCC | (SEQ ID NO: 4517) |
| | | MGC126847, | (ubiquinone) | | GCCCTTCAGTGCGATCAAAGTTCTA | (SEQ ID NO: 4518) |
| | | MGC138266 | 1, | | GTGCGATCAAAGTTCTACGTGCGAG | (SEQ ID NO: 4519) |
| | | | subcomplex | | TTCTACGTGCGAGAGCCGCCGAATG | (SEQ ID NO: 4520) |
| | | | unknow, 1, | | CCGCCGAATGCCAAACCTGACTGGC | (SEQ ID NO: 4521) |
| | | | 6 kDa | | ACCTGACTGGCTGAAAGTTGGGTTC | (SEQ ID NO: 4522) |
| | | | | | TGAAAGTTGGGTTCACCTTGGGCAC | (SEQ ID NO: 4523) |
| | | | | | GTCTTCTTGTGGATCTATCTCATCA | (SEQ ID NO: 4524) |
| | | | | | GGATCTATCTCATCAAACAACACAA | (SEQ ID NO: 4525) |
| | | | | | CAAGCCAGCATTTGTATTTTGCATC | (SEQ ID NO: 4526) |
| 203682_s_{—} | IVD | ACAD2 | isovaleryl | 3712 | GAAGTAGAGATGTGGCGGCTTTCCC | (SEQ ID NO: 4527) |
| at | | | Coenzyme A | | GAGGTTGAGTTCTCCACAACAGCTC | (SEQ ID NO: 4528) |
| | | | dehydrogenase | | AACAGCTCCCAAGCATCATGGGCCT | (SEQ ID NO: 4529) |
| | | | | | CTGTGCCACGGCTAGTGTTGTGTGA | (SEQ ID NO: 4530) |
| | | | | | CACCTCCCAGGGTAGGCACCTGGGG | (SEQ ID NO: 4531) |
| | | | | | CCACCTCCCAGGGTAGGCACCTGGG | (SEQ ID NO: 4532) |
| | | | | | ACCTCTTTCTCTTGGGTGAGGCTCT | (SEQ ID NO: 4533) |
| | | | | | GAGGCTCTGGCAAGGAGATCTCTCT | (SEQ ID NO: 4534) |
| | | | | | AGATCTCTCTGCTCAAGCACAGCAG | (SEQ ID NO: 4535) |
| | | | | | GGCCCCTCTCCATGAATTGGAACTT | (SEQ ID NO: 4536) |
| | | | | | TCAACTTTCAGTCTCTTTTCTGGGG | (SEQ ID NO: 4537) |
| 204228_at | PPIH | KFYI, | peptidylprolyl | 10465 | ATGGAGATGGTACTGGAGTCGCCAG | (SEQ ID NO: 4538) |
| | | MGC117464, | isomerase H | | TCGCCAGTATTTACCGGGGGCCATT | (SEQ ID NO: 4539) |
| | | MGC126847, | (cyclophilin | | TTTAAACTTAGACACTCAGCTCCAG | (SEQ ID NO: 4540) |
| | | MGC138266 | H) | | GCTTTCCATGGCGAACAGTGGTCCA | (SEQ ID NO: 4541) |
| | | | | | ATCACCTGCTCTAAGTGCGATTGGC | (SEQ ID NO: 4542) |
| | | | | | AAAATCATCGATGGACTTCTAGTGA | (SEQ ID NO: 4543) |
| | | | | | TTCCCACAGGCCCCAACAATAAGCC | (SEQ ID NO: 4544) |
| | | | | | CTACCTGTGGTGATCTCGCAGTGTG | (SEQ ID NO: 4545) |
| | | | | | AAAGACTGAATCAGGCCTTCCCTTC | (SEQ ID NO: 4546) |
| | | | | | AGTAAGATAATCTGGACTGGCCCCC | (SEQ ID NO: 4547) |
| | | | | | CTGCTGCCCCATTTGATCAAGAGAC | (SEQ ID NO: 4548) |
| 204321_at | NEO1 | DKFZp547A066, | neogenine | 4756 | GAAGCCTGTGTCGAGGCAGCTTCCC | (SEQ ID NO: 4549) |
| | | DKFZp547B146, | homolog 1 | | TCCCTTTGCCTGCTGATATTCTGCA | (SEQ ID NO: 4550) |
| | | HsT17534, | (chicken) | | GCAACCTGCATTTCACTTTGTGGTC | (SEQ ID NO: 4551) |
| | | NGN | | | TTTCACTTTGTGGTCAGGCCGTGTC | (SEQ ID NO: 4552) |
| | | | | | CGTGTCTTTGTGCTGTGACTGCATC | (SEQ ID NO: 4553) |
| | | | | | GTGACTGCATCACCTTTATGGAGTG | (SEQ ID NO: 4554) |
| | | | | | GTCCTTGGTGTTCTCCACAAGTGGT | (SEQ ID NO: 4555) |
| | | | | | GGTTGACATTTGACTGCTTGTTCCA | (SEQ ID NO: 4556) |
| | | | | | TTTGACAGTGTGGGTCGTTCCTGGG | (SEQ ID NO: 4557) |
| | | | | | TTCTGAGTCATTGCATCCTCTACCA | (SEQ ID NO: 4558) |
| | | | | | TCTACCAGCTGTTAATCCATCACTC | (SEQ ID NO: 4559) |
| 204533_at | CXCL10 | C7, IFI10, | chemokine | 3627 | TAACTCTACCCTGGCACTATAATGT | (SEQ ID NO: 4560) |
| | | INP10, IP-10, | (C-X-C motif) | | GCTCTACTGAGGTGCTATGTTCTTA | (SEQ ID NO: 4561) |
| | | SCYB10, crg- | ligand 10 | | TCTTAGTGGATGTTCTGACCCTGCT | (SEQ ID NO: 4562) |
| | | 2, gIP-10, | | | TCTGACCCTGCTTCAAATATTTCCC | (SEQ ID NO: 4563) |
| | | mob-1 | | | GGTACTAAGGAATCTTTCTGCTTTG | (SEQ ID NO: 4564) |
| | | | | | GCTTTGGGGTTTATCAGAATTCTCA | (SEQ ID NO: 4565) |
| | | | | | AATGCTCTTTACTTCATGGACTTCC | (SEQ ID NO: 4566) |
| | | | | | TTACTTCATGGACTTCCACTGCCAT | (SEQ ID NO: 4567) |
| | | | | | ATTCTTTCAGTGGCTACCTACATAC | (SEQ ID NO: 4568) |
| | | | | | GCTACCTACATACAATTCCAAACAC | (SEQ ID NO: 4569) |
| | | | | | AATGAGGTACTCTCCTGGAAATATT | (SEQ ID NO: 4570) |
| 205565_s_{—} | FXN | CyaY, FA, | frataxin | 2395 | AACTGTCAAACTGGGTGGAGATCTA | (SEQ ID NO: 4571) |
| at | | FARR, FRDA, | | | AGGAACCTATGTGATCAACAAGCAG | (SEQ ID NO: 4572) |
| | | MGC57199, | | | CAAGCAGACGCCAAACAAGCAAATC | (SEQ ID NO: 4573) |
| | | X25 | | | TTCTCCATCCAGTGGACCTAAGCGT | (SEQ ID NO: 4574) |
| | | | | | ATCCAGTGGACCTAAGCGTTATGAC | (SEQ ID NO: 4575) |
| | | | | | TTGGCCTATTCCGGAAAAGATGCTT | (SEQ ID NO: 4576) |
| | | | | | CGGAAAAGATGCTTGATGCCCAGCC | (SEQ ID NO: 4577) |
| | | | | | TAAGGACATTAAAAGCTATCAGGCC | (SEQ ID NO: 4578) |
| | | | | | TTGGGCTATGTGTCACAGCTCTGTA | (SEQ ID NO: 4579) |
| | | | | | TTTTGGATTGTCGGATTTCCTCCCT | (SEQ ID NO: 4580) |
| | | | | | TAATGTCTTATGCCTATACCTGAAT | (SEQ ID NO: 4581) |
| 205890_s_{—} | GABBR1/// | DAAP- | gamma- | 10537 | GATCTTAAAGCCACGGAGAAGCCTC | (SEQ ID NO: 4582) |
| at | UBD | 188P13.3, | aminobutyric | 2550 | TGCCCTTGTTTCTTGTGGAGTCAGG | (SEQ ID NO: 4583) |
| | | FLJ92613, | acid (GABA) | | ATGAGGCAAAGAGGCACCTCCTCCA | (SEQ ID NO: 4584) |
| | | GABAB(1e), | B receptor, | | GAAGGTCCAGCTCAGTGGCACAAGT | (SEQ ID NO: 4585) |
| | | GABABR1, | 1/// | | GACGGGTATAATCCCTGAGACCCAG | (SEQ ID NO: 4586) |
| | | GABBR1-3, | ubiquitin D | | GACCCAGATTGTGACTTGCAATGGA | (SEQ ID NO: 4587) |
| | | GPRC3A, | | | AGAAAGGGCAACTTACTCTTCCTGG | (SEQ ID NO: 4588) |
| | | dJ271M21.1.1, | | | ACTCTTCCTGGCATCTTATTGTATT | (SEQ ID NO: 4589) |
| | | dJ271M21.1.2, | | | AATCTCTTACTCAACGAACACATCT | (SEQ ID NO: 4590) |
| | | hGF1a/// | | | CATCTTCTGATGATTTCCCAAAATT | (SEQ ID NO: 4591) |
| | | DAMC- | | | ATTGCCCAACTCTATGTTTCTTTGA | (SEQ ID NO: 4592) |
| | | 346D16.1, | | | | |
| | | FAT10, | | | | |
| | | GABBR1, | | | | |
| | | UBD-3 | | | | |
| 205980_s_{—} | ARHGAP8/// | BPGAP1, | Rho GTPase | 23779 | GCCTGTGTCTTCGGGCTGAATTTGA | (SEQ ID NO: 4593) |
| at | LOC553158 | FLJ20185, | activating | 553158 | GTGTCTTCGGGCTGAATTTGATCTG | (SEQ ID NO: 4594) |
| | ///PRR5/// | PP610/// | protein 8/// | 55615 | TGAATTTGATCTGGCCATCCCAGGG | (SEQ ID NO: 4595) |
| | ARHGAP8 | LOC553158; | proline rich | | AGTCCCCTGATGGCAGCCAGAAGAC | (SEQ ID NO: 4596) |
| | | ARHGAP8/// | 5 (renal)/// | | TGATGGCAGCCAGAAGACGTCTCTA | (SEQ ID NO: 4597) |
| | | | PRR5- | | GGCAGCCAGAAGACGTCTCTAGTGT | (SEQ ID NO: 4598) |
| | | | ARHGAP8 | | GCCAGAAGACGTCTCTAGTGTTGCG | (SEQ ID NO: 4599) |
| | | | fusion | | GAAGACGTCTCTAGTGTTGCGAACA | (SEQ ID NO: 4600) |
| | | | | | GTCTCTAGTGTTGCGAACACTCTGT | (SEQ ID NO: 4601 |
| | | | | | TGTGCACTTGTATGTTTTGTAAACT | (SEQ ID NO: 4602) |
| | | | | | TTGTAAACTTGGCATCTGTAAAAAT | (SEQ ID NO: 4603) |
| 206289_at | HOXA4 | HOXA-4 | homeobox | 3201 | CCATCTGGACCATAATAGGGACACC | (SEQ ID NO: 4604) |
| | | | A4 | | CGGTTAAGAGTCTGTGCTGGTTGCC | (SEQ ID NO: 4605) |
| | | | | | GAGAACAGTTGTCCAGGATGCTGGC | (SEQ ID NO: 4606) |
| | | | | | ACAACCTGCTGGCCCGAAACAAGGC | (SEQ ID NO: 4607) |
| | | | | | AACAAGGCTGCCAGGTGTGGAACAG | (SEQ ID NO: 4608) |
| | | | | | AACAGCCCATGCTGACTGGGGACAT | (SEQ ID NO: 4609) |
| | | | | | GACTGGGGACATACACTTGCATCTT | (SEQ ID NO: 4610) |
| | | | | | CCCCATTAAGGCAGCTCATCCAAGC | (SEQ ID NO: 4611) |
| | | | | | GACATGTTAGTTTTTCTTACTTGCA | (SEQ ID NO: 4612) |
| | | | | | CAGAGATACTGTTGTCACCTTCTGG | (SEQ ID NO: 4613) |
| | | | | | GGGGAGTGCATTTAGACTTATGTGG | (SEQ ID NO: 4614) |
| 206441_s_{—} | COMMD4 | FLJ20452 | COMM | 54939 | GGAGCTGCTGGGACAGGGGATTGAT | (SEQ ID NO: 4615) |
| at | | | domain | | AGCTCACGGCTGACGCCAAGTTTGA | (SEQ ID NO: 4616) |
| | | | containing 4 | | TCCAGTGCGGCCAAGCACAGTGTCG | (SEQ ID NO: 4617) |
| | | | | | AGTGTCGATGGCGAATCCTTGTCCA | (SEQ ID NO: 4618) |
| | | | | | TCCTTGTCCAGTGAACTGCAGCAGC | (SEQ ID NO: 4619) |
| | | | | | AGCTGGGGCTGCCCAAAGAGCACGC | (SEQ ID NO: 4620) |
| | | | | | CCAGCCTGTGCCGCTGTTATGAGGA | (SEQ ID NO: 4621) |
| | | | | | CAAAGCCCCTTGCAGAAGCACTTGC | (SEQ ID NO: 4622) |
| | | | | | TGAACTGCTCTTCGGGAGGCAGCCC | (SEQ ID NO: 4623) |
| | | | | | GCCCTGGTTCTAGGATGCTGAGGCC | (SEQ ID NO: 4624) |
| | | | | | GCCTCACTTCTCTCTTGAGAACTTG | (SEQ ID NO: 4625) |
| 206752_s_{—} | DFFB | RP13- | DNA | 1677 | TGAACAGCTATCACGTTGAACCACG | (SEQ ID NO: 4626) |
| at | | 531C17.1, | fragmentation | | GAAACTGCTGTTTTCTAGGCCAAAA | (SEQ ID NO: 4627) |
| | | CAD, CPAN, | factor, | | TGTGTCACAGTTTCAGGCTTTTATG | (SEQ ID NO: 4628) |
| | | DFF-40, DFF2 | 40 kDa, beta | | TGGAAGCTGTTCAGGGCATCGGCAG | (SEQ ID NO: 4629) |
| | | DFF40 | polypeptide | | GGTCAGCGTTACTATCATCTCGGAT | (SEQ ID NO: 4630) |
| | | | (caspase- | | GGATCATATGGAGCTCATGTCAGCC | (SEQ ID NO: 4631) |
| | | | activated | | TCATGTCAGCCGTGTGGGTGGCGGG | (SEQ ID NO: 4632) |
| | | | DNase) | | GCGGGTGCACAGAGACGGTCTGGAA | (SEQ ID NO: 4633) |
| | | | | | GGAAGGAAACACGCGGATCTGAACA | (SEQ ID NO: 4634) |
| | | | | | GGTTGGGCTAGATTACAGAGGGCTC | (SEQ ID NO: 4635) |
| | | | | | TACAGAGGGCTCATTTTCTACGTCA | (SEQ ID NO: 4636) |
| 206966_s_{—} | KLF12 | AP-2rep, | Kruppel-like | 11278 | CCGCCTTCAAGTCCCATGAATTTAC | (SEQ ID NO: 4637) |
| at | | AP2REP, | factor 12 | | CAAACTGAGTCATGTTCACCGCATC | (SEQ ID NO: 4638) |
| | | HSPC122 | | | CATCCCCGTGGTGGTACAGTCGGTG | (SEQ ID NO: 4639) |
| | | | | | TGGTACAGTCGGTGCCTGTTGTCTA | (SEQ ID NO: 4640) |
| | | | | | GTCTACACAGCTGTAAGGTCACCTG | (SEQ ID NO: 4641) |
| | | | | | GAACAACACTATTGTCGTGCCGCTT | (SEQ ID NO: 4642) |
| | | | | | GTCGTGCCGCTTTTGGAGGATGGGA | (SEQ ID NO: 4643) |
| | | | | | TGGCAAAGCACAAATGGACCCCCGA | (SEQ ID NO: 4644) |
| | | | | | AGGCCTATCTCCCAGACAAAGTAAA | (SEQ ID NO: 4645) |
| | | | | | AAATGTGACCTTAGATAGCGTTAAT | (SEQ ID NO: 4646) |
| | | | | | CCCTTTCCATAGCCAGAGCAGTACA | (SEQ ID NO: 4647) |
| 207153_s_{—} | GLMN | FAP, FAP48, | glomulin, | 11146 | GAGTTTAGCTATGCTTCAGCTGTAT | (SEQ ID NO: 4648) |
| at | | FAP68, | FKBP | | TACAGGACCACAGTTGATTTCCCTT | (SEQ ID NO: 4649) |
| | | FKBPAP, | associated | | GATTTCCCTTCTTGATTTGGTACTT | (SEQ ID NO: 4650) |
| | | GLML, GVM, | protein | | CTCCCAGAGGGTGCAGAAACAGATT | (SEQ ID NO: 4651) |
| | | VMGLOM | | | AAAGCCACTTCATATAGGACTTAAT | (SEQ ID NO: 4652) |
| | | | | | TAGCCAAGAGGCCCAGAAATCTAAA | (SEQ ID NO: 4653) |
| | | | | | GAAGAGATCCCTAATATGCCTCCTG | (SEQ ID NO: 4654) |
| | | | | | AATATGCCTCCTGAAATGCAGCTTA | (SEQ ID NO: 4655) |
| | | | | | TTAAGGTCCTGCATTCAGCTCTTTT | (SEQ ID NO: 4656) |
| | | | | | TTGAAAGTGTTCTAGCTCGAGTGGA | (SEQ ID NO: 4657) |
| | | | | | ATAAAAACAAAGTCTACCTCTGAAG | (SEQ ID NO: 4658) |
| 207181_s_{—} | CASP7 | CMH-1, ICE- | caspase 7, | 840 | GGGAGTTAGGACCACCAATGGATTG | (SEQ ID NO: 4659) |
| at | | LAP3, MCH3 | apoptosis- | | GAGATGGAGTGAGCTGAGACAGATA | (SEQ ID NO: 4660) |
| | | | related | | CTTACTGCCCAGGCCTATCTGGAAG | (SEQ ID NO: 4661) |
| | | | cysteine | | GAAATGGATGTAAGCCTGGCCCATA | (SEQ ID NO: 4662) |
| | | | peptidase | | AAGCCTGGCCCATAATGTGAACATA | (SEQ ID NO: 4663) |
| | | | | | AAGTAATCACTAATGCTCAACAATT | (SEQ ID NO: 4664) |
| | | | | | GCTCAACAATTTATCCATTCAATCA | (SEQ ID NO: 4665) |
| | | | | | TTCATTGGGTTGTCAGATAGTCTAT | (SEQ ID NO: 4666) |
| | | | | | TTTAGATTATTCCAAGCCTTATTTT | (SEQ ID NO: 4667) |
| | | | | | GATTTTTGGCACTTTGTTTTCAAGA | (SEQ ID NO: 4668) |
| | | | | | TTTTCTGCCTAATAGTAACTGGTTA | (SEQ ID NO: 4669) |
| 207469_s_{—} | PIR | 0 | pirin (iron- | 8544 | ACTCGCACACCAACCTTATATTTGG | (SEQ ID NO: 4670) |
| at | | | binding | | GGACTTCAAATTGGACCCAGGAGCC | (SEQ ID NO: 4671) |
| | | | nuclear | | GGACAAGCTTCATTTACACGATATC | (SEQ ID NO: 4672) |
| | | | protein) | | GGAGATGTGTATATTGGGCCCGATG | (SEQ ID NO: 4673) |
| | | | | | ATTGGGCCCGATGATGCACAACAAA | (SEQ ID NO: 4674) |
| | | | | | CTCATCACACAGCAGTGCTTGGAGA | (SEQ ID NO: 4675) |
| | | | | | GAAGCCACTTTGTCTTAATTGCTGG | (SEQ ID NO: 4676) |
| | | | | | GAGAACCAGTTATCCAACATGGTCC | (SEQ ID NO: 4677) |
| | | | | | CGGAAGAGCAGGTCTTGATGTGTCC | (SEQ ID NO: 4678) |
| | | | | | GAAGGCATTCCATTTCTAAAGCTTA | (SEQ ID NO: 4679) |
| | | | | | GCTTCTAAAGAATTCCACACTAACG | (SEQ ID NO: 4680) |
| 207507_s_{—} | ATP5G3 | MGC125738, | ATP | 518 | CGCGCAGTCCCGAAGAGTGTAAGAT | (SEQ ID NO: 4681) |
| at | | P3 | synthase, H+ | | CCTCTCTGATCCGAGCTGGATCCAG | (SEQ ID NO: 4682) |
| | | | transporting, | | GCATACAGACCAATTTCTGCATCAG | (SEQ ID NO: 4683) |
| | | | mitochondrial | | TGCATCAGTGTTATCTCGACCAGAG | (SEQ ID NO: 4684) |
| | | | F0 complex, | | ATCTCGACCAGAGGCTAGTAGGACT | (SEQ ID NO: 4685) |
| | | | subunit C3 | | TGGGGCCCAGAATGGTGTGTCTCAG | (SEQ ID NO: 4686) |
| | | | (subunit 9) | | GTGTCTCAGCTAATCCAAAGGGAGT | (SEQ ID NO: 4687) |
| | | | | | TTGTTTGATGGTTGCTTTCTTGATT | (SEQ ID NO: 4688) |
| | | | | | ACTGCTTGACATGTTGGCATTCATA | (SEQ ID NO: 4689) |
| | | | | | TGTGTATCTTACTGTGACTCCGAAA | (SEQ ID NO: 4690) |
| | | | | | AAAACTGTAGTATTGGTGTCATGGG | (SEQ ID NO: 4691) |
| 208636_at | ACTN1 | FLJ40884 | actinin, alpha | 87 | AACTATTTGCACCGAAATGTCTTGT | (SEQ ID NO: 4692) |
| | | | 1 | | GTCTTGTTTTGTTGCGACATAGGAA | (SEQ ID NO: 4693) |
| | | | | | CAAGCACAAAGTTATATTCCATCCT | (SEQ ID NO: 4694) |
| | | | | | ATATTCCATCCTTTTTACTGATTTT | (SEQ ID NO: 4695) |
| | | | | | CAATCTCATGTCCATTTTGGTGTGG | (SEQ ID NO: 4696) |
| | | | | | CATTTTGGTGTGGGAGTCGGGGTAG | (SEQ ID NO: 4697) |
| | | | | | ATGTGTGTTTGCTAGCTCACTTGTC | (SEQ ID NO: 4698) |
| | | | | | TCTCATTTTTAAATCCCCTCAGAGG | (SEQ ID NO: 4699) |
| | | | | | AAATCCCCTCAGAGGTGTGACTAGT | (SEQ ID NO: 4700) |
| | | | | | GACTAGTCTCTTTATCAGCACACAC | (SEQ ID NO: 4701) |
| | | | | | GGACAACTTTGATATTGCTTGGCAC | (SEQ ID NO: 4702) |
| 208675_s_{—} | DDOST | AGE-R1, | dolichyl- | 1650 | CATCGTGATCCAGCAGCTCTCAAAT | (SEQ ID NO: 4703) |
| at | | KIAA0115, | diphospho- | | CTCAAATGGCAAATGGGTCCCCTTT | (SEQ ID NO: 4704) |
| | | MGC2191, | oligo- | | GGTCCCCTTTGATGGCGATGACATT | (SEQ ID NO: 4705) |
| | | OK/SW-cl.45, | saccharide- | | GGAGTTTGTCCGCATTGATCCTTTT | (SEQ ID NO: 4706) |
| | | OST, OST48, | protein | | GATCCTTTTGTGAGGACCTTCCTGA | (SEQ ID NO: 4707) |
| | | WBP1 | glycosyl- | | ACAGTGTTCAGTTCAAGTTGCCCGA | (SEQ ID NO: 4708) |
| | | | transferase | | GTTGCCCGACGTGTATGGTGTATTC | (SEQ ID NO: 4709) |
| | | | | | GATTACAACCGGCTAGGCTACACAC | (SEQ ID NO: 4710) |
| | | | | | CCTGTACTCTTCCACTCAGGTATCC | (SEQ ID NO: 4711) |
| | | | | | CACACGCAGTATGAGCGCTTCATCC | (SEQ ID NO: 4712) |
| | | | | | ATGGCACAACTTTACCTCTGTGGGA | (SEQ ID NO: 4713) |
| 208829_at | TAPBP | DAMC- | TAP binding | 6892 | TTCTTCAGCCCTAGATGGTGCTCGC | (SEQ ID NO: 4714) |
| | | 227D19.13, | protein | | CCTCTCAATGCTCATCACACACAGG | (SEQ ID NO: 4715) |
| | | NGS17, TAPA, | (tapasin) | | TCACACACAGGGCTATTCCTTTCCT | (SEQ ID NO: 4716) |
| | | TPN, TPSN, | | | TCCTTTCCTCCAATGAACCAAACCG | (SEQ ID NO: 4717) |
| | | tapasin | | | AGACGAGGTCGGCCTCGTCATTCCC | (SEQ ID NO: 4718) |
| | | | | | CTCTTGTGCGGTTCACCACAGTTGT | (SEQ ID NO: 4719) |
| | | | | | TAAGTGATCGTGTGAGTCGTCGTTA | (SEQ ID NO: 4720) |
| | | | | | GAGTCGTCGTTAAATGCCTGTCTCC | (SEQ ID NO: 4721) |
| | | | | | GCATAGGGACCTAAGGCCCACTGGA | (SEQ ID NO: 4722) |
| | | | | | TGGAGGGCGCTCATCAAGTAGCTGC | (SEQ ID NO: 4723) |
| | | | | | GATCTCCGAGTCAGGACGGTCGGCC | (SEQ ID NO: 4724) |
| 209110_s_{—} | RGL2 | DADB- | ral guanine | 5863 | GCCTCAGGAACTCCTCCGAGTGAGG | (SEQ ID NO: 4725) |
| at | | 159G18.6, | nucleotide | | CTCCTTTCCCAGGATCAAGGCCACA | (SEQ ID NO: 4726) |
| | | HKE1.5, | dissociation | | GAAGATTGCACGGGCACTGTTCTGA | (SEQ ID NO: 4727) |
| | | KE1.5, RAB2L | stimulator- | | ACTGTTCTGAGGAGGAAGCCCCGTT | (SEQ ID NO: 4728) |
| | | | like 2 | | AGCCCCGTTGGCTTACAGAAGTCAT | (SEQ ID NO: 4729) |
| | | | | | GTTGGATACCTGTGTATAGCTTCCC | (SEQ ID NO: 4730) |
| | | | | | GATTCTTGGCCATATCCTAACATGC | (SEQ ID NO: 4731) |
| | | | | | CAAAGGCTTCAAGGCTCTGAGCCCC | (SEQ ID NO: 4732) |
| | | | | | AATGTAGGTCCTCGCAGGAGCTCTT | (SEQ ID NO: 4733) |
| | | | | | TTCTTCCCACTCTGGGGGTTTCTAT | (SEQ ID NO: 4734) |
| | | | | | TGGGGGTTTCTATCACTGTGACAAC | (SEQ ID NO: 4735) |
| 209164_s_{—} | CYB561 | FRRS2 | cytochrome | 1534 | TTAAATGAAAAACCGCTGCTGCTGG | (SEQ ID NO: 4736) |
| at | | | b-561 | | CTGTTCTGGAGGACGAGCCTTCTCC | (SEQ ID NO: 4737) |
| | | | | | TTCTGGAGGACGAGCCTTCTCCTTA | (SEQ ID NO: 4738) |
| | | | | | CTGCCCATCTTTCCAGGAAGTCAGG | (SEQ ID NO: 4739) |
| | | | | | CTCCAGCCCCATAAAGAGTGTCATG | (SEQ ID NO: 4740) |
| | | | | | AGAGTGTCATGTTAGCTGAGTCACC | (SEQ ID NO: 4741) |
| | | | | | GTGTCATGTTAGCTGAGTCACCATT | (SEQ ID NO: 4742) |
| | | | | | TAGCTGAGTCACCATTTGGCTTCGG | (SEQ ID NO: 4743) |
| | | | | | TCACCATTTGGCTTCGGCCTGGAAA | (SEQ ID NO: 4744) |
| | | | | | CATTTGGCTTCGGCCTGGAAATAGT | (SEQ ID NO: 4745) |
| | | | | | GAACACTGATCGTGTGCGAGGCCAG | (SEQ ID NO: 4746) |
| 209228_x_{—} | TUSC3 | D8S1992, | tumor | 7991 | ACCCAACTACTCTGGTACCATTGCT | (SEQ ID NO: 4747) |
| at | | M33, | suppressor | | AGACTGGTTGGGCCATGGTGTCTCT | (SEQ ID NO: 4748) |
| | | MGC13453, | candidate 3 | | GGTGTCTCTGTGTATAGTCTTTGCT | (SEQ ID NO: 4749) |
| | | MRT7, N33, | | | CTTTGCTATGACTTCTGGCCAGATG | (SEQ ID NO: 4750) |
| | | OST3A | | | ATGTGGAACCATATCCGTGGACCTC | (SEQ ID NO: 4751) |
| | | | | | GGAGCAGCCAGGCTCAGTTTGTGGC | (SEQ ID NO: 4752) |
| | | | | | TCTGGTACTGAATGCCGCTATCACC | (SEQ ID NO: 4753) |
| | | | | | CCTAGTGGGATTGGGCCTGGTGGTC | (SEQ ID NO: 4754) |
| | | | | | CTTCTTCTTCAGTTTTCTACTTTCA | (SEQ ID NO: 4755) |
| | | | | | CCAAGTACCACGGCTATCCTTATAG | (SEQ ID NO: 4756) |
| | | | | | AACTCTATAACCTCAGCCTTTTAAT | (SEQ ID NO: 4757) |
| 209285_s_{—} | C3orf63 | DKFZp686C2456, | chromosome | 23272 | GAGGGTATTGGTACATGACGACATG | (SEQ ID NO: 4758) |
| at | | KIAA1105, | 3 open | | TTTAAACACAGCTTACTCTTCCTCA | (SEQ ID NO: 4759) |
| | | RAP140, se89-1 | reading | | ATCAAAACTAACTACAGCCATTCTT | (SEQ ID NO: 4760) |
| | | | frame 63 | | CATTCTTTTTAAAGCCCAAGGGATG | (SEQ ID NO: 4761) |
| | | | | | GGTGTAAGGATGTTACCTGTTTGTT | (SEQ ID NO: 4762) |
| | | | | | GTAGTATGGTGAGTAAGGTTATGCG | (SEQ ID NO: 4763) |
| | | | | | TTCTAACAAACACTGCCAACACGTC | (SEQ ID NO: 4764) |
| | | | | | AACACTGCCAACACGTCAATTTTTT | (SEQ ID NO: 4765) |
| | | | | | AAGCGTGGGCCACATTGCTAAGAAT | (SEQ ID NO: 4766) |
| | | | | | GCCTTATTGATGTTTTGCCGTTCCA | (SEQ ID NO: 4767) |
| | | | | | GTTTTGCCGTTCCAATGTATGCATT | (SEQ ID NO: 4768) |
| 209409_at | GRB10 | GRB-IR, Grb- | growth factor | 2887 | TGTATCTCTGCCTGTGATTTTCTTT | (SEQ ID NO: 4769) |
| | | 10, IRBP, | receptor- | | GAAACTCCATGTAGCAACCAGGACG | (SEQ ID NO: 4770) |
| | | KIAA0207, | bound | | TGTGCCTCAGCCTAGATGTTTTGTC | (SEQ ID NO: 4771) |
| | | MEG1, RSS | protein 10 | | TTCTCTTTTCTGCACTTAATACCTG | (SEQ ID NO: 4772) |
| | | | | | TGACAGTATGACCGATCTCTGCGCC | (SEQ ID NO: 4773) |
| | | | | | CGCCTTTCTGGGGGCGGGCAAGCTG | (SEQ ID NO: 4774) |
| | | | | | GCTGGCGGTAGATTTGTGATGTCAC | (SEQ ID NO: 4775) |
| | | | | | AACCTTTATTCCATGTGCTTTGCTT | (SEQ ID NO: 4776) |
| | | | | | CTTCATTCTGTACATAGCTCTTTGG | (SEQ ID NO: 4777) |
| | | | | | AGCTCTTTGGCTCGTGAACCTAATT | (SEQ ID NO: 4778) |
| | | | | | AGGGACTGATGTTCTGTTTCTTGTA | (SEQ ID NO: 4779) |
| 209506_s_{—} | NR2F1 | COUP-TFI, | nuclear | 7025 | CATCGTGCTGTTCACGTCAGACGCC | (SEQ ID NO: 4780) |
| at | | EAR-3, EAR3, | receptor | | GCAGTGCGCACTGGAGGAGTACGTG | (SEQ ID NO: 4781) |
| | | ERBAL3, | subfamily 2, | | AGCCGTTTTGGCAAACTGCTGCTGC | (SEQ ID NO: 4782) |
| | | NR2F2, | group F, | | GAAACTCTCATCCGCGATATGTTAC | (SEQ ID NO: 4783) |
| | | SVP44, | member1 | | TGTCTGGGAGCAGCTTCAACTGGCC | (SEQ ID NO: 4784) |
| | | TCFCOUP1, | | | CAACTGGCCTTACATGTCCATCCAG | (SEQ ID NO: 4785) |
| | | TFCOUP1 | | | CTAGAGACTCAGAGGACCCACCTGG | (SEQ ID NO: 4786) |
| | | | | | CGCGGGGACACCGGGAAGTGCAGCG | (SEQ ID NO: 4787) |
| | | | | | GAGGGCCGAGACAGGAGCAGCCCAC | (SEQ ID NO: 4788) |
| | | | | | GCCCACCCAGCAGAAATACAATCCG | (SEQ ID NO: 4789) |
| | | | | | GGATCAGATCTGTGAGCACGTTGGC | (SEQ ID NO: 4790) |
| 209592_s_{—} | WDR68 | AN11; HAN11 | WD repeat | 10238 | GATGGCTCGGTGCGGATGTTTGACC | (SEQ ID NO: 4791) |
| at | | | domain 68 | | ACAGCACCATCATTTACGAAGACCC | (SEQ ID NO: 4792) |
| | | | | | CATTTACGAAGACCCACAGCATCAC | (SEQ ID NO: 4793) |
| | | | | | TGCTTCGCCTCTGCTGGAACAAGCA | (SEQ ID NO: 4794) |
| | | | | | GAGATCAACAATGTGCAGTGGGCAT | (SEQ ID NO: 4795) |
| | | | | | CAGTGGGCATCAACTCAGCCCGACT | (SEQ ID NO: 4796) |
| | | | | | CCATCTGCTACAACAACTGCCTGGA | (SEQ ID NO: 4797) |
| | | | | | CTGCCTGGAGATACTCAGAGTGTAG | (SEQ ID NO: 4798) |
| | | | | | AGAGTGTAGTGTTGGTGGCGCTGTG | (SEQ ID NO: 4799) |
| | | | | | TGTGCCCACGAGGCAGGGGCTTTTG | (SEQ ID NO: 4800) |
| | | | | | GCAGGGGCTTTTGTATTTCCTGCCT | (SEQ ID NO: 4801) |
| 209774_x_{—} | CXCL2 | CINC-2a, | chemokine | 2920 | AGAGAGACACAGCTGCAGAGGCCAC | (SEQ ID NO: 4802) |
| at | | GROb, Gro2, | (C-X-C motif) | | ACCTGGATTGCGCCTAATGTGTTTG | (SEQ ID NO: 4803) |
| | | MIP-2, MIP-2a, | ligand 2 | | TGATTGAATCTACTTGCACACTCTC | (SEQ ID NO: 4804) |
| | | Mgsa-b, Mip2, | | | GCACACTCTCCCATTATATTTATTG | (SEQ ID NO: 4805) |
| | | Scyb, Scyb2 | | | AACCCAAGTTAGTTCAATCCTGATT | (SEQ ID NO: 4806) |
| | | | | | GAAGGTTTGCAGATATTCTCTAGTC | (SEQ ID NO: 4807) |
| | | | | | GACATATCACATGTCAGCCACTGTG | (SEQ ID NO: 4808) |
| | | | | | ATGGCCAGTAAGATCAATGTGACGG | (SEQ ID NO: 4809) |
| | | | | | TGTGACGGCAGGGAAATGTATGTGT | (SEQ ID NO: 4810) |
| | | | | | CAGTGTGTGGTCAACATTTCTCATG | (SEQ ID NO: 4811) |
| | | | | | ATGTTAATTATGCAGTGTTTCCCTC | (SEQ ID NO: 4812) |
| 209796_s_{—} | CNPY2 | MGC156825, | canopy 2 | 10330 | TGGAGCGACCCCATTACGCTAAAGA | (SEQ ID NO: 4813) |
| at | | Tmem4 | homolog | | GAGCGACCCCATTACGCTAAAGATG | (SEQ ID NO: 4814) |
| | | | (zebrafish) | | GACCCCATTACGCTAAAGATGAAAG | (SEQ ID NO: 4815) |
| | | | | | GAGCCAGGATCTCCACTGTGGAGCA | (SEQ ID NO: 4816) |
| | | | | | GAATGGGAAATTGCCCAGGTGGACC | (SEQ ID NO: 4817) |
| | | | | | ACCCCAAGAAGACCATTCAGATGGG | (SEQ ID NO: 4818) |
| | | | | | GACCATTCAGATGGGATCTTTCCGG | (SEQ ID NO: 4819) |
| | | | | | GATGGGATCTTTCCGGATCAATCCA | (SEQ ID NO: 4820) |
| | | | | | GGATCTTTCCGGATCAATCCAGATG | (SEQ ID NO: 4821) |
| | | | | | TTTCCGGATCAATCCAGATGGCAGC | (SEQ ID NO: 4822) |
| | | | | | TCCAGATGGCAGCCAGTCAGTGGTG | (SEQ ID NO: 4823) |
| 209970_x_{—} | CASP1 | ICE, IL1BC, | caspase 1, | 834 | CGAAGGTGATCATCATCCAGGCCTG | (SEQ ID NO: 4824) |
| at | | P45 | apoptosis- | | ACAGCCCTGGTGTGGTGTGGTTTAA | (SEQ ID NO: 4825) |
| | | | related | | GGAAACCTATCTTTACCAACTACAG | (SEQ ID NO: 4826) |
| | | | cysteine | | GAAGGATTTTATCGCTTTCTGCTCT | (SEQ ID NO: 4827) |
| | | | peptidase | | TTTCTGCTCTTCCACACCAGATAAT | (SEQ ID NO: 4828) |
| | | | (interleukin | | GAGACATCCCACAATGGGCTCTGTT | (SEQ ID NO: 4829) |
| | | | 1, beta, | | GAATATGCCTGTTCCTGTGATGTGG | (SEQ ID NO: 4830) |
| | | | convertase) | | TTTCCGCAAGGTTCGATTTTCATTT | (SEQ ID NO: 4831) |
| | | | | | GATGGTAGAGCGCAGATGCCCACCA | (SEQ ID NO: 4832) |
| | | | | | TGACAAGATGTTTCTACCTCTTCCC | (SEQ ID NO: 4833) |
| | | | | | TTCTACCTCTTCCCAGGACATTAAA | (SEQ ID NO: 4834) |
| 210029_at | INDO | CD107B, IDO, | idoleamine- | 3620 | ACCCCCAGCTATCAGACGGTCTGGT | (SEQ ID NO: 4835) |
| | | IDO1 | pyrrole 2,3 | | AGGCCAAAGCAGCGTCTTTCAGTGC | (SEQ ID NO: 4836) |
| | | | diosygenase | | GCTGGTGGAGGACATGCTGCTCAGT | (SEQ ID NO: 4837) |
| | | | | | TGCTCAGTTCCTCCAGGACATGAGA | (SEQ ID NO: 4838) |
| | | | | | GGAACTTCCTGTGCTCATTAGAGTC | (SEQ ID NO: 4839 |
| | | | | | ATTAGAGTCAAATCCCTCAGTCCGT | (SEQ ID NO: 4830) |
| | | | | | ATGACGCCTGTGTGAAAGCTCTGGT | (SEQ ID NO: 4831) |
| | | | | | TGGTCTCCCTGAGGAGCTACCATCT | (SEQ ID NO: 4832) |
| | | | | | GAGCTACCATCTGCAAATCGTGACT | (SEQ ID NO: 4833) |
| | | | | | CATCCTGATTCCTGCAAGCCAGCAG | (SEQ ID NO: 4834) |
| | | | | | GAATAAGACCTCTGAAGACCCTTCA | (SEQ ID NO: 4835) |
| 210976_s_{—} | PFKM | GSD7, | phospho- | 5213 | GCGTAAGAGGGCTCTGGTCTTCCAA | (SEQ ID NO: 4836) |
| at | | MGC8699, | fructokinase, | | TTTGAGCATCGAATCCCCAAGGAAC | (SEQ ID NO: 4837) |
| | | PFK-1, PFK-M, | muscle | | AACTGAGGCCCATCCTCAAAATCCT | (SEQ ID NO: 4838) |
| | | PFKX | | | TGACTTGGACACTTCAGACCATGCC | (SEQ ID NO: 4839) |
| | | | | | TGCCGTCTAAACCTCTCTGGAGTGA | (SEQ ID NO: 4840) |
| | | | | | AGCTCACACCCTAATAAGTCCACAT | (SEQ ID NO: 4841) |
| | | | | | AAGTCCACATCTTCTCAGTGTTTTA | (SEQ ID NO: 4842) |
| | | | | | TATTCTGTACCTTGCAGCCATGACC | (SEQ ID NO: 4843) |
| | | | | | CCCAGCTTTATCTGTCACACAAGGC | (SEQ ID NO: 4844) |
| | | | | | GCTACTGCTAGATATCACTTACTCA | (SEQ ID NO: 4845) |
| | | | | | AAGAGTCTTGGTTCCTCTACTACTT | (SEQ ID NO: 4846) |
| 211122_s_{—} | CXCL11 | H174, I-TAC, | chemokine | 6373 | GAGACTTTTCTATGGTTTTGTGACT | (SEQ ID NO: 4847) |
| at | | IP-9, IP9, | (C-X-C motif) | | GTGACTTTCAACTTTTGTACAGTTA | (SEQ ID NO: 4848) |
| | | MGC102770, | ligand 11 | | GACAATCAGAATTCCACTGCCCAAA | (SEQ ID NO: 4849) |
| | | SCYB11, | | | GGCTGGTTACCATCGGAGTTTACAA | (SEQ ID NO: 4850) |
| | | SCYB9B, b-R1 | | | TGCTTTCACGTTCTTACTTGTTGTA | (SEQ ID NO: 4851) |
| | | | | | TACATTCATGCATTTCTAGGCTAGA | (SEQ ID NO: 4852) |
| | | | | | AGAGAACCTTCTAGATTTGATGCTT | (SEQ ID NO: 4853) |
| | | | | | GTCTCTAGAAGTTATCTGTCTGTAT | (SEQ ID NO: 4854) |
| | | | | | CTGTCTGTATTGATCTTTATGCTAT | (SEQ ID NO: 4855) |
| | | | | | GCTATATTACTATCTGTGGTTACAG | (SEQ ID NO: 4856) |
| | | | | | TACTGGAGTCAAGCCCTTATAAGTC | (SEQ ID NO: 4857) |
| 211340_s_{—} | MCAM | CD146, | melanoma | 4162 | GCCCTCTTGATGGATCACGTAAAAC | (SEQ ID NO: 4858) |
| at | | MUC18 | cell adhesion | | AAGGCAGCGGGGAGCAGACAAAGAT | (SEQ ID NO: 4859) |
| | | | molecule | | AGATGAGGTCTACACTGTCCTTCAT | (SEQ ID NO: 4860) |
| | | | | | GTCTACACTGTCCTTCATGGGGATT | (SEQ ID NO: 4861) |
| | | | | | ACTGTCCTTCATGGGGATTAAAGCT | (SEQ ID NO: 4862) |
| | | | | | GCTATGGTTATATTAGCACCAAACT | (SEQ ID NO: 4863) |
| | | | | | ACCCTAGAAGGGCCCAAATGAGAGA | (SEQ ID NO: 4864) |
| | | | | | ATGAGAGAATGGTACTTAGGGATGG | (SEQ ID NO: 4865) |
| | | | | | GTGTATATATGGTTTTGTCAGGTGT | (SEQ ID NO: 4866) |
| | | | | | GGTTTTGTCAGGTGTGTAAATTTGC | (SEQ ID NO: 4867) |
| | | | | | AATTGTTTCCTTTATATATGTATGT | (SEQ ID NO: 4868) |
| 211370_s_{—} | MAP2K5 | AI324775, | mitogen- | 5607 | CATTCTGATGTCTGGAGCTTAGGAA | (SEQ ID NO: 4869) |
| at | | AI428457, | activated | | CTCCAGCTTCTGCAGTGCATTGTTG | (SEQ ID NO: 4870) |
| | | MEK5, | protein | | TTGTTGATGAGGATTCGCCCGTCCT | (SEQ ID NO: 4871) |
| | | Mapkk5, | kinase kinase | | CCCGTCCTTCCAGTTGGAGAGTTCT | (SEQ ID NO: 4872) |
| | | Prkmk5 | 5 | | GGAGAGTTCTCGGAGCCATTTGTAC | (SEQ ID NO: 4873) |
| | | | | | GGAGCCATTTGTACATTTCATCACT | (SEQ ID NO: 4874) |
| | | | | | ACATTTCATCACTCAGTGTATGCGA | (SEQ ID NO: 4875) |
| | | | | | GCCAAAAGAAAGGCCAGCACCTGAA | (SEQ ID NO: 4876) |
| | | | | | GCACCTGAAGAATTGATGGGCCACC | (SEQ ID NO: 4877) |
| | | | | | AAGAATTGATGGGCCACCCGTTCAT | (SEQ ID NO: 4878) |
| | | | | | ATGATGGAAATGCCGCCGTGGTGTC | (SEQ ID NO: 4879) |
| 211595_s_{—} | MRPS11 | FLJ22512, | mitochondrial | 64963 | ACACAGATCCAGGTAGTCTCTGCTA | (SEQ ID NO: 4880) |
| at | | FLJ23406, | ribosomal | | GTCTCTGCTAGTAATGAGCCCCTTG | (SEQ ID NO: 4881) |
| | | HCC-2 | protein S11 | | AGCACAGACAGCAGGCATAGCCGCA | (SEQ ID NO: 4882) |
| | | | | | AGCCGCAGCGGCGAGAGCTAAACAA | (SEQ ID NO: 4883) |
| | | | | | CAAAAGGGCGTGATCCACATCCGAG | (SEQ ID NO: 4884) |
| | | | | | TTGTCTGCCATGCACGGACTGATCA | (SEQ ID NO: 4885) |
| | | | | | GGACTGATCATGGGCGGCCTGGAAG | (SEQ ID NO: 4886) |
| | | | | | CTCAGCTCCAGTGGGACCTTGTAAA | (SEQ ID NO: 4887) |
| | | | | | GGACCTTGTAAAATGCTCCCTGTCA | (SEQ ID NO: 4888) |
| | | | | | TCCCTGTCAGAGCTCTCCAGAATAT | (SEQ ID NO: 4889) |
| | | | | | GGTGTGCCCCAGAAGTAAGCTTTGC | (SEQ ID NO: 4890) |
| 211951_at | NOLC1 | KIAA0035, | nucleolar and | 9221 | ATTGTGTCTCCTAGTTGGTACCTGG | (SEQ ID NO: 4891) |
| | | NOPP130, | coiled-body | | CCTGGGAGCAATTGACATGCCCCCT | (SEQ ID NO: 4892) |
| | | NOPP140, | phospho- | | AGCTGGCCTGACGGTATGGCTGCAG | (SEQ ID NO: 4893) |
| | | NS5ATP13, | protein | | TGAGCAGTAGCGGTACTCAGCCAGA | (SEQ ID NO: 4894) |
| | | P130 | 1 | | GAGGGAAGAGTCCACAGCTTTCTGG | (SEQ ID NO: 4895) |
| | | | | | GTGGTAGAAAAGTGTGCCCCAAGCC | (SEQ ID NO: 4896) |
| | | | | | CCCCAAGCCTTCATGGACGAGTTAT | (SEQ ID NO: 4897) |
| | | | | | TAGTCTCCTCTTGTTTGGATTCCAT | (SEQ ID NO: 4898) |
| | | | | | GGATTCCATACTTGCTAAATAACCT | (SEQ ID NO: 4899) |
| | | | | | AACCTGGTTTTCCATGTAACTGCCT | (SEQ ID NO: 4900) |
| | | | | | GAAAATGTACTGTTCATGCTGACAC | (SEQ ID NO: 4901) |
| 212463_at | CD59 | 16.3A5, 1F5, | CD59 | 966 | GATGCTGGTGTTGGTGTGACATAAT | (SEQ ID NO: 4902) |
| | | EJ16, EJ30, | molecule, | | ATAATGCTATGGCCAGAACTGAAAC | (SEQ ID NO: 4903) |
| | | EL32, | complement | | GGCTCCCACAGTTAGAAGGCGAAGT | (SEQ ID NO: 4904) |
| | | FLJ38134, | regulatory | | GGCGAAGTCGCACAATAGGCCGTCT | (SEQ ID NO: 4905) |
| | | FLJ92039, | protein | | GCCGTCTGCAAGCTGGGTTAGAGAG | (SEQ ID NO: 4906) |
| | | G344, HRF-20, | | | AGCCAGTAGTGGCTCAGCCTGAGTT | (SEQ ID NO: 4907) |
| | | HRF20, MAC- | | | AACCCACTGGTGCAAGTCCTAGATT | (SEQ ID NO: 4908) |
| | | IP, MACIF, | | | GAAGAACCTGGAGTCTGATGTCCAA | (SEQ ID NO: 4909) |
| | | MEM43, | | | TACCAAAGAGGCTACCGATTCCTTC | (SEQ ID NO: 4910) |
| | | MGC2354, | | | TCTGCACCTTCTAAACCTAGTTCTT | (SEQ ID NO: 4911) |
| | | MIC11, MIN1, | | | GCTTTCCATTACATGAGCTGTCTCA | (SEQ ID NO: 4912) |
| | | MIN2, MIN3, | | | | |
| | | MIRL, MSK21, | | | | |
| | | p18-20 | | | | |
| 212765_at | CAMSAP1L1 | RP11- | calmodulin | 23271 | GAGTTCATTGCTCTCAGTATAAGAT | (SEQ ID NO: 4913) |
| | | 93N17.1, | regulated | | AAGTCTGAGGATTTTCGTCAACCTT | (SEQ ID NO: 4914) |
| | | KIAA1078, | spectrin- | | CGTCAACCTTACTGAAACACACTGG | (SEQ ID NO: 4915) |
| | | MGC150680, | associated | | AACACACTGGTGCTTTCATCATCAG | (SEQ ID NO: 4916) |
| | | MGC150681 | protein 1- | | TTGTCGTGGTTACCAGTGCAGCCTG | (SEQ ID NO: 4917) |
| | | | like 1 | | AAATTCTGCTATTTGACACAGCTTT | (SEQ ID NO: 4918) |
| | | | | | ATGGTTTCATGGACACTGTTGAGCA | (SEQ ID NO: 4919) |
| | | | | | AGTGTATGGTGTGCTTACCTGTCCA | (SEQ ID NO: 4920) |
| | | | | | TACCTGTCCACTCTAGAGCATTGCT | (SEQ ID NO: 4921) |
| | | | | | GAGCATTGCTTACAGGIIIiiiGTT | (SEQ ID NO: 4922) |
| | | | | | TGCTATTTCCTGGTACAGTGTAGTT | (SEQ ID NO: 4923) |
| 213307_at | SHANK2 | CORTBP1, | SH3 and | 22941 | AAAAATTCCAGTCCTCATATCTTTT | (SEQ ID NO: 4924) |
| | | CTTNBP1, | multiple | | CTAGACTTTGCTTCCAATGGTTTCT | (SEQ ID NO: 4925) |
| | | ProSAP1, | ankyrin | | TTCCAGACCACTTTTCCTAGATGAA | (SEQ ID NO: 4926) |
| | | SHANK, | repeat | | GCTGCATGCAGACAGGACTGCCCGT | (SEQ ID NO: 4927) |
| | | SPANK-3 | domains 2 | | GACTGCCCGTGCTTTGTGGGGAATC | (SEQ ID NO: 4928) |
| | | | | | TTCCGCAGATCTCATCACTTTGATT | (SEQ ID NO: 4929) |
| | | | | | GTTTATCGTTCCTGTAACTTGTTCT | (SEQ ID NO: 4930) |
| | | | | | AACTTGTTCTACATTCCACAGTCTT | (SEQ ID NO: 4931) |
| | | | | | ATTCCACAGTCTTTACCGTTTTATG | (SEQ ID NO: 4932) |
| | | | | | CAACAATCCCTGTCCATTGATTCCA | (SEQ ID NO: 4933) |
| | | | | | GAACTCTTTGTTCATGCCAATTTTG | (SEQ ID NO: 4934) |
| 213656_s_{—} | KLC1 | KLC, KNS2, | kinesin light | 3831 | CAGCAGCAGTGGCCTGGAAGACGCC | (SEQ ID NO: 4935) |
| at | | KNS2A, | chain 1 | | GCAGTGGCCTGGAAGACGCCACCGC | (SEQ ID NO: 4936) |
| | | MGC15245 | | | TTCTCCTGCGTCTGTGTGCATAGGA | (SEQ ID NO: 4937) |
| | | | | | CCTGCGTCTGTGTGCATAGGACATG | (SEQ ID NO: 4938) |
| | | | | | GTCTGTGTGCATAGGACATGATACT | (SEQ ID NO: 4939) |
| | | | | | GACATGATACTAATAACCACACGGC | (SEQ ID NO: 4940) |
| | | | | | AATAACCACACGGCTGGCGTGACCT | (SEQ ID NO: 4941) |
| | | | | | CCATGTGTAACTTCCTCACGTTGTG | (SEQ ID NO: 4942) |
| | | | | | TGTAACTTCCTCACGTTGTGTGCGA | (SEQ ID NO: 4943) |
| | | | | | ACTTCCTCACGTTGTGTGCGATAAC | (SEQ ID NO: 4944) |
| | | | | | GTGTGCGATAACGTATTTTATTGTA | (SEQ ID NO: 4945) |
| 213895_at | EMP1 | CL-20, EMP-1, | epithelial | 2012 | AAGGACTGGTATCTTTCTGTGAGCA | (SEQ ID NO: 4946) |
| | | TMP | membrane | | GATAAAGACTGCATATCCTTGTGTC | (SEQ ID NO: 4947) |
| | | | protein 1 | | ATTGCATTGATTCTTGATGCTTTCT | (SEQ ID NO: 4948) |
| | | | | | GATGCTTTCTTAGAGGCCTACATGA | (SEQ ID NO: 4949) |
| | | | | | GATTTCTTAGATTGCTCTGATAAAC | (SEQ ID NO: 4950) |
| | | | | | AGAGGGAACTTTTGTCAGACTCTGC | (SEQ ID NO: 4951) |
| | | | | | CAGACTCTGCAACAAACTCCTAGCT | (SEQ ID NO: 4952) |
| | | | | | AACTCCTAGCTCTATCCAGAGTGTC | (SEQ ID NO: 4953) |
| | | | | | TCCAGAGTGTCCTCTGCTGCTAAGA | (SEQ ID NO: 4954) |
| | | | | | GTATCTTTCTCCTCAAAAGCCTGGA | (SEQ ID NO: 4955) |
| | | | | | CACCCCAGTCCAGCCATGAGGGTAT | (SEQ ID NO: 4956) |
| 213925_at | C1orf95 | RP11-9C4.1, | Chromosome | 375057 | ATGAGGGTATTTCCAGCCTATTCCA | (SEQ ID NO: 4957) |
| | | DKFZp761P211 | 1 open | | ATTTCCAGCCTATTCCAATGACTGA | (SEQ ID NO: 4958) |
| | | | reading | | CAATGACTGAAACCCTTCTTGGAAG | (SEQ ID NO: 4959) |
| | | | frame 95 | | TAATGTCGGGGACGTAGGGACGCTT | (SEQ ID NO: 4960) |
| | | | | | CTAGGGGCCAAGCAGGGTTCCAGAT | (SEQ ID NO: 4961) |
| | | | | | AGCCTTCTGCCAGCTTTAAAACCCT | (SEQ ID NO: 4962) |
| | | | | | AAAACCCTTCACTCAAGCAGCATTA | (SEQ ID NO: 4964) |
| | | | | | AGCAGCATTACTGTACATGCAATGA | (SEQ ID NO: 4965) |
| | | | | | CATACACACACCTTCCTATTATAAA | (SEQ ID NO: 4966) |
| | | | | | AAGCATGACATCTCTTTGGCATTCC | (SEQ ID NO: 4967) |
| 214106_s_{—} | GMDS | RP1- | GDP- | 2762 | CAGAAGCTGAACTGGAAGCCCCGGG | (SEQ ID NO: 4968) |
| at | | 118B18.1, | mannose | | GGTCGCTTTCGATGAGCTGGTGAGG | (SEQ ID NO: 4969) |
| | | GMD | 4,6- | | GTGGAGCTCATGAGGACAAACCCCA | (SEQ ID NO: 4970) |
| | | | dehydratase | | TGCAGACGCGCTGCGGGGATGGGGA | (SEQ ID NO: 4971) |
| | | | | | CCGCCGAGGTTTGTAGCAGCCGGGA | (SEQ ID NO: 4972) |
| | | | | | TTGTAGCAGCCGGGATGTGACCCTC | (SEQ ID NO: 4973) |
| | | | | | TTTGGGTCGCTTTGCGTTTGTCGAA | (SEQ ID NO: 4974) |
| | | | | | TTTGTCGAAGCCTCCTCTGAATGGC | (SEQ ID NO: 4975) |
| | | | | | GTCGAAGCCTCCTCTGAATGGCTTT | (SEQ ID NO: 4976) |
| | | | | | AATCACATTCACTTTACTTGAAATT | (SEQ ID NO: 4977) |
| | | | | | CAAATTGTGGGGCCTTCAAATTGTT | (SEQ ID NO: 4978) |
| 214167_s_{—} | RPLP0/// | L10E, | ribosomal | 220717 | CCATTGCCCCATGTGAAGTCACTGT | (SEQ ID NO: 4979) |
| at | RPLP0-like | MGC111226, | protein | 6175 | CCCCATGTGAAGTCACTGTGCCAGC | (SEQ ID NO: 4980) |
| | | MGC88175, | large, P0/// | | CCAGCCCAGAACACTGGTCTCGGGC | (SEQ ID NO: 4981) |
| | | P0, PRLP0, | ribosomal | | AGCCCAGAACACTGGTCTCGGGCCC | (SEQ ID NO: 4982) |
| | | RPP0/// | protein P0- | | GAACACTGGTCTCGGGCCCGAGAAG | (SEQ ID NO: 4983) |
| | | | like | | TGGTCTCGGGCCCGAGAAGACCTCC | (SEQ ID NO: 4984) |
| | | | | | TTTTTCCAGGCTTTAGGTATCACCA | (SEQ ID NO: 4985) |
| | | | | | GGTATCACCACTAAAATCTCCAGGG | (SEQ ID NO: 4986) |
| | | | | | CACCACTAAAATCTCCAGGGGCACC | (SEQ ID NO: 4987) |
| | | | | | TAAAATCTCCAGGGGCACCATTGAA | (SEQ ID NO: 4988) |
| | | | | | ATCTCCAGGGGCACCATTGAAATCC | (SEQ ID NO: 4989) |
| 214175_x_{—} | PDLIM4 | RIL | PDZ and LIM | 8572 | CTACTACTATACGGCTGCGAGAAGA | (SEQ ID NO: 4990) |
| at | | | domain 4 | | ATACGGCTGCGAGAAGACGACAGAA | (SEQ ID NO: 4991) |
| | | | | | GACGACAGAAGGGCTACGACGTGGT | (SEQ ID NO: 4992) |
| | | | | | AGAAGGGCTACGACGTGGTGGCGGT | (SEQ ID NO: 4993) |
| | | | | | GGTGTACCCCAATGCCAAGGTGGAA | (SEQ ID NO: 4994) |
| | | | | | GTACCCCAATGCCAAGGTGGAACTC | (SEQ ID NO: 4995) |
| | | | | | ATGCCAAGGTGGAACTCGTCTGAGC | (SEQ ID NO: 4996) |
| | | | | | AGGTGGAACTCGTCTGAGCTGGGAC | (SEQ ID NO: 4997) |
| | | | | | CCCTGCTCGGCCGGTGTAAATATGT | (SEQ ID NO: 4998) |
| | | | | | TGCTCGGCCGGTGTAAATATGTTTC | (SEQ ID NO: 4999) |
| | | | | | GTGTAAATATGTTTCACCCTGTCCC | (SEQ ID NO: 5000) |
| 214620_x_{—} | PAM | PAL, PHM | peptidyl- | 5066 | CTCCTCCTGAAAACCAAGCTTTGAT | (SEQ ID NO: 5001) |
| at | | | glycine | | CAGAATGTCAGATTCCTTTCCCTTT | (SEQ ID NO: 5002) |
| | | | alpha- | | GACTGTACACACTTTATTTACTTCG | (SEQ ID NO: 5003) |
| | | | amidating | | AGTTCATAACAGTGCCATTGTCTTT | (SEQ ID NO: 5004) |
| | | | monooxygenase | | TAGACTAGAGAAACCGTCCTCTTTT | (SEQ ID NO: 5005) |
| | | | | | TCCTCTTTTTCCATCATAATTCTAA | (SEQ ID NO: 5006) |
| | | | | | GATTTGCCCATTTACACTTTTGAGA | (SEQ ID NO: 5007) |
| | | | | | GTGTTTTCAGAGCATTAGCTGTCAG | (SEQ ID NO: 5008) |
| | | | | | TAGCTGTCAGTGTATTTTCCAGTTT | (SEQ ID NO: 5009) |
| | | | | | TTCCAGTTTTGGGTATTGCAGATTT | (SEQ ID NO: 5010) |
| | | | | | TGCAGATTTTACATACAACTTTTAT | (SEQ ID NO: 5011) |
| 215034_s_{—} | TM4SF1 | H-L6, L6, | transmembrane | 4071 | TATGACTGCTAAAAGAACCAACCCA | (SEQ ID NO: 5012) |
| at | | M3S1, TAAL6 | 4 L six | | AAAGAACCAACCCAGGACAGAGCCA | (SEQ ID NO: 5013) |
| | | | family | | ACCCAGGACAGAGCCACAATCTTCC | (SEQ ID NO: 5014) |
| | | | member 1 | | CAGGACAGAGCCACAATCTTCCTCT | (SEQ ID NO: 5015) |
| | | | | | ACAGAGCCACAATCTTCCTCTATTT | (SEQ ID NO: 5016) |
| | | | | | TTTCACTTGTATTCATTTGTAAAAC | (SEQ ID NO: 5017) |
| | | | | | TTTGTATTAGTGTAACATACTCCCC | (SEQ ID NO: 5018) |
| | | | | | AAACGCCTGTAAAGACTGGCATCTT | (SEQ ID NO: 5019) |
| | | | | | GCCTGTAAAGACTGGCATCTTCACA | (SEQ ID NO: 5020) |
| | | | | | GACTGGCATCTTCACAGGATGTCAG | (SEQ ID NO: 5021) |
| | | | | | ATCTTCACAGGATGTCAGTGTTTAA | (SEQ ID NO: 5022) |
| 215440_s_{—} | BEX4 | RP4- | BEX family | 56271 | GACAACCATTATGACTTTTGCCTCA | (SEQ ID NO: 5023) |
| at | | 635G19.2, | member 4 | | TTGCCTCATACCTTGAATCCTAAAA | (SEQ ID NO: 5024) |
| | | BEXL1, | | | AAAGTTTTCGCTGAGGTTAATGTGA | (SEQ ID NO: 5025) |
| | | FLJ10097 | | | GTGAACACTGCTTTACAAGCTTGTA | (SEQ ID NO: 5026) |
| | | | | | GTGATTTACTTTTTCTGTAAGCCTT | (SEQ ID NO: 5027) |
| | | | | | GTTTACACTTACCAGTTTCTAATGG | (SEQ ID NO: 5028) |
| | | | | | GTCTCAGCCTAAAAGTTACGGTCAG | (SEQ ID NO: 5029) |
| | | | | | TACGGTCAGCATGGCAATTCACCTA | (SEQ ID NO: 5030) |
| | | | | | CTGGACTCAAAATCTCAGTTGTCTT | (SEQ ID NO: 5031) |
| | | | | | TGTAGGACCTATCGTCCAGACTCAC | (SEQ ID NO: 5032) |
| | | | | | CGTCCAGACTCACAGAGTGGGGCTC | (SEQ ID NO: 5033) |
| 216220_s_{—} | ADORA1 | RDC7 | adenosine | 134 | GACCAGGTGTCTAGAGGCAACAGTG | (SEQ ID NO: 5034) |
| at | | | A1 receptor | | TAGAGGCAACAGTGTTCTGAGCCCC | (SEQ ID NO: 5035) |
| | | | | | GGCTGGGAGAAGGTGCTTGGGCTTC | (SEQ ID NO: 5036) |
| | | | | | TGCTTGGGCTTCTGCGGTGAGGCAG | (SEQ ID NO: 5037) |
| | | | | | GGGCTTCTGCGGTGAGGCAGGGGAG | (SEQ ID NO: 5038) |
| | | | | | GAGGCAGGGGAGTCTGCTTGTCTTA | (SEQ ID NO: 5039) |
| | | | | | GGGGAGTCTGCTTGTCTTAGATGTT | (SEQ ID NO: 5040) |
| | | | | | GTCTTAGATGTTGGTGGTGCAGCCC | (SEQ ID NO: 5041) |
| | | | | | CAGCCCCAGGACCAAGCTTAAGGAG | (SEQ ID NO: 5042) |
| | | | | | GAGGAGAGCATCTGCTCTGAGACGG | (SEQ ID NO: 5043) |
| | | | | | GGAAGGAGAGAGGTTGAGGATGCAC | (SEQ ID NO: 5044) |
| 216682_s_{—} | FAM48A | RGD1307812 | Family with | 55578 | CAGAAAAACTCATTGGTTACCATCA | (SEQ ID NO: 5045) |
| at | | | sequence | | GTTACCATCAGAGTTTGCTAGGGCA | (SEQ ID NO: 5046) |
| | | | similarity 48, | | GGGCATCAGATTCTTACTCTGAAGA | (SEQ ID NO: 5047) |
| | | | member A | | AGAATATAATATTTATCCTGCCCTT | (SEQ ID NO: 5048) |
| | | | | | TCCTGCCCTTCTTGTTATGAACTGT | (SEQ ID NO: 5049) |
| | | | | | GGAAAATTTCCAGCTAATAGGTGCA | (SEQ ID NO: 5050) |
| | | | | | GTAGTGAATCTAGACAGCAGTATTT | (SEQ ID NO: 5051) |
| | | | | | TCTGATCAAACCCACTGATTGAAGA | (SEQ ID NO: 5052) |
| | | | | | GACAGTCAATTATTGTGTACCTCCT | (SEQ ID NO: 5053) |
| | | | | | GTGTACCTCCTGGTTTGATGCAAGA | (SEQ ID NO: 5054) |
| | | | | | GATGCAAGAGACAGTACACAACAGT | (SEQ ID NO: 5055) |
| 217731_s_{—} | ITM2B | RP11- | integral | 9445 | TTCCACCTTTATGTTTTAATATCCT | (SEQ ID NO: 5056) |
| at | | 196P14.1, | membrane | | TGTTTTAATATCCTAGGCATCTGCT | (SEQ ID NO: 5057) |
| | | ABRI, BRI, | protein 2B | | CCTAGGCATCTGCTGTAATAATATT | (SEQ ID NO: 5058) |
| | | BRI2, | | | ATAACTTGTGTTACTAATTTGTATA | (SEQ ID NO: 5059) |
| | | BRICD2B, | | | TAATTTGTATAACCCATATCTGTGC | (SEQ ID NO: 5060) |
| | | E25B, E3-16, | | | TGTATAACCCATATCTGTGCAATGG | (SEQ ID NO: 5061) |
| | | FBD | | | AAGTTGTTTAACTAGACTGCGTGTT | (SEQ ID NO: 5062) |
| | | | | | ACTAGACTGCGTGTTGTTTTTCCCG | (SEQ ID NO: 5063) |
| | | | | | GAATAGTTTGGTTCTTCAAATCTTA | (SEQ ID NO: 5064) |
| | | | | | CAAATCTTAAGAGAATCCACATAAA | (SEQ ID NO: 5065) |
| | | | | | AAAATTCACTTCTATATATACAATG | (SEQ ID NO: 5066) |
| 217861_s_{—} | PREB | MGC3467, | prolactin | 10113 | GTCCACAGCTGAGGTTGCCTCTGAC | (SEQ ID NO: 5067) |
| at | | SEC12 | regulatory | | AAAAGGCTTGGCTATGGCCCTGTGT | (SEQ ID NO: 5068) |
| | | | element | | CTTCGTCATCTGTGGATCCATCCAG | (SEQ ID NO: 5069) |
| | | | binding | | GAACAGCGGTATCTGAAGCCCAGGC | (SEQ ID NO: 5070) |
| | | | | | AGAGGCTCCAGAGTTGAGCTTGTCC | (SEQ ID NO: 5071) |
| | | | | | GATGCCCAAGAGCCTGGAGGCACTG | (SEQ ID NO: 5072) |
| | | | | | CTGCAGAAACAGTTTCTCCTCCTCC | (SEQ ID NO: 5073) |
| | | | | | ACATGAAGCCCCTGGCATTTGCTGG | (SEQ ID NO: 5074) |
| | | | | | GATGGGTAGCACTAAGCCAGCTGGC | (SEQ ID NO: 5075) |
| | | | | | AGCCAGCTGGCCTAAAGATGCAATA | (SEQ ID NO: 5076) |
| | | | | | ATGAACCTCAGCCCATTAGGCAGGA | (SEQ ID NO: 5077) |
| 217870_s_{—} | CMPK1 | RP11-511i2.1, | cytidine | 51727 | GGAATGAGTTCTTATCTAGTGTTGC | (SEQ ID NO: 5078) |
| at | | CMK, CMPK, | monophosphate | | TCTTATCTAGTGTTGCAGGCCAGCA | (SEQ ID NO: 5079) |
| | | UMK, UMP- | (UMP-CMP) | | GGTTTCGAGAGCATTCCTACTCACA | (SEQ ID NO: 5080) |
| | | CMPK, UMPK | kinase 1 | | GAGCATTCCTACTCACATAAGTGAA | (SEQ ID NO: 5081) |
| | | | cytosolic | | TGAGACTAGGTGCTTTGCTTCCTTT | (SEQ ID NO: 5082) |
| | | | | | GCTTCCTTTCATCAGGTATCTTTCT | (SEQ ID NO: 5083) |
| | | | | | AGGTATCTTTCTGTGGCATTTGAGA | (SEQ ID NO: 5084) |
| | | | | | TTACTAAATTATGAGGCTTTGCTTT | (SEQ ID NO: 5085) |
| | | | | | TAACTAGTTTTGTCATTCCATTTGT | (SEQ ID NO: 5086) |
| | | | | | GATACAGTCACCAAGAATGTTTTGA | (SEQ ID NO: 5087) |
| | | | | | AGACCCCAATTTAAGCCTTGCTTAT | (SEQ ID NO: 5088) |
| 217933_s_{—} | LAP3 | LAP, LAPEP, | leucine | 51056 | ATGTACAGCTGCAGCATTCCTGAAA | (SEQ ID NO: 5089) |
| at | | PEPS | aminopeptidase | | GTAACTCATCCTAAGTGGGCACATT | (SEQ ID NO: 5090) |
| | | | 3 | | TGGGCACATTTAGACATAGCAGGCG | (SEQ ID NO: 5091) |
| | | | | | GCAGGCGTGATGACCAACAAAGATG | (SEQ ID NO: 5092) |
| | | | | | GAAGTTCCCTATCTACGGAAAGGCA | (SEQ ID NO: 5093) |
| | | | | | GAAAGGCATGACTGGGAGGCCCACA | (SEQ ID NO: 5094) |
| | | | | | GGCCCACAAGGACTCTCATTGAGTT | (SEQ ID NO: 5095) |
| | | | | | GAGTTCTTACTTCGTTTCAGTCAAG | (SEQ ID NO: 5096) |
| | | | | | GACAATGCTTAGTTCAGATACTCAA | (SEQ ID NO: 5097) |
| | | | | | AGAACTTCCTAATCACTTTTCAGAG | (SEQ ID NO: 5098) |
| | | | | | GAAAATTACTATGCACTTGTCAGAA | (SEQ ID NO: 5099) |
| 218126_at | FAM82C | RMD3; | family with | 55177 | CTTTTTTCCTTAGACCTTGCTGAGA | (SEQ ID NO: 5100) |
| | | FAM82A2; | sequence | | ACCACACAAATCTGTCTCCTGGGTC | (SEQ ID NO: 5101) |
| | | FAM82C; | similarity 82, | | TACCACTCCCCATTAGTTAATTTAT | (SEQ ID NO: 5102) |
| | | hRMD-3; | member C | | GTTGTTCTCTCCCTTGAGTAATCTC | (SEQ ID NO: 5103) |
| | | ptpip51; | | | ACCTGCCCCAGGATTACACATGGGT | (SEQ ID NO: 5104) |
| | | FLJ10579 | | | GGTAGAGCCTGCAAGACCTGAGACC | (SEQ ID NO: 5105) |
| | | | | | GACCTGAGACCTTCCAATTGCTGGT | (SEQ ID NO: 5106) |
| | | | | | CAGAGTGGCCCACAGACATTGCTTT | (SEQ ID NO: 5107) |
| | | | | | GAATTCTACCTGTATTCCAGGGCTG | (SEQ ID NO: 5108) |
| | | | | | AGGGCTGGACCACTTGATAACTTCC | (SEQ ID NO: 5109) |
| | | | | | AACTTCCAGTGTCCTGGCAGCTTTT | (SEQ ID NO: 5110) |
| 218434_s_{—} | AACS | ACSF1, | acetoacetyl- | 65985 | GGATCTCTTTATTGCACAGACTGAA | (SEQ ID NO: 5111) |
| at | | FLJ12389, | CoA | | TGTGAAGCAGTGGGTGTCCACCCGT | (SEQ ID NO: 5112) |
| | | FLJ41251, | synthetase | | TGTGGTCACCGAGTGAGGACCCTCC | (SEQ ID NO: 5113) |
| | | SUR-5 | | | CTGCCTCCACCTGAGAGTTGCTAGG | (SEQ ID NO: 5114) |
| | | | | | GTTGCTAGGGGGTTCTTGTCGAGAT | (SEQ ID NO: 5115) |
| | | | | | TGTCGAGATCATGTCATCAGCACCC | (SEQ ID NO: 5116) |
| | | | | | CCTAAGTCAAGTCACGGGTTTCCAT | (SEQ ID NO: 5117) |
| | | | | | GGGTTTCCATAGCCAGGCAGTTGGT | (SEQ ID NO: 5118) |
| | | | | | TGGTATGTACAATTCAGTTCAGCGT | (SEQ ID NO: 5119) |
| | | | | | TCAGCGTATGAACTTGTATCTCTAA | (SEQ ID NO: 5120) |
| | | | | | GTATCTCTAATCTGATGTCCATTTT | (SEQ ID NO: 5121) |
| 218551_at | RP5- | IIP45; | invasion | 60672 | CCACCGGCGAAAGAGCTTTGACGCC | (SEQ ID NO: 5122) |
| | 1077B9.4 | FLJ12438; | inhibitory | | CGAAAGAGCTTTGACGCCTCTGACA | (SEQ ID NO: 5123) |
| | | FLJ38609 | protein 45 | | GTCTGAGAAAAGCTCAGCCCCCAGG | (SEQ ID NO: 5124) |
| | | | | | TAGGTTGGGCAGGTGGGTGGACCCA | (SEQ ID NO: 5125) |
| | | | | | AGGTGGGTGGACCCAAGCTTGTCTG | (SEQ ID NO: 5126) |
| | | | | | TTGTCTGCTGCCTGAGTTCCAGAGA | (SEQ ID NO: 5127) |
| | | | | | GTCTGCTGCCTGAGTTCCAGAGAGG | (SEQ ID NO: 5128) |
| | | | | | GAGTTCCAGAGAGGGAGGACCCTGG | (SEQ ID NO: 5129) |
| | | | | | CAGAGAGGGAGGACCCTGGGGTGGA | (SEQ ID NO: 5130) |
| | | | | | GGGGTGGAGGGTGAGGGATTCTGTG | (SEQ ID NO: 5131) |
| | | | | | GTGGAGGGTGAGGGATTCTGTGGAA | (SEQ ID NO: 5132) |
| 218622_at | NUP37 | FLJ22618, | mucleoporin | 79023 | CAATCCGGTTTTATGATCTTTTGGC | (SEQ ID NO: 5133) |
| | | MGC5585, p37 | 37 kDa | | GGCCCAACAGGCTATTTTATCTCTT | (SEQ ID NO: 5134) |
| | | | | | ACACCTTCAAAGTTGGAGCCGTTGC | (SEQ ID NO: 5135) |
| | | | | | TATTACTCGGTCCAGTTATCCTCAA | (SEQ ID NO: 5136) |
| | | | | | GAGACCTGTTCACATGGATCGAGCC | (SEQ ID NO: 5137) |
| | | | | | GAGCCTGCTTATTCAGGTGGTCCAC | (SEQ ID NO: 5138) |
| | | | | | TGCAACCACTGGTTATCCTGGCAAA | (SEQ ID NO: 5139) |
| | | | | | TTCATCATTTAGGACACCCTCAGCC | (SEQ ID NO: 5140) |
| | | | | | ACTCTCCCTCTGTGTGTAATTGGAG | (SEQ ID NO: 5141) |
| | | | | | GAGACCACAAGCTGTTGTTTTGGGT | (SEQ ID NO: 5142) |
| | | | | | AAGTGTTTTCTGTACCTTAGATTCA | (SEQ ID NO: 5143) |
| 218686_s_{—} | RHBDF1 | C16orf8, | rhomboid 5 | 64285 | TGGATTGACAACTTTGCCCACATCT | (SEQ ID NO: 5144) |
| at | | Dist1, | homolog 1 | | CACATCTCGGGGTTCATCAGTGGCC | (SEQ ID NO: 5145) |
| | | EGFR-RS, | (Drosophila) | | AAACGCTGCCAGATCATCATCTTTC | (SEQ ID NO: 5146) |
| | | FLJ2235, | | | ATCTTTCAGGTGGTCTTCCTGGGCC | (SEQ ID NO: 5147) |
| | | FLJ22357, | | | ATCCCCTTCACTGACAAGTTCTGTG | (SEQ ID NO: 5148) |
| | | gene-89, gene- | | | GAAGTACGAACTGGACGCTCAGCTC | (SEQ ID NO: 5149) |
| | | 90, hDist1 | | | TACAGGAGTCACCTGCTCCATGTGG | (SEQ ID NO: 5150) |
| | | | | | GGCCTGTTTCCTGAACACAGACCTC | (SEQ ID NO: 5151) |
| | | | | | ACTGCCGGGCATTTATTATACTACT | (SEQ ID NO: 5152) |
| | | | | | TACTTCCTGTCATAACCTTCTAACT | (SEQ ID NO: 5153) |
| | | | | | CTTGACGACCACCTCATGTGGCCAA | (SEQ ID NO: 5154) |
| 219148_at | PBK | SPK; TOPK; | PDZ binding | 55872 | AGCATACTATGCAGCGTTGGGAACT | (SEQ ID NO: 5155) |
| | | Nori-3; | kinase | | CAGCGTTGGGAACTAGGCCACCTAT | (SEQ ID NO: 5156) |
| | | FLJ14385 | | | TGAACTCTTCTCTGTATGCACTAAT | (SEQ ID NO: 5157) |
| | | | | | AGACCCTAAAGATCGTCCTTCTGCT | (SEQ ID NO: 5158) |
| | | | | | ATGTCTAGTGATCATCTCAGCTGAA | (SEQ ID NO: 5159) |
| | | | | | GTGTGGCTTGCGTAAATAACTGTTT | (SEQ ID NO: 5160) |
| | | | | | GAGGACCATAGTTTCTTGTTAACAT | (SEQ ID NO: 5161) |
| | | | | | AAGCACTTGGAATTGTACTGGGTTT | (SEQ ID NO: 5162) |
| | | | | | GTACTTTGATACTGCTCATGCTGAC | (SEQ ID NO: 5163) |
| | | | | | TGCTCATGCTGACTTAAAACACTAG | (SEQ ID NO: 5164) |
| | | | | | GGATCTACTGACATTAGCACTTTGT | (SEQ ID NO: 5165) |
| 219258_at | TIPIN | FLJ20516 | TIMELESS | 54962 | GAACATGATGTCACTTCTACTGAAT | (SEQ ID NO: 5166) |
| | | | interacting | | CTACTGAATTAGATCCCTTTCTGAC | (SEQ ID NO: 5167) |
| | | | protein | | GATCCCTTTCTGACAAACTTATCTG | (SEQ ID NO: 5168) |
| | | | | | GTTAATGAATACACCCAGGGCACAC | (SEQ ID NO: 5169) |
| | | | | | CCCAGGGCACACACGGTTGAAGAGG | (SEQ ID NO: 5170) |
| | | | | | AACGAAGACATTCTGGACAATCCAT | (SEQ ID NO: 5171) |
| | | | | | GAAACACTGCTGGACCAGTCTTTTA | (SEQ ID NO: 5172) |
| | | | | | GCAACAGCAACTTGATGCTACATCC | (SEQ ID NO: 5173) |
| | | | | | TGTATCTGTTAAGTCATCGTCCTGC | (SEQ ID NO: 5174) |
| | | | | | TTAAGTCATCGTCCTGCAAGCTTGG | (SEQ ID NO: 5175) |
| | | | | | GCAAGCTTGGCGTTACTATGTATTT | (SEQ ID NO: 5176) |
| 219315_s_{—} | TMEM204 | C16orf30, | transmembrane | 79652 | CTACGTGGAGTCACCATGCTGAGTC | (SEQ ID NO: 5177) |
| at | | CLP24, | protein | | CACACCTGCTATCGTGGAACAGCCT | (SEQ ID NO: 5178) |
| | | FLJ20898, | 204 | | ACGGGATGAGTCTGGGTGACCTCTG | (SEQ ID NO: 5179) |
| | | MGc111564 | | | GTGCGTTTACTGTTATGTCGGTCAT | (SEQ ID NO: 5180) |
| | | | | | GTCGGTCATATGTCTGTACGTGTCG | (SEQ ID NO: 5181) |
| | | | | | TCCAGCTTTCCTGGTTAGCGCAACG | (SEQ ID NO: 5182) |
| | | | | | ACAGGTTAGGTGGCGCGAGGCTGCC | (SEQ ID NO: 5183) |
| | | | | | CGCTCCGCTTTGCTTTGGGATTAAT | (SEQ ID NO: 5184) |
| | | | | | TCTGCATCTGCTGAGAGGGGCACCC | (SEQ ID NO: 5185) |
| | | | | | CCCCAGCCATATCTTACACTTTGGT | (SEQ ID NO: 5186) |
| | | | | | GAATCGTGTTCGTGTCTGTTTTGCT | (SEQ ID NO: 5187) |
| 219649_at | ALG6 | 0 | asparagine- | 29929 | TTTAGTATGCTACCTCTTCTATTGA | (SEQ ID NO: 5188) |
| | | | linked | | AAGGATGAACTCCTAATGCCCTCTG | (SEQ ID NO: 5189) |
| | | | glycosylation | | CTAATGCCCTCTGTTGTGACAACAA | (SEQ ID NO: 5190) |
| | | | 6 homolog | | TATAGCTTGTGTAACTTCCTTTTCA | (SEQ ID NO: 5191) |
| | | | (S. | | TTGTTTCTTATCTCAGTCATCACTA | (SEQ ID NO: 5192) |
| | | | cerevisiae, | | CACTATGGTGCTTCTGACGTTGATG | (SEQ ID NO: 5193) |
| | | | alpha-1,3- | | GACGTTGATGACTGTCACACTGGAT | (SEQ ID NO: 5194) |
| | | | glucosyl- | | TGGATCCTCCTCAGAAACTACCGGA | (SEQ ID NO: 5195) |
| | | | transferase) | | ATTGGTGTGTTTTGTATCTTGCTTG | (SEQ ID NO: 5196) |
| | | | | | ATCTTGCTTGAACTTCCTGTTCTTC | (SEQ ID NO: 5197) |
| | | | | | ACTTCCTGTTCTTCTTGGTATACTT | (SEQ ID NO: 5198) |
| 219789_at | NPR3 | ANPRC, | natriuretic | 4883 | TTATGATTAATCACCATCTGCCTCC | (SEQ ID NO: 5199) |
| | | GUCY2B, | peptide | | CCAGGCCTTTCATCTCATGACAAAC | (SEQ ID NO: 5200) |
| | | NPRC | receptor | | ATCGTGTCACTCTGTTAAATGTTCA | (SEQ ID NO: 5201) |
| | | | C/guanylate | | TGTTCATACTGTTTCAAGCCCATAT | (SEQ ID NO: 5202) |
| | | | cyclase C | | TGTTGTCTCCATATCTTGATGGCTT | (SEQ ID NO: 5203) |
| | | | (atrionatri- | | GGCTTTTGGGAGCATTTCACACAAG | (SEQ ID NO: 5204) |
| | | | uretic peptide | | CAGGTTTGTGGTTGAGGACTTCTCT | (SEQ ID NO: 5205) |
| | | | receptor C) | | GGACTTCTCTGTCCGATGTCTACAT | (SEQ ID NO: 5206) |
| | | | | | GTCTACATTCAGGTTCTGACTTCAT | (SEQ ID NO: 5207) |
| | | | | | TCCCTGTCTTTTTCAGTGTCTCATA | (SEQ ID NO: 5208) |
| | | | | | GTCTCATAAACGCTACTCTGGATTG | (SEQ ID NO: 5209) |
| 220232_at | SCD5 | ACOD4, | stearoyl-CoA | 79966 | ACCTGTACACGTTGATTATTTTTGT | (SEQ ID NO: 5210) |
| | | FADS4, | desaturase 5 | | GAGTTTAAGCTATCATTTCCAGATG | (SEQ ID NO: 5211) |
| | | FLJ21032, | | | GGAGAGTTCCATAGATGTCTGTTAG | (SEQ ID NO: 5212) |
| | | HSCD5, | | | ATCCTTGTTAATTTTCTGTCTTGTT | (SEQ ID NO: 5213) |
| | | SCD2, SCD4 | | | TAAAGTCTCCCACTATTATTATATG | (SEQ ID NO: 5214) |
| | | | | | GTCTAAGTCTCCTTGTAGGTCTCTA | (SEQ ID NO: 5215) |
| | | | | | GGGTGCTCCTGTATTAGGTGCATAC | (SEQ ID NO: 5216) |
| | | | | | TTATATAATGCCCTTCTTTGTCTTT | (SEQ ID NO: 5217) |
| | | | | | TAAATATTCCTGTATCCCTTTATTT | (SEQ ID NO: 5218) |
| | | | | | GTCTTTGCACGTGAGGTGGGTCTCC | (SEQ ID NO: 5219) |
| | | | | | CTGAATACAGCACACCGATGGGTTT | (SEQ ID NO: 5220) |
| 220441_at | FLJ13236 | FLJ13236, wus | hypothetical | 79962 | GTCAGCTGGCTTACCAGGTTTTGGG | (SEQ ID NO: 5221) |
| | | | protein | | TTACCAGGTTTTGGGCCTCTCAGAA | (SEQ ID NO: 5222) |
| | | | FLJ13236 | | TACATCGGAGTTACCAGGAGCTAGT | (SEQ ID NO: 5223) |
| | | | | | GGAGCTAGTGAAGGTCTGGCACCCA | (SEQ ID NO: 5224) |
| | | | | | AGATCCAGGCTGCGTATGAAGTCCT | (SEQ ID NO: 5225) |
| | | | | | GTCCTGAGTCAACCCAGGAAGCCCT | (SEQ ID NO: 5226) |
| | | | | | CCTGAGGACTGACTCTTCCTAGCAG | (SEQ ID NO: 5227) |
| | | | | | CTAGCAGAGCTGGGCAACTTGTCCC | (SEQ ID NO: 5228) |
| | | | | | GGCAACTTGTCCCAAATCTAGCTTT | (SEQ ID NO: 5229) |
| | | | | | CCAAATCTAGCTTTGCCCACGAATG | (SEQ ID NO: 5230) |
| | | | | | TTGCCCACGAATGGCATCCCAACAG | (SEQ ID NO: 5231) |
| 220596_at | GPATCH4 | RP11- | G patch | 54865 | TCTTCTGCAAATTTCCCTTTAAACT | (SEQ ID NO: 5232) |
| | | 284F21.4, | domain | | TCCTCCTTTAATTAAATGGACTACA | (SEQ ID NO: 5233) |
| | | GPATC4 | containing 4 | | GGACTACATGGGGGGGTAATCCAGG | (SEQ ID NO: 5234) |
| | | | | | GCTTTCCCCTGGGCAGATGGTAGGC | (SEQ ID NO: 5235) |
| | | | | | GGCAGATGGTAGGCAGGGAGCTGGG | (SEQ ID NO: 5236) |
| | | | | | ATGGGGATCACCTTGAGTCCATCCA | (SEQ ID NO: 5237) |
| | | | | | GAGGTGATATCATCCTTTTAAGAAG | (SEQ ID NO: 5238) |
| | | | | | GTAGAAAGTGCCCAAACAGCCATCA | (SEQ ID NO: 5239) |
| | | | | | CAAACAGCCATCAGTTCCATCCTGA | (SEQ ID NO: 5240) |
| | | | | | TCAGTTCCATCCTGACTGCTTGTTA | (SEQ ID NO: 5241) |
| | | | | | TGACTGCTTGTTAAGAATTCTCTGC | (SEQ ID NO: 5242) |
| 220794_at | GREM2 | PRDC; | gremlin 2, | 64388 | CGCCCCCTATTTGTGGACTAAAGAT | (SEQ ID NO: 5243) |
| | | DAND3; | cysteine knot | | GATGAACTCTGGTGTGCATGCTATT | (SEQ ID NO: 5244) |
| | | CKTSF1B2 | superfamily, | | AAGGTCTCTATATTAACGCTGGTTT | (SEQ ID NO: 5245) |
| | | | homolog | | TCACATGTTTTGACTCTGGCTTGCA | (SEQ ID NO: 5246) |
| | | | (Xenopus | | TGGCTTGCAGCACCATTCGGAGTAA | (SEQ ID NO: 5247) |
| | | | laevis) | | GGAGTAAGGATGACAGCAGGCCCAG | (SEQ ID NO: 5248) |
| | | | | | AGCAGGCCCAGAAGGTGTTTTACTC | (SEQ ID NO: 5249) |
| | | | | | CAGCAATTCACTGTCATGCACCTAT | (SEQ ID NO: 5250) |
| | | | | | TTCACTGACGTTACTAAGCATTGCA | (SEQ ID NO: 5251) |
| | | | | | AAACCGTATACTACTTTCACTGCCA | (SEQ ID NO: 5252) |
| | | | | | CTACTTTCACTGCCATGACACTAGG | (SEQ ID NO: 5253) |
| 220960_x_{—} | RPL22 | EAP, HBP15, | ribosomal | 6146 | AATTCTGATGTCGTACCTAAGGCTT | (SEQ ID NO: 5254) |
| at | | HBP15/L22 | protein L22 | | ACCTAAGGCTTGTCCATCTTTGTTG | (SEQ ID NO: 5255) |
| | | | | | TTGTTGGAGGTGCCATGGCTCCTGT | (SEQ ID NO: 5256) |
| | | | | | TGAAGTTCACTCTTGATTGCACCCA | (SEQ ID NO: 5257) |
| | | | | | ATTGCACCCACCCTGTAGAAGATGG | (SEQ ID NO: 5258) |
| | | | | | GGAGCAAGAGCAAGATCACCGTGAC | (SEQ ID NO: 5259) |
| | | | | | ATCACCGTGACATCCGAGGTGCCTT | (SEQ ID NO: 5260) |
| | | | | | GAATAATCTACGTGACTGGTTGCGC | (SEQ ID NO: 5261) |
| | | | | | CTGGTTGCGCGTAGTTGCTAACAGC | (SEQ ID NO: 5262) |
| | | | | | TACGAATTACGTTACTTCCAGATTA | (SEQ ID NO: 5263) |
| | | | | | TTTATCCTTGTATCTCTGCAGTGTG | (SEQ ID NO: 5264) |
| 221221_s_{—} | KLHL3 | FLJ40871, | kelch-like 3 | 26249 | GGATAATTACAGCCTCATAGCCCCT | (SEQ ID NO: 5265) |
| at | | KIAA1129, | (Drosophila) | | AGCCTTCGTCATGGGACTCAGTGAC | (SEQ ID NO: 5266) |
| | | MGC44594 | | | GCACAGGACTGTGGCATTTGCAGCA | (SEQ ID NO: 5267) |
| | | | | | AAATCACCCTAGTGCCATGTTTGGT | (SEQ ID NO: 5268) |
| | | | | | TAGAGGACCCTTGTTGTACTTCTTG | (SEQ ID NO: 5269) |
| | | | | | GTCCCTGTGGTTGTCATAGCCAGTC | (SEQ ID NO: 5270) |
| | | | | | GTCAGACTTGATCACTGACACCCCG | (SEQ ID NO: 5271) |
| | | | | | GACACCCCGTACAACATATTGCATA | (SEQ ID NO: 5272) |
| | | | | | TAAGATCCTCGATCTGGTGTTCTCT | (SEQ ID NO: 5273) |
| | | | | | GTTCTCTGCGTGGCTGTTAGGGACT | (SEQ ID NO: 5274) |
| | | | | | GAACACTTGTCACATGCTTGATCAG | (SEQ ID NO: 5275) |
| 221265_s_{—} | C15orf44 | DKFZp564O1664 | chromosome | 81556 | TCACCTCTAGAATAGCCACCCAAAG | (SEQ ID NO: 5276) |
| at | | | 15 open | | AGACCTTCCTGAGGCTGCCTCAGAA | (SEQ ID NO: 5277) |
| | | | reading | | CCTCAGAAGCACCACTTGCTGTTTT | (SEQ ID NO: 5278) |
| | | | frame 44 | | GAAGATAGCTGCTGTTCCCATGATG | (SEQ ID NO: 5279) |
| | | | | | TCCCATGATGGGCACATTTCCTGAG | (SEQ ID NO: 5280) |
| | | | | | ACTGATGAGCATAGAGCACCCCTGC | (SEQ ID NO: 5281) |
| | | | | | CAGGAAACCTGACCGGCAGGGGCTC | (SEQ ID NO: 5282) |
| | | | | | CTCTGGCTTCCTGAAAGCTTCACCT | (SEQ ID NO: 5283) |
| | | | | | TCTTCCCTCGTTTATATCTCAACTG | (SEQ ID NO: 5284) |
| | | | | | GCAACTCAGACTGTCTGGAGCTTGC | (SEQ ID NO: 5285) |
| | | | | | GTTTCAGAACTTTCCTTAGGACTTG | (SEQ ID NO: 5286) |
| 221654_s_{—} | USP3 | MGC129878, | ubiquitin | 9960 | TCTCACTTTGAGGCACATTTACATC | (SEQ ID NO: 5287) |
| at | | MGC129879, | specific | | TACATCAATGCTTTTGTTCCTCTCA | (SEQ ID NO: 5288) |
| | | SIH003, UBP | peptidase 3 | | AAGCAAGATGTGTTCCTTATTGTGA | (SEQ ID NO: 5289) |
| | | | | | GTGAAGAGCGACACAACTGCCTGCT | (SEQ ID NO: 5290) |
| | | | | | GCTGCCTTTCCACAGCTATAATGGA | (SEQ ID NO: 5291) |
| | | | | | TGGACATCAGGTTGACTCTAAATCA | (SEQ ID NO: 5292) |
| | | | | | GTGTGCACAATACTTGTGGCCCACA | (SEQ ID NO: 5293) |
| | | | | | GACCAGGTAATTACTGCTTGTCTCT | (SEQ ID NO: 5294) |
| | | | | | TTACTGCTTGTCTCTCAAGGCTGCT | (SEQ ID NO: 5295) |
| | | | | | TCAAGGCTGCTGTCTTTATCAGCAC | (SEQ ID NO: 5296) |
| | | | | | GGTTCAGTTGTACTTGTCCTGCAAA | (SEQ ID NO: 5297) |
| 221747_at | TNS1 | MSTP091, | tensin 1 | 7145 | CCTCTGTCCTCAAATGTCCAAAATG | (SEQ ID NO: 5298) |
| | | DKFZp586K0617, | | | TTGGAGGACCTCTGTTCATATCCCA | (SEQ ID NO: 5299) |
| | | MGC88584, | | | GGCTCTTGCCAGCAGTGGAGTTACT | (SEQ ID NO: 5300) |
| | | MST091, | | | GAGTTACTGTAGAGGGATGTCCCAA | (SEQ ID NO: 5301) |
| | | MST122, | | | AGAGGGATGTCCCAAGCTTGTTTTC | (SEQ ID NO: 5302) |
| | | MST127, | | | TTTGAAACTCTCCTGTGTCTGTGTT | (SEQ ID NO: 5303) |
| | | MSTP122, | | | GTGTGTGAGAGCACATCAGTGTGTG | (SEQ ID NO: 5304) |
| | | MSTP127, | | | GCACATCAGTGTGTGCAGGCTGTGT | (SEQ ID NO: 5305) |
| | | MSRA6, TNS | | | TCCCTTCAGACCCATCATTGAGAAC | (SEQ ID NO: 5306) |
| | | | | | CAGGCCCTCTGCGGGGGAAACAAGA | (SEQ ID NO: 5307) |
| | | | | | GGGAAACAAGATCACCCAGCATCCT | (SEQ ID NO: 5308) |
| 222357_at | ZBTB20 | RP23- | zinc finger | 26137 | CGTCGACCTCCCTATGGATTGTTAA | (SEQ ID NO: 5309) |
| | | 238M18.1, | and BTB | | GGATTGTTAAGGGCCCTGTGCATAT | (SEQ ID NO: 5310) |
| | | 1300017A20Rik, | domain | | GGCCCTGTGCATATTTGCATGTAGC | (SEQ ID NO: 5311) |
| | | 7330412A13Rik, | containing 20 | | TGCATGTAGCCAAAGATTCAGTCTC | (SEQ ID NO: 5312) |
| | | A930017C21Rik, | | | GATTCAGTCTCTTGCAGGAACTCTG | (SEQ ID NO: 5313) |
| | | D16Wsu73e, | | | GCAGGAACTCTGGTTGTCAACTGGT | (SEQ ID NO: 5314) |
| | | DPZF, HOF, | | | TCAACTGGTGATCTTGCCTTTGTCT | (SEQ ID NO: 5315) |
| | | ODA-8S, | | | AGGACAATTTTACCATTCTCTCCTG | (SEQ ID NO: 5316) |
| | | Oda8, Zfp288 | | | TTTTAGTGCCGGTACATGAATTTGT | (SEQ ID NO: 5317) |
| | | | | | TATTCTTTATAATTCCCTTTGAAGT | (SEQ ID NO: 5318) |
| | | | | | GGGGAATCTAAAACCGACCAGATGT | (SEQ ID NO: 5319) |
| 34408_at | RTN2 | NSP2, NSPL1 | reticulon 2 | 6253 | TCCGAGCTAAAATCCCAGGGACCGG | (SEQ ID NO: 5320) |
| | | | | | TTACCTGAGCGACCAGGACTACATT | (SEQ ID NO: 5321) |
| | | | | | GCCTGCTGGGACTTGTAGTTGCCTA | (SEQ ID NO: 5322) |
| | | | | | TGCTGGGACTTGTAGTTGCCTAGAC | (SEQ ID NO: 5323) |
| | | | | | TGGGACTTGTAGTTGCCTAGACAGG | (SEQ ID NO: 5324) |
| | | | | | TGTAGTTGCCTAGACAGGGCACCAC | (SEQ ID NO: 5325) |
| | | | | | GTAGTTGCCTAGACAGGGCACCACC | (SEQ ID NO: 5326) |
| | | | | | AGGCGTTGGTGTCTCCTGGATGCTA | (SEQ ID NO: 5327) |
| | | | | | GGCGTTGGTGTCTCCTGGATGCTAC | (SEQ ID NO: 5328) |
| | | | | | GCGTTGGTGTCTCCTGGATGCTACT | (SEQ ID NO: 5329) |
| | | | | | CGTTGGTGTCTCCTGGATGCTACTA | (SEQ ID NO: 5330) |
| | | | | | GGGAGGCCTGAGCTTGGATTTACAC | (SEQ ID NO: 5331) |
| | | | | | GGAGGCCTGAGCTTGGATTTACACT | (SEQ ID NO: 5332) |
| | | | | | GGCCTGAGCTTGGATTTACACTGTA | (SEQ ID NO: 5333) |
| | | | | | CTGAGCTrGGATTTACACTGTAATA | (SEQ ID NO: 5334) |
| | | | | | CTTGGATTTACACTGTAATAAAGAC | (SEQ ID NO: 5335) |
| 58780_s_{—} | FLJ10357 | SOLO | hypothetical | 55701 | CTGGAGTCCCTGGTGACTCCATTCT | (SEQ ID NO: 5336) |
| at | | | protein | | GAGTCCCTGGTGACTCCATTCTGAG | (SEQ ID NO: 5337) |
| | | | FLJ10357 | | GTCCCTGGTGACTCCATTCTGAGGT | (SEQ ID NO: 5338) |
| | | | | | TCCCTGGTGACTCCATTCTGAGGTG | (SEQ ID NO: 5339) |
| | | | | | CTGGTGACTCCATTCTGAGGTGTCA | (SEQ ID NO: 5340) |
| | | | | | GGTGACTCCATTCTGAGGTGTCACA | (SEQ ID NO: 5341) |
| | | | | | GACTCCATTCTGAGGTGTCACAAGC | (SEQ ID NO: 5342) |
| | | | | | CTCCATTCTGAGGTGTCACAAGCAA | (SEQ ID NO: 5343) |
| | | | | | TCCATTCTGAGGTGTCACAAGCAAT | (SEQ ID NO: 5344) |
| | | | | | ATTCTGAGGTGTCACAAGCAATGAA | (SEQ ID NO: 5345) |
| | | | | | TTCTGAGGTGTCACAAGCAATGAAG | (SEQ ID NO: 5346) |
| | | | | | TCTGAGGTGTCACAAGCAATGAAGC | (SEQ ID NO: 5347) |
| | | | | | TGAGGTGTCACAAGCAATGAAGCTA | (SEQ ID NO: 5348) |
| | | | | | AGGTGTCACAAGCAATGAAGCTATG | (SEQ ID NO: 5349) |
| | | | | | GTGTGACAGGGGAACCGTAGACTTT | (SEQ ID NO: 5350) |
| | | | | | TGTGACAGGGGAACCGTAGACTTTA | (SEQ ID NO: 5351) |
| 64408_s_{—} | CALML4 | MGC4809, | calmodulin- | 91860 | GAAAGAAATTCTTCTAGCCATGTTG | (SEQ ID NO: 5352) |
| at | | NY-BR-20 | like 4 | | ATTCTTCTAGCCATGTTGATGGTGG | (SEQ ID NO: 5353) |
| | | | | | CTTCTAGCCATGTTGATGGTGGACA | (SEQ ID NO: 5354) |
| | | | | | CTAGCCATGTTGATGGTGGACAAGG | (SEQ ID NO: 5355) |
| | | | | | CGGTCAAAACTCACGAGTCTGGGGG | (SEQ ID NO: 5356) |
| | | | | | GGTCAAAACTCACGAGTCTGGGGGA | (SEQ ID NO: 5357) |
| | | | | | GATCACCCTTCCTGGACGGGACTAT | (SEQ ID NO: 5358) |
| | | | | | CACCCTTCCTGGACGGGACTATTGA | (SEQ ID NO: 5359) |
| | | | | | TTCCTGGACGGGACTATTGAAGGAG | (SEQ ID NO: 5360) |
| | | | | | CTCCCCTGGGCCTGAAAACTTGGAG | (SEQ ID NO: 5361) |
| | | | | | CCTGGGCCTGAAAACTTGGAGCAAT | (SEQ ID NO: 5362) |
| | | | | | CTGGGCCTGAAAACTTGGAGCAATT | (SEQ ID NO: 5363) |
| | | | | | AACACAGTGGCAAGACAACATTACC | (SEQ ID NO: 5364) |
| | | | | | AACATTACCCAACTATAGAAGAGAG | (SEQ ID NO: 5365) |
| | | | | | TACCCAACTATAGAAGAGAGGCTAA | (SEQ ID NO: 5366) |
| | | | | | ACCCAACTATAGAAGAGAGGCTAAC | (SEQ ID NO: 5367) |
| 290132_s_{—} | COMMD4 | FLJ20452 | COMM | | TCTGCAGCCTACGCATGAATAGGTT | (SEQ ID NO: 5368) |
| at | | | domain | | GCAGCCTACGCATGAATAGGTTGGC | (SEQ ID NO: 5369) |
| | | | containing 4 | | GCATGAATAGGTTGGCAGGTGTGGG | (SEQ ID NO: 5370) |
| | | | | | AGCCTGCTGCAATCCGTGGAAGAGC | (SEQ ID NO: 5371) |
| | | | | | GCTGCAATCCGTGGAAGAGCCCATG | (SEQ ID NO: 5372) |
| | | | | | GCAATCCGTGGAAGAGCCCATGGTG | (SEQ ID NO: 5373) |
| | | | | | TCCGTGGAAGAGCCCATGGTGCACC | (SEQ ID NO: 5374) |
| | | | | | CCATGTCCCTCTCAGCAGACAAGTT | (SEQ ID NO: 5375) |
| | | | | | ATGTCCCTCTCAGCAGACAAGTTCC | (SEQ ID NO: 5376) |
| | | | | | TCCCTCTCAGCAGACAAGTTCCAGG | (SEQ ID NO: 5377) |
| | | | | | CCTCTCAGCAGACAAGTTCCAGGTC | (SEQ ID NO: 5378) |

All gene probe set identification numbers and Entrez Gene identification numbers of Table 2 are hereby incorporated by reference in their entirety

In accordance with this aspect of the present invention, a good disease prognosis expression profile consists of genes, from the collection of 101 genes informative of colon cancer disease outcome, having expression levels that are below that of an average tumor sample expression level that are selected from the group consisting of ACTN1, ADORA1, ARHGAP8, LOC553158, BEX4, C1orf95, C3orf63, CAMSAP1L1, CD59, CNPY2, DBN1, FAM48A, FLJ10357, GPATCH4, GRB10, GREM2, HDAC5, HOXA4, ITM2B, KLC1, KLF12, KLHL3, NPR3, PAM, PBX2, PDLIM4, PIR, RGL2, RHBDF1, RP5-1077B9.4, RTN2, SCD5, SHANK2, SVIL, TAPBP, TIPIN, TM4SF1, TMEM204, TNS1, TUSC3 and ZBTB20. A good disease expression profile further consists of genes having expression levels that are above the average tumor sample expression level that are selected from the group consisting of NARS, WDR1, WARS, CCT4, ATP5B, SORD, UBE2L6, PSME2, AIP, RRM2, LRRC41, CCT2, TAF9, CCNB2, RFC5, IDE, MAD2L1, PSMA4, NDUFC1, IVD, PPIH, NEO1, CXCL10, FXN, GABBR1, COMMD4, DFFB, GLMN, CASP7, ATP5G3, DDOST, CYB561, NR2F1, WDR68, CXCL2, CASP1, INDO, PFKM, CXCL11, MCAM, MAP2K5, MRPS11, NOLC1, EMP1, GMDS, RPLP0, RPLP0-like, PREB, CMPK1, LAP3, FAM82C, AACS, NUP37, PBK, ALG6, FLJ13236, RPL22, C15orf44, USP3 and CALML4.

Also in accordance with this aspect of the present invention, a bad disease prognosis expression profile consists of genes from the collection of 101 genes informative of colon cancer disease outcome, having expression levels below that of an average tumor sample expression level that are selected from the group consisting of NARS, WDR1, WARS, CCT4, ATP5B, SORD, UBE2L6, PSME2, AIP, RRM2, LRRC41, CCT2, TAF9, CCNB2, RFC5, IDE, MAD2L1, PSMA4, NDUFC1, IVD, PPIH, NEO1, CXCL10, FXN, GABBR1, COMMD4, DFFB, GLMN, CASP7, ATP5G3, DDOST, CYB561, NR2F1, WDR68, CXCL2, CASP1, INDO, PFKM, CXCL11, MCAM, MAP2K5, MRPS11, NOLC1, EMP1, GMDS, RPLP0, RPLP0-like, PREB, CMPK1, LAP3, FAM82C, AACS, NUP37, PBK, ALG6, FLJ13236, RPL22, C15orf44, USP3 and CALML4. A bad disease expression profile further consists of genes having expression levels that are above the average tumor sample expression level that are selected from the group consisting of ACTN1, ADORA1, ARHGAP8, LOC553158, BEX4, C1orf9S, C3orf63, CAMSAP1L1, CD59, CNPY2, DBN1, FAM48A, FLJ10357, GPATCH4, GRB10, GREM2, HDAC5, HOXA4, ITM2B, KLC1, KLF12, KLHL3, NPR3, PAM, PBX2, PDLIM4, PIR, RGL2, RHBDF1, RP5-1077B9.4, RTN2, SCD5, SHANK2, SVIL, TAPBP, TIPIN, TM4SF1, TMEM204, TNS1, TUSC3 and ZBTB20.

Determining the prognosis of a subject having colon cancer using the gene expression data of the present invention, involves calculating the percentage of genes analyzed having expression levels associated with a good disease prognosis expression profile and the percentage of genes analyzed having expression levels associated with a bad disease prognosis expression profile in the sample from the subject. A favorable prognosis for the subject exists when greater than 20%, more preferably, greater than 25%, and most preferably, greater than 30% of the genes analyzed have expression levels associated with a good disease prognosis expression profile and less than 30%, more preferably, less than 25%, and most preferably, less than 20% of the genes analyzed have expression levels associated with a bad disease prognosis expression profile. An unfavorable prognosis for the subject exists when greater than 20%, more preferably, greater than 25%, and most preferably, greater than 30% of the genes analyzed have expression levels associated with a bad disease prognosis expression profile and less than 30%, more preferably, less than 25%, and most preferably, less than 20% of the genes analyzed have expression levels associated with a good disease prognosis expression profile.

A biological sample obtained from the subject having colon cancer in accordance with the methods of the present invention can be any biological tissue, fluid, or cell sample. Typical biological samples include, but are not limited to, sputum, blood, blood cells (e.g., white cells), tissue or fine needle biopsy samples, urine, stool, peritoneal fluid, and pleural fluid, or cells therefrom. Biological samples may also include sections of tissues such as frozen sections taken for histological purposes. In a preferred embodiment of the present invention, the biological sample obtained from the subject having colon cancer is a population of primary colon cancer cells. The colon cancer cells can be derived from a stage I, II, III, or IV colon cancer tumor.

Methods of isolating RNA and protein from biological samples for use in the methods of the present invention are readily known in the art. Protein preparation can be carried out using any method that produces analyzable protein. For example, the sample cells or tissue can be lysed in a protein lysis buffer (e.g. 50 mM Tris-HCl (pH, 6.8), 100 mM DTT, 100 μg/ml PMSF, 2% SDS, 10% glycerol, 1 μg/ml each of pepstatin A, leupeptin, and aprotinin, and 1 mM sodium orthovanadate) and sheared with a 22-gauge needle. Other methods of protein isolation that are suitable for use in carrying out the methods of the present invention are fully described in DENNISON C., A GUIDE TO PROTEIN ISOLATION (Kluwer Academic Publishers 2003), which is hereby incorporated by reference in its entirety. The protein content of the samples can be estimated using the Lowry, Bradford, or bicinchoninic acid assays or any commercially available assay based on the aforementioned techniques.

Methods of isolation and purification of nucleic acids suitable for use in carrying out the methods of the present invention are described in detail in LABORATORY TECHNIQUES IN BIOCHEMISTRY AND MOLECULAR BIOLOGY: HYBRIDIZATION WITH NUCLEIC ACID PROBES, PART I. THEORY AND NUCLEIC ACID PREPARATION (P. Tijssen ed., Elsevier 1993) which is incorporated herein by reference. Total RNA can be isolated from a given sample using, for example, an acid guanidinium-phenol-chloroform extraction, a guanidinium isothiocyanate-ultracentrifugation method, or a lithium chloride-SDS-urea method. PolyA⁺ mRNA can be isolated using oligo(dT) column chromatography or (dT)n magnetic beads (See e.g., SAMBROOK AND RUSSELL, MOLECULAR CLONING: A LABORATORY MANUAL (Cold Springs Laboratory Press, 1989) or CURRENT PROTOCOLS IN MOLECULAR BIOLOGY (Fred M. Ausubel et al. eds., 1992) which are hereby incorporated by reference in their entirety). See also WO/2000024939 to Dong et al. which is hereby incorporated by reference in its entirety, for complexity management and other nucleic acid sample preparation techniques.

It may be desirable to amplify the nucleic acid sample prior to detecting gene expression. One of skill in the art will appreciate that a method which maintains or controls for the relative frequencies of the amplified nucleic acids to achieve quantitative amplification should be used.

Typically, methods for amplifying nucleic acids employ a polymerase chain reaction (PCR) (See e.g., PCR TECHNOLOGY: PRINCIPLES AND APPLICATIONS FOR DNA AMPLIFICATION (Henry Erlich ed., Freeman Press 1992); PCR PROTOCOLS: A GUIDE TO METHODS AND APPLICATIONS (Michael Innis ed., Academic Press 1990); Mattila et al., “Fidelity of DNA Synthesis by the Thermococcus litoralis DNA Polymerase—An Extremely Heat Stable Enzyme with Proofreading Activity,” Nucleic Acids Res. 19:4967-73 (1991); Eckert et al., “DNA polymerase fidelity and the polymerase chain reaction,” PCR Methods and Applications 1:17-24 (1991); and U.S. Pat. Nos. 4,683,202, 4,683,195, 4,800,159 4,965,188, and 5,333,675, all to Mullis et al., which are hereby incorporated by reference in their entireties for all purposes. The sample can also be amplified on an array as described in U.S. Pat. No. 6,300,070 to Boles, which is incorporated herein by reference.

Other suitable amplification methods include the ligase chain reaction (LCR) (e.g., Wu et al., “The Ligation Amplification Reaction (LAR)—Amplification of Specific DNA Sequences Using Sequential Rounds of Template-Dependent Ligation,” Genomics 4:560-9 (1989), Landegren et al., “A Ligase-Mediated Gene Detection Technique,” Science 24-1:1077-80 (1988) and Barringer et al., “Blunt-End and Single-Strand Ligations by Escherichia coli Ligase: Influence on an In Vitro Amplification Scheme,” Gene 89:117-22 (1990), which are hereby incorporated by reference in their entirety); transcription amplification (Kwoh et al., “Transcription-Based Amplification System and Detection of Amplified Human Immunodeficiency Virus Type 1 with a Bead-Based Sandwich Hybridization Format,” Proc. Natl. Acad. Sci. USA 86:1173-7 (1989) and WO88/10315 to Gingeras, which are hereby incorporated by reference in their entirety); self-sustained sequence replication (Guatelli et al., “Isothermal, In Vitro Amplification of Nucleic Acids by a Multienzyme Reaction Modeled After Retroviral Replication,” Proc. Nat. Acad. Sci. USA 87:1874-8 (1990) and WO90/06995 to Gingeras, which are hereby incorporated by reference in their entirety); selective amplification of target polynucleotide sequences (U.S. Pat. No. 6,410,276 to Burg et al, which is hereby incorporated by reference in its entirety); consensus sequence primed polymerase chain reaction (CP-PCR) (U.S. Pat. No. 5,437,975 to McClelland, which is hereby incorporated by reference in its entirety); arbitrarily primed polymerase chain reaction (AP-PCR) (U.S. Pat. Nos. 5,413,909 to Bassam, and 5,861,245 to McClelland which are hereby incorporated by reference in their entirety); and nucleic acid based sequence amplification (NABSA) (See U.S. Pat. Nos. 5,409,818, 5,554,517, and 6,063,603 all to Davey, which are hereby incorporated by reference in their entirety). Other amplification methods that may be used are described in U.S. Pat. Nos. 5,242,794 to Whiteley; 5,494,810 to Barany; and 4,988,617 to Landegren, which are hereby incorporated by reference in their entirety.

As described herein, detecting the “expression level” of a gene can be achieved by measuring any suitable value that is representative of the gene expression level. The measurement of gene expression levels can be direct or indirect. A direct measurement involves measuring the level or quantity of RNA or protein. An indirect measurement may involve measuring the level or quantity of cDNA, amplified RNA, DNA, or protein; the activity level of RNA or protein; or the level or activity of other molecules (e.g., a metabolite) that are indicative of the foregoing. The measurement of expression can be a measurement of the absolute quantity of a gene product. The measurement can also be a value representative of the absolute quantity, a normalized value (e.g., a quantity of gene product normalized against the quantity of a reference gene product), an averaged value (e.g., average quantity obtained at different time points or from different tumor cell samples from a subject, or average quantity obtained using different probes, etc.), or a combination thereof.

When it is desirable to measure the expression level of a gene by measuring the level of protein expression, any protein hybridization or immunodetection based assay known in the art can be used. In a protein hybridization based assay, an antibody or other agent that selectively binds to a protein is used to detect the amount of that protein expressed in a sample. For example, the level of expression of a protein can be measured using methods that include, but are not limited to, western blot, immunoprecipitation, enzyme-linked immunosorbent assay (ELISA), radioimmunoassay (RIA), fluorescent activated cell sorting (FACS), immunohistochemistry, immunocytochemistry, or any combination thereof. Also, antibodies, aptamers, or other ligands that specifically bind to a protein can be affixed to so-called “protein chips” (protein microarrays) and used to measure the level of expression of a protein in a sample. Alternatively, assessing the level of protein expression can involve analyzing one or more proteins by two-dimensional gel electrophoresis, mass spectroscopy (MS), matrix-assisted laser desorption/ionization-time of flight-MS (MALDI-TOF), surface-enhanced laser desorption ionization-time of flight (SELDI-TOF), high performance liquid chromatography (HPLC), fast protein liquid chromatography (FPLC), multidimensional liquid chromatography (LC) followed by tandem mass spectrometry (MS/MS), protein chip expression analysis, gene chip expression analysis, and laser densitometry, or any combinations of these techniques.

Measuring gene expression by quantifying mRNA expression can be achieved using any commonly used method known in the art including northern blotting and in situ hybridization (Parker et al., “mRNA: Detection by in Situ and Northern Hybridization,” Methods in Molecular Biology 106:247-283 (1999), which is hereby incorporated by reference in its entirety); RNAse protection assay (Hod et al., “A Simplified Ribonuclease Protection Assay,” Biotechniques 13:852-854 (1992), which is hereby incorporated by reference in its entirety); reverse transcription polymerase chain reaction (RT-PCR) (Weis et al., “Detection of Rare mRNAs via Quantitative RT-PCR,” Trends in Genetics 8:263-264 (1992), which is hereby incorporated by reference in its entirety); and serial analysis of gene expression (SAGE) (Velculescu et al., “Serial Analysis of Gene Expression,” Science 270:484-487 (1995); and Velculescu et al., “Characterization of the Yeast Transcriptome,” Cell 88:243-51 (1997), which is hereby incorporated by reference in its entirety). Alternatively, antibodies may be employed that recognize specific duplexes, including DNA duplexes, RNA duplexes, and DNA-RNA hybrid duplexes or DNA-protein duplexes.

In a preferred embodiment of the present invention, mRNA expression is measured using a nucleic acid amplification assay that is a semi-quantitative or quantitative real-time polymerase chain reaction (RT-PCR) assay. Because RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MLV-RT), although others are also known and suitable for this purpose. The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. An exemplary PCR amplification system using Taq polymerase is TaqMan® PCR (Applied Biosystems, Foster City, Calif.). Taqman® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect the nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, the ABI PRISM 7700® Sequence Detection System®(Perkin-Elmer-Applied Biosystems, Foster City, Calif., USA), or the Lightcycler (Roche Molecular Biochemicals, Mannheim, Germany).

In addition to the TaqMan primer/probe system, other quantitative methods and reagents for real-time PCR detection that are known in the art (e.g. SYBR green, Molecular Beacons, Scorpion Probes, etc.) are suitable for use in the methods of the present invention.

To minimize errors and the effect of sample-to-sample variation, RT-PCR is usually performed using an internal standard. The ideal internal standard is expressed at a constant level among different tissues, and is unaffected by colon cancer. RNAs most frequently used to normalize patterns of gene expression are mRNAs for the housekeeping genes glyceraldehyde-3-phosphate-dehydrogenase (GAPDH) and β-actin.

Real time PCR is compatible both with quantitative competitive PCR, where internal competitor for each target sequence is used for normalization and quantitative comparative PCR using a normalization gene contained within the sample, or a housekeeping gene for RT-PCR. For further details see, e.g., Heid et al., “Real Time Quantitative PCR,” Genome Research 6:986-994 (1996), which is incorporated by reference in its entirety.

In a preferred embodiment of the present invention, the expression levels of genes informative of colon cancer prognosis are detected using an array-based technique. These arrays, also commonly referred to as “microarrays” or “chips” have been generally described in the art, see e.g., U.S. Pat. Nos. 5,143,854 to Pirrung et al.; 5,445,934 to Fodor et al.; 5,744,305 to Fodor et al.; 5,677,195 to Winkler et al.; 6,040,193 to Winkler et al.; 5,424,186 to Fodor et al., which are all hereby incorporated by reference in their entirety. A microarray comprises an assembly of distinct polynucleotide or oligonucleotide probes immobilized at defined positions on a substrate. Arrays are formed on substrates fabricated with materials such as paper, glass, plastic (e.g., polypropylene, nylon), polyacrylamide, nitrocellulose, silicon, optical fiber or any other suitable solid or semi-solid support, and configured in a planar (e.g., glass plates, silicon chips) or three-dimensional (e.g., pins, fibers, beads, particles, microtiter wells, capillaries) configuration. Probes forming the arrays may be attached to the substrate by any number of ways including (i) in situ synthesis (e.g., high-density oligonucleotide arrays) using photolithographic techniques (see Fodor et al., “Light-Directed, Spatially Addressable Parallel Chemical Synthesis,” Science 251:767-773 (1991); Pease et al., “Light-Generated Oligonucleotide Arrays for Rapid DNA Sequence Analysis,” Proc. Natl. Acad. Sci. U.S.A. 91:5022-5026 (1994); Lockhart et al., “Expression Monitoring by Hybridization to High-Density Oligonucleotide Arrays,” Nature Biotechnology 14:1675 (1996); and U.S. Pat. Nos. 5,578,832 to Trulson; 5,556,752 to Lockhart; and 5,510,270 to Fodor, which are hereby incorporated by reference in their entirety); (ii) spotting/printing at medium to low-density (e.g., cDNA probes) on glass, nylon or nitrocellulose (Schena et al., “Quantitative Monitoring of Gene Expression Patterns with a Complementary DNA Microarray,” Science 270:467-470 (1995), DeRisi et al, “Use of a cDNA Microarray to Analyse Gene Expression Patterns in Human Cancer,” Nature Genetics 14:457-460 (1996); Shalon et al., “A DNA Microarray System for Analyzing Complex DNA Samples Using Two-Color Fluorescent Probe Hybridization,” Genome Res. 6:639-645 (1996); and Schena et al., “Proc. Natl. Acad. Sci. U.S.A. 93:10539-11286) (1995), which are hereby incorporated by reference in their entirety); (iii) masking (Maskos et al., “Oligonucleotide Hybridizations on Glass Supports: A Novel Linker for Oligonucleotide Synthesis and Hybridization Properties of Oligonucleotides Synthesised In Situ,” Nuc. Acids. Res. 20:1679-1684 (1992), which is hereby incorporated by reference in its entirety); and (iv) dot-blotting on a nylon or nitrocellulose hybridization membrane (see e.g., SAMBROOK AND RUSSELL, MOLECULAR CLONING: A LABORATORY MANUAL (Cold Springs Laboratory Press, 1989), which is hereby incorporated by reference in its entirety). Probes may also be noncovalently immobilized on the substrate by hybridization to anchors, by means of magnetic beads, or in a fluid phase such as in microtiter wells or capillaries. The probe molecules are generally nucleic acids such as DNA, RNA, PNA, and cDNA but may also include proteins, polypeptides, oligosaccharides, cells, tissues and any permutations thereof which can specifically bind the target molecules.

Fluorescently labeled cDNA for hybridization to the array may be generated through incorporation of fluorescent nucleotides by reverse transcription of RNA extracted from colon cancer tumor tissue of interest. Labeled cDNA applied to the array hybridizes with specificity to each nucleic acid probe spotted on the array. After stringent washing to remove non-specifically bound cDNA, the array is scanned by confocal laser microscopy or by another detection method, such as a CCD camera. Quantitation of hybridization of each arrayed element allows for assessment of corresponding mRNA abundance. With dual color fluorescence, separately labeled cDNA samples generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., “Parallel Human Genome Analysis: Microarray-Based Expression Monitoring of 1000 Genes,” “Proc. Natl. Acad. Sci. USA 93(20):10614-9 (1996), which is hereby incorporated by reference in its entirety).

When the use of microarray technology is desired, the expression levels of genes informative of colon cancer prognosis can be detected using commercially available arrays comprising nucleic acid probes, where at least five of the nucleic acid probes are complementary at least a portion of a nucleotide sequence (i.e., an RNA transcript or DNA nucleotide sequence) of a gene in the group of 176, 71, or 101 genes informative of colon cancer prognosis disclosed supra. As described herein, the expression levels of genes informative of colon cancer progression can be detected using the Affymetrix U133 gene expression arrays following the manufacturer's protocols. In a preferred embodiment of the present invention, however, the microarray comprises a plurality of nucleic acid probes, each nucleic acid probe having a nucleotide sequence that is complementary to at least a portion of a nucleotide sequence (RNA or DNA) of a gene selected from the group of 176 genes informative of colon cancer outcome disclosed supra. In another embodiment, the microarray comprises a plurality of nucleic acid probes, each nucleic acid probe having a nucleotide sequence that is complementary to at least a portion of a nucleotide sequence (RNA or DNA) of a gene selected from the group of 71 genes informative of colon cancer outcome described supra. In accordance with this aspect of the present invention, the nucleic acid probes of the present invention have a nucleotide sequence that is complementary to at least a portion of an RNA transcript or DNA nucleotide sequence encoded by a gene informative of colon cancer outcome. Exemplary nucleic acid probes having nucleotide sequences complementary to the RNA transcripts encoded by the 176 genes and the 71 genes informative of colon cancer outcome are provided in Table 1 by their Affymetrix identifier.

In another embodiment of the present invention, the microarray comprises a plurality of nucleic acid probes, each nucleic acid probe having a nucleotide sequence that is complementary to at least a portion of a nucleotide sequence (i.e., RNA transcript or DNA nucleotide sequence) of a gene selected from the group of 101 genes informative of colon cancer outcome disclosed supra. Exemplary nucleic acid probes having nucleotide sequences complementary to the RNA transcripts encoding the 101 genes informative of colon cancer outcome are provided in Table 2 by their Affymetrix identifier.

In another embodiment of the present invention, one or more supplementary analyses is performed to supplement or confirm the prognosis prediction achieved with the gene expression level analysis. In accordance with this embodiment of the present invention, the one or more additional analyses includes detecting microsatellite instability, measuring DNA promoter methylation, screening one or more mutations in one or more colon cancer oncogenes or tumor suppressor genes in the sample, or any combination of these analyses. The prognosis of a subject having colon cancer is then based on the detected expression levels of genes known to be informative of colon cancer in combination with one or more of these independent, additional analysis.

A deficient DNA mismatch repair (MMR) system is observed in about 10-15% of all colorectal carcinomas and in up to 90% of hereditary non-polyposis colorectal cancer (HNPCC) patients. Tumors with MMR defects acquire mutations in short repetitive DNA stretches, a phenomenon termed microsatellite instability. Therefore, the determination of microsatellite status is an ideal independent confirmatory prognostic analysis to perform in accordance with the methods of the present invention. Additionally, because the efficacy of adjuvant chemotherapy can be dependent on the microsatellite status of the tumor, determining the microsatellite status can be particular relevant to determining an effective individualized treatment plan for a subject having colorectal cancer.

In accordance with this aspect of the present invention, a favorable prognosis exists when a microsatellite instability-low status is detected, whereas an unfavorable prognosis exists when a microsatellite instability-high status is detected.

Methods and techniques for detecting microsatellite instability in a sample are well known in the art and are suitable for use in accordance with this aspect of the invention. In a preferred embodiment, microsatellite instability detection is performed using a PCR-based method to amplify tumor DNA and detect the five microsatellite markers established by the National Cancer Institute (Boland et al., “A National Cancer Institute Workshop of Microsatellite Instability for Cancer Detection and Familial Predisposition: Development of International Criteria for the Determination of Microsatellite Instability in Colorectal Cancer,” Cancer Res. 58(22):5248-57 (1998), which is hereby incorporated by reference in its entirety). These five microsatellite markers include two mononucleotide repeats (BAT26 and BAT25) and three dinucleotide repeats (D2S123, D5S346, and D17S250). The multiplex assay for rapid and accurate detection of the NCI 5-marker panel described by Nash et al., “Automated, Multiplex Assay for High-Frequency Microsatellite Instability in Colorectal Cancer,” J. Clin. Oncol. 21:3105-12 (2003), which is hereby incorporated by reference in its entirety, is particularly well suited for use in accordance with this aspect of the present invention. Alternatively, a PCR-based method for assessing the microsatellite instability status of a sample can be employed (e.g. detection of the 3′ UTR mononucleotide repeat, T25 (CAT25), of the CASP2 gene as described in U.S. Patent Application Publication No. 20080096197 to Findeisen et al., which is hereby incorporated by reference in its entirety).

Immunohistochemical approaches for detecting microsatellite instability are also suitable for use in accordance with this aspect of the present invention. Monoclonal antibodies specific for DNA mismatch repair genes, for example MLH1, MSH2, MSH6, and PMS2 have been described by Marcus et al. “Immunohistochemistry for hMLH1 and hMSH2: A Practical Test for DNA Mismatch Repair-Deficient Tumors,” Am J Surg Pathol. 23(10):1248-55 (1999); Lindor et al. “Immunohistochemistry Versus Microsatellite Instability Testing in Phenotyping Colorectal Tumors,” J Clin Oncol. 20(4):897-9 (2002); and Umar et al. “Revised Bethesda Guidelines for Hereditary Nonpolyposis Colorectal Cancer (Lynch syndrome) and Microsatellite Instability,” J Natl Cancer Inst. 96 (4):261-8 (2004), which are hereby incorporated by reference in their entirety.

A second analysis that is suitable to complement the detection of gene expression levels involves measuring the level of DNA promoter methylation. In higher order eukaryotic organisms, DNA methylation occurs at cytosines located 5′ to guanosine in a CpG dinucleotide. This modification has important regulatory effects on gene expression predominantly when it involves CpG rich areas known as CpG islands that are located in the promoter region of a gene sequence. Extensive methylation of CpG islands in tumor-suppressor genes has been associated with reduced expression of the tumor suppressor gene, resulting in unchecked cellular growth, tissue invasion, angiogenesis, and metastases. For example, the aberrant methylation of the Mut L homologue 1 gene (hMLH1) resulting in defective DNA mismatch repair has been associated with colorectal cancer. In accordance with this aspect of the invention, hMLH1 promoter methylation can be measured to compliment or confirm the gene expression detection analysis. Other genes known to be hypermethylated in colon cancer which are also suitable for promoter methylation analysis in accordance with this aspect of the invention include HPP1 (Sato et al., “Aberrent Methylation of the HPP1 Gene in Ulcerative Colitis-Associated Colorectal Carcinoma,” Cancer Research 62:6820-22 (2002), which is hereby incorporated by reference in its entirety); Reprimo (Takahashi et al., “Aberrent Methylation of Reprimo in Human Malignancies,” Int J Cancer 115(4):503-10 (2005), which is hereby incorporated by reference in its entirety); NEURL and FOXL2 (Schuebel et al., “Comparing the DNA Hypermethylome with Gene Mutations in Human Colorectal Cancer,” PLOS Genet. 3(9):e157-(2007), which is hereby incorporated by reference in its entirety); and ADAMTS1, CRABP1, and NR3C1 (Lind et al., “ADAMTS1, CRABP1, and NR3C1 identified as Epigenetically Deregulated Genes in Colorectal Tumorigenesis,” Cell Oncology 28(5-6):259-72 (2006), which is hereby incorporated by reference in its entirety).

In a preferred embodiment of the present invention the methylation level of the lecithin:retinol acyl transferase (LRAT) gene promoter nucleotide sequence, or region upstream thereof, is measured (See U.S. Patent Application Publication No. US20050227265 to Barany et al. and WO2008/077095 to Barany et al., which are hereby incorporated by reference in their entirety). In accordance with this aspect of the invention, a favorable prognosis exists when an increase in the methylation level of the lecithin:retinol acyl transferase gene promoter nucleotide sequence, or region upstream thereof, is measured.

DNA promoter methylation can be measured at a genome-wide or gene-specific level. For global methylation analysis, chromatographic methods, such as reverse-phase high pressure liquid chromatography and methyl accepting capacity assays are generally used. Alternatively, the restriction landmark genomic scanning for methylation (RLGS-M) assay as described by Hayashizaki et al., “Restriction Landmark Genomic Scanning Method and its Various Applications,” Electrophoresis 14(4):251-8 (1993) and CpG island microarry can also be used to measure genome-wide methylation. Various techniques available to measure gene-specific methylation, include DNA digestion with a methylation sensitive restriction enzyme followed by Southern blot detection of PCR amplification; methylation specific PCR; bisulfite genomic sequencing PCR; or in situ immunodetection using 5-methylcytosine specific antibody as described by Castilho et al., “5-Methylcytosine Distribution and Genome Organization in Triticale Before and After Treatment with 5-Azacytidine,” J Cell Sci 112:4397-404 (1999), which is hereby incorporated by reference in its entirety). Additional methods and techniques for measuring DNA methylation including the nearest neighbor analysis, chemical DNA sequencing, methylation sensitive restriction fingerprinting, combined bisulfite restriction analysis, and methyl-CpG binding column isolation are described in DNA Methylation Protocols (Mills and Ramsahoye, eds., Humana Press 2002), which is hereby incorporated by reference in its entirety. In a preferred embodiment, DNA promoter methylation analysis is carried out using the quantitative bisulfite-PCR/LDR/Universal Array platform described in U.S. Patent Application Publication No. US20050227265 to Barany et al.; WO2008/077095 to Barany et al.; and Chen et al., “Multiplexed Profiling of Candidate Genes for CpG Island Methylation Status using a Flexible PCR/LDR/Universal Array Assay,” Genome Research 16:282-9 (2006) which are incorporated by reference in their entirety.

In another embodiment of the present invention, the mutational status of one or more colon cancer oncogenes or tumor-suppressor genes is screened. The presence or absence of such mutations can contribute to the determination of a subject's prognosis. Mutations in several such genes, especially DNA mismatch repair genes, are well known in the art and can be screened in accordance with this aspect of the invention. In a preferred embodiment, the mutational status of K-ras, B-raf, APC, p53, PIK3CA, is screened. An unfavorable prognosis exists when mutations in one or more of these colon cancer oncogenes or tumor suppressor genes is identified.

Any art acceptable method for detecting the mutational status of a gene can be used in accordance with this aspect of the invention. Preferred methods include the endonuclease/ligase based mutation scanning method (Huang et al., “An Endonuclease/Ligase Based Mutation Scanning Method Especially Suited for Analysis of Neoplastic Tissue,” Oncogene 21:1909-21 (2002) and U.S. Pat. No. 7,198,894 to Barany et al., which are hereby incorporated by reference in their entirety); ligase detection reaction (LDR) (U.S. Pat. No. 6,312,892 to Barany et al., which is hereby incorporated by reference in their entirety); coupled LDR/PCR (U.S. Pat. Nos. 7,097,980, 6,797,470, 6,268,148, and 6,027,889 all to Barany et al., which are hereby incorporated by reference in their entirety); coupled PCR/restriction endonuclease digestion/LDR reaction (U.S. Pat. No. 7,014,994 to Barany et al., which is hereby incorporated by reference in its entirety); ligase detection reactions using addressable arrays (U.S. Pat. No. 7,083,917 to Barany and U.S. Patent Application Publication Nos. 20020150921, 20030022182, 20040259141, and 20040253625 all to Barany et al., which are hereby incorporated by reference in their entirety) and DNA microarray multiplex detection methods (Gerry et al., “Universal DNA Microarray Method for Multiplex Detection of Low Abundant DNA Mutations,” J Mol Biol 292:251-62 (1999), which is hereby incorporated by reference in its entirety). Other suitable methods for determining the mutational status of a gene include direct DNA sequencing techniques, (e.g. Sanger dideoxy or Maxam-Gilbert sequencing reactions) and massively parallel sequencing technology.

In a preferred embodiment of the present invention, the data generated from the detection of gene expression levels of the at least five genes selected from the group of 176, 71, or 101 genes informative of colon cancer prognosis is used to prepare a personalized genomic profile for a colon cancer patient. Information regarding microsatellite instability, DNA promoter methylation, and the mutational status of one or more oncogenes or tumor-suppressor genes can also be incorporated into an individual's personalized genomic profile. The genomic profile can be used to establish a personalized treatment plan for the colon cancer patient. Such treatment plan may consist of surgery, individual therapy, chemotherapy, radiation therapy or any combination thereof. In accordance with this aspect of the invention, the colon cancer patient is administered a cancer treatment based on the treatment plan.

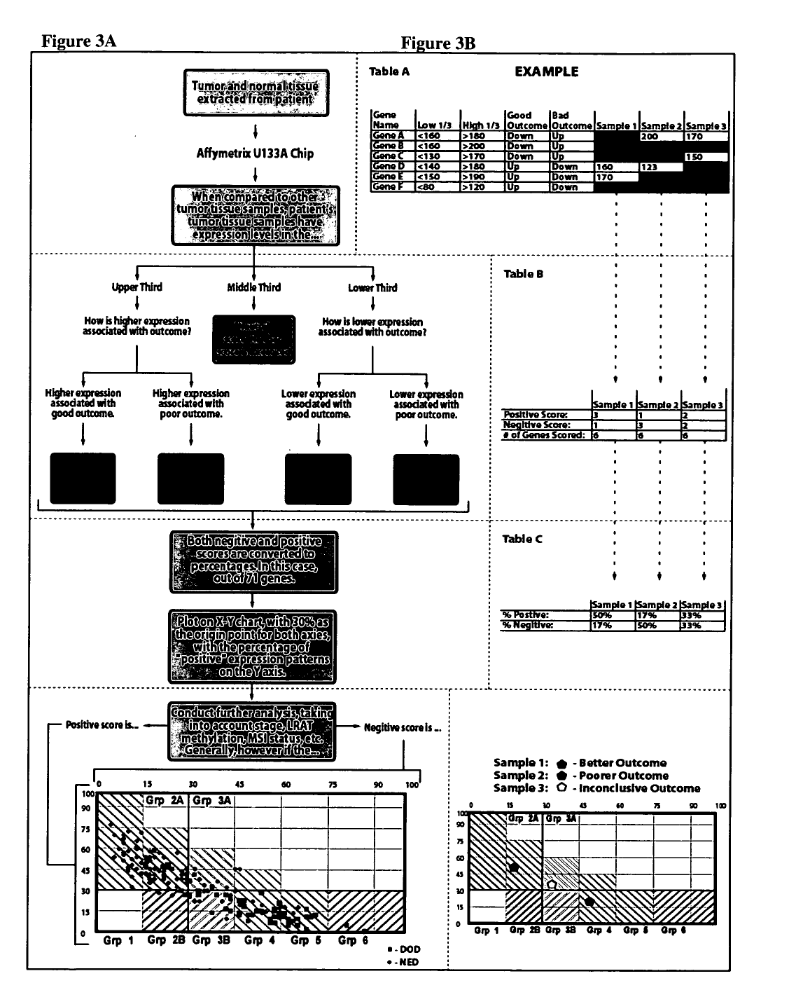

FIG. 3 summarizes how a colon cancer patient's prognosis is determined using the 71, 101, or 176 gene predictor sets of the present invention. The left side of the figure outlines the steps involved in identifying genes predictive of colon cancer outcome generally, while the right side of the figure outlines the method of determining the prognosis of a subject having colon cancer of the present invention using three hypothetical patient samples where the expression of six genes is analyzed. First, the gene expression levels of at least five, but preferably all of the 71, 101, or 176 genes in a tumor sample obtained from the patient are determined and compared to average tumor sample expression levels. If gene expression for a particular gene is in the upper third of average tumor expression level in the patient sample and higher expression of that gene is associated with a bad disease expression profile, the patient is given a negative mark or negative score (see FIG. 3A). If, however, higher gene expression is associated with a good disease outcome, the patient is given a positive mark or score. As shown in the hypothetical example (FIG. 3B), the expression levels for genes A-F were assessed in samples 1-3. In sample 1, Genes A and C had expression values in the lower third of average tumor expression levels (see FIG. 3B, Table A, compare values in column 5 with values in column 2). Low expression of Genes A and C are associated with a good outcome (see FIG. 3B, Table A, column 4). Accordingly, sample 1 was given positive scores for these genes as indicated by the blue shading. Also in sample 1, Genes B and F had expression levels in the top third of average tumor expression levels. High expression of Gene B is associated with a bad outcome (sample 1 given negative score indicated by red shading), while high expression of Gene F is associated with a good outcome (blue shading). In total for sample 1, the expression levels of three genes was associated with a good disease outcome (i.e. Genes A, C, and F, FIG. 3B, Table B) resulting in a positive score of 3, while the expression level of one gene was associated with a bad disease outcome (i.e. Gene B) resulting in a negative score of 1 (genes E and F had neutral scores).

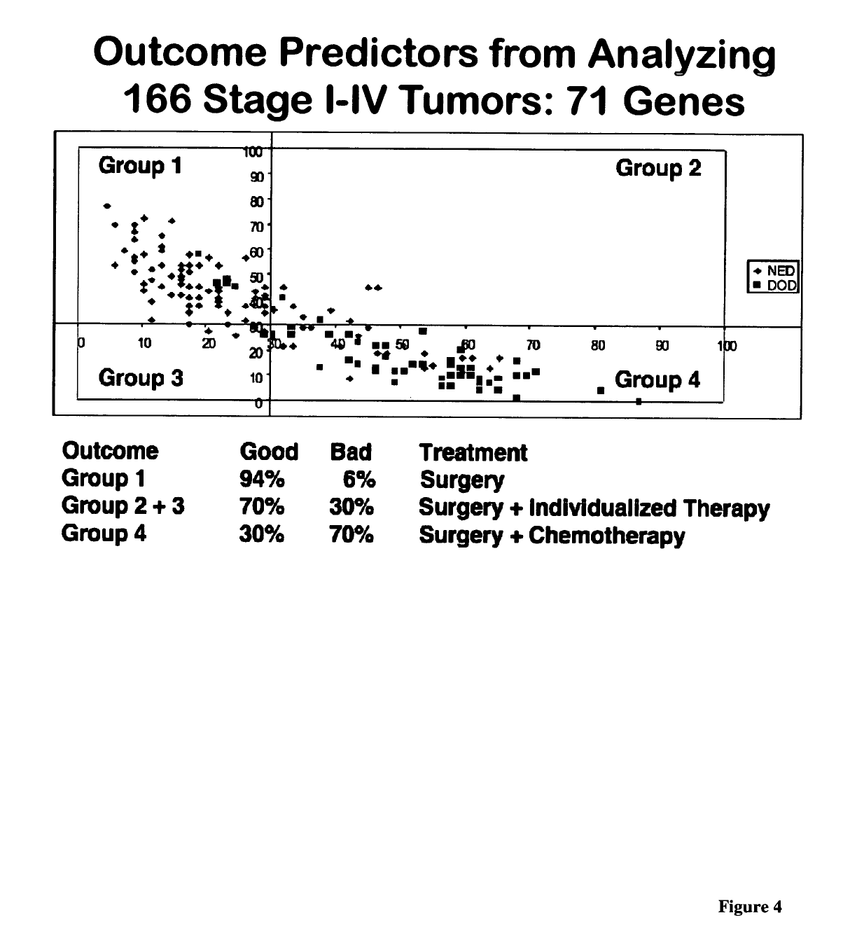

The negative and positive scores are converted to percentages based on the total number of genes analyzed. In the hypothetical example, sample 1 had 3 out of 6 genes, or 50%, with favorable or positive expression levels, and 1 out of 6 genes, or 17% with unfavorable or negative expression levels (FIG. 3B, Table C). The predicted outcome for the patient is determined by plotting the percentage of genes in the tumor sample that had expression values associated with a good disease outcome (y-axis) versus the percentage of genes in the tumor sample having expression levels associated with a bad disease outcome (x-axis) where the point of origin is set to 30%. In the hypothetical example, sample 1, with 50% of genes having expression levels associated with a good outcome and 17% of genes having expression levels associated with bad outcome falls into Group 2A, where the prognosis is generally favorable (FIG. 4B, scatterplot). Sample 2, with 17% of the genes having expression levels associated with a good outcome and 50% of the genes having expression levels associated with bad outcome falls into Group 4, where the prognosis is generally unfavorable. Sample 3, having 33% of the gene analyzed having expression levels associated a good outcome and 33% associated with a bad disease outcome binned to Group 3A, where the prognisis is generally inconclusive.

As indicated in FIG. 3A, supplementary analyses (i.e. LRAT methylation, MSI status, etc.) can be performed to provide additional prognostic information for patients that fall into intermediate groups (i.e. Groups 2 and 3) or to confirm the prognosis of those patients in Group 1.

As discussed supra, the predicted outcome for a patient, determined by gene expression levels as outlined above, can be used to guide treatment. For example, patients who bin to Group 1 have a favorable prognosis and may benefit from surgery only, whereas patients who bin to Group 4 have an unfavorable prognosis and may need to supplement surgery with chemotherapy or other more aggressive therapies. Treatment decisions should further take into consideration the stage of the tumor. For example, individuals with stage 2 tumors in Group 1 or 2A will most likely benefit from surgery without additional treatment. Individuals with stage 3 tumors in these groups are probably responsive to standard care. Individuals with stage 3 tumors in Groups 4 and 5 will most likely not be responsive to standard care, and thus would be candidates for enrolling into clinical trials of novel therapies.

The present invention is also directed to a method of identifying an agent that improves the prognosis of a subject having colon cancer. This method involves administering an agent (i.e., a candidate agent) to the subject having colon cancer and obtaining a first biological sample from the subject before said administering and a second biological sample from the subject after said administering. The method further involves detecting the expression level of at least five genes selected from the group of 176 genes informative of colon cancer prognosis disclosed supra. Determining increases or decreases in the expression levels of the at least five genes in the second sample compared to the first sample identifies an agent that improves the prognosis of a subject having colon cancer. In a preferred embodiment of this aspect of the present invention, the at least five genes is selected from the group of 71 genes informative of colon cancer prognosis disclosed supra.

In accordance with this aspect of the present invention, an agent that increases the expression levels of any one of the following genes: SERPINA1, RPLP0, RPLP0-like, CYB561, AKR1A1, AP3D1, ARL6IP4, OGFOD2, ASNA1, CFB, ERP29, SMG7, CASP1, CCNA2, LOC100131861, SAMM50, COX5A, CXCL11, DAZAP2, DDX23, FDFT1, COMMD4, GCHFR, GRHPR, GSR, ISG20, ITGAE, KIAA0746, SERINC2, FRYL, LRRC47, LAMP3, R3HCC1, MAPKAPK5, MCM5, MCRS1, TMEM106C, MMP3, MTUS1, LRRC41, NAT1, NDUFC1, YBX1, PEBP1, PIGR, PSMA5, SFPQ, SLC25A3, SLC39A8, SQRDL, SRP72, SSNA1, TAPBPL, TEGT, PBK, UCP2, UQCRH, XPO7, CCT2, CNOT7, DHX15, TMEM87A, ELP3, FAM82C, LL22NC03-5H6.5, DENND2D, WDR68, IL15RA, DENND2A, KIF13B, MFHAS1, SPATA5L1, MYRIP, PIGT, PSMD9, RPS27L, TNFRSF10B, UBE2L6, USP3, ATP5B, CALML4, C1orf144, TMEM33, C12orf52, GHITM, H2AFZ, LAP3, MRPL46, SORD, CNPY2, TNFSF10, U2AF2, CMPK1, UQCRFS1, WARS and WDR1 is an agent that improves the prognosis of a subject having colon cancer. An agent that causes a decrease in the expression levels of any one of the following genes: AK023058*, AIP, ARL2BP, C1GALT1, CDC42BPA, C8orf70, CLN5, COBL, CYB5B, MOSPD1, DOCK9, EGFR, FKBP14, DND1, DND1, GREM2, GPR177, GALNS, GRB10, GRP, GSTA1, RP3-377H14.5, HOXB7, ZNF117, TNIK, LANCL1, METRN, LEPREL1, NAB1, NISCH, OGT, OSBPL3, PDGFA, PRDM2, PRELP, PSPC1, RECQL, RYK, SMURF2, TLN1, UNC84A, USP12, ZMYM2, ZMYM5, AL359599*, ARL4A, N4BP2L2, GLS, C19orf36, TMCC1, METTL3, TMEM16A; RTN2, SCAMP1, SF3B1, SOX4, STK3, ZNF430, C6orf15, C7orf10, CHST12, ETV1, ACSL4, FLJ10357, C5orf23, AA058828*, CDR2L, KLC1, MAP4, NUMB, PAM, PGDS, PTHLH, ZC3H7B, SAV1, SGCD, SYNGR1, TES, IFT88, TRIM36 and VPS41 is an agent that improves the prognosis of a subject having colon cancer.

Another aspect of the present invention is directed to a collection of 71 genes having expression levels informative for predicting a prognosis of a patient having colon cancer. This collection of 71 genes includes the following genes of Table 1: SLC25A3, DAZAP2, TEGT, ERP29, PSMA5, DDX23, LOC100131861, SAMM50, SFPQ, NISCH, CYB5B, TMEM106C, EGFR, MCRS1, SERPINA1, CCNA2, NDUFC1, COX5A, GCHFR, ITGAE, PRDM2, PDGFA, GSR, GRP, COMMD4, XPO7, YBX1, SRP72, UCP2, SLC39A8, NAB1, WDR68, CXCL11, RECQL, CASP1, PTHLH, UNC84A, MTUS1, KIAA0746, SERINC2, DOCK9, FRYL, MAPKAPK5, LRRC47, RQCD1, TNIK, RPLP0, RPLP0-like, CLN5, NAT1, CDC42BPA, GSTA1, ZMYM5, RYK, PIGT, CMPK1, SQRDL, FAM82C, CNOT7, LL22NC03-5H6.5, PSPC1, TAPBPL, METRN, PBK, MRPL46, FKBP14, C1GALT1, GREM2, GPR177, DND1, and PRELP. The collection of 71 genes informative of predicting the prognosis of a patient having colon cancer can further include the following genes of Table 1: AA058828*, ACSL4, AIP, AK023058*, AKR1A1, AL359599*, AP3D1, ARL2BP, ARL4A, ARL61P4, OGFOD2, ASNA1, ATP5B, C12orf52, C19orf36, C1orf144, C5orf23, C6orf15, C7orf10, C8orf70, CALML4, CCT2, CDR2L, CFB, CHST12, CNPY2, COBL, CYB561, DENND2A, DENND2D, DHX15, DND1, ELP3, ETV1, FDFT1, FLJ10357, GALNS, GHITM, GLS, GRB10, GRHPR, H2AFZ, HOXB7, IFT88, IL15RA, ISG20, KIAA0746, SERINC2, KIF13B, KLC1, LAMP3, LANCL1, LAP3, LEPREL1, LRRC41, MAP4, MCM5, METTL3, MFHAS1, MMP3, MOSPD1, MYRIP, N4BP2L2, NUMB, OGT, OOSBPL3, PAM, PEBP1, PGDS, PIGR, PSMD9, R3HCC1, RP3-377H14.5, RPS27L, RTN2, SAV1, SCAMP1, SF3B1, SGCD, SLC39A8, SMG7, SMURF2, SORD, SOX4, SPATA5L1, SSNA1, STK3, SYNGR1, TEGT, TES, TLN1, TMCC1, TMEM16A, TMEM33, TMEM87A, TNFRSF10B, TNFSF10, TRIM36, U2AF2, UBE2L6, UCP2, UQCRFS1, UQCRH, USP12, USP3, VPS41, WARS, WDR1, ZC3H7B, ZMYM2, ZNF117, and ZNF430.

Another aspect of the present invention is related to a collection of 101 genes having expression levels informative for predicting a prognosis of a patient having colon cancer. The collection of 101 genes are provided in Table 2 above.

Also included in the present invention are arrays that are useful for practicing one or more of the above described methods. Such arrays consist of nucleic acid or peptide-based probes that are useful for detecting the expression of one or more genes, preferably at least five genes, from the collection of 71, 101, or 176 genes that are informative for predicting the prognosis of a subject having colon cancer, using any of the methods described supra for detecting gene expression. A variety of different array formats are known in the art with a wide variety of probe structures, substrate compositions, and attachment technologies (See e.g. U.S. Pat. Nos. 5,143,854 to Pirrung et al.; 5,288,644 to Beavis et al.; 5,324,633 to Fodor et al.; 5,432,049 to Fischer et al.; 5,470,710 to Weiss et al.; 5,492,806 to Drmanac et al.; 5,445,934 to Fodor et al.; 5,744,305 to Fodor et al.; 5,677,195 to Winkler et al.; 6,040,193 to Winkler et al.; and 5,424,186 to Fodor et al., which are all hereby incorporated by reference in their entirety). In a preferred embodiment, array(s) of the present invention consist of a plurality of nucleic acid probes, each nucleic acid probe having a nucleotide sequence that is complementary to at least a portion of a nucleotide sequence (e.g., RNA or DNA) of a gene selected from the collection of 71 genes, 101 genes, 176 genes, or any combination thereof. Exemplary nucleic acid probes having nucleotide sequences complementary to at least a portion of the nucleotide sequences (i.e., RNA transcript) encoded by the genes of the 71, 101, and 176 gene collections are provided in Tables 1 and 2, although variations of those probes, or other probes may also be suitable for use.

In a preferred embodiment of the present invention the arrays of the present invention are available together with suitable reagents as a kit. The kit can be used to determine gene expression levels in biological sample(s) from a subject having colon cancer and determine his or her prognosis. Additional reagents suitable for inclusion in such kits include, but are not limited to, gene specific primers for the collections of the 71, 101, and/or 176 genes, universal primers, dNTPs and/or rNTPS, fluorescent, biotinylated, or other post-synthesis labeling reagents, enzymes such as reverse transcriptase, DNA and/or RNA polymerases, and various wash and buffer mediums.

Another aspect of the present invention relates to a method for determining a subject's predisposition to having colon cancer. This method involves obtaining a biological sample from the subject and detecting the expression levels of at least five gene selected from the collection of 176 genes informative of colon cancer predisposition disclosed supra. The method further involves comparing the detected expression levels of the at least five genes from said sample with the expression levels of the corresponding five genes associated with a having a predisposition to colon cancer and determining the subject's predisposition to having colon cancer based on said comparing.

### EXAMPLES

### Example 1

### Biological Sample Description and Collection

Expression array data was generated from 183 primary colon cancer (PCC) tumors, 46 large adenomas, 39 liver metastasis, 19 lung metastasis, 53 normal mucosa, 7 normal lung, and 12 normal liver tissues. In addition, SNP array data was collected from 89 colorectal (CRC) tissue samples (65 primary colon cancer, 9 liver metastasis, 10 lung metastasis, and 5 unclassified colon cancer), as well as 56 normal tissues (i.e., normal mucosa, liver, or kidney), 51 of which were matched to the CRC tissues. Tissue samples were obtained from CRC patients at Memorial Sloan Kettering Cancer Center (MSKCC), whose initial operations occurred between 1992 and 2004. Cancer samples included in SNP array analysis were characterized by pathologists (MSKCC) to have ≧70% pure tumor cells. Acquisition of tissues followed the strict protocols of the Institutional Review Boards of MSKCC and Cornell University Weill Medical College.

### Example 2

### RNA Preparation

Total RNA from microdissected tissue samples (both tumor and normal tissue samples) was prepared following the protocol recommended by Affymetrix (Santa Clara, Calif.). RNA was extracted from homogenized tissues using the Trizol protocol (Guanidinium thiocyanate-phenol-chloroform extraction) (Invitrogen Corp.) and purified using RNeasy columns (Qiagen).

### Example 3

### Genomic DNA Sample Extraction

Microdissected tissue samples (50-100 mg) were homogenized in liquid nitrogen and suspended in 400 ul proteinase K solution (50 ul 20 mg/ml proteinase K in proteinase K buffer). Phenol/chloroform (500 ul) was added and the mixture was shaken thoroughly in a phase lock gel tube. The upper aqueous layer containing genomic DNA was transferred to a separate tube and washed with isopropanol and 70% ethanol. The resulting pellet was resuspended in molecular biology-grade water.

### Example 4

### Expression Array

To generate the expression array data, the protocol recommended by Affymetrix, Inc. was strictly followed. Briefly, first strand cDNA was synthesized from 10 mg total RNA, using the One-Cycle cDNA Synthesis kit (which includes T7 (dT) primer, and SuperScript II Reverse Transcriptase). Additional reagents from the same kit (i.e., 2nd strand reaction mix, E. coli DNA ligase, and E. coli Polymerase I) were used to synthesize the 2nd strand cDNA. The cDNA product was transcribed in vitro to produce biotin-labeled cRNA, using MEGAscript T7 Kit (Ambion, Inc.). The labeled cRNA was fragmented and hybridized to GeneChip Human Genome U133A Array chip at 45° C. for 16 h. Afterwards, the arrays were washed and stained using SAPE (streptavidin-phycoerythrin) and biotinylated anti-streptavidin antibody. All of the washing and staining procedures were conducted using the Affymetrix Fluidic Station 450 (FS450). Following hybridization, the arrays were scanned using the GeneChip Scanner 3000. The Affymetrix GCOS software was used to generate image (DAT), cell intensity (CEL), and analysis (CHP) files for every sample. Standard thresholding, filtering operations, and normalizations were applied such that the average intensity value across all probesets for every sample was around 69.

### Example 5

### Kaplan-Meier Survival Analysis

The primary colorectal cancer samples were classified into two groups according to the level of gene expression as determined by the Affymetrix U133A expression array. Kaplan-Meier survival analysis was used to determine the disease-specific survival patterns on selected genes in areas of chromosomal aberrations. Follow-up (0-175 months; median 74 months) was censored at death from other causes for the Kaplan-Meier analysis. Statistical analysis and curves were generated using the JMP statistical software (version 5.1.2, SAS institute, Cary, N.C., USA).

### Example 6

### Identifying Genes That Predict Disease Outcome in Patients Having Colon Cancer

Primary colon tumor samples from 166 patients were used in the analysis to identify genes that are predictive of disease outcome. Of these samples, 56 were derived from patients that had died of disease (DOD), and 110 samples were derived from patient that either had no evidence of disease (NED) in long term follow up, were alive with disease (AWD), or died of other or unknown causes (DOC/DUC). Samples from the 110 patients who did not die of disease are collectively referred to as “non-DOD”.

FIG. 2 depicts the steps of identifying the 176 and 71 gene predictor sets of the present invention that are useful for predicting disease outcome in subjects having colon cancer. First, the expression levels of 22283 gene transcripts in the 166 primary colon cancer samples were analyzed and classified as having high, average, or low expression based on percentile ranks. An initial score was generated for gene expression in each sample wherein +1 was assigned for higher than average tumor expression and 0 for lower than average expression. A second score was also generated wherein +1 was assigned for expression levels in the top third of average tumor expression levels, 0 was assigned for expression levels in the middle third of average tumor expression levels, and −1 was assigned for expression levels in the bottom third of average tumor expression levels. Genes that had poor expression patterns as determined by the average expression level and the standard deviation, or genes that had expression patterns that did not differ significantly from normal samples were eliminated from the analysis (FIG. 2).

A computer analysis was performed to identify genes that had expression levels in the top third in samples from patients who died of disease (DOD) but in the bottom third in samples taken from patients who did not die of disease (non-DOD), and identify genes that had expression levels in the bottom third in samples from DOD patients, but in the top third in samples from non-DOD patients. This analysis identified genes that had different expression patterns in DOD and non-DOD samples and were candidates for further analysis.

A difference score for each of these candidate gene was then calculated by subtracting the total number of DOD tumor samples where gene expression was in the bottom third of tumor expression from the total number of DOD tumor samples where gene expression was in the top third of tumor expression. Genes having a difference score outside of 12 to 19 or -23 to −12 were eliminated from analysis while the remaining genes, 383 in total, were further analyzed using Kaplan-Meier survival curves (FIG. 2).

Kaplan-Maier curves were manually generated for all of the 383 genes using the JMP statistical analysis program (SAS Institute, Cary, N.C.). The chi-square values and p-values for all of these curves were then used to sort the genes by the greatest difference in survival based on expression. The 383 gene set that was identified based on difference scores was narrowed to 176 genes, where the 176 genes had KM curves with a p-value ≦0.02. The 176 gene set was further narrowed to 71 genes based on those genes having KM curves with a p-value of ≦0.0125 as shown in FIG. 2.

Table 3 below summarizes additional parameters calculated for each gene in the 176 gene set, which includes the 71 gene set. These parameters include (1) the average expression value for a particular gene across all tumor samples (“Ave Tumor”) and the standard deviation for expression for each gene probe used to detect expression (“Stdev Tumor”); (2) the difference score (“Diff”) which is the total number of DOD samples where the gene expression level was in the top third of tumor expression level minus the total number DOD samples where the gene expression level was in the bottom third of tumor expression level; (3) the percentage DOD samples having gene expression values in the top third of tumor expression (“D+1%”); (4) the percentage of DOD samples having gene expression values equal to the average, or the middle third of tumor expression (“D0%”); (5) the percentage of DOD samples having gene expression values in the bottom third of tumor expression (“D−1%”); (6) the percentage of difference between the two curves in the Kaplan-Meier analysis (“KM %”) calculated by dividing the number of DOD samples where the gene was expressed in the top third over the number of DOD and non-DOD samples where the gene was expressed in the top third; and 7) the chi-square and p-values of the KM survival curve analysis. The last two columns of Table 3 indicate whether increased (“up”) or decreased (“down”) expression of the particular gene predicts an unfavorable prognosis (“Bad Outcome Score”) or a favorable prognosis (“Good Outcome Score”).

TABLE 3
Gene Expression Data for the 176-Gene Predictor Set

| | | | | | | | | | | | D + 1 U1Bad | D − 1 Good |
|---|---|---|---|---|---|---|---|---|---|---|---|---|
| | Accession | Avg | Stdev | | KM | | | | CHI | | Outcome | Outcome |
| Gene Symbol | Number | Tumor | Tumor | Diff | % | D + 1% | D0% | D − 1% | Square | P value | Score | Score |
| CYB5-M | 80777 | 155 | 75 | 19 | 35 | 53 | 31 | 18 | 12.1 | 0.0023 | Up | down |
| PSPC1 | 55269 | 128 | 40 | 19 | 35 | 53 | 31 | 18 | 11.8 | 0.0027 | Up | down |
| UNC84A | 23353 | 404 | 130 | 19 | 35 | 51 | 35 | 16 | 11.7 | 0.0029 | Up | down |
| CDC42BPA | 8476 | 186 | 84 | 19 | 35 | 53 | 31 | 18 | 11.6 | 0.0030 | Up | down |
| FLJ23091 | 79971 | 139 | 55 | 18 | 33 | 56 | 22 | 23 | 17.3 | 0.0002 | Up | down |
| CIGALT1 | 56913 | 96 | 36 | 18 | 33 | 56 | 22 | 23 | 15.6 | 0.0004 | Up | down |
| RYK | 6259 | 101 | 32 | 18 | 33 | 51 | 33 | 18 | 12.1 | 0.0024 | Up | down |
| MGC2601 | 79006 | 76 | 45 | 18 | 33 | 55 | 25 | 21 | 11.2 | 0.0037 | Up | down |
| PRDM2 | 7799 | 127 | 30 | 18 | 33 | 51 | 33 | 18 | 9.9 | 0.0072 | Up | down |
| KIAA0551 | 23043 | 108 | 48 | 18 | 33 | 47 | 40 | 14 | 9.4 | 0.0090 | Up | down |
| FKBP14 | 55033 | 74 | 30 | 18 | 33 | 51 | 33 | 18 | 9.3 | 0.0091 | Up | down |
| GSTA1 | 2938 | 89 | 22 | 18 | 33 | 49 | 37 | 16 | 8.9 | 0.0115 | Up | down |
| FLJ21195 | 64388 | 29 | 22 | 17 | 31 | 53 | 27 | 21 | 15.6 | 0.0004 | Up | down |
| EGFR | 1956 | 135 | 55 | 17 | 31 | 55 | 24 | 23 | 15.2 | 0.0005 | Up | down |
| NAB1 | 4664 | 276 | 96 | 17 | 31 | 51 | 31 | 20 | 12.7 | 0.0017 | Up | down |
| ZNF237 | 9205 | 52 | 33 | 17 | 32 | 52 | 30 | 20 | 11.9 | 0.0026 | Up | down |
| DOCK9 | 23348 | 185 | 418 | 17 | 32 | 48 | 38 | 16 | 11.1 | 0.0039 | Up | down |
| PRELP | 5549 | 127 | 68 | 17 | 32 | 51 | 31 | 19 | 9.9 | 0.0071 | Up | down |
| FLJ20195 | 373863 | 96 | 32 | 17 | 32 | 50 | 34 | 18 | 8.8 | 0.0124 | Up | down |
| FLJ20195 | 373863 | 121 | 31 | 17 | 32 | 48 | 38 | 16 | 7.9 | 0.0195 | Up | down |
| PDGFA | 5154 | 146 | 71 | 16 | 29 | 47 | 36 | 18 | 12.3 | 0.0021 | Up | down |
| GRP | 2922 | 60 | 59 | 16 | 29 | 49 | 33 | 20 | 12.1 | 0.0023 | Up | down |
| PTHLH | 5744 | 37 | 143 | 16 | 29 | 49 | 33 | 20 | 11.9 | 0.0026 | Up | |
| NISCH | 11188 | 443 | 555 | 16 | 29 | 47 | 36 | 18 | 9.7 | 0.0078 | Up | down |
| CLN5 | 1203 | 69 | 24 | 16 | 29 | 49 | 33 | 20 | 9.5 | 0.0086 | Up | down |
| RECQL | 5965 | 78 | 28 | 16 | 30 | 53 | 25 | 23 | 9.2 | 0.0101 | Up | down |
| USP12 | 219333 | 163 | 62 | 16 | 29 | 49 | 33 | 20 | 8.6 | 0.0136 | Up | down |
| DJ473B4 | 56180 | 177 | 68 | 16 | 29 | 49 | 33 | 20 | 8.5 | 0.0141 | Up | down |
| OGT | 8473 | 370 | 151 | 16 | 30 | 46 | 39 | 16 | 8.3 | 0.0157 | Up | down |
| OSBPL3 | 26031 | 95 | 39 | 16 | 29 | 49 | 33 | 20 | 8.0 | 0.0180 | Up | down |
| GALNS | 2588 | 127 | 37 | 16 | 29 | 49 | 33 | 20 | 8.0 | 0.0184 | Up | down |
| GLS | 2744 | 209 | 96 | 16 | 29 | 45 | 40 | 16 | 10.4 | 0.0054 | | down |
| ZNF198 | 7750 | 282 | 94 | 15 | 28 | 47 | 35 | 20 | 14.1 | 0.0009 | Up | down |
| HOXB7 | 3217 | 60 | 28 | 15 | 28 | 47 | 35 | 20 | 13.2 | 0.0014 | Up | down |
| COBL | 23242 | 172 | 73 | 15 | 28 | 47 | 35 | 20 | 11.8 | 0.0027 | Up | down |
| GRB10 | 2887 | 228 | 115 | 15 | 28 | 49 | 31 | 21 | 11.6 | 0.0030 | Up | down |
| PGDS | 27306 | 41 | 24 | 15 | 30 | 53 | 26 | 23 | 11.0 | 0.0041 | Up | |
| TLN1 | 7094 | 134 | 39 | 15 | 28 | 49 | 31 | 21 | 10.7 | 0.0046 | Up | down |
| AIP | 9049 | 353 | 81 | 15 | 29 | 50 | 30 | 21 | 10.5 | 0.0054 | Up | down |
| C7orf10 | 79783 | 21 | 24 | 15 | 28 | 47 | 35 | 20 | 10.3 | 0.0057 | Up | |
| MLAT4 | 55214 | 66 | 110 | 15 | 28 | 47 | 35 | 20 | 9.5 | 0.0088 | Up | down |
| SGCD | 6444 | 32 | 21 | 15 | 28 | 51 | 27 | 23 | 9.3 | 0.0094 | Up | |
| SMURF2 | 64750 | 196 | 82 | 15 | 28 | 47 | 35 | 20 | 9.2 | 0.0097 | Up | down |
| H-plk | 51351 | 108 | 44 | 15 | 28 | 49 | 31 | 21 | 9.1 | 0.0108 | Up | down |
| HLA-F | 285830 | 120 | 45 | 15 | 28 | 47 | 35 | 20 | 8.6 | 0.0134 | Up | down |
| — | | 68 | 33 | 15 | 28 | 47 | 35 | 20 | 8.5 | 0.0139 | Up | down |
| CGI-62 | 51101 | 132 | 58 | 15 | 29 | 50 | 30 | 21 | 8.2 | 0.0164 | Up | down |
| LANCL1 | 10314 | 120 | 42 | 15 | 28 | 47 | 35 | 20 | 8.2 | 0.0165 | Up | down |
| TRIM36 | 55521 | 36 | 24 | 15 | 28 | 47 | 35 | 20 | 7.8 | 0.0199 | Up | |
| RTN2 | 6253 | 90 | 45 | 15 | 28 | 45 | 38 | 18 | 10.4 | 0.0054 | | down |
| SOX4 | 6659 | 1055 | 392 | 15 | 28 | 44 | 42 | 16 | 9.5 | 0.0088 | | down |
| STK3 | 6788 | 136 | 70 | 15 | 28 | 45 | 38 | 18 | 8.6 | 0.0133 | | down |
| SCAMP1 | 9522 | 398 | 100 | 15 | 28 | 45 | 38 | 18 | 8.1 | 0.0177 | | down |
| KIAA0779 | 23023 | 125 | 45 | 15 | 28 | 45 | 38 | 18 | 8.0 | 0.0179 | | down |
| VPS41 | 27072 | 268 | 61 | 14 | 26 | 49 | 29 | 23 | 10.8 | 0.0044 | Up | |
| TES | 26136 | 561 | 170 | 14 | 26 | 51 | 25 | 25 | 10.5 | 0.0051 | Up | |
| CHST12 | 55501 | 63 | 31 | 14 | 26 | 49 | 29 | 23 | 10.5 | 0.0053 | Up | |
| BART1 | 23568 | 49 | 133 | 14 | 30 | 47 | 38 | 18 | 10.4 | 0.0054 | Up | down |
| PAM | 5066 | 446 | 158 | 14 | 26 | 51 | 25 | 25 | 10.4 | 0.0056 | Up | |
| KNS2 | 3831 | 267 | 113 | 14 | 26 | 49 | 29 | 23 | 10.0 | 0.0067 | Up | |
| C6orf15 | 29113 | 45 | 88 | 14 | 26 | 49 | 30 | 23 | 9.4 | 0.0089 | Up | |
| HUMPPA | 30850 | 64 | 86 | 14 | 26 | 49 | 29 | 23 | 9.0 | 0.0111 | Up | |
| FLJ14054 | 79614 | 63 | 50 | 14 | 26 | 51 | 26 | 25 | 8.6 | 0.0132 | Up | |
| RoXaN | 23264 | 102 | 44 | 14 | 26 | 49 | 30 | 23 | 7.9 | 0.185 | Up | |
| ZNF430 | 80264 | 122 | 43 | 14 | 26 | 42 | 44 | 16 | 10.2 | 0.0060 | | down |
| — | | 70 | 27 | 14 | 26 | 42 | 44 | 16 | 8.8 | 0.0120 | | down |
| ARL4 | 10124 | 285 | 111 | 14 | 26 | 45 | 36 | 20 | 8.8 | 0.0121 | | down |
| OGT | 8473 | 361 | 167 | 14 | 26 | 45 | 36 | 20 | 8.3 | 0.0158 | | down |
| SF3B1 | 23451 | 335 | 97 | 14 | 26 | 44 | 40 | 18 | 8.2 | 0.0163 | | down |
| SAV1 | 60485 | 80 | 43 | 13 | 24 | 51 | 24 | 27 | 12.1 | 0.0023 | Up | |
| FLJ10357 | 55701 | 115 | 50 | 13 | 24 | 51 | 24 | 27 | 10.6 | 0.0051 | Up | |
| ETV1 | 2115 | 37 | 32 | 13 | 24 | 51 | 24 | 27 | 10.0 | 0.0066 | Up | |
| FACL4 | 2182 | 58 | 31 | 13 | 24 | 49 | 27 | 25 | 9.4 | 0.0089 | Up | |
| FLT1 | | 55 | 23 | 13 | 24 | 49 | 27 | 25 | 9.3 | 0.0092 | Up | |
| MAP4 | 4134 | 315 | 70 | 13 | 24 | 49 | 27 | 25 | 9.3 | 0.0094 | Up | |
| NUMB | 8650 | 143 | 35 | 13 | 24 | 51 | 24 | 27 | 8.4 | 0.0149 | Up | |
| TG737 | 8100 | 207 | 89 | 13 | 25 | 49 | 28 | 25 | 8.2 | 0.0160 | Up | |
| CG005 | 10443 | 153 | 75 | 13 | 24 | 44 | 38 | 20 | 11.4 | 0.0033 | | down |
| SYNGR1 | 9145 | 55 | 26 | 12 | 22 | 49 | 25 | 27 | 9.9 | 0.0070 | Up | |
| ORAOV2 | 55107 | 88 | 94 | 12 | 23 | 42 | 41 | 19 | 10.2 | 0.0060 | | down |
| METTL3 | 56339 | 89 | 26 | 12 | 22 | 42 | 40 | 20 | 9.1 | 0.0107 | | down |
| IMAGE: 4215339 | 113177 | 24 | 32 | 12 | 23 | 42 | 41 | 19 | 8.9 | 0.0113 | | down |
| GHITM | 227069 | 612 | 192 | −13 | −23 | 25 | 27 | 48 | 9.6 | 0.0081 | down | |
| WDR1 | 9948 | 754 | 179 | −13 | −23 | 25 | 27 | 48 | 8.8 | 0.0121 | down | |
| PBP | 5037 | 509 | 173 | −13 | −23 | 27 | 24 | 50 | 8.2 | 0.0169 | down | |
| MGC5247 | 79029 | 132 | 57 | −13 | −23 | 18 | 42 | 41 | 11.0 | 0.0040 | | Up |
| FLJ22457 | 79961 | 159 | 49 | −13 | −23 | 19 | 41 | 41 | 8.9 | 0.0118 | | Up |
| USP3 | 9960 | 271 | 75 | −13 | −23 | 20 | 38 | 43 | 7.9 | 0.0191 | | Up |
| U2AF2 | 11338 | 111 | 47 | −14 | −25 | 24 | 29 | 48 | 11.3 | 0.0034 | down | |
| TNFSF10 | 8743 | 159 | 82 | −14 | −25 | 25 | 25 | 50 | 9.5 | 0.0084 | down | |
| DHX15 | 1665 | 1164 | 291 | −14 | −25 | 24 | 29 | 48 | 8.6 | 0.0132 | down | |
| UCP2 | 7351 | 379 | 261 | −14 | −25 | 25 | 25 | 50 | 8.5 | 0.0139 | down | |
| MFHAS1 | 9258 | 178 | 71 | −14 | −24 | 18 | 41 | 42 | 11.2 | 0.0036 | | Up |
| CCT2 | 10576 | 1156 | 310 | −14 | −25 | 20 | 36 | 45 | 11.0 | 0.0040 | | Up |
| TNFRSF10B | 8795 | 74 | 40 | −14 | −25 | 20 | 36 | 45 | 10.1 | 0.0065 | | Up |
| IL15RA | 3601 | 170 | 56 | −14 | −25 | 18 | 40 | 43 | 9.5 | 0.0086 | | Up |
| DHX15 | 1665 | 685 | 209 | −14 | −25 | 18 | 40 | 43 | 9.3 | 0.0097 | | Up |
| PSMD9 | 5715 | 292 | 83 | −14 | −25 | 20 | 36 | 45 | 8.6 | 0.0137 | | Up |
| ELP3 | 55140 | 78 | 24 | −14 | −25 | 20 | 36 | 45 | 8.2 | 0.0162 | | Up |
| TMEM4 | 10330 | 257 | 73 | −15 | −26 | 25 | 24 | 52 | 15.1 | 0.0005 | down | |
| ATP5B | 506 | 2062 | 631 | −15 | −26 | 25 | 24 | 52 | 14.4 | 0.0007 | down | |
| LAP3 | 41056 | 593 | 288 | −15 | −26 | 25 | 24 | 52 | 14.2 | 0.0008 | down | |
| BF | 629 | 246 | 142 | −15 | −26 | 22 | 31 | 48 | 12.3 | 0.0021 | down | Up |
| SORD | 6652 | 251 | 73 | −15 | −26 | 26 | 23 | 52 | 12.1 | 0.0023 | down | |
| WARS | 7453 | 303 | 214 | −15 | −26 | 25 | 24 | 52 | 11.7 | 0.0029 | down | |
| PIGR | 5284 | 473 | 567 | −15 | −26 | 22 | 31 | 48 | 11.3 | 0.0035 | down | Up |
| CLN6 | 91860 | 192 | 98 | −15 | −26 | 25 | 24 | 52 | 10.4 | 0.0054 | down | |
| AKR1A1 | 10327 | 535 | 179 | −15 | −26 | 22 | 31 | 48 | 9.9 | 0.0068 | down | Up |
| PBP | 5037 | 1006 | 597 | −15 | −26 | 22 | 31 | 48 | 9.7 | 0.0076 | down | Up |
| UQCRFS1 | 7386 | 1301 | 396 | −15 | −26 | 24 | 27 | 50 | 9.6 | 0.0080 | down | |
| MCM5 | 4174 | 95 | 221 | −15 | −26 | 22 | 31 | 48 | 9.4 | 0.0092 | down | Up |
| DKFZp566C0424 | 26099 | 86 | 46 | −15 | −26 | 24 | 27 | 50 | 9.1 | 0.0107 | down | |
| UQCRH | 7388 | 1537 | 506 | −15 | −26 | 22 | 31 | 48 | 8.9 | 0.0115 | down | Up |
| MMP3 | 4314 | 311 | 321 | −15 | −26 | 22 | 31 | 48 | 8.9 | 0.0117 | down | Up |
| ARL61P4 | | 192 | 45 | −15 | −26 | 22 | 31 | 48 | 8.5 | 0.0142 | down | Up |
| FLJ14827 | 84934 | 40 | 36 | −15 | −26 | 24 | 27 | 50 | 8.4 | 0.0153 | down | |
| FDFT1 | 2222 | 938 | 477 | −15 | −26 | 22 | 31 | 48 | 8.0 | 0.0181 | down | Up |
| H2AFZ | 3015 | 1025 | 472 | −15 | −26 | 24 | 27 | 50 | 7.9 | 0.0186 | down | |
| DKFZP564G2022 | 25963 | 576 | 318 | −15 | −26 | 18 | 38 | 45 | 13.0 | 0.0015 | | Up |
| RPS27L | 51065 | 1489 | 480 | −15 | −26 | 20 | 35 | 46 | 12.2 | 0.0022 | | Up |
| KIAA1277 | 27147 | 49 | 31 | −15 | −26 | 20 | 35 | 46 | 11.1 | 0.0039 | | Up |
| UBE2L6 | 9246 | 435 | 200 | −15 | −26 | 18 | 38 | 45 | 10.4 | 0.0055 | | Up |
| KIF13B | 23303 | 220 | 154 | −15 | −26 | 20 | 35 | 46 | 8.1 | 0.0167 | | Up |
| XPO7 | 23039 | 132 | 36 | −16 | −28 | 20 | 33 | 48 | 17.3 | 0.0002 | down | Up |
| Clorf16 | 9887 | 56 | 33 | −16 | −27 | 22 | 29 | 49 | 13.6 | 0.0011 | down | Up |
| TEGT | 7009 | 1413 | 336 | −16 | −28 | 25 | 22 | 54 | 11.8 | 0.0027 | down | Up |
| RQCD1 | | 48 | 32 | −16 | −28 | 20 | 33 | 48 | 11.4 | 0.0033 | down | Up |
| KIAA0826 | 285527 | 317 | 151 | −16 | −28 | 22 | 29 | 50 | 11.2 | 0.0036 | down | Up |
| LAMP3 | 27074 | 72 | 46 | −16 | −28 | 22 | 29 | 50 | 11.1 | 0.0038 | down | Up |
| SQRDL | 58472 | 643 | 317 | −16 | −28 | 18 | 36 | 46 | 11.2 | 0.0038 | down | Up |
| NAT1 | 9 | 165 | 81 | −16 | −28 | 20 | 33 | 48 | 10.7 | 0.0047 | down | Up |
| NSEP1 | 4904 | 1957 | 735 | −16 | −28 | 20 | 33 | 48 | 10.5 | 0.0052 | down | Up |
| KIAA0746 | | 439 | 137 | −16 | −28 | 20 | 33 | 48 | 9.5 | 0.0085 | down | Up |
| CCNA2 | 890 | 172 | 85 | −16 | −28 | 20 | 33 | 48 | 9.4 | 0.0087 | down | Up |
| ISG20 | 3669 | 174 | 102 | −16 | −28 | 20 | 33 | 48 | 9.2 | 0.0099 | down | Up |
| MTSG1 | 57509 | 283 | 121 | −16 | −28 | 18 | 36 | 46 | 9.0 | 0.0110 | down | Up |
| AP3D1 | 8943 | 56 | 21 | −16 | −26 | 22 | 30 | 48 | 9.0 | 0.0113 | down | Up |
| LOC203069 | 203069 | 108 | 35 | −16 | −28 | 22 | 29 | 50 | 8.9 | 0.0116 | down | Up |
| SRP72 | 6731 | 574 | 143 | −16 | −28 | 20 | 33 | 48 | 8.8 | 0.0125 | down | Up |
| — | 1534 | 370 | 103 | −16 | −28 | 18 | 36 | 46 | 8.4 | 0.0147 | down | Up |
| SSNA1 | 8636 | 183 | 47 | −16 | −28 | 20 | 33 | 48 | 8.0 | 0.0183 | down | Up |
| FLJ10525 | 55161 | 206 | 67 | −16 | −28 | 22 | 29 | 50 | 7.8 | 0.0197 | down | |
| TEGT | 7009 | 1252 | 328 | −16 | −28 | 15 | 44 | 43 | 23.1 | 0.0001 | | Up |
| FLJ10579 | 55177 | 228 | 52 | −16 | −28 | 16 | 40 | 45 | 13.6 | 0.0011 | | Up |
| FLJ20699 | 55020 | 114 | 72 | −16 | −28 | 16 | 40 | 45 | 9.2 | 0.0099 | | Up |
| MYRIP | 25924 | 30 | 26 | −16 | −27 | 22 | 30 | 49 | 8.7 | 0.0131 | | Up |
| DAZAP2 | 9802 | 1313 | 275 | −17 | −30 | 20 | 31 | 50 | 15.5 | 0.0004 | down | Up |
| UMP-CMPK | 51727 | 800 | 256 | −17 | −30 | 24 | 24 | 54 | 14.9 | 0.0006 | down | |
| MRPL46 | 26589 | 147 | 43 | −17 | −30 | 24 | 24 | 54 | 14.3 | 0.0008 | down | |
| FLJ20452 | 54939 | 133 | 53 | −17 | −30 | 16 | 38 | 46 | 13.7 | 0.0011 | down | Up |
| GCHFR | 2644 | 122 | 54 | −17 | −30 | 20 | 31 | 50 | 13.1 | 0.0015 | down | Up |
| ASNA1 | 439 | 214 | 51 | −17 | −30 | 20 | 31 | 50 | 11.7 | 0.0028 | down | Up |
| TAPBP-R | 55080 | 108 | 54 | −17 | −30 | 16 | 38 | 46 | 11.1 | 0.0039 | down | Up |
| TOPK | 55872 | 148 | 100 | −17 | −30 | 16 | 38 | 46 | 11.0 | 0.0041 | down | Up |
| — | 5265 | 990 | 1187 | −17 | −30 | 18 | 35 | 48 | 10.8 | 0.0046 | down | Up |
| SERPINA1 | 5265 | 619 | 873 | −17 | −30 | 18 | 35 | 48 | 10.7 | 0.0047 | down | Up |
| KIAA1185 | 57470 | 378 | 93 | −17 | −30 | 22 | 27 | 52 | 10.3 | 0.0057 | down | Up |
| SLC39A8 | 64116 | 175 | 111 | −17 | −30 | 18 | 35 | 48 | 9.2 | 0.0100 | down | Up |
| KIAA0746 | | 765 | 256 | −17 | −30 | 18 | 35 | 48 | 8.5 | 0.0141 | down | Up |
| MUF1 | 10489 | 156 | 48 | −17 | −30 | 20 | 31 | 50 | 7.8 | 0.0195 | down | Up |
| HAN11 | 10238 | 83 | 28 | −17 | −30 | 15 | 42 | 45 | 14.2 | 0.0008 | | Up |
| CNOT7 | 29883 | 509 | 189 | −17 | −30 | 15 | 42 | 45 | 10.1 | 0.0065 | | Up |
| PIGT | 51604 | 440 | 160 | −17 | −30 | 15 | 42 | 45 | 9.5 | 0.0084 | | Up |
| GSR | 2936 | 132 | 92 | −18 | −32 | 18 | 33 | 50 | 15.5 | 0.0004 | down | Up |
| ITGAE | 3682 | 315 | 111 | −18 | −32 | 15 | 40 | 46 | 13.4 | 0.0012 | down | Up |
| CASP1 | 834 | 92 | 74 | −18 | −32 | 18 | 33 | 50 | 12.1 | 0.0024 | down | Up |
| UCP2 | 7351 | 271 | 102 | −18 | −32 | 18 | 33 | 50 | 11.5 | 0.0032 | down | Up |
| DDX23 | 9416 | 171 | 76 | −18 | −32 | 18 | 33 | 50 | 11.2 | 0.0037 | down | Up |
| CGI-51 | | 146 | 208 | −18 | −32 | 18 | 33 | 50 | 9.8 | 0.0074 | down | Up |
| COX5A | 9377 | 1135 | 354 | −19 | −34 | 20 | 27 | 54 | 16.3 | 0.0003 | down | Up |
| MAPKAPK5 | 8550 | 162 | 50 | −19 | −34 | 16 | 35 | 50 | 15.9 | 0.0004 | down | Up |
| MCSR1 | 10445 | 202 | 63 | −19 | −34 | 20 | 27 | 54 | 13.9 | 0.0010 | down | Up |
| — | | 2673 | 736 | −19 | −34 | 20 | 27 | 54 | 13.3 | 0.0012 | down | Up |
| SLC25A3 | 5250 | 2389 | 573 | −19 | −34 | 16 | 35 | 50 | 13.4 | 0.0012 | down | Up |
| PSMA5 | 5686 | 591 | 189 | −19 | −34 | 16 | 35 | 50 | 13.2 | 0.0013 | down | Up |
| CXCL11 | 6373 | 116 | 210 | −19 | −34 | 20 | 27 | 54 | 12.5 | 0.0019 | down | Up |
| GRHPR | 9380 | 213 | 66 | −19 | −34 | 16 | 35 | 50 | 8.1 | 0.0169 | down | Up |
| SFPQ | 6421 | 716 | 188 | −20 | −34 | 22 | 22 | 56 | 16.6 | 0.0002 | down | Up |
| NDUFC1 | 4717 | 744 | 298 | −20 | −35 | 18 | 29 | 54 | 14.4 | 0.0007 | down | Up |
| C12orf8 | 10961 | 821 | 247 | −21 | −37 | 16 | 31 | 54 | 17.7 | 0.0001 | down | Up |
| SLC39A8 | 64116 | 233 | 154 | −21 | −37 | 18 | 27 | 55 | 15.7 | 0.0004 | down | Up |
| MGC5576 | 79022 | 577 | 215 | −23 | −41 | 20 | 20 | 61 | 30.6 | 0.0001 | down | Up |

Using the above-described methods, genes having expression levels above the average tumor expression level and genes having expression levels below the average tumor expression level in samples derived from patients who generally had poor outcome were discovered. The final list of validated genes was sorted by chromosomal location to identify consistent patterns of over or under expression that were chromosome location specific.

FIG. 4 is a scatterplot graphing the predicted survival outcome for the 166 stage I-IV primary colon cancers based on the 71 gene predictor set determined as outlined above. The x-axis of the plot depicts the percentage of genes for a given tumor sample that had expression values associated with a bad disease outcome. The y-axis of the plot depicted the percentage of genes for a given tumor sample that had expression values associated with a good disease outcome. Tumor samples from DOD patients (n=56) are represented by squares and all other samples (i.e., non-DOD) are represented by diamonds. Group 1 had good prognosis with only 6% being categorized as DOD. Group 4 had poor prognosis with 70% being categorized as DOD. Groups 2 and 3 had intermediate prognosis levels. Treatment, therefore, could be tailored to expected survival outcome as illustrated in the figure.

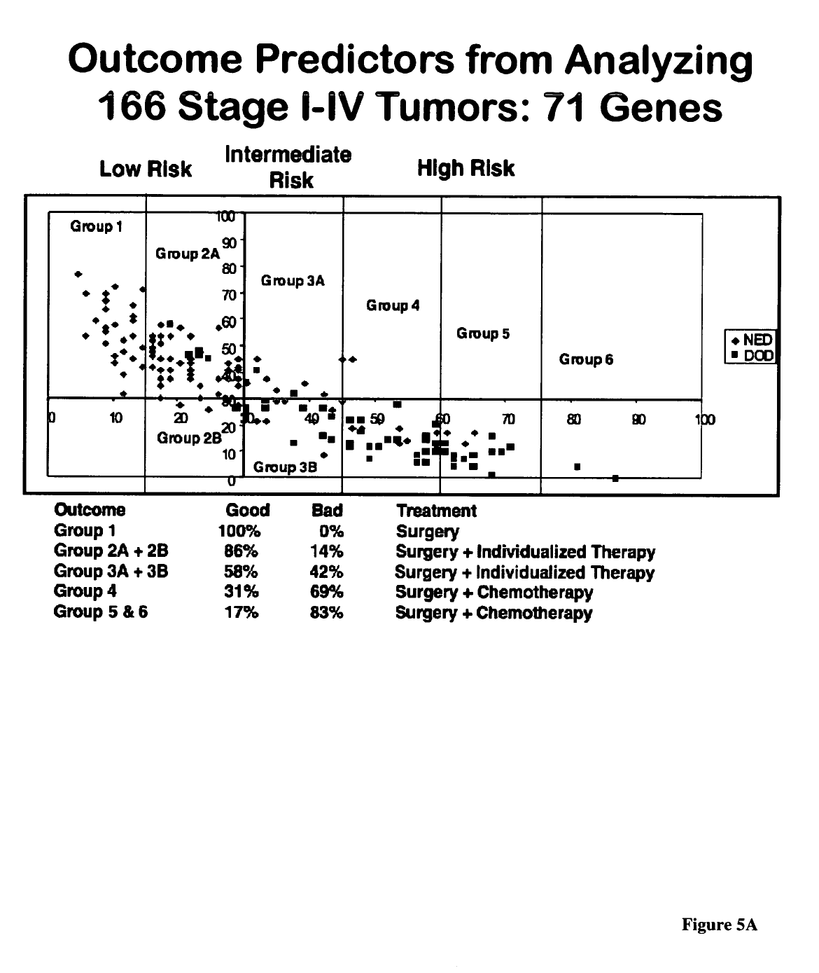

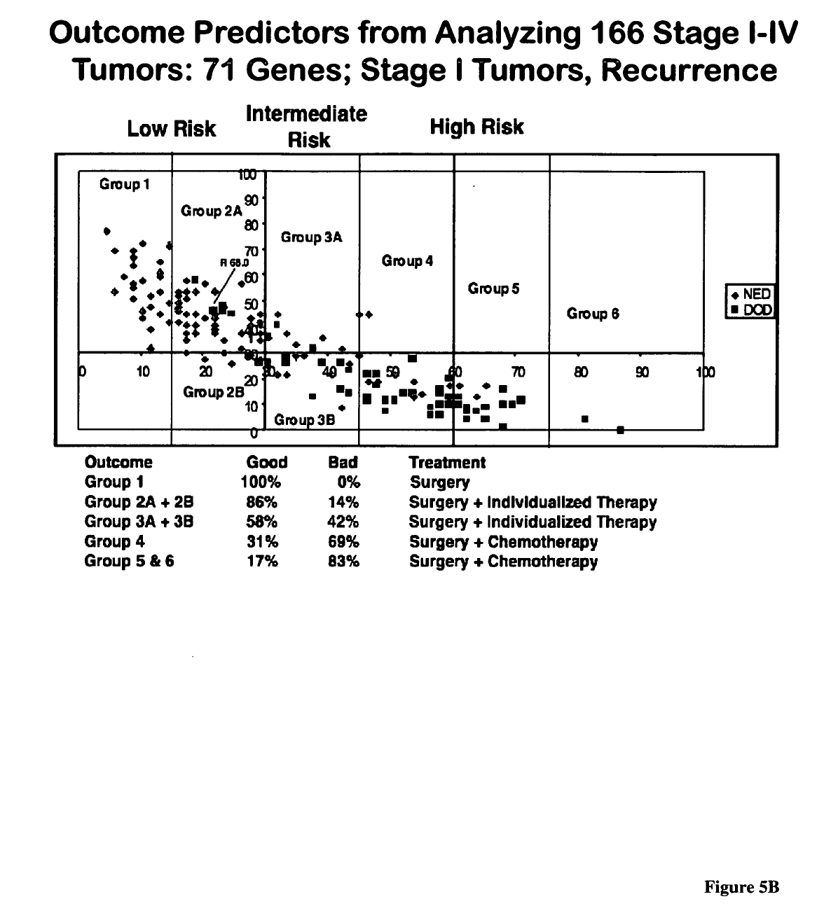

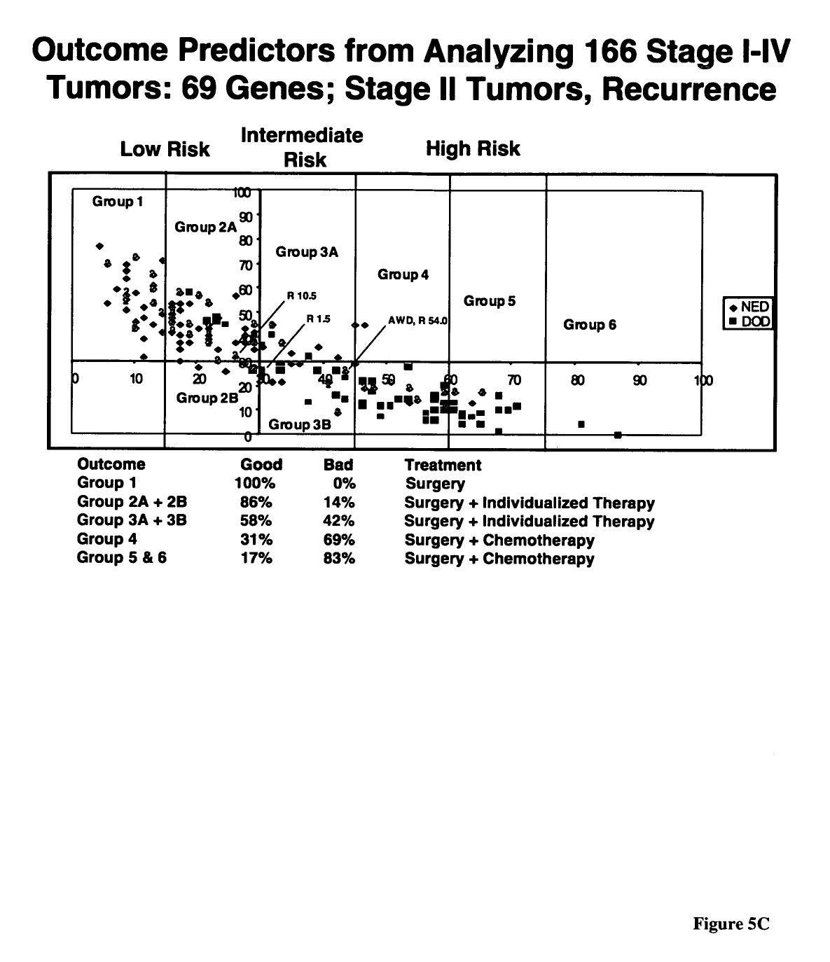

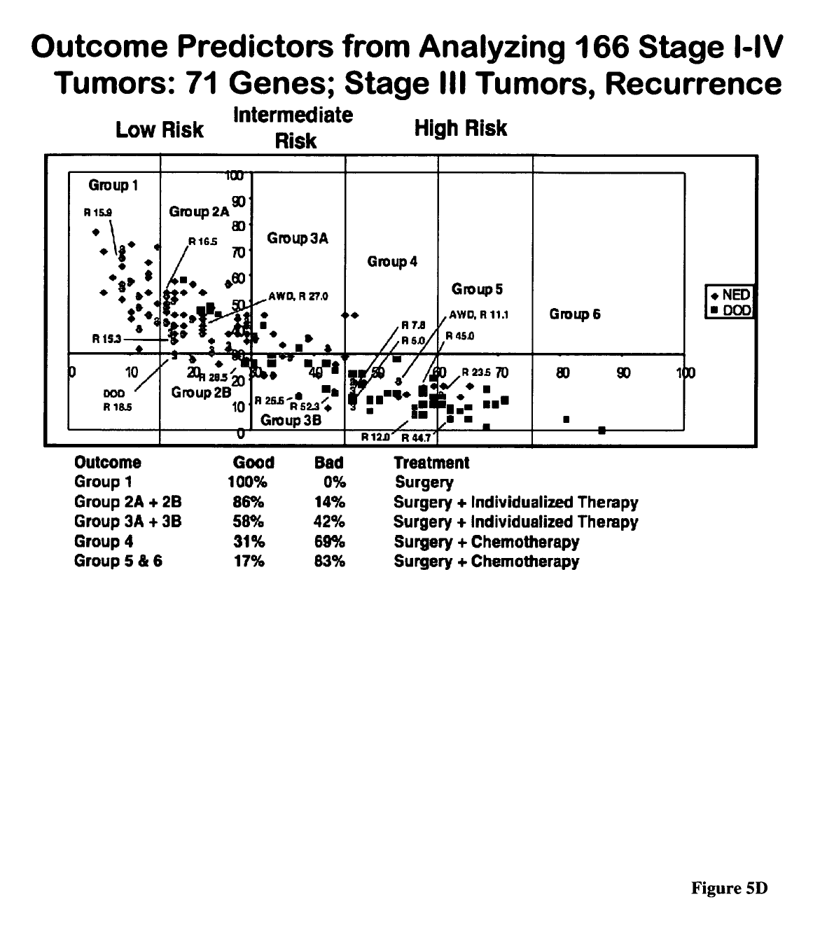

FIGS. 5A-E are scatterplots graphing the predicted outcomes for the 166 stage I-IV primary colon cancer tumor samples based on gene expression levels of the 71-genes in the 71-gene predictor set. The percentage of DOD patients increases steadily in each subgroup from Group 1 (0%) to Group 2A+2B (14%) to Group 3A+3B (42%) to Group 4 (69%) to Group 5+6 (83%). In FIGS. 5B, 5C, 5D and 5E, stage I, II, III and IV tumors are identified, respectively, and demonstrate binning is somewhat based on stage.

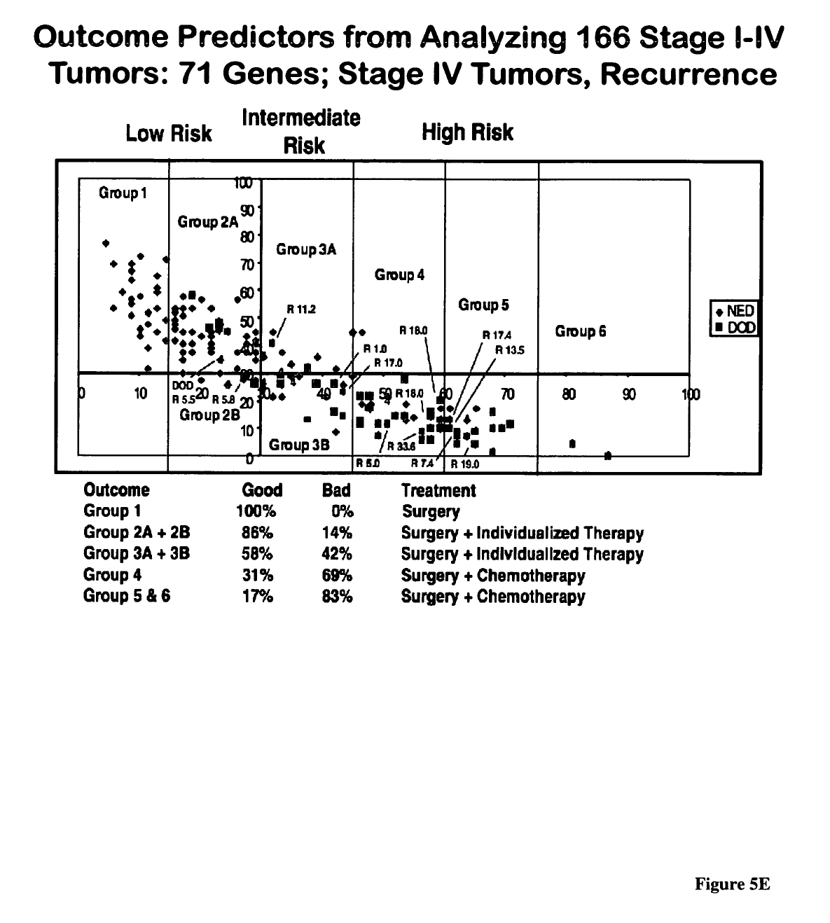

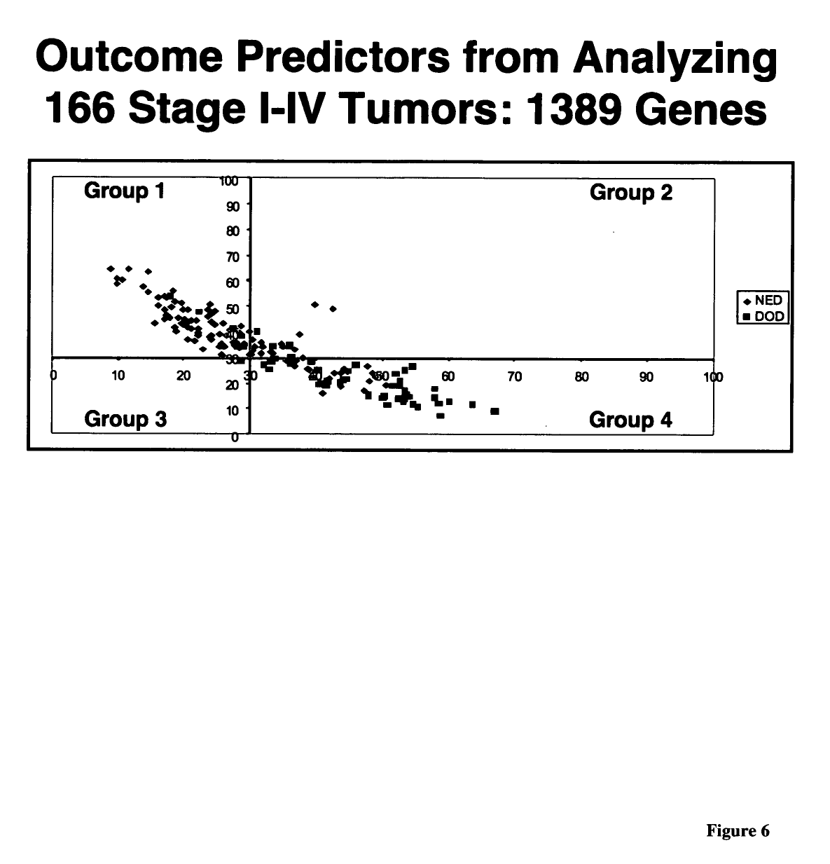

FIG. 6 is a scatterplot graphing the predicted outcome for the 166 stage I-IV primary colon cancer tumor samples based on gene expression levels of the 1389-genes in the 1389-gene predictor set. The stratification of survival outcome did not improve significantly between the 71 gene set and the 1389 gene set.

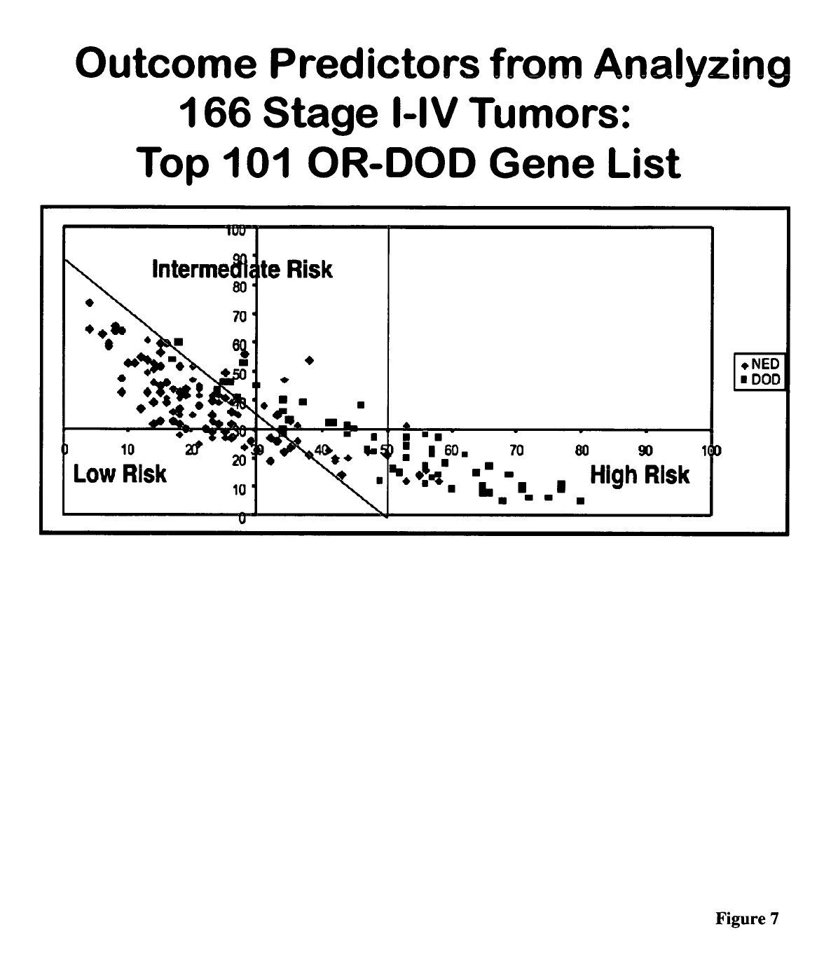

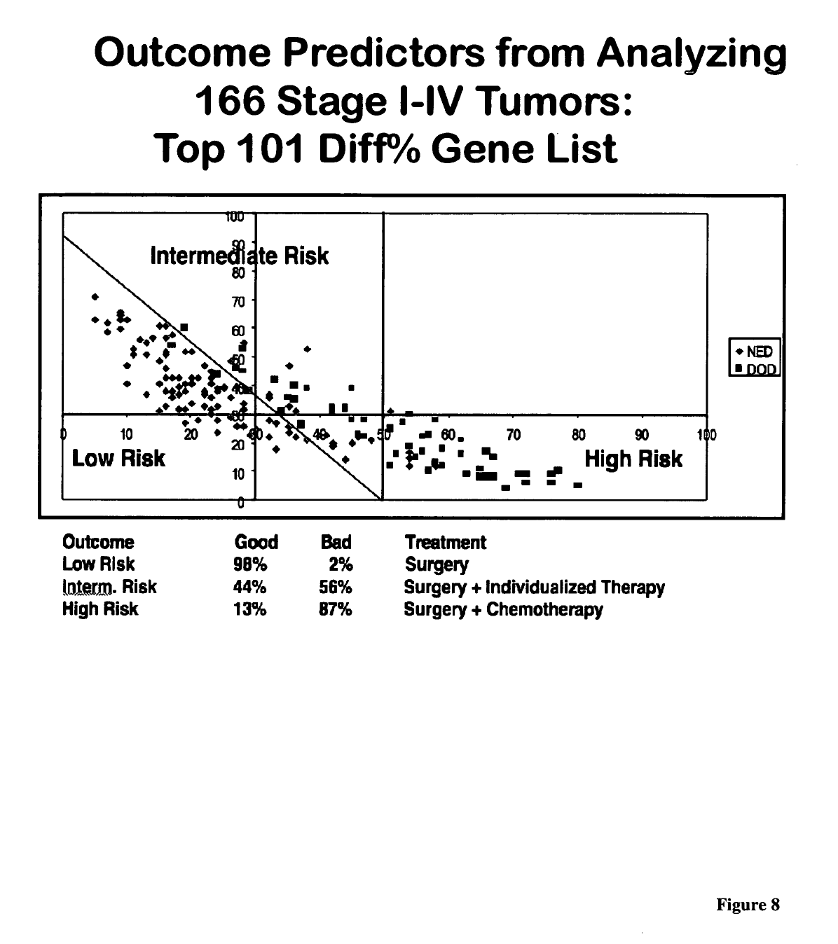

FIGS. 7 and 8 are scatterplots graphing the predicted outcome for the 166 stage I-IV primary colon cancer tumor samples based on gene expression levels of the 101 genes in the 101-gene predictor set ranked by the odds ratio analysis. The low risk category can be segregated from the intermediate and high risk categories by the lines indicated on the graph. The low risk category had 2% of patients who were in the DOD category. The high risk group by contrast had 87% of patients in the DOD category. The intermediate risk had 56% of patients in the DOD category. The predicted outcome for each patient can be used to tailor an individualized treatment plan for the patient as shown below each scatterplot.

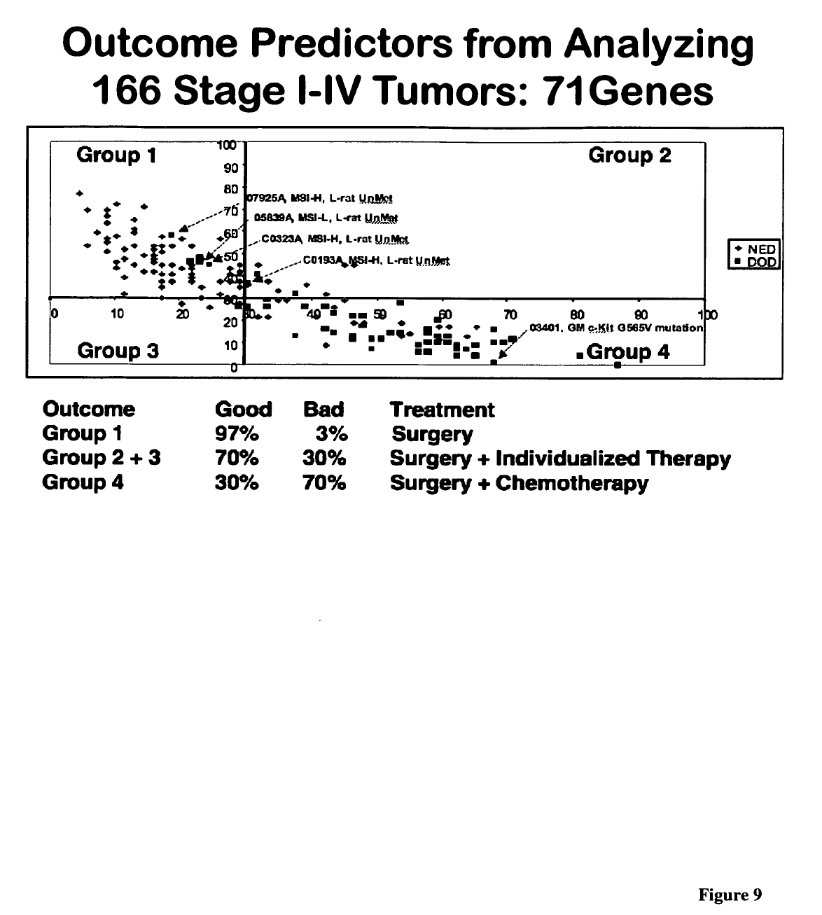

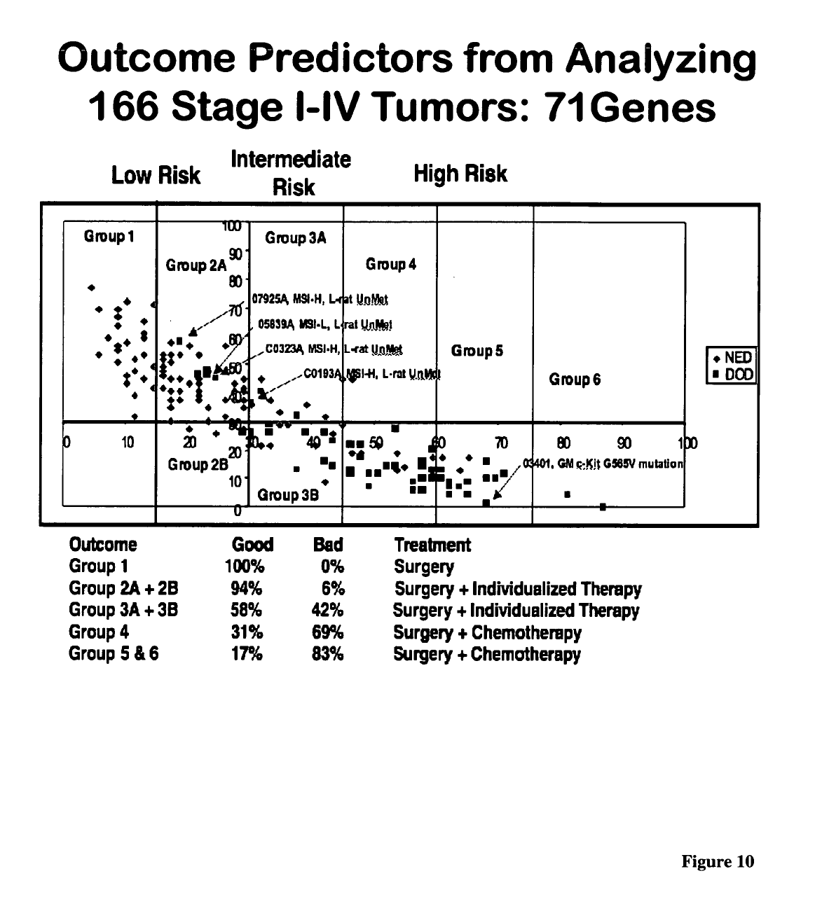

FIGS. 9 and 10 are scatterplots graphing the predicted outcome for the 166 stage I-IV primary colon cancer tumor samples based on gene expression levels of the 71 genes in the 71-gene predictor set as shown in FIG. 4 with LRAT methylation status of various samples identified. Several DOD samples that had binned to group 1 based on gene expression levels had low to no LRAT methylation, which predicts poor prognosis. Removing these samples from group 1 based on LRAT methylation status improved the performance of the prognosis prediction in the low risk category. The low risk category in this analysis only had 3% of patients in the DOD category. The low risk groups had excellent prediction of good outcome. Group 1 does not contain patients with DOD status while Group 2A+2B only has 6% of patients with DOD status.

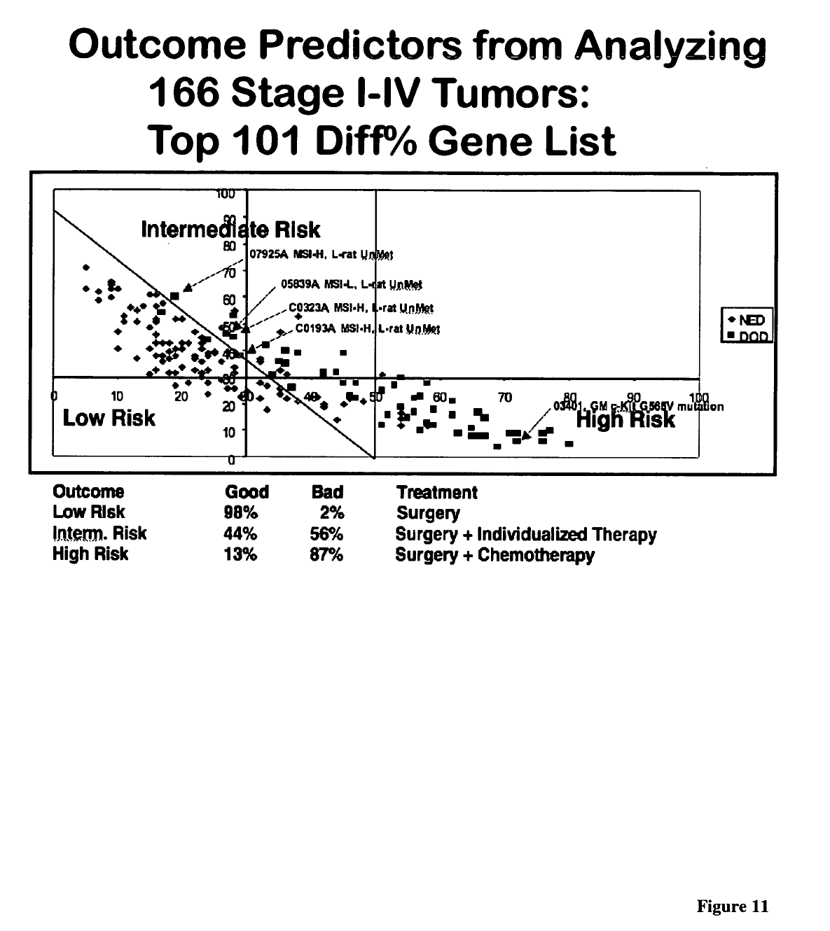

FIG. 11 is a scatterplot graphing the predicted outcome for the 166 stage I-IV primary colon cancer tumor samples based on gene expression levels of the 101 genes in the 101-gene predictor set ranked by difference score. Inclusion of LRAT methylation status was useful to reclassify some patient outcomes and improve the fidelity of prediction.

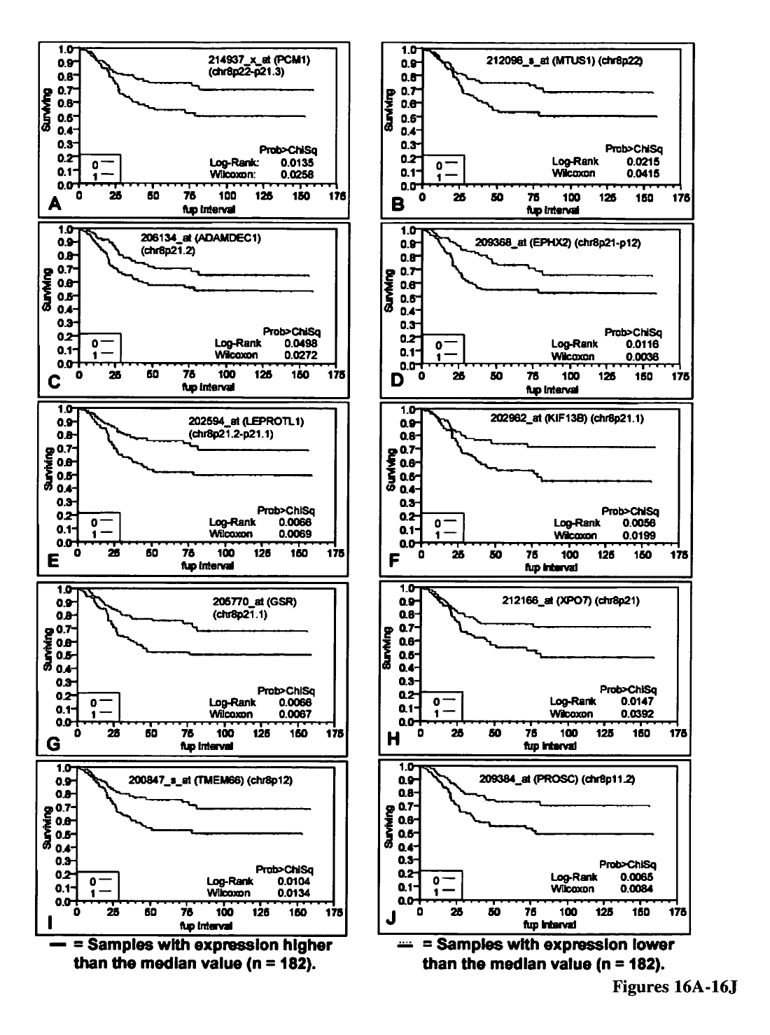

FIG. 16 shows the Kaplan Meier curves of genes found on the highly dysregulated chromosomal arm 8p. These genes, predictive of patient outcome, were identified from SNP and aberration studies from 89 tumor samples. In each case loss of expression of these genes was predictive of worse outcome, consistent with the common loss of the 8p chromosomal arm, where these genes are located.

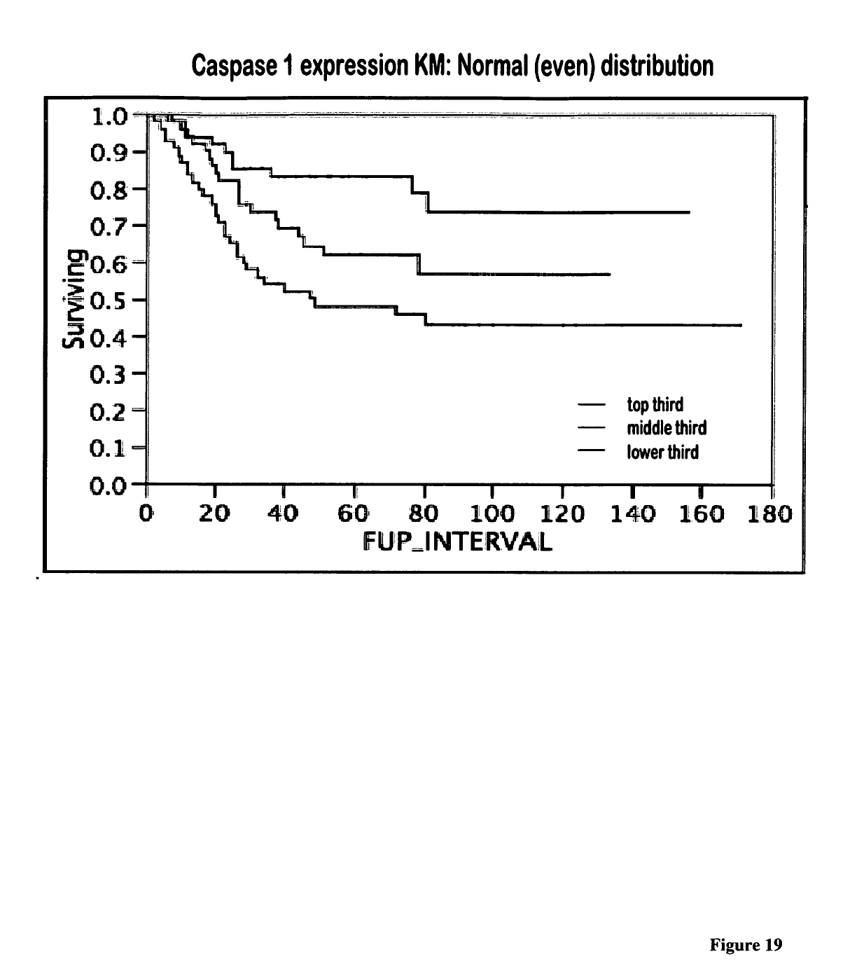

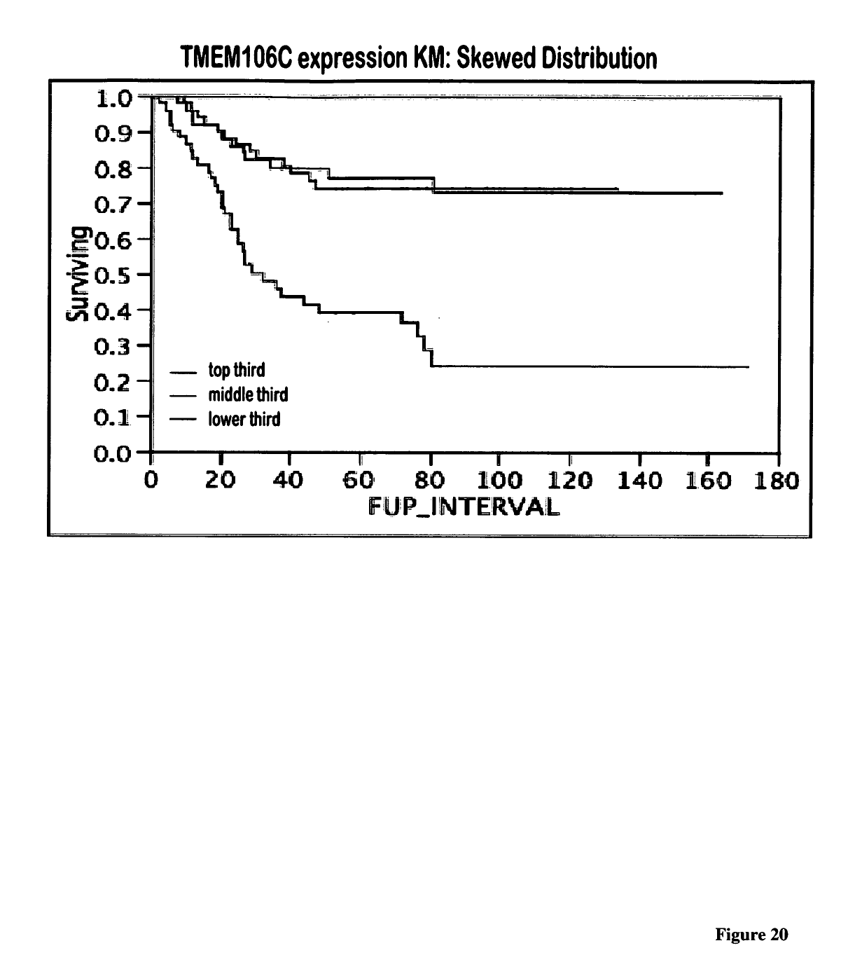

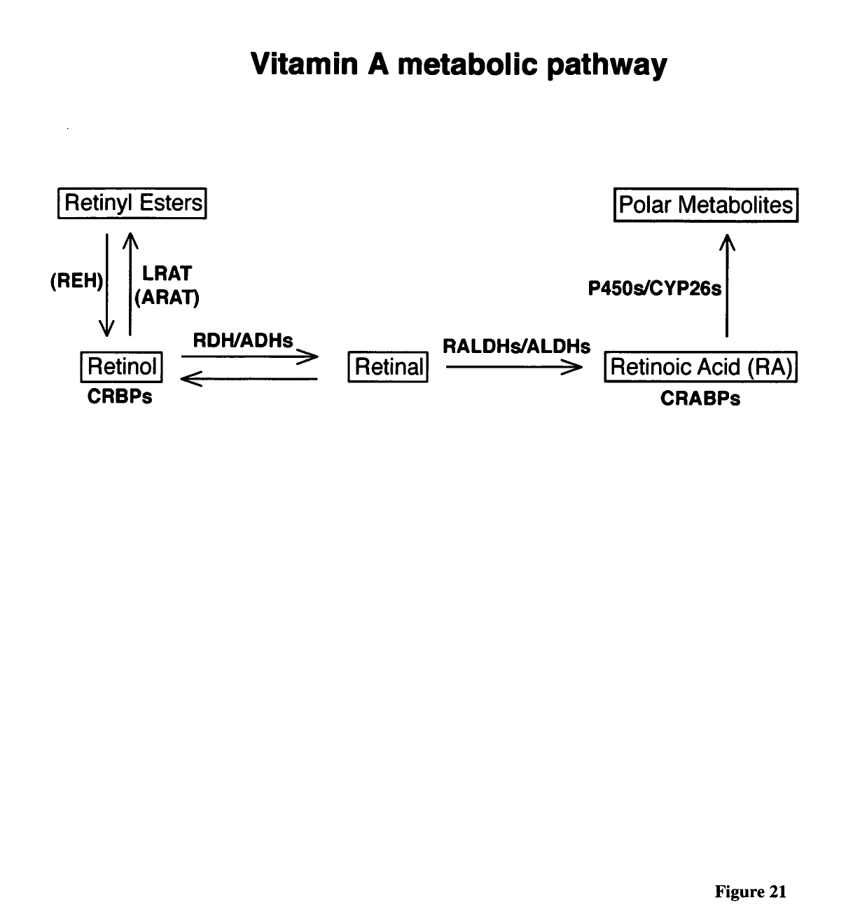

Typically, Kaplan Meier curves revealed expression patterns with normal distribution (FIG. 19) or skewed distribution (FIG. 20), when expression levels were split into top, middle and bottom thirds.

### Example 7

### Validation of Genes That Predict Disease Outcome in Patients Having Colon Cancer

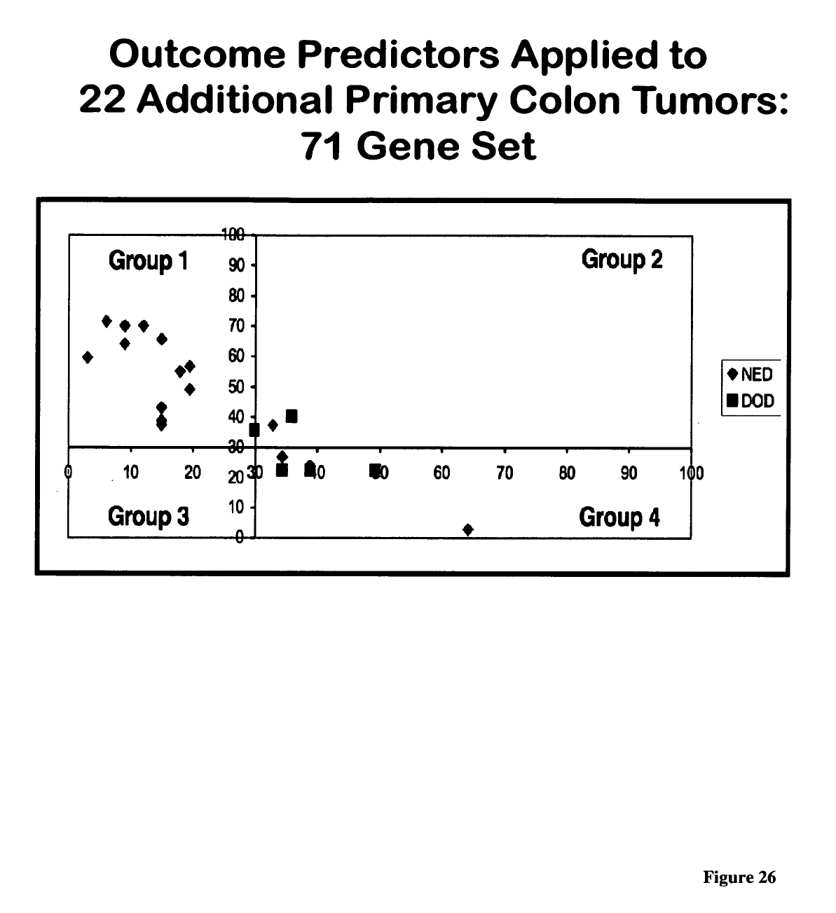

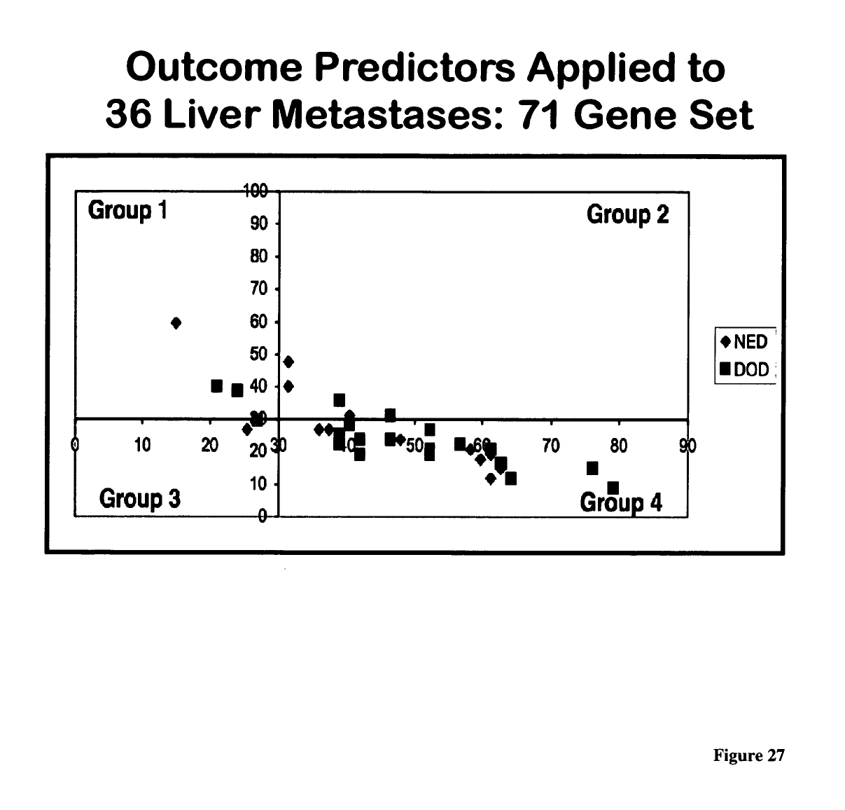

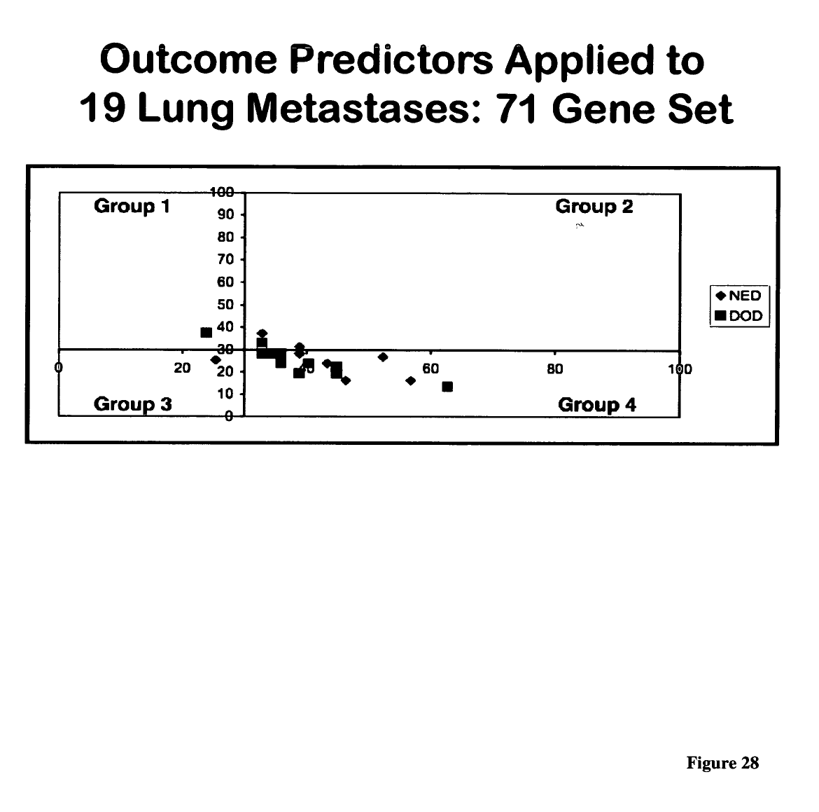

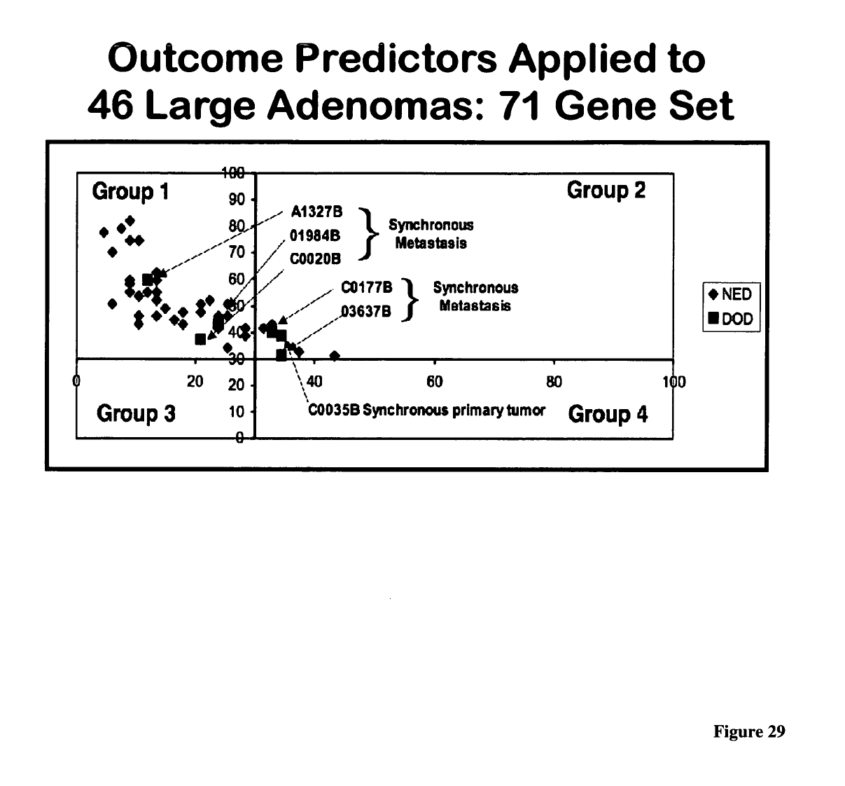

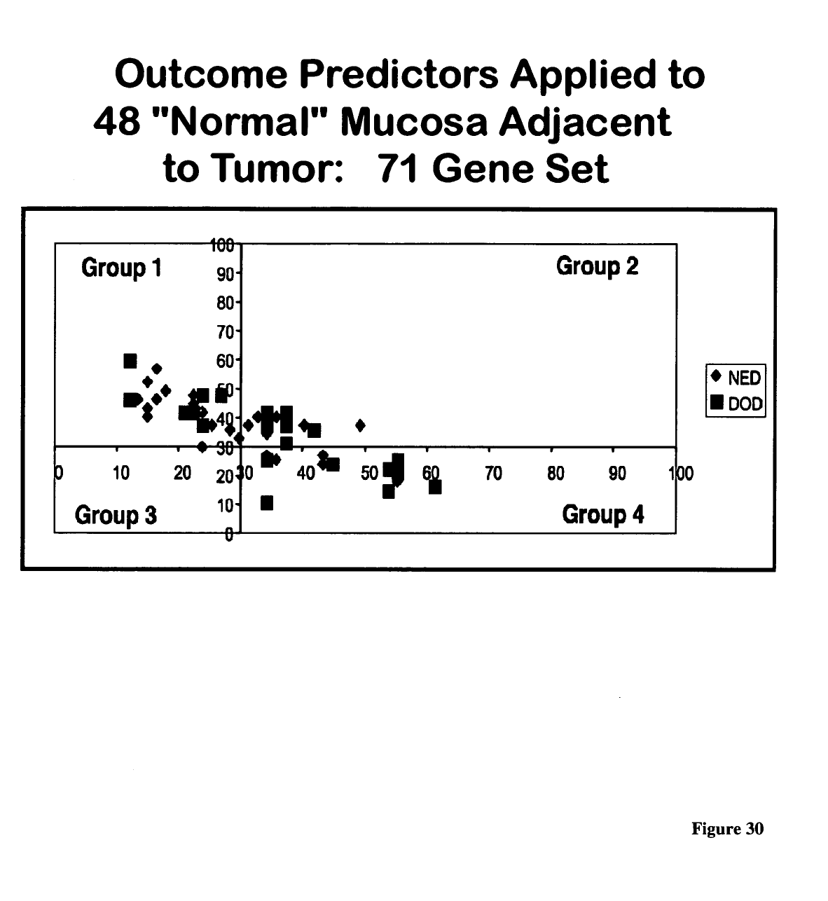

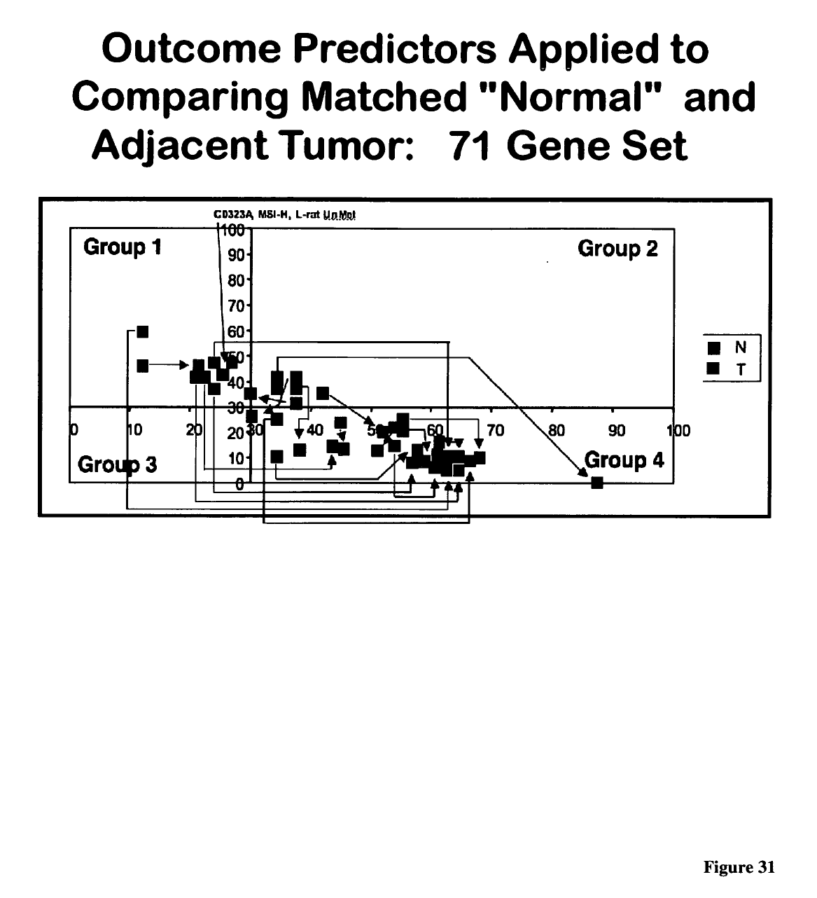

An additional 22 samples (FIG. 26), that were not included in the initial analysis, were used to validate the 71 gene list predictor set. None of the patient samples that binned to Group 1, where the prediction is for a good outcome, were derived from patients who DOD. Liver (FIG. 27) and lung (FIG. 28) metastases samples, largely binned to Group 4 when assessed for gene expression using the 71 gene predictor set. Large adenomas (FIG. 29), binned to group 1 in the majority of cases, unless there was the presence of synchronous metastases or tumor, consistent with early disease. Matched normal mucosa tissue (FIG. 30), adjacent to tumor, but no less than 10 cm from the tumor, when applied to the outcome predictor 71 gene set, binned to the various groups dependent upon outcome, possibly predicting a field effect or patient predisposition using expression profiling. FIG. 31 shows matched normal and tumor samples from the same patient, and the “direction” the expression profile of the outcome predictor 71 gene list, travels from normal to tumor samples, as indicated by the arrows. Typically, the normal tissue predicts a “better” outcome than the tumor tissue, again validating a role for this list of genes in tumor progression.

### Example 8

### Human Mapping (SNP) Array

SNP analysis was performed using the Affymetrix GeneChip Human Mapping 50K array Xba 240 array (or SNP array) following the protocol provided by Affymetrix (“GeneChip Mapping 100K Assay Manual”). Briefly, 0.25 μg of genomic DNA was digested with XbaI. The digests were ligated, PCR-amplified (such that the products were in the range of 250 to 2,000 bp), fragmented, biotin-labeled, and hybridized to the array. As in the expression array protocol, the SNP arrays also underwent staining and washing in Fluidics Station 450 (FS450) with the use of SAPE (streptavidin-phycoerythrin) and biotinylated anti-streptavidin antibody. The arrays were scanned in GeneChip Scanner 3000 to generate the image (DAT) and cell intensity (CEL) files. The CEL files were imported to GeneChip Genotyping Analysis Software (GTYPE) ver 4.1 software to generate the SNP calls.

### Example 9

### DNA Copy Number Analysis

The functionalities of Chromosomal Copy Number Analysis Tool (GNAT) software are embedded in GTYPE and the concepts and algorithms are initially described by Huang et al., “Whole Genome DNA Copy Number Changes Identified by High Density Oligonucleotide Arrays,” Hum. Genomics 1(4):287-99 (2004), which is hereby incorporated by reference in its entirety. GNAT uses the probe intensity data, as well as the GDAS-produced SNP calls to generate both the Single Point Analysis (SPA) and Genomic Smoothed Analysis (GSA) copy number (CN) estimates and the corresponding p-values. In addition, GNAT also generates the measures of loss of heterozygosity (LOH) based on the SNP calls. Once the SNP genotype calls and copy number estimates were obtained using GTYPE and GNAT, the data was further processed to refine the copy number data and to provide LOH calls that accommodate tissue and/or DNA aberration heterogeneity resulting in partially changed DNA (e.g. DNA with single gains at a given location in some of the strands and copy-neutral in other strands of the same chromosomal location). Regions of variation in copy number data are identified by applying segmentation and spatial filtering algorithms. The results are not constrained to integers. Sample-specific copy neutral, gain, and loss levels are obtained. For the LOH analysis, the SNPs that undergo an actual loss of heterozygosity from a normal control sample to the case sample are taken as input together with the SNPs that remain heterozygous. The majority of SNPs which are homozygous in the normal sample are ignored, as they are uninformative for regions of LOH. These two kind of SNPs are spatially averaged to allow for the effects of tissue heterogeneity. For those samples that lack a matched normal sample, the LOH values are inferred from the homozygosity data based on the relationship between these two quantities obtained from the matched tumor and normal samples.

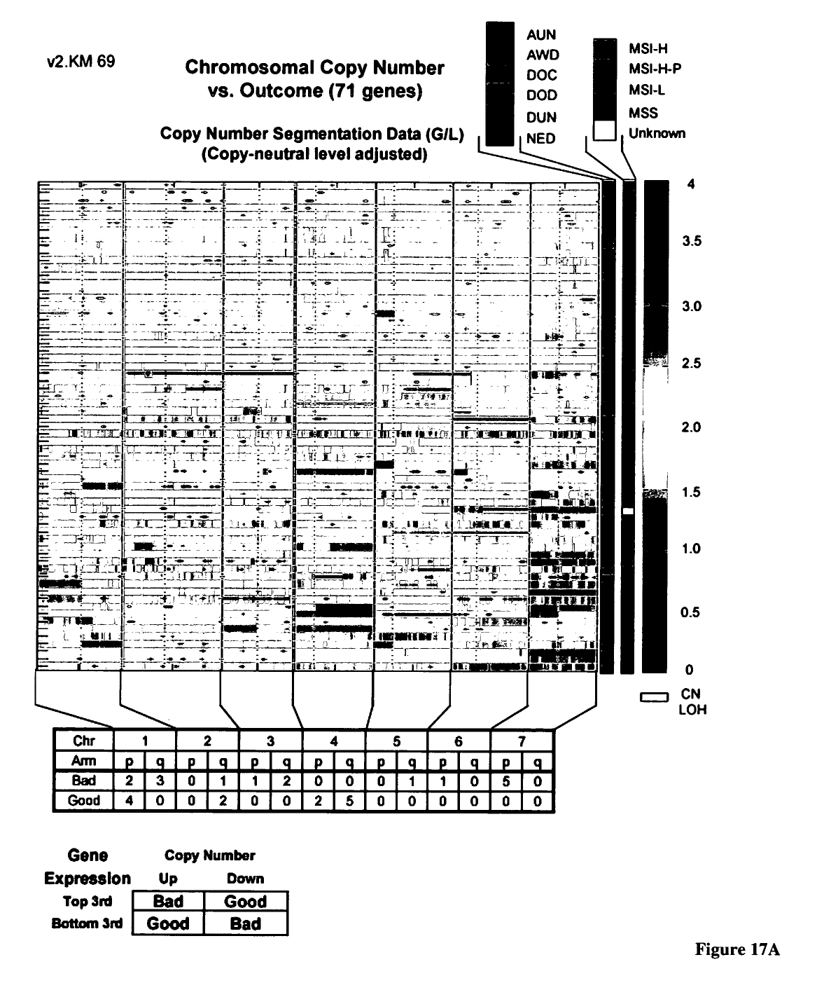

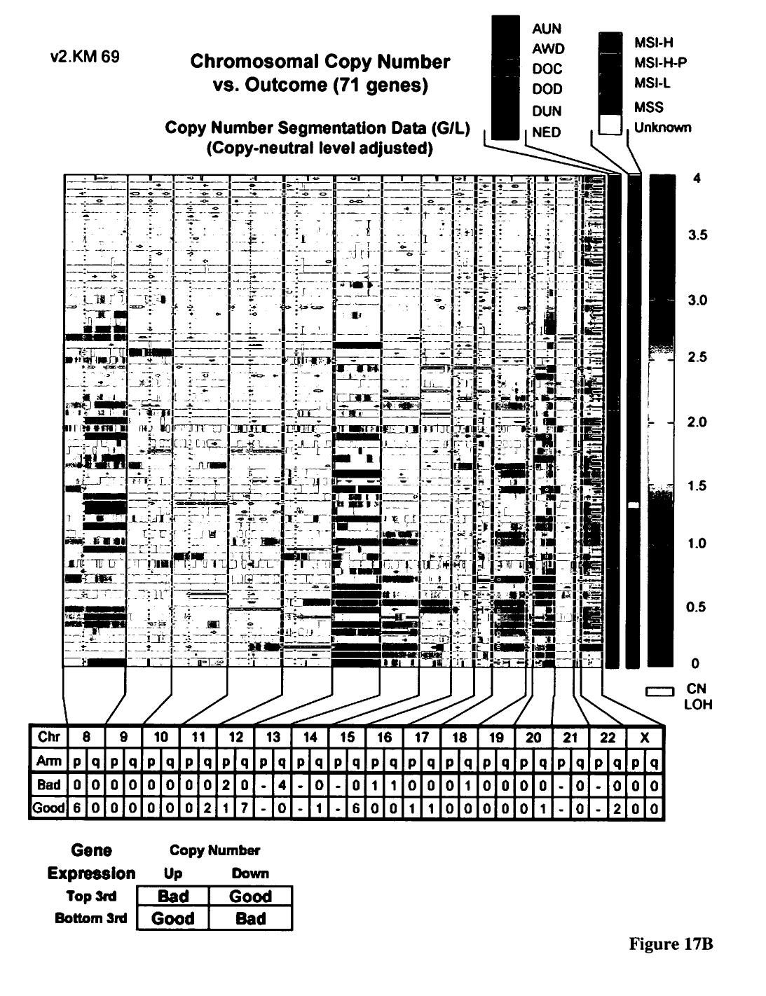

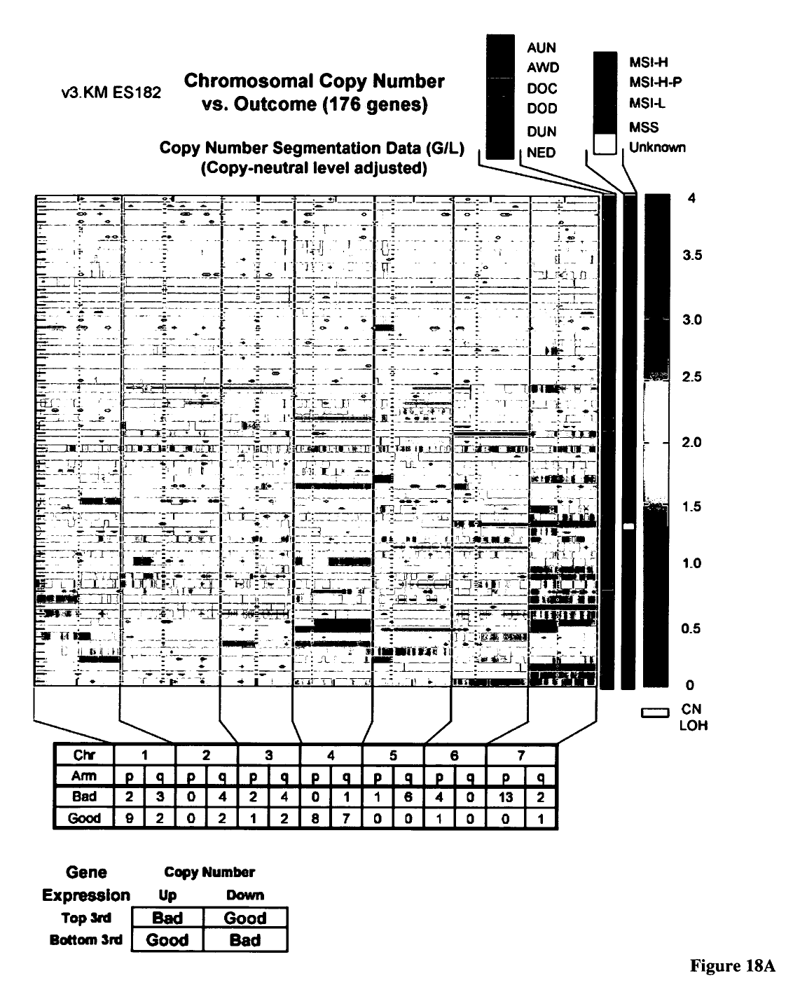

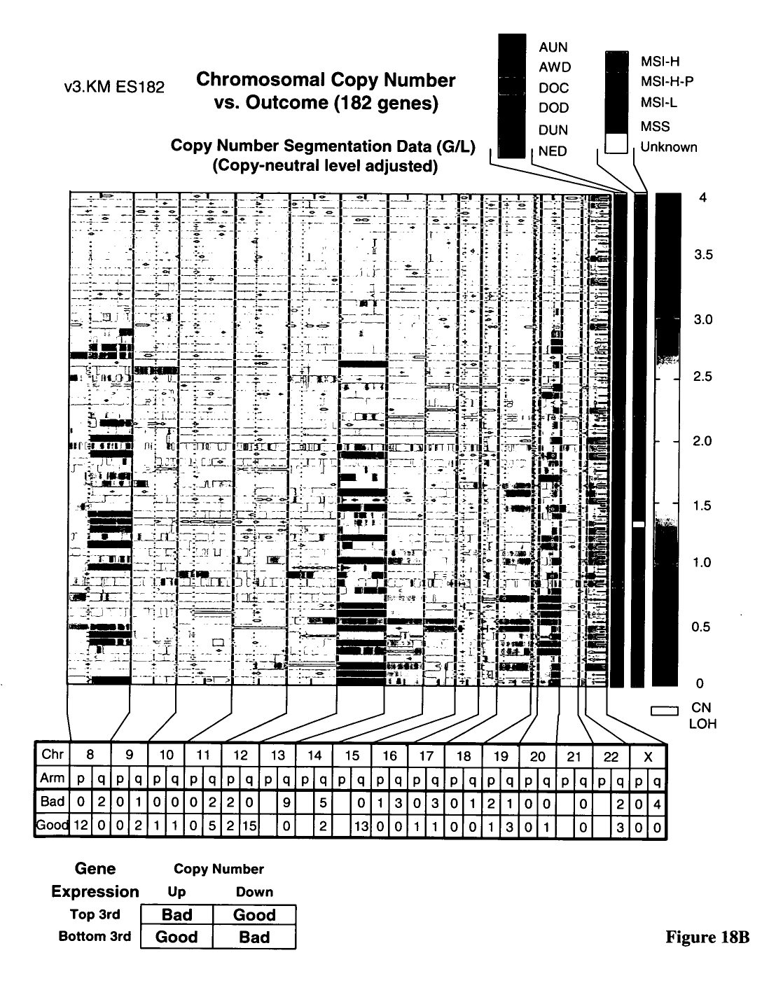

Shown in FIGS. 17 and 18 are heat maps depicting the chromosomal aberrations (gain, loss, copy neutral LOH) for each colorectal cancer sample analyzed by SNP arrays. Also indicated are each patient's clinical status (AUN, alive unknown; AWD, alive with disease; DOC, dead of other causes; DOD, dead of disease; DUN, dead of unknown disease; NED, no evidence of disease). Each figure also indicates the status of microsatellite instability for each sample, which can be classified as MSS (microsatellite stable), MSI-H (high level of microsatellite instability), MSI-L (low level of microsateliite instability), according to the 5 marker-criteria set by Bolan et al., “A National Cancer Institute Workshop on Microsatellite Instability for Cancer Detection and Familial Predisposition: Development of International Criteria for the Determination of Microsatellite Instability in Colorectal Cancer” Cancer Research 58:5248-57 (1998), which is hereby incorporated by reference in its entirety. In addition, a sample may be categorized as MSI-H-P (high level of microsatellite instability), in accordance to the three marker-criteria suggested by Nash et al., “Automated, Multiplex Assay for High-Frequency Microsatellite Instability in Colorectal Cancer” J Clin Oncol 21:3105-12 (2003), which is hereby incorporated by reference in its entirety.

### Example 10

### Gene Expression Dysregulation in Regions of Chromosomal Aberrations

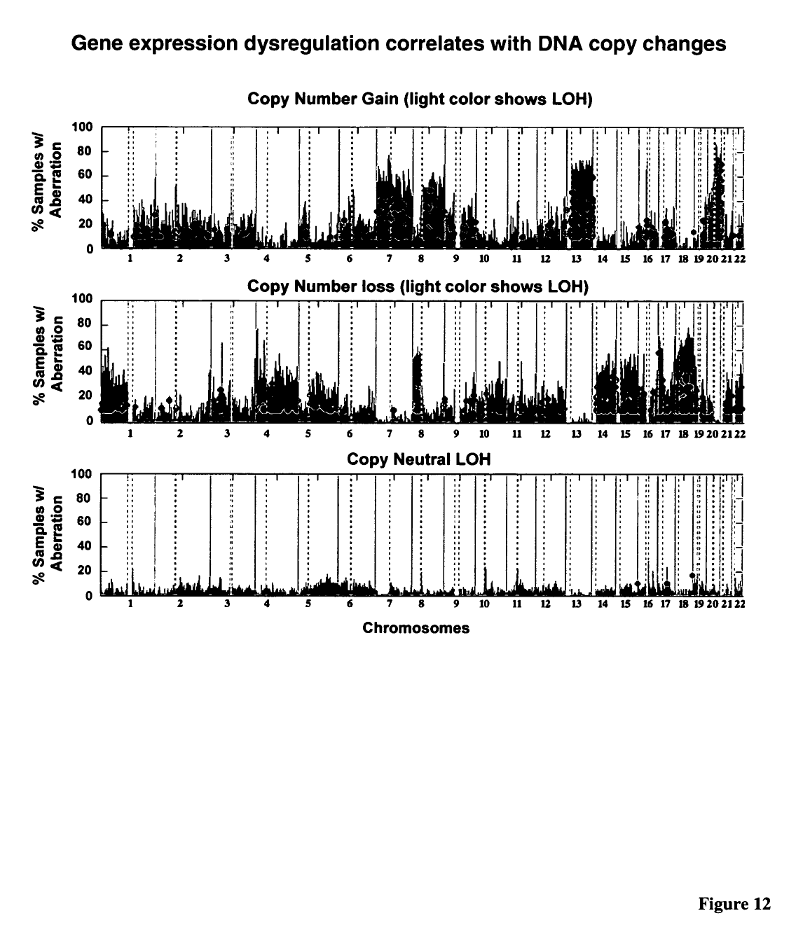

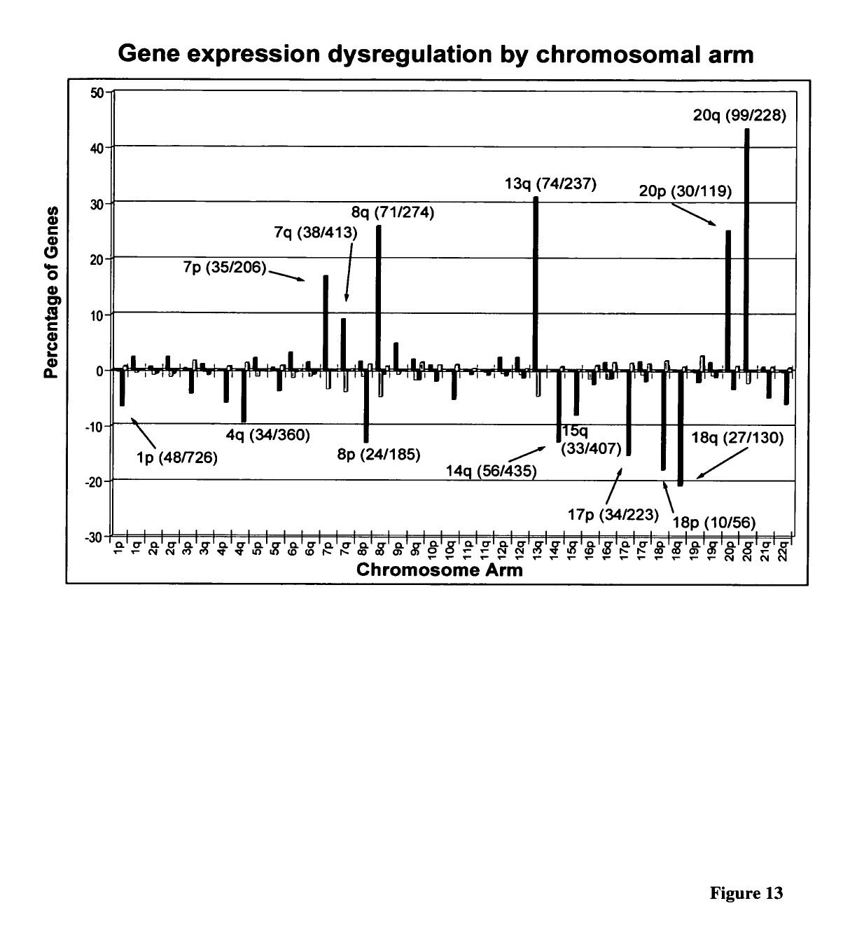

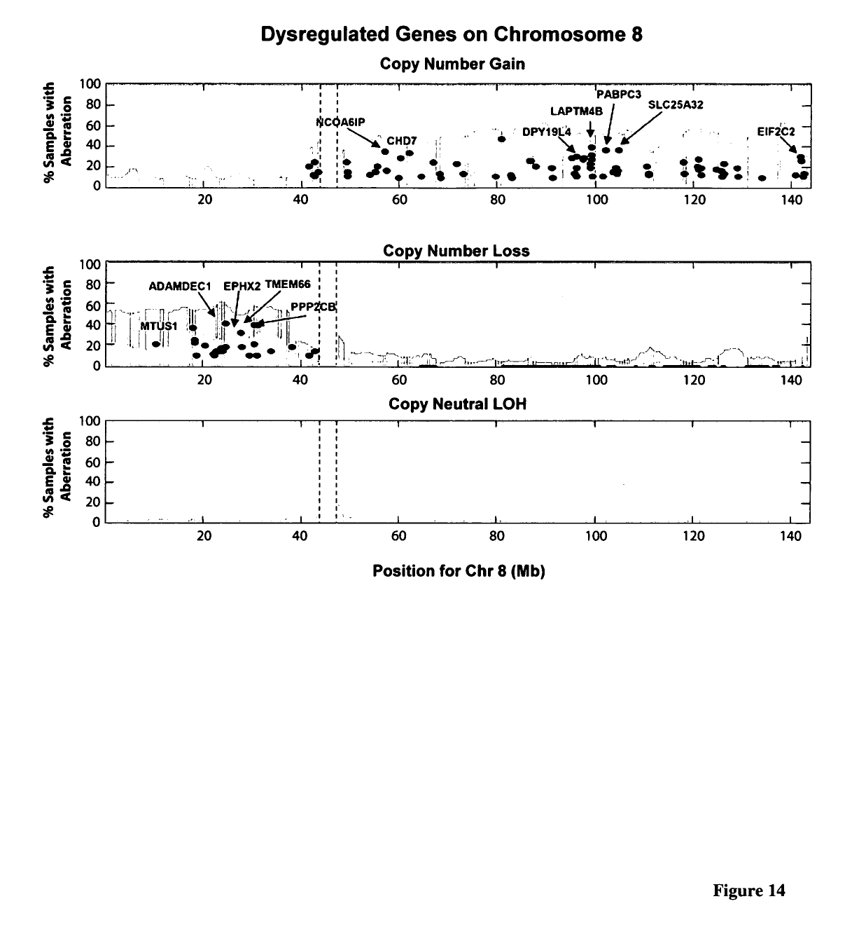

The simultaneous use of SNP and expression arrays allows one to analyze the patterns of gene expression in chromosomal regions usually characterized by aberrations (copy gains/losses involving either whole chromosomal arms, or regions of smaller size). Chromosomal arms 7p, 7q, 8q, 13q, 20p, and 20q, which usually gain additional copies in colorectal cancer, also have a high percentage of upregulated genes (see FIG. 13). On the other hand, the chromosomal arms 4q, 8p, 14q, 17p, 18p, and 18q, which are often lost in colorectal cancer, are marked by a high proportion of downregulated genes (FIG. 13). To determine if a gene is downregulated/upregulated, (zₚₛ)_{Ti}=(PS_{Ti}−AvePS_{N})/(σPS)_{N} was calculated, where PS_{Ti}; is the normalized intensity level of a probeset (ps) (which represents a given gene) for the tumor sample Ti, AvePS_{N} is the average intensity of a probeset (ps) among the normal mucosa samples, (σPS)_{N} is the standard deviation of the intensity of ps among the normal mucosa samples. The percent upregulation of a given gene (100 (# tumor samples with zₚₛ≧3)/71) and the percent downregulation of a given gene (100 (# tumor samples with zₚₛ≦3)/71) was also calculated. “71” refers to the number of tumor samples represented in both SNP and expression array analyses. In FIG. 12, a red circle represents a gene whose percent upregulation is at least 10, while a green circle represents a gene whose percent downregulation is at least 10. As shown in FIG. 13, the highest upregulation rates occur in the 20q, 13q, 8q, 20p, 7p, and 7q chromosome arms, while downregulation of genes is most often seen in 18p, 18q, 17p, 14q, 15q, 4q and 8p chromosome arms. Therefore, the direction of changes in gene expression levels is often consistent with the types of aberrations occurring in the chromosomal arms where these genes are located. The effect of copy number to gene expression is also illustrated in FIG. 14. As demonstrated in this figure, the often lost 8p arm is populated by genes with reduced levels of expression, while the usually gained 8q arm contains a high percentage of upregulated genes. The expression levels of the top disregulated genes in those aneuploid chromosomes/chromosome arms are concordant with their prognostic effects. In other words, whether the lower expression of a gene considered downregulated, and higher expression of a gene considered upregulated, are indicative of poorer prognosis among the colon cancer patients was investigated. This was done by generating Kaplan-Meier (KM) plots based entirely on the levels of expression (182 colon cancer samples were divided into two: high expression and low expression groups) of the dysregulated genes indicated in FIG. 12. Table 4 is a list of 59 dysregulated genes which satisfied the following criteria: a) the p-value (log rank or Wilcoxon) for KM is less than or equal to 0.05, and b) lower expression levels of downregulated genes, or higher expression levels of upregulated genes correlating to worse clinical outcome.

TABLE 4
Chromosomal Aberrations Corresponding to Expression Changes and Outcome

| | | | | Chromosomal | Class- | Accession | |
|---|---|---|---|---|---|---|---|
| Probe | Gene | | | Location_{—} | ifica- | # (Entrez | |
| Set ID | ID | AKA | Gene Title | MDB | tion | Gene ID) | Probe Sequence(s) |
| 202678_at | GTF2A2 | HsT18745, | general | chr15q22.2 | Loss_{—} | 2958 | AAGCCATGGCATATCAG |
| | | TF2A2, | transcription | | down | | TTATACAG |
| | | TFIIA | factor IIA, 2, | | | | (SEQ ID NO: 5379) |
| | | | 12 kDa | | | | TGGGAAACAGTCTTCAG |
| | | | | | | | GAGAGCCT |
| | | | | | | | (SEQ ID NO: 5380) |
| | | | | | | | TCATACAGTCTCAACAG |
| | | | | | | | ATCACCCC |
| | | | | | | | (SEQ ID NO: 5381) |
| | | | | | | | TTGCCCTTCAAGTTCTA |
| | | | | | | | CTTCAGTT |
| | | | | | | | (SEQ ID NO: 5382) |
| | | | | | | | TGCAGCACTGGCTCAGA |
| | | | | | | | GGGTCAGG |
| | | | | | | | (SEQ ID NO: 5383) |
| | | | | | | | TCAGGGGCTCTCTAAAT |
| | | | | | | | ACGTACAG |
| | | | | | | | (SEQ ID NO: 5384) |
| | | | | | | | GATTCTGCGATAATGTG |
| | | | | | | | TGGACTTT |
| | | | | | | | (SEQ ID NO: 5385) |
| | | | | | | | AATACTGGCTCCAATAC |
| | | | | | | | TACAGAAT |
| | | | | | | | (SEQ ID NO: 5386) |
| | | | | | | | CACCATCTTCTGTTATT |
| | | | | | | | CATTGCTT |
| | | | | | | | (SEQ ID NO: 5387) |
| | | | | | | | GAAACTTGTAACTCTGT |
| | | | | | | | AGCCTCTT |
| | | | | | | | (SEQ ID NO: 5388) |
| | | | | | | | CCTCTTACATCACCTTT |
| | | | | | | | ATTATACA |
| | | | | | | | (SEQ ID NO: 5389) |
| 219060_at | C8orf | FLJ10204 | chromosome 8 | chr8q24.13 | Gain_{—} | 55093 | AAGCTATCCTTTCATCG |
| | 32 | | open reading | | up | | AGGACAGC |
| | | | frame 32 | | | | (SEQ ID NO: 5390) |
| | | | | | | | ACATTATGGTACAGTTG |
| | | | | | | | GCTTGGAA |
| | | | | | | | (SEQ ID NO: 5391) |
| | | | | | | | GGCTTGGAATTATGTCT |
| | | | | | | | TTCTCTTT |
| | | | | | | | (SEQ ID NO: 5392) |
| | | | | | | | GTGCGTACTTGCTAAGAT |
| | | | | | | | ATTCCTG |
| | | | | | | | (SEQ ID NO: 5393) |
| | | | | | | | GATATTCCTGTGGCTCA |
| | | | | | | | TGCGTTAC |
| | | | | | | | (SEQ ID NO: 5394) |
| | | | | | | | GCGTTACAACACGAGGA |
| | | | | | | | CTTAAGCC |
| | | | | | | | (SEQ ID NO: 5395) |
| | | | | | | | AAGCCAGTAATCGTTTT |
| | | | | | | | TGTTCAGA |
| | | | | | | | (SEQ ID NO: 5396) |
| | | | | | | | AGAGGTGTGGAGGTAGA |
| | | | | | | | GCCAGCCC |
| | | | | | | | (SEQ ID NO: 5397) |
| | | | | | | | TGTCTCTCCAGCTACAT |
| | | | | | | | TGTAAGTT |
| | | | | | | | (SEQ ID NO: 5398) |
| | | | | | | | AAATGCCCATAAAAGGT |
| | | | | | | | GCCCATAA |
| | | | | | | | (SEQ ID NO: 5399) |
| | | | | | | | AGCCCTCTCAGACTTGA |
| | | | | | | | GCGTTAAT |
| 201950_x_at | CAPZB | RP4- | capping protein | chr1p36.1 | Loss_{—} | 832 | ACAAGTTGACCTCCACG |
| | | 657E11.7, | (actin filament) | | down | | GTGATGCT |
| | | CAPB, | muscle Z-line, | | | | (SEQ ID NO: 5401) |
| | | CAPPB, | beta | | | | GATGCTGTGGCTGCAGA |
| | | CAPZ | | | | | CCAACAAA |
| | | MGC104401, | | | | | (SEQ ID NO: 5402) |
| | | MGC129749, | | | | | GACCAACAAATCTGGCT |
| | | MGC129750 | | | | | CTGGCACC |
| | | | | | | | (SEQ ID NO: 5403) |
| | | | | | | | GGCACCATGAACCTCGG |
| | | | | | | | AGGCAGCC |
| | | | | | | | (SEQ ID NO: 5404) |
| | | | | | | | TGAAACTGTGAGTGACT |
| | | | | | | | GCTCCCCA |
| | | | | | | | (SEQ ID NO: 5405) |
| | | | | | | | TCCCCACACATAGCCAA |
| | | | | | | | CATCGGGC |
| | | | | | | | (SEQ ID NO: 5406) |
| | | | | | | | ACATCGGGCGCCTGGTA |
| | | | | | | | GAGGACAT |
| | | | | | | | (SEQ ID NO: 5407) |
| | | | | | | | CTGTTTCATGCTAACCA |
| | | | | | | | GACACGCC |
| | | | | | | | (SEQ ID NO: 5408) |
| | | | | | | | TGCACTCGTTAGATTCC |
| | | | | | | | TTTCTTAG |
| | | | | | | | (SEQ ID NO: 5409) |
| | | | | | | | AACAGCTGCATCATTGA |
| | | | | | | | CCGCACAG |
| | | | | | | | (SEQ ID NO: 5410) |
| | | | | | | | AGAATAAAGCCGATAGC |
| | | | | | | | CACCTCCT |
| | | | | | | | (SEQ ID NO: 5411) |
| 211595_s_at | MRPS11 | HCC-2; | mitochondrial | chr15q25 | Loss_{—} | 64963 | ACACAGATCCAGGTAGT |
| | | FLJ22512; | ribosomal | | down | | CTCTGCTA |
| | | FLJ23406 | protein S11 | | | | (SEQID NO: 5412) |
| | | | | | | | GTCTCTGCTAGTAATG |
| | | | | | | | AGCCCCTTG |
| | | | | | | | (SEQ ID NO: 5413) |
| | | | | | | | AGCACAGACAGCAGGCA |
| | | | | | | | TAGCCGCA |
| | | | | | | | (SEQ ID NO: 5414) |
| | | | | | | | AGCCGCAGCGGCGAGAG |
| | | | | | | | CTAAACAA |
| | | | | | | | (SEQ ID NO: 5415) |
| | | | | | | | CAAAAGGGCGTGATCCA |
| | | | | | | | CATCCGAG |
| | | | | | | | (SEQ ID NO: 5416) |
| | | | | | | | TTGTCTGCCATGCACGG |
| | | | | | | | ACTGATCA |
| | | | | | | | (SEQ ID NO: 5417) |
| | | | | | | | GGACTGATCATGGGCGG |
| | | | | | | | CCTGGAAG |
| | | | | | | | (SEQ ID NO: 5418) |
| | | | | | | | CTCAGCTCCAGTGGGAC |
| | | | | | | | CTTGTAAA |
| | | | | | | | (SEQ ID NO: 5419) |
| | | | | | | | GGACCTTGTAAAATGCT |
| | | | | | | | CCCTGTCA |
| | | | | | | | (SEQ ID NO: 5420) |
| | | | | | | | TCCCTGTCAGAGCTCTC |
| | | | | | | | CAGAATAT |
| | | | | | | | (SEQ ID NO: 5421) |
| | | | | | | | GGTGTGCCCCAGAAGTA |
| | | | | | | | AGCTTTGC |
| | | | | | | | (SEQ ID NO: 5422) |
| 212447_at | KBTBD2 | BKLHD1 | kelch repeat | chr7p14.3 | Gain_{—} | 25948 | ACAGGTCTTCCAGTCAG |
| | | | and BTB (POZ) | | up | | CTGGTCAT |
| | | | domain | | | | (SEQ ID NO: 5423) |
| | | | containing 2 | | | | CAGTAAGAATTGCCAGC |
| | | | | | | | CACTCATT |
| | | | | | | | (SEQ ID NO: 5424) |
| | | | | | | | CCACTCATTGTGCGGGT |
| | | | | | | | CTGCGTGG |
| | | | | | | | (SEQ ID NO: 5425) |
| | | | | | | | AAGCCTCCACTTTCTGT |
| | | | | | | | ATTATGTT |
| | | | | | | | (SEQ ID NO: 5426) |
| | | | | | | | GCTGATGTAGTTCTCCA |
| | | | | | | | TCTTCAAG |
| | | | | | | | (SEQ ID NO: 5427) |
| | | | | | | | CAGGAATTCTCTGCACT |
| | | | | | | | AGCTGTGC |
| | | | | | | | (SEQ ID NO: 5428) |
| | | | | | | | AACTAGCGCTGCTCTGG |
| | | | | | | | CATTAATC |
| | | | | | | | (SEQ ID NO: 5429) |
| | | | | | | | AATCCCAGGAACCACTA |
| | | | | | | | GCAGTAGT |
| | | | | | | | (SEQ ID NO: 5430) |
| | | | | | | | CGCCGCCAATCTAACAT |
| | | | | | | | GAGCACAG |
| | | | | | | | (SEQ ID NO: 5431) |
| | | | | | | | AGCACAGGTGCTTCATG |
| | | | | | | | ACAAACAT |
| | | | | | | | (SEQ ID NO: 5432) |
| | | | | | | | TCAACTGCACCATGTTC |
| | | | | | | | TGGCACTG |
| | | | | | | | (SEQ ID NO: 5433) |
| 202675_at | SDHB | FLJ92337, | succinate | chr1p36.1- | Loss_{—} | 6390 | ACCCTCTTCCACACATG |
| | | IP, PGL4, | dehydrogenase | p35 | down | | TATGTGAT |
| | | SDH, SDH1, | complex, | | | | (SEQ ID NO: 5434) |
| | | SDHIP | subunit B, | | | | AAAGGATCTTGTTCCC |
| | | | iron sulfur | | | | GATTTGAGC |
| | | | (Ip) | | | | (SEQ ID NO: 5435) |
| | | | | | | | GTTCCCGATTTGAGCAA |
| | | | | | | | CTTCTATG |
| | | | | | | | (SEQ ID NO: 5436) |
| | | | | | | | TTTGAGCAACTTCTATG |
| | | | | | | | CACAGTAC |
| | | | | | | | (SEQ ID NO: 5437) |
| | | | | | | | AGGATGAATCTCAGGAA |
| | | | | | | | GGCAAGCA |
| | | | | | | | (SEQ ID NO: 5438) |
| | | | | | | | GCAGTCCATAGAAGAGC |
| | | | | | | | GTGAGAAA |
| | | | | | | | (SEQ ID NO: 5439) |
| | | | | | | | AAGAGCGTGAGAAACTG |
| | | | | | | | GACGGGCT |
| | | | | | | | (SEQ ID NO: 5440) |
| | | | | | | | GGAACGGAGACAAATAT |
| | | | | | | | CTGGGGCC |
| | | | | | | | (SEQ ID NO: 5441) |
| | | | | | | | ATATCTGGGGCCTGCAG |
| | | | | | | | TTCTTATG |
| | | | | | | | (SEQ ID NO: 5442) |
| | | | | | | | CTGCAGTTCTTATGCAG |
| | | | | | | | GCCTATCG |
| | | | | | | | (SEQ ID NO: 5443) |
| | | | | | | | GATGACTTCACAGAGGA |
| | | | | | | | GCGCCTGG |
| | | | | | | | (SEQ ID NO: 5444) |
| 202838_at | FUCA1 | RP11- | fucosidase, | chr1p34 | Loss_{—} | 2517 | AGAAAGAGGCGCTGCTC |
| | | 45G17.1, | alpha-L-1, | | down | | ACTGTTTT |
| | | FUCA | tissue | | | | (SEQ ID NO: 5445) |
| | | | | | | | TAATCAACGAACTTCTC |
| | | | | | | | TTCTCCAC |
| | | | | | | | (SEQ ID NO: 5446) |
| | | | | | | | CTCCACCCAGAGATGGC |
| | | | | | | | TTTTCCAA |
| | | | | | | | (SEQ ID NO: 5447) |
| | | | | | | | ACAATGGAATCTTCCCT |
| | | | | | | | CTGTTCTC |
| | | | | | | | (SEQ ID NO: 5448) |
| | | | | | | | CTCTGATAACCTACTTG |
| | | | | | | | CTTACTCA |
| | | | | | | | (SEQ ID NO: 5449) |
| | | | | | | | CAATGCCTTTAAGCCAA |
| | | | | | | | GTCACCCT |
| | | | | | | | (SEQ ID NO: 5450) |
| | | | | | | | CCAAGTCACCCTGTTGC |
| | | | | | | | CTATGGGA |
| | | | | | | | (SEQ ID NO: 5451) |
| | | | | | | | GATTTGGCAAGCTCAAC |
| | | | | | | | CACATGCT |
| | | | | | | | (SEQ ID NO: 5452) |
| | | | | | | | GTTGTCACCAACAGTCT |
| | | | | | | | TTCTGCAA |
| | | | | | | | (SEQ ID NO: 5453) |
| | | | | | | | GGAAAAGGCTTACCAGG |
| | | | | | | | CTGCTATG |
| | | | | | | | (SEQ ID NO: 5454) |
| | | | | | | | GGCTGCTATGGTCAACT |
| | | | | | | | CTTCAGAA |
| | | | | | | | (SEQ ID NO: 5455) |
| 216510_x_at | EXOC7 | 2-5-3p, | immunoglobulin | chr17q25.1 | Loss_{—} | 28396/// | AGTGGGTCTCAGCTATT |
| | | DKFZp686J | heavy constant | | down | 3493/// | AGTGGGAG |
| | | 04253, | alpha 1/// | | | 3495/// | (SEQ ID NO: 5456) |
| | | EX070, | immunoglobulin | | | 3500/// | TCAGCTATTAGTGGGAG |
| | | EXO70, | heavy constant | | | 3507/// | TGCAGGTA |
| | | EXOC1, | delta/// | | | 3509 | (SEQ ID NO: 5457) |
| | | Exo70p, | immunoglobulin | | | | GGAGTGCAGGTACCACA |
| | | FLJ40965, | heavy constant | | | | TACTACGC |
| | | FLJ46415, | gamma 1 (G1m | | | | (SEQ ID NO: 5458) |
| | | YJL085W | marker)/// | | | | CAGGTACCACATACTAC |
| | | | immunoglobulin | | | | GCAGACTC |
| | | | heavy constant | | | | (SEQ ID NO: 5459) |
| | | | mu/// | | | | TACGCAGACTCCGTGAA |
| | | | immunoglobulin | | | | GGGCCGGT |
| | | | heavy variable | | | | (SEQ ID NO: 5460) |
| | | | group/// | | | | TTCACCACCTCCAGAGA |
| | | | immunoglobulin | | | | CAATTCCA |
| | | | heavy variable | | | | (SEQ ID NO: 5461) |
| | | | 4-31 | | | | GACAATTCCAAGAACAC |
| | | | | | | | GCTGTATC |
| | | | | | | | (SEQ ID NO: 5462) |
| | | | | | | | CAAGAACACGCTGTATC |
| | | | | | | | TGCAAATG |
| | | | | | | | (SEQ ID NO: 5463) |
| | | | | | | | CTGAGAGCCGAGGACAC |
| | | | | | | | GGCCGTAT |
| | | | | | | | (SEQ ID NO: 5464) |
| | | | | | | | GACACGGCCGTATATTA |
| | | | | | | | CTGTGCGA |
| | | | | | | | (SEQ ID NO: 5465) |
| | | | | | | | GCCGTATATTACTGTGC |
| | | | | | | | GAAATCCC |
| | | | | | | | (SEQ ID NO: 5466) |
| 221774_x_at | FAM48A | C13; | family with | chr13q13.3 | Gain_{—} | 55578 | ATACTTCAAGTCTCAGG |
| | | FP757; | sequence | | up | | CCCTTAAA |
| | | P38IP; | similarity 48, | | | | (SEQ ID NO: 5467) |
| | | C13orf19; | member A | | | | CTTAAATCTACTCCAG |
| | | bA421P11.4 | | | | | CTTCCAGGT |
| | | | | | | | (SEQ ID NO: 5468) |
| | | | | | | | TATTTTTAACACTCTG |
| | | | | | | | CAGCAGCAG |
| | | | | | | | (SEQ ID NO: 5469) |
| | | | | | | | GTAGTCCTCAACAGCCA |
| | | | | | | | GGGGAGCA |
| | | | | | | | (SEQ ID NO: 5470) |
| | | | | | | | CAAGAACAGGCCTTATC |
| | | | | | | | TGCTCAGC |
| | | | | | | | (SEQ ID NO: 5471) |
| | | | | | | | CAGTCACAGGCAGCTGC |
| | | | | | | | AGTTGCGA |
| | | | | | | | (SEQ ID NO: 5472) |
| | | | | | | | GCAGTTGCGATTCTTGC |
| | | | | | | | AGCATCAA |
| | | | | | | | (SEQ ID NO: 5473) |
| | | | | | | | GCAGCAGCAGCACAAAC |
| | | | | | | | AGCTCAGC |
| | | | | | | | (SEQ ID NO: 5474) |
| | | | | | | | AGCTCAGCTACATCATC |
| | | | | | | | ATCGGCAT |
| | | | | | | | (SEQ ID NO: 5475) |
| | | | | | | | TCATCGGCATACAGGCA |
| | | | | | | | GCCAGTCA |
| | | | | | | | (SEQ ID NO: 5476) |
| | | | | | | | GTCTTGCATTACTTTTT |
| | | | | | | | GTTCCTTT |
| | | | | | | | (SEQ IDNO: 5477) |
| 200027_at | NARS | ASNRS, | asparaginyl- | chr18q21.2- | Loss_{—} | 4677 | ATCTTTTCTAACTCTGC |
| | | NARS1 | tRNA | q21.3 | down | | TTAGCTGC |
| | | | synthetase | | | | (SEQ ID NO: 5478) |
| | | | | | | | AGCTGCTAATAATCCTG |
| | | | | | | | AGGCATAG |
| | | | | | | | (SEQ ID NO: 5479) |
| | | | | | | | TAGTGATTCACAGTATC |
| | | | | | | | CCTCTTAG |
| | | | | | | | (SEQ ID NO: 5480) |
| | | | | | | | AGTATCCCTCTTAGCAT |
| | | | | | | | TAATTTAA |
| | | | | | | | (SEQ ID NO: 5481) |
| | | | | | | | GAGGCAGATTGATITTC |
| | | | | | | | CCTCTTTC |
| | | | | | | | (SEQ ID NO: 5482) |
| | | | | | | | CCCACCATGTCCTTAGA |
| | | | | | | | TCTAATCT |
| | | | | | | | (SEQ ID NO: 5483) |
| | | | | | | | TAGATCTAATCTGTGCT |
| | | | | | | | ACCTTATT |
| | | | | | | | (SEQ ID NO: 5484) |
| | | | | | | | GCTACCTTATTAACTCA |
| | | | | | | | CAGCAGGC |
| | | | | | | | (SEQ ID NO: 5485) |
| | | | | | | | ACAGCAGGCTTACTGAA |
| | | | | | | | TGGCTTCA |
| | | | | | | | (SEQ ID NO: 5486) |
| | | | | | | | CAGATTTAGTTGATTTC |
| | | | | | | | TCCACCAA |
| | | | | | | | (SEQ ID NO: 5487) |
| | | | | | | | GCATGTCATGTATTCTC |
| | | | | | | | AATAGGCT |
| | | | | | | | (SEQ ID NO: 5488) |
| 208540_x_at | LOC | S100A11P | hypothetical | chr7q22.1 | Gain_{—} | 729659 | ATGGCAAAAATCTCCAG |
| | 729659 | | LOC729659 | | up | | CCCTACAG |
| | | | | | | | (SEQ ID NO: 5489) |
| | | | | | | | CTCCAGCCCTACAGAGA |
| | | | | | | | CTGAGCGG |
| | | | | | | | (SEQ ID NO: 5490) |
| | | | | | | | GACTGAGCGGTGCATTG |
| | | | | | | | AGTCCCTG |
| | | | | | | | (SEQ ID NO: 5491) |
| | | | | | | | AGTCCCTGATTGCTGTT |
| | | | | | | | TTCCAGAA |
| | | | | | | | (SEQ ID NO: 5492) |
| | | | | | | | GGATGGTTACAACCGCA |
| | | | | | | | ATCTCTCC |
| | | | | | | | (SEQ ID NO: 5493) |
| | | | | | | | CAACCGCAATCTCTCCA |
| | | | | | | | AGACGGAG |
| | | | | | | | (SEQ ID NO: 5494) |
| | | | | | | | GACGGAGTTCCTAAGCT |
| | | | | | | | TCATGAAT |
| | | | | | | | (SEQ ID NO: 5495) |
| | | | | | | | GAGCTGGCTGCCTTTAC |
| | | | | | | | AAAGAACC |
| | | | | | | | (SEQ ID NO: 5496) |
| | | | | | | | CCGGTGTCCTTGACCAC |
| | | | | | | | ATGAAGAA |
| | | | | | | | (SEQ ID NO: 5497) |
| | | | | | | | GATGTCAGCAGTGATGG |
| | | | | | | | GCAGTTAG |
| | | | | | | | (SEQ ID NO: 5498) |
| | | | | | | | AATCTGATTGGTGGCCT |
| | | | | | | | AGCTGTGG |
| | | | | | | | (SEQ ID NO: 5499) |
| 208813_at | GOT1 | GIG18 | glutamic- | chr10q24.1- | Loss_{—} | 2805 | ATGGCTGACCGGATTCT |
| | | | oxaloacetic | q25.1 | down | | GACCATGA |
| | | | transaminase 1, | | | | (SEQ ID NO: 5500) |
| | | | soluble | | | | GGCACGACTAGAAGCCC |
| | | | (aspartate | | | | TCAAAACC |
| | | | amino- | | | | (SEQ ID NO: 5501) |
| | | | transferase | | | | AAATTGGCATGTTCAG |
| | | | 1) | | | | CTTCACTGG |
| | | | | | | | (SEQ ID NO: 5502) |
| | | | | | | | GCTGCCAAGTGGTCGAA |
| | | | | | | | TCAACGTG |
| | | | | | | | (SEQ ID NO: 5503) |
| | | | | | | | CTCCATCCATGAAGCAG |
| | | | | | | | TCACCAAA |
| | | | | | | | (SEQ ID NO: 5504) |
| | | | | | | | TCCAGTGAAGAAACACC |
| | | | | | | | ACCCGTCC |
| | | | | | | | (SEQ ID NO: 5505) |
| | | | | | | | GTCCAGTACCACCAAAG |
| | | | | | | | TAGTTCTC |
| | | | | | | | (SEQ ID NO: 5506) |
| | | | | | | | CTGCCTGCACAAACCTA |
| | | | | | | | CATGTACA |
| | | | | | | | (SEQ ID NO: 5507) |
| | | | | | | | GACTGAAAGGCTGCTCT |
| | | | | | | | GGTGAGGC |
| | | | | | | | (SEQ ID NO: 5508) |
| | | | | | | | TAAACCGGCCCCACATG |
| | | | | | | | AAGAGAAC |
| | | | | | | | (SEQ ID NO: 5509) |
| | | | | | | | GAGAACATCCCTTGAGA |
| | | | | | | | CGAATTTG |
| | | | | | | | (SEQ ID NO: 5510) |
| 218371_s_at | PSPC1 | RP11- | paraspeckle | chr13q12.11 | Gain_{—} | 55269 | ATGTGGCTGAAGTTACC |
| | | 523H24.2, | component 1 | | up | | CGAAGTTC |
| | | DKFZp566B | | | | | (SEQ ID NO: 5511) |
| | | 1447, | | | | | GTTACCCGAAGTTCAGC |
| | | FLJ10955, | | | | | TTGCAGTG |
| | | PSP1 | | | | | (SEQ ID NO: 5512) |
| | | | | | | | AGTTCAGCTTGCAGTGT |
| | | | | | | | AATTCAGA |
| | | | | | | | (SEQ ID NO: 5513) |
| | | | | | | | GAATCATACTTCCTTTC |
| | | | | | | | AGGTCAAA |
| | | | | | | | (SEQ ID NO: 5514) |
| | | | | | | | GTGGGAAATATCCTGTT |
| | | | | | | | CGTGACCT |
| | | | | | | | (SEQ ID NO: 5515) |
| | | | | | | | TCCTGTTCGTGACCTAG |
| | | | | | | | GAGAAACA |
| | | | | | | | (SEQ ID NO: 5516) |
| | | | | | | | TTCTTACTCCATACAAG |
| | | | | | | | CAGTTTAT |
| | | | | | | | (SEQ ID NO: 5517) |
| | | | | | | | AAACATGCTACAGGGGC |
| | | | | | | | ACATTTTC |
| | | | | | | | (SEQ ID NO: 5518) |
| | | | | | | | ACAGGGGCACATTTTCT |
| | | | | | | | GTGGTTAA |
| | | | | | | | (SEQ ID NO: 5519) |
| | | | | | | | GCAGTTTTCCTTTGCAT |
| | | | | | | | TGAGGGTT |
| | | | | | | | (SEQ ID NO: 5520) |
| | | | | | | | CTTTGCATTGAGGGTTT |
| | | | | | | | TGGCTTTT |
| | | | | | | | (SEQ ID NO: 5521) |
| 202962_at | KIF13B | GAKIN, | kinesin family | chr8p21.1 | Loss_{—} | 23303 | ATGTTTCTGGGCCTACC |
| | | KIAA0639 | member 13B | | down | | AAGGTTCA |
| | | | | | | | (SEQ ID NO: 5522) |
| | | | | | | | CTGGGAATGTGACTGCT |
| | | | | | | | GGAGCCTG |
| | | | | | | | (SEQ ID NO: 5523) |
| | | | | | | | TGAGAGTGTGTGTTGGT |
| | | | | | | | GGCTGTGC |
| | | | | | | | (SEQ ID NO: 5524) |
| | | | | | | | ACAAGACGGGAGTCACC |
| | | | | | | | CTGTGCTT |
| | | | | | | | (SEQ ID NO: 5525) |
| | | | | | | | CCAAGATACTGACCCAT |
| | | | | | | | TGAACCCC |
| | | | | | | | (SEQ ID NO: 5526) |
| | | | | | | | TTCTCTCCACAAAGTCC |
| | | | | | | | GTGGTGCC |
| | | | | | | | (SEQ ID NO: 5527) |
| | | | | | | | GGTGGGCTGCAGACACT |
| | | | | | | | AATGGTGT |
| | | | | | | | (SEQ ID NO: 5528) |
| | | | | | | | GGTCTTGGAACAGCTTC |
| | | | | | | | TCTATGTG |
| | | | | | | | (SEQ ID NO: 5529) |
| | | | | | | | GCTTCTCTATGTGTGGA |
| | | | | | | | TTCGTGTA |
| | | | | | | | (SEQ ID NO: 5530) |
| | | | | | | | AACCACCTGTAATTTTG |
| | | | | | | | TACCATAT |
| | | | | | | | (SEQ ID NO: 5531) |
| | | | | | | | GTACCATATGTGTCTTC |
| | | | | | | | CATCAAA |
| | | | | | | | (SEQ ID NO: 5532) |
| 212166_at | XPO7 | KIAA0745, | exportin 7 | chr8p21 | Loss_{—} | 23039 | ATTCCTGTTACTGTATT |
| | | RANBP16 | | | down | | AACCCTTG |
| | | | | | | | (SEQ ID NO: 5533) |
| | | | | | | | GGAACTCTAAGCCATGC |
| | | | | | | | CAGAACAC |
| | | | | | | | (SEQ ID NO: 5534) |
| | | | | | | | CCCTAGCCCCTTGCAGT |
| | | | | | | | GATAAATA |
| | | | | | | | (SEQ ID NO: 5535) |
| | | | | | | | GTGTTTGGTGTTCTTAT |
| | | | | | | | CTCCACCC |
| | | | | | | | (SEQ ID NO: 5536) |
| | | | | | | | TCAGACAGATGCCTCTT |
| | | | | | | | GCTTTTAA |
| | | | | | | | (SEQ ID NO: 5537) |
| | | | | | | | ACGTGTTGTAGGGTTCT |
| | | | | | | | TGGTCTGT |
| | | | | | | | (SEQ ID NO: 5538) |
| | | | | | | | GAGAAGGAAGTGAGCCC |
| | | | | | | | ACTGCTCT |
| | | | | | | | (SEQ ID NO: 5539) |
| | | | | | | | ATGTGGGTGAGTCCACC |
| | | | | | | | AGAGGCCC |
| | | | | | | | (SEQ ID NO: 5540) |
| | | | | | | | CCAGCCCTCGAGGCAGT |
| | | | | | | | GTGTGTGG |
| | | | | | | | (SEQ ID NO: 5541) |
| | | | | | | | ATATGTACCCTGCACTC |
| | | | | | | | ATGAATGT |
| | | | | | | | (SEQ ID NO: 5542) |
| | | | | | | | GAAGTATTTAACATTCT |
| | | | | | | | CCCATCCC |
| | | | | | | | (SEQ ID NO: 5543) |
| 217962_at | NOLA3 | MGC70651, | nucleolar protein | chr15q14- | Loss_{—} | 55505 | ATTGACGAACACGTGAC |
| | | NOP10, | family A, member | q15 | down | | GCGGTCGG |
| | | NOP10P | 3 (H/ACA small | | | | (SEQ ID NO: 5544) |
| | | | nucleolar RNPs) | | | | CGGACCACTGCAGACTG |
| | | | | | | | AGCGGTGG |
| | | | | | | | (SEQ ID NO: 5545) |
| | | | | | | | GATCATGTTCTCCAGTA |
| | | | | | | | TTACCTC |
| | | | | | | | (SEQ ID NO: 5546) |
| | | | | | | | TGACCCGATGGGACAAC |
| | | | | | | | AGACCTGC |
| | | | | | | | (SEQ ID NO: 5547) |
| | | | | | | | GGTTCTCCCCAGATGAC |
| | | | | | | | AAATACTC |
| | | | | | | | (SEQ ID NO: 5548) |
| | | | | | | | TCTCGACACCGAATCAC |
| | | | | | | | CATCAAGA |
| | | | | | | | (SEQ ID NO: 5549) |
| | | | | | | | GCTTCAAGGTGCTCATG |
| | | | | | | | ACCCAGCA |
| | | | | | | | (SEQ ID NO: 5550) |
| | | | | | | | TGAGGGTCCCTTAAACT |
| | | | | | | | GATGTCTT |
| | | | | | | | (SEQ ID NO: 5551) |
| | | | | | | | GAGACTCCGTAACCAAA |
| | | | | | | | CTCTTCGG |
| | | | | | | | (SEQ ID NO: 5552) |
| | | | | | | | ACCAAACTCTTCGGACT |
| | | | | | | | GTGAGCCC |
| | | | | | | | (SEQ ID NO: 5553) |
| | | | | | | | GCCATACTCTTTGGCAT |
| | | | | | | | CCAGTCTC |
| | | | | | | | (SEQ ID NO: 5554) |
| 218273_s_at | PPM2C | FLJ32517, | protein | chr8q22.1 | Gain_{—} | 54704 | ATTGACTTTGCAGCAGG |
| | | MGC119646, | phosphatase 2C, | | up | | GTGGCAGG |
| | | PDH, PDP, | magnesium- | | | | (SEQ ID NO: 5555) |
| | | PDP1, | dependent, | | | | GTCCTGCCTAGCTCAGA |
| | | PDPC | catalytic | | | | TTTCATGG |
| | | | subunit | | | | (SEQ ID NO: 5556) |
| | | | | | | | CAGATTTCATGGCACCT |
| | | | | | | | GCACTTGA |
| | | | | | | | (SEQ ID NO: 5557) |
| | | | | | | | CACTTCTTTATCACAGG |
| | | | | | | | TGTCTTGA |
| | | | | | | | (SEQ ID NO: 5558) |
| | | | | | | | TAGCTTCTTTTACCAAC |
| | | | | | | | CTGAGAAA |
| | | | | | | | (SEQ ID NO: 5559) |
| | | | | | | | GTATCTTGCTGTGTGTA |
| | | | | | | | GTCTCTTG |
| | | | | | | | (SEQ ID NO: 5560) |
| | | | | | | | TCACACCTTTCTTCACT |
| | | | | | | | GAGATTCC |
| | | | | | | | (SEQ ID NO: 5561) |
| | | | | | | | TCCAGTTCTTCAAGCCA |
| | | | | | | | TAAATGAC |
| | | | | | | | (SEQ ID NO: 5562) |
| | | | | | | | GATTATTTGACTGGAAT |
| | | | | | | | GCTTCTTA |
| | | | | | | | (SEQ ID NO: 5563) |
| | | | | | | | GAATAACTATACTCCGT |
| | | | | | | | TATCCACC |
| | | | | | | | (SEQ ID NO: 5564) |
| | | | | | | | TATCCACCCGATTTCCT |
| | | | | | | | AATGTAAT |
| | | | | | | | (SEQ ID NO: 5565) |
| 200847_s_at | TMEM66 | FLJ22274, | trans- | chr8p12 | Loss_{—} | 51669 | ATTTCTAAAACCTGTGA |
| | | FOAP-7, | protein 66 | | down | | TGCCCTAA |
| | | HSPC035, | | | | | (SEQ ID NO: 5566) |
| | | MGC8721, | | | | | AGGTGGTTGTAGCTGAT |
| | | XTP3 | | | | | GAGTTATT |
| | | | | | | | (SEQ ID NO: 5567) |
| | | | | | | | TTATGCTAATTTGTGAG |
| | | | | | | | TTCTGATC |
| | | | | | | | (SEQ ID NO: 5568) |
| | | | | | | | TCTGATCACTTTTGAGC |
| | | | | | | | TCTGAAGC |
| | | | | | | | (SEQ ID NO: 5569) |
| | | | | | | | GAGCTCTGAAGCTTTGA |
| | | | | | | | ATCATTCA |
| | | | | | | | (SEQ ID NO: 5570) |
| | | | | | | | TCAGTGGTGGAGATGGC |
| | | | | | | | CTTCTGGT |
| | | | | | | | (SEQ ID NO: 5571) |
| | | | | | | | GGAGATGGCCTTCTGGT |
| | | | | | | | AACTGAAT |
| | | | | | | | (SEQ ID NO: 5572) |
| | | | | | | | TAACTGAATATTACCTT |
| | | | | | | | CTGTAGGA |
| | | | | | | | (SEQ ID NO: 5573) |
| | | | | | | | TGTGAATGACTCTGTGC |
| | | | | | | | TGGCAAAA |
| | | | | | | | (SEQ ID NO: 5574) |
| | | | | | | | AAACCTCTATATTTCTT |
| | | | | | | | TCGTTCAT |
| | | | | | | | (SEQ ID NO: 5575) |
| | | | | | | | TAACAAAAGCATGCAGT |
| | | | | | | | TCTCTGTG |
| | | | | | | | (SEQ ID NO: 5576) |
| 209374_s_at | IGHM | DKFZp686I | immunoglobulin | chr14q32.33 | Loss_{—} | 3507 | CACCATCACGTGCCTGG |
| | | 15196, | heavy constant | | down | | TGACGGGC |
| | | DKFZp686I | mu | | | | (SEQ ID NO: 5577) |
| | | 15212, | | | | | GACGTCTTCGTGCAGTG |
| | | FLJ00385, | | | | | GATGCAGA |
| | | MGC104996, | | | | | (SEQ ID NO: 5578) |
| | | MGC52291, | | | | | GCAGTGGATGCAGAGGG |
| | | MU, VH | | | | | GGCAGCCC |
| | | | | | | | (SEQ ID NO: 5579) |
| | | | | | | | GATGCAGAGGGGGCAGC |
| | | | | | | | CCTTGTCC |
| | | | | | | | (SEQ ID NO: 5580) |
| | | | | | | | GCCCTTGTCCCCGGAGA |
| | | | | | | | AGTATGTG |
| | | | | | | | (SEQ ID NO: 5581) |
| | | | | | | | CCGGAGAAGTATGTGAC |
| | | | | | | | CAGCGCCC |
| | | | | | | | (SEQ ID NO: 5582) |
| | | | | | | | CATCCTGACCGTGTCCG |
| | | | | | | | AAGAGGAA |
| | | | | | | | (SEQ ID NO: 5583) |
| | | | | | | | GACCGTGTCCGAAGAGG |
| | | | | | | | AATGGAAC |
| | | | | | | | (SEQ ID NO: 5584) |
| | | | | | | | TGCCCAACAGGGTCACC |
| | | | | | | | GAGAGGAC |
| | | | | | | | (SEQ ID NO: 5585) |
| | | | | | | | GTCACCGAGAGGACCGT |
| | | | | | | | GGACAAGT |
| | | | | | | | (SEQ ID NO: 5586) |
| | | | | | | | CGAGAGGACCGTGGACA |
| | | | | | | | AGTCCACC |
| | | | | | | | (SEQ ID NO: 5587) |
| 54970_at | ZMIZ2 | DKFZp761I | zinc finger, | chr7p13 | Gain_{—} | 83637 | CAGCTTCAAGCAGAGG |
| | | 2123, | MIZ-type | | up | | CCTCGGGGT |
| | | KIAA1886, | containing 2 | | | | (SEQ ID NO: 5588) |
| | | ZIMP7, | | | | | TGTAGAAACACTAACAG |
| | | hZIMP7 | | | | | CTGGGAGA |
| | | | | | | | (SEQ ID NO: 5589) |
| | | | | | | | ATCAGGGAGGAACCTCC |
| | | | | | | | AATGGGTG |
| | | | | | | | (SEQ ID NO: 5590) |
| | | | | | | | AGGAGGAAGCCACTGCA |
| | | | | | | | TCTGTTGG |
| | | | | | | | (SEQ ID NO: 5591) |
| | | | | | | | GCATCTGTTGGCTCAGG |
| | | | | | | | GCCCCAGC |
| | | | | | | | (SEQ ID NO: 5592) |
| | | | | | | | CGCCTGGGCTGTTGTGT |
| | | | | | | | CTCCTGTC |
| | | | | | | | (SEQ ID NO: 5593) |
| | | | | | | | CTGTTGTGTCTCCTGTC |
| | | | | | | | TGTGCCGA |
| | | | | | | | (SEQ ID NO: 5594) |
| | | | | | | | GTTGTGTCTCCTGTCTG |
| | | | | | | | TGCCGATC |
| | | | | | | | (SEQ ID NO: 5595) |
| | | | | | | | TGTCTCCTGTCTGTGCC |
| | | | | | | | GATCTCTA |
| | | | | | | | (SEQ ID NO: 5596) |
| | | | | | | | CTGTCTGTGCCGATCTC |
| | | | | | | | TATTAAAG |
| | | | | | | | (SEQ ID NO: 5597) |
| | | | | | | | GTCTGTGCCGATCTCTA |
| | | | | | | | TTAAAGGA |
| | | | | | | | (SEQ ID NO: 5598) |
| | | | | | | | CTGTGCCGATCTCTATT |
| | | | | | | | AAAGGACT |
| | | | | | | | (SEQ ID NO: 5599) |
| | | | | | | | GTGCCGATCTCTATTAA |
| | | | | | | | AGGACTCC |
| | | | | | | | (SEQ ID NO: 5600) |
| | | | | | | | GCCGATCTCTATTAAAG |
| | | | | | | | GACTCCCT |
| | | | | | | | (SEQ ID NO: 5601) |
| | | | | | | | CTCTATTAAAGGACTCC |
| | | | | | | | CTCTTGGT |
| | | | | | | | (SEQ ID NO: 5602) |
| | | | | | | | CTATTAAAGGACTCCCT |
| | | | | | | | CTTGGTGG |
| | | | | | | | (SEQ ID NO: 5603) |
| 200611_s_at | WDR1 | AIP1, | WD repeat | chr4p16.1 | Loss_{—} | 9948 | CATGCAAAAATCGTCTG |
| | | NORI-1 | domain 1 | | down | | CCTGGCCT |
| | | | | | | | (SEQ ID NO: 5604) |
| | | | | | | | TGGCCTGGTCCCCAGAC |
| | | | | | | | AATGAACA |
| | | | | | | | (SEQ ID NO: 5605) |
| | | | | | | | AGACAATGAACACTTTG |
| | | | | | | | CCTCCGGT |
| | | | | | | | (SEQ ID NO: 5606) |
| | | | | | | | ACTTTGCCTCCGGTGGC |
| | | | | | | | ATGGACAT |
| | | | | | | | (SEQ ID NO: 5607) |
| | | | | | | | GTGTATGTTTGGACCCT |
| | | | | | | | GAGTGACC |
| | | | | | | | (SEQ ID NO: 5608 |
| | | | | | | | AAGATCCAAGATGCAC |
| | | | | | | | ACCGGCTGC |
| | | | | | | | (SEQ ID NO: 5609) |
| | | | | | | | ATGATGCCTCTGTCAAG |
| | | | | | | | GAGTGGAC |
| | | | | | | | (SEQ ID NO: 5610) |
| | | | | | | | AGGAGTGGACAATCACC |
| | | | | | | | TACTGAGG |
| | | | | | | | (SEQ ID NO: 5611) |
| | | | | | | | CCGCCTCTGGATGGACC |
| | | | | | | | GAATCAGG |
| | | | | | | | (SEQ ID NO: 5612) |
| | | | | | | | AACTGCAGCGGAACATG |
| | | | | | | | TCATTTCT |
| | | | | | | | (SEQ ID NO: 5613) |
| | | | | | | | TCGTCTCTGCAGGGTGT |
| | | | | | | | CTGTACAC |
| | | | | | | | (SEQ ID NO: 5614) |
| 210154_at | ME2 | 0 | malic enzyme 2, | chr6p25- | Loss_{—} | 4200 | CCAGGTGTGGCTTTAGC |
| | | | NAD(+)- | p24\|18q21 | down | | TGTTATTC |
| | | | dependent, | | | | (SEQ ID NO: 5615) |
| | | | mitochondrial | | | | GTGGCTTTAGCTGTTAT |
| | | | | | | | TCTCTGTA |
| | | | | | | | (SEQ ID NO: 5616) |
| | | | | | | | TATTCTCTGTAACACCC |
| | | | | | | | GGCATATT |
| | | | | | | | (SEQ ID NO: 5617) |
| | | | | | | | ACCCGGCATATTAGTGA |
| | | | | | | | CAGTGTTT |
| | | | | | | | (SEQ ID NO: 5618) |
| | | | | | | | GACAGTGTTTTCCTAGA |
| | | | | | | | AGCTGCAA |
| | | | | | | | (SEQ ID NO: 5619) |
| | | | | | | | AGCTGCAAAGGCCCTGA |
| | | | | | | | CAAGCCAA |
| | | | | | | | (SEQ ID NO: 5620) |
| | | | | | | | AGGCCCTGACAAGCCAA |
| | | | | | | | TTGACAGA |
| | | | | | | | (SEQ ID NO: 5621) |
| | | | | | | | AATTGACAGATGAAGAG |
| | | | | | | | CTAGCCCA |
| | | | | | | | (SEQ ID NO: 5622) |
| | | | | | | | GCTAGCCCAAGGGAGAC |
| | | | | | | | TTTACCCA |
| | | | | | | | (SEQ ID NO: 5623) |
| | | | | | | | TACCCACCGCTTGCTAA |
| | | | | | | | TATTCAGG |
| | | | | | | | (SEQ ID NO: 5624) |
| | | | | | | | GGAAGTTTCTATTAACA |
| | | | | | | | TTGCTATT |
| | | | | | | | (SEQ ID NO: 5625) |
| 217875_s_at | TMEPAI | PMEPA1, | prostate | chr20q13. | Gain_{—} | 56937 | CCCCAAACAGAACCATC |
| | | STAG1 | trans- | 31-q13.33 | up | | TTCGACAG |
| | | | membrane | | | | (SEQ ID NO: 5626) |
| | | | protein, | | | | ATCTTCGACAGTGACCT |
| | | | androgen | | | | GATGGATA |
| | | | induced 1 | | | | (SEQ ID NO: 5627) |
| | | | | | | | GATGGATAGTGCCAGGC |
| | | | | | | | TGGGCGGC |
| | | | | | | | (SEQ ID NO: 5628) |
| | | | | | | | CGGCGGGCGCATGGAGG |
| | | | | | | | GGCCGCCG |
| | | | | | | | (SEQ ID NO: 5629) |
| | | | | | | | CCTAGAGAGCGCAGCCA |
| | | | | | | | TCTGGAGC |
| | | | | | | | (SEQ ID NO: 5630) |
| | | | | | | | AACAGAAAGGACACCCT |
| | | | | | | | CTCTAGGG |
| | | | | | | | (SEQ ID NO: 5631) |
| | | | | | | | GGCTGGGGCTGCGTAGG |
| | | | | | | | TGAAAAGG |
| | | | | | | | (SEQ ID NO: 5632) |
| | | | | | | | TGAAAAGGCAGAACACT |
| | | | | | | | CCGCGCTT |
| | | | | | | | (SEQ ID NO: 5633) |
| | | | | | | | GTGAGAGGAAGGCGGGG |
| | | | | | | | GGCGCAGC |
| | | | | | | | (SEQ ID NO: 5634) |
| | | | | | | | AAACCACGTTTCTTTGT |
| | | | | | | | TGAGCTGT |
| | | | | | | | (SEQ ID NO: 5635) |
| | | | | | | | GTTGAGCTGTGTCTTGA |
| | | | | | | | AGGCAAAA |
| | | | | | | | (SEQ ID NO: 5636) |
| 218435_at | DNAJC15 | MCJ; | DnaJ (Hsp40) | chr13q14.1 | Gain_{—} | 29103 | CCGACGTCGACCAGCAG |
| | | HSD18; | homolog, | | up | | GGACTGGT |
| | | DNAJD1 | subfamily C, | | | | (SEQ ID NO: 5637) |
| | | | member 15 | | | | GATAGCTGTAGGACTGG |
| | | | | | | | GTGTTGCA |
| | | | | | | | (SEQ ID NO: 5638) |
| | | | | | | | GGTGTTGCAGCTCTTGC |
| | | | | | | | ATTTGCAG |
| | | | | | | | (SEQ ID NO: 5639) |
| | | | | | | | GCATTTGCAGGTCGCTA |
| | | | | | | | CGCATTTC |
| | | | | | | | (SEQ ID NO: 5640) |
| | | | | | | | ACGCATTTCGGATCTGG |
| | | | | | | | AAACCTCT |
| | | | | | | | (SEQ ID NO: 5641) |
| | | | | | | | GAAGATTTCAACTCCTA |
| | | | | | | | GCTTTTCA |
| | | | | | | | (SEQ ID NO: 5642) |
| | | | | | | | TTTAGGTGTAAGCCCAT |
| | | | | | | | CTGCTGGC |
| | | | | | | | (SEQ ID NO: 5643) |
| | | | | | | | TGGATCTCCTTACGTAG |
| | | | | | | | CAGCCAAA |
| | | | | | | | (SEQ ID NO: 5644) |
| | | | | | | | ACATGGTCTTCTTAATT |
| | | | | | | | TTCTATAT |
| | | | | | | | (SEQ ID NO: 5645) |
| | | | | | | | TGGATTGACCACAGTCT |
| | | | | | | | TATCTTCC |
| | | | | | | | (SEQ ID NO: 5646) |
| | | | | | | | GTCTTATCTTCCACCAT |
| | | | | | | | TAAGCTGT |
| | | | | | | | (SEQ ID NO: 5647) |
| 213738_s_at | ATP5A1 | ATP5A, | ATP synthase, | chr18q12- | Loss_{—} | 498 | CGCCCTGCAATTAACGT |
| | | ATP5AL2, | H+ | q21 | down | | TGGTCTGT |
| | | ATPM, | transporting, | | | | (SEQ ID NO: 5648) |
| | | MOM2, | mitochondrial | | | | TCCGCTGCCCAAACCAG |
| | | OMR, ORM, | F1 complex, | | | | GGCTATGA |
| | | hATP1 | alpha | | | | (SEQ ID NO: 5649) |
| | | | subunit 1, | | | | GAAGCTGGAATTGGCT |
| | | | cardiac muscle | | | | CAGTATCGT |
| | | | | | | | (SEQ ID NO: 5650) |
| | | | | | | | GTATCGTGAGGTTGCTG |
| | | | | | | | CTTTTGCC |
| | | | | | | | (SEQ ID NO: 5651) |
| | | | | | | | AACTTTTGAGTCGTGGC |
| | | | | | | | GTGCGTCT |
| | | | | | | | (SEQ ID NO: 5652) |
| | | | | | | | GGCGTGCGTCTAACTGA |
| | | | | | | | GTTGCTGA |
| | | | | | | | (SEQ ID NO: 5653) |
| | | | | | | | CAGTATTCTCCCATGGC |
| | | | | | | | TATTGAAG |
| | | | | | | | (SEQ ID NO: 5654) |
| | | | | | | | AAGTGGCTGTTATCTAT |
| | | | | | | | GCGGGTGT |
| | | | | | | | (SEQ ID NO: 5655) |
| | | | | | | | GCACCAAGCCTTGTTGG |
| | | | | | | | GCACTATC |
| | | | | | | | (SEQ ID NO: 5656) |
| | | | | | | | AGCTTAAACTCCTGTGG |
| | | | | | | | ATTCACAT |
| | | | | | | | (SEQ ID NO: 5657) |
| | | | | | | | GTTCAGTTTTGTCATTG |
| | | | | | | | TTCTAGTA |
| | | | | | | | (SEQ ID NO: 5658) |
| 206134_at | ADAMDE | M12.219 | ADAM-like, | chr8p21.2 | Loss_{—} | 27299 | CTTTCTATATTGTTATC |
| | C1 | | decysin 1 | | down | | AGTCCAGG |
| | | | | | | | (SEQ ID NO: 5659) |
| | | | | | | | GACATTGGCTCTTTGTT |
| | | | | | | | TAGGCCTA |
| | | | | | | | (SEQ ID NO: 5660) |
| | | | | | | | GAATTTGTGACTTAGTT |
| | | | | | | | CTGCCCTT |
| | | | | | | | (SEQ ID NO: 5661) |
| | | | | | | | GAAAGCAGTCTTCCATC |
| | | | | | | | AAATCACC |
| | | | | | | | (SEQ ID NO: 5662) |
| | | | | | | | AATGCACGGCTAAACTA |
| | | | | | | | TTCAGAGT |
| | | | | | | | (SEQ ID NO: 5663) |
| | | | | | | | AAGTACTATGCTTTAAT |
| | | | | | | | GCTTCTTT |
| | | | | | | | (SEQ ID NO: 5664) |
| | | | | | | | GCTTCTTTCATCTTACT |
| | | | | | | | AGTATGGC |
| | | | | | | | (SEQ ID NO: 5665) |
| | | | | | | | TATGTTATTCCTCTGTG |
| | | | | | | | TTCACTTC |
| | | | | | | | (SEQ ID NO: 5666) |
| | | | | | | | ACTTCGCCTTGCTCTTG |
| | | | | | | | AAAGTGCA |
| | | | | | | | (SEQ ID NO: 5667) |
| | | | | | | | GTATATCCTATACACAC |
| | | | | | | | ATCTCCTT |
| | | | | | | | (SEQ ID NO: 5668) |
| | | | | | | | TGAGAAGTCTACATTGC |
| | | | | | | | TTACATTT |
| | | | | | | | (SEQ ID NO: 5669) |
| 204386_s_at | MRP63 | MGC3243, | mitochondrial | chr13q12.11 | Gain_{—} | 78988 | GAAATGGTCCTAATCCT |
| | | bMRP63 | ribosomal | | up | | GAGTCGTC |
| | | | protein 63 | | | | (SEQ ID NO: 5670) |
| | | | | | | | TGAGTCGTCACCCTTGG |
| | | | | | | | ATTTTATG |
| | | | | | | | (SEQ ID NO: 5671) |
| | | | | | | | ATGGATCACGGAGCTGA |
| | | | | | | | CCATCTTT |
| | | | | | | | (SEQ ID NO: 5672) |
| | | | | | | | GACCATCTTTACCTGGT |
| | | | | | | | CCTGGAAC |
| | | | | | | | (SEQ ID NO: 5673) |
| | | | | | | | GAAAATGAGCCTTTGGA |
| | | | | | | | CCAGTCTT |
| | | | | | | | (SEQ ID NO: 5674) |
| | | | | | | | GAGCCTTTGGACCAGTC |
| | | | | | | | TTTATTAA |
| | | | | | | | (SEQ ID NO: 5675) |
| | | | | | | | TGAGTAGTCTGCATATC |
| | | | | | | | GAATATCT |
| | | | | | | | (SEQ ID NO: 5676) |
| | | | | | | | GAATATCTAGAGCTCTA |
| | | | | | | | AACCCCCC |
| | | | | | | | (SEQ ID NO: 5677) |
| | | | | | | | GTCCTGTGGTTTCATTA |
| | | | | | | | GTCTGATA |
| | | | | | | | (SEQ ID NO: 5678) |
| | | | | | | | AGGGATTTCCTCAGTCA |
| | | | | | | | CAGATGAT |
| | | | | | | | (SEQ ID NO: 5679) |
| | | | | | | | GGAAAGCTGCAATAAAG |
| | | | | | | | CCACAATG |
| | | | | | | | (SEQ ID NO: 5680) |
| 209368_at | EPHX2 | CEH, SEH | epoxide | chr8p21-p12 | Loss_{—} | 2053 | GAACAGAACCTGAGTCG |
| | | | hydrolase 2, | | down | | TCGGACTT |
| | | | cytoplasmic | | | | (SEQ ID NO: 5681) |
| | | | | | | | GGACTTTCAAAAGCCTC |
| | | | | | | | TTCAGAGC |
| | | | | | | | (SEQ ID NO: 5682) |
| | | | | | | | GAGGAAATCCAGTTCT |
| | | | | | | | ATGTGCAGC |
| | | | | | | | (SEQ ID NO: 5683) |
| | | | | | | | CAGAGGTCCTCTAAACT |
| | | | | | | | GGTACCGA |
| | | | | | | | (SEQ ID NO: 5684) |
| | | | | | | | GGACGGAAGATCCTGAT |
| | | | | | | | TCCGGCCC |
| | | | | | | | (SEQ ID NO: 5685) |
| | | | | | | | GCCCTGATGGTCACGGC |
| | | | | | | | GGAGAAGG |
| | | | | | | | (SEQ ID NO: 5686) |
| | | | | | | | CGGAGAAGGACTTCGTG |
| | | | | | | | CTCGTTCC |
| | | | | | | | (SEQ ID NO: 5687) |
| | | | | | | | GCACATGGAGGACTGGA |
| | | | | | | | TTCCCCAC |
| | | | | | | | (SEQ ID NO: 5688) |
| | | | | | | | TGGCTGGATTCTGATGC |
| | | | | | | | CCGGAACC |
| | | | | | | | (SEQ ID NO: 5689) |
| | | | | | | | GGAACCCACCGGTGGTC |
| | | | | | | | TCAAAGAT |
| | | | | | | | (SEQ ID NO: 5690) |
| | | | | | | | GTAGAACGCAGCGTGTG |
| | | | | | | | CCCACGCT |
| | | | | | | | (SEQ ID NO: 5691) |
| 206790_s_at | NDUFB1 | CI-SGDH, | NADH | chr14q32.12 | Loss_{—} | 4707 | GAACTTACTTCAGATTG |
| | | MNLL | dehydrogenase | | down | | TGCGGGAC |
| | | | (ubiquinone) 1 | | | | (SEQ ID NO: 5692) |
| | | | beta | | | | CAGATTGTGCGGGACCA |
| | | | subcomplex, | | | | CTGGGTTC |
| | | | 1, 7 kDa | | | | (SEQ ID NO: 5693) |
| | | | | | | | GGACCACTGGGTTCATG |
| | | | | | | | TTCTTGTC |
| | | | | | | | (SEQ ID NO: 5694) |
| | | | | | | | CTGGGTTCATGTTCTTG |
| | | | | | | | TCCCTATG |
| | | | | | | | (SEQ ID NO: 5695) |
| | | | | | | | TGTCCCTATGGGATTTG |
| | | | | | | | TCATTGGA |
| | | | | | | | (SEQ ID NO: 5696) |
| | | | | | | | GTGATGAACGGCTAACT |
| | | | | | | | GCCTTCCG |
| | | | | | | | (SEQ ID NO: 5697) |
| | | | | | | | GCTAACTGCCTTCCGGA |
| | | | | | | | ACAAGAGT |
| | | | | | | | (SEQ ID NO: 5698) |
| | | | | | | | ACTGCCTTCCGGAACAA |
| | | | | | | | GAGTATGT |
| | | | | | | | (SEQ ID NO: 5699) |
| | | | | | | | AAAGGGAATTGCAACCC |
| | | | | | | | AGTGAAGA |
| | | | | | | | (SEQ ID NO: 5700) |
| | | | | | | | GACTGGCTAGATTATCG |
| | | | | | | | AATGTTCA |
| | | | | | | | (SEQ ID NO: 5701) |
| | | | | | | | GTTCACATTTTAAAGTT |
| | | | | | | | CTGAGAGA |
| | | | | | | | (SEQ ID NO: 5702) |
| 204149_s_at | GSTM4 | GSTM4-4, | glutathione | chr1p13.3 | Loss_{—} | 2948 | GAGAACCAGGCTATGGA |
| | | GTM4, | S-transferase | | down | | CGTCTCCA |
| | | MGC131945, | M4 | | | | (SEQ ID NO: 5703) |
| | | MGC9247 | | | | | CAGGCTATGGACGTCTC |
| | | | | | | | CAATCAGC |
| | | | | | | | (SEQ ID NO: 5704) |
| | | | | | | | CTATGGACGTCTCCAAT |
| | | | | | | | CAGCTGGC |
| | | | | | | | (SEQ ID NO: 5705) |
| | | | | | | | CTCCAATCAGCTGGCCA |
| | | | | | | | GAGTCTGC |
| | | | | | | | (SEQ ID NO: 5706) |
| | | | | | | | TCTGCTACAGCCCTGAC |
| | | | | | | | TTTGAGAA |
| | | | | | | | (SEQ ID NO: 5707) |
| | | | | | | | GCTACAGCCCTGACTTT |
| | | | | | | | GAGAAACT |
| | | | | | | | (SEQ ID NO: 5708) |
| | | | | | | | ATACTTGGAGGAACTTC |
| | | | | | | | CTACAATG |
| | | | | | | | (SEQ ID NO: 5709) |
| | | | | | | | GAGGAACTTCCTACAAT |
| | | | | | | | GATGCAGC |
| | | | | | | | (SEQ ID NO: 5710) |
| | | | | | | | GGAACTTCCTACAATGA |
| | | | | | | | TGCAGCAC |
| | | | | | | | (SEQ ID NO: 5711) |
| | | | | | | | ACTTCCTACAATGATGC |
| | | | | | | | AGCACTTC |
| | | | | | | | (SEQ ID NO: 5712) |
| | | | | | | | GAAGAGGCCATGGTTTG |
| | | | | | | | TTGGAGAC |
| | | | | | | | (SEQ ID NO: 5713) |
| 208909_at | UQCRFS1 | RIP1, | ubiquinol- | chr19q12- | Loss_{—} | 7386 | GAGAGGCAAACCCCTGT |
| | | RIS1, | cytochrome c | q13.1 | down | | TTGTGCGT |
| | | RISP, | reductase, | | | | (SEQ ID NO: 5714) |
| | | UQCR5 | Rieske | | | | CTGTTTGTGCGTCATAG |
| | | | iron-sulfur | | | | AACCCAGA |
| | | | polypeptide 1 | | | | (SEQ ID NO: 5715) |
| | | | | | | | GGACCCACAGCATGATC |
| | | | | | | | TAGATCGA |
| | | | | | | | (SEQ ID NO: 5716) |
| | | | | | | | GATAGGTGTTTGCACTC |
| | | | | | | | ATCTTGGC |
| | | | | | | | (SEQ ID NO: 5717) |
| | | | | | | | TCATCTTGGCTGTGTAC |
| | | | | | | | CCATTGCA |
| | | | | | | | (SEQ ID NO: 5718) |
| | | | | | | | TTTTGGTGGTTATTACT |
| | | | | | | | GCCCTTGC |
| | | | | | | | (SEQ ID NO: 5719) |
| | | | | | | | GGGTCACACTATGATGC |
| | | | | | | | ATCTGGCA |
| | | | | | | | (SEQ ID NO: 5720) |
| | | | | | | | CAGGATCAGATTGGGTC |
| | | | | | | | CTGCTCCT |
| | | | | | | | (SEQ ID NO: 5721) |
| | | | | | | | TGAAGTCCCCACGTATG |
| | | | | | | | AGTTCACC |
| | | | | | | | (SEQ ID NO: 5722) |
| | | | | | | | GGACTCAAGTCATAGGC |
| | | | | | | | TTCTTTCA |
| | | | | | | | (SEQ ID NO: 5723) |
| | | | | | | | GGCTTCTTTCAGTCTTT |
| | | | | | | | ATGTCACC |
| | | | | | | | (SEQ ID NO: 5724) |
| 221432_s_at | SLC25A | MFRN2; | solute carrier | chr10q23- | Loss_{—} | 81894 | GCAGATGTACAACTCAC |
| | 28 | MRS4L; | family 25, | q24 | down | | CATACCAC |
| | | MRS3/4; | member 28 | | | | (SEQ ID NO: 5725) |
| | | NPD016; | | | | | AATTCCTGCAGGAGCAC |
| | | DKFZp547C | | | | | TTTAACCC |
| | | 109 | | | | | (SEQ ID NO: 5726) |
| | | | | | | | CCAGAGACGGTACAACC |
| | | | | | | | CAAGCTCC |
| | | | | | | | (SEQ ID NO: 5727) |
| | | | | | | | TTGCGCAGGAGCTGTA |
| | | | | | | | GCTGCCGCA |
| | | | | | | | (SEQ ID NO: 5728) |
| | | | | | | | CACAACCCCACTGGACG |
| | | | | | | | TTTGCAAA |
| | | | | | | | (SEQ ID NO: 5729) |
| | | | | | | | TTGCAAAACACTGCTCA |
| | | | | | | | ACACCCAG |
| | | | | | | | (SEQ ID NO: 5730) |
| | | | | | | | CACCCAGGAGTCCTTGG |
| | | | | | | | CTTTGAAC |
| | | | | | | | (SEQ ID NO: 5731) |
| | | | | | | | GCATGGCTAGTGCCTTC |
| | | | | | | | AGGACGGT |
| | | | | | | | (SEQ ID NO: 5732) |
| | | | | | | | CAAGTAGGTGGGGTGAC |
| | | | | | | | CGCCTATT |
| | | | | | | | (SEQ ID NO: 5733) |
| | | | | | | | ATTTCCGAGGGGTGCAG |
| | | | | | | | GCCAGAGT |
| | | | | | | | (SEQ ID NO: 5734) |
| | | | | | | | AGCCATCGCATGGTCTG |
| | | | | | | | TGTATGAG |
| | | | | | | | (SEQ ID NO: 5735) |
| 211755_s_at | ATP5F1 | MGC24431, | ATP synthase, | chr1p13.2 | Loss_{—} | 515 | GCCTGGACTATCATATA |
| | | PIG47 | H+ transporting, | | down | | TCTGTGCA |
| | | | mitochondrial F0 | | | | (SEQ ID NO: 5736) |
| | | | complex, subunit | | | | ATCTGTGCAGAACATGA |
| | | | B1 | | | | TGCGTCGA |
| | | | | | | | (SEQ ID NO: 5737) |
| | | | | | | | AAGCACGTGGTGCAAAG |
| | | | | | | | CATCTCCA |
| | | | | | | | (SEQ ID NO: 5738) |
| | | | | | | | GACAATTGCCAAGTGCA |
| | | | | | | | TTGCGGAC |
| | | | | | | | (SEQ ID NO: 5739) |
| | | | | | | | GCATTGCGGACCTAAAG |
| | | | | | | | CTGCTGGC |
| | | | | | | | (SEQ ID NO: 5740) |
| | | | | | | | GAAGGCTCAAGCACAGC |
| | | | | | | | CAGTTATG |
| | | | | | | | (SEQ ID NO: 5741) |
| | | | | | | | TGACAAAGTCTTTCTGT |
| | | | | | | | GTTGGTGT |
| | | | | | | | (SEQ ID NO: 5742) |
| | | | | | | | GAACGAAATCTCTATCG |
| | | | | | | | GCCAGTCA |
| | | | | | | | (SEQ ID NO: 5743) |
| | | | | | | | ATCGGCCAGTCAGATGT |
| | | | | | | | TTCTCATC |
| | | | | | | | (SEQ ID NO: 5744) |
| | | | | | | | AAACTTGCTGCCTGACT |
| | | | | | | | AAAGATTA |
| | | | | | | | (SEQ ID NO: 5745) |
| | | | | | | | AATTAATTCTACCATCT |
| | | | | | | | TGCAATAA |
| | | | | | | | (SEQ ID NO: 5746) |
| 214937_x_at | PCM1 | CXXC3, | pericentriolar | chr8p22- | Loss_{—} | 5108 | GGAAGTCCTGATACTGA |
| | | PCM1, RFT | material 1 | p21.3 | down | | ATCTCCAG |
| | | | | | | | (SEQ ID NO: 5747) |
| | | | | | | | GAATCTCCAGTGTTAGT |
| | | | | | | | GAATGACT |
| | | | | | | | (SEQ ID NO: 5748) |
| | | | | | | | GAAGATTTACCACTGAA |
| | | | | | | | ACTGACAA |
| | | | | | | | (SEQ ID NO: 5749) |
| | | | | | | | GAACCTGAAACGGTGGG |
| | | | | | | | AGCCCAGA |
| | | | | | | | (SEQ ID NO: 5750) |
| | | | | | | | GGGAGCCCAGAGTATAT |
| | | | | | | | GAGATGTC |
| | | | | | | | (SEQ ID NO: 5751) |
| | | | | | | | ATGTCTTCAGAGGCTCA |
| | | | | | | | TCTAACTC |
| | | | | | | | (SEQ ID NO: 5752) |
| | | | | | | | CATCTAACTCTGTCCTT |
| | | | | | | | ACATACTC |
| | | | | | | | (SEQ ID NO: 5753) |
| | | | | | | | GTTTGACACTGCTTTTT |
| | | | | | | | TGATAGGT |
| | | | | | | | (SEQ ID NO: 5754) |
| | | | | | | | TGATAGGTGTGGTCATT |
| | | | | | | | TCTCCCCA |
| | | | | | | | (SEQ ID NO: 5755) |
| | | | | | | | GATTTAAGCCTTGACAC |
| | | | | | | | ACTGTGTT |
| | | | | | | | (SEQ ID NO: 5756) |
| | | | | | | | ATTTTTTTCCCCATTGT |
| | | | | | | | GATGTTTG |
| | | | | | | | (SEQ ID NO: 5757) |
| 206441_s_at | COMMD4 | FLJ20452 | COMM domain | chr15q24.2 | Loss_{—} | 54939 | GGAGCTGCTGGGACAGG |
| | | | containing 4 | | down | | GGATTGAT |
| | | | | | | | (SEQ ID NO: 5758) |
| | | | | | | | AGCTCACGGCTGACGCC |
| | | | | | | | AAGTTTGA |
| | | | | | | | (SEQ ID NO: 5759) |
| | | | | | | | TCCAGTGCGGCCAAGCA |
| | | | | | | | CAGTGTCG |
| | | | | | | | (SEQ ID NO: 5760) |
| | | | | | | | AGTGTCGATGGCGAATC |
| | | | | | | | CTTGTCCA |
| | | | | | | | (SEQ ID NO: 5761) |
| | | | | | | | TCCTTGTCCAGTGAACT |
| | | | | | | | GCAGCAGC |
| | | | | | | | (SEQ ID NO: 5762) |
| | | | | | | | AGCTGGGGCTGCCCAAA |
| | | | | | | | GAGCACGC |
| | | | | | | | (SEQ ID NO: 5763) |
| | | | | | | | CCAGCCTGTGCCGCTGT |
| | | | | | | | TATGAGGA |
| | | | | | | | (SEQ ID NO: 5764) |
| | | | | | | | CAAAGCCCCTTGCAGAA |
| | | | | | | | GCACTTGC |
| | | | | | | | (SEQ ID NO: 5765) |
| | | | | | | | TGAACTGCTCTTCGGGA |
| | | | | | | | GGCAGCCC |
| | | | | | | | (SEQ ID NO: 5766) |
| | | | | | | | GCCCTGGTTCTAGGATG |
| | | | | | | | CTGAGGCC |
| | | | | | | | (SEQ ID NO: 5767) |
| | | | | | | | GCCTCACTTCTCTCTTG |
| | | | | | | | AGAACTTG |
| | | | | | | | (SEQ ID NO: 5768) |
| 219303_at | C13 | RNF219; | ring finger | chr13q31.1 | Gain_{—} | 79596 | GGCATGGAGCACTTGGG |
| | orf7 | FLJ13449; | protein 219 | | up | | TTTAGAGG |
| | | FLJ25774; | | | | | (SEQ ID NO: 5769) |
| | | DKFZp686A | | | | | AAAACATCATAGCTTCA |
| | | 01276; | | | | | TTGTTCCA |
| | | DKFZp686N | | | | | (SEQ ID NO: 5770) |
| | | 15250; | | | | | GCTTCATTGTTCCAGAT |
| | | DKFZp686O | | | | | GTAACAGG |
| | | 03173 | | | | | (SEQ ID NO: 5771) |
| | | | | | | | CATTCCAGGGGAGTTTT |
| | | | | | | | CTTTTGAG |
| | | | | | | | (SEQ ID NO: 5772) |
| | | | | | | | TTTGCATGTTCCTGTTC |
| | | | | | | | TTTGTGGA |
| | | | | | | | (SEQ ID NO: 5773) |
| | | | | | | | TCCTGTTCTTTGTGGAA |
| | | | | | | | ACTATGCA |
| | | | | | | | (SEQ ID NO: 5774) |
| | | | | | | | TTTTGCTTGCTGTGTTT |
| | | | | | | | TCCATACT |
| | | | | | | | (SEQ ID NO: 5775) |
| | | | | | | | ATGTAGGACAAAACTTTT |
| | | | | | | | CTGTGAA |
| | | | | | | | (SEQ ID NO: 5776) |
| | | | | | | | CGTATTTGGTATGCCTA |
| | | | | | | | TATAGGTC |
| | | | | | | | (SEQ ID NO: 5777) |
| | | | | | | | AATGGGTTTGTATGCTG |
| | | | | | | | TTTAATGT |
| | | | | | | | (SEQ ID NO: 5778) |
| | | | | | | | GTATGCTGTTTAATGTG |
| | | | | | | | CACTGAAC |
| | | | | | | | (SEQ ID NO: 5779) |
| 210107_at | CLCA1 | CACC, | CLCA family | chr1p31-p22 | Loss_{—} | 1179 | GGCCAAATCACCGACCT |
| | | CACC1, | member 1, | | down | | GAAGGCGG |
| | | CLCRG1, | chloride | | | | (SEQ ID NO: 5780) |
| | | FLJ95147, | channel | | | | TTCACGGGGGCAGTCT |
| | | GOB5 | regulator | | | | CATTAATCT |
| | | | | | | | (SEQ ID NO: 5781) |
| | | | | | | | GACTTGGACAGCTCCTG |
| | | | | | | | GGGATGAT |
| | | | | | | | (SEQ ID NO: 5782) |
| | | | | | | | CAAGTGAATACTACTGC |
| | | | | | | | TCTCATCC |
| | | | | | | | (SEQ ID NO: 5783) |
| | | | | | | | GGCACAGATCTTTTCA |
| | | | | | | | TTGCTATTC |
| | | | | | | | (SEQ ID NO: 5784) |
| | | | | | | | ATCTTTGTTTATTCCTC |
| | | | | | | | CACAGACT |
| | | | | | | | (SEQ ID NO: 5785) |
| | | | | | | | CGCCAGAGACACCTAGT |
| | | | | | | | CCTGATGA |
| | | | | | | | (SEQ ID NO: 5786) |
| | | | | | | | TAGTCCTGATGAAACGT |
| | | | | | | | CTGCTCCT |
| | | | | | | | (SEQ ID NO: 5787) |
| | | | | | | | TCTGCTCCTTGTCCTAA |
| | | | | | | | TATTCATA |
| | | | | | | | (SEQ ID NO: 5788) |
| | | | | | | | TATCAACAGCACCATTC |
| | | | | | | | CTGGCATT |
| | | | | | | | (SEQ ID NO: 5789) |
| | | | | | | | ACTGCAGCTGTCAATAG |
| | | | | | | | CCTAGGGC |
| | | | | | | | (SEQ ID NO: 5790) |
| 218801_at | UGCGL2 | RP11- | UDP-glucose | chr13q32.1 | Gain_{—} | 55757 | GGCTCAGGAGCCAGTAT |
| | | 31K22.2, | ceramide | | up | | CAAGCTCT |
| | | FLJ10873, | glucosyl- | | | | (SEQ ID NO: 5791) |
| | | FLJ11485, | transferase- | | | | AAGCTCTCAGTCAAGA |
| | | HUGT2, | like 2 | | | | TCCAAACAG |
| | | MGC117360, | | | | | (SEQ ID NO: 5792) |
| | | MGC150689, | | | | | GATCCAAACAGTCTTTC |
| | | MGC87276 | | | | | AAACCTAG |
| | | | | | | | (SEQ ID NO: 5793) |
| | | | | | | | AGATCAGGATCTCCCCA |
| | | | | | | | ATAATATG |
| | | | | | | | (SEQ ID NO: 5794) |
| | | | | | | | TACCAAGTCGCCATTAA |
| | | | | | | | GTCTCTTC |
| | | | | | | | (SEQ ID NO: 5795) |
| | | | | | | | TCTTCCTCAAGACTGGC |
| | | | | | | | TGTGGTGT |
| | | | | | | | (SEQ ID NO: 5796) |
| | | | | | | | GAATCCAAACTAAAAGC |
| | | | | | | | TGCTGCCA |
| | | | | | | | (SEQ ID NO: 5797) |
| | | | | | | | CTGCCAGAATTGTCCCA |
| | | | | | | | GAATGGGT |
| | | | | | | | (SEQ ID NO: 5798) |
| | | | | | | | GACACATGATGAACTCT |
| | | | | | | | AGCACTGG |
| | | | | | | | (SEQ ID NO: 5799) |
| | | | | | | | TGCTGGGGAAGTCTGGA |
| | | | | | | | GCCCCTGC |
| | | | | | | | (SEQ ID NO: 5800) |
| | | | | | | | AGCCCCTGCTGAGACGA |
| | | | | | | | TTTGGAAG |
| | | | | | | | (SEQ ID NO: 5801) |
| 203478_at | NDUFC1 | KFYI; | NADH | chr4q28.2- | Loss_{—} | 4717 | GGGGCCCAAGGAATTCG |
| | | MGC117464; | dehydrogenase | q31.1 | down | | CAAGATGG |
| | | MGC126847; | (ubiquinone) 1, | | | | (SEQ ID NO: 5802) |
| | | MGC138266 | subcomplex | | | | AGGAATTCGCAAGATG |
| | | | unknown, 1, | | | | GCGCCGTCC |
| | | | 6 kDa | | | | (SEQ ID NO: 5803) |
| | | | | | | | GCCCTTCAGTGCGATCA |
| | | | | | | | AAGTTCTA |
| | | | | | | | (SEQ ID NO: 5804) |
| | | | | | | | GTGCGATCAAAGTTCTA |
| | | | | | | | CGTGCGAG |
| | | | | | | | (SEQ ID NO: 5805) |
| | | | | | | | TTCTACGTGCGAGAGCC |
| | | | | | | | GCCGAATG |
| | | | | | | | (SEQ ID NO: 5806) |
| | | | | | | | CCGCCGAATGCCAAACC |
| | | | | | | | TGACTGGC |
| | | | | | | | (SEQ ID NO: 5807) |
| | | | | | | | ACCTGACTGGCTGAAAG |
| | | | | | | | TTGGGTTC |
| | | | | | | | (SEQ ID NO: 5808) |
| | | | | | | | TGAAAGTTGGGTTCACC |
| | | | | | | | TTGGGCAC |
| | | | | | | | (SEQ ID NO: 5809) |
| | | | | | | | GTCTTCTTGTGGATCTA |
| | | | | | | | TCTCATCA |
| | | | | | | | (SEQ ID NO: 5810) |
| | | | | | | | GGATCTATCTCATCAAA |
| | | | | | | | CAACACAA |
| | | | | | | | (SEQ ID NO: 5811) |
| | | | | | | | CAAGCCAGCATTTGTAT |
| | | | | | | | TTTGCATC |
| | | | | | | | (SEQ ID NO: 5812) |
| 218738_s_at | RNF138 | HSD-4, | ring finger | chr18q12.1 | Loss_{—} | 51444 | GGGGGTTGAAGTGTTTA |
| | | MGC8758, | protein 138 | | down | | TCTGATTT |
| | | NARF, | | | | | (SEQ ID NO: 5813) |
| | | STRIN | | | | | AGTGCTAAGTTATCTAG |
| | | | | | | | TTGGCTAC |
| | | | | | | | (SEQ ID NO: 5814) |
| | | | | | | | TAGTTGGCTACTATTAC |
| | | | | | | | ACCTTAAA |
| | | | | | | | (SEQ ID NO: 5815) |
| | | | | | | | ACAATTACCTGTTTATA |
| | | | | | | | TGGGCTC |
| | | | | | | | (SEQ ID NO: 5816) |
| | | | | | | | TATGGTGCTCATTTGTT |
| | | | | | | | ATTCTCAA |
| | | | | | | | (SEQ ID NO: 5817) |
| | | | | | | | GAGAAAGATTCTACCAA |
| | | | | | | | CCACTGTT |
| | | | | | | | (SEQ ID NO: 5818) |
| | | | | | | | TACCAACCACTGTTCAC |
| | | | | | | | TACTTT |
| | | | | | | | (SEQ ID NO: 5819) |
| | | | | | | | GTAGAAGGCCCAAATCA |
| | | | | | | | CAGAATAA |
| | | | | | | | (SEQ ID NO: 5820) |
| | | | | | | | GGATTTGCTGACATTCC |
| | | | | | | | ATACTAAT |
| | | | | | | | (SEQ ID NO: 5821) |
| | | | | | | | GAATCTCAGAAGTAGTT |
| | | | | | | | TGCTGCTA |
| | | | | | | | (SEQ ID NO: 5822) |
| | | | | | | | AACCCTTGTTGACTTTT |
| | | | | | | | CTACACTG |
| | | | | | | | (SEQ ID NO: 5823) |
| 202956_at | ARFGEF | ARFGEP1, | ADP-ribosylation | chr8q13 | Gain_{—} | 10565 | GGTAGGTGGTGATTTTG |
| | 1 | BIG1, | factor guanine | | up | | AGGCTGTA |
| | | D730028O1 | nucleotide- | | | | (SEQ ID NO: 5824) |
| | | 8Rik, | exchange factor | | | | TGAGGCTGTAACATGCC |
| | | DKFZP434L | 1(brefeldin A- | | | | CAGAAGCT |
| | | 057, P200 | inhibited) | | | | (SEQ ID NO: 5825) |
| | | | | | | | GAAGCTGTTGTGGCCGA |
| | | | | | | | CACTTCAA |
| | | | | | | | (SEQ ID NO: 5826) |
| | | | | | | | GTGGCCGACACTTCAAC |
| | | | | | | | AATAGGGA |
| | | | | | | | (SEQ ID NO: 5827) |
| | | | | | | | ATATCCCTACTGACAGT |
| | | | | | | | AACTACCT |
| | | | | | | | (SEQ ID NO: 5828) |
| | | | | | | | GTAACTACCTGTCACAT |
| | | | | | | | ATTTCTCT |
| | | | | | | | (SEQ ID NO: 5829) |
| | | | | | | | CTTTTGGGTGGTGGGGC |
| | | | | | | | TTGATGTA |
| | | | | | | | (SEQ ID NO: 5830) |
| | | | | | | | GGCATGGTTTGCGGAGG |
| | | | | | | | TTAGATTT |
| | | | | | | | (SEQ ID NO: 5831) |
| | | | | | | | GTGAATTGTGCTCTGAT |
| | | | | | | | GGTTAAAA |
| | | | | | | | (SEQ ID NO: 5832) |
| | | | | | | | AGATTGTCAAGCATTCC |
| | | | | | | | GTATTAAC |
| | | | | | | | (SEQ ID NO: 5833) |
| | | | | | | | ATTGATTCCCATCTGGC |
| | | | | | | | ATATTCTA |
| | | | | | | | (SEQ ID NO: 5834) |
| 208884_s_at | LOC | LOC730429 | ubiquitin | chr8q22 | Gain_{—} | 51366 | GGTAGTTGCAGAACAGC |
| | 730429 | | protein | | up | | CCTTACAT |
| | | | ligase E3 | | | | (SEQ ID NO: 5835) |
| | | | component | | | | AGGTCTACTAGATGTGC |
| | | | n-recognin 5 | | | | TTCCAAAA |
| | | | | | | | (SEQ ID NO: 5836) |
| | | | | | | | AAATGCTGATCAGTTTT |
| | | | | | | | ACCTCTTT |
| | | | | | | | (SEQ ID NO: 5837) |
| | | | | | | | GCAGTTCAAGCGTTGGT |
| | | | | | | | TCTGGTCA |
| | | | | | | | (SEQ ID NO: 5838) |
| | | | | | | | TTTTGGACATCAAGCCC |
| | | | | | | | ATCACTGC |
| | | | | | | | (SEQ ID NO: 5839) |
| | | | | | | | GAAGAAGGATTCCAGCC |
| | | | | | | | TATGCCCT |
| | | | | | | | (SEQ ID NO: 5840) |
| | | | | | | | GCCTATGCCCTCAATCA |
| | | | | | | | CAATAAGA |
| | | | | | | | (SEQ ID NO: 5841) |
| | | | | | | | CCAACATCTTCCTACTG |
| | | | | | | | CAAATACT |
| | | | | | | | (SEQ ID NO: 5842) |
| | | | | | | | ATACTTGCATTTCTCGA |
| | | | | | | | CTTTACGT |
| | | | | | | | (SEQ ID NO: 5843) |
| | | | | | | | TCCCACTCTATTCCTCT |
| | | | | | | | AAACAGAT |
| | | | | | | | (SEQ ID NO: 5844) |
| | | | | | | | AATTGTTACTCGCCATT |
| | | | | | | | AAGACCAA |
| | | | | | | | (SEQ ID NO: 5845) |
| 203596_s_at | IFIT5 | RI58; | interferon- | chr10q23.31 | Loss_{—} | 24138 | GTACATAACTTCTATCA |
| | | FLJ53857; | induced | | down | | CTTGCCAC |
| | | FLJ92678 | protein with | | | | (SEQ ID NO: 5846) |
| | | | tetratricopep- | | | | AAATCAGGGCCTGTGTT |
| | | | tide repeats 5 | | | | CATACACA |
| | | | | | | | (SEQ ID NO: 5847) |
| | | | | | | | GGCCTGAGATTTCTGCA |
| | | | | | | | CTTTAAAC |
| | | | | | | | (SEQ ID NO: 5848) |
| | | | | | | | GTAAGGTCTACAGCATT |
| | | | | | | | GTTCCTCA |
| | | | | | | | (SEQ ID NO: 5849) |
| | | | | | | | ATTGTTCCTCAAACTTG |
| | | | | | | | GCTACGTA |
| | | | | | | | (SEQ ID NO: 5850) |
| | | | | | | | TTGGCTACGTATTGGAA |
| | | | | | | | TCACCTAA |
| | | | | | | | (SEQ ID NO: 5851) |
| | | | | | | | CAAAACATGGATGTCTG |
| | | | | | | | GGTCCCGC |
| | | | | | | | (SEQ ID NO: 5852) |
| | | | | | | | GTCCCGCCCCATAGAGA |
| | | | | | | | ATGACTTA |
| | | | | | | | (SEQ ID NO: 5853) |
| | | | | | | | AATTGGCATGGGGTGCA |
| | | | | | | | GTCCAGGC |
| | | | | | | | (SEQ ID NO: 5854) |
| | | | | | | | GTTTGCATTTGGGACTC |
| | | | | | | | CTCATCAT |
| | | | | | | | (SEQ ID NO: 5855) |
| | | | | | | | GGACTCCTCATCATATA |
| | | | | | | | CTTGGTAT |
| | | | | | | | (SEQ ID NO: 5856) |
| 212592_at | IGJ | IGCJ, JCH | immunoglobulin J | chr4q21 | Loss_{—} | 3512 | GTATCAAAATCTTCCAA |
| | | | polypeptide, | | down | | TTATCATG |
| | | | linker protein | | | | (SEQ ID NO: 5857) |
| | | | for immuno- | | | | ATCTTCCAATTATCAT |
| | | | globulin | | | | GCTCACCTG |
| | | | alpha and mu | | | | (SEQ ID NO: 5858) |
| | | | polypeptides | | | | TCCAATTATCATGCTCA |
| | | | | | | | CCTGAAAG |
| | | | | | | | (SEQ ID NO: 5859) |
| | | | | | | | GCTCACCTGAAAGAGGT |
| | | | | | | | ATGCTCTC |
| | | | | | | | (SEQ ID NO: 5860) |
| | | | | | | | GAGGTATGCTCTCTTAG |
| | | | | | | | GAATACAG |
| | | | | | | | (SEQ ID NO: 5861) |
| | | | | | | | TAGGAATACAGTTTCTA |
| | | | | | | | GCATTAAA |
| | | | | | | | (SEQ ID NO: 5862) |
| | | | | | | | TAATAAAATGTTCCTCG |
| | | | | | | | CATTCCCC |
| | | | | | | | (SEQ ID NO: 5863) |
| | | | | | | | AGTTTCACTGTGTAGAG |
| | | | | | | | AACATATA |
| | | | | | | | (SEQ ID NO: 5864) |
| | | | | | | | ATAGGTCAATTATATGT |
| | | | | | | | CTCCATTA |
| | | | | | | | (SEQ ID NO: 5865) |
| | | | | | | | AAACATGTTCTAGAACT |
| | | | | | | | AGTTACAA |
| | | | | | | | (SEQ ID NO: 5866) |
| | | | | | | | AAATATATTTGGACATA |
| | | | | | | | ACAGACTT |
| | | | | | | | (SEQ ID NO: 5867) |
| 212692_s_at | LRBA | BGL, | LPS-responsive | chr4q31.3 | Loss_{—} | 987 | GTCTGTCTATCAATATT |
| | | CDC4L, | vesicle | | down | | GTTCTTCA |
| | | DKFZp686A | trafficking, | | | | (SEQ ID NO: 5868) |
| | | 09128, | beach and | | | | TTTCCTGATCTGTCATC |
| | | DKFZp686K | anchor | | | | TTCATAGC |
| | | 03100, | containing | | | | (SEQ ID NO: 5869) |
| | | DKFZp686P | | | | | AATGCTCTTGAGCTCA |
| | | 2258, | | | | | CAACATTTG |
| | | FLJ16600, | | | | | (SEQ ID NO: 5870) |
| | | FLJ25686, | | | | | ACCAAAGCTCACTACTG |
| | | LAB300, | | | | | CGGTTTGC |
| | | LBA, | | | | | (SEQ ID NO: 5871) |
| | | MGC72098 | | | | | GTTTGCCTGTGCCTGGA |
| | | | | | | | CAATGAGG |
| | | | | | | | (SEQ ID NO: 5872) |
| | | | | | | | GGACAATGAGGCGGAGC |
| | | | | | | | CACTGTTG |
| | | | | | | | (SEQ ID NO: 5873) |
| | | | | | | | TCCCCGGGTTTGCAAAT |
| | | | | | | | AGAGGCTA |
| | | | | | | | (SEQ ID NO: 5874) |
| | | | | | | | TAGAGGCTACCGGGTGC |
| | | | | | | | TGTATTCA |
| | | | | | | | (SEQ ID NO: 5875) |
| | | | | | | | GTTATTAAACTATCATC |
| | | | | | | | TCCACCTT |
| | | | | | | | (SEQ ID NO: 5876) |
| | | | | | | | CTCCACCTTCCTTTTGA |
| | | | | | | | TTAGCAAT |
| | | | | | | | (SEQ ID NO: 5877) |
| | | | | | | | GGTGGTGTATAATTCTA |
| | | | | | | | CTTTTCTA |
| | | | | | | | (SEQ ID NO: 5878) |
| 202548_s_at | ARHGEF | RP11- | Rho guanine | chr13q34 | Gain_{—} | 8874 | GTTACGGCATTGCCTTT |
| | 7 | 494P5.1, | nucleotide | | up | | TCTTTCTG |
| | | BETA-PIX, | exchange | | | | (SEQ ID NO: 5879) |
| | | COOL1, | factor | | | | TTCTGTGGATCCAGTAT |
| | | DKFZp686C | (GEF) 7 | | | | CTTCCTCG |
| | | 12170, | | | | | (SEQ ID NO: 5880) |
| | | DKFZp761K | | | | | ATCTTCCTCGGCTTTTT |
| | | 1021, | | | | | AGGGAGCA |
| | | KIAA0142, | | | | | (SEQ ID NO: 5881) |
| | | KIAA0412, | | | | | AAACCTGCCCTGTTG |
| | | Nbla10314, | | | | | TATATAACTG |
| | | P50, | | | | | (SEQ ID NO: 5882) |
| | | P50BP, | | | | | AACTGTGTCTGTTTCA |
| | | P85, | | | | | CCGTGTGAC |
| | | P85COOL1, | | | | | (SEQ ID NO: 5883) |
| | | P85SPR, | | | | | GCCACGATTTGCCCGAG |
| | | PAK3, PIXB | | | | | GGTTACTC |
| | | | | | | | (SEQ ID NO: 5884) |
| | | | | | | | CCTTTGCTCTCACCTT |
| | | | | | | | GTATGGATG |
| | | | | | | | (SEQ ID NO: 5885) |
| | | | | | | | GATGAAGCCATTTCTTA |
| | | | | | | | TCCTGTAG |
| | | | | | | | (SEQ ID NO: 5886) |
| | | | | | | | GTAGCATCAGAAGCTC |
| | | | | | | | GTTCCTTCA |
| | | | | | | | (SEQ ID NO: 5887) |
| | | | | | | | CACACTCAGTGGCGTCT |
| | | | | | | | GTGCTTGT |
| | | | | | | | (SEQ ID NO: 5888) |
| | | | | | | | CACCCAGGGCGCCTGTT |
| | | | | | | | GACAAGTG |
| | | | | | | | (SEQ ID NO: 5889) |
| 209627_s_at | OSBPL3 | DKFZp667P | oxysterol | chr7p15 | Gain_{—} | 26031 | GTTATGCCTTCTGGCTA |
| | | 1518, | binding | | up | | CTACAGAA |
| | | KIAA0704, | protein-like 3 | | | | (SEQ ID NO: 5890) |
| | | MGC21526, | | | | | ACAGAATCTCCGCAGAC |
| | | ORP-3, | | | | | CTTTTAGT |
| | | ORP3, | | | | | (SEQ ID NO: 5891) |
| | | OSBP3 | | | | | GAAGGTGTCCTTATGGT |
| | | | | | | | CTTAGGAA |
| | | | | | | | (SEQ ID NO: 5892) |
| | | | | | | | ATAGCTATTTTCATTT |
| | | | | | | | GCTCTATTT |
| | | | | | | | (SEQ ID NO: 5893) |
| | | | | | | | TGCTCTATTTTCCTCTG |
| | | | | | | | CATTGTAC |
| | | | | | | | (SEQ ID NO: 5894) |
| | | | | | | | GCATTGTACTAATCTGT |
| | | | | | | | TACTCAGT |
| | | | | | | | (SEQ ID NO: 5895) |
| | | | | | | | GTTACTCAGTGTTAGTC |
| | | | | | | | TTCATTTT |
| | | | | | | | (SEQ ID NO: 5896) |
| | | | | | | | GTGTTGGAAACCTGCGT |
| | | | | | | | CATGTAAA |
| | | | | | | | (SEQ ID NO: 5897) |
| | | | | | | | AATCTTCCACTTAACCA |
| | | | | | | | CTGAAAAT |
| | | | | | | | (SEQ ID NO: 5898) |
| | | | | | | | AGCTCCTATTAATTTGC |
| | | | | | | | TTATCCCC |
| | | | | | | | (SEQ ID NO: 5899) |
| | | | | | | | TATCCCCCTCATGTAGC |
| | | | | | | | TAGTTGAA |
| | | | | | | | (SEQ ID NO: 5900) |
| 212448_at | NEDD4L | FLJ33870, | neural precursor | chr18q21 | Loss_{—} | 23327 | GTTGTGCCTCTTGTGTG |
| | | KIAA0439, | cell expressed, | | down | | CTAGATTA |
| | | NEDD4-2, | developmentally | | | | (SEQ ID NO: 5901) |
| | | RSP5, | down-regulated | | | | ACTTGACTTGATCCTCT |
| | | hNedd4-2 | 4-like | | | | GAGCTCAA |
| | | | | | | | (SEQ ID NO: 5902) |
| | | | | | | | GGCAGAGGACTGAGGGT |
| | | | | | | | ACCTGCAC |
| | | | | | | | (SEQ ID NO: 5903) |
| | | | | | | | TGCACAGTTTGATTCTT |
| | | | | | | | TTCCACGT |
| | | | | | | | (SEQ ID NO: 5904) |
| | | | | | | | TTTTCCACGTGTAAGTC |
| | | | | | | | TCCATTGC |
| | | | | | | | (SEQ ID NO: 5905) |
| | | | | | | | GTCTCCATTGCAGAATT |
| | | | | | | | GTCGTGCT |
| | | | | | | | (SEQ ID NO: 5906) |
| | | | | | | | GTGTGGGAGTTGAACGA |
| | | | | | | | CCCTGCTG |
| | | | | | | | (SEQ ID NO: 5907) |
| | | | | | | | GACCCTGTCGGGGCAGT |
| | | | | | | | CAGGGGAC |
| | | | | | | | (SEQ ID NO: 5908) |
| | | | | | | | GAGATTTGATCTCATGC |
| | | | | | | | GAGTCATC |
| | | | | | | | (SEQ ID NO: 5909) |
| | | | | | | | CCTGCCCTGGCTGGCAT |
| | | | | | | | GTGAGAAG |
| | | | | | | | (SEQ ID NO: 5910) |
| | | | | | | | GCTGAGTGTTGGTTCCT |
| | | | | | | | TTTTCTTA |
| | | | | | | | (SEQ ID NO: 5911) |
| 205770_at | GSR | MGC78522 | glutathione | chr8p21.1 | Loss_{—} | 2936 | TACCAACGTCAAAGGCA |
| | | | reductase | | down | | TCTATGCA |
| | | | | | | | (SEQ ID NO: 5912) |
| | | | | | | | GCAATAGCTGCTGGCCG |
| | | | | | | | AAAACTTG |
| | | | | | | | (SEQ ID NO: 5913) |
| | | | | | | | AACTTGCCCATCGACTT |
| | | | | | | | TTTGAATA |
| | | | | | | | (SEQ ID NO: 5914) |
| | | | | | | | CAACATCCCAACTGTGG |
| | | | | | | | TCTTCAGC |
| | | | | | | | (SEQ ID NO: 5915) |
| | | | | | | | TATTCAACGAGCTTTAC |
| | | | | | | | CCCGATGT |
| | | | | | | | (SEQ ID NO: 5916) |
| | | | | | | | TTACCCCGATGTATCAC |
| | | | | | | | GCAGTTAC |
| | | | | | | | (SEQ ID NO: 5917) |
| | | | | | | | AAATGCTGCAGGGTTTT |
| | | | | | | | GCTGTTGC |
| | | | | | | | (SEQ ID NO: 5918) |
| | | | | | | | TTTGACAACACAGTCGC |
| | | | | | | | CATTCACC |
| | | | | | | | (SEQ ID NO: 5919) |
| | | | | | | | TTCACCCTACCTCTTCA |
| | | | | | | | GAAGAGCT |
| | | | | | | | (SEQ ID NO: 5920) |
| | | | | | | | AAGAGCTGGTCACACTT |
| | | | | | | | CGTTGAGA |
| | | | | | | | (SEQ ID NO: 5921) |
| | | | | | | | GAACCAGGAGACACGTG |
| | | | | | | | TGGCGGGC |
| | | | | | | | (SEQ ID NO: 5922) |
| 209234_at | KIF1 B | CMT2, | kinesin family | chr1p36.2 | Loss_{—} | 23095 | TAGCCAAACGGCTGTGC |
| | | CMT2A, | member 1B | | down | | TCAGATGG |
| | | CMT2A1, | | | | | (SEQ ID NO: 5923) |
| | | FLJ23699, | | | | | TGAGCTGAGGTTGGTCT |
| | | HMSNII, | | | | | CTTGCCAA |
| | | KIAA0591, | | | | | (SEQ ID NO: 5924) |
| | | KIAA1448, | | | | | GTTGTTCTTCATGTCTT |
| | | KLP, | | | | | CGAGTTCA |
| | | MGC134844 | | | | | (SEQ ID NO: 5925) |
| | | | | | | | TTTTTTTCATTCTGCCT |
| | | | | | | | ATTCTGGC |
| | | | | | | | (SEQ ID NO: 5926) |
| | | | | | | | ATCAGCTCACTTGAGG |
| | | | | | | | AGTCCCTCA |
| | | | | | | | (SEQ ID NO: 5927) |
| | | | | | | | GGCATCGTCTTAGACTT |
| | | | | | | | TGTGGCTC |
| | | | | | | | (SEQ ID NO: 5928) |
| | | | | | | | TGTGGCTCTAAAGTAC |
| | | | | | | | CTGTCTGTT |
| | | | | | | | (SEQ ID NO: 5929) |
| | | | | | | | ATTTCAAGTCTCTTGTC |
| | | | | | | | ACCATCCT |
| | | | | | | | (SEQ ID NO: 5930) |
| | | | | | | | AAGACTTTGCAAACTGA |
| | | | | | | | TCTCTCCC |
| | | | | | | | (SEQ ID NO: 5931) |
| | | | | | | | GATCTCTCCCCCGTGAA |
| | | | | | | | GGAGTTGA |
| | | | | | | | (SEQ ID NO: 5932) |
| | | | | | | | AACCTTGCGTCACGGAG |
| | | | | | | | CTGTTAGT |
| | | | | | | | (SEQ ID NO: 5933) |
| 202594_at | LEPROT | HSPC112, | leptin | chr8p21.2- | Loss_{—} | 23484 | TATTGCCTAACTTAAGC |
| | L1 | Vps55, | receptor | p21.1 | down | | CATGACTT |
| | | my047 | overlapping | | | | (SEQ ID NO: 5934) |
| | | | transcript- | | | | AATATCGGCGTGTGGCT |
| | | | like 1 | | | | GGAGCCTT |
| | | | | | | | (SEQ ID NO: 5935) |
| | | | | | | | AGTTGGGAACTCTTTGT |
| | | | | | | | GCTTGTGA |
| | | | | | | | (SEQ ID NO: 5936) |
| | | | | | | | GTGCTTGTGATCTACTG |
| | | | | | | | GACTTTTT |
| | | | | | | | (SEQ ID NO: 5937) |
| | | | | | | | CAGGAAGTGCATTCTCT |
| | | | | | | | GGTCCTTC |
| | | | | | | | (SEQ ID NO: 5938) |
| | | | | | | | TCCCTATTTTCTGTTCT |
| | | | | | | | GGATGTCA |
| | | | | | | | (SEQ ID NO: 5939) |
| | | | | | | | AGTGCACTGCTACTGTT |
| | | | | | | | TTATCCAC |
| | | | | | | | (SEQ ID NO: 5940) |
| | | | | | | | GTTTTATCCACTTGGCC |
| | | | | | | | ACAGACTT |
| | | | | | | | (SEQ ID NO: 5941) |
| | | | | | | | AGACTTTTTCTAACAGC |
| | | | | | | | TGCGTATT |
| | | | | | | | (SEQ ID NO: 5942) |
| | | | | | | | GCATTGGCAGCATTGTG |
| | | | | | | | TCTTTGAC |
| | | | | | | | (SEQ ID NO: 5943) |
| | | | | | | | GTGTCTTTGACCTTGTA |
| | | | | | | | TACTAGCT |
| | | | | | | | (SEQ ID NO: 5944) |
| 220183_s_at | NUDT6 | gfg; bFGF; | nudix | chr4q26 | Loss_{—} | 11162 | TATTGGAGACACAGCGG |
| | | FGF-2; gfg- | (nucleoside | | down | | TTCGAGAA |
| | | 1; ASFGF2; | diphosphate | | | | (SEQ ID NO: 5945) |
| | | FGF-AS; | linked moiety | | | | GAGACACAGCGGTTCGA |
| | | FGF2AS | X)-type | | | | GAAGTTTT |
| | | | motif 6 | | | | (SEQ ID NO: 5946) |
| | | | | | | | ACAGCGGTTCGAGAAGT |
| | | | | | | | TTTTGAAG |
| | | | | | | | (SEQ ID NO: 5947) |
| | | | | | | | ATCAGAATTCAGGTCCG |
| | | | | | | | TCCTGAGT |
| | | | | | | | (SEQ ID NO: 5948) |
| | | | | | | | AGGTCCGTCCTGAGTAT |
| | | | | | | | TCGGCAAC |
| | | | | | | | (SEQ ID NO: 5949) |
| | | | | | | | TCCGTCCTGAGTATTCG |
| | | | | | | | GCAACAGC |
| | | | | | | | (SEQ ID NO: 5950) |
| | | | | | | | GTCCTGAGTATTCGGCA |
| | | | | | | | ACAGCACA |
| | | | | | | | (SEQ ID NO: 5951) |
| | | | | | | | GTATTCGGCAACAGCAC |
| | | | | | | | ACAAATCC |
| | | | | | | | (SEQ ID NO: 5952) |
| | | | | | | | TCGGCAACAGCACACAA |
| | | | | | | | ATCCTIGGA |
| | | | | | | | (SEQ ID NO: 5953) |
| | | | | | | | ACAGCACACAAATCCTG |
| | | | | | | | GAGCTTTT |
| | | | | | | | (SEQ ID NO: 5954) |
| | | | | | | | ACACAAATCCTGGAGCT |
| | | | | | | | TTTGGGAA |
| | | | | | | | (SEQ ID NO: 5955) |
| 217022_s_at | IGH@ | IGH; | immunoglobulin | chr14q32.33 | Loss_{—} | 1001265 | TCAAGTGGGAAGAGCGC |
| | | IGH.1@; | heavy locus/// | | down | 83/// | TGTTCAAG |
| | | IGHDY1; | immunoglobulin | | | 28303/// | (SEQ ID NO: 5956) |
| | | MGC72071; | heavy constant | | | 3492/// | GTGGGAAGAGCGCTGT |
| | | MGC88774 | alpha 1/// | | | 3493/// | TCAAGGACC |
| | | | immunoglobulin | | | 3494 | (SEQ ID NO: 5957) |
| | | | heavy constant | | | | ATCCGGAAACACATTCC |
| | | | alpha 2 (A2m | | | | GGCCCGAG |
| | | | marker)/// | | | | (SEQ ID NO: 5958) |
| | | | immunoglobulin | | | | CCCTGAACGAGCTGGTG |
| | | | heavy variable | | | | ACGCTGAC |
| | | | 3/OR16-13/// | | | | (SEQ ID NO: 5959) |
| | | | hypothetical | | | | GAACGAGCTGGTGACGC |
| | | | LOC100126583 | | | | TGACGTGC |
| | | | | | | | (SEQ ID NO: 5960) |
| | | | | | | | CCCCGCGAGAAGTACCT |
| | | | | | | | GACTTGGG |
| | | | | | | | (SEQ ID NO: 5961) |
| | | | | | | | GAGAAGTACCTGACTTG |
| | | | | | | | GGCATCCC |
| | | | | | | | (SEQ ID NO: 5962) |
| | | | | | | | CACCTTCGCTGTGACC |
| | | | | | | | AGCATACTG |
| | | | | | | | (SEQ ID NO: 5963) |
| | | | | | | | TCGCTGTGACCAGCATA |
| | | | | | | | CTGCGCGT |
| | | | | | | | (SEQ ID NO: 5964) |
| | | | | | | | TGACCAGCATACTGCG |
| | | | | | | | CGTGGCAGC |
| | | | | | | | (SEQ ID NO: 5965) |
| | | | | | | | TCAATGTGTCTGTTGTC |
| | | | | | | | ATGGCGGA |
| | | | | | | | (SEQ ID NO: 5966) |
| 212112_s_at | STX12 | RP3- | syntaxin 12 | chr1p35- | Loss_{—} | 23673 | TCACTTGTGGTTCTACA |
| | | 426I6.4, | | p34.1 | down | | TTCCTGGT |
| | | MGC51957, | | | | | (SEQ ID NO: 5967) |
| | | STX13, | | | | | TGAATGTTGCTGTCAAA |
| | | STX14 | | | | | GGGCTGCC |
| | | | | | | | (SEQ ID NO: 5968) |
| | | | | | | | TGCCCCCTACCTTATAA |
| | | | | | | | GGGTTGCT |
| | | | | | | | (SEQ ID NO: 5969) |
| | | | | | | | ATATAAATGTTGTCCAC |
| | | | | | | | TTCCCTTT |
| | | | | | | | (SEQ ID NO: 5970) |
| | | | | | | | CCTTTTCCACAGGCCTA |
| | | | | | | | GAACAGTT |
| | | | | | | | (SEQ ID NO: 5971) |
| | | | | | | | CATAATTTGTTTAGGCT |
| | | | | | | | CCCACATA |
| | | | | | | | (SEQ ID NO: 5972) |
| | | | | | | | GGCCAACAACTTTGGTT |
| | | | | | | | CATCCTTT |
| | | | | | | | (SEQ ID NO: 5973) |
| | | | | | | | GAGGATTTGGCTACCCT |
| | | | | | | | GAGTATAT |
| | | | | | | | (SEQ ID NO: 5974) |
| | | | | | | | TTATATTCATTTCTTCT |
| | | | | | | | GTTCTCCT |
| | | | | | | | (SEQ ID NO: 5975) |
| | | | | | | | AGGGCCCAAATGCATAA |
| | | | | | | | GTTTCTTT |
| | | | | | | | (SEQ ID NO: 5976) |
| | | | | | | | AAGTTTCTTTGCACTGT |
| | | | | | | | TGCACTTA |
| | | | | | | | (SEQ ID NO: 5977) |
| 202033_s_at | RB1CC1 | CC1, | RB1-inducible | chr8q11 | Gain_{—} | 9821 | TGATTTGTCCTGCAGTG |
| | | DRAGOU14, | coiled-coil 1 | | up | | CTCATTCA |
| | | FIP200 | | | | | (SEQ ID NO: 5978) |
| | | | | | | | AACAGCAGGCCATCTTT |
| | | | | | | | TTATGCAA |
| | | | | | | | (SEQ ID NO: 5979) |
| | | | | | | | ACTTCACTGGTGTACAT |
| | | | | | | | CGTTTACT |
| | | | | | | | (SEQ ID NO: 5980) |
| | | | | | | | TCGTTTACTTTTTAACT |
| | | | | | | | GGCTTCAT |
| | | | | | | | (SEQ ID NO: 5981) |
| | | | | | | | AGAAATGCGGACCAAAC |
| | | | | | | | TACTTCAT |
| | | | | | | | (SEQ ID NO: 5982) |
| | | | | | | | ACTACTTCATTTTCTCA |
| | | | | | | | AAGGGCAT |
| | | | | | | | (SEQ ID NO: 5983) |
| | | | | | | | GCATACCTTGTGCATTG |
| | | | | | | | TGGCTTAT |
| | | | | | | | (SEQ ID NO: 5984) |
| | | | | | | | GATGAGCCATATTAATT |
| | | | | | | | GCCTGTTA |
| | | | | | | | (SEQ ID NO: 5985) |
| | | | | | | | GTTATTATTACCAGCATT |
| | | | | | | | TGTCCTT |
| | | | | | | | (SEQ ID NO: 5986) |
| | | | | | | | ATACTTGCACTCTTTAA |
| | | | | | | | CACATTCT |
| | | | | | | | (SEQ ID NO: 5987) |
| | | | | | | | AGAGGAGTGTTATTGCA |
| | | | | | | | TGCTGATA |
| | | | | | | | (SEQ ID NO: 5988) |
| 212096_s_at | MTUS1 | ATIP, | mitochondrial | chr8p22 | Loss_{—} | 57509 | TGGGTCATTTACATGTA |
| | | DKFZp586D | tumor | | down | | CACTACAT |
| | | 1519, | suppressor 1 | | | | (SEQ ID NO: 5989) |
| | | DKFZp686F | | | | | GATACTCATGTTGCATG |
| | | 20243, | | | | | TTCTTTTA |
| | | FLJ14295, | | | | | (SEQ ID NO: 5990) |
| | | KIAA1288, | | | | | TAGTGATTTTGTGTCTT |
| | | MP44, | | | | | AAGTCTTT |
| | | MTSG1 | | | | | (SEQ ID NO: 5991) |
| | | | | | | | TAAGTCTTTAACTTCCA |
| | | | | | | | ATACTTCA |
| | | | | | | | (SEQ ID NO: 5992) |
| | | | | | | | GTATGTAACCTTCCATG |
| | | | | | | | TTTGCTTC |
| | | | | | | | (SEQ ID NO: 5993) |
| | | | | | | | GGAAATGTAGGTTCACT |
| | | | | | | | GCCACTTC |
| | | | | | | | (SEQ ID NO: 5994) |
| | | | | | | | GGTTCACTGCCACTTCA |
| | | | | | | | TGAGATAT |
| | | | | | | | (SEQ ID NO: 5995) |
| | | | | | | | CATGAGATATCTCTGCT |
| | | | | | | | CACGCTTC |
| | | | | | | | (SEQ ID NO: 5996) |
| | | | | | | | CAAAGTTGGGTTTGCC |
| | | | | | | | ATTCATCCC |
| | | | | | | | (SEQ ID NO: 5997) |
| | | | | | | | ATGGTAAATCTTGTGTT |
| | | | | | | | GTTCCCTG |
| | | | | | | | (SEQ ID NO: 5998) |
| | | | | | | | TGTCCTCCGTATTACGT |
| | | | | | | | GACCGGCA |
| | | | | | | | (SEQ ID NO: 5999) |
| 212295_s_at | SLC7A1 | ERR; | solute carrier | chr13q12- | Gain_{—} | 6541 | TTCCTGCTTAAGTTGTG |
| | | ATRC1; | family 7 | q14 | up | | CCTTTCAG |
| | | CAT-1; | (cationic | | | | (SEQ ID NO: 6000) |
| | | HCAT1; | amino acid | | | | AAGTTGTGCCTTTCAGC |
| | | REC1L | transporter, | | | | TTCAATGA |
| | | | y+ system), | | | | (SEQ ID NO: 6001) |
| | | | member 1 | | | | GAATAGCTGTGGTTTCT |
| | | | | | | | CCAGTAAA |
| | | | | | | | (SEQ ID NO: 6002) |
| | | | | | | | TCTTCTACTGAACATGG |
| | | | | | | | AGCCATTA |
| | | | | | | | (SEQ ID NO: 6003) |
| | | | | | | | AAATCAGGTTGGTCTCT |
| | | | | | | | GCTGGGTC |
| | | | | | | | (SEQ ID NO: 6004) |
| | | | | | | | GACAAGTGTAGTTGTCG |
| | | | | | | | TCCACCAC |
| | | | | | | | (SEQ ID NO: 6005) |
| | | | | | | | TCGTCCACCACCTTTCA |
| | | | | | | | AAAAATGT |
| | | | | | | | (SEQ ID NO: 6006) |
| | | | | | | | CCTTTTTGCTGACAACA |
| | | | | | | | CTGTGTAC |
| | | | | | | | (SEQ ID NO: 6007) |
| | | | | | | | ACACTGTGTACATTGAC |
| | | | | | | | CACTTCCT |
| | | | | | | | (SEQ ID NO: 6008) |
| | | | | | | | ACCACTTCCTACCATAC |
| | | | | | | | TTTATGTT |
| | | | | | | | (SEQ ID NO: 6009) |
| | | | | | | | GGTACATTATCTCATGC |
| | | | | | | | TTCTGCAA |
| | | | | | | | (SEQ ID NO: 6010) |
| 219822_at | MTRF1 | RF1; MRF1; | mitochondrial | chr13q14.1- | Gain_{—} | 9617 | TTGGACCCCAAGGATTT |
| | | MTTRF1; | translational | q14.3 | up | | GCGAATAG |
| | | MGC47721 | release | | | | (SEQ ID NO: 6011) |
| | | | factor 1 | | | | GATACATTTCGAGCCAA |
| | | | | | | | AGGAGCAG |
| | | | | | | | (SEQ ID NO: 6012) |
| | | | | | | | TGATAGTGCCGTCAGAC |
| | | | | | | | TTGTCCAC |
| | | | | | | | (SEQ ID NO: 6013) |
| | | | | | | | CCACATCCCCACAGGGC |
| | | | | | | | TAGTAGTA |
| | | | | | | | (SEQ ID NO: 6014) |
| | | | | | | | GCCTTTCGTGTGTTGAG |
| | | | | | | | AGCTAGAC |
| | | | | | | | (SEQ ID NO: 6015) |
| | | | | | | | GAGCTAGACTCTACCAG |
| | | | | | | | CAGATTAT |
| | | | | | | | (SEQ ID NO: 6016) |
| | | | | | | | AGAGCCCAGTCAGAGCG |
| | | | | | | | AATTCGGA |
| | | | | | | | (SEQ ID NO: 6017) |
| | | | | | | | TCGGACATATAATTTCA |
| | | | | | | | CCCAGGAT |
| | | | | | | | (SEQ ID NO: 6018) |
| | | | | | | | GTCAGTGACCACAGGAT |
| | | | | | | | AGCATATG |
| | | | | | | | (SEQ ID NO: 6019) |
| | | | | | | | GGGAAGGGCCTGGATCA |
| | | | | | | | GCTAATTC |
| | | | | | | | (SEQ ID NO: 6020) |
| | | | | | | | GCTGAACTTTTGGATGA |
| | | | | | | | ACACCTTA |
| | | | | | | | (SEQ ID NO: 6021) |
| 209384_at | PROSC | FLJ11861 | proline | chr8p11.2 | Loss_{—} | 11212 | TTTTTCCTCTGGGACCT |
| | | | synthetase | | down | | ACAGTGAT |
| | | | cotranscribed | | | | (SEQ ID NO: 6022) |
| | | | homolog | | | | CTCCTCCACTATAATCC |
| | | | (bacterial) | | | | TCTTTAGG |
| | | | | | | | (SEQ ID NO: 6023) |
| | | | | | | | AACCCAGTGAATCTCAT |
| | | | | | | | TACTCCTA |
| | | | | | | | (SEQ ID NO: 6024) |
| | | | | | | | GCCTTTTCAGGCACATG |
| | | | | | | | GTTTCAAC |
| | | | | | | | (SEQ ID NO: 6025) |
| | | | | | | | GACATTCCTTGTCCTGA |
| | | | | | | | GGAGCACT |
| | | | | | | | (SEQ ID NO: 6026) |
| | | | | | | | TTCCAGGCATAGTTACA |
| | | | | | | | GCTTCCCC |
| | | | | | | | (SEQ ID NO: 6027) |
| | | | | | | | GCTTCCCCACTGTATTT |
| | | | | | | | ACAAGCCA |
| | | | | | | | (SEQ ID NO: 6028) |
| | | | | | | | TGTGCAACTCTTCTGGA |
| | | | | | | | TCATTAAT |
| | | | | | | | (SEQ ID NO: 6029) |
| | | | | | | | AAATGGCTTCAGTATGG |
| | | | | | | | CTTGTTTT |
| | | | | | | | (SEQ ID NO: 6030) |
| | | | | | | | TCCAGATGGCTTTTTCT |
| | | | | | | | CTTATTTT |
| | | | | | | | (SEQ ID NO: 6031) |
| | | | | | | | GAAGCCCCAGTCTTTGA |
| | | | | | | | TTTTACAG |
| | | | | | | | (SEQ ID NO: 6032) |

All gene probe set identification numbers and Entrez Gene identification numbers of Table 4 are hereby incorporated by reference in their entirety

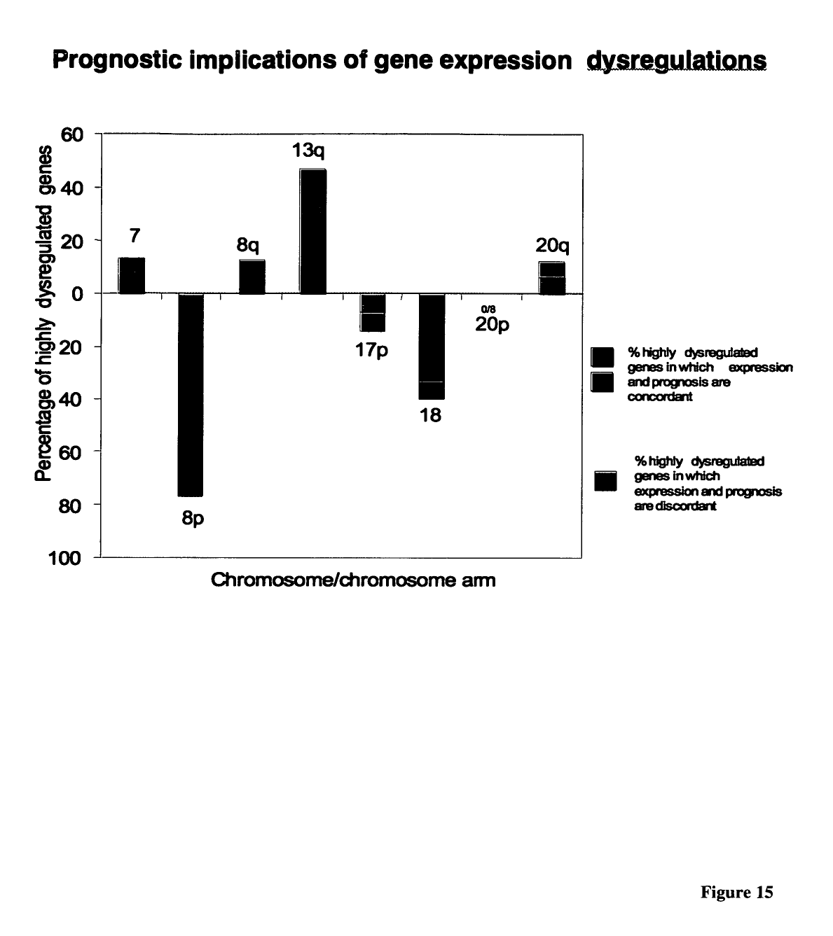

The concordance between dysregulation and prognostic effect is highly evident in the 8p arm (FIG. 15). The KM plots for the 10 dysregulated genes in the 8p arm (Table 4) are illustrated in FIG. 16. Interestingly, the 20q arm, which is the most highly dysregulated chromosome arm (FIG. 13), has only one gene (TMEPAI) considered a good prognostic indicator. Among the 8p genes in the list is MTUS1, a putative tumor suppressor (Seibold et al., “Identification of a New Tumor Suppressor Gene Located at Chromosome 8p21.3-22” Faseb J 17:1180-1182 (2003), which is hereby incorporated by reference in its entirety) previously shown to be downregulated in colorectal cancer (Lee et al., “Differential Expression in Normal-Adenoma-Carcinoma Sequence Suggests Complex Molecular Carcinogenesis in Colon” Oncol Rep 16:747-754 (2006), which is hereby incorporated by reference in its entirety). The downregulation of PCM1 has been detected in both ovarian cancer (Pils et al., “Five Genes from Chromosomal Band 8p22 are Significantly Down-Regulated in Ovarian Carcinoma: N33 and EFA6R Have a Potential Impact on Overall Survival” Cancer 104:2417-2429 (2005), which is hereby incorporated by reference in its entirety) and breast cancer (Armes et al., “Candidate Tumor-Suppressor Genes on Chromosome Arm 8p in Early-Onset and High-Grade Breast Cancers” Oncogene 23:5697-5702 (2004), which is hereby incorporated by reference in its entirety). Recent studies suggest that the downregulation of ADAMDEC1 and EPHX2 may be directly associated with metastasis in colon cancer (Macartney-Coxson et al., “Metastatic Susceptibility Locus, an 8p Hot-Spot for Tumor Progression Disrupted in Colorectal Liver Metastases: 13 Candidate Genes Examined at the DNA, mRNA and Protein Level” BMC Cancer 8:18 (2008), which is hereby incorporated by reference in its entirety), and in breast cancer (Thomassen et al., “Gene Expression Meta-Analysis Identifies Chromosomal Regions and Candidate Genes Involved in Breast Cancer Metastasis” Breast Cancer Res Treat 113(2):239-49 (2008), which is hereby incorporated by reference in its entirety) respectively.

### Example 11

### Lecithin:Retinol Acyl Transferase Gene Promoter Methylation Analysis

Sodium bisulfite has been widely used to distinguish 5-methylcytosine from cytosine. Bisulfite converts cytosine into uracil via a deamination reaction while leaving 5-methylcytosine unchanged. Genomic DNAs extracted from colon tumor samples were used in this study. Typically, 1˜0.5 μg genomic DNA in a volume of 40 μl was incubated with 0.2N NaOH at 37° C. for 10 minutes. Next, 30 μl of 10 mM hydroquinone and 520 μl of 3M sodium bisulfite were added to the reaction. Sodium bisulfite (3M) was made with 1.88 g sodium bisulfite (Sigma Chemicals, ACS grade) dissolved in a final total of 5 ml deionized water at pH 5.0. The bisulfite/DNA mixture was incubated for 16 hours in a DNA thermal cycler (Perkin Elmer Cetus), cycling between 50° C. for 20 minutes and 85° C. for 15 seconds. The bisulfite treated DNA was desalted using MICROCON centrifugal filter devices (Millipore, Bedford, Mass.) or, alternatively, was cleaned with Wizard DNA clean-up kit (Promega, Madison, Wis.). The eluted DNA was incubated with one-tenth volume of 3N NaOH at room temperature for 5 minutes before ethanol precipitation. The DNA pellet was then resuspended in 20 μl deionized H₂O and stored at 4° C. until PCR amplification.

### Example 12

### Multiplex PCR Amplification

Two promoter regions of the LRAT gene were simultaneously amplified in a multiplex fashion. The multiplex PCR has two stages, namely a gene-specific amplification (stage one) and a universal amplification (stage two). The PCR primers are shown in Table 5.

TABLE 5
Primer Sequences for LRAT Analysis

| | | | CONC. |
|---|---|---|---|
| PRIMERS | | SEQUENCE (5′ to 3′) | in PCR |
| LRAT | SEQ ID | CGCTGCCAACTACCGCACATCTTAT | 1.25 pmol |
| RP1 | NO: 6033 | TTTTTATTGTGGTTTGGTTGAGTC | |
| LRAT | SEQ ID | CGCTGCCAACTACCGCACATCACCT | 1.25 pmol |
| FP1 | NO: 6034 | CCAACATAAAATTCTTCATCCTAC | |
| LRAT | SEQ ID | CGCTGCCAACTACCGCACATCATAAT | 2.5 pmol |
| RP3 | NO: 6035 | CGTGTTGTTTATATGATGTTCGATA | |
| LRAT | SEQ ID | CGCTGCCAACTACCGCACATCCACAA | 2.5 pmol |
| FP3 | NO: 6036 | CAAACTATAAAAAATAAAACCCAAC | |
| UniB2 | SEQ ID | CGCTGCCAACTACCGCACATC | 12.5 pmol |
| | NO: 6037 | | |

The gene-specific PCR primers were designed such that the 3′ sequence contains a gene-specific region and the 5′ region contains an universal sequence. The gene specific primers design allows hybridization to promoter regions containing as few CpG sites as possible. For primers that inevitably include one or more CpG dinucleotides, the nucleotide analogs, K and P, which can hybridize to either C or T nucleotides or G or A nucleotides, respectively, can be included in the primer design. To reduce the cost of primer synthesis, PCR primers were designed without nucleotide analogs and using nucleotides G to replace K (purine derivative) and T to replace P (pyrimidine derivative), respectively. This type of primer design favors pairing to DNA that was initially methylated, although it also allows the mismatch pairing of G/T when the original DNA was unmethylated at that site. The ethidium bromide staining intensity of PCR amplicons separated by the agarose gel electrophoresis, demonstrated that this primer design was as robust as using analogs-containing primers.

In the first stage, the multiplex PCR reaction mixture (12.40 consisted of 0.5 μl bisulfite modified DNA, 400 μM of each dNTP, 1× AmpliTaq Gold PCR buffer, 4 mM MgCl2, and 1.25 U AmpliTaq Gold polymerase. The gene-specific PCR primer concentrations are listed in the Table 5. Mineral oil was added to each reaction before thermal cycling. The PCR procedure included a pre-denaturation step at 95° C. for 10 minutes, 15 cycles of three-step amplification with each cycle consisting of denaturation at 94° C. for 30 second, annealing at 60° C. for 1 minute, and extension at 72° C. for 1 minute. A final extension step was at 72° C. for 5 minutes.

The second stage of multiplex PCR amplification was primed from the universal sequences (UniB) located at the extreme 5′ end of the gene-specific primers. The second stage PCR reaction mixture (12.50 consisted of 400 μM of each dNTP, 1× AmpliTaq Gold PCR buffer, 4 mM MgCl2, 12.5 μmol universal primer B (UniB) and 1.25 U AmpliTaq Gold polymerase. The UniB PCR primer sequence is listed in the Table 5. The 12.5 μl reaction mixtures were added through the mineral oil to the finished first stage PCR reactions. The PCR procedure included a pre-denaturation step at 95° C. for 10 minutes, 30 cycles of three-step amplification with each cycle consisting of denaturation at 94° C. for 30 second, annealing at 55° C. for 1 minute, and extension at 72° C. for 1 minute. A final extension step was at 72° C. for 5 minutes.

After the two-stage PCR reaction, 1.25 μl Qiagen Proteinase K (approximately 20 mg/ml) was added to the total 25 μl reaction. The Proteinase K digestion condition consisted of 70° C. for 10 minutes and 90° C. for 15 minutes.

### Example 13

### Ligase Detection Reaction and Hybridization to Universal Array

Ligation detection reactions were carried out in a 20 μl volume containing 20 mM Tris-HCl pH 7.6, 10 mM MgCl2, 100 mM KCl, 20 mM DTT, 1 mM NAD, 50 fmol wild-type Tth ligase, 500 fmol each of LDR probes, 5-10 ng each of the PCR amplicons. The Tth ligase can be diluted in a buffer containing 15 mM Tris-HCl pH 7.6, 7.5 mM MgCl2, 0.15 mg/ml BSA. To ensure the scoring accuracy of a LRAT promoter methylation status, 30 LDR probes were designed to interrogate the methylation levels of ten CpG dinucleotide sites within the PCR amplified regions. Two discriminating LDR probes and one common LDR probe were designed for each of the CpG sites. The LDR probe mix contains 60 discriminating probes (30 probes for each channel) and 10 common probes (Table 6). The reaction mixtures were pre-heated for 3 minutes at 95° C., and then cycled for 20 rounds of 95° C. for 30 seconds and 60° C. for four minutes.

The ligation detection reaction (20 μl) was diluted with equal volume of 2× hybridization buffer (8×SSC and 0.2% SDS), and denatured at 95° C. for 3 minutes then plunged on ice. The Universal Arrays (Amersham Biosciences, Piscataway, N.J.) were assembled with ProPlate slide modules (Grace Bio-Labs, Bend, Oreg.) and filled with the 40 μl denatured LDR mixes. The assembled arrays were incubated in a rotating hybridization oven for 60 minutes at 65° C. After hybridization, the arrays were rinsed briefly in 4×SSC and washed in 2×SSC, 0.1% SDS for 5-10 minutes at 63.5° C. The fluorescent signals were measured using a ProScanArray scanner (Perkin Elmer, Boston, Mass.).

TABLE 6
Probe Sequences for Ligase Detection Reaction

| PRIMERS | SEQUENCE (5′ to 3′) | |
|---|---|---|
| LRAT 53T | (Cy5) TGTTATTT TT TAT TGT GGT TTG GTT GAG TTG GTT | (SEQ ID NO: 6038) |
| LRAT 172Tb | (Cy5) TTGTTTTTTTTTTTT TTT AGT GGT TGT ATT TTG TGT T | (SEQ ID NO: 6039) |
| LRAT 218T | (Cy5) TTTTGTC GGA GTG GTA TTG GTA TTT TTT TAA GAT | (SEQ ID NO: 6040) |
| LRAT 222T | (Cy5) GGTTTGTAGG TGA GTA GTA GTG TAG TAT TTT TGT TTG GT | (SEQ ID NO: 6041) |
| LRAT 242Tb | (Cy5) GTGTAGTATTTTTGT TTG GTG AGT TTA ATT TGT TTA GTT T | (SEQ ID NO: 6042) |
| LRAT 263T | (Cy5) TTAATTTGTT TAG TTT GGT TTT TGT TGG AGT GGT ATT | (SEQ ID NO: 6043) |
| LRAT2nd 40T | (Cy5) TTTATATGA TGT TTG ATA TTT TGT TGG TTT TGA TAG AT | (SEQ ID NO: 6044) |
| LRAT2nd 91T | (Cy5) GTAGAAGGTGG TTT TTA ATA AGT GTT TTA TTT TGG GT | (SEQ ID NO: 6045) |
| LRAT2nd 145T | (Cy5) GTATTCGCG TGG ATA TAG TGG AGG ATT TTG TTT AT | (SEQ ID NO: 6046 |
| LRAT2nd 218T | (Cy5) TAGAAAAAGGTATTG TTT AAT GAG GAG GTG GTG T | (SEQ ID NO: 6047) |
| LRAT 53C | (Cy3) ATTTTTTATTGTGGTTTGGTTGAGTCGGTC | (SEQ ID NO: 6048) |
| LRAT 172C | (Cy3) TTTTTTTTTAGCGGTCGTATTTTGCGTC | (SEQ ID NO: 6049) |
| LRAT 218C | (Cy3) TGTCGGAGTGGTATCGGTATTTTTTTAAGAC | (SEQ ID NO: 6050) |
| LRAT 222C | (Cy3) GTAGGTGAGTAGTAGCGTAGTATTTTTGTTCGGC | (SEQ ID NO: 6051) |
| LRAT 242C | (Cy3) TTTTGTTCGGCGAGTTTAATTTGTTTAGTTC | (SEQ ID NO: 6052) |
| LRAT 263C | (Cy3) TGTTTAGTTCGGTTTTTGTCGGAGTGGTATC | (SEQ ID NO: 6053) |
| LRAT2nd 40C | (Cy3) ATATGATGTTCGATATTTTGTTGGTTTTGATAGAC | (SEQ ID NO: 6054) |
| LRAT2nd 91C | (Cy3) GGTGGTTTTTAATAAGCGTTTTATTTTGGGC | (SEQ ID NO: 6055) |
| LRAT2nd 145C | (Cy3) TCGCGTGGATATAGTGGAGGATTTCGTTTAC | (SEQ ID NO: 6056) |
| LRAT2nd 218C | (Cy3) GGTATTGTTTAACGAGGAGGTGGCGC | (SEQ ID NO: 6057) |
| LRAT 53C (Z18) | pGTTAGGTTTCGTTGTTTTTTTTTGTTTTTTTTTTTTTTTAGGGAGGCTGCTGTC | (SEQ ID NO: 6058) |

| | | |
|---|---|---|
| | CTTTCGATCA | |
| 2LRAT 172C | pGTATTTTATTTGGTTTGTAGGTGAGTAGTAGCGTAGTATTTTTG | (SEQ ID NO: 6059) |
| (Z19) | ACAGCGTGTTCGTTGCTTGCATCA | |
| 3LRAT 222Cc | pGAG TTT AAT TTG TTT AGT TCG GTT TTT GTT | (SEQ ID NO: 6060) |
| (Z31) | GGATTGCGGGAACTCACGAGGTCGTAT | |
| 4LRAT 242Cb | pGGTTTTTGTCGGAGTGGTATTGGTATTTTTTTAATGGCGATGGTCCACTCGCAA | (SEQ ID NO: 6061) |
| (Z20) | TCA | |
| 5LRAT 263Cb | pGGT ATT TTT TTA AGA CGT TTT TTT TTT TGT AGG | (SEQ ID NO: 6062) |
| (Z32) | ATGGCACGGCTCGATAGGTCAAGCTTT | |
| 6LRAT 218C | pGTTTTTTTTTTTGTAGGATGAAGAATTTTATGTTGGAGGCATCGCACTTCGCTT | (SEQ ID NO: 6063) |
| (Z30) | TGGCTGATT | |
| 7LRAT2nd 40C | pGAT ATG GGG TGT ACG TAG AAG GTG GTT | (SEQ ID NO: 6064) |
| (Z10) | TTTACAAGGCACGTCCCAGACGCATCAA | |
| 8LRAT2nd 91C | pGTT ATT GTT AAA GTG GTT AGT ATT TGC GTG GAT ATA | (SEQ ID NO: 6065) |
| (Z11) | GTGGCACGGGAGCTGACGACGTGTCAA | |
| 9LRAT2nd 145C | pGGAGTTAATATTTTGGTTAATTATTTGGACGAGTTTTTTTAGACGCACCGCAAC | (SEQ ID NO: 6066) |
| (Z12) | AGGCTGTCAA | |
| 10LRAT2nd 218C | pGGAGGGTTGAAAAGTTGTTGGGTTTTATTTTTCATCGCTGCAAGTACCGCACTC | (SEQ ID NO: 6067) |
| (Z13) | AA | |

### Example 14

### Determination of Cytosine Methylation Levels at CpG Dinucleotide Sites

LDR is a single tube multiplex reaction with three probes interrogating each of the selected CpG sites. LDR products are captured on a Universal microarray using the ProPlate system (Grace BioLabs) where 64 hybridizations (four slides with 16 sub-arrays each) are carried out simultaneously. Each slide is scanned using a Perkin Elmer ProScanArray (Perkin Elmer, Boston, Mass.) under the same laser power and PMT within the linear dynamic range. The Cy3 and Cy5 dye bias were determined by measuring the fluorescence intensity of an equal quantity of Cy3 and Cy5 labeled LDR probes manually deposited on a slide surface. The fluorescence intensity ratio (W=ICy3/ICy5) was used to normalize the label bias when calculating the methylation ratio Cy3/(Cy3+Cy5). The methylation standard curves for each interrogated CpG dinucleotide were established using various combinations of in vitro methylated and unmethylated normal human lymphocyte genomic DNAs. The methylation levels of six CpG dinucleotides in the 5′-UTR regions were averaged and used to determine the overall promoter methylation status of LRAT gene.

### Example 15

### Quantitative Aspect of Bisulfite/PCR-PCR/LDR/Universal Array

Because PCR primer and LDR probe design does not bias amplification or detection of methylation status, independent of methylation status of neighboring CpG dinucleotides (i.e. by using nucleotide analogues or degenerate bases within the primer designs), it is possible to quantify methylation status of given CpG sites in the genome.

To demonstrate that the assay is quantitative, genomic DNA in vitro methylated with SssI methylase was mixed with normal human lymphocytes DNA (carrying unmethylated alleles), such that the test samples contained 0%, 20%, 40%, 60%, 80%, and 100% of methylated alleles and these mixtures were subjected to Bisulfite-PCR/LDR/Universal Array analysis. The fluorescence intensity is presented by Cy3 (methylated alleles) or Cy5, (unmethylated alleles) on each double spotted zipcode addresses. The average fluorescence intensity of two duplicated spots was used to calculate the methylation ratio of each analyzed cytosine using the formula Cy3average/(Cy3 average+Cy5 average).

The measured methylation ratios of each interrogate cytosine was plotted against the methylation levels of mixed genomic DNAs. The R² values (correlation coefficient) of these experiments are between 0.97 and 0.89, which demonstrates the linearity of the described assay. Such standard curves can be used as reference points for further measurements done in clinical samples. Similar standard curves were also established for genes such as p16INK4a, p14ARF, TIMP3, APC, RASSF1, ECAD, MGMT, DAPK, GSTP1 and RARβ (Cheng et al., “Multiplexed Profiling of Candidate Genes for CpG Island Methylation Status Using a Flexible PCR/LDR/Universal Array Assay,” Genome Res. 16(2):282-289 (2006), which is hereby incorporated by reference in its entirety). In “100%” in vitro methylated DNA sample, the Cy3average/(Cy3 average+Cy5 average) ratios of the investigated CpG sites were between 0.6 and 0.9. This observation suggested that in vitro methylation is not fully efficient due to sequence context variation of each CpG site. This analysis also confirmed the different percentage of methylation at each CpG dinucleotide and suggested that methylation level is not 100% at each CpG site in cell line DNA (Cheng et al., “Multiplexed Profiling of Candidate Genes for CpG Island Methylation Status Using a Flexible PCR/LDR/Universal Array Assay,” Genome Res. 16(2):282-289 (2006), which is hereby incorporated by reference in its entirety). By comparing the ratio of (methylated):(methylated+unmethylated) DNA in different cell lines, one can extrapolate the CpG methylation level at a given position. Overall, the data demonstrate that the bisulfite-PCR/LDR/Universal Array approach is a quantitative method for the measurement of DNA methylation.

### Example 16

### Tumor Specific LRAT Promoter Hypermethylation

Since aberrant DNA methylation may also result from aging, it is necessary to identify a promoter region where its methylation is disease specific. To demonstrate LRAT promoter region methylation is tumor specific, CRC tumor samples (n=133) and the adjacent normal tissues (n=69) were analyzed using bisulfite/PCR-PCR/LDR/Universal Array approach. For each clinical sample, the methylation levels of ten CpG dinucleotide sites residing in the 5′-UTR (CpG sites 1-6) and exon-1 (CpG sites 7-10) regions of LRAT promoter were interrogated. Since the tumor (disease) specific aberrant methylation was identified in the 5′-UTR, the methylation levels of CpG sites 1-6 were averaged (the mean value) to determine the overall promoter methylation status. A promoter with a mean value of methylation signal intensity greater than 0.2 was scored as hypermethylated (methylation score 1), while a mean value equal to or less than 0.2 was scored as unmethylated (methylation score 0). This approach allowed a simple scoring system to use quantitative methylation data from multiple representative CpG sites across a larger DNA sequence region. Such quantitative reports give non-ambiguous and repeatable results of study DNA methylation.

A series of 133 CRC patient samples from Memorial-Sloan Kettering Cancer Center tumor bank were subject to bisulfite/PCR-PCR/LDR/Universal Array analysis. The methylation levels of ten CpG dinucleotide sites in the LRAT promoter region were determined for each CRC sample. The average methylation level of CpG sites 1-6 was used to score the overall LRAT promoter methylation status. A hypermethylated promoter was defined as having an average methylation level greater than 0.2.

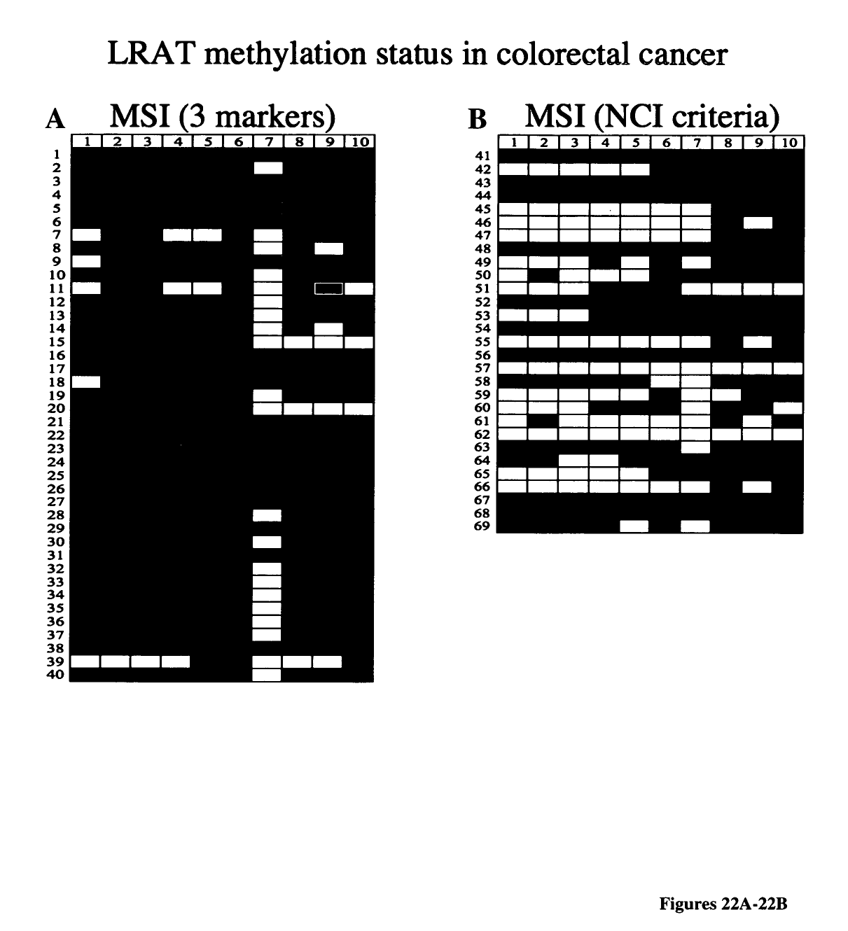

LRAT promoter hypermethylation in CRCs was initially studied in microsatellite instability (MSI) tumors that often show multiple hypermethylated genes. LRAT hypermethylation was found in 36 of 40 MSI samples (90%) and was confirmed using methylation specific PCR (FIG. 22A). Since the MSI patients typically have a better clinical outcome and MSI accounts for only 10-15% of sporadic CRCs, the frequency of aberrant LRAT hypermethylation in the majority of CRC instances was examined in 81 microsatellite stable (non-MSI) colorectal samples (FIG. 22B).

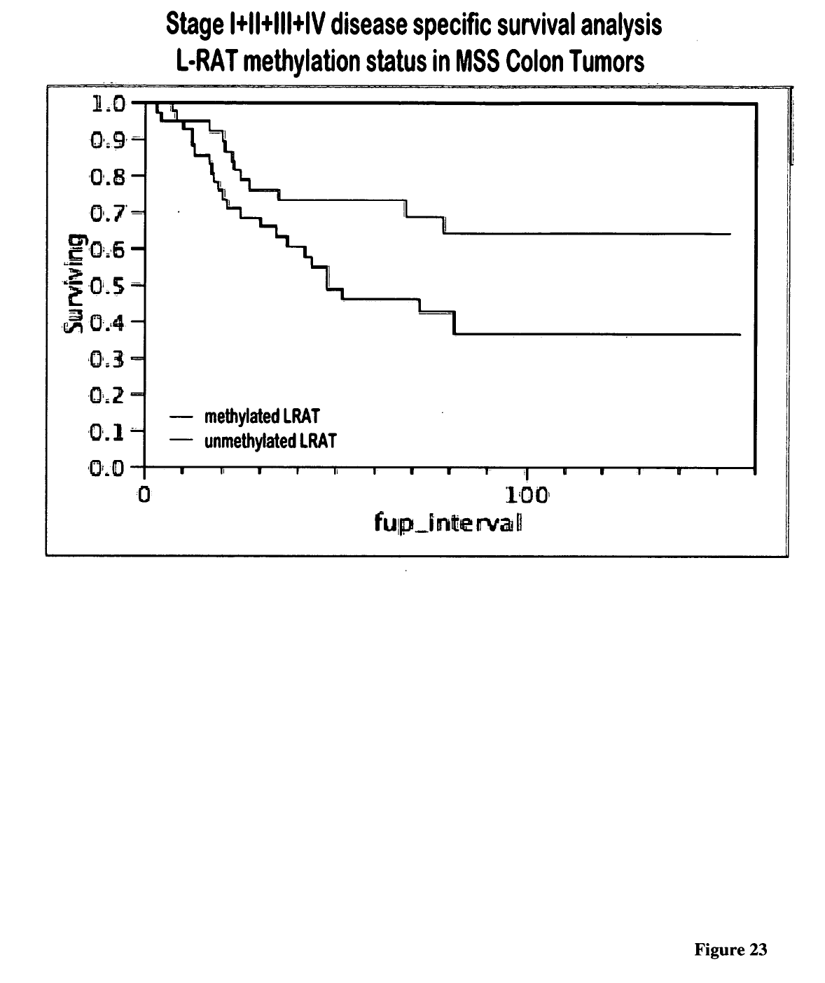

LRAT promoter methylation is significantly associated with increased survival for all sporadic, non-MSI CRC patients. When all four CRC stages were considered, patients with LRAT promoter hypermethylation had a better disease-specific survival rate than patients with unmethylated promoter (FIG. 23). Only 12 of 39 (30.8%) individuals with LRAT promoter hypermethylation had died within the study period, whereas 23 of 42 (54.8%) individuals with unmethylated LRAT promoter had died. Log rank test was used to compare the two survival curves produced from methylated and unmethylated LRAT groups (p=0.0296).

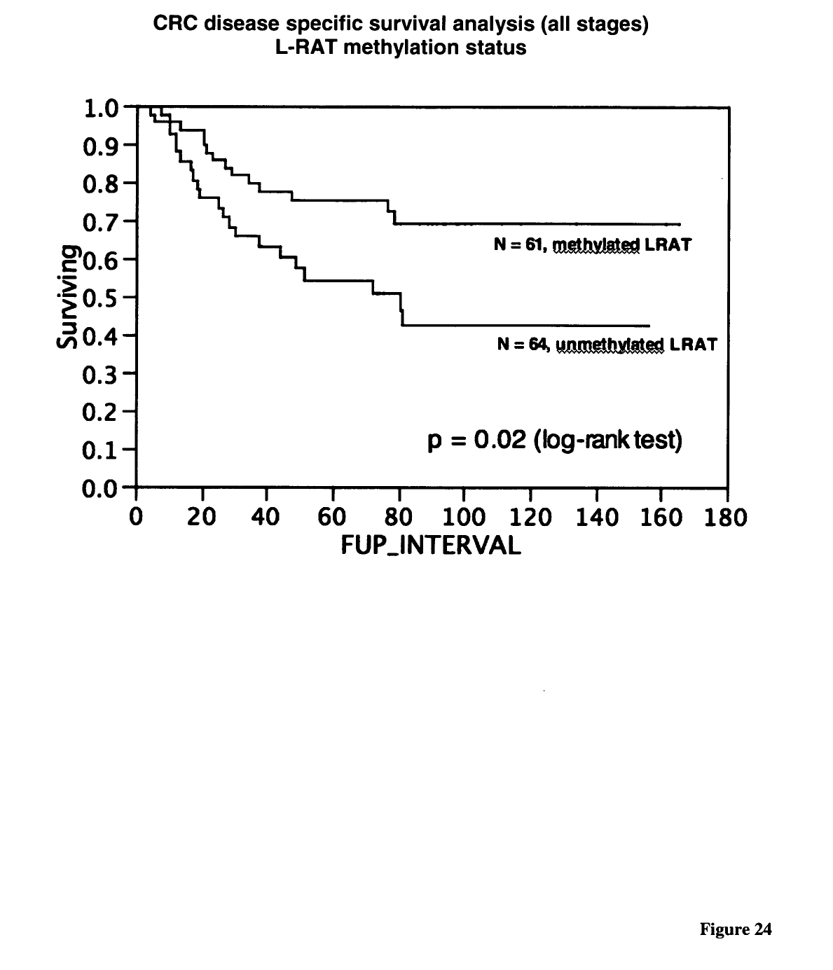

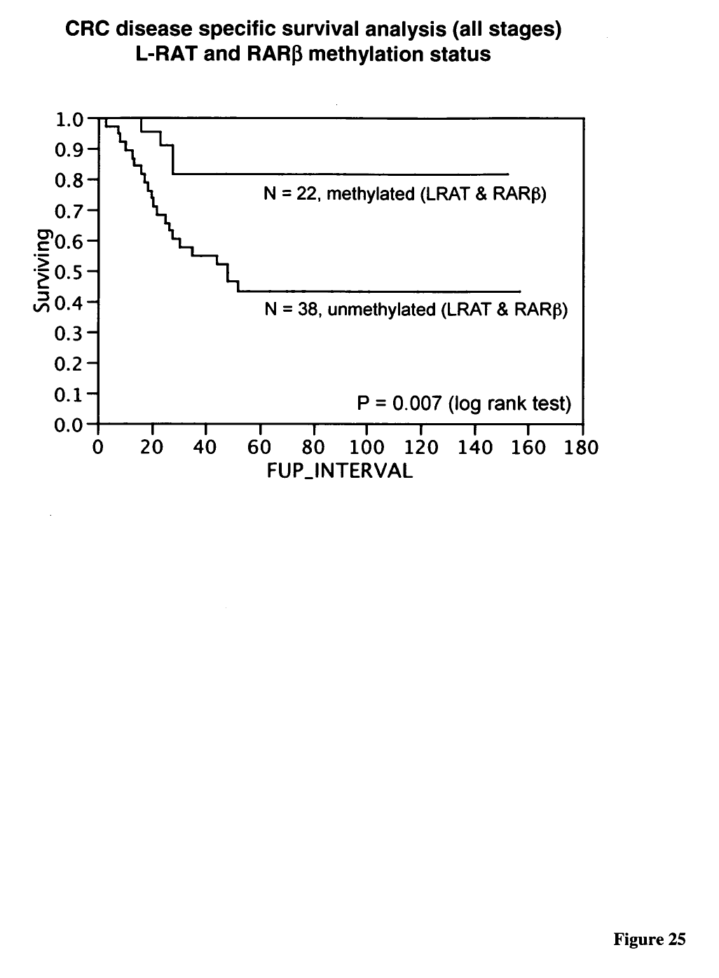

In a validation study, Kaplan-Meier survival analysis was carried out on an additional 44 non-MSI colorectal samples (total n=125) (FIG. 24) and a similar survival curve (p=0.02) was observed. In a subset of 60 colorectal tumor samples, analysis of methylation status of LRAT and retinoic acid receptor β(RARβ) revealed that promoter hypermethylation at both genes correlates with better prognosis (p=0.007, FIG. 25). This observation suggests that the association between LRAT methylation silencing and better prognosis may represent a RARβ independent pathway to the inhibition of tumorigenesis

Since the MSI patients typically have a better survival and clinical outcome, Kaplan-Meier survival analysis was performed on patients with non-MSI genotype. Survival was measured from the date of resection of colorectal cancer to the date of death, the completion of 5 years of follow-up, or the last clinical review before April 2006. Only cancer-related deaths were analyzed as events. A p-value of less than 0.05 was considered as statistical significance.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method for determining the prognosis of a subject having colon cancer, said method comprising:
obtaining a biological sample from the subject;
detecting expression levels of at least five genes selected from a group of 176 genes informative of colon cancer prognosis consisting of ACSL4, RQCD1, AA058828*, AIP, AKR1A1, AP3D1, ARL2BP, ARL4A, ARL61P4, OGFOD2, ASNA1, ATP5B, C12orf52, C19orf36, C1GALT1, C1orf144, C5orf23, C6orf15, C7orf10, C8orf70, CALML4, CASP1, CCNA2, CCT2, CDC42BPA, AK023058*, CDR2L, CFB, CHST12, CLN5, CMPK1, CNOT7, CNPY2, COBL, COMMD4, COX5A, CXCL11, CYB561, CYB5B, DAZAP2, DDX23, DENND2A, DENND2D, DHX15, AL359599*, DND1, DOCK9, EGFR, ELP3, ERP29, ETV1, FAM82C, FDFT1, FKBP14, FLJ10357, FRYL, GALNS, GCHFR, GHITM, GLS, GPR177, GRB10, GREM2, GRHPR, GRP, GSR, GSTA1, H2AFZ, HOXB7, IFT88, IL15RA, ISG20, ITGAE, KIAA0746, SERINC2, KIF13B, KLC1, LAMP3, LANCL1, LAP3, LEPREL1, LL22NC03-5H6.5, LOC100131861, SAMM50, LRRC41, LRRC47, MAP4, MAPKAPK5, MCM5, MCRS1, METRN, METTL3, MFHAS1, MMP3, MOSPD1, MRPL46, MTUS1, MYRIP, N4BP2L2, NAB1, NAT1, NDUFC1, NISCH, NUMB, OGT, OSBPL3, PAM, PBK, PDGFA, PEBP1, PGDS, PIGR, PIGT, PRDM2, PRELP, PSMA5, PSMD9, PSPC1, PTHLH, R3HCC1, RP3-377H14.5, RPLP0, RPLP0-like, RPS27L, RTN2, RYK, SAV1, SCAMP1, SERPINA1, SF3B1, SFPQ, SGCD, SLC25A3, SLC39A8, SMG7, SMURF2, SORD, SOX4, SPATA5L1, SQRDL, SRP72, SSNA1, STK3, SYNGR1, TAPBPL, TEGT, TES, TLN1, TMCC1, TMEM106C, TMEM16A, TMEM33, TMEM87A, TNFRSF10B, TNFSF10, TNIK, TRIM36, U2AF2, UBE2L6, UCP2, UNC84A, UQCRFS1, UQCRH, USP12, USP3, VPS41, WARS, WDR1, WDR68, XPO7, YBX1, ZC3H7B, ZMYM2, ZMYM5, ZNF117, and ZNF430 in the biological sample;
comparing the detected expression levels of the at least five genes from the biological sample with expression levels of the corresponding at least five genes when associated with a good disease prognosis expression profile and when associated with a bad disease prognosis expression profile; and
determining the prognosis of the subject having colon cancer based on said comparing.

2. The method according to claim 1, wherein said good disease prognosis expression profile comprises: (1) genes having expression levels below that of an average tumor sample expression level that are selected from the group consisting of AK023058*, AIP, ARL2BP, C1GALT1, CDC42BPA, C8orf70, CLN5, COBL, CYB5B, MOSPD1, DOCK9, EGFR, FKBP14, DND1, GREM2, GPR177, GALNS, GRB10, GRP, GSTA1, RP3-377H14.5, HOXB7, ZNF117, TNIK, LANCL1, METRN, LEPREL1, NAB1, NISCH, OGT, OSBPL3, PDGFA, PRDM2, PRELP, PSPC1, RECQL, RYK, SMURF2, TLN1, UNC84A, USP12, ZMYM2, ZMYM5, AL359599*, ARL4A, N4BP2L2, GLS, C19orf36, TMCC1, METTL3, TMEM16A, RTN2, SCAMP1, SF3B1, SOX4, STK3, ZNF430, C6orf15, C7orf10, CHST12, ETV1, ACSL4, FLJ10357, C5orf23, AA058828*, CDR2L, KLC1, MAP4, NUMB, PAM, PGDS, PTHLH, ZC3H7B, SAV1, SGCD, SYNGR1, TES, IFT88, TRIM36 and VPS41; and (2) genes having expression levels above that of an average tumor sample expression level that are selected from the group consisting of SERPINA1, RPLP0, RPLP0-like, CYB561, AKR1A1, AP3D1, ARL6IP4, OGFOD2, ASNA1, CFB, ERP29, SMG7, CASP1, CCNA2, LOC100131861, SAMM50, COX5A, CXCL11, DAZAP2, DDX23, FDFT1, COMMD4, GCHFR, GRHPR, GSR, ISG20, ITGAE, KIAA0746, SERINC2, FRYL, LRRC47, LAMP3, R3HCC1, MAPKAPK5, MCM5, MCRS1, TMEM106C, MMP3, MTUS1, LRRC41, NAT1, NDUFC1, YBX1, PEBP1, PIGR, PSMA5, SFPQ, SLC25A3, SLC39A8, SQRDL, SRP72, SSNA1, TAPBPL, TEGT, PBK, UCP2, UQCRH, XPO7, CCT2, CNOT7, DHX15, TMEM87A, ELP3, FAM82C, LL22NC03-5H6.5, DENND2D, WDR68, IL15RA, DENND2A, KIF13B, MFHAS1, SPATA5L1, MYRIP, PIGT, PSMD9, RPS27L, TEGT, TNFRSF10B, UBE2L6, USP3, ATP5B, CALML4, C1orf144, TMEM33, C12orf52, GHITM, H2AFZ, LAP3, MRPL46, SORD, CNPY2, TNFSF10, U2AF2, CMPK1, UQCRFS1, WARS and WDR1.

3. The method according to claim 1, wherein said bad disease prognosis expression profile comprises: (1) genes having expression levels below that of an average tumor sample expression level that are selected from the group consisting of SERPINA1, RPLP0, RPLP0-like, CYB561, AKR1A1, AP3D1, ARL6IP4, OGFOD2, ASNA1, CFB, ERP29, SMG7, CASP1, CCNA2, LOC100131861, SAMM50, COX5A, CXCL11, DAZAP2, DDX23, FDFT1, COMMD4, GCHFR, GRHPR, GSR, ISG20, ITGAE, KIAA0746, SERINC2, FRYL, LRRC47, LAMP3, R3HCC1, MAPKAPK5, MCM5, MCRS1, TMEM106C, MMP3, MTUS1, LRRC41, NAT1, NDUFC1, YBX1, PEBP1, PIGR, PSMA5, SERPINA1, SFPQ, SLC25A3, SLC39A8, SQRDL, SRP72, SSNA1, TAPBPL, TEGT, PBK, UCP2, UQCRH, XPO7, CCT2, CNOT7, DHX15, TMEM87A, ELP3, FAM82C, LL22NC03-5H6.5, DENND2D, WDR68, IL15RA, DENND2A, KIF13B, MFHAS1, SPATA5L1, MYRIP, PIGT, PSMD9, RPS27L, TNFRSF10B, UBE2L6, USP3, ATP5B, CALML4, C1orf144, TMEM33, C12orf52, GHITM, H2AFZ, LAP3, MRPL46, SORD, CNPY2, TNFSF10, U2AF2, CMPK1, UQCRFS1, WARS and WDR1 and (2) genes having expression levels above that of an average tumor sample expression level that are selected from the group consisting of AK023058*, AIP, ARL2BP, C1GALT1, CDC42BPA, C8orf70, CLN5, COBL, CYB5B, MOSPD1, DOCK9, EGFR, FKBP14, DND1, GREM2, GPR177, GALNS, GRB10, GRP, GSTA1, RP3-377H14.5, HOXB7, ZNF117, TNIK, LANCL1, METRN, LEPREL1, NAB1, NISCH, OGT, OSBPL3, PDGFA, PRDM2, PRELP, PSPC1, RECQL, RYK, SMURF2, TLN1, UNC84A, USP12, ZMYM2, ZMYM5, AL359599*, ARL4A, N4BP2L2, GLS, C19orf36, TMCC1, METTL3, TMEM16A, RTN2, SCAMP1, SF3B1, SOX4, STK3, ZNF430, C6orf15, C7orf10, CHST12, ETV1, ACSL4, FLJ10357, C5orf23, AA058828*, CDR2L, KLC1, MAP4, NUMB, PAM, PGDS, PTHLH, ZC3H7B, SAV1, SGCD, SYNGR1, TES, IFT88, TRIM36, and VPS41.

4. The method according to claim 1, wherein said determining comprises:
calculating a percentage of genes having an expression level associated with a good disease prognosis expression profile and a percentage of genes having an expression level associated with a bad disease prognosis expression profile in the sample, wherein a favorable prognosis for the subject exists when greater than 30% of the genes have expression levels associated with a good disease prognosis expression profile and less than 30% of the genes have expression levels associated with a bad disease prognosis expression profile, and wherein an unfavorable prognosis for the subject exists when greater than 30% of the genes have expression levels associated with a bad disease prognosis expression profile and less than 30% of the genes have expression levels associated with a good disease prognosis expression profile.

5. The method according to claim 1, wherein the at least five genes are selected from a group of 71 genes informative of colon cancer prognosis consisting of SLC25A3, DAZAP2, TEGT, ERP29, PSMA5, DDX23, LOC100131861, SAMM50, SFPQ, NISCH, CYB5B, TMEM106C, EGFR, MCRS1, SERPINA1, CCNA2, NDUFC1, COX5A, GCHFR, ITGAE, PRDM2, PDGFA, GSR, GRP, COMMD4, XPO7, YBX1, SRP72, UCP2, SLC39A8, NAB1, WDR68, CXCL11, RECQL, CASP1, PTHLH, UNC84A, MTUS1, KIAA0746, SERINC2, DOCK9, FRYL, MAPKAPK5, LRRC47, RQCD1, TNIK, RPLP0, RPLP0-like, CLN5, NAT1, CDC42BPA, GSTA1, ZMYM5, RYK, PIGT, CMPK1, SQRDL, FAM82C, CNOT7, LL22NC03-5H6.5, PSPC1, TAPBPL, METRN, PBK, MRPL46, FKBP14, C1GALT1, GREM2, GPR177, DND1, and PRELP.

6. The method according to claim 1, wherein the biological sample comprises colon cancer cells.

7. The method according to claim 6, wherein the colon cancer cells are from a stage I, II, III, or IV colon cancer tumor.

8. The method according to claim 1, wherein said detecting the expression level comprises:
measuring RNA expression level or protein expression level.

9. The method according to claim 8, wherein protein expression level is measured using a protein hybridization assay.

10. The method according to claim 8, wherein RNA expression level is measured using a nucleic acid hybridization assay or a nucleic acid amplification assay.

11. The method according to claim 10, wherein the nucleic acid hybridization assay is carried out using an array comprising a plurality of nucleic acid probes.

12. The method according to claim 11, wherein said array comprises a plurality of nucleic acid probes, each nucleic acid probe comprising a nucleotide sequence that is complementary to at least a portion of a nucleotide sequence of a gene selected from the group of 176 genes informative of colon cancer outcome.

13. The method according to claim 11, wherein said array comprises a plurality of nucleic acid probes, each nucleic acid probe comprising a nucleotide sequence that is complementary to at least a portion of a nucleotide sequence of a gene selected from the group of 71 genes informative of colon cancer outcome.

14. The method according to claim 10, wherein the nucleic acid amplification assay is a semi-quantitative or quantitative real-time polymerase chain reaction (RT-PCR) assay.

15. The method according to claim 1 further comprising:
performing one or more additional analyses, wherein said additional analyses are selected from the group consisting of detecting microsatellite instability, measuring DNA promoter methylation level, screening one or more mutations in one or more colon cancer oncogenes or tumor suppressor genes in the sample, and combinations thereof and
determining the prognosis of the subject having colon cancer based on said comparing the detected gene expression level and said performing one or more additional analyses.

16. The method according to claim 15, wherein said performing comprises:
detecting microsatellite instability using an NCI 5-marker panel, wherein a favorable prognosis exists when a microsatellite instability-low status is detected.

17. The method according to claim 15, wherein said performing comprises:
measuring methylation level of the lecithin:retinol acyl transferase gene promoter nucleotide sequence, or region upstream thereof, wherein a favorable prognosis exists when an increase in the methylation level of the lecithin:retinol acyl transferase gene promoter nucleotide sequence, or region upstream thereof, is measured.

18. The method according to claim 15, wherein said performing comprises:
screening mutational status of one or more colon cancer oncogenes or tumor-suppressor genes selected from the group consisting of K-ras, B-raf, APC, p53, and PIK3CA, wherein an unfavorable prognosis exists when mutations in one or more of the colon cancer oncogenes or tumor suppressor genes are identified.

19. The method according to claim 1 further comprising:
preparing a personalized genomic profile for a colon cancer patient based on said determining.

20. The method according to claim 19 further comprising:
establishing a treatment plan for the colon cancer patient based on said personalized genomic profile.

21. The method according to claim 20, wherein the treatment plan comprises surgery, individual therapy, chemotherapy, or a combination thereof.

22. The method according to claim 20 further comprising:
treating said colon cancer patient based on the treatment plan.

23. A method for determining the prognosis of a subject having colon cancer, said method comprising:
obtaining a biological sample from the subject;
detecting the expression level of at least five genes selected from a group of 101 genes informative of colon cancer prognosis consisting of NARS, WDR1, WARS, CCT4, ATP5B, SORD, UBE2L6, PSME2, AIP, RRM2, LRRC41, CCT2, TAF9, HDAC5, SVIL, CCNB2, DBN1, PBX2, RFC5, IDE, MAD2L1, PSMA4, NDUFC1, IVD, PPIH, NEO1, CXCL10, FXN, GABBR1, ARHGAP8, LOC553158, HOXA4, COMMD4, DFFB, KLF12, GLMN, CASP7, PIR, ATP5G3, ACTN1, DDOST, TAPBP, RGL2, CYB561, TUSC3, C3orf63, GRB10, NR2F1, WDR68, CXCL2, CNPY2, CASP1, INDO, PFKM, CXCL11, MCAM, MAP2K5, MRPS11, NOLC1, CD59, CAMSAP1L1, SHANK2, KLC1, EMP1, C1orf95, GMDS, RPLP0, RPLP0-like, PDLIM4, PAM, TM4SF1, BEX4, ADORA1, FAM48A, ITM2B, PREB, CMPK1, LAP3, FAM82C, AACS, RP5-1077B9.4, NUP37, RHBDF1, PBK, TIPIN, TMEM204, ALG6, NPR3, SCD5, FLJ13236, GPATCH4, GREM2, RPL22, KLHL3, C15orf44, USP3, TNS1, ZBTB20, RTN2, FLJ10357, and CALML4, in the biological sample;
comparing the detected expression level of the at least five genes from the biological sample with expression levels of the corresponding at least five genes when associated with a good disease prognosis expression profile and when associated with a bad disease prognosis expression profile; and
determining the prognosis of the subject having colon cancer based on said comparing.

24. The method according to claim 23, wherein said good disease prognosis expression profile comprises: (1) genes having expression levels below that of an average tumor sample expression level that are selected from the group consisting of ACTN1, ADORA1, ARHGAP8, LOC553158, BEX4, C1orf95, C3orf63, CAMSAP1L1, CD59, CNPY2, DBN1, FAM48A, FLJ10357, GPATCH4, GRB10, GREM2, HDAC5, HOXA4, ITM2B, KLC1, KLF12, KLHL3, NPR3, PAM, PBX2, PDLIM4, PIR, RGL2, RHBDF1, RP5-1077B9.4, RTN2, SCD5, SHANK2, SVIL, TAPBP, TIPIN, TM4SF1, TMEM204, TNS1, TUSC3, and ZBTB20 and (2) genes having expression levels above that of an average tumor sample expression level that are selected from the group consisting of NARS, WDR1, WARS, CCT4, ATP5B, SORD, UBE2L6, PSME2, AIP, RRM2, LRRC41, CCT2, TAF9, CCNB2, RFC5, IDE, MAD2L1, PSMA4, NDUFC1, IVD, PPIH, NEO1, CXCL10, FXN, GABBR1, COMMD4, DFFB, GLMN, CASP7, ATP5G3, DDOST, CYB561, NR2F1, WDR68, CXCL2, CASP1, INDO, PFKM, CXCL11, MCAM, MAP2K5, MRPS11, NOLC1, EMP1, GMDS, RPLP0, RPLP0-like, PREB, CMPK1, LAP3, FAM82C, AACS, NUP37, PBK, ALG6, FLJ13236, RPL22, C15orf44, USP3, and CALML4.

25. The method according to claim 23, wherein said bad disease prognosis expression profile comprises: (1) genes having expression levels below that of an average tumor sample expression level that are selected from the group consisting of NARS, WDR1, WARS, CCT4, ATP5B, SORD, UBE2L6, PSME2, AIP, RRM2, LRRC41, CCT2, TAF9, CCNB2, RFC5, IDE, MAD2L1, PSMA4, NDUFC1, IVD, PPIH, NEO1, CXCL10, FXN, GABBR1, COMMD4, DFFB, GLMN, CASP7, ATP5G3, DDOST, CYB561, NR2F1, WDR68, CXCL2, CASP1, INDO, PFKM, CXCL11, MCAM, MAP2K5, MRPS11, NOLC1, EMP1, GMDS, RPLP0, RPLP0-like, PREB, CMPK1, LAP3, FAM82C, AACS, NUP37, PBK, ALG6, FLJ13236, RPL22, C15orf44, USP3, and CALML4 and (2) genes having expression levels above that of an average tumor sample expression level that are selected from the group consisting of ACTN1, ADORA1, ARHGAP8, LOC553158, BEX4, C1orf95, C3orf63, CAMSAP1L1, CD59, CNPY2, DBN1, FAM48A, FLJ10357, GPATCH4, GRB10, GREM2, HDAC5, HOXA4, ITM2B, KLC1, KLF12, KLHL3, NPR3, PAM, PBX2, PDLIM4, PIR, RGL2, RHBDF1, RP5-1077B9.4, RTN2, SCD5, SHANK2, SV1L, TAPBP, TIPIN, TM4SF1, TMEM204, TNS1, TUSC3, and ZBTB20.

26. The method according to claim 23, wherein said determining comprises:
calculating a percentage of genes having an expression level associated with a good disease prognosis expression profile and a percentage of genes having an expression level associated with a bad disease prognosis expression profile in the sample, wherein a favorable prognosis for the subject is determined when greater than 30% of the genes have expression levels associated with a good disease prognosis expression profile and less than 30% of the genes have expression levels associated with a bad disease prognosis expression profile, and wherein an unfavorable prognosis for the subject is determined when greater than 30% of the genes have expression levels associated with a bad disease prognosis expression profile and less than 30% of the genes have expression levels associated with a good disease prognosis expression profile.

27. The method according to claim 23, wherein the biological sample comprises colon cancer cells.

28. The method according to claim 27, wherein the colon cancer cells are collected from a stage I, II, III, or IV colon cancer tumor.

29. The method according to claim 23, wherein said detecting the expression level comprises:
measuring RNA expression level or protein expression level.

30. The method according to claim 29, wherein protein expression level is measured using a protein hybridization assay.

31. The method according to claim 29, wherein RNA expression level is measured using a nucleic acid hybridization assay or a nucleic acid amplification assay.

32. The method according to claim 31, wherein the nucleic acid hybridization assay is carried out using an array comprising a plurality of nucleic acid probes.

33. The method according to claim 32, wherein said array comprises a plurality of nucleic acid probes, each nucleic acid probe comprising a nucleotide sequence that is complementary to at least a portion of a nucleotide sequence of a gene selected from the group of 101 genes informative of colon cancer outcome.

34. The method according to claim 31, wherein the nucleic acid amplification assay is a semi-quantitative or quantitative real-time polymerase chain reaction (RT-PCR) assay.

35. The method according to claim 23 further comprising:
preparing a personalized genomic profile for a colon cancer patient based on said determining.

36. The method according to claim 35 further comprising:
establishing a treatment plan for the colon cancer patient based on said personalized genomic profile.

37. The method according to claim 36, wherein the treatment plan comprises surgery, individual therapy, chemotherapy, or a combination thereof.

38. The method according to claim 36 further comprising:
treating said colon cancer patient based on the treatment plan.

39. A method of identifying an agent that improves the prognosis of a subject having colon cancer, said method comprising:
administering an agent to the subject having colon cancer;
obtaining a first biological sample from the subject before said administering and a second biological sample from the subject after said administering;
detecting the expression level of at least five genes selected from a group of 176 genes informative of colon cancer prognosis consisting of SLC25A3, WDR1, WARS, DAZAP2, TEGT, H2AFZ, SF3B1, ERP29, PSMA5, ATP5B, DHX15, SOX4, DDX23, SORD, LOC100131861, SAMM50, SFPQ, NISCH, CYB5B, UBE2L6, MCM5, TMEM106C, AIP, SMG7, AKR1A1, LRRC41, CCT2, EGFR, LANCL1, ASNA1, ARL2BP, UQCRH, N4BP2L2, CFB, ACSL4, MCRS1, TNFSF10, TES, ZMYM2, SERPINA1, KIF13B, TLN1, CCNA2, NDUFC1, COX5A, STK3, PIGR, SYNGR1, IFT88, HOXB7, GCHFR, ARL4A, ITGAE, PRDM2, C8orf70, PEBP1, PDGFA, LAMP3, SMURF2, GSR, MMP3, ZC3H7B, GRP, GALNS, COMMD4, PGDS, ZNF430, IL15RA, OGT, ZNF117, PSMD9, XPO7, YBX1, SRP72, UQCRFS1, UCP2, NUMB, GHITM, SLC39A8, NAB1, TNFRSF10B, GRB10, WDR68, OSBPL3, CNPY2, CXCL11, SSNA1, RECQL, VPS41, FDFT1, AP3D1, CASP1, PTHLH, C1orf144, UNC84A, MTUS1, TMEM87A, KIAA0746, SERINC2, SCAMP1, DOCK9, FRYL, R3HCC1, MAPKAPK5, LRRC47, PAM, COBL, TNIK, CDR2L, USP12, TMCC1, MFHAS1, METTL3, KLC1, MYRIP, RPLP0, RPLP0-like, CLN5, C19orf36, NAT1, CDC42BPA, SGCD, GSTA1, AL359599*, ZMYM5, GRHPR, RYK, CYB561, PIGT, CMPK1, LAP3, SQRDL, RPS27L, FAM82C, CNOT7, LL22NC03-5H6.5, SAV1, PSPC1, U2AF2, TMEM33, LEPREL1, TAPBPL, TMEM16A, MOSPD1, CHST12, METRN, C5orf23, PBK, MRPL46, FKBP14, C1GALT1, C7orf10, TRIM36, ARL6IP4, OGFOD2, GREM2, DENND2D, ELP3, C6orf15, GLS, USP3, C12orf52, ETV1, GPR177, AA058828*, DND1, AK023058*, SPATA5L1, RP3-377H14.5, MAP4, ISG20, RTN2, PRELP, DENND2A, FLJ10357, and CALML4;
determining increases or decreases in the expression levels of the at least five genes in the second sample compared to the first sample; and
identifying an agent that improves the prognosis of a subject having colon cancer based on said determining.

40. The method according to claim 39, wherein an agent that increases the expression level of any one or more genes selected from the group consisting of SERPINA1, RPLP0, RPLP0-like, CYB561, AKR1A1, AP3D1, ARL6IP4, OGFOD2, ASNA1, CFB, ERP29, SMG7, CASP1, CCNA2, LOC100131861, SAMM50, COX5A, CXCL11, DAZAP2, DDX23, FDFT1, COMMD4, GCHFR, GRHPR, GSR, ISG20, ITGAE, KIAA0746, SERINC2, FRYL, LRRC47, LAMP3, R3HCC1, MAPKAPK5, MCM5, MCRS1, TMEM106C, MMP3, MTUS1, LRRC41, NAT1, NDUFC1, YBX1, PEBP1, PIGR, PSMA5, SFPQ, SLC25A3, SLC39A8, SQRDL, SRP72, SSNA1, TAPBPL, TEGT, PBK, UCP2, UQCRH, XPO7, CCT2, CNOT7, DHX15, TMEM87A, ELP3, FAM82C, LL22NC03-5H6.5, DENND2D, WDR68, IL15RA, DENND2A, KIF13B, MFHAS1, SPATA5L1, MYRIP, PIGT, PSMD9, RPS27L, TNFRSF10B, UBE2L6, USP3, ATP5B, CALML4, C1orf144, TMEM33, C12orf52, GHITM, H2AFZ, LAP3, MRPL46, SORD, CNPY2, TNFSF10, U2AF2, CMPK1, UQCRFS1, WARS, and WDR1, and/or decreases the expression level of any one or more genes selected from the group consisting of AK023058*, AIP, ARL2BP, C1GALT1, CDC42BPA, C8orf70, CLN5, COBL, CYB5B, MOSPD1, DOCK9, EGFR, FKBP14, DND1, DND1, GREM2, GPR177, GALNS, GRB10, GRP, GSTA1, RP3-377H14.5, HOXB7, ZNF117, TNIK, LANCL1, METRN, LEPREL1, NAB1, NISCH, OGT, OSBPL3, PDGFA, PRDM2, PRELP, PSPC1, RECQL, RYK, SMURF2, TLN1, UNC84A, USP12, ZMYM2, ZMYM5, AL359599*, ARL4A, N4BP2L2, GLS, C19orf36, TMCC1, METTL3, TMEM16A, RTN2, SCAMP1, SF3B1, SOX4, STK3, ZNF430, C6orf15, C7orf10, CHST12, ETV1, ACSL4, FLJ10357, C5orf23, AA058828*, CDR2L, KLC1, MAP4, NUMB, PAM, PGDS, PTHLH, ZC3H7B, SAV1, SGCD, SYNGR1, TES, IFT88, TRIM36, and VPS41 is identified as an agent that improves the prognosis of a subject having colon cancer.

41. The method according to claim 39, wherein the at least five genes are selected from a group of 71 genes informative of colon cancer prognosis consisting of SLC25A3, DAZAP2, TEGT, ERP29, PSMA5, DDX23, LOC100131861, SAMM50, SFPQ, NISCH, CYB5B, TMEM106C, EGFR, MCRS1, SERPINA1, CCNA2, NDUFC1, COX5A, GCHFR, ITGAE, PRDM2, PDGFA, GSR, GRP, COMMD4, XPO7, YBX1, SRP72, UCP2, SLC39A8, NAB1, WDR68, CXCL11, RECQL, CASP1, PTHLH, UNC84A, MTUS1, KIAA0746, SERINC2, DOCK9, FRYL, MAPKAPK5, LRRC47, RQCD1, TNIK, RPLP0, RPLP0-like, CLN5, NAT1, CDC42BPA, GSTA1, ZMYM5, RYK, PIGT, CMPK1, SQRDL, FAM82C, CNOT7, LL22NC03-5H6.5, PSPC1, TAPBPL, METRN, PBK, MRPL46, FKBP14, C1GALT1, GREM2, GPR177, DND1, and PRELP.

42. The method according to claim 39, wherein the biological sample comprises colon cancer cells.

43. The method according to claim 42, wherein the colon cancer cells are from a stage I, II, III, or IV colon cancer tumor.

44. The method according to claim 39, wherein said detecting the expression level comprises:
measuring RNA expression level or protein expression level.

45. The method according to claim 44, wherein protein expression level is detected using a protein hybridization assay.

46. The method according to claim 44, wherein RNA expression level is detected using a nucleic acid hybridization assay or a nucleic acid amplification assay.

47. The method according to claim 46, wherein the nucleic acid hybridization assay is carried out using an array comprising a plurality of nucleic acid probes.

48. The method according to claim 47, wherein said array comprises a plurality of nucleic acid probes, each nucleic acid probe comprising a nucleotide sequence that is complementary to at least a portion of a nucleotide sequence of a gene selected from the group of 176 genes informative of colon cancer outcome consisting of SLC25A3, WDR1, WARS, DAZAP2, TEGT, H2AFZ, SF3B1, ERP29, PSMA5, ATP5B, DHX15, SOX4, DDX23, SORD, LOC100131861, SAMM50, SFPQ, NISCH, CYB5B, UBE2L6, MCM5, TMEM106C, AIP, SMG7, AKR1A1, LRRC41, CCT2, EGFR, LANCL1, ASNA1, ARL2BP, UQCRH, N4BP2L2, CFB, ACSL4, MCRS1, TNFSF10, TES, ZMYM2, SERPINA1, KIF13B, TLN1, CCNA2, NDUFC1, COX5A, STK3, PIGR, SYNGR1, IFT88, HOXB7, GCHFR, ARL4A, ITGAE, PRDM2, C8orf70, PDGFA, LAMP3, SMURF2, GSR, MMP3, ZC3H7B, GRP, GALNS, COMMD4, PGDS, ZNF430, IL15RA, OGT, ZNF117, PSMD9, XPO7, YBX1, SRP72, UQCRFS1, UCP2, NUMB, GHITM, SLC39A8, NAB1, TNFRSF10B, GRB10, WDR68, OSBPL3, CNPY2, CXCL11, SSNA1, RECQL, VPS41, FDFT1, AP3D1, CASP1, PTHLH, PEBP1, C1orf144, UNC84A, MTUS1, TMEM87A, KIAA0746, SERINC2, SCAMP1, DOCK9, FRYL, R3HCC1, MAPKAPK5, LRRC47, PAM, COBL, TNIK, CDR2L, USP12, TMCC1, MFHAS1, METTL3, KLC1, MYRIP, RPLP0, RPLP0-like, CLN5, C19orf36, NAT1, CDC42BPA, SGCD, GSTA1, AL359599*, ZMYM5, GRHPR, RYK, CYB561, PIGT, CMPK1, LAP3, SQRDL, RPS27L, FAM82C, CNOT7, LL22NC03-5H6.5, SAV1, PSPC1, U2AF2, TMEM33, LEPREL1, TAPBPL, TMEM16A, MOSPD1, CHST12, METRN, C5orf23, PBK, MRPL46, FKBP14, C1GALT1, C7orf10, TRIM36, ARL6IP4, OGFOD2, GREM2, DENND2D, ELP3, C6orf15, GLS, USP3, C12orf52, ETV1, GPR177, AA058828*, DND1, AK023058*, SPATA5L1, RP3-377H14.5, MAP4, ISG20, RTN2, PRELP, DENND2A, FLJ10357, and CALML4.

49. The method according to claim 47, wherein said array comprises a plurality of nucleic acid probes, each nucleic acid probe comprising a nucleotide sequence that is complementary to at least a portion of a nucleotide sequence of a gene selected from a group of 71 genes informative of colon cancer outcome consisting of SLC25A3, DAZAP2, TEGT, ERP29, PSMA5, DDX23, LOC100131861, SAMM50, SFPQ, NISCH, CYB5B, TMEM106C, EGFR, MCRS1, SERPINA1, CCNA2, NDUFC1, COX5A, GCHFR, ITGAE, PRDM2, PDGFA, GSR, GRP, COMMD4, XPO7, YBX1, SRP72, UCP2, SLC39A8, NAB1, WDR68, CXCL11, RECQL, CASP1, PTHLH, UNC84A, MTUS1, KIAA0746, SERINC2, DOCK9, FRYL, MAPKAPK5, LRRC47, RQCD1, TNIK, RPLP0, RPLP0-like, CLN5, NAT1, CDC42BPA, GSTA1, ZMYM5, RYK, PIGT, CMPK1, SQRDL, FAM82C, CNOT7, LL22NC03-5H6.5, PSPC1, TAPBPL, METRN, PBK, MRPL46, FKBP14, C1GALT1, GREM2, GPR177, DND1, and PRELP.

50. The method according to claim 46, wherein the nucleic acid amplification assay is a semi-quantitative or quantitative real-time polymerase chain reaction (RT-PCR) assay.

51. A collection of 71 genes having expression levels informative for predicting a prognosis of a patient having colon cancer, said collection of genes comprising: SLC25A3, DAZAP2, TEGT, ERP29, PSMA5, DDX23, LOC100131861, SAMM50, SFPQ, NISCH, CYB5B, TMEM106C, EGFR, MCRS1, SERPINA1, CCNA2, NDUFC1, COX5A, GCHFR, ITGAE, PRDM2, PDGFA, GSR, GRP, COMMD4, XPO7, YBX1, SRP72, UCP2, SLC39A8, NAB1, WDR68, CXCL11, RECQL, CASP1, PTHLH, UNC84A, MTUS1, KIAA0746, SERINC2, DOCK9, FRYL, MAPKAPK5, LRRC47, RQCD1, TNIK, RPLP0, RPLP0-like, CLN5, NAT1, CDC42BPA, GSTA1, ZMYM5, RYK, PIGT, CMPK1, SQRDL, FAM82C, CNOT7, LL22NC03-5H6.5, PSPC1, TAPBPL, METRN, PBK, MRPL46, FKBP14, C1GALT1, GREM2, GPR177, DND1, and PRELP

52. The collection of genes according to claim 51, wherein said collection further comprises AA058828*, ACSL4, AIP, AK023058*, AKR1A1, AL359599*, AP3D1, ARL2BP, ARL4A, ARL6IP4, OGFOD2, ASNA1, ATP5B, C12orf52, C19orf36, C1orf144, C5orf23, C6orf15, C7orf10, C8orf70, CALML4, CCT2, CDR2L, CFB, CHST12, CNPY2, COBL, CYB561, DENND2A, DENND2D, DHX15, DND1, ELP3, ETV1, FDFT1, FLJ10357, GALNS, GHITM, GLS, GRB10, GRHPR, H2AFZ, HOXB7, IFT88, IL15RA, ISG20, KIAA0746, SERINC2, KIF13B, KLC1, LAMP3, LANCL1, LAP3, LEPREL1, LRRC41, MAP4, MCM5, METTL3, MFHAS1, MMP3, MOSPD1, MYRIP, N4BP2L2, NUMB, OGT, OOSBPL3, PAM, PEBP1, PGDS, PIGR, PSMD9, R3HCC1, RP3-377H14.5, RPS27L, RTN2, SAV1, SCAMP1, SF3B1, SGCD, SLC39A8, SMG7, SMURF2, SORD, SOX4, SPATA5L1, SSNA1, STK3, SYNGR1, TEGT, TES, TLN1, TMCC1, TMEM16A, TMEM33, TMEM87A, TNFRSF10B, TNFSF10, TRIM36, U2AF2, UBE2L6, UCP2, UQCRFS1, UQCRH, USP12, USP3, VPS41, WARS, WDR1, ZC3H7B, ZMYM2, ZNF117, and ZNF430.

53. An array comprising a plurality of nucleic acid probes, each nucleic acid probe comprising a nucleotide sequence that is complementary to at least a portion of a nucleotide sequence of a gene selected from the collection of genes of claim 51.

54. A collection of 101 genes having expression levels informative for predicting a prognosis of a patient having colon cancer, said collection of genes comprising: AACS, ACTN1, ADORA1, AIP, ALG6, ARHGAP8, LOC553158, ATP5B, ATP5G3, BEX4, C15orf44, C1orf95, C3orf63, CALML4, CAMSAP1L1, CASP1, CASP7, CCNB2, CCT2, CCT4, CD59, CMPK1, CNPY2, COMMD4, CXCL10, CXCL11, CXCL2, CYB561, DBN1, DDOST, DFFB, EMP1, FAM48A, FAM82C, FLJ10357, FLJ13236, FXN, GABBR1, GLMN, GMDS, GPATCH4, GRB10, GREM2, HDAC5, HOXA4, IDE, INDO, ITM2B, IVD, KLC1, KLF12, KLHL3, LAP3, LRRC41, MAD2L1, MAP2K5, MCAM, MRPS11, NARS, NDUFC1, NEO1, NOLC1, NPR3, NR2F1, NUP37, PAM, PBK, PBX2, PDLIM4, PFKM, PIR, PPIH, PREB, PSMA4, PSME2, RFC5, RGL2, RHBDF1, RP5-1077B9.4, RPL22, RPLP0, RPLP0-like, RRM2, RTN2, SCD5, SHANK2, SORD, SVIL, TAF9, TAPBP, TIPIN, TM4SF1, TMEM204, TNS1, TUSC3, UBE2L6, USP3, WARS, WDR1, WDR68, and ZBTB20.

55. An array comprising a plurality of nucleic acid probes, each nucleic acid probe comprising a nucleotide sequence that is complementary to at least a portion of a nucleotide sequence of a gene selected from the group consisting of the collection of genes of claim 54.

56. A method for determining a subject's predisposition to having colon cancer, said method comprising:
obtaining a biological sample from the subject;
detecting the expression level of at least five genes selected from a collection of 176 genes informative of colon cancer predisposition consisting of ACSL4, RQCD1, AA058828*, AIP, AKR1A1, AP3D1, ARL2BP, ARL4A, ARL6IP4, OGFOD2, ASNA1, ATP5B, C12orf52, C19orf36, C1GALT1, C1orf144, C5orf23, C6orf15, C7orf10, C8orf70, CALML4, CASP1, CCNA2, CCT2, CDC42BPA, AK023058*, CDR2L, CFB, CHST12, CLN5, CMPK1, CNOT7, CNPY2, COBL, COMMD4, COX5A, CXCL11, CYB561, CYB5B, DAZAP2, DDX23, DENND2A, DENND2D, DHX15, AL359599*, DND1, DOCK9, EGFR, ELP3, ERP29, ETV1, FAM82C, FDFT1, FKBP14, FLJ10357, FRYL, GALNS, GCHFR, GHITM, GLS, GPR177, GRB10, GREM2, GRHPR, GRP, GSR, GSTA1, H2AFZ, HOXB7, IFT88, IL15RA, ISG20, ITGAE, KIAA0746, SERINC2, KIF13B, KLC1, LAMP3, LANCL1, LAP3, LEPREL1, LL22NC03-5H6.5, LOC100131861, SAMM50, LRRC41, LRRC47, MAP4, MAPKAPK5, MCM5, MCRS1, METRN, METTL3, MFHAS1, MMP3, MOSPD1, MRPL46, MTUS1, MYRIP, N4BP2L2, NAB1, NAT1, NDUFC1, NISCH, NUMB, OGT, OSBPL3, PAM, PBK, PDGFA, PEBP1, PGDS, PIGR, PIGT, PRDM2, PULP, PSMA5, PSMD9, PSPC1, PTHLH, R3HCC1, RP3-377H14.5, RPLP0, RPLP0-like, RPS27L, RTN2, RYK, SAV1, SCAMP1, SERPINA1, SF3B1, SFPQ, SGCD, SLC25A3, SLC39A8, SMG7, SMURF2, SORD, SOX4, SPATA5L1, SQRDL, SRP72, SSNA1, STK3, SYNGR1, TAPBPL, TEGT, TES, TLN1, TMCC1, TMEM106C, TMEM16A, TMEM33, TMEM87A, TNFRSF10B, TNFSF10, TNIK, TRIM36, U2AF2, UBE2L6, UCP2, UNC84A, UQCRFS1, UQCRH, USP12, USP3, VPS41, WARS, WDR1, WDR68, XPO7, YBX1, ZC3H7B, ZMYM2, ZMYM5, ZNF117, and ZNF430;
comparing the detected expression level of the at least five genes from said sample with the expression levels of the corresponding at least five genes when associated with a having a predisposition to colon cancer; and
determining the subject's predisposition to having colon cancer based on said comparing.
